(19) United States
(12) Patent Application Publication
(10) Pub. No.: US 2026/0207565 A1
(43) Pub. Date: Jul. 23, 2026

(54) COMPOUNDS THAT INHIBIT PKMYT1

(71) Applicant: Exelixis, Inc., Alameda, CA (US)

(72) Inventors: Wei Xu, Alameda, CA (US); Paige Mahaney, Alameda, CA (US); Benjamin Boswell, Alameda, CA (US); David Cole, Alameda, CA (US); Paul Gillespie, Alameda, CA (US); Faming Jiang, Alameda, CA (US); Xiaodong Lin, Alameda, CA (US); Sunghoon Ma, Alameda, CA (US); Jack Maung, Alameda, CA (US); Lynne Canne Bannen, Alameda, CA (US); Danny Ng, Alameda, CA (US); Jian Payandeh, Alameda, CA (US); Derek Piper, Alameda, CA (US); Sandeep Raikar, Alameda, CA (US); Alice Rico, Alameda, CA (US); Justin Salvant, Alameda, CA (US); Andre St. Amant, Alameda, CA (US); Yong Wang, Alameda, CA (US); Zhensheng Zhao, Alameda, CA (US); Guosheng Wu, Louisville, KY (US); Ryan L. Gonciarz, Alameda, CA (US)

(21) Appl. No.: 19/140,464

(22) PCT Filed: Dec. 19, 2023
(86) PCT No.: PCT/US2023/084893

§ 371 Date: Jun. 18, 2025

Related U.S. Application Data
(60) Provisional application No. 63/476,234, filed on Dec. 20, 2022.
(60) Provisional application No. 63/581,132, filed on Sep. 7, 2023.

(51) Int. Cl. A61K 31/437; A61K 31/404; A61K 31/416; A61K 31/4184; A61K 31/4192; A61K 31/444; C07D 209/42; C07D 231/56; C07D 235/08; C07D 249/18; C07D 471/04

(52) U.S. Cl. CPC A61K 31/437; A61K 31/404; A61K 31/416; A61K 31/4184; A61K 31/4192; A61K 31/444; C07D 209/42; C07D 231/56; C07D 235/08; C07D 249/18; C07D 471/04

(57) ABSTRACT

Disclosed herein are compounds according to Formula (I). Compounds according to Formula I inhibit PKMYT1 and are useful for the treatment of cancer and other disease. The present invention also provides methods for making compounds as mentioned above, and compositions which contain these compounds.

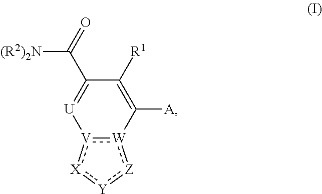

### CROSS-REFERENCES TO RELATED APPLICATIONS

The present application claims priority to U.S. Provisional Pat. Appl. No. 63/476,234, filed on Dec. 20, 2022, and U.S. Provisional Pat. Appl. No. 63/581,132, filed on Sep. 7, 2023, which applications are incorporated herein by reference in their entirety.

### FIELD OF THE INVENTION

The present disclosure relates to compounds that inhibit protein kinase, membrane associated tyrosine/threonine 1 (PKMYT1). The disclosure also provides processes for preparing these compounds, pharmaceutical compositions comprising these compounds, and methods of using these compounds to treat diseases, disorders, or conditions responsive to inhibition of PKMYT1.

### BACKGROUND OF THE INVENTION

Protein kinase, membrane associated tyrosine/threonine 1 (PKMYT1) is a member of the WEE family of serine/threonine kinases. PKMYT1 phosphorylates threonine 14 (Thr14) of cyclin-dependent kinase 1 (CDK1), inhibiting its ability to trigger mitosis. Genetically vulnerable tumors that lack a functional PKMYT1 lose major checkpoint regulation, resulting in cell death due to hyperactive CDK1, unscheduled mitosis and catastrophic DNA damage. Abnormal expression of PKMYT1 is associated with certain types of cancers including CCNE1-amplified cancers.

The CCNE1 locus encodes the protein cyclin E1, which complexes with cyclin-dependent kinase 2 (CDK2) and drives cells from the G1 phase to the S phase. Amplification of the CCNE1 locus on chromosome 19q12 is prevalent in multiple tumor types. Disrupting checkpoint regulation via PKMYT1 inhibition may therefore be particularly advantageous in promoting the death of target cancer cells exhibiting irregular expression of CCNE1 and/or mutations in cyclin regulators such as F-box/WD repeat-containing protein 7 (FBXW7) and protein phosphatase 2 regulatory subunit A alpha (PPP2R1A). To date, small molecule PKMYT1 inhibitors have been lacking. Thus, a need exists for compounds that inhibit PKMYT1 for the treatment of cancers.

### SUMMARY OF THE INVENTION

In one aspect, the present disclosure provides novel compounds that are PKMYT1 inhibitors.

In one aspect, the present disclosure provides a compound according to Formula I:

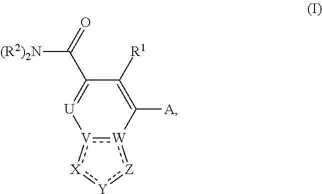

or a pharmaceutically acceptable salt thereof,
wherein:
each [symbol] represents a single bond, a double bond, or a delocalized π bond;
U is selected from the group consisting of N and CH;
V and W are independently selected from the group consisting of N and C;
X is selected from the group consisting of CR³, N. NR^{3a}, S, and O;
Y is selected from the group consisting of CR⁴, N. NR^{4a}, S, and O;
Z is selected from the group consisting of CR⁵, N, NR^{5a}, S, and O;
ring A is
3- to 14-membered carbocyclyl substituted with 1, 2, 3, 4, or 5 R⁶, or
5- to 14-membered heterocyclyl comprising at least one oxygen atom, nitrogen atom, or sulfur atom, wherein the heterocyclyl is substituted with 0, 1, 2, 3, or 4 R⁶;
R¹ is selected from the group consisting of amino, hydroxy, and hydrogen; each R² is independently selected from the group consisting of hydrogen and C₁₋₆ alkyl;
R³, R⁴, and R⁵ are independently selected from the group consisting of hydrogen, amino, hydroxy, halogen, cyano, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, —NO₂, —CO(C₁₋₆ alkyl), —COOH, —COO(C₁₋₆ alkyl), CON(R⁷)₂, -L-(5- to 12-membered heterocyclyl), -L-(5- to 14-membered heteroaryl, and -L-(C₆₋₁₄ aryl), wherein the 5- to 12-membered heterocyclyl, 5- to 14-membered heteroaryl, and C₆₋₁₄ aryl are independently substituted with 0, 1, 2, 3, or 4 R⁶;
each L is independently selected from the group consisting of a covalent bond, —O—, C₁₋₆ alkylene, and NR^{a};
any two adjacent R³, R⁴, and R⁵ are optionally taken together with the carbon atoms to which they are attached to form a 5- or 6-membered carbocyle or heterocycle, each of which is optionally substituted with 0, 1, 2, 3, or 4 R⁶.
R^{3a}, R^{4a}, R^{5a}, and R⁷ are independently selected from the group consisting of hydrogen, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, -L-(5- to 12-membered heterocyclyl), -L-(5- to 14-membered heteroaryl, and -L-(C₆₋₁₄ aryl), wherein the C₃₋₈ cycloalkyl, 5- to 12-membered heterocyclyl, 5- to 14-membered heteroaryl, and C₆₋₁₄ aryl are independently substituted with 0, 1, 2, 3, or 4 R⁶;
each R⁶ is independently selected from the group consisting of hydroxy, amino, cyano, halogen, C₁₋₆ alkyl, C₁₋₆ haloalkyl, C₁₋₆ hydroxyalkyl, C₁₋₆ alkoxy, oxo, —NO₂, —CHO, —CO(C₁₋₆ alkyl). —COOR^{a}, —CON(R^{a})₂, —SO₂(C₁₋₆ alkyl), and —SO₂N(R^{a})₂; and each R^{a} is independently selected from the group consisting of hydrogen, C₁₋₆ alkyl, and C₁₋₆ haloalkyl.

In some embodiments, ring A is substituted with at least one R⁶ that is selected from the group consisting of hydroxy, amino, cyano, C₁₋₆ alkyl, C₁₋₆ haloalkyl. C₁₋₆ hydroxyalkyl, oxo, —NO₂, —CHO, —CO(C₁₋₆ alkyl), —COOR^{a}, —CON(R^{a})₂, and —SO₂N(R^{a})₂ when ring A is a carbocycle and R¹ and each R² are hydrogen.

In some embodiments, ring A is substituted with at least one R⁶ that is selected from the group consisting of hydroxy, amino, cyano, halogen, C₁₋₆ haloalkyl. C₁₋₆ hydroxyalkyl, oxo, —NO₂, —CHO, —CO(C₁₋₆ alkyl), —COOR^{a}, —CON(R^{a})₂, —SO₂(C₁₋₆ alkyl), and —SO₂N(R^{a})₂ when ring A is a heterocycle and R¹ and each R² are hydrogen.

Another aspect of the present disclosure provides a method of treating or preventing conditions or diseases associated with enzymatic activity of PKMYT1.

Another aspect of the present disclosure provides a medical use for preventing conditions or diseases associated with enzymatic activity of PKMYT1.

Another aspect provides methods of using compounds according to Formula I, or a pharmaceutically acceptable salt thereof, for the treatment of cancer.

A further aspect provides a pharmaceutical composition comprising a compound of the disclosure and an excipient and/or pharmaceutically acceptable carrier.

A further aspect provides a pharmaceutical composition comprising a compound of the disclosure and an excipient and/or pharmaceutically acceptable carrier for treating or preventing conditions or diseases associated with enzymatic activity of PKMYT1.

A further aspect provides processes for making compounds of Formula I.

These and other aspects and embodiments are described below.

### DETAILED DESCRIPTION OF THE INVENTION

### Abbreviations and Definitions

The following abbreviations and terms have the indicated meanings throughout:

| Abbreviation | Meaning |
|---|---|
| Ac₂O | Acetic anhydride |
| anhyd | Anhydrous |
| Aq | Aqueous |
| Ar | Argon |
| Boc | Tert-butoxycarbonyl |
| ° C. | Degrees Celsius |
| calcd | Calculated |
| chiral SFC | Chiral Supercritical fluid chromatography |
| d | Doublet |
| dd | Doublet of doublets |
| ddd | Doublet of doublets of doublets |
| dt | Doublet of triplets |
| DCM | Dichloromethane |
| DIEA | N,N-Diisopropylethylamine |
| DMB-NH₂ | (2,4-dimethoxyphenyl)methanamine |
| DME | Dimethoxyethane |
| DMF | N,N-Dimethylformamide |
| DMSO | Dimethyl sulfoxide |
| DPPA | Diphenylphosphoryl azide |
| dppp | 1,3-Bis(diphenylphosphino)propane |
| eq or equiv | Equivalent |
| EtOAc | Ethyl acetate |
| EtOH | Ethanol |
| FA or FAC | Formic acid |
| FCC | Flash column chromatography |
| g | Gram(s) |
| h or hr | Hour(s) |
| HATU | 1-[Bis(dimethylamino)methylene]-1H-1,2,3- |
| | triazolo[4,5-b]pyridinium 3-oxide |
| | hexafluorophosphate; Hexafluorophosphate |
| | Azabenzotriazole Tetramethyl Uronium |
| HOAc | Acetic acid |
| HPLC | High pressure liquid chromatography |
| H₂ | Hydrogen |
| L | Liter(s) |
| M | Molar or molarity |
| m | Multiplet |
| MeCN | Acetonitrile |
| MeOH | Methanol |
| MHz | Megahertz (frequency) |
| Min | Minute(s) |
| mL | Milliliter(s) |
| m/z | Mass to charge ratio |
| μL | Microliter(s) |
| mol | Mole(s) |
| mmol | Millimole(s) |
| MS | Mass spectral analysis |
| N₂ | Nitrogen |
| N | Normal or normality |
| NBS | N-bromosuccinimide |
| NIS | N-iodosuccinimide |
| nM | Nanomolar |
| NMR | Nuclear magnetic resonance spectroscopy |
| Ph₂O | Diphenyl ether |
| Pd/C | Palladium on carbon |
| Pd/L | [2-(2-Aminophenyl)phenyl]- |
| | methylsulfonyloxy-palladium dicyclohexyl-[2- |
| | (2,6-dimethoxyphenyl)phenyl]phosphane |
| Pd₂(dba)₃ | Tris(dibenzylideneacetone)dipalladium(0) |
| Pd(dppf)Cl₂•DCM | [1,1′-Bis(diphenyl- |
| | phosphino)ferrocene]dichloropalladium(II), |
| | complex with dichloromethane |
| Py | Pyridine |
| Q | Quartet |
| RT | Room temperature |
| s | Singlet |
| soln | Solution |
| SPhos | Dicyclohexyl(2′,6′-dimethoxy-[1,1′-biphenyl]- |
| | 2-yl)phosphane |
| SphosPdG3 | (2-Dicyclohexylphosphino-2′,6′- |
| | dimethoxybiphenyl) [2-(2′-amino-1,1′- |
| | biphenyl)]palladium(II) methanesulfonate |
| t or tr | Triplet |
| TFA | Trifluoroacetic acid |
| t-BuOH | Tert-butyl alcohol |
| TMSCl | Chloro(trimethyl)silane |
| THF | Tetrahydrofuran |

The symbol “-” represents a single bond, and “=” represents a double bond. The symbol [symbol] represents a single bond, a double bond, or a delocalized a bond.

As used herein, the singular forms “a,” “an,” and “the” include plural references unless the context clearly dictates otherwise.

When a variable is defined generically, with a number of possible substituents, each individual radical can be defined with or without the bond. For example, if R¹ can be hydrogen, this can be indicated as “—H” or “H” in the definition of R¹.

When chemical structures are depicted or described, unless explicitly stated otherwise, all carbons are assumed to have hydrogen substitution to conform to a valence of four. For example, in the structure on the left-hand side of the schematic below, there are nine hydrogens implied. The nine hydrogens are depicted in the right-hand structure. Sometimes a particular atom in a structure is described in textual formula as having a hydrogen or hydrogens as substitution (expressly defined hydrogen), for example, —CH₂CH₂—. It is understood by one of ordinary skill in the art that the aforementioned descriptive expressions are common in the chemical arts to provide brevity and simplicity to description of otherwise complex structures.

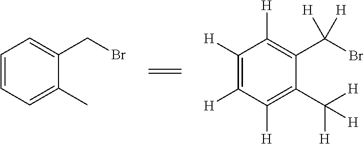

If a group “R” is depicted as “floating” on a ring system, as for example in the formula:

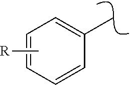

then, unless otherwise defined, a substituent “R” may reside on any atom of the ring system, assuming replacement of a depicted, implied, or expressly defined hydrogen from one of the ring atoms, so long as a stable structure is formed.

“Halogen” or “halo” refers to fluorine, chlorine, bromine, or iodine.

The term “Cₙ₋ₘ” or “Cₙ-Cₘ” indicates a range which includes the endpoints, wherein n and m are integers and indicate the number of carbons. Examples include C₁₋₄, C₁-C₄. C₁₋₆, C₁-C₆, and the like.

“Alkyl” refers to a branched or straight hydrocarbon chain having from 1 to 10 carbon atoms, 1 to 8 carbon atoms, 1 to 6 carbon atoms, 1 to 4 carbon atoms, or 1 to 2 carbon atoms, such as methyl, ethyl, n-propyl, isopropyl, n-butyl, isobutyl, sec-butyl, t-butyl, pentyl, hexyl, and heptyl. The term “Cₙ₋ₘ alkyl” or (Cₙ-Cₘ) alkyl, refers to an alkyl group having n to m carbon atoms. In some embodiments, alkyl refers to (C₁-C₆)alkyl.

“Alkylene” refers to an optionally substituted bivalent saturated aliphatic radical having from 1 to 10 carbon atoms, 1 to 8 carbon atoms, 1 to 6 carbon atoms, 1 to 4 carbon atoms, or 1 to 2 carbon atoms. In some embodiments, the alkylene group is unsubstituted or not optionally substituted. The term “Cₙ₋ₘ alkylene” refers to an alkylene group having n to m carbon atoms. Examples of alkylene groups include, but are not limited to, methylene, ethan-1,2-diyl, propan-1,3-diyl, propan-1,2-diyl, butan-1,4-diyl, butan-1,3-diyl, butan-1,2-diyl, 2-methyl-propan-1,3-diyl and the like.

“Alkenyl” refers to an alkyl moiety as defined herein having at least one carbon-carbon double bond.

“Alkoxy” refers to a moiety of the formula —OR′, wherein R′ is an (C₁-C₆)alkyl moiety as defined herein. The term “Cₙ₋ₘ alkoxy” or (Cₙ-Cₘ) alkoxy refers to an alkoxy group, the alkyl group of which has n to m carbons. Examples of alkoxy moieties include, but are not limited to, methoxy, ethoxy, isopropoxy, and the like.

“Acyl” refers to a moiety of the formula —C(O)R′, wherein R′ is an (C₁-C₆)alkyl moiety as defined herein. The term “Cₙ₋ₘ acyl” or (Cₙ-Cₘ) acyl refers to an acyl group, having a total of n to m carbons. Examples of alkoxy moieties include, but are not limited to, acetyl, propionyl, butyryl, and the like.

The term “amino” refers to a group of formula —NH₂.

“Aryl” means a monovalent six- to fourteen-membered, mono-, bi, or tri-carbocyclic ring (e.g., having two or three fused rings), wherein the monocyclic ring is aromatic and at least one of the rings in the bi- or tri-cyclic ring is aromatic. The term “Cₙ₋ₘ aryl” or “(Cₙ-Cₘ) aryl” refers to an aryl group having from n to m ring carbon atoms. In some embodiments, aryl groups have from 6 to about 10 carbon atoms. In some embodiments, aryl groups have 6 carbon atoms. In some embodiments, aryl groups have 10 carbon atoms. Unless stated otherwise, the point of attachment of the group may be located on any atom of any ring within the radical, valency rules permitting. Representative examples include phenyl, naphthyl, indanyl, and the like.

“Carbocyclyl” refers to a saturated, partially unsaturated, or aromatic ring having 3 to 14 carbon atoms. The carbocyclyl can be a 3 to 7 membered monocycle, 6 to 12 membered bicycle or polycycle. The bicycle or polycycle can be a bridged-ring, fused-ring, or spiro-ring system.

“Cycloalkyl” refers to a non-aromatic hydrocarbon ring system (monocyclic, bicyclic, or polycyclic), including cyclized alkyl and alkenyl groups. The term “Cₙ₋ₘ cycloalkyl” or “(Cₙ-Cₘ) cycloalkyl” refers to a cycloalkyl that has n to m ring member carbon atoms. Cycloalkyl groups can include mono- or polycyclic (e.g., having 2, 3, or 4 fused rings) groups and spirocycles. Cycloalkyl groups can have 3, 4, 5, 6, 7, 8, 9, 10, 11, 12, 13, or 14 ring-forming carbons (C₃₋₁₄). In some embodiments, the cycloalkyl group has 3 to 14 members, 3 to 10 members, 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, the cycloalkyl group is a C₃₋₁₄ cycloalkyl group. In some embodiments, the cycloalkyl group is a C₃₋₆ monocyclic cycloalkyl group. Ring-forming carbon atoms of a cycloalkyl group can be optionally oxidized to form an oxo group. Cycloalkyl groups also include cycloalkylidenes. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcaranyl, bicyclo[1.1.1]pentanyl, bicyclo[2.1.1]hexanyl, and the like. In some embodiments, cycloalkyl includes a single saturated carbocyclic ring of three to eight ring carbons, such as cyclopropyl, cyclobutyl, cyclopentyl, cyclohexyl, cycloheptyl, or cyclooctyl. In some embodiments, the cycloalkyl group is cyclopropyl, cyclobutyl, cyclopentyl, or cyclohexyl.

A cycloalkyl group can be unsubstituted or optionally substituted. When optionally substituted, one or more hydrogen atoms of the cycloalkyl group (e.g., from 1 to 4, from 1 to 2, or 1) may be replaced with a moiety as described. In some embodiments, a substituted cycloalkyl group can incorporate an exo- or endocyclic alkene (e.g., cyclohex-2-en-1-yl).

The term “cyano” or “nitrile” refers to a group of formula —C═N, which also may be written as —CN or CN.

The term “heteroatom” used herein is meant to include boron, phosphorus, sulfur, oxygen, and nitrogen.

The term “haloalkyl” as used herein refers to an alkyl group in which one or more of the hydrogen atoms has been replaced by a halogen atom. The term “Cₙ₋ₘ haloalkyl” or (Cₙ-Cₘ) haloalkyl refers to a Cₙ₋ₘ alkyl group having n to m carbon atoms and from at least one up to {2(n to m)+1}halogen atoms, which may either be the same or different. In some embodiments, the haloalkyl group has 1 to 6 or 1 to 4 carbon atoms. In some embodiments, the halogen atoms include fluoro atoms. In some embodiments, the haloalkyl group is a fluoroalkyl group. Example haloalkyl groups include CF₃, C₂F₅, CHF₂, CCl₃, CHCl₂, C₂Cl₅, and the like.

The term “haloalkoxy” refers to a group of formula —O-haloalkyl, wherein the haloalkyl group is as defined above. The term “Cₙ₋ₘ haloalkoxy” or (Cₙ-Cₘ) haloalkoxy refers to a haloalkoxy group, the haloalkyl group of which has n to m carbons. In some embodiments, the haloalkoxy group has 1 to 6, 1 to 4, or 1 to 3 carbon atoms. Example haloalkoxy groups include trifluoromethoxy and the like.

“Heteroaryl” means a monocyclic, fused bicyclic, or fused tricyclic, monovalent radical of 5 to 14 ring atoms containing one or more (e.g., one, two, three, or four) ring members independently selected from —O—, —S(O)ₙ— (n is 0, 1, or 2), —N—, and —N(R′)—, and the remaining ring atoms being carbon, wherein the ring comprising a monocyclic radical is aromatic and wherein at least one of the fused rings comprising a bicyclic or tricyclic radical is aromatic. One or two ring carbon atoms of any nonaromatic rings comprising a bicyclic or tricyclic radical may be replaced by a —C(O)—, —C(S)—, or —C(═NH)— group. R′ is hydrogen, alkyl, hydroxy, alkoxy, acyl, or alkylsulfonyl. Unless stated otherwise, the point of attachment may be located on any atom of any ring of the heteroaryl group, valency rules permitting. In particular, when the point of attachment is located on the nitrogen, an additional nitrogen substituent is not present. More specifically, the term heteroaryl includes, but is not limited to, 1,2,4-triazolyl, 1,3,5-triazolyl, phthalimidyl, pyridinyl, pyrrolyl, imidazolyl, thienyl, furanyl, indolyl, isoindolyl, indolinyl (including, for example, 2,3-dihydro-1H-indol-2-yl or 2,3-dihydro-1H-indol-5-yl, and the like), isoindolinyl, benzimidazolyl, benzofuranyl, cinnolinyl, indolizinyl, naphthyridinyl, phthalazinyl, pyridazopyrazinyl, pteridinyl, purinyl, quinazolinyl, quinoxalinyl, tetrazoyl, pyrazolyl, pyrazinyl, pyrimidinyl, pyridazinyl, oxazolyl, isooxazolyl, oxadiazolyl, benzoxazolyl, quinolinyl, isoquinolinyl, tetrahvdroisoquinolinyl (including, for example, tetrahydroisoquinolin-4-yl or tetrahydroisoquinolin-6-yl, and the like), pyrrolo[3,2-c]pyridinyl (including, for example, pyrrolo[3,2-c]pyridin-2-yl or pyrrolo[3,2-c]pyridin-7-yl, and the like), pyrazolopyridinyl, tetrahydropyrazolopyridinyl, benzopyranyl, thiazolyl, isothiazolyl, thiadiazolyl, benzothiazolyl, benzothienyl, and derivatives thereof, including N-oxides and protected derivatives thereof.

As used herein, “heterocyclyl” refers to a saturated, partially unsaturated, or aromatic ring or ring system, which has at least one heteroatom ring member independently selected from boron, nitrogen, sulfur, oxygen, and phosphorus, and which has 4-14 ring members, 4-10 ring members, 4-7 ring members, or 4-6 ring members. Heterocyclyl groups can include mono- or bicyclic or polycyclic (e.g., having two or three fused or bridged rings) ring systems or spirocycles. In some embodiments, the heterocyclyl group is a monocyclic group having 1, 2, or 3 heteroatoms independently selected from nitrogen, sulfur, and oxygen. Ring-forming carbon atoms and heteroatoms of a heterocyclyl group can be optionally oxidized to form an oxo or sulfonyl group or other oxidized linkage (e.g., C(O), S(O), C(S), S(O)₂, N-oxide, and the like) or a nitrogen atom can be quaternized. In some embodiments, the heterocyclyl group is an aromatic monocyclic or bicyclic group having 1, 2, or 3 heteroatoms independently selected from nitrogen, sulfur, and oxygen. Example of heterocyclyl groups include pyridinyl, naphthyridinyl, pyridazopyrazinyl, pyridopyrazinyl, quinolinyl, quinoxalinyl, etc.

The term “hydroxyl” or “hydroxy” refers to an —OH moiety.

“Hydroxyalkyl” means an alkyl group, as defined herein, substituted with at least one, particularly, 1, 2, 3, or 4, hydroxy groups. The term “Cₙ₋ₘ hydroxyalkyl” or (Cₙ-Cₘ) hydroxyalkyl refers to a hydroxyalkyl group, the hydroxyalkyl group of which has n to m carbons. In some embodiments, the hydroxyalkyl group has 1 to 6, 1 to 4, or 1 to 3 carbon atoms.

The term “oxo” refers to an oxygen atom as a divalent substituent, forming a carbonyl group when attached to carbon, or attached to a heteroatom forming a sulfoxide or sulfone group, or an N-oxide group. In some embodiments, heterocyclic groups may be optionally substituted by 1 or 2 oxo (=O) substituents.

As used herein, a “leaving group” (Lv) is an art-understood term referring to a molecular fragment that departs with a pair of electrons in heterolytic bond cleavage, wherein the molecular fragment is an anion or neutral molecule. As used herein, a leaving group can be an atom or a group capable of being displaced by a nucleophile. See, for example, Smith, March Advanced Organic Chemistry 6^{th} ed. (501-502). Exemplary leaving groups include, but are not limited to, halo (e.g., chloro, bromo, iodo), tosyl, mesyl, and besyl. In certain embodiments, the leaving group is a halogen.

“Subject” for the purposes of the present disclosure includes humans and any other animals, particularly mammals, and other organisms. Thus, the methods are applicable to both human therapy and veterinary applications. In some embodiments, the subject is a human or other mammal. Examples of other mammals include mice, rats, other rodents, rabbits, dogs, cats, swine, cattle, sheep, horses, and non-human primates.

“Therapeutically effective amount” is an amount of a compound as described herein that, when administered to a patient, ameliorates a symptom of the disease. The amount of a compound which constitutes a “therapeutically effective amount” will vary depending on the compound, the disease state and its severity, the age of the patient to be treated, and the like.

“Cancer” refers to cellular-proliferative disease states, including carcinomas, sarcomas, leukemias, and lymphomas. The term “cancerous cell” as provided herein, includes a cell afflicted by any one of the above-identified conditions.

“Pharmaceutically acceptable salts” includes “pharmaceutically acceptable acid addition salts” and “pharmaceutically acceptable base addition salts.” “Pharmaceutically acceptable acid addition salts” refers to those salts that retain the biological effectiveness of the free bases and that are not biologically or otherwise undesirable, formed with inorganic acids as well as organic acids.

“Pharmaceutically acceptable base addition salts” include those derived from inorganic bases and organic bases.

The term. “compound,” as used herein is meant to include all stereoisomers, geometric isomers, tautomers, and isotopes of the structures depicted. The term is also meant to refer to compounds of the inventions, regardless of how they are prepared, e.g., synthetically, through biological process (e.g., metabolism or enzyme conversion), or a combination thereof.

Compounds as described herein can also include all isotopes of atoms occurring in synthetic intermediates or final compounds. Isotopes include those atoms having the same atomic number but different mass numbers. For example, isotopes of hydrogen include tritium and deuterium.

Any one of the process steps or sequences disclosed and/or claimed herein can be performed under an inert gas atmosphere, more particularly under argon or nitrogen. In addition, the methods of the present disclosure may be carried out as semi-continuous or continuous processes.

In general, the nomenclature used in this Application is based on naming conventions adopted by the International Union of Pure and Applied Chemistry (IUPAC). Chemical structures shown herein were prepared using CHEMDRAW®. Any open valency appearing on a carbon, oxygen, or nitrogen atom in the structures herein indicates the presence of a hydrogen atom.

### Compounds of the Disclosure

One aspect of the present disclosure provides a compound according to Formula I:

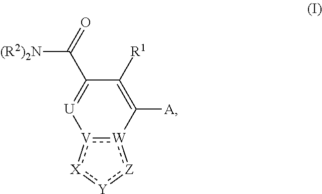

or a pharmaceutically acceptable salt thereof,
wherein:
each [symbol] represents a single bond, a double bond, or a delocalized π bond;
U is selected from the group consisting of N and CH;
V and W are independently selected from the group consisting of N and C;
X is selected from the group consisting of CR³, N, NR^{3a}, S, and O;
Y is selected from the group consisting of CR⁴, N. NR^{4a}, S, and O;
Z is selected from the group consisting of CR⁵, N. NR^{a}, S, and O;
ring A is
3- to 14-membered carbocyclyl substituted with 1, 2, 3, 4, or 5 R⁶, or
5- to 14-membered heterocyclyl comprising at least one oxygen atom, nitrogen atom, or sulfur atom, wherein the heterocyclyl is substituted with 0, 1, 2, 3, or 4 R⁶;
R¹ is selected from the group consisting of amino, hydroxy, and hydrogen;
each R² is independently selected from the group consisting of hydrogen and C₁₋₆ alkyl;
R³, R⁴, and R⁵ are independently selected from the group consisting of hydrogen, amino, hydroxy, halogen, cyano, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, —NO₂, —CO(C₁₋₆ alkyl), —COOH, —COO(C₁₋₆ alkyl), —CON(R⁷)₂, -L-(5- to 12-membered heterocyclyl), -L-(5- to 14-membered heteroaryl, and -L-(C₆₋₁₄ aryl), wherein the 5- to 12-membered heterocyclyl, 5- to 14-membered heteroaryl, and C₆₋₁₄ aryl are independently substituted with 0, 1, 2, 3, or 4 R⁶;
each L is independently selected from the group consisting of a covalent bond, —O—, C₁₋₆ alkylene, and NR^{a};
any two adjacent R³, R⁴, and R⁵ are optionally taken together with the carbon atoms to which they are attached to form a 5- or 6-membered carbocyle or heterocycle, each of which is optionally substituted with 0, 1, 2, 3, or 4 R⁶;
R^{3a}, R^{4a}, R^{5a}, and R⁷ are independently selected from the group consisting of hydrogen, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, -L-(5- to 12-membered heterocyclyl), L-(5- to 14-membered heteroaryl, and -L-(C₆₋₁₄ aryl), wherein the C₃₋₈ cycloalkyl, 5- to 12-membered heterocyclyl, 5- to 14-membered heteroaryl, and C₆₋₁₄ aryl are independently substituted with 0, 1, 2, 3, or 4 R⁶;
each R⁶ is independently selected from the group consisting of hydroxy, amino, cyano, halogen, C₁₋₆ alkyl, C₁₋₆ haloalkyl, C₁₋₆ hydroxyalkyl, C₁₋₆ alkoxy, oxo, —NO₂, —CHO, —CO(C₁₋₆ alkyl). —COOR^{a}. —CON(R^{a})₂, —SO₂(C₁₋₆ alkyl), and —SO₂N(R^{a})₂; and
each R^{a} is independently selected from the group consisting of hydrogen, C₁₋₆ alkyl, and C₁₋₆ haloalkyl.

In some embodiments, ring A is substituted with at least one R⁶ that is selected from the group consisting of hydroxy, amino, cyano, C₁₋₆ alkyl, C₁₋₆ haloalkyl. C₁₋₆ hydroxyalkyl, oxo, —NO₂, —CHO, —CO(C₁₋₆ alkyl), —COOR^{a}, —CON(R^{a})₂, and —SO₂N(R^{a})₂ when ring A is a carbocycle and R¹ and each R² are hydrogen.

In some embodiments, ring A is substituted with at least one R⁶ that is selected from the group consisting of hydroxy, amino, cyano, halogen, C₁₋₆ alkyl, C₁₋₆ haloalkyl, C₁₋₆ hydroxyalkyl, oxo, —NO₂. —CHO. —CO(C₁₋₆ alkyl). —COOR, —CON(R^{a})₂, and —SO₂N(R^{a})₂ when ring A is a carbocycle, R¹ and each R² are hydrogen, and any two adjacent R³, R⁴, and R⁵ form a 5- or 6-membered carbocyle or heterocycle.

In some embodiments, ring A is substituted with at least one R⁶ that is hydroxy when ring A is a carbocycle and R¹ and each R² are hydrogen.

In some embodiments, ring A is substituted with at least one R⁶ that is selected from the group consisting of hydroxy, amino, cyano, halogen, C₁₋₆ haloalkyl, C₁₋₆ hydroxyalkyl, oxo, —NO₂, —CHO, —CO(C₁₋₆ alkyl), —COOR^{a}, —CON(R^{a})₂, —SO₂(C₁₋₆ alkyl), and —SO₂N(R^{a})₂ when ring A is a heterocycle and R¹ and each R² are hydrogen.

In some embodiments, ring A is substituted with at least one R⁶ that is selected from the group consisting of hydroxy, amino, cyano, halogen, C₁₋₆ haloalkyl, C₁₋₆ hydroxyalkyl, C₁₋₆ alkoxy, oxo, —NO₂, —CHO, —CO(C₁₋₆ alkyl), —COOR^{a}, —CON(R^{a})₂, —SO₂(C₁₋₆ alkyl), and —SO₂N(R^{a})₂ when ring A is a bicyclic heterocycle and R¹ and each R² are hydrogen.

In some embodiments, ring A is substituted with at least one R⁶ that is selected from the group consisting of hydroxy, amino, cyano, halogen, C₁₋₆ alkyl, C₁₋₆ haloalkyl, C₁₋₆ hydroxyalkyl, oxo, —NO₂, —CHO, —CO(C₁₋₆ alkyl), —COOR^{a}, —CON(R^{a})₂, —SO₂(C₁₋₆ alkyl), and —SO₂N(R^{a})₂ when ring A is a monocyclic heterocycle and R¹ and each R² are hydrogen.

In some embodiments, compounds of Formula I and pharmaceutically acceptable salts thereof are provided wherein R¹ is amino.

In some embodiments, compounds of Formula I and pharmaceutically acceptable salts thereof are provided wherein R¹ is hydrogen.

In some embodiments, compounds of Formula I and pharmaceutically acceptable salts thereof are provided wherein R¹ is hydroxy.

In some embodiments, compounds of Formula I and pharmaceutically acceptable salts thereof are provided wherein each R² is hydrogen.

In some embodiments, compounds of Formula I and pharmaceutically acceptable salts thereof are provided wherein U is N, V is C, and W is C.

Some embodiments provide compounds having a structure according to Formula Ia:

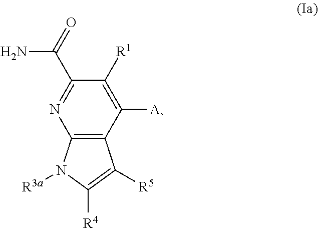

and pharmaceutically acceptable salts thereof. In some embodiments, R¹ in Formula Ia is hydrogen. In some embodiments, R¹ in Formula Ia is hydrogen and R⁵ in Formula Ia is cyano. In some such embodiments, R^{3a} is C₁₋₆ alkyl (e.g., methyl, ethyl, n-propyl, isopropyl, n-butyl, isobutyl, sec-butyl, tert-butyl, n-pentyl, branched pentyl, n-hexyl, branched hexyl, or the like) or C₁₋₆ haloalkyl (e.g., chloromethyl, dichloromethyl, trichloromethyl, fluoromethyl, difluoromethyl, trifluoromethyl, 2,2,2-trichloroethyl, 2,2,2-trifluoroethyl, pentachloroethyl, pentafluoroethyl, 1,1,1,3,3,3-hexachloropropyl, 1,1,1,3,3,3-hexafluoropropyl, or the like). In some such embodiments, R⁴ is hydrogen or C₁₋₆ alkyl. In some embodiments, R¹ in Formula Ia is hydrogen, R^{3a} is C₁₋₆ alkyl or C₁₋₆ haloalkyl, R⁴ is hydrogen, and R⁵ is cyano. In some embodiments, R¹ in Formula Ia is hydrogen, R^{3a} is C₁₋₆ alkyl or C₁₋₆ haloalkyl, R⁴ is hydrogen, R⁵ is cyano, and ring A is 3-hydroxy-2-methylphenyl or 3-hydroxy-2,6-dimethylphenyl.

In some embodiments, R¹ in Formula Ia is amino. In some embodiments, R¹ in Formula 1a is amino, and R⁵ in Formula 1a is cyano. In some such embodiments. R^{3a} is C₁₋₆ alkyl or C₁₋₆ haloalkyl. In some such embodiments, R⁴ is hydrogen or C₁₋₆ alkyl. In some embodiments, R¹ in Formula Ia is amino, R^{3a} is C₁₋₆ alkyl, R⁴ is hydrogen, and R⁵ is cyano. In some embodiments, R¹ in Formula Ia is amino, R^{3a} is C₁₋₆ alkyl, R⁴ is hydrogen, R⁵ is cyano, and ring A is 3-hydroxy-2-methylphenyl or 3-hydroxy-2,6-dimethylphenyl.

In some embodiments, the compound of Formula La is selected from any combination of compounds 4, 7, 8, 9, 12, 13, 14, 21, 22, 23, 24, 26, 28, 29, 30, 66, and 67 as set forth in Table 1. In some embodiments, the compound of Formula La is selected from any combination of compounds 5, 6, 10, 11, 25, 27, 31, 32, 33, 34, 68, 69, 71, 76, 78, 79, 80, 82, 88, 89, 90, 91, 92, 93, 96, and 97 as set forth in Table 1.

Some embodiments provide compounds having a structure according to Formula Ib:

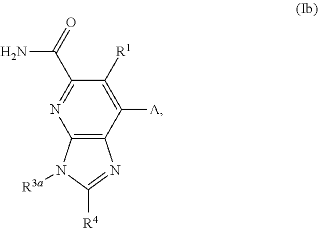

and pharmaceutically acceptable salts thereof. In some embodiments. R¹ in Formula Ib is hydrogen. In some such embodiments, R^{3a} is C₁₋₆ alkyl (e.g., methyl, ethyl, n-propyl, isopropyl, n-butyl, isobutyl, sec-butyl, tert-butyl, n-pentyl, branched pentyl, n-hexyl, branched hexyl, or the like) C₁₋₆ haloalkyl (e.g., chloromethyl, dichloromethyl, trichloromethyl, fluoromethyl, difluoromethyl, trifluoromethyl, 2,2,2-trichloroethyl, 2,2,2-trifluoroethyl, pentachloroethyl, pentafluoroethyl, 1,1,1,3,3,3-hexachloropropyl, 1,1,1,3,3,3-hexafluoropropyl, or the like), or C₃₋₈ cycloalkyl (e.g., cyclopropyl, cyclobutyl, or like, each of which is optionally substituted with 1-3 R⁶). In some such embodiments, R⁴ is hydrogen or C₁₋₆ alkyl.

In some embodiments, R¹ in Formula Ib is amino. In some such embodiments, R^{3a} is C₁₋₆ alkyl, C₁₋₆ haloalkyl, unsubstituted C₃₋₈ cycloalkyl, or halogen-substituted C₃₋₈ cycloalkyl. In some such embodiments, R⁴ is hydrogen or C₁₋₆ alkyl.

In some embodiments, the compound according to Formula Ib is compound 1 as set forth in Table 1. In some embodiments, the compound according to Formula Ib is selected from any combination of compounds 2, 3, 72, 75, 77, 81, 83, 84, and 86 as set forth in Table 1.

Some embodiments provide compounds having a structure according to Formula Ic:

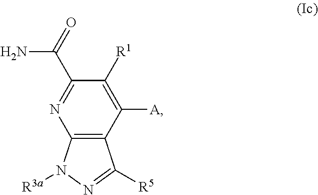

and pharmaceutically acceptable salts thereof.

In some embodiments, the compound according to Formula Ic is selected from any combination of compounds 17, 54, 55, 56, 57, and 73 as set forth in Table 1. In some embodiments, the compound according to Formula Ic is selected from any combination of compounds 58, 59, and 74 as set forth in Table 1.

Some embodiments provide compounds having a structure according to Formula Id:

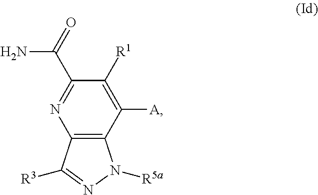

and pharmaceutically acceptable salts thereof.

In some embodiments, the compound according to Formula Id is selected from any combination of compounds 16 and 18 as set forth in Table 1.

In some embodiments, compounds of Formula I and pharmaceutically acceptable salts thereof are provided wherein U is C, V is C, and W is C.

Some embodiments provide compounds having a structure according to Formula IIa:

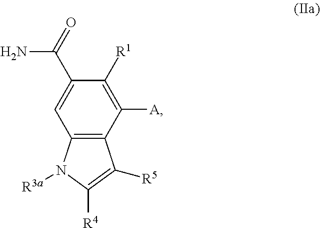

and pharmaceutically acceptable salts thereof. In some embodiments, R¹ in Formula IIa is hydrogen. In some embodiments, R¹ in Formula IIa is hydrogen and R⁵ in Formula IIa is cyano. In some such embodiments, R^{3a} is C₁₋₆ alkyl (e.g., methyl, ethyl, n-propyl, isopropyl, n-butyl, isobutyl, sec-butyl, tert-butyl, n-pentyl, branched pentyl, n-hexyl, branched hexyl, or the like) or C₁₋₆ haloalkyl (e.g., chloromethyl, dichloromethyl, trichloromethyl, fluoromethyl, difluoromethyl, trifluoromethyl, 2,2,2-trichloroethyl, 2,2,2-trifluoroethyl, pentachloroethyl, pentafluoroethyl, 1,1,1,3,3,3-hexachloropropyl, 1,1,1,3,3,3-hexafluoropropyl, or the like). In some such embodiments, R⁴ is hydrogen or C₁₋₆ alkyl.

In some embodiments, R¹ in Formula IIa is amino. In some embodiments, R¹ in Formula IIa is amino, and R⁵ in Formula IIa is cyano. In some such embodiments, R^{3a} is C₁₋₆ alkyl or C₁₋₆ haloalkyl. In some such embodiments, R⁴ is hydrogen or C₁₋₆ alkyl.

In some embodiments, the compound of Formula IIa is selected from any combination of compounds 61, 64, and 65 as set forth in Table 1. In some embodiments, the compound of Formula IIa is compound 62 or compound 63 as set forth in Table 1.

Some embodiments provide compounds having a structure according to Formula IIb:

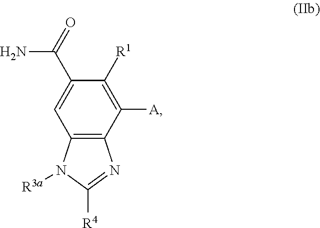

and pharmaceutically acceptable salts thereof. In some embodiments, R¹ in Formula IIb is hydrogen. In some embodiments, R¹ in Formula IIb is hydrogen and R^{3a} is C₁₋₆ alkyl (e.g., methyl, ethyl, n-propyl, isopropyl, n-butyl, isobutyl, sec-butyl, tert-butyl, n-pentyl, branched pentyl, n-hexyl, branched hexyl, or the like) or C₁₋₆ haloalkyl (e.g., chloromethyl, dichloromethyl, trichloromethyl, fluoromethyl, difluoromethyl, trifluoromethyl, 2,2,2-trichloroethyl, 2,2,2-trifluoroethyl, pentachloroethyl, pentafluoroethyl, 1,1,1,3,3,3-hexachloropropyl, 1,1,1,3,3,3-hexafluoropropyl, or the like). In some such embodiments, R⁴ is hydrogen, C₁₋₆ alkyl, or C₁₋₆ haloalkyl. In some embodiments, R¹ in Formula IIb is hydrogen, R^{3a} is C₁₋₆ haloalkyl, and R⁴ is C₁₋₆ alkyl. In some embodiments, R¹ in Formula IIb is amino. In some embodiments, R¹ in Formula IIb is amino, and R^{3a} is C₁₋₆ alkyl or C₁₋₆ haloalkyl. In some such embodiments, R⁴ is hydrogen, C₁₋₆ alkyl, or C₁₋₆ haloalkyl. In some embodiments, R¹ in Formula IIb is amino, R^{3a} is C₁₋₆ haloalkyl, and R⁴ is hydrogen or C₁₋₆ haloalkyl. In some embodiments, R¹ in Formula IIb is amino, R^{3a} is C₁₋₆ haloalkyl, and R⁴ is hydrogen. In some embodiments, R¹ in Formula IIb is amino. R^{3a} is C₁₋₆ haloalkyl. R⁴ is hydrogen, and ring A is 3-hydroxy-2-methylphenyl or 3-hydroxy-2,6-dimethylphenyl.

In some embodiments, the compound of Formula IIb is compound 15 as set forth in Table 1. In some embodiments, the compound of Formula IIb is selected from any combination of compounds 70, 85, 87, 94, and 95 as set forth in Table 1.

Some embodiments provide compounds having a structure according to Formula IIc:

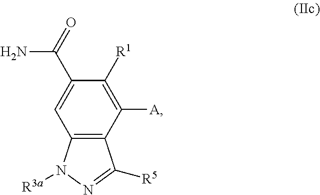

and pharmaceutically acceptable salts thereof.

In some embodiments, the compound of Formula IIc is selected from any combination of compounds 35, 36, and 60 as set forth in Table 1.

Some embodiments provide compounds having a structure according to Formula IId:

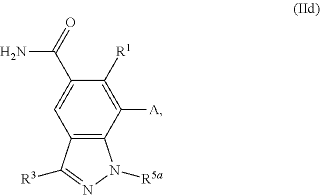

and pharmaceutically acceptable salts thereof.

In some embodiments, the compound according to Formula IId is selected from any combination of compounds 37 and 40 as set forth in Table 1.

Some embodiments provide compounds having a structure according to Formula IIe:

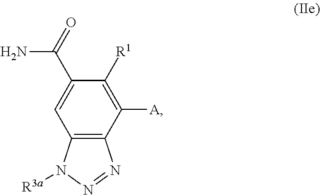

and pharmaceutically acceptable salts thereof.

In some embodiments, the compound of Formula IIe is compound 98 as set forth in Table 1.

In some embodiments, compounds of Formula I and pharmaceutically acceptable salts thereof are provided wherein U is C, V is N. and W is C.

Some embodiments provide compounds having a structure according to Formula IIIa:

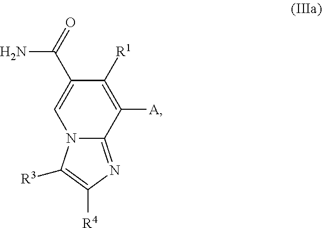

and pharmaceutically acceptable salts thereof.

In some embodiments, the compound of Formula IIIa is selected from any combination of compounds 42, 46, 47, 48, 49, and 50 as set forth in Table 1. In some embodiments, the compound of Formula IIIa is compound 44 or compound 45 as set forth in Table 1.

Some embodiments provide compounds having a structure according to Formula IIb:

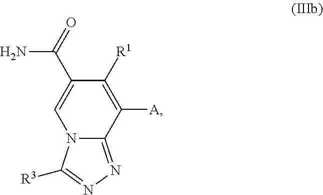

and pharmaceutically acceptable salts thereof.

In some embodiments, the compound of Formula IIIb is compound 51 as set forth in Table 1. In some embodiments, the compound of Formula IIIb is compound 52 or compound 53 as set forth in Table 1.

In some embodiments, compounds of Formula I and pharmaceutically acceptable salts thereof are provided wherein R³. R⁴, and R⁵ are independently selected from the group consisting of hydrogen, halogen, cyano. C₁₋₆ alkyl, C₁₋₆ haloalkyl, —COO(C₁₋₆ alkyl), and —CON(R⁷)₂. In some embodiments, each R⁷ is independently selected from the group consisting of C₁₋₆ alkyl and C₁₋₆ haloalkyl.

In some embodiments, ring A is selected from the group consisting of:

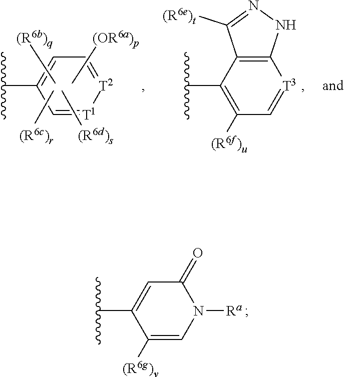

T¹ and T² are independently selected from the group consisting of N and CH;
T³ is selected from the group consisting of N and CR^{6h};
subscripts p, q, r, s, t, u, and v are independently 0 or 1;
R^{6b}, R^{6c}, R^{6d}, R^{6c}, R^{6f}, R^{6g}, and R^{6h} are independently selected from the group consisting of C₁₋₆ alkyl, C₁₋₆ alkoxy, C₁₋₆ haloalkyl, and halogen; and
R^{6a} is selected from the group consisting of hydrogen and C₁₋₆ alkyl. In such embodiments, —OR^{6a} and R^{6b-h} correspond to R⁶ in Formula I.

In some embodiments, ring A in compounds according to Formula I, Ia, Ib, Ic, Id, IIa, IIb, IIc, IId, IIIa, and/or IIIb is

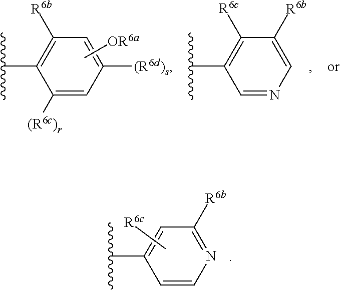

In some embodiments, ring A in compounds according to Formula I, Ia, Ib, Ic, Id, IIa, IIb, IIc, IId, IIIa, and/or IIIb is

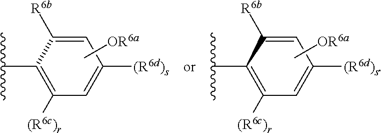

In some such embodiments, subscript r is 0 and subscript s is 0. In some embodiments, subscript r is 1 and subscript s is 0. In some embodiments, subscript r is 1 and subscript s is 1. In some embodiments, R^{6a} is hydrogen and R^{6b} is C₁₋₆ alkyl (e.g., methyl). R^{6c} and R^{6d}, when present, are also C₁₋₆ alkyl (e.g., methyl) in some embodiments.

In some embodiments, ring A in compounds according to Formula I, Ia, Ib, Ic, Id, IIa, IIb, IIc, IId, IIIa, and/or IIIb is

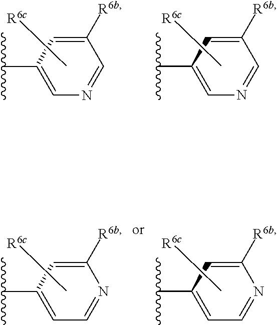

In some such embodiments, R^{6b} is halogen (e.g., chloro or fluoro) and R^{6c} is C₁₋₆ alkyl (e.g., methyl).

In some embodiments, ring A in compounds according to Formula I, Ia, Ib, Ic, Id, IIa, IIb, IIc, IId, IIIa, and/or IIIb is selected from the group consisting of

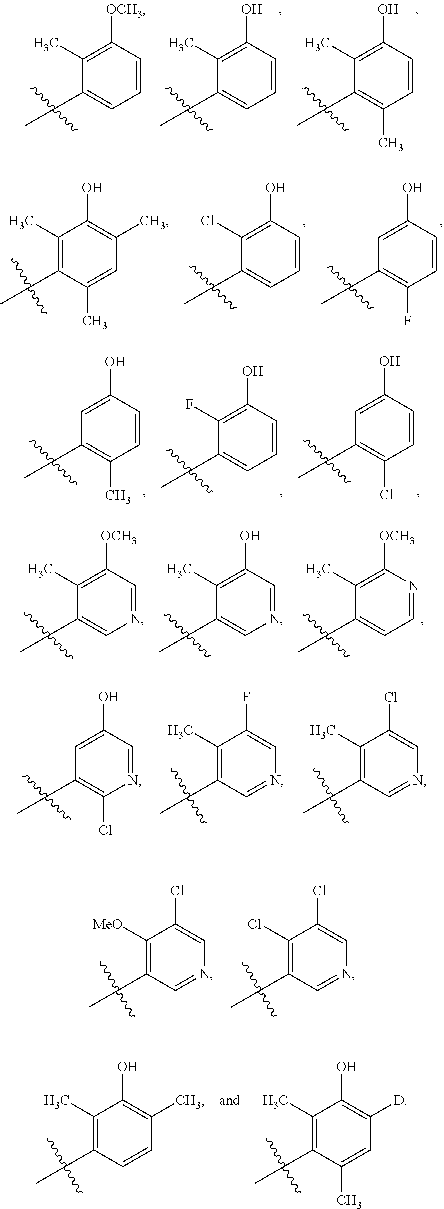

In some embodiments, ring A in compounds according to Formula I, Ia, Ib, Ic, Id, IIa, IIb. IIc. IId, IIIa, and/or IIIb is selected from the group consisting of

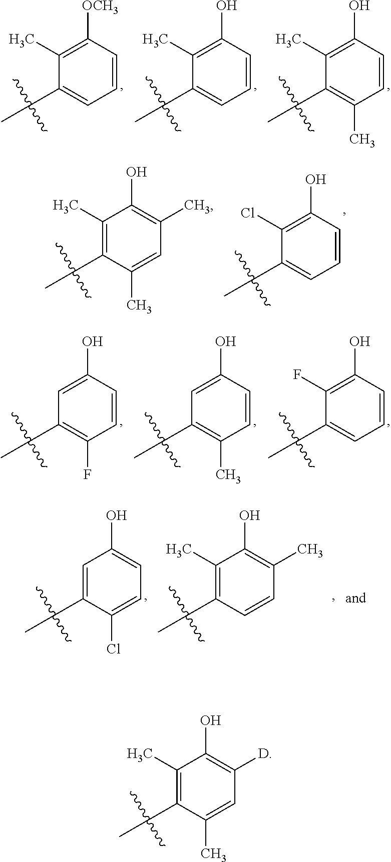

In some embodiments, ring A in compounds according to Formula I, Ia, Ib, Ic, Id, IIa, IIb, IIc, IId, IIIa, and/or IIIb is:

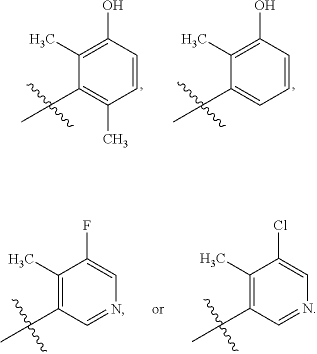

In some embodiments, ring A in compounds according to Formula I, Ia, Ib, Ic, Id, IIa, IIb, IIc, IId, IIIa, and/or IIIb is:

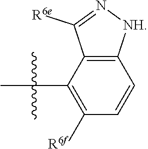

In some such embodiments, R^{6c} and R^{6f} are independently hydrogen or C₁₋₆ alkyl.

In some embodiments, ring A in compounds according to Formula I, Ia, Ib, Ic, Id, IIa, IIb, IIc, IId, IIIa, and/or IIIb is:

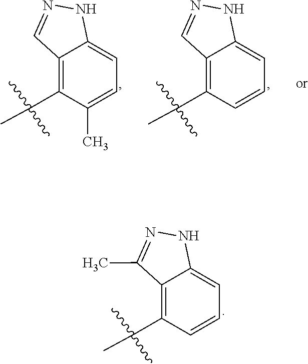 [SMILES: *c1c(C)ccc2[nH]ncc12 ; *c1cccc2[nH]ncc12 ; *c1cccc2[nH]nc(C)c12]

In some embodiments, ring A in compounds according to Formula I, Ia, Ib, Ic, Id, IIa, IIb, IIc, IId, IIIa, and/or IIIb is:

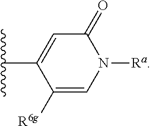

In some such embodiments, R^{6g} and R^{a} are each independently hydrogen or C₁₋₆ alkyl.

In some embodiments, ring A in compounds according to Formula I, Ia, Ib, Ic, Id, IIa, IIb, IIc, IId, IIIa, and/or IIIb is:

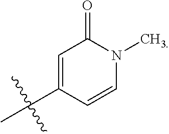 [SMILES: *c1ccn(C)c(=O)c1]

In some embodiments, compounds of Formula I and pharmaceutically acceptable salts thereof are provided wherein ring A is C₆₋₁₄ aryl substituted with two or three R⁶, wherein one R⁶ is hydroxy and one or two R⁶ are C₁₋₆ alkyl.

In some embodiments, compounds of Formula I and pharmaceutically acceptable salts thereof are provided wherein ring A is selected from the group consisting of 3-hydroxy-2,6-dimethylphenyl, 3-hydroxy-2-methylphenyl, and 5-hydroxy-2-methylphenyl.

In some embodiments, compounds of Formula I and pharmaceutically acceptable salts thereof are provided wherein ring A is 5- or 6-membered heteroaryl substituted with two R⁶, each of which is independently selected from the group consisting of halogen and C₁₋₆ alkyl.

In some embodiments, compounds of Formula I and pharmaceutically acceptable salts thereof are provided wherein ring A is selected from the group consisting of 5-chloro-4-methylpyridin-3-yl and 5-fluoro-4-methylpyridin-3-yl.

Some embodiments provide compounds having a structure according to Formula IVa:

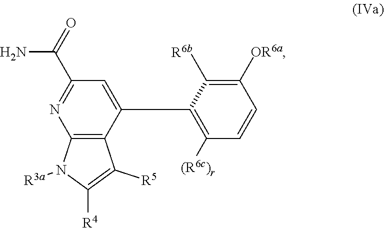

and pharmaceutically acceptable salts thereof. In some embodiments, subscript r is 0. In some embodiments, subscript r is 1. In some embodiments. R^{6a} is hydrogen and R^{6b} is C₁₋₆ alkyl (e.g., methyl, ethyl, n-propyl, isopropyl, n-butyl, isobutyl, sec-butyl, tert-butyl, n-pentyl, branched pentyl, n-hexyl, branched hexyl, or the like) in compounds of Formula IVa. R^{6c}, when present, is also C₁₋₆ alkyl (e.g., methyl) in compounds of Formula IVa.

In some embodiments, R¹ in Formula IVa is hydrogen. In some embodiments, R¹ in Formula IVa is hydrogen and R⁵ is cyano. In some such embodiments. R^{3a} is C₁₋₆ alkyl (e.g., methyl) or C₁₋₆ haloalkyl (e.g., chloromethyl, dichloromethyl, trichloromethyl, fluoromethyl, difluoromethyl, trifluoromethyl, 2,2,2-trichloroethyl, 2,2,2-trifluoroethyl, pentachloroethyl, pentafluoroethyl, 1,1,1,3,3,3-hexachloropropyl, 1,1,1,3,3,3-hexafluoropropyl, or the like). In some such embodiments, R⁴ is hydrogen or C₁₋₆ alkyl.

In some embodiments, R¹ in Formula IVa is amino. In some embodiments, R¹ in Formula IVa is amino and R⁵ in Formula IVa is cyano. In some embodiments, R^{3a} is C₁₋₆ alkyl or C₁₋₆ haloalkyl in Formula IVa. In some embodiments, R^{3a} is C₁₋₆ alkyl or C₁₋₆ haloalkyl in Formula IVa and R⁴ is hydrogen or C₁₋₆ alkyl.

Some embodiments provide compounds having a structure according to Formula IVb:

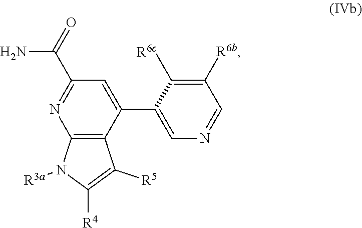

and pharmaceutically acceptable salts thereof. In some embodiments, R^{6b} is halogen (e.g., chloro or fluoro) and R^{6c} is C₁₋₆ alkyl (e.g., methyl). In some embodiments, R^{6b} and R^{6c} are each independently halogen (e.g., chloro or fluoro).

In some embodiments, R¹ in Formula IVb is hydrogen. In some embodiments, R¹ in Formula IVb is hydrogen and R⁵ is cyano. In some such embodiments, R^{3a} is C₁₋₆ alkyl or C₁₋₆ haloalkyl in Formula IVb. In some such embodiments, R^{3a} is C₁₋₆ alkyl or C₁₋₆ haloalkyl in Formula IVb and R⁴ is hydrogen or C₁₋₆ alkyl.

In some embodiments, R¹ in Formula IVb is amino. In some embodiments, R¹ in Formula IVb is amino and R⁵ in Formula IVb is cyano. In some such embodiments, R^{3a} is C₁₋₆ alkyl or C₁₋₆ haloalkyl in Formula IVb. In some such embodiments, R^{3a} is C₁₋₆ alkyl or C₁₋₆ haloalkyl in Formula IVb and R⁴ is hydrogen or C₁₋₆ alkyl.

Some embodiments provide compounds having a structure according to Formula Va:

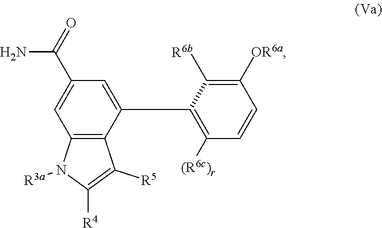

and pharmaceutically acceptable salts thereof. In some embodiments, subscript r is 0. In some embodiments, subscript r is 1. In some embodiments, R^{6a} is hydrogen and R^{6b} is C₁₋₆ alkyl (e.g., methyl) in compounds of Formula Va. R^{6c}, when present, is also C₁₋₆ alkyl (e.g., methyl) in compounds of Formula Va.

In some embodiments, R¹ in Formula Va is hydrogen. In some embodiments, R¹ in Formula Va is hydrogen and R⁵ in Formula Va is cyano. In some embodiments, R^{3a} is C₁₋₆ alkyl or C₁₋₆ haloalkyl in Formula Va. In some embodiments, R^{3a} is C₁₋₆ alkyl or C₁₋₆ haloalkyl in Formula Va and R⁴ is hydrogen or C₁₋₆ alkyl.

In some embodiments. R¹ in Formula Va is amino. In some embodiments, R¹ in Formula Va is amino, and R⁵ in Formula Ia is cyano. In some such embodiments, R^{3a} is C₁₋₅ alkyl or C₁₋₆ haloalkyl in Formula Va. In some such embodiments, R^{3a} is C₁₋₆ alkyl or C₁₋₆ haloalkyl in Formula Va and R⁴ is hydrogen or C₁₋₆ alkyl.

Some embodiments provide compounds having a structure according to Formula Vb:

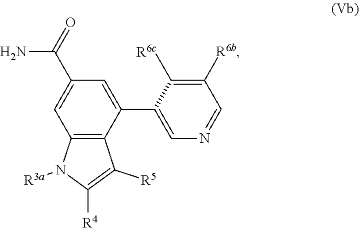

and pharmaceutically acceptable salts thereof. In some embodiments, R^{6b} is halogen (e.g., chloro or fluoro) and R^{6b} is C₁₋₆ alkyl (e.g., methyl).

In some embodiments, R¹ in Formula Vb is hydrogen. In some embodiments, R¹ in Formula Vb is hydrogen and R⁵ is cyano. In some such embodiments, R^{3a} is C₁₋₆ alkyl or C₁₋₆ haloalkyl in Formula Vb. In some such embodiments, R^{3a} is C₁₋₆ alkyl or C₁₋₆ haloalkyl in Formula Vb and R⁴ is hydrogen or C₁₋₆ alkyl.

In some embodiments, R¹ in Formula Vb is amino. In some embodiments, R¹ in Formula Vb is amino and R⁵ in Formula Vb is cyano. In some such embodiments, R^{3a} is C₁₋₆ alkyl or C₁₋₆ haloalkyl in Formula Vb. In some such embodiments, R^{3a} is C₁₋₆ alkyl or C₁₋₆ haloalkyl in Formula Vb and R⁴ is hydrogen or C₁₋₆ alkyl.

In some embodiments, the present disclosure provides a compound according to Formula I which is selected from any combination of the compounds provided in Table 1 below, or a pharmaceutically acceptable salt thereof.

TABLE 1
Compounds of the present disclosure

| Compound | | Compound | |
|---|---|---|---|
| No. | Structure | No. | Structure |
| 1 | 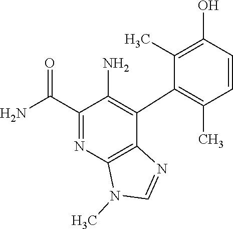 [SMILES: Cc1ccc(O)c(C)c1-c1c(N)c(C(N)=O)nc2c1ncn2C] | 2 | 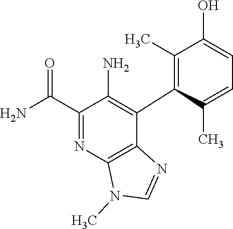 |
| 3 | 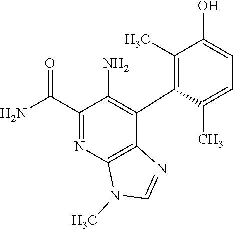 | 4 | 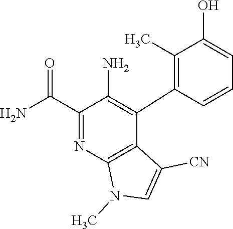 [SMILES: Cc1c(O)cccc1-c1c(N)c(C(N)=O)nc2c1c(C#N)cn2C] |
| 5 | 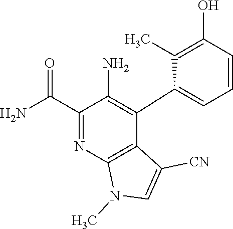 | 6 | 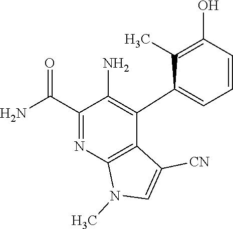 |
| 7 | 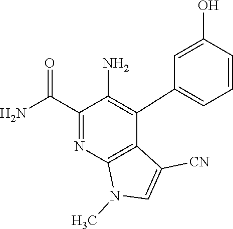 [SMILES: Cn1cc(C#N)c2c(-c3cccc(O)c3)c(N)c(C(N)=O)nc21] | 8 | 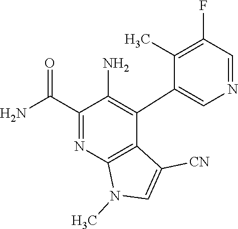 [SMILES: Cc1c(F)cncc1-c1c(N)c(C(N)=O)nc2c1c(C#N)cn2C] |
| 9 | 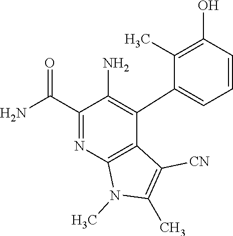 [SMILES: Cc1c(O)cccc1-c1c(N)c(C(N)=O)nc2c1c(C#N)c(C)n2C] | 10 | 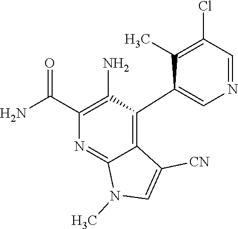 |
| 11 | 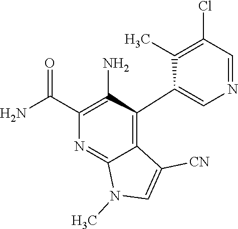 | 12 | 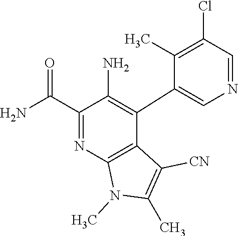 [SMILES: Cc1c(Cl)cncc1-c1c(N)c(C(N)=O)nc2c1c(C#N)c(C)n2C] |
| 13 | 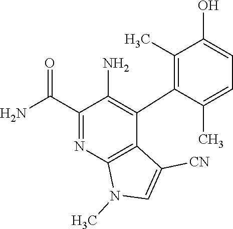 [SMILES: Cc1ccc(O)c(C)c1-c1c(N)c(C(N)=O)nc2c1c(C#N)cn2C] | 14 | 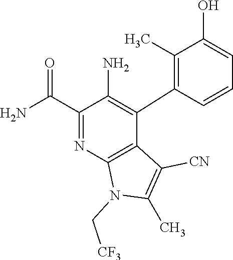 [SMILES: Cc1c(O)cccc1-c1c(N)c(C(N)=O)nc2c1c(C#N)c(C)n2CC(F)(F)F] |
| 15 | 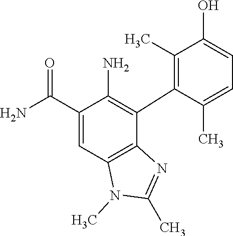 [SMILES: Cc1ccc(O)c(C)c1-c1c(N)c(C(N)=O)cc2c1nc(C)n2C] | 16 | 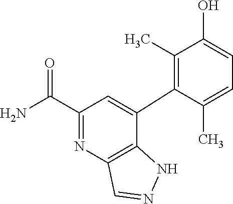 [SMILES: Cc1ccc(O)c(C)c1-c1cc(C(N)=O)nc2cn[nH]c12] |
| 17 | 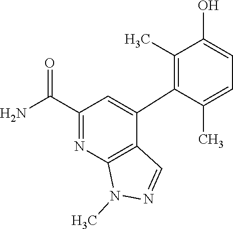 [SMILES: Cc1ccc(O)c(C)c1-c1cc(C(N)=O)nc2c1cnn2C] | 18 | 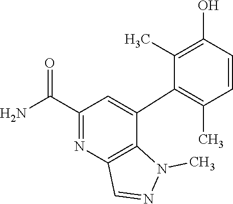 [SMILES: Cc1ccc(O)c(C)c1-c1cc(C(N)=O)nc2cnn(C)c12] |
| 19 | 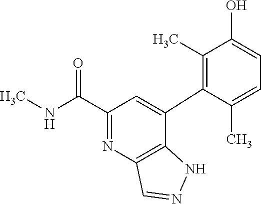 [SMILES: CNC(=O)c1cc(-c2c(C)ccc(O)c2C)c2[nH]ncc2n1] | 20 | 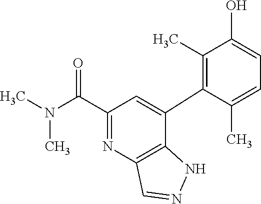 [SMILES: Cc1ccc(O)c(C)c1-c1cc(C(=O)N(C)C)nc2cn[nH]c12] |
| 21 | 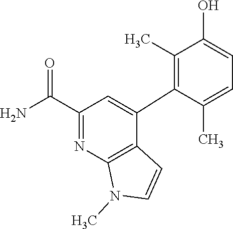 [SMILES: Cc1ccc(O)c(C)c1-c1cc(C(N)=O)nc2c1ccn2C] | 22 | 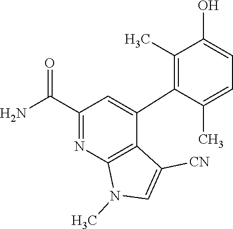 [SMILES: Cc1ccc(O)c(C)c1-c1cc(C(N)=O)nc2c1c(C#N)cn2C] |
| 23 | 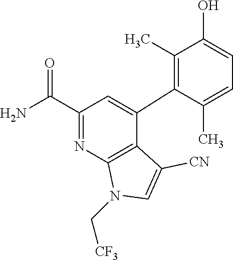 [SMILES: Cc1ccc(O)c(C)c1-c1cc(C(N)=O)nc2c1c(C#N)cn2CC(F)(F)F] | 24 | 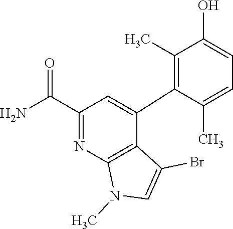 [SMILES: Cc1ccc(O)c(C)c1-c1cc(C(N)=O)nc2c1c(Br)cn2C] |
| 25 | 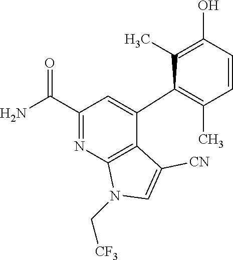 | 26 | 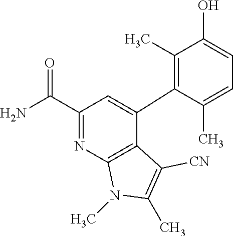 [SMILES: Cc1ccc(O)c(C)c1-c1cc(C(N)=O)nc2c1c(C#N)c(C)n2C] |
| 27 | 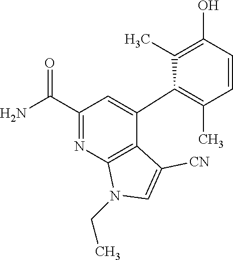 | 28 | 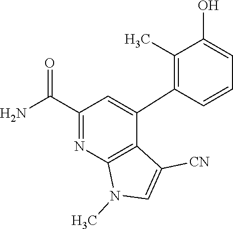 [SMILES: Cc1c(O)cccc1-c1cc(C(N)=O)nc2c1c(C#N)cn2C] |
| 29 | 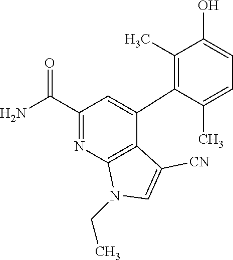 [SMILES: CCn1cc(C#N)c2c(-c3c(C)ccc(O)c3C)cc(C(N)=O)nc21] | 30 | 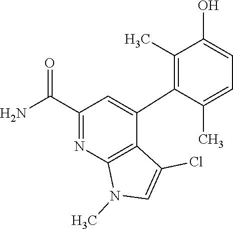 [SMILES: Cc1ccc(O)c(C)c1-c1cc(C(N)=O)nc2c1c(Cl)cn2C] |
| 31 | 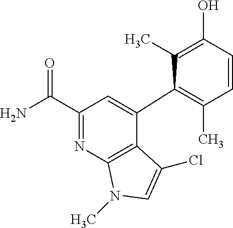 | 32 | 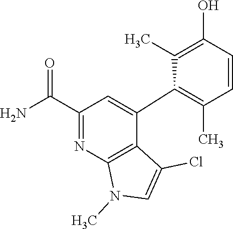 |
| 33 | 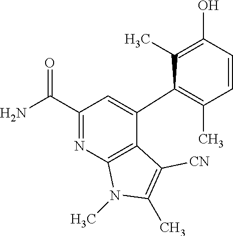 | 34 | 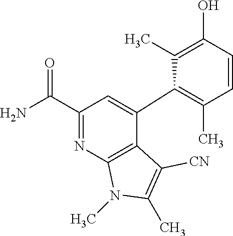 |
| 35 | 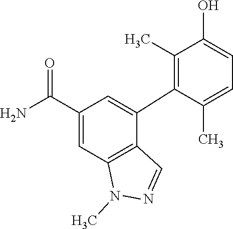 [SMILES: Cc1ccc(O)c(C)c1-c1cc(C(N)=O)cc2c1cnn2C] | 36 | 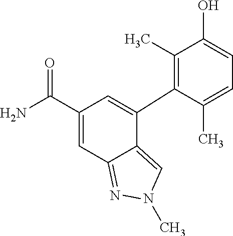 [SMILES: Cc1ccc(O)c(C)c1-c1cc(C(N)=O)cc2nn(C)cc12] |
| 37 | 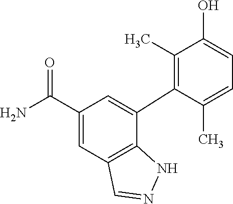 [SMILES: Cc1ccc(O)c(C)c1-c1cc(C(N)=O)cc2cn[nH]c12] | 38 | 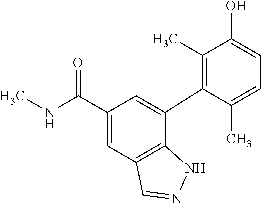 [SMILES: CNC(=O)c1cc(-c2c(C)ccc(O)c2C)c2[nH]ncc2c1] |
| 39 | 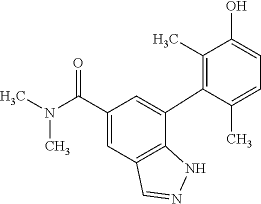 [SMILES: Cc1ccc(O)c(C)c1-c1cc(C(=O)N(C)C)cc2cn[nH]c12] | 40 | 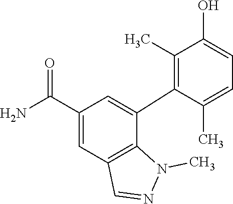 [SMILES: Cc1ccc(O)c(C)c1-c1cc(C(N)=O)cc2cnn(C)c12] |
| 41 | 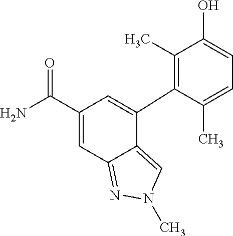 [SMILES: Cc1ccc(O)c(C)c1-c1cc(C(N)=O)cc2nn(C)cc12] | 42 | 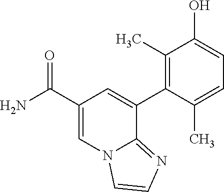 [SMILES: Cc1ccc(O)c(C)c1-c1cc(C(N)=O)cn2ccnc12] |
| 43 | 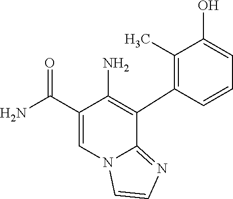 [SMILES: Cc1c(O)cccc1-c1c(N)c(C(N)=O)cn2ccnc12] | 44 | 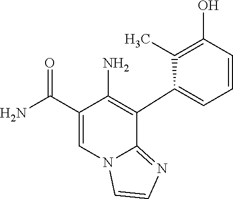 |
| 45 | 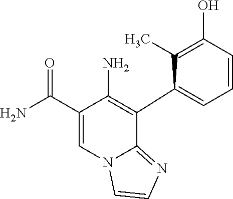 | 46 | 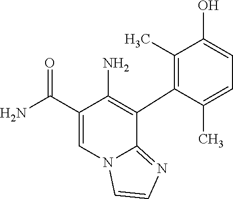 [SMILES: Cc1ccc(O)c(C)c1-c1c(N)c(C(N)=O)cn2ccnc12] |
| 47 | 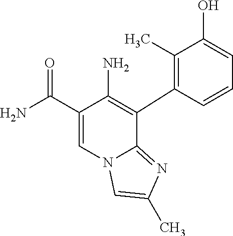 [SMILES: Cc1cn2cc(C(N)=O)c(N)c(-c3cccc(O)c3C)c2n1] | 48 | 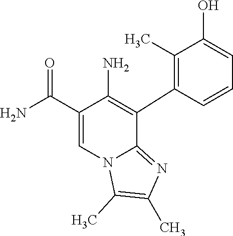 [SMILES: Cc1nc2c(-c3cccc(O)c3C)c(N)c(C(N)=O)cn2c1C] |
| 49 | 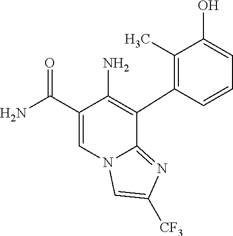 [SMILES: Cc1c(O)cccc1-c1c(N)c(C(N)=O)cn2cc(C(F)(F)F)nc12] | 50 | 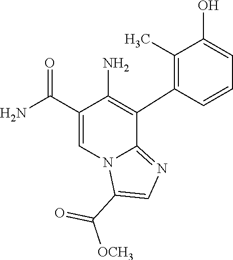 [SMILES: COC(=O)c1cnc2c(-c3cccc(O)c3C)c(N)c(C(N)=O)cn12] |
| 51 | 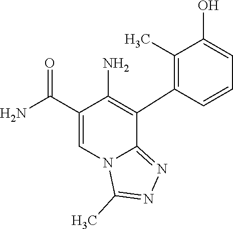 [SMILES: Cc1c(O)cccc1-c1c(N)c(C(N)=O)cn2c(C)nnc12] | 52 | 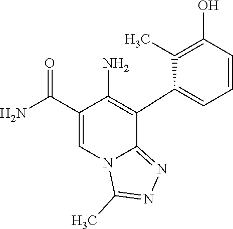 |
| 53 | 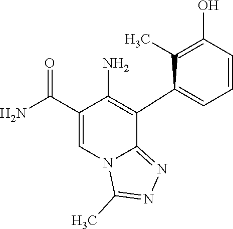 | 54 | 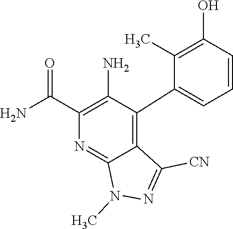 [SMILES: Cc1c(O)cccc1-c1c(N)c(C(N)=O)nc2c1c(C#N)nn2C] |
| 55 | 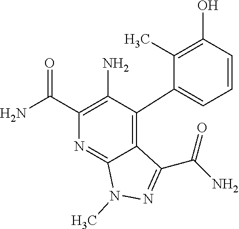 [SMILES: Cc1c(O)cccc1-c1c(N)c(C(N)=O)nc2c1c(C(N)=O)nn2C] | 56 | 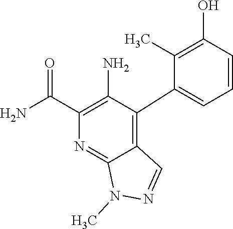 [SMILES: Cc1c(O)cccc1-c1c(N)c(C(N)=O)nc2c1cnn2C] |
| 57 | 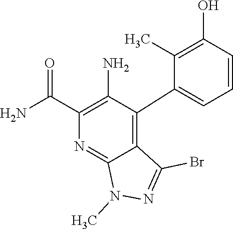 [SMILES: Cc1c(O)cccc1-c1c(N)c(C(N)=O)nc2c1c(Br)nn2C] | 58 | 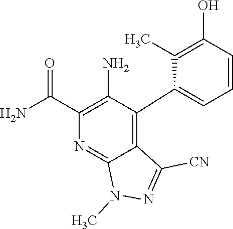 |
| 59 | 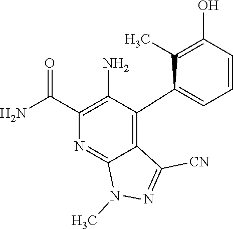 | 60 | 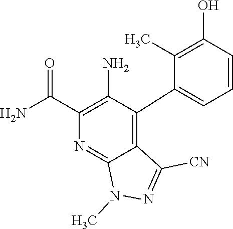 [SMILES: Cc1c(O)cccc1-c1c(N)c(C(N)=O)nc2c1c(C#N)nn2C] |
| 61 | 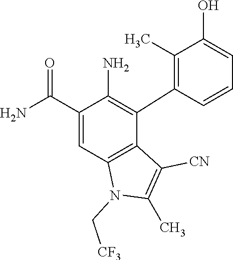 [SMILES: Cc1c(O)cccc1-c1c(N)c(C(N)=O)cc2c1c(C#N)c(C)n2CC(F)(F)F] | 62 | 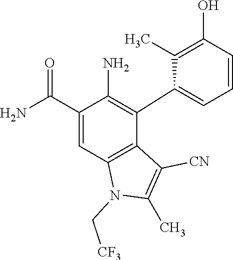 |
| 63 | 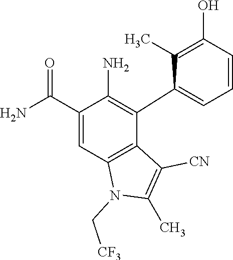 | 64 | 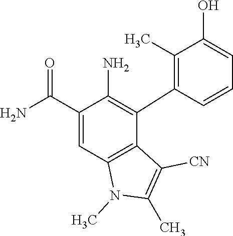 [SMILES: Cc1c(O)cccc1-c1c(N)c(C(N)=O)cc2c1c(C#N)c(C)n2C] |
| 65 | 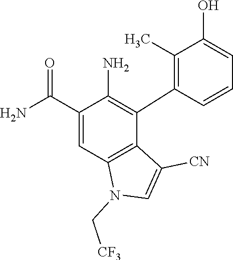 [SMILES: Cc1c(O)cccc1-c1c(N)c(C(N)=O)cc2c1c(C#N)cn2CC(F)(F)F] | 66 | 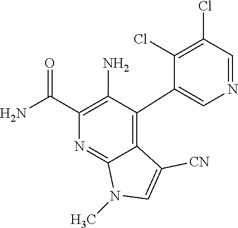 [SMILES: Cn1cc(C#N)c2c(-c3cncc(Cl)c3Cl)c(N)c(C(N)=O)nc21] |
| 67 | 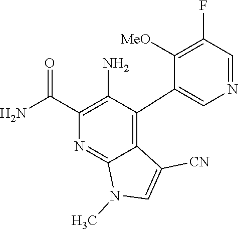 [SMILES: COc1c(F)cncc1-c1c(N)c(C(N)=O)nc2c1c(C#N)cn2C] | 68 | 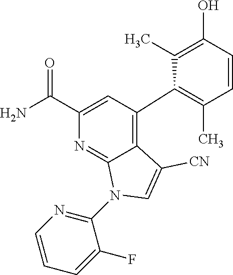 |
| 69 | 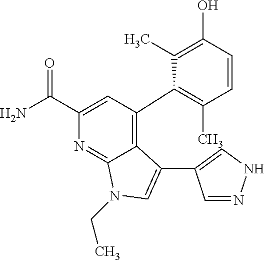 | 70 | 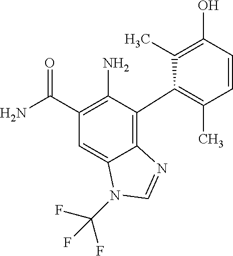 |
| 71 | 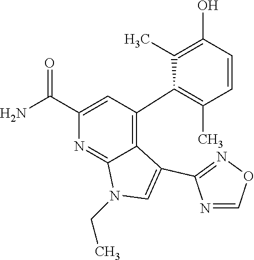 | 72 | 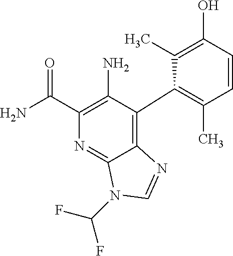 |
| 73 | 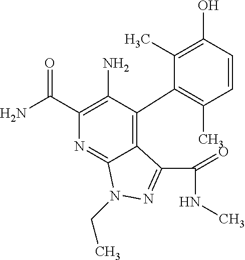 [SMILES: CCn1nc(C(=O)NC)c2c(-c3c(C)ccc(O)c3C)c(N)c(C(N)=O)nc21] | 74 | 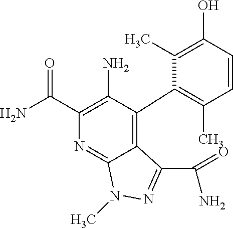 |
| 75 | 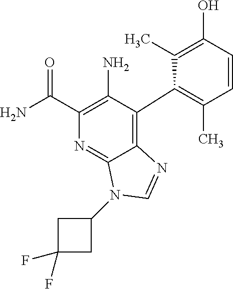 | 76 | 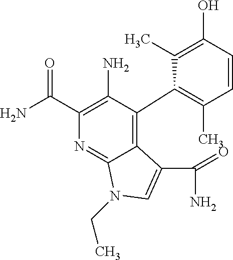 |
| 77 | 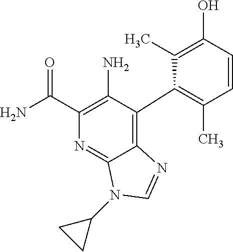 | 78 | 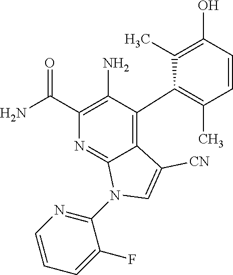 |
| 79 | 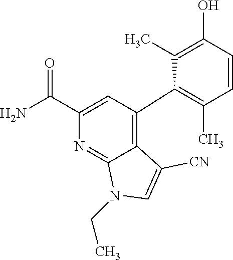 | 80 | 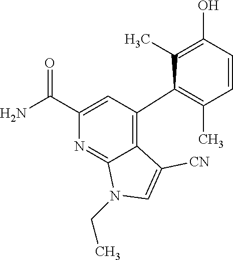 |
| 81 | 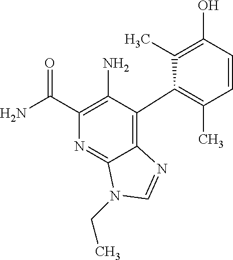 | 82 | 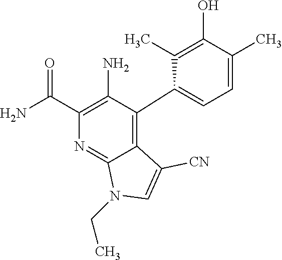 |
| 83 | 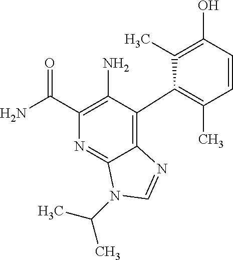 | 84 | 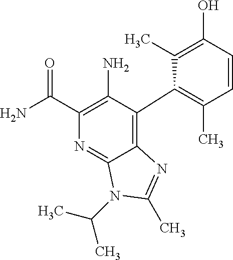 |
| 85 | 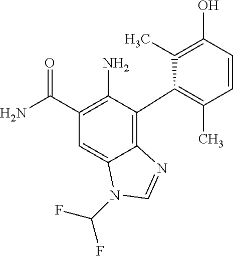 | 86 | 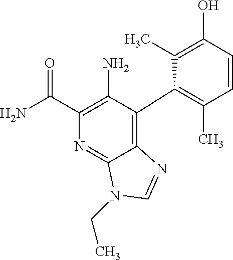 |
| 87 | 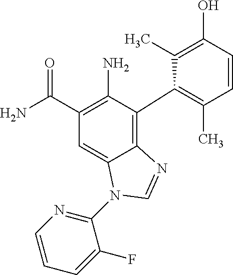 | 88 | 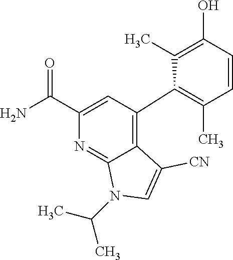 |
| 89 | 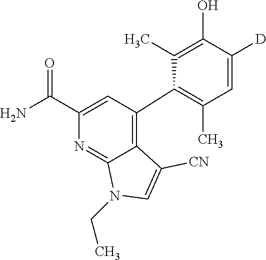 | 90 | 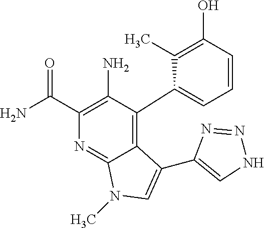 |
| 91 | 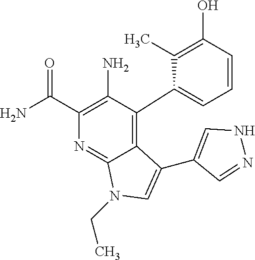 | 92 | 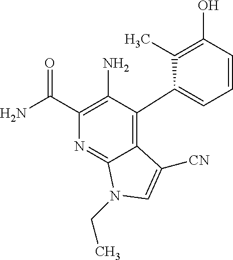 |
| 93 | 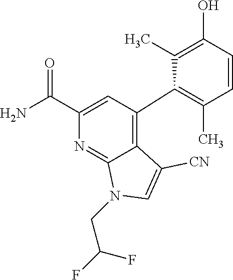 | 94 | 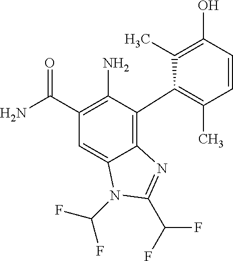 |
| 95 | 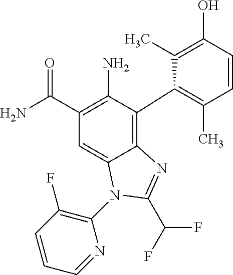 | 96 | 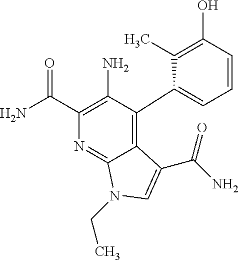 |
| 97 | 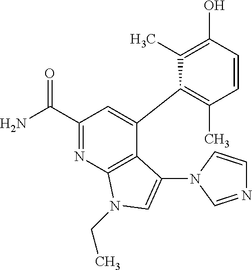 | 98 | 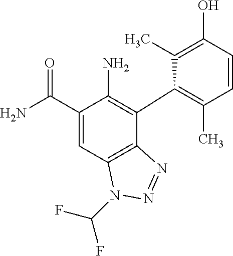 |

### Pharmaceutical Composition and Medical Treatment or Uses

Another aspect provides a pharmaceutical composition comprising any of the compounds of Formula I disclosed herein, or a pharmaceutically acceptable salt thereof, and one or more pharmaceutically acceptable excipients.

Compounds of the present disclosure inhibit PKMYT1 and are thus useful in the treatment or prevention of a variety of diseases and conditions. In particular, compounds of the present disclosure are useful in methods of treating or preventing a disease or condition wherein inhibition of PKMYT1 provides a benefit. It has further been discovered that particular compounds exhibit advantageously low rates of hepatic metabolic clearance as assessed, for example, by stability assays employing liver microsomes or hepatocytes obtained from humans or other species. Certain compounds of the present disclosure have also exhibited an advantageously high selectivity for inhibition of PKMYT1 over non-target kinases, such as Src-family kinases Lck and Lyn that regulate signal transduction. Accordingly, another aspect provides methods for treating cancer comprising administering a therapeutically effective amount of a compound of Formula I disclosed herein, or a pharmaceutically acceptable salt thereof, or a pharmaceutical composition comprising any of the compounds of Formula I disclosed herein, or a pharmaceutically acceptable salt thereof, to a subject in need thereof. Examples of cancers for treatment according to the methods include, but are not limited to, cancers of the adrenal glands, bladder, brain and spine, breast, cervix, colorectal, endometrium, esophagus, head & neck, blood and lymph, kidneys, liver, lung, ovaries, pancreas, skin, soft tissue (sarcoma), stomach, and uterus. In some embodiments, the cancer is a cancer overexpressing CCNE1. In some embodiments, the cancer is a cancer expressing one or more mutations in F-box/WD repeat-containing protein 7 (FBXW7) and/or protein phosphatase 2 regulatory subunit A alpha (PPP2RIA). A related aspect provides for the use of any of the compounds of Formula I disclosed herein, or a pharmaceutically acceptable salt thereof, or use of a pharmaceutical composition comprising any of the compounds of Formula I disclosed herein, or a pharmaceutically acceptable salt thereof in methods for treating cancer, or in the manufacture of medicaments for treating cancer. In some embodiments, the cancer in such uses is a cancer overexpressing CCNE1 or expressing one or more mutations in FBXW7 and/or PPP2RIA.

Administration of the compounds provided herein, or their pharmaceutically acceptable salts, in pure form or in an appropriate pharmaceutical composition, can be carried out via any of the accepted modes of administration or agents for serving similar utilities. Thus, administration can be, for example, oral, nasal, parenteral (intravenous, intramuscular, or subcutaneous), topical, transdermal, intravaginal, intravesical, intracisternal, or rectal, in the form of solid, semi-solid, lyophilized powder, or liquid dosage forms, such as, for example, tablets, suppositories, pills, soft elastic and hard gelatin capsules, powders, solutions, suspensions, aerosols, and the like, optionally in unit dosage forms suitable for simple administration of precise dosages.

The compositions will typically include a conventional pharmaceutical carrier or excipient and a compound of Formula I as the/an active agent, and, in addition, carriers, adjuvants, and the like. Excipients may include, for example, preserving, wetting, suspending, sweetening, flavoring, perfuming, emulsifying, and dispensing agents. Prevention of the action of microorganisms can be ensured by various antibacterial and antifungal agents. If desired, a pharmaceutical composition may also contain substances such as wetting or emulsifying agents, pH buffering agents, isotonic agents, and antioxidants.

Compositions suitable for parenteral injection may comprise physiologically acceptable sterile aqueous or nonaqueous solutions, dispersions, suspensions or emulsions, and sterile powders for reconstitution into sterile injectable solutions or dispersions.

Solid dosage forms for oral administration include capsules, tablets, pills, powders, and granules.

Liquid dosage forms for oral administration include pharmaceutically acceptable emulsions, solutions, suspensions, syrups, and elixirs. Such dosage forms are prepared, for example, by dissolving, dispersing, or otherwise combining, a compound(s) of Formula I, or a pharmaceutically acceptable salt thereof, and optional pharmaceutical adjuvants in an aqueous or non-aqueous carrier to thereby form a solution or suspension. Suspensions, in addition to the active compounds, may contain suspending agents.

Compositions for rectal administration include, for example, suppositories that can be prepared by mixing the compounds of the present invention with suitable non-irritating excipients or carriers which are solid at ordinary temperatures but liquid at body temperature and therefore melt while in a suitable body cavity and release the active component therein.

Dosage forms for topical administration include ointments, powders, sprays, and inhalants. The active component is typically admixed under sterile conditions with a physiologically acceptable carrier and any preservatives, buffers, or propellants as may be required. Ophthalmic formulations, eye ointments, powders, and solutions are also contemplated for use in topical administration.

Generally, depending on the intended mode of administration, the pharmaceutically acceptable compositions will contain about 1% to about 99% by weight of a compound(s) of the invention, or a pharmaceutically acceptable salt thereof, and 99% to 10% by weight of a suitable pharmaceutical excipient. In one example, the composition will be between about 5% and about 75% by weight of a compound(s) of the invention, or a pharmaceutically acceptable salt thereof, with the rest being suitable pharmaceutical excipients. Methods of preparing such dosage forms are known, or will be apparent, to those skilled in this art; for example, see Remington The Science and Practice of Pharmacy, 23^{rd} Ed., (Academic Press 2020). The composition to be administered will, in any event, contain a therapeutically effective amount of a compound as described herein, or a pharmaceutically acceptable salt thereof, for treatment of a disease-state as described herein.

The compounds described herein (e.g., compounds of Formula I), or their pharmaceutically acceptable salts, are generally administered in a therapeutically effective amount which will vary depending upon a variety of factors including the activity of the specific compound employed, the metabolic stability and length of action of the compound, the age, body weight, general health, sex, diet, mode, and time of administration, rate of excretion, drug combination, the severity of the particular disease-states, and the host undergoing therapy. The compounds of the present invention can be administered, for example, to a patient at dosage levels in the range of about 0.1 to about 1,000 mg per day.

### General Synthetic Methods

Compounds of this invention can be made by the synthetic procedures described below. These schemes are merely illustrative of some methods by which the compounds of this invention can be synthesized, and various modifications to these schemes can be made. The starting materials and the intermediates of the reaction may be isolated and purified if desired using conventional techniques, including but not limited to filtration, distillation, crystallization, chromatography, and the like. Such materials may be characterized using conventional means, including physical constants and spectral data.

The compounds disclosed and claimed herein have asymmetric carbon atoms or quaternized nitrogen atoms in their structure and may be prepared through the syntheses described herein as single stereoisomers, racemates, or mixtures of enantiomers and diastereomers. The compounds may also exist as geometric isomers. All such single stereoisomers, racemates, and geometric isomers, and mixtures thereof are intended to be within the scope of this invention.

The compounds may also exist as atropisomers, which are conformational stereoisomers that result from hindered rotation about a single bond where the steric strain barrier to rotation can be high enough to allow for the isolation of each conformer. The compounds provided herein include all atropisomers, both as pure individual atropisomer preparations, enriched preparations of each, or a non-specific mixture of each.

Some of the compounds of the invention may exist as tautomers. For example, where a ketone or aldehyde is present, the molecule may exist in the enol form; where an amide is present, the molecule may exist as the imidic acid; and where an enamine is present, the molecule may exist as an imine. All such tautomers are within the scope of the invention.

Various methods for the preparation and/or separation and isolation of single stereoisomers from racemic mixtures or non-racemic mixtures of stereoisomers. For example, supercritical fluid chromatography (SFC) and/or HPLC can be used for separation of chiral molecules. Further, optically active isomers may be prepared using chiral synthons or chiral reagents or resolved using conventional techniques. Enantiomers (R- and S-isomers) and atropisomers (M- and P-isomers) may be resolved, for example, by: formation of diastereomeric salts or complexes which may be separated, for example, by crystallization; via formation of diastereomeric derivatives which may be separated, for example, by crystallization; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent.

In addition, the compounds can exist in unsolvated as well as solvated forms with pharmaceutically acceptable solvents such as water, ethanol, and the like. In general, the solvated forms are considered equivalent to the unsolvated forms for the purposes of the present invention.

Processes for preparation of compounds according to the present disclosure may be carried out as semi-continuous or continuous processes, more preferably as continuous processes. Reactions in the processes may be carried out in the presence of a solvent or a mixture of two or more solvents, or the reactions may be carried out in the absence of additional solvents. In particular, the solvent may be an aqueous solvent or an organic solvent such as an ether (e.g., tetrahydrofuran, methyltetrahydrofuran, diisopropyl ether, t-butyl methyl ether, or dibutyl ether), aliphatic hydrocarbon solvent (e.g., hexane, heptane, or pentane), saturated alicyclic hydrocarbon solvent (e.g., cyclohexane or cyclopentane), or aromatic solvent (e.g., toluene, o-, m-, or p-xylene, or t-butyl-benzene), or mixture thereof. The starting materials and reagents, which do not have their synthetic route explicitly disclosed herein, are generally available from commercial sources or are readily prepared using known methods.

The compounds disclosed and claimed herein can be prepared according to the general Scheme A, as well as Schemes 1-30 below.

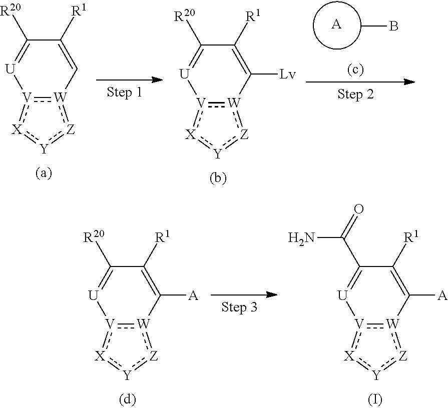

In the general scheme, variables such as ring A, U, V, W, X, Y, Z, R¹, and R^{a} are as defined herein. R²⁰ is —COOR^{a}, —CONH₂, or CN. “Lv” refers to a leaving group including, but not limited to halogen, mesylates, tosylates and the like.

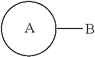

is a boronic acid or boronic ester of ring A.

One aspect of the present disclosure provides a process for making a compound of Formula I,

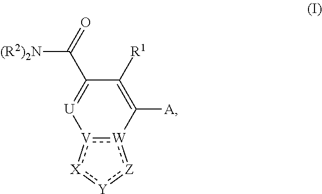

or a pharmaceutically acceptable salt thereof, comprising
(a) combining a compound of Formula (b) and a compound of Formula (c) to form a compound of Formula (d)

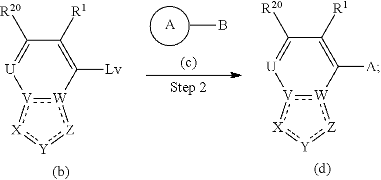

and
(b) converting the compound of Formula (d) to yield the compound of Formula I,

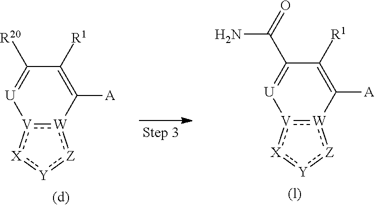

wherein
Lv is a leaving group;
the compound of Formula (c) is a boronic acid or boronic ester of ring A;
each [symbol] represents a single bond, a double bond, or a delocalized π bond;
U is selected from the group consisting of N and CH;
V and W are independently selected from the group consisting of N and C;
X is selected from the group consisting of CR³, N. NR^{3a}, S, and O;
Y is selected from the group consisting of CR⁴, N. NR^{4a}, S, and O;
Z is selected from the group consisting of CR⁵, N, NR^{5a}, S, and O;
ring A is
3- to 14-membered carbocyclyl substituted with 1, 2, 3, 4, or 5 R⁶, or
5- to 14-membered heterocyclyl comprising at least one oxygen atom, nitrogen atom, or sulfur atom, wherein the heterocyclyl is substituted with 0, 1, 2, 3, or 4 R⁶;
R¹ is selected from the group consisting of amino, hydroxy, and hydrogen;
each R² is independently selected from the group consisting of hydrogen and C₁₋₆ alkyl;
R³, R⁴, and R⁵ are independently selected from the group consisting of hydrogen, amino, hydroxy, halogen, cyano, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl. —NO₂, —CO(C₁₋₆ alkyl), —COOH, —COO(C₁₋₆ alkyl), —CON(R⁷)₂, -L-(5- to 12-membered heterocyclyl), -L-(5- to 14-membered heteroaryl, and -L-(C₆₋₁₄ aryl), wherein the C₃₋₈ cycloalkyl, 5- to 12-membered heterocyclyl, 5- to 14-membered heteroaryl, and C₆₋₁₄ aryl are independently substituted with 0, 1, 2, 3, or 4 R⁶;
each L is independently selected from the group consisting of a covalent bond, —O—, C₁₋₆ alkylene, and NR^{a};
any two adjacent R³, R⁴, and R⁵ are optionally taken together with the carbon atoms to which they are attached to form a 5- or 6-membered carbocyle or heterocycle, each of which is optionally substituted with 0, 1, 2, 3, or 4 R⁶;
R^{3a}, R^{4a}, R^{5a}, and R⁷ are independently selected from the group consisting of hydrogen. C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, -L-(5- to 12-membered heterocyclyl), -L-(5- to 14-membered heteroaryl, and -L-(C₆₋₁₄ aryl), wherein the 5- to 12-membered heterocyclyl, 5- to 14-membered heteroaryl, and C₆₋₁₄ aryl are independently substituted with 0, 1, 2, 3, or 4 R⁶;
each R⁶ is independently selected from the group consisting of hydroxy, amino, cyano, halogen, C₁₋₆ alkyl, C₁₋₆ haloalkyl, C₁₋₆ hydroxyalkyl, C₁₋₆ alkoxy, oxo, —NO₂, —CHO, —CO(C₁₋₆ alkyl), —COOR^{a}, —CON(R^{a})₂, —SO₂(C₁₋₆ alkyl), and —SO₂N(R^{a})₂; and
R²⁰ is —COOR^{a}, —COONH₂, or CN; and
each R^{a} is independently selected from the group consisting of hydrogen. C₁₋₆ alkyl, and C₁₋₆ haloalkyl.

In some embodiments, the process further comprises converting a compound of Formula (a) to the compound of Formula (b):

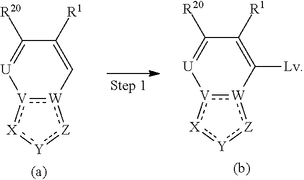

In some embodiments, the compound of Formula I is a compound of Formula Ia, Ib, Ic, Id, IIa, IIb, IIc, IId, IIIa, or IIIb, as described and defined herein.

In some embodiments, the compound of Formula (c) is:

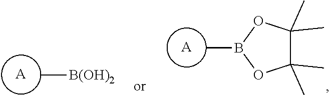

wherein ring A is as defined herein.

In some embodiments, Lv is halogen. In some embodiments, Lv is —Br. In some embodiments, Lv is —Cl.

In step 1 of the process, when Lv is halogen, the compound of Formula (b) can be prepared by halogenation of the compound of Formula (a). The reaction can be carried out by reacting the compound of Formula (a) with chlorine, bromine, NBS, or the like in the presence of a suitable solvent. Examples of suitable solvents include, but are not limited to, DMF, THF, DMSO, acetonitrile, ether, ketone, and 1,4-dioxane.

Step 2 of the reaction can be carried out in the presence of a suitable base, catalyst, and solvent. The base may be an inorganic base or an organic base. Non-limiting examples of inorganic bases include bicarbonates, carbonates, phosphates, and acetates. Examples of organic bases include, but are not limited to, acyclic amines, e.g., tertiary amines, pyridine, and piperidine. The catalyst may be any catalyst suitable for Suzuki-type couplings, and includes but is not limited to a nickel, palladium, or platinum catalyst. Non-limiting examples of such catalysts include NiCl₂(dppf), SPhosPdG₃, Pd(PPh₃)₄, PdCl₂(PPh₃)₂, Pd(dppf)Cl₂-DCM, and Pd₂(dba)₃. Suitable solvents include, but are not limited to, protic and aprotic solvents such as water, methanol, ethanol, butanol, toluene, DMF, DME, DCM, THF, DMSO, ether, ketone, 1,4-dioxane, and the like. Combinations of two, three, or more solvents may also be employed. The reaction can be carried out in a temperature ranging from room temperature (about 20° C.) to about 200° C. In some embodiments, the reaction temperature is about 80° C. to about 150° C. In some embodiments, the reaction temperature is about 100° C. to about 120° C.

In step 3 of the process, the compound of Formula (d) can be converted to the compound of Formula I by amidation or partial hydrolysis. When R²⁰ is —COOR^{a}, the compound of Formula (d) can react with NH₃ in the presence of an alcohol such as methanol, ethanol, butanol, or the like. When R²⁰ is —CN, the nitrile can be hydrolyzed to a carboxamide by acid or base hydrolysis. The acid hydrolysis can be carried out in the presence of a strong mineral acid, such as sulfuric acid, and water. The base hydrolysis can be carried out in the presence of a strong base such as NaOH, LiOH, or KOH. Hydrogen peroxide can also be used for the base hydrolysis.

In some embodiments, the compound of Formula I exists as atropisomers. The atropisomers can be separated by chiral SFC.

The following examples are provided for the purpose of further illustration and are not intended to limit the scope of the claimed invention.

### Synthetic Examples

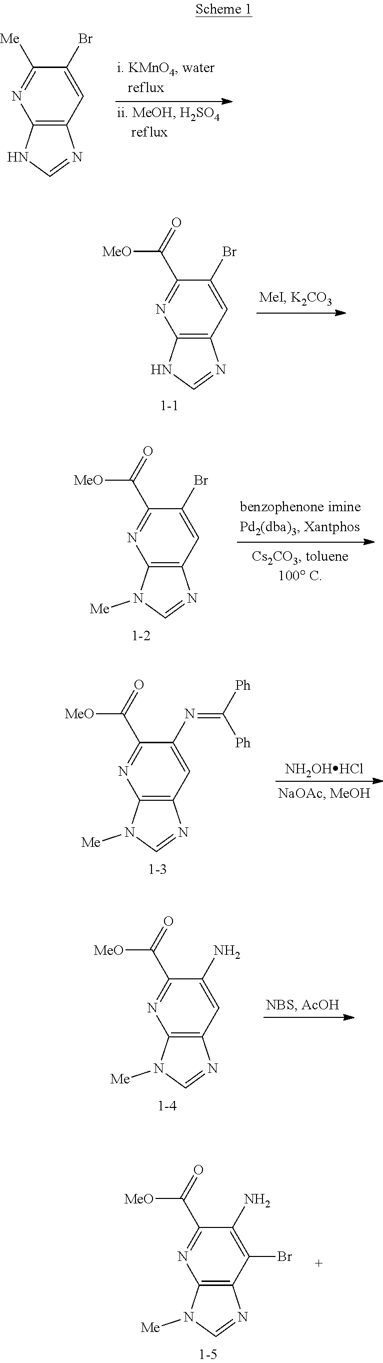

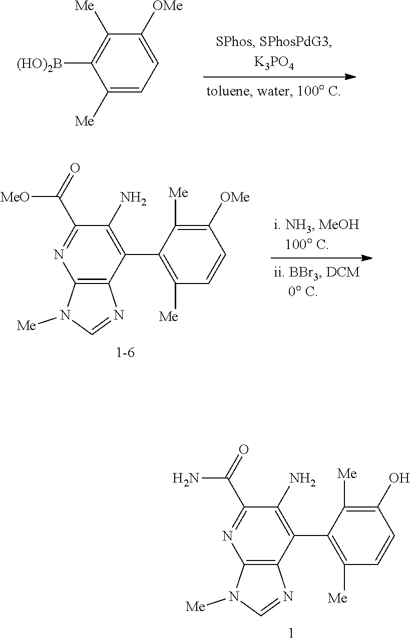 [SMILES: COc1ccc(C)c(B(O)O)c1C ; COC(=O)c1nc2c(ncn2C)c(-c2c(C)ccc(OC)c2C)c1N ; Cc1ccc(O)c(C)c1-c1c(N)c(C(N)=O)nc2c1ncn2C]

### Example 1. 6-Amino-7-(3-hydroxy-2,6-dimethylphenyl)-3-methyl-3H-imidazo[4,5-b]pyridine-5-carboxamide (1)

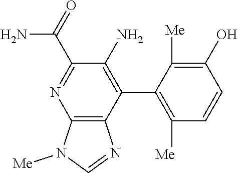 [SMILES: Cc1ccc(O)c(C)c1-c1c(N)c(C(N)=O)nc2c1ncn2C]

### Step 1: Synthesis of methyl 6-bromo-3H-imidazo[4,5-b]pyridine-5-carboxylate

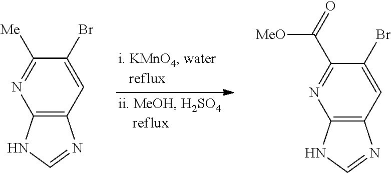 [SMILES: Cc1nc2[nH]cnc2cc1Br ; COC(=O)c1nc2[nH]cnc2cc1Br]

6-Bromo-5-methyl-3H-imidazo[4,5-b]pyridine (2.0 g, 9.43 mmol, 1.00 equiv.) was suspended in water (30 mL), then KMnO₄ (2.0 g, 13 mmol, 1.3 equiv.) was added. The reaction was heated to reflux for 1 hour. More KMnO₄ (2.0 g, 13 mmol, 1.3 equiv.) was added, and the reaction was heated at reflux for 2 hours. More KMnO₄ was added (3.0 g, 19 mmol, 2.0 equiv.), and the reaction heated at reflux for 1.5 hours. The reaction was hot filtered through Celite® and washed with hot water. The filtrate was concentrated and used directly in the next step without further purification.

The crude residue was suspended in MeOH (110 mL), and concentrated sulfuric acid (H₂SO₄, 5.0 mL, 93 mmol, 9.9 equiv.) was added. The reaction was heated to reflux for 3 hours. The reaction was basified with a saturated aqueous solution of Na₂CO₃ until pH>11. The reaction mixture was concentrated in vacuo until most of the MeOH was removed. Then EtOAc (100 mL) and water (100 mL) were added, and the layers were separated. The organic layer was collected; the aqueous layer was reextracted with EtOAc (75 mL). The organic layers were combined, dried with Na₂SO₄, filtered, and concentrated to afford the title compound (710 mg, 29% yield). LC-MS m/z 256 (M+H⁺).

### Step 2: Synthesis of methyl 6-bromo-3-methyl-3H-imidazo[4,5-b]pyridine-5-carboxylate

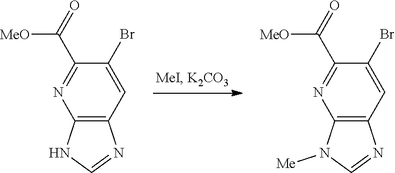 [SMILES: COC(=O)c1nc2[nH]cnc2cc1Br ; COC(=O)c1nc2c(cc1Br)ncn2C]

Methyl 6-bromo-3H-imidazo[4,5-b]pyridine-5-carboxylate (710 mg, 2.77 mmol, 1.00 equiv.) was dissolved in DMF (6.0 mL) under an atmosphere of nitrogen. K₂CO₃ (766 mg, 5.54 mmol, 2.00 equiv.) and MeI (0.345 mL, 5.54 mmol, 2.00 equiv.) were added, and the reaction was stirred at room temperature for 2 hours. The reaction was diluted with DCM (50 mL) and water (50 mL), and the layers were separated. The organic layer was collected, and the aqueous layer was reextracted 2× with DCM (50 mL). The organic layers were combined, dried with Na₂SO₄, filtered, concentrated onto Celite®, and subjected to FCC (MeOH:DCM 0:100 to 5:95) to afford the title compound (415 mg, 55% yield) as a single isomer. NMR spectroscopy was used to confirm the regioselectivity. ¹H NMR (400 MHz, CDCl₃) δ 8.34-8.28 (m, 1H), 8.14 (d. J=2.4 Hz, 1H), 4.05-3.98 (m, 3H), 3.92 (d, J=2.5 Hz, 3H). LC-MS m/z 270 (M+H⁺).

### Step 3: Synthesis of methyl 6-((diphenylmethylene)amino)-3-methyl-3H-imidazo[4,5-b]pyridine-5-carboxylate

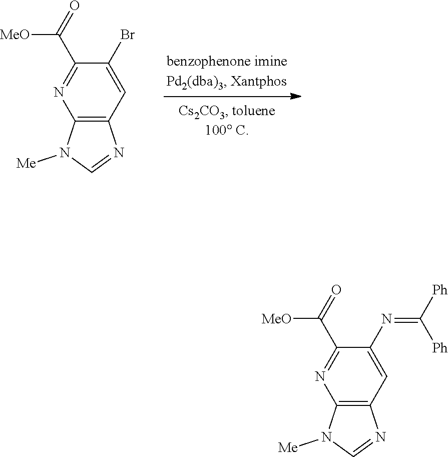 [SMILES: COC(=O)c1nc2c(cc1Br)ncn2C ; COC(=O)c1nc2c(cc1N=C(c1ccccc1)c1ccccc1)ncn2C]

Methyl 6-bromo-3-methyl-3H-imidazo[4,5-b]pyridine-5-carboxylate (600 mg, 2.22 mmol, 1.00 equiv.), tris(dibenzylideneacetone)dipalladium(0) (Pd₂(dba)₃. 161 mg, 0.176 mmol, 0.079 equiv.), 4,5-Bis(diphenylphosphino)-9,9-dimethylxanthene (Xantphos, 200 mg, 0.356 mmol, 0.156 equiv.), and Cs₂CO₃ (1.1 g, 3.4 mmol, 1.5 equiv.) were added to a microwave tube under an atmosphere of nitrogen. Toluene (11 mL) was added, and the reaction was sparged with nitrogen for 2 mins. Benzophenone imine (0.50 mL, 2.98 mmol, 1.34 equiv.) was added, and the reaction was sparged for a further 5 mins. The reaction was heated in the microwave at 100° C. for 12 hours. The reaction was diluted with EtOAc (150 mL) and filtered through Celite®. The solvent was removed, and the residue was adsorbed onto Celite® and subjected to FCC (MeOH:DCM 0:100 to 10:90) to afford the title compound (650 mg, 79% yield). LC-MS m/z 371 (M+H⁺).

### Step 4: Synthesis of methyl 6-amino-3-methyl-3H-imidazo[4,5-b]pyridine-5-carboxylate

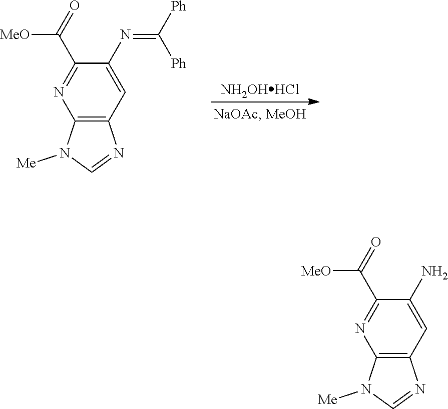

Methyl 6-((diphenylmethylene)amino)-3-methyl-3H-imidazo[4,5-b]pyridine-5-carboxylate (600 mg, 1.62 mmol, 1.0 equiv.) was dissolved in MeOH (17 mL). Hydroxylamine hydrochloride (NH₂OH HCl, 157 mg, 2.26 mmol, 1.40 equiv.) and sodium acetate (NaOAc, 2.26 mmol, 1.70 equiv) were added, and the reaction was stirred at room temperature for 2 hours. The reaction was quenched with 0.1 M NaOH in water (30 mL) and diluted with DCM (50 mL). The layers were separated. The aqueous layer was reextracted 2× with DCM (50 mL). The organic layers were collected, dried with Na₂SO₄, filtered, and concentrated. The residue was adsorbed onto silica and subjected to FCC (MeOH:DCM 0:100 to 15:85) to afford the title compound (260 mg, 78% yield). LC-MS m/z 207 (M+H⁺).

### Step 5: Synthesis of methyl 6-amino-7-bromo-3-methyl-3H-imidazo[4,5-b]pyridine-5-carboxylate

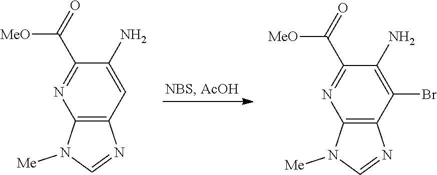 [SMILES: COC(=O)c1nc2c(cc1N)ncn2C ; COC(=O)c1nc2c(ncn2C)c(Br)c1N]

Methyl 6-amino-3-methyl-3H-imidazo[4,5-b]pyridine-5-carboxylate (250 mg, 1.21 mmol, 1.00 equiv.) was dissolved in AcOH (2.5 mL) under an atmosphere of nitrogen. NBS (240 mg, 1.34 mmol, 1.11 equiv.) was added, and the reaction was stirred at room temperature for 1.5 hours. EtOAc (100 mL) was added followed by a saturated aqueous solution of Na₂CO₃ (50 mL), and the layers were separated. The aqueous layer was reextracted 2× with EtOAc. The combined organic layers were dried with Na₂SO₄, filtered, and concentrated. The residue was adsorbed onto Celite® and subjected to FCC (MeOH:DCM 0:100 to 15:85) to afford the title compound (220 mg, 64% yield). LC-MS m/z 285 (M+H⁺).

### Step 6: Synthesis of methyl 6-amino-7-(3-methoxy-2,6-dimethylphenyl)-3-methyl-3H-imidazo[4,5-b]pyridine-5-carboxylate

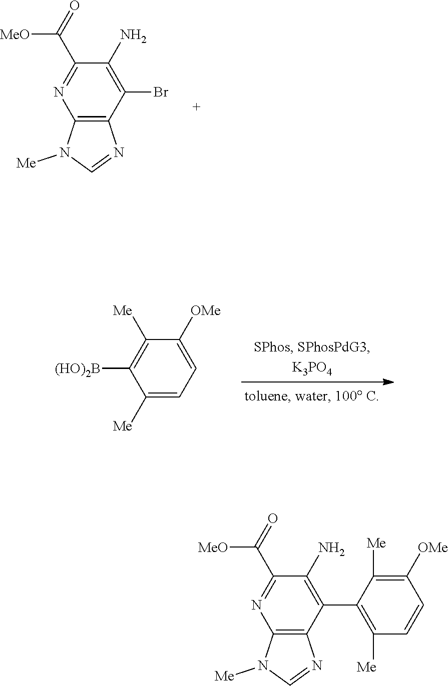 [SMILES: COC(=O)c1nc2c(ncn2C)c(Br)c1N ; COc1ccc(C)c(B(O)O)c1C ; COC(=O)c1nc2c(ncn2C)c(-c2c(C)ccc(OC)c2C)c1N]

Methyl 6-amino-7-bromo-3-methyl-3H-imidazo[4,5-b]pyridine-5-carboxylate (130 mg, 0.829 mmol, 1.00 equiv.), (3-methoxy-2,6-dimethylphenyl)boronic acid (164 mg, 0.911 mmol, 2.00 equiv.), dicyclohexyl(2′,6′-dimethoxy[1,1′-biphenyl]-2-yl)phosphane (SPhos, 37 mg, 0.090 mmol, 0.20 equiv.). K₃PO₄ (193 mg, 0.909 mmol, 2.00 equiv.) was suspended in toluene (2.6 mL) and water (0.3 mL). The reaction was degassed for 2 mins, then (2-dicyclohexylphosphino-2′,6′-dimethoxybiphenyl) [2-(2′-amino-1,1′-biphenyl)]palladium(II) methanesulfonate (SPhos Pd G3, 71 mg, 0.091 mmol, 0.20 equiv.) was added. The reaction was degassed for another 5 minutes. The reaction was heated in the microwave to 100° C. for 6 hours. The reaction was diluted with DCM (50 mL) and water (40 mL), and the layers were separated. The organic layer was collected, and the aqueous layer was reextracted 3× with DCM. The organic layers were combined, dried with Na₂SO₄, filtered, and concentrated. The residue was adsorbed onto silica and then subjected to FCC (MeOH:DCM 0:100 to 10:90) to afford the title compound (170 mg, 95% yield) as red foam. LC-MS m/z 341 (M+H⁺).

### Step 7: Synthesis of 6-amino-7-(3-hydroxy-2,6-dimethylphenyl)-3-methyl-3H-imidazo[4,5-b]pyridine-5-carboxamide (Compound 1)

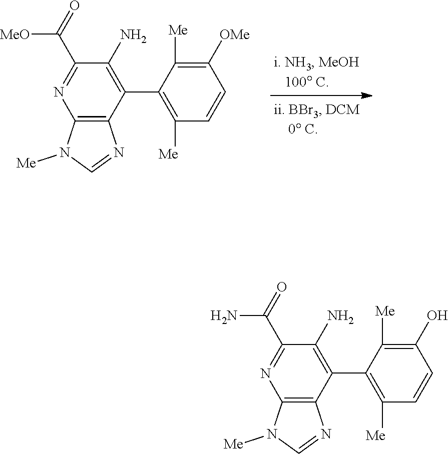 [SMILES: COC(=O)c1nc2c(ncn2C)c(-c2c(C)ccc(OC)c2C)c1N ; Cc1ccc(O)c(C)c1-c1c(N)c(C(N)=O)nc2c1ncn2C]

Methyl 6-amino-7-(3-methoxy-2,6-dimethylphenyl)-3-methyl-3H-imidazo[4,5-b]pyridine-5-carboxylate (165 mg, 0.485 mmol, 1.00 equiv.) was dissolved in a solution of ammonia in MeOH (7M, 12 mL, 77 mmol, 159 equiv.) in a microwave tube. The reaction was heated to 110° C. for 15 hours. The solvent was removed, and the crude residue was used in the next step without further purification.

The crude residue was dissolved in DCM (10 mL) under an atmosphere of nitrogen gas and cooled to 0° C. A solution of BBr₃ in DCM (1 M, 2.5 mL, 2.5 mmol, 3.00 equiv.) was added dropwise over 1 min and the mixture was stirred at 0° C. for 2 hours. Methanol (4 mL) was added, and the solvent was removed. The residue was dissolved in a 1:1 mixture of water and DMF, filtered, and subjected to RP-HPLC (0.1% FA, Water:MeCN, 95:5 to 0:100) to afford the title compound (30 mg, 19% yield). ¹H NMR (400 MHz, DMSO) δ 9.18 (s, 1H), 8.24-8.18 (m, 1H), 8.13 (s, 1H), 7.42 (s, 1H), 6.91 (d, J=8.1 Hz, 1H), 6.74 (d, J=8.0 Hz, 1H), 5.74 (s, 2H), 3.75 (d, J=2.6 Hz, 3H), 1.68 (d, J=2.5 Hz, 3H), 1.62 (d, J=2.5 Hz, 3H). LC-MS m/z 312 (M+H⁺).

### Example 2. (P)-6-Amino-7-(3-hydroxy-2,6-dimethylphenyl)-3-methyl-3H-imidazo[4,5-b]pyridine-5-carboxamide (2) and (M)-6-amino-7-(3-hydroxy-2,6-dimethylphenyl)-3-methyl-3H-imidazo[4,5-b]pyridine-5-carboxamide (3)

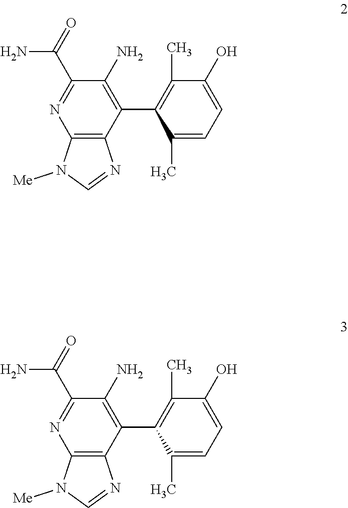

Chiral SFC separation of compound 1 (50 mg, 0.17 mmol) (Instrument: Waters SFC Prep 150 Mgm; Column: Daicel Chiralpak OJ, 30×150 mm, 5 um; Conditions: Isocratic at 20% methanol with 80% CO₂: Flow Rate: 100 mL/min) provided compound 2 (15 mg, 30% yield) and compound 3 (15 mg, 30% yield).

Peak 1: SFC t_{R}=1.60 min. LC-MS m/z 312 [M+H]⁺; ¹H NMR (400 MHz, DMSO) δ 9.22 (s, 1H), 8.28 (d, J=2.9 Hz, 1H), 8.20 (s, 1H), 7.49 (s, 1H), 6.99 (d, J=7.5 Hz, 1H), 6.87-6.69 (m, 1H), 5.82 (s, 2H), 3.82 (t, J=2.1 Hz, 3H), 1.72 (dd, J=25.6, 3.0 Hz, 6H).

Peak 2: SFC t_{R}=2.42 min. LC-MS m/z 312 [M+H]⁺; ¹H NMR (400 MHz, DMSO) δ 9.22 (t, J=1.8 Hz, 1H), 8.31-8.26 (m, 1H), 8.20 (s, 1H), 7.49 (s, 1H), 6.99 (d, J=8.2 Hz, 1H), 6.84-6.78 (m, 1H), 5.82 (s, 2H), 3.82 (t, J=1.9 Hz, 3H), 1.78-1.67 (m, 6H).

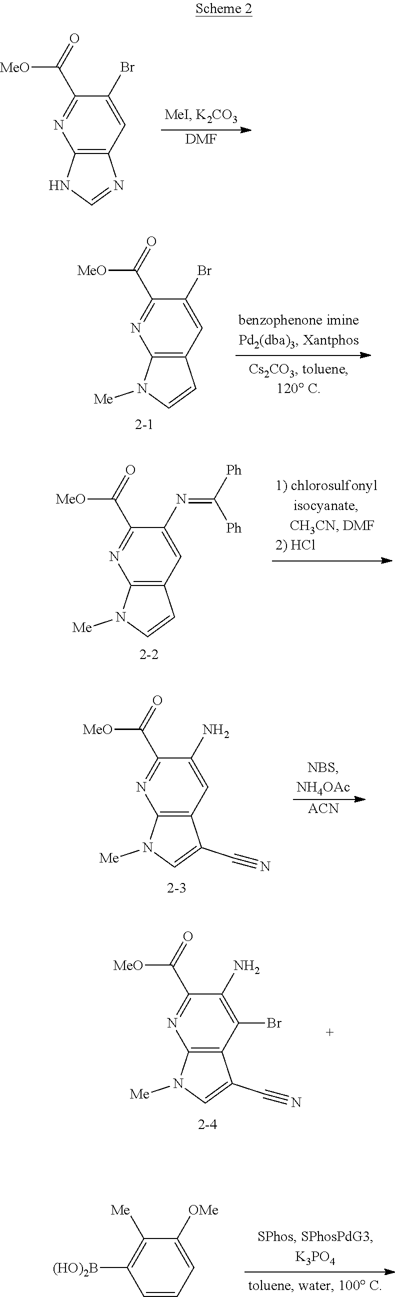

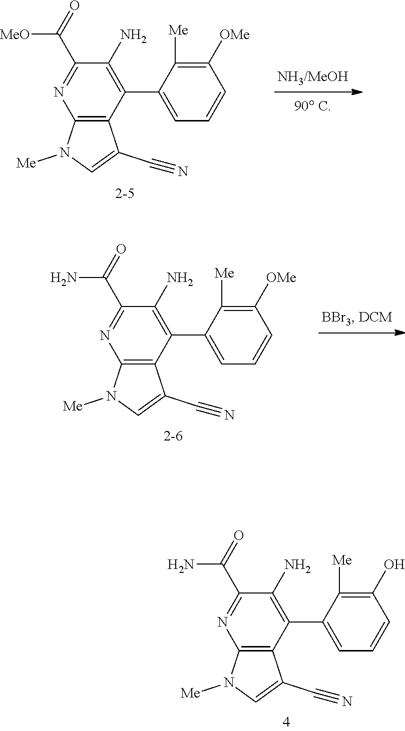 [SMILES: COC(=O)c1nc2c(c(C#N)cn2C)c(-c2cccc(OC)c2C)c1N ; COc1cccc(-c2c(N)c(C(N)=O)nc3c2c(C#N)cn3C)c1C ; Cc1c(O)cccc1-c1c(N)c(C(N)=O)nc2c1c(C#N)cn2C]

### Example 3. 5-Amino-3-cyano-4-(3-hydroxy-2-methylphenyl)-1-methyl-1H-pyrrolo[2,3-b]pyridine-6-carboxamide (4)

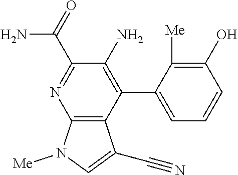 [SMILES: Cc1c(O)cccc1-c1c(N)c(C(N)=O)nc2c1c(C#N)cn2C]

### Step 1: Synthesis of methyl 5-bromo-1-methyl-1H-pyrrolo[2,3-b]pyridine-6-carboxylate

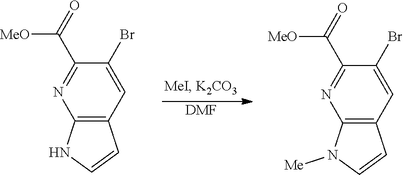

Methyl 5-bromo-1H-pyrrolo[2,3-b]pyridine-6-carboxylate (1 g, 3.92 mmol, 1.00 equiv.) was dissolved in DMF (6.0 mL) under an atmosphere of nitrogen. K₂CO₃ (1100 mg, 7.96 mmol, 2.03 equiv.) and MeI (0.5 mL, 8 mmol, 2.05 equiv.) were added, and the reaction was stirred at room temperature for 2.5 hours. Additional K₂CO₃ (360 mg, 2.60 mmol, 0.66 equiv.) and MeI (0.2 mL, 3.21 mmol, 0.82 equiv) were added, and the reaction mixture was stirred for another 1.5 hours. The reaction was diluted with EtOAc (50 mL), and water (50 mL) and the layers were separated. The organic layer was collected, and the aqueous layer was re-extracted with EtOAc (50 mL). The organic layers were combined, washed with water and brine, and dried with MgSO₄. The solution was filtered and concentrated to afford the title compound (1060 mg, 100% yield). LC-MS m/z 269 (M+H⁺).

### Step 2: Synthesis of methyl 5-((diphenylmethylene)amino)-1-methyl-1H-pyrrolo[2,3-b]pyridine-6-carboxylate

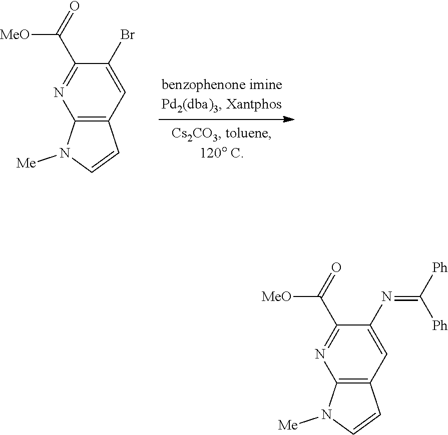 [SMILES: COC(=O)c1nc2c(ccn2C)cc1Br ; COC(=O)c1nc2c(ccn2C)cc1N=C(c1ccccc1)c1ccccc1]

A pressure tube was charged with methyl 5-bromo-1-methyl-1H-pyrrolo[2,3-b]pyridine-6-carboxylate (3.21 g, 11.9 mmol, 1.00 equiv.), tris(dibenzylideneacetone)dipalladium(0) (Pd₂(dba)₃, 1.64 g, 1.79 mmol, 0.15 equiv.), 4,5-bis(diphenylphosphino)-9,9-dimethylxanthene (Xantphos, 2.08 g, 3.59 mmol, 0.30 equiv.), and Cs₂CO₃ (7.77 g, 23.8 mmol, 2.0 equiv.). The tube was evacuated and refilled with nitrogen. Degassed toluene (70 mL) and benzophenone imine (3 mL, 17.9 mmol, 1.50 equiv.) were added, and the reaction was degassed for 5 min. The mixture was heated in an oil bath at 120° C. for 12 hours. The reaction was diluted with EtOAc and filtered through Celite®. The solvent was removed, and residue was adsorbed onto silica and subjected to FCC (EtOAc:hexane 5:95 to 60:40) to afford the title compound (3.51 g, 80% yield). LC-MS m/z 370 (M+H⁺).

### Step 3: Synthesis of methyl 5-amino-3-cyano-1-methyl-1H-pyrrolo[2,3-b]pyridine-6-carboxylate

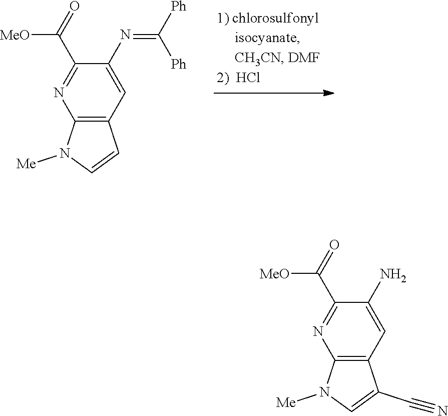 [SMILES: COC(=O)c1nc2c(ccn2C)cc1N=C(c1ccccc1)c1ccccc1 ; COC(=O)c1nc2c(cc1N)c(C#N)cn2C]

Methyl 5-((diphenylmethylene)amino)-1-methyl-1H-pyrrolo[2,3-b]pyridine-6-carboxylate (1.43 g, 3.87 mmol, 1.00 equiv) was dissolved in anhydrous DMF (15 mL) under an atmosphere of nitrogen and cooled to −20° C. A solution of chlorosulfonyl isocyanate (1.40 mL, 16.0 mmol, 4.20 equiv.) in anhydrous CH₃CN (12 mL) was added dropwise over 20 min. The mixture was warmed to 0° C. and stirred at this temperature for 50 min. 1M HCl (15 mL) was slowly added to quench the reaction, and the mixture was stirred at 0° C. for an additional 15 min. Saturated NaHCO₃ was added to adjust the pH to 8-9, and the resulting suspension was extracted with EtOAc until no more solid was present. The organic layers were combined, dried over MgSO₄, filtered, and concentrated. The residue was adsorbed onto silica and subjected to FCC (EtOAc:hexane 10:90 to 100:0) to afford the title compound (450 mg, 51% yield). LC-MS m/z 231 (M+H⁺).

### Step 4: Synthesis of methyl 5-amino-4-bromo-3-cyano-1-methyl-1H-pyrrolo[2,3-b]pyridine-6-carboxylate

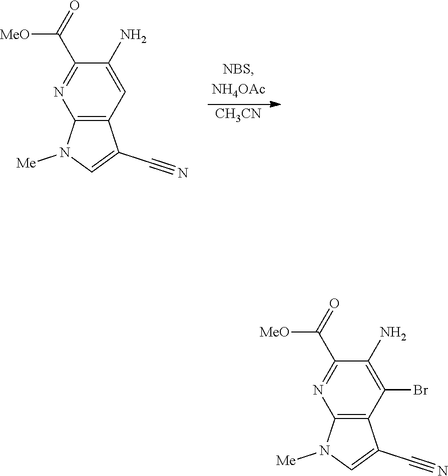 [SMILES: COC(=O)c1nc2c(cc1N)c(C#N)cn2C ; "CH3CN" ; COC(=O)c1nc2c(c(C#N)cn2C)c(Br)c1N]

Methyl 5-amino-3-cyano-1-methyl-1H-pyrrolo[2,3-b]pyridine-6-carboxylate (450 mg, 1.95 mmol, 1.00 equiv.) and ammonium acetate (20 mg, 0.26 mmol, 0.13 equiv.) were suspended in CH₃CN (13 mL) under an atmosphere of nitrogen. A solution of NBS (366 mg, 2.06 mmol, 1.05 equiv.) in CH₃CN (13 mL) was added, and the reaction was stirred at room temperature for 20 min. Solvent was evaporated, and the residue was adsorbed onto silica and subjected to FCC (EtOAc:DCM 0:100 to 60:40). Fractions containing the desired product were combined and washed with water to remove succinimide. The aqueous layer was extracted with DCM until all the solids were dissolved. The organic layer was dried over MgSO₄, filtered, and concentrated to afford the title compound (417 mg, 69% yield). LC-MS m/z 309 (M+H⁺).

### Step 5: Synthesis of methyl 5-amino-3-cyano-4-(3-methoxy-2-methylphenyl)-1-methyl-1H-pyrrolo[2,3-b]pyridine-6-carboxylate

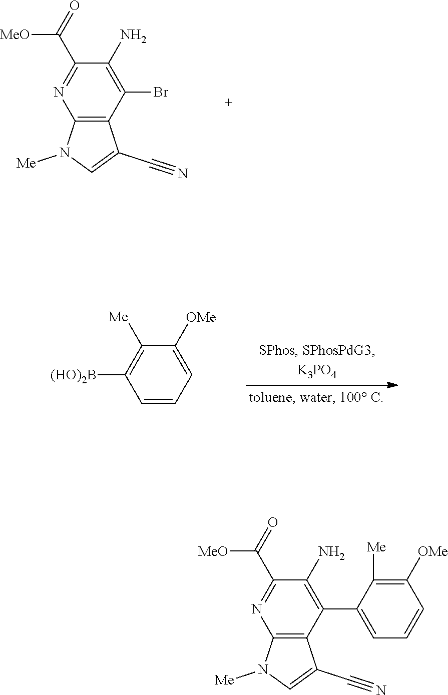 [SMILES: COC(=O)c1nc2c(c(C#N)cn2C)c(Br)c1N ; COc1cccc(B(O)O)c1C ; COC(=O)c1nc2c(c(C#N)cn2C)c(-c2cccc(OC)c2C)c1N]

A microwave vial was charged with methyl 5-amino-4-bromo-3-cyano-1-methyl-1H-pyrrolo[2,3-b]pyridine-6-carboxylate (480 mg, 1.55 mmol, 1.00 equiv.), (3-methoxy-2-methylphenyl)boronic acid (515 mg, 3.10 mmol, 2.00 equiv.), dicyclohexyl(2′,6′-dimethoxy[1,1′-biphenyl]-2-yl)phosphane (SPhos, 128 mg, 0.311 mmol, 0.20 equiv.), (2-dicyclohexylphosphino-2′,6′-dimethoxybiphenyl) [2-(2′-amino-1,1′-biphenyl)]palladium(II) methanesulfonate (SPhos Pd G3, 242 mg, 0.31 mmol, 0.20 equiv.) and K₃PO₄ (660 mg, 3.11 mmol, 2.00 equiv.). The vial was evacuated and refilled with nitrogen. Degassed toluene (3.8 mL) and water (0.42 mL) were added, and the suspension was degassed for another 5 min and sealed. The reaction was heated in the microwave to 100° C. for 1 hour. The resulting suspension was diluted with EtOAc and the solid was separated by decantation. The solid was dissolved in water and extracted with EtOAc. All the EtOAc washes were combined and filtered through Celite®. The filtrate was concentrated, and the residue was adsorbed onto silica and subjected to FCC (EtOAc:hexane 5:95 to 100:0). Additional product was obtained by subjecting the Celite® filter cake to FCC (eluting with the same gradient) to give the title compound (498 mg, 92% yield). LC-MS m/z 351 (M+H⁺).

### Step 6: Synthesis of 5-amino-3-cyano-4-(3-methoxy-2-methylphenyl)-1-methyl-1H-pyrrolo[2,3-b]pyridine-6-carboxamide

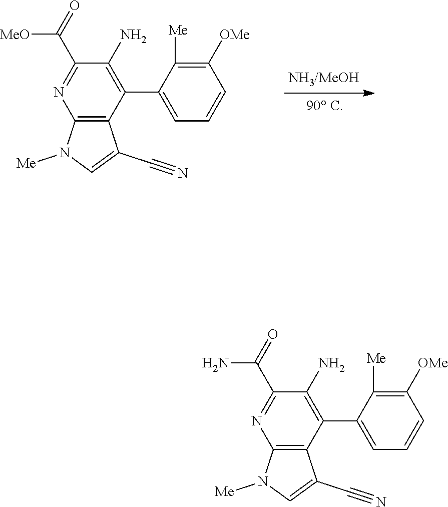 [SMILES: COC(=O)c1nc2c(c(C#N)cn2C)c(-c2cccc(OC)c2C)c1N ; COc1cccc(-c2c(N)c(C(N)=O)nc3c2c(C#N)cn3C)c1C]

Methyl 5-amino-3-cyano-4-(3-methoxy-2-methylphenyl)-1-methyl-1H-pyrrolo[2,3-b]pyridine-6-carboxylate (497 mg, 1.42 mmol, 1.00 equiv.) was suspended in a solution of ammonia in MeOH (7 M, 34 mL, 200 mmol, 200 equiv.). The mixture was divided into two microwave tubes and heated to 90° C. for 12 hours. The solvent was removed to give the title compound (480 mg, 100% yield). LC-MS m/z 336 (M+H⁺).

### Step 7: Synthesis of 5-amino-3-cyano-4-(3-hydroxy-2-methylphenyl)-1-methyl-1H-pyrrolo[2,3-b]pyridine-6-carboxamide (Compound 4)

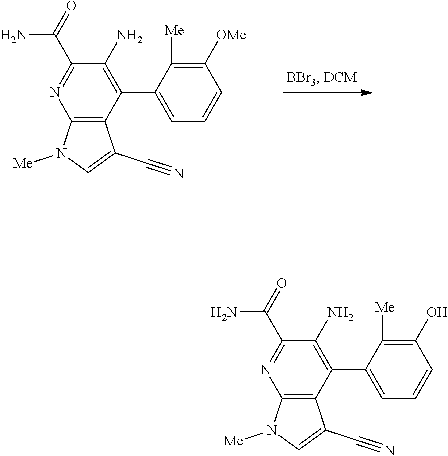 [SMILES: COc1cccc(-c2c(N)c(C(N)=O)nc3c2c(C#N)cn3C)c1C ; Cc1c(O)cccc1-c1c(N)c(C(N)=O)nc2c1c(C#N)cn2C]

A mixture of 5-amino-3-cyano-4-(3-methoxy-2-methylphenyl)-1-methyl-1H-pyrrolo[2,3-b]pyridine-6-carboxamide (400 mg, 1.19 mmol, 1.00 equiv) and anhydrous DCM (40 mL) was placed in an ice bath. A solution of BBr₃ in DCM (1 M, 6.0 mL, 6.0 mmol, 5.00 equiv.) was added dropwise, and the mixture was stirred at 0° C. for 1 hour, followed by at room temperature for 3 hours. The reaction mixture was re-cooled to 0° C. and slowly transferred to pre-cooled saturated NaHCO₃ (300 mL) at 0° C. The resulting mixture was extracted with EtOAc until no more fluorescent material was observed in the organic layer. The organic extracts were combined, dried over MgSO₄, and concentrated to give the crude product (410 mg, ~90% pure). This batch was subjected to SFC for chiral resolution directly. In a smaller scale synthesis, the crude solid was dissolved in DMF, filtered, and subjected to RP-HPLC (0.1% FA, Water:MeCN, 90:10 to 0:100) to afford the title compound (compound 4, 22 mg, 66% yield, 97% purity). 1H NMR (400 MHz, DMSO) δ 9.43 (s, 1H), 8.30 (s, 1H), 8.24 (s, 1H), 7.49 (s, 1H), 7.06 (t, J=8.0 Hz, 1H), 6.83 (d, J=7.5 Hz, 1H), 6.52 (d, J=6.9 Hz, 1H), 5.88 (s, 2H), 3.80 (s, 3H), 1.73 (s, 3H). LC-MS m/z 322 (M+H⁺).

### Example 4. (M)-5-Amino-3-cyano-4-(3-hydroxy-2-methylphenyl)-1-methyl-1H-pyrrolo[2,3-b]pyridine-6-carboxamide (5) and (P)-5-amino-3-cyano-4-(3-hydroxy-2-methylphenyl)-1-methyl-1H-pyrrolo[2,3-b]pyridine-6-carboxamide (6)

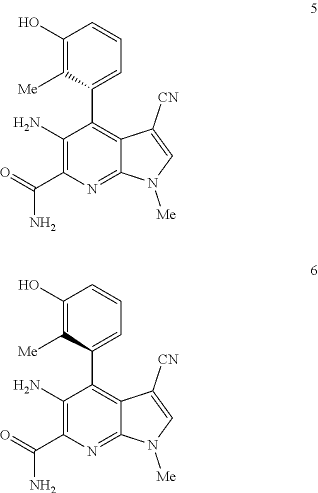

Compounds 5 and 6 were prepared through the chiral SFC separation of compound 4 (a racemate). Instrument: Waters SFC Prep 150 Mgm; Column: OJ, 30×250 mm, 5 um; Conditions: Isocratic at 30% methanol with 70% CO₂; Flow Rate: 100 mL/min) provided compound 5 (16.3 mg) and compound 6 (17.5 mg).

Peak 1: SFC t_{R}=1.79 min. ¹H NMR (400 MHz, d6-DMSO) δ 9.49 (s, 1H), 8.37 (s, 1H), 8.31 (s, 1H), 7.56 (s, 1H), 7.13 (t, J=7.4 Hz, 1H), 6.90 (d, J=8.4 Hz, 1H), 6.59 (d, J=7.6 Hz, 1H), 5.96 (s, 2H), 3.87 (s, 3H), 1.80 (s, 3H). LC-MS m/z 322 (M+H⁺).

Peak 2: SFC t_{R}=2.16 min. ¹H NMR (400 MHz, d6-DMSO) δ 9.49 (s, 1H), 8.37 (s, 1H), 8.31 (s, 1H), 7.56 (s, 1H), 7.13 (t, J=8.0 Hz, 1H), 6.90 (d, J=8.1 Hz, 1H), 6.59 (d, J=7.5 Hz, 1H), 5.96 (s, 2H), 3.87 (s, 3H), 1.80 (s, 3H). LC-MS m/z 322 (M+H⁺).

### Example 5. 5-Amino-3-cyano-4-(3-hydroxyphenyl)-1-methyl-1H-pyrrolo[2,3-b]pyridine-6-carboxamide (7)

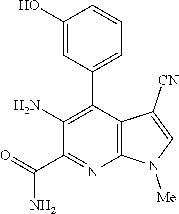 [SMILES: Cn1cc(C#N)c2c(-c3cccc(O)c3)c(N)c(C(N)=O)nc21]

Compound 7 was prepared by analogy to Example 3, using (3-methoxyphenyl)boronic acid in Step 5 (14.7 mg, 26% yield over 3 steps). ¹H NMR (400 MHz, DMSO) δ 9.62 (s, 1H), 8.40 (s, 1H), 8.32 (s, 1H), 7.56 (s, 1H), 7.34 (t, J=8.0 Hz, 1H), 6.86 (d, J=8.3 Hz, 1H), 6.76 (d, J=7.4 Hz, 1H), 6.73 (s, 1H), 6.12 (s, 2H), 3.87 (s, 3H). LC-MS m/z 308 (M+H⁺).

### Example 6. 5-Amino-3-cyano-4-(5-fluoro-4-methylpyridin-3-yl)-1-methyl-1H-pyrrolo[2,3-b]pyridine-6-carboxamide (8)

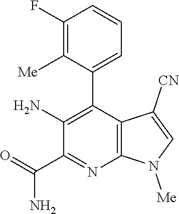 [SMILES: Cc1c(F)cccc1-c1c(N)c(C(N)=O)nc2c1c(C#N)cn2C]

Compound 8 was prepared by analogy to Example 3 using 3-fluoro-4-methyl-5-(4,4,5,5-tetramethyl-1,3,2-dioxaborolan-2-yl)pyridine in Step 5 to obtain the title compound (25.5 mg). ¹H NMR (400 MHz, DMSO) δ 12.77 (FAC, s, 0.43H), 8.61 (s, 1H), 8.44 (s, 1H), 8.38 (s, 1H), 8.24 (s, 1H), 8.14 (FAC, s, 0.43H), 7.62 (s, 1H), 6.42 (s, 2H), 3.89 (s, 3H), 1.99 (s, 3H). ¹⁹F NMR (376 MHz, DMSO) δ −132.07. LC-MS m/z 325 (M+H⁺).

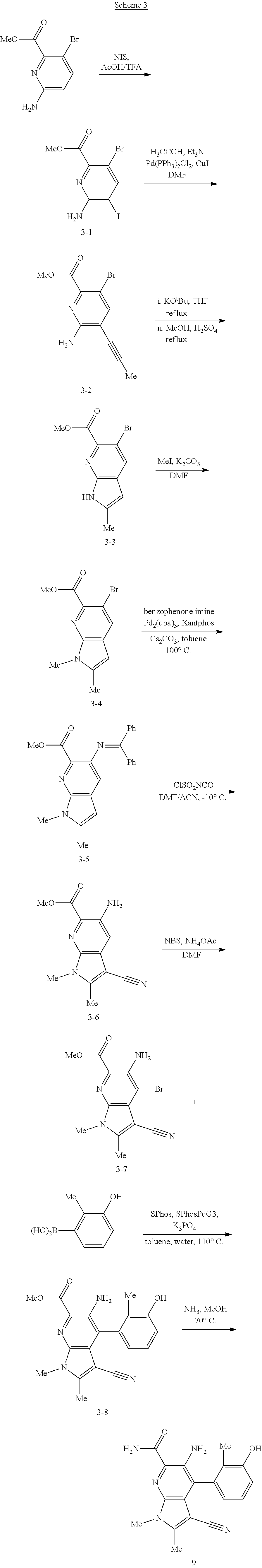

### Example 7. 5-Amino-3-cyano-4-(3-hydroxy-2-methylphenyl)-1,2-dimethyl-1H-pyrrolo[2,3-b]pyridine-6-carboxamide (9)

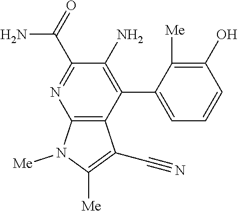 [SMILES: Cc1c(O)cccc1-c1c(N)c(C(N)=O)nc2c1c(C#N)c(C)n2C]

### Step 1: Synthesis of methyl 6-amino-3-bromo-5-iodopicolinate

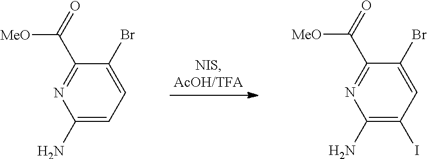 [SMILES: COC(=O)c1nc(N)ccc1Br ; COC(=O)c1nc(N)c(I)cc1Br]

Methyl 6-amino-3-bromopicolinate (16.74 g, 72.45 mmol, 1.00 equiv.) was suspended in acetic acid (AcOH, 167 mL) and trifluoroacetic acid (TFA, 4 mL) under an atmosphere of nitrogen. NIS (22.8 g, 101.4 mmol, 1.4 equiv.) was added, and the reaction was heated to 55° C. for 18 hours. The solvent was removed, and water (200 mL) was added. The suspension was stirred vigorously for 20 minutes and then filtered. The precipitate was collected and dried to afford the title compound (22.1 g, 85% yield). LC-MS m/z 357 (M+H⁺).

### Step 2: Synthesis of methyl 6-amino-3-bromo-5-(prop-1-yn-1-yl)picolinate

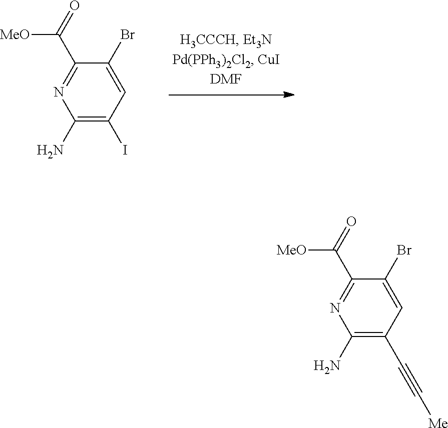 [SMILES: COC(=O)c1nc(N)c(I)cc1Br ; CC#Cc1cc(Br)c(C(=O)OC)nc1N]

Methyl 6-amino-3-bromo-5-iodopicolinate (3.72 g, 10.4 mmol, 1.00 equiv.), triethylamine (Et₃N, 7.0 mL, 53 mmol, 5.0 equiv.), and copper(I) iodide (CuI, 210 mg, 1.10 mmol, 0.106 equiv.) were suspended in a solution of propyne in DMF (1M, 15 mL, 15 mmol, 1.44 equiv.) under an atmosphere of nitrogen. Bis(triphenylphosphine)palladium(II) dichloride (Pd(PPh₃)₂Cl₂, 400 mg, 0.570 mmol, 0.0547 equiv.) was added, and the reaction was stirred at room temperature for 30 minutes. Water (100 mL) and EtOAc (100 mL) were added, and the layers were separated. The organic layer was collected, and washed with brine, dried with Na₂SO₄, filtered, and concentrated. The residue was adsorbed onto Celite® and subjected to FCC (EtOAc:hexanes 10:90 to 60:40) to afford the title compound (2.35 g, 84% yield). LC-MS m/z 269 (M+H⁺).

### Step 3: Synthesis of methyl 5-bromo-2-methyl-1H-pyrrolo[2,3-b]pyridine-6-carboxylate

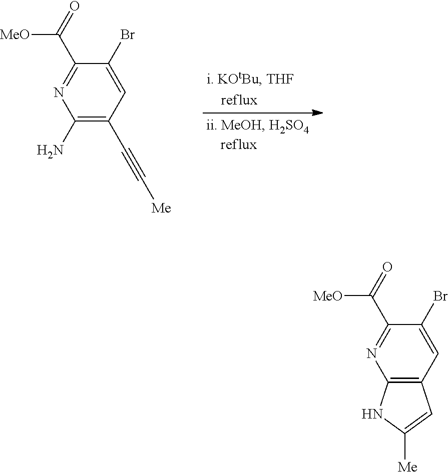 [SMILES: CC#Cc1cc(Br)c(C(=O)OC)nc1N ; COC(=O)c1nc2[nH]c(C)cc2cc1Br]

Methyl 6-amino-3-bromo-5-(prop-1-yn-1-yl)picolinate (2.34 g, 8.79 mmol, 1.00 equiv.) was dissolved in THF (43 mL) under an atmosphere of nitrogen. Potassium tert-butoxide (KO^{t}Bu, 7.0 g, 62.4 mmol, 7.10 equiv.) was added, and the reaction heated to reflux for 18 hours. The solvent was removed, and the crude residue was used in the next step without further purification.

The crude residue was suspended in methanol (60 mL), cooled to 0° C., and treated with concentrated sulfuric acid (H₂SO₄, 4.5 mL, 84 mmol, 9.6 equiv.) which was added slowly. The reaction was then heated to reflux for 18 hours. The reaction solvent was removed, and the reaction was quenched at room temperature with a saturated aqueous solution of NaHCO₃ (100 mL) followed by the addition of EtOAc (100 mL). The layers were separated; and the organic layer was dried with Na₂SO₄, filtered, and concentrated. The residue was adsorbed onto silica and subjected to FCC (EtOAc:hexanes:DCM:MeOH 10:90:0:0 to 100:0:0:0, then 0:0:80:20) to afford the title compound (1.84 g, 82% yield). LC-MS m/z 269 (M+H⁺).

### Step 4: Synthesis of methyl 5-bromo-1,2-dimethyl-1H-pyrrolo[2,3-b]pyridine-6-carboxylate

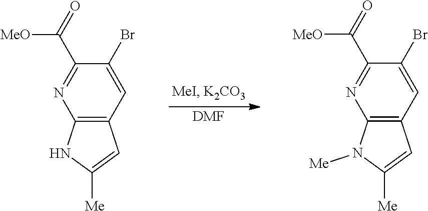

Methyl 5-bromo-2-methyl-1H-pyrrolo[2,3-b]pyridine-6-carboxylate (2.0 g, 7.4 mmol, 1.00 equiv.) was dissolved in DMF (22 mL) under an atmosphere of nitrogen. K₂CO₃ (3.1 g, 22 mmol, 3.0 equiv.) and MeI (1.4 mL, 22 mmol, 3.0 equiv.) were added, and the reaction was stirred at room temperature for 18 hours. EtOAc (200 mL) and water (200 mL) were added, and the layers were separated. The organic layer was collected, and the aqueous layer was reextracted with EtOAc (100 mL). The organic layers were combined and washed with brine (100 mL), dried over Na₂SO₄, filtered, and concentrated. The crude residue was adsorbed onto silica and subjected to FCC (EtOAc:hexanes 5:95 to 50:50) to afford the title compound (1.25 g, 59% yield). LC-MS m/z 283 (M+H⁺).

### Step 5: Synthesis of methyl 5-((diphenylmethylene)amino)-1,2-dimethyl-1H-pyrrolo[2,3-b]pyridine-6-carboxylate

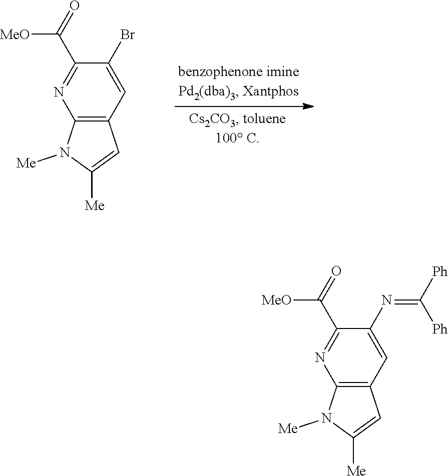 [SMILES: COC(=O)c1nc2c(cc1Br)cc(C)n2C ; COC(=O)c1nc2c(cc1N=C(c1ccccc1)c1ccccc1)cc(C)n2C]

Methyl 5-bromo-1,2-dimethyl-1H-pyrrolo[2,3-b]pyridine-6-carboxylate (1.25 g, 4.42 mmol, 1.0 equiv.), tris(dibenzylideneacetone)dipalladium(0) (Pd₂(dba)₃, 404 mg, 0.442 mmol, 0.10 equiv.), 4,5-bis(diphenylphosphino)-9,9-dimethylxanthene (Xantphos, 511 mg, 0.883 mmol, 0.20 equiv.), and Cs₂CO₃ (2.88 g, 8.84 mmol, 2.0 equiv.) were added to a microwave tube under an atmosphere of nitrogen. Toluene (26 mL) was added, and the reaction was degassed with nitrogen for 2 min. Benzophenone imine (1.11 mL, 6.61 mmol, 1.50 equiv.) was added, and the reaction was degassed for a further 5 mins. The reaction was heated in the microwave at 100° C. for 12 hours. The reaction was diluted with EtOAc (150 mL) and filtered through Celite®. The solvent was removed, and residue was adsorbed onto Celite® and subjected to FCC (MeOH:DCM 5:95 to 50:50) to afford the title compound (1.40 g, 83% yield). LC-MS m/z 384 (M+H⁺).

### Step 6: Synthesis of methyl 5-amino-3-cyano-1,2-dimethyl-1H-pyrrolo[2,3-b]pyridine-6-carboxylate

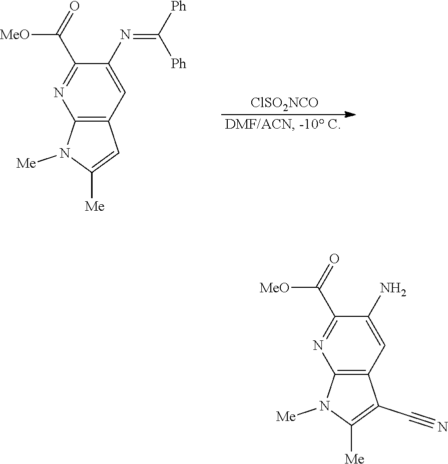

Methyl 5-((diphenylmethylene)amino)-1,2-dimethyl-1H-pyrrolo[2,3-b]pyridine-6-carboxylate (1.40 g, 3.65 mmol, 1.00 equiv.) was dissolved in DMF (14 mL) under an atmosphere of nitrogen and cooled to −10° C. To this was added a solution of chlorosulfonyl isocyanate (ClSO₂NCO, 1.30 mL, 15 mmol, 4.1 equiv.) in ACN (12 mL) dropwise over 5 minutes. The reaction was then allowed to warm to 0° C. and stirred for 1 hour. An aqueous solution of HCl (1M, 10 mL, 10 mmol, 2.74 equiv.) was added, and the reaction allowed to warm to room temperature. A saturated aqueous solution of NaHCO₃ was added until the pH>11, and then water (200 mL) and EtOAc (200 mL) were added, and the layers separated. The organic layer was collected, and the aqueous layer was reextracted with EtOAc (75 mL). The organic layers were combined, dried with MgSO₄, filtered, and concentrated. The residue was adsorbed onto silica and subjected to FCC (MeOH:DCM 0:100 to 20:80) to afford the title compound (590 mg, 66% yield). LC-MS m/z 245 (M+H⁺).

### Step 7: Synthesis of methyl 5-amino-4-bromo-3-cyano-1,2-dimethyl-1H-pyrrolo[2,3-b]pyridine-6-carboxylate

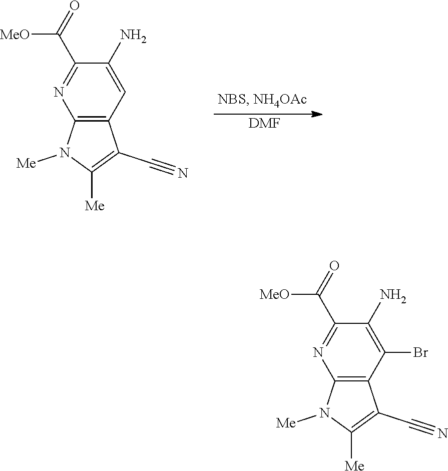

Methyl 5-amino-3-cyano-1,2-dimethyl-1H-pyrrolo[2,3-b]pyridine-6-carboxylate (606 mg, 2.48 mmol, 1.00 equiv.) was suspended in ACN (30 mL). Ammonium acetate (NH₄OAc, 20 mg, 0.259 mmol, 0.10 equiv.) was added, followed by a solution of NBS (463 mg, 2.601 mmol, 1.05 equiv.) in ACN (15 mL) dropwise over 5 minutes, and the reaction was stirred at room temperature for 30 minutes. The solvent was removed, and the residue adsorbed onto silica and subjected to FCC (MeOH:DCM 0:100 to 15:85). The fractions containing title compound were combined and transferred to a separatory funnel containing a saturated aqueous solution of NaHCO₃ (150 mL) and the layers were separated. The organic layer was collected, dried with Na₂SO₄, filtered, and concentrated to afford the title compound (160 mg, 20% yield). LC-MS m/z 323 (M+H⁺).

### Step 8: Synthesis of methyl 5-amino-3-cyano-4-(3-hydroxy-2-methylphenyl)-1,2-dimethyl-1H-pyrrolo[2,3-b]pyridine-6-carboxylate

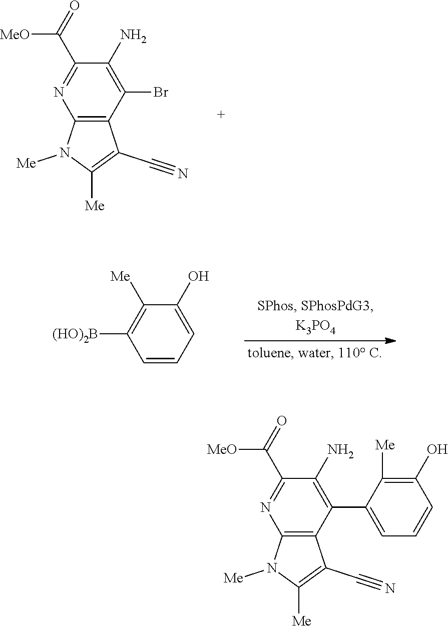 [SMILES: COC(=O)c1nc2c(c(Br)c1N)c(C#N)c(C)n2C ; Cc1c(O)cccc1B(O)O ; COC(=O)c1nc2c(c(C#N)c(C)n2C)c(-c2cccc(O)c2C)c1N]

A mixture of methyl 5-amino-4-bromo-3-cyano-1,2-dimethyl-1H-pyrrolo[2,3-b]pyridine-6-carboxylate (85 mg, 0.263 mmol, 1.00 equiv.), (3-methoxy-2,6-dimethylphenyl)boronic acid (100 mg, 0.658 mmol, 2.5 equiv.), dicyclohexyl(2′,6′-dimethoxy[1,1′-biphenyl]-2-yl)phosphane (SPhos, 22 mg, 0.054 mmol, 0.20 equiv.), K₃PO₄ (140 mg, 0.658 mmol, 2.5 equiv.) was suspended in toluene (2.0 mL) and water (0.2 mL). The mixture was degassed for 2 mins, then (2-dicyclohexylphosphino-2′,6′-dimethoxybiphenyl) [2-(2′-amino-1,1′-biphenyl)]palladium(II) methanesulfonate (SPhos Pd G3, 41 mg, 0.053 mmol, 0.20 equiv.) was added. The reaction was degassed for another 5 minutes. The reaction was heated in the microwave to 100° C. for 2 hours. The reaction was diluted with DCM (50 mL) and water (40 mL), and the layers were separated. The organic layer was collected, and the aqueous layer was reextracted 3× with DCM (40 mL). The organic layers were combined and dried with Na₂SO₄, filtered, and concentrated under reduced pressure. The residue was adsorbed onto silica and then subjected to FCC (MeOH:DCM 0:100 to 20:80) to afford the title compound (50 mg, 66% yield). LC-MS m/z 351 (M+H⁺).

### Step 9: Synthesis of 5-amino-3-cyano-4-(3-hydroxy-2-methylphenyl)-1,2-dimethyl-1H-pyrrolo[2,3-b]pyridine-6-carboxamide (Compound 9)

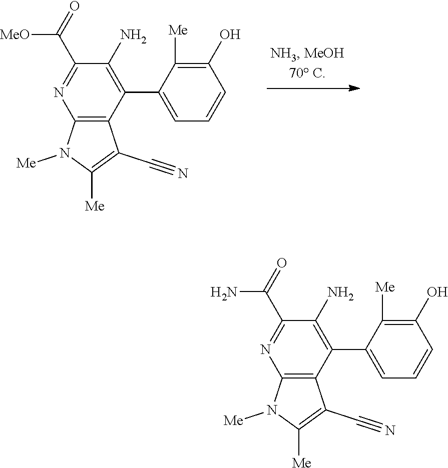 [SMILES: COC(=O)c1nc2c(c(C#N)c(C)n2C)c(-c2cccc(O)c2C)c1N ; Cc1c(O)cccc1-c1c(N)c(C(N)=O)nc2c1c(C#N)c(C)n2C]

Methyl 5-amino-3-cyano-4-(3-hydroxy-2-methylphenyl)-1,2-dimethyl-1H-pyrrolo[2,3-b]pyridine-6-carboxylate (50 mg, 0.14 mmol, 1.0 equiv.) was dissolved in a solution of ammonia in MeOH (7M, 12 mL, 77 mmol, 159 equiv.) in a microwave tube. The reaction was heated to 70° C. for 15 hours. The solvent was removed, and the crude residue was subjected to RP-HPLC (0.1% FA, Water:MeCN, 95:5 to 0:100) to afford the title compound (6.2 mg, 7% yield). 1H NMR (400 MHz, DMSO) δ 9.52 (s, 1H), 8.32 (s, 1H), 7.47 (s, 1H), 7.11 (t, J=7.1 Hz, 1H), 6.88 (s, 1H), 6.56 (d, J=7.4 Hz, 1H), 5.92 (s, 2H), 3.80 (d, J=3.0 Hz, 3H), 1.79 (d, J=2.9 Hz, 3H), CH₃ signal obscured by residual DMSO at 2.50 ppm. LC-MS m/z 336 (M+H⁺).

### Example 8. (M)-5-Amino-4-(5-chloro-4-methylpyridin-3-yl)-3-cyano-1-methyl-1H-pyrrolo[2,3-b]pyridine-6-carboxamide (10) and (P)-5-amino-4-(5-chloro-4-methylpyridin-3-yl)-3-cyano-1-methyl-1H-pyrrolo[2,3-b]pyridine-6-carboxamide (11)

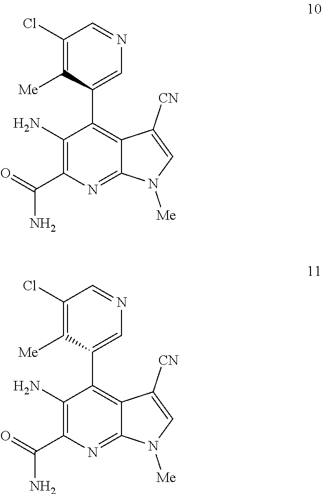

Compounds 10 and 11 were prepared by analogy to Example 3 using 3-chloro-4-methyl-5-(4,4,5,5-tetramethyl-1,3,2-dioxaborolan-2-yl)pyridine in Step 5 to obtain the title compounds as a racemic mixture that was subjected to chiral SFC separation.

Instrument: Waters SFC Prep 150 Mgm; Column: OD, 30×250 mm, 5 um; Conditions: Isocratic at 40% methanol with 60% CO₂: Flow Rate: 100 mL/min) provided compound 10 (14.3 mg) and compound 11 (14.6 mg).

Peak 1: SFC t_{R}=2.62 min. ¹H NMR (400 MHz, d6-DMSO) δ 8.70 (s, 1H), 8.43 (s, 1H), 8.38 (d, J=2.8 Hz, 1H), 8.32 (s, 1H), 7.63 (d, J=2.9 Hz, 1H), 6.44 (s, 2H), 3.89 (s, 3H), 2.09 (s, 3H). LC-MS m/z 341 (M+H⁺).

Peak 2: SFC t_{R}=3.29 min. ¹H NMR (400 MHz, d6-DMSO) δ 8.43 (s, 1H), 8.38 (d, J=2.7 Hz, 1H), 8.32 (s, 1H), 7.63 (d, J=2.8 Hz, 1H), 6.44 (s, 2H), 3.89 (s, 3H), 2.09 (s, 3H). LC-MS m/z 341 (M+H⁺).

### Example 9. 5-Amino-4-(5-chloro-4-methylpyridin-3-yl)-3-cyano-1,2-dimethyl-1H-pyrrolo[2,3-b]pyridine-6-carboxamide (12)

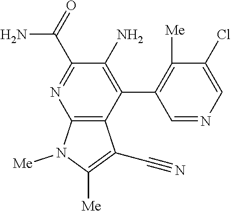 [SMILES: Cc1c(Cl)cncc1-c1c(N)c(C(N)=O)nc2c1c(C#N)c(C)n2C]

Prepared according to Example 7, step 8 using (5-chloro-4-methylpyridin-3-yl)boronic acid to give the title compound (4.8 mg, 7% yield); LC-MS m/z 355.2 [M+H⁺]; 1H NMR (400 MHz, DMSO) δ 8.68 (s, 1H), 8.35 (d, J=2.9 Hz, 1H), 8.28 (s, 1H), 7.51 (d, J=2.8 Hz, 1H), 6.38 (s, 2H), 3.82 (s, 3H), 2.08 (s, 3H). A CH₃ was obscured by residual DMSO signal at 2.50 ppm.

### Example 10. 5-Amino-3-cyano-4-(3-hydroxy-2,6-dimethylphenyl)-1-methyl-1H-pyrrolo[2,3-b]pyridine-6-carboxamide (13)

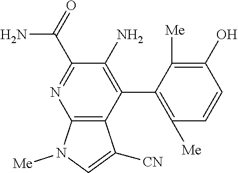 [SMILES: Cc1ccc(O)c(C)c1-c1c(N)c(C(N)=O)nc2c1c(C#N)cn2C]

Compound 13 was synthesized by analogy to Example 3, using (3-methoxy-2,6-dimethylphenyl)boronic acid as the reactant and tris(dibenzylideneacetone)dipalladium(0) (Pd₂(dba)₃ as the catalyst in Step 5. Purification with RP-HPLC (0.1% FA, Water:MeCN, 95:5 to 0:100) afforded the title compound (34 mg, 41% yield). ¹H NMR (400 MHz, DMSO) δ 9.21 (s, 1H), 8.35 (s, 1H), 8.30 (d, J=3.0 Hz, 1H), 7.54 (d, J=3.1 Hz, 1H), 6.99 (d, J=8.2 Hz, 1H), 6.80 (d, J=8.2 Hz, 1H), 5.88 (s, 2H), 3.87 (s, 3H), 1.74 (d, J=24.2 Hz, 6H). LC-MS m/z 336 (M+H⁺).

### Example 11. 5-Amino-3-cyano-4-(3-hydroxy-2-methylphenyl)-2-methyl-1-(2,2,2-trifluoroethyl)-1H-pyrrolo[2,3-b]pyridine-6-carboxamide (14)

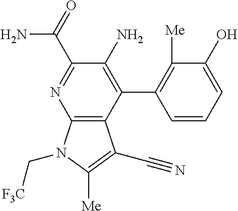 [SMILES: Cc1c(O)cccc1-c1c(N)c(C(N)=O)nc2c1c(C#N)c(C)n2CC(F)(F)F]

Prepared according to Example 7, step 4 using 2,2,2-trifluoroethyl trifluoromethanesulfonate instead of MeI to give the title compound (26.7 mg); LC-MS m/z 404.1 [M+H⁺]; ¹H NMR (400 MHz, DMSO) δ 9.50 (s, 1H), 8.51 (d, J=2.9 Hz, 1H), 7.48 (d, J=2.9 Hz, 1H), 7.12 (t, J=7.8 Hz, 1H), 6.91 (dd, J=8.1, 1.2 Hz, 1H), 6.60 (dd. J=7.5, 1.2 Hz, 1H), 6.01 (s, 2H), 5.56-5.28 (m, 2H), 2.55 (s, 3H), 1.81 (s, 3H).

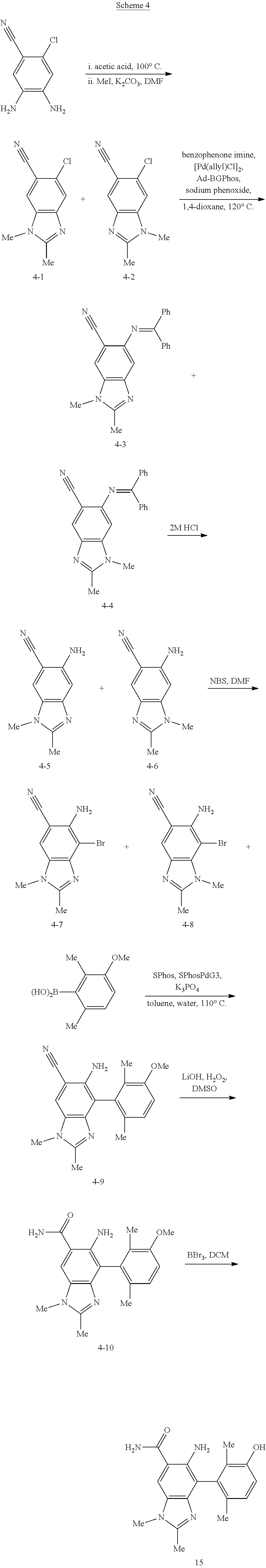

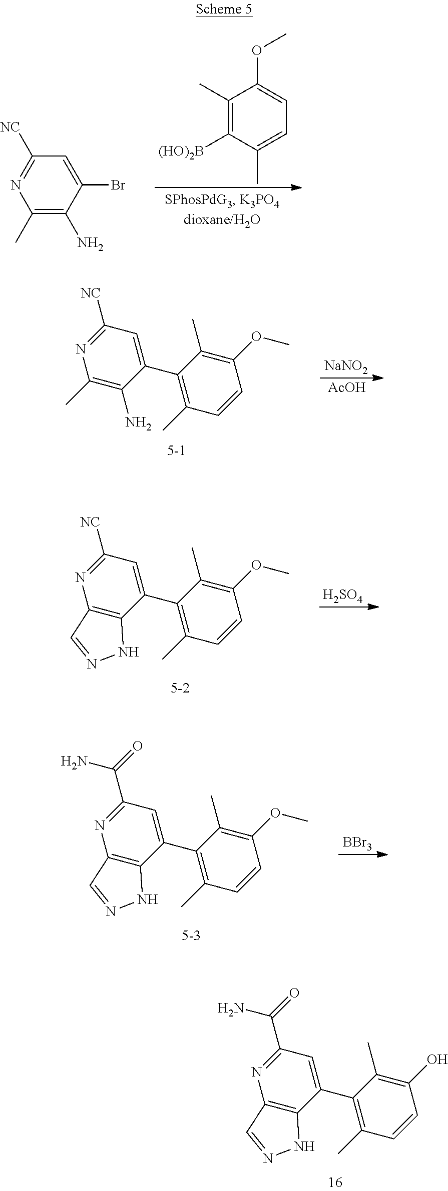

### Example 12. 5-Amino-4-(3-hydroxy-2,6-dimethylphenyl)-1,2-dimethyl-1H-benzo[d]imidazole-6-carboxamide (15)

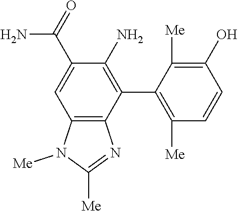 [SMILES: Cc1ccc(O)c(C)c1-c1c(N)c(C(N)=O)cc2c1nc(C)n2C]

### Step 1: Synthesis of 6-chloro-1,2-dimethyl-1H-benzo[d]imidazole-5-carbonitrile and 5-chloro-1,2-dimethyl-1H-benzo[d]imidazole-6-carbonitrile

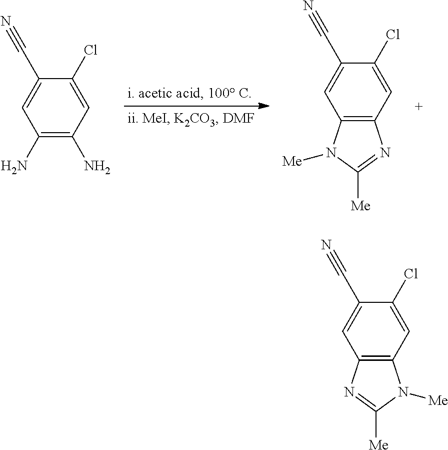 [SMILES: N#Cc1cc(N)c(N)cc1Cl ; Cc1nc2cc(Cl)c(C#N)cc2n1C ; Cc1nc2cc(C#N)c(Cl)cc2n1C]

4,5-Diamino-2-chlorobenzonitrile (1.00 g, 5.97 mmol, 1.00 equiv.) was added to acetic acid (20 mL, 350 mmol, 58.6 equiv.) and the mixture was heated to 110° C. for 18 hours. The solvent was removed by rotary evaporation to yield 5-chloro-2-methyl-1H-benzo[d]imidazole-6-carbonitrile, LC-MS m/z 192 (M+H⁺), which was used in the next step without further purification.

The crude residue was dissolved in DMF (13 mL) under an atmosphere of nitrogen, followed by K₂CO₃ (1.50 g, 10.9 mmol, 1.82 equiv.) and MeI (0.69 mL, 11 mmol, 1.9 equiv.) and the reaction was stirred at 40° C. for 2 hours. The solvent was removed, the residue adsorbed into Celite®, then subjected to FCC (MeOH:DCM, 0:100 to 10:90) to afford the title compounds (850 mg, 69% yield) as a 1:1 mixture of isomers. LC-MS m/z 206 (M+H⁺).

### Step 2: Synthesis of 5-((diphenylmethylene)amino)-1,2-dimethyl-1H-benzo[d]imidazole-6-carbonitrile and 6-((diphenylmethylene)amino)-1,2-dimethyl-1H-benzo[d]imidazole-5-carbonitrile

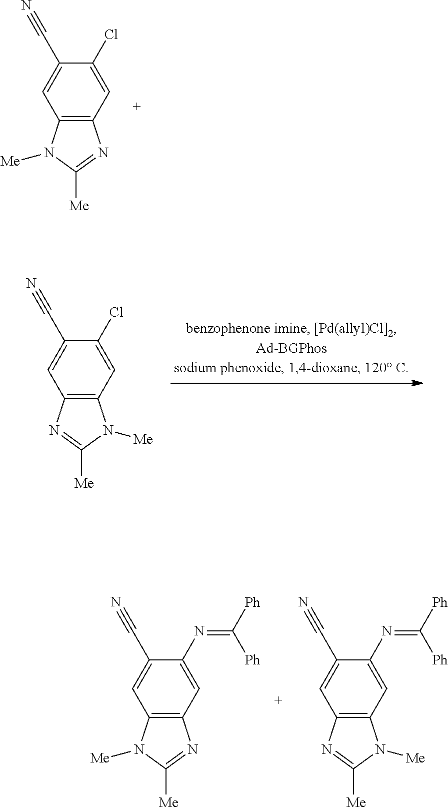 [SMILES: Cc1nc2cc(Cl)c(C#N)cc2n1C ; Cc1nc2cc(C#N)c(Cl)cc2n1C ; Cc1nc2cc(N=C(c3ccccc3)c3ccccc3)c(C#N)cc2n1C ; Cc1nc2cc(C#N)c(N=C(c3ccccc3)c3ccccc3)cc2n1C]

A 1:1 mixture of 6-chloro-1,2-dimethyl-1H-benzo[d]imidazole-5-carbonitrile and 5-chloro-1,2-dimethyl-1H-benzo[d]imidazole-6-carbonitrile (800 mg, 3.89 mmol, 1.00 equiv.), benzophenone imine (1.00 mL, 5.96 mmol, 1.53 equiv.), 5-[Di(1-adamantyl)phosphino]-1′,3′,5′-triphenyl-1′H-[1,4′]bipyrazole (Ad-BGPhos, 80 mg, 0.12 mmol, 0.031 equiv.), sodium phenoxide (903 mg, 7.78 mmol, 2.00 equiv.) and 1,4-dioxane (12 mL) under an atmosphere of nitrogen. The reaction was degassed with nitrogen for 2 minutes, followed by the addition of allylpalladium(II) chloride dimer ([Pd(allyl)Cl]₂, 90 mg, 0.25 mmol, 0.063 equiv.) and the reaction was degassed with nitrogen for another 5 minutes. The reaction was heated in the microwave at 120° C. for 3 hours. The solvent was removed, the residue adsorbed onto Celite®, then subjected to FCC (EtOAc:hexane 5:95 to 50:50) to afford the title compounds (670 mg, 49% yield) and a 1:1 mixture of isomers. LC-MS m/z 351 (M+H⁺).

### Step 3: Synthesis of 5-amino-1,2-dimethyl-1H-benzo[d]imidazole-6-carbonitrile and 6-amino-1,2-dimethyl-1H-benzo[d]imidazole-5-carbonitrile

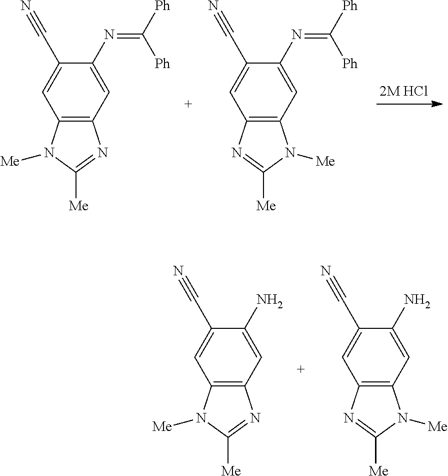 [SMILES: Cc1nc2cc(N=C(c3ccccc3)c3ccccc3)c(C#N)cc2n1C ; Cc1nc2cc(C#N)c(N=C(c3ccccc3)c3ccccc3)cc2n1C ; Cc1nc2cc(N)c(C#N)cc2n1C ; Cc1nc2cc(C#N)c(N)cc2n1C]

A 1:1 mixture of 5-((diphenylmethylene)amino)-1,2-dimethyl-1H-benzo[d]imidazole-6-carbonitrile and 6-((diphenylmethylene)amino)-1,2-dimethyl-1H-benzo[d]imidazole-5-carbonitrile (670 mg, 1.91 mmol, 1.00 equiv.) was suspended in THF (6.6 mL) followed by the addition of 2M HCl in water (1.33 mL) and stirred at room temperature for 30 minutes. The reaction was diluted with a 2:1 mixture of Hexanes:EtOAc (50 mL) and 0.5M HCl in water (50 mL). The layers were separated, and the aqueous layer was collected and basified with a saturated Na₂CO₃ solution until pH>11. DCM (70 mL) was added, and the layers separated. The organic layer was collected, and the aqueous layer was reextracted 4× with DCM (70 mL). The organic layers were combined, dried with Na₂SO₄, filtered, and concentrated to yield the title compounds (250 mg, 70% yield) as a 1:1 mixture of isomers. LC-MS m/z 187 (M+H⁺).

### Step 4: Synthesis of 6-amino-7-bromo-1,2-dimethyl-1H-benzo[d]imidazole-5-carbonitrile and 5-amino-4-bromo-1,2-dimethyl-1H-benzo[d]imidazole-6-carbonitrile

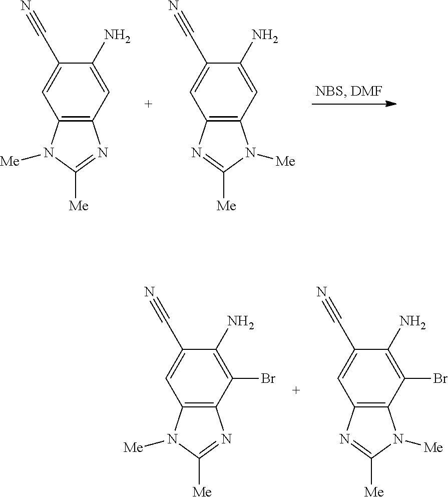 [SMILES: Cc1nc2cc(N)c(C#N)cc2n1C ; Cc1nc2cc(C#N)c(N)cc2n1C ; Cc1nc2c(Br)c(N)c(C#N)cc2n1C ; Cc1nc2cc(C#N)c(N)c(Br)c2n1C]

A 1:1 mixture of 5-amino-1,2-dimethyl-1H-benzo[d]imidazole-6-carbonitrile and 6-amino-1,2-dimethyl-1H-benzo[d]imidazole-5-carbonitrile (250 mg, 1.34 mmol, 1.00 equiv.) was dissolved in DMF (2.5 mL) under an atmosphere of nitrogen. NBS (286 mg, 1.60 mmol, 1.20 equiv.) was added and the reaction stirred at room temperature for 30 minutes. The solvent was removed. The residue was adsorbed onto Celite®, then subjected to FCC (EtOAc:Hexanes 50:50 to 100:0) to afford the title compounds (125 mg, 35% yield) as a 4:1 mixture of isomers. NMR spectroscopy determined that the major isomer was 5-amino-4-bromo-1,2-dimethyl-1H-benzo[d]imidazole-6-carbonitrile. LC-MS m/z 265 (M+H⁺).

### Step 5: Synthesis of 5-amino-4-(3-methoxy-2,6-dimethylphenyl)-1,2-dimethyl-1H-benzo[d]imidazole-6-carbonitrile

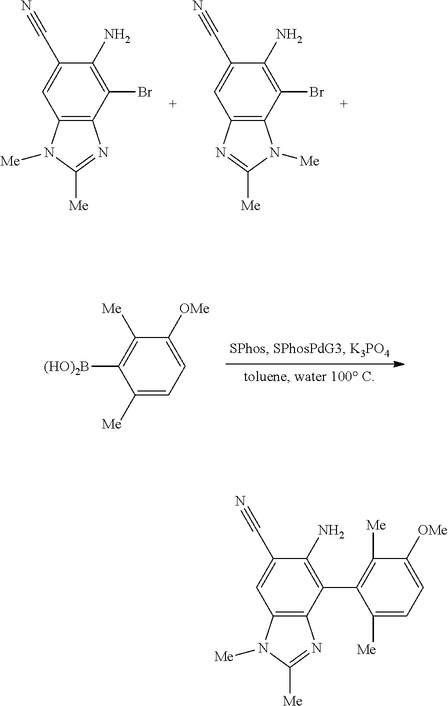 [SMILES: Cc1nc2c(Br)c(N)c(C#N)cc2n1C ; Cc1nc2cc(C#N)c(N)c(Br)c2n1C ; COc1ccc(C)c(B(O)O)c1C ; COc1ccc(C)c(-c2c(N)c(C#N)cc3c2nc(C)n3C)c1C]

A 4:1 mixture of 6-amino-7-bromo-1,2-dimethyl-1H-benzo[d]imidazole-5-carbonitrile and 5-amino-4-bromo-1,2-dimethyl-1H-benzo[d]imidazole-6-carbonitrile (220 mg, 0.829 mmol, 1.00 equiv.), (3-methoxy-2,6-dimethylphenyl)boronic acid (299 mg, 1.66 mmol, 2.00 equiv.), dicyclohexyl(2′,6′-dimethoxy[1,1′-biphenyl]-2-yl)phosphane (SPhos, 68 mg, 0.16 mmol, 0.20 equiv.), and K₃PO₄ (352 mg, 1.66 mmol, 2.00 equiv.) was suspended in toluene (2.6 mL) and water (0.3 mL). The reaction was degassed for 2 min, then (2-dicyclohexylphosphino-2′,6′-dimethoxybiphenyl) [2-(2′-amino-1,1′-biphenyl)]palladium(II) methanesulfonate (SPhos Pd G3, 130 mg, 0.16 mmol, 0.20 equiv.) was added. The reaction was degassed for another 5 minutes. The reaction was heated in the microwave to 100° C. for 6 hours. The reaction was diluted with DCM (50 mL) and water (40 mL), and the layers were separated. The organic layer was collected, and the aqueous layer was reextracted 3× with DCM. The organic layers were combined and dried with Na₂SO₄, filtered, and concentrated. The residue was adsorbed onto silica and then subjected to FCC (MeOH:DCM 0:100 to 10:90) to afford the title compound (120 mg, 45% yield) as a red foam. LC-MS m/z 321 (M+H⁺).

### Step 6: Synthesis of 5-amino-4-(3-methoxy-2,6-dimethylphenyl)-1,2-dimethyl-1H-benzo[d]imidazole-6-carboxamide

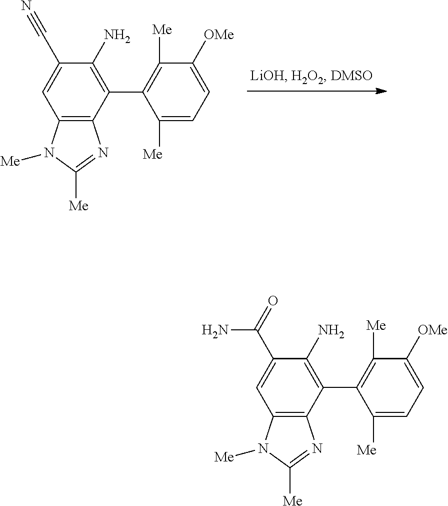 [SMILES: COc1ccc(C)c(-c2c(N)c(C#N)cc3c2nc(C)n3C)c1C ; COc1ccc(C)c(-c2c(N)c(C(N)=O)cc3c2nc(C)n3C)c1C]

5-Amino-4-(3-methoxy-2,6-dimethylphenyl)-1,2-dimethyl-1H-benzo[d]imidazole-6-carbonitrile (120 mg, 0.375 mmol, 1.00 equiv.) was dissolved in DMSO (1.0 mL) and water (0.3 mL). LiOH monohydrate (40 mg, 0.95 mmol, 2.5 equiv.) was added followed by 30% (w/w) H₂O₂ in water (0.10 mL, 0.88 mmol, 2.4 equiv.), and the reaction was stirred at room temperature for 20 minutes. The reaction was diluted with EtOAc (50 mL) and water (45 mL), and the layers were separated. The organic layer was collected, and the aqueous layer was reextracted with EtOAc. The organic layers were combined and dried with Na₂SO₄, filtered, and concentrated. The residue was subjected to FCC (MeOH:DCM 0:100 to 15:85) to afford the title compound (60 mg, 47% yield). LC-MS m/z 339 (M+H⁺).

### Step 7: Synthesis of 5-amino-4-(3-hydroxy-2,6-dimethylphenyl)-1,2-dimethyl-1H-benzo[d]imidazole-6-carboxamide (compound 15)

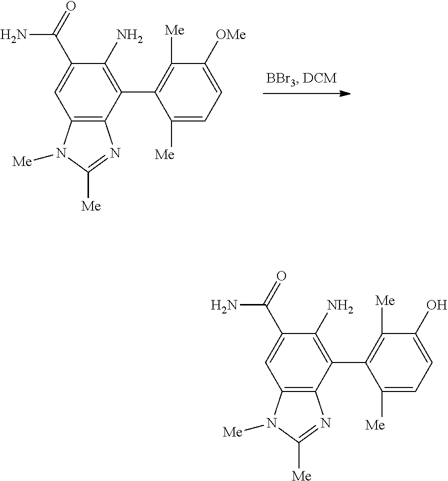 [SMILES: COc1ccc(C)c(-c2c(N)c(C(N)=O)cc3c2nc(C)n3C)c1C ; Cc1ccc(O)c(C)c1-c1c(N)c(C(N)=O)cc2c1nc(C)n2C]

5-amino-4-(3-methoxy-2,6-dimethylphenyl)-1,2-dimethyl-1H-benzo[d]imidazole-6-carboxamide (60 mg, 0.177 mmol, 1.00 equiv.) was dissolved in DCM (4 mL) under an atmosphere of nitrogen gas and cooled to 0° C. A solution of BBr₃ in DCM (1 M, 0.88 mL, 5.00 equiv.) was added dropwise over 1 min and the mixture was stirred 0° C. for 2 hours. Methanol (2 mL) was added, and the solvent was removed. The residue was dissolved in DMF, filtered, and subjected to RP-HPLC (0.1% FA. Water:MeCN, 90:10 to 0:100) to afford the title compound (9.1 mg, 16% yield). ¹H NMR (400 MHz, DMSO) δ 9.00 (s, 1H), 7.78 (s, 1H), 7.69 (d, J=2.5 Hz, 1H), 7.13 (s, 1H), 6.87 (d, J=8.1 Hz, 1H), 6.68 (d, J=8.0 Hz, 1H), 5.28 (s, 2H), 3.60 (d, J=2.7 Hz, 3H), 2.33 (d, J=2.6 Hz, 3H), 1.67 (d, J=2.5 Hz, 3H), 1.60 (d, J=2.5 Hz, 3H). LC-MS m/z 325 (M+H⁺).

### Example 13. 7-(3-Hydroxy-2,6-dimethylphenyl)-1H-pyrazolo[4,3-b]pyridine-5-carboxamide (16)

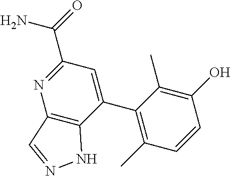 [SMILES: Cc1ccc(O)c(C)c1-c1cc(C(N)=O)nc2cn[nH]c12]

### Step 1: Synthesis of 5-amino-4-(3-methoxy-2,6-dimethylphenyl)-6-methyl-pyridine-2-carbonitrile

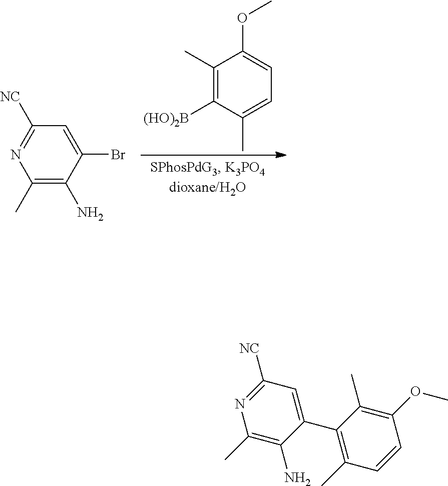

A mixture of 5-amino-4-bromo-6-methyl-pyridine-2-carbonitrile (700 mg, 3.30 mmol, 1 eq), (3-methoxy-2,6-dimethylphenyl)boronic acid (891 mg, 4.95 mmol, 1.5 eq), SPhos Pd G₃ (258 mg, 0.330 mmol, 0.1 eq). K₃PO₄ (2.10 g, 9.90 mmol, 3 eq) in dioxane (12 mL) and H₂O (3 mL) was degassed with N₂ and stirred at 100° C. for 3 hours. The reaction mixture was diluted with H₂O (10 mL) and extracted with EtOAc (20 mL×3). The combined organic layers were washed with aq. NaCl (10 mL×2), dried over anhyd. Na₂SO₄, filtered and concentrated to give a residue. The residue was purified by column chromatography (SiO₂, 0~15% Ethyl acetate in Petroleum ether) to give 5-amino-4-(3-methoxy-2,6-dimethylphenyl)-6-methyl-pyridine-2-carbonitrile (510 mg, 1.91 mmol, 57.9% yield). LC-MS m/z 268 (M+H⁺).

### Step 2: Synthesis of 7-(3-methoxy-2,6-dimethylphenyl)-1H-pyrazolo[4,3-b]pyridine-5-carbonitrile

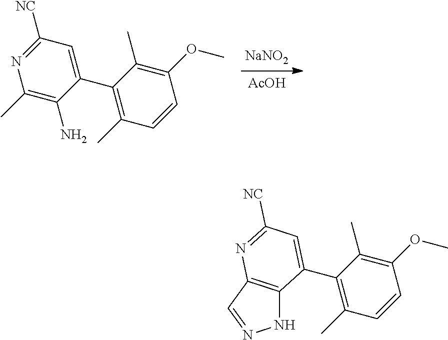

To a solution of 5-amino-4-(3-methoxy-2,6-dimethylphenyl)-6-methyl-pyridine-2-carbonitrile (510 mg, 1.91 mmol, 1 eq) in AcOH (8 mL) was added a solution of NaNO₂ (145 mg, 2.10 mmol, 1.1 eq) in H₂O (0.8 mL) at 0° C. The mixture was stirred at 20° C. for 2 hours. The reaction mixture was quenched by aq. saturated Na₂S₂O₃ (15 mL), diluted with H₂O (10 mL) and extracted with EtOAc (20 mL×3). The combined organic layers were washed with aq. NaCl (10 mL×2), dried over Na₂SO₄, filtered, and concentrated to give a residue. The residue was purified by column chromatography (SiO₂, 0~14% Ethyl acetate in Petroleum ether) to give 7-(3-methoxy-2,6-dimethylphenyl)-1H-pyrazolo[4,3-b]pyridine-5-carbonitrile (210 mg, 0.68 mmol, 36% yield, 90% purity) was obtained. LC-MS m/z 279 (M+H⁺).

### Step 3: Synthesis of 7-(3-methoxy-2,6-dimethylphenyl)-1H-pyrazolo[4,3-b]pyridine-5-carboxamide

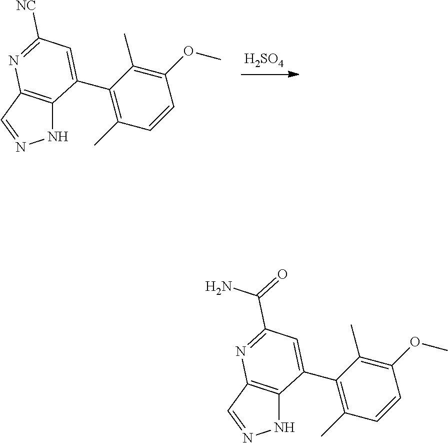

A mixture of 7-(3-methoxy-2,6-dimethylphenyl)-1H-pyrazolo[4,3-b]pyridine-5-carbonitrile (210 mg, 0.76 mmol, 1 eq) in concentrated H₂SO₄ (4 mL) was stirred at 25° C. for 12 hours. The reaction mixture was adjusted to pH 7 by aq. NH₃·H₂O. The mixture was filtered to give the filter cake, which was dried in vacuo to give 7-(3-methoxy-2,6-dimethylphenyl)-1H-pyrazolo[4,3-b]pyridine-5-carboxamide (85 mg, 0.27 mmol, 35% yield, 93% purity). LC-MS m/z 297 (M+H⁺).

### Step 4: Synthesis of 7-(3-hydroxy-2,6-dimethylphenyl)-1H-pyrazolo[4,3-b]pyridine-5-carboxamide (compound 16)

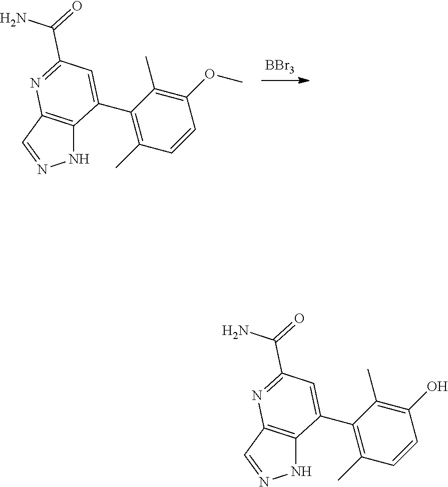

To a solution of 7-(3-methoxy-2,6-dimethylphenyl)-1H-pyrazolo[4,3-b]pyridine-5-carboxamide (85 mg, 0.29 mmol, 1 eq) in DCM (3 mL) was added BBr₃ (0.15 mL, 5 eq) at 0° C. The mixture was stirred at 25° C. for 1 hour. The reaction mixture was added dropwise into water (10 mL) at 25° C., then concentrated to give a residue. The residue was purified by RP-HPLC (column: Welch Xtimate C18 150×30 mm×5 um; mobile phase: [water(FA)-ACN]; B %: 0%-38%, 30 min) to give 7-(3-hydroxy-2,6-dimethylphenyl)-1H-pyrazolo[4,3-b]pyridine-5-carboxamide (27.8 mg, 0.10 mmol, 34% yield, 98% purity). LC-MS m/z 283 (M+H⁺). ¹H NMR (400 MHz, DMSO-d₆) δ=13.44 (s, 1H), 9.39 (s, 1H), 8.45 (s, 1H), 8.20 (d, 1H), 7.73 (s, 1H), 7.63 (d, 1H), 7.03 (d, 1H), 6.87 (d, 1H), 1.80 (s, 3H), 1.72 (s, 3H).

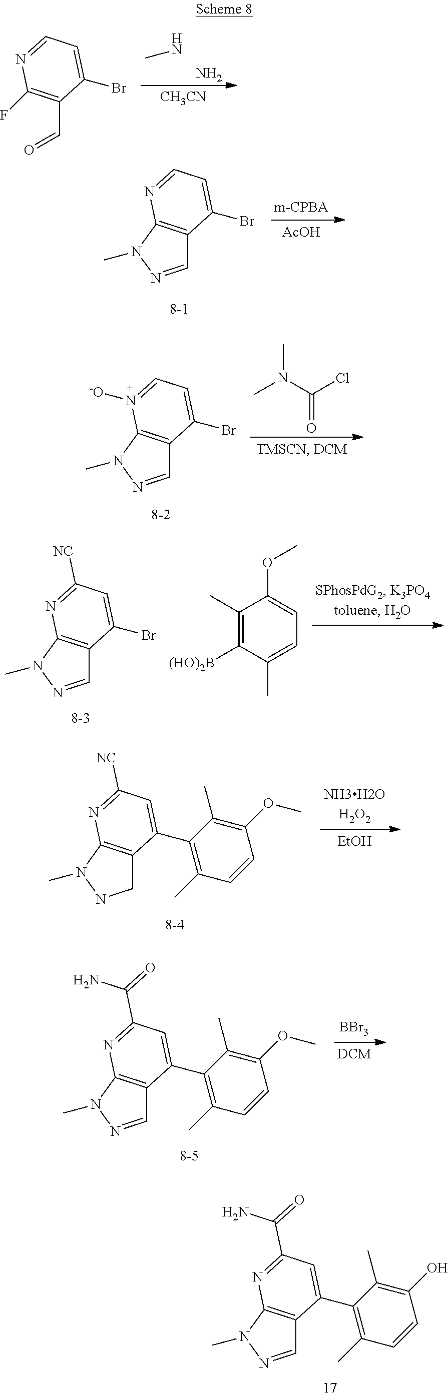

### Example 14. 4-(3-Hydroxy-2,6-dimethylphenyl)-1-methyl-1H-pyrazolo[3,4-b]pyridine-6-carboxamide (17)

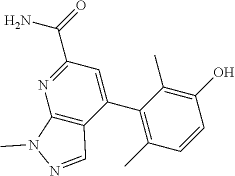 [SMILES: Cc1ccc(O)c(C)c1-c1cc(C(N)=O)nc2c1cnn2C]

### Step 1: Synthesis of 4-bromo-1-methyl-pyrazolo[3,4-b]pyridine

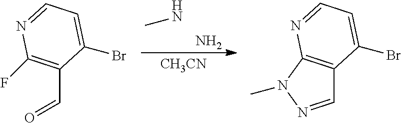

A mixture of 4-bromo-2-fluoro-pyridine-3-carbaldehyde (950 mg, 4.66 mmol, 1 eq), methylhydrazine (1.41 g, 12.24 mmol, 1.61 mL, 40% purity, 2.63 eq) and DIEA (1.20 g, 9.31 mmol, 1.62 mL, 2 eq) in CH₃CN (10 mL) was stirred at 60° C. for 6 hrs. The mixture was diluted with EtOAc (50 mL) and washed with water (100 mL). The aqueous layer was extracted with EtOAc (300 mL). The combined extracts were washed with brine (20 mL), dried with Na₂SO₄, filtered, and concentrated in vacuo to give a residue. The residue was purified by column chromatography (SiO₂, Petroleum ether/Ethyl acetate=4/1) to give 4-bromo-1-methyl-pyrazolo[3,4-b]pyridine (0.78 g, 79% yield). LC-MS m/z 212 (M+H⁺).

### Step 2: Synthesis of 4-bromo-1-methyl-7-oxido-pyrazolo[3,4-b]pyridin-7-ium

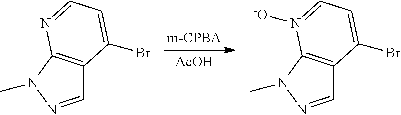

To a mixture of 4-bromo-1-methyl-pyrazolo[3,4-b]pyridine (500 mg, 2.36 mmol, 1 eq) in HOAc (20 mL) was added m-CPBA (3.05 g, 14.15 mmol, 80% purity, 6 eq). Then the mixture was stirred at 25° C. for 4 days. The reaction mixture was concentrated under reduced pressure to give a residue. The residue was purified by column chromatography (SiO₂, DCM:MeOH=20:1 (1% NH₃H₂O)) to give 4-bromo-1-methyl-7-oxido-pyrazolo[3,4-b]pyridin-7-ium (338 mg, crude). LC-MS m/z 228 (M+H⁺).

### Step 3: Synthesis of 4-bromo-1-methyl-pyrazolo[3,4-b]pyridine-6-carbonitrile

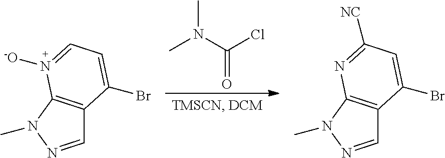

To a solution of 4-bromo-1-methyl-7-oxido-pyrazolo[3,4-b]pyridin-7-ium (310 mg, 1.36 mmol, 1 eq) in DCM (5 mL) was added TMSCN (269.2 mg, 2.71 mmol, 339.47 μL, 2 eq). After 15 minutes, N,N-dimethylcarbamoyl chloride (292.7 mg, 2.72 mmol, 250.17 μL, 2 eq) was added over a 15-minute period. The reaction was stirred for 4 h, and additional amount of trimethylsilylformonitrile (269.2 mg, 2.71 mmol, 339.47 μL, 2 eq) and N. N-dimethylcarbamoyl chloride (292.7 mg, 2.72 mmol, 250.17 μL, 2 eq) were added. The mixture was stirred at 25° C. for 18 hours. The reaction was quenched with 10% K₂CO₃ (30 mL), washed with water and back extracted twice with DCM (100 mL). The organic layers were combined and dried with Na₂SO₄, filtered, and concentrated in vacuo to give a residue. The residue was purified by column chromatography (SiO₂. Petroleum ether/Ethyl acetate=3/1) to give 4-bromo-1-methyl-pyrazolo[3,4-b]pyridine-6-carbonitrile (166 mg, 52% yield). LC-MS m/z 237 (M+H⁺).

### Step 4: Synthesis of 4-(3-methoxy-2,6-dimethylphenyl)-1-methyl-pyrazolo[3,4-b]pyridine-6-carbonitrile

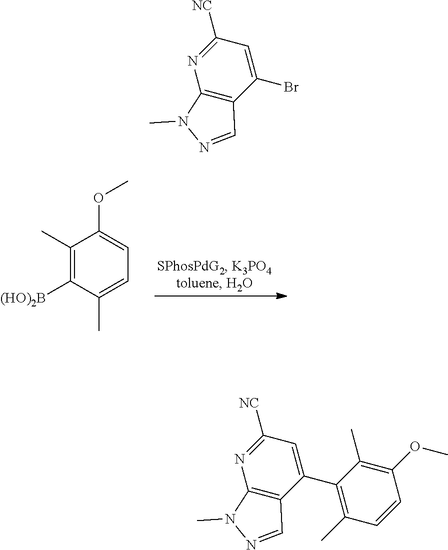

To a solution of 4-bromo-1-methyl-pyrazolo[3,4-b]pyridine-6-carbonitrile (150 mg, 632.8 μmol, 1 eq), (3-methoxy-2,6-dimethylphenyl)boronic acid (170.9 mg, 949.14 μmol, 1.5 eq), K₃PO₄ (402.9 mg, 1.90 mmol, 3 eq) in water (0.1 mL) and toluene (1 mL) was added dicyclohexyl-[2-(2,6-dimethoxyphenyl)phenyl]phosphane; methanesulfonate; (2-phenylanilino)palladium (49.4 mg, 63.28 μmol, 0.1 eq). The mixture was stirred at 105° C. for 10 hrs under N₂. The reaction mixture was filtered and concentrated under reduced pressure to give a residue. The residue was purified by column chromatography (SiO₂, Petroleum ether/Ethyl acetate=3/1) to give 4-(3-methoxy-2,6-dimethylphenyl)-1-methyl-pyrazolo[3,4-b]pyridine-6-carbonitrile (0.09 g, 49% yield). LC-MS m/z 293 (M+H⁺).

### Step 5: Synthesis of 4-(3-methoxy-2,6-dimethylphenyl)-1-methyl-pyrazolo[3,4-b]pyridine-6-carboxamide

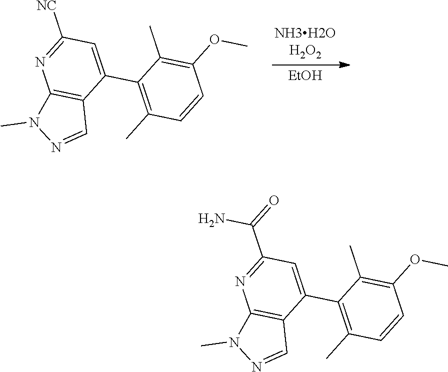

To a solution of 4-(3-methoxy-2,6-dimethylphenyl)-1-methyl-pyrazolo[3,4-b]pyridine-6-carbonitrile (65 mg, 222.35 μmol, 1 eq) in EtOH (1 mL) was added dropwise NH₃·H₂O (1.37 g, 13.63 mmol, 1.5 mL, 35% purity, 61 eq) and H₂O₂ (7.67 g, 67.65 mmol, 6.50 mL, 30% purity, 304 eq). The mixture was stirred at 25° C. for 24 hr. The reaction mixture was diluted with water (2 mL) and extracted with EtOAc (10 mL). Solution was filtered and concentrated under reduced pressure to give 4-(3-methoxy-2,6-dimethylphenyl)-1-methyl-pyrazolo[3,4-b]pyridine-6-carboxamide (68 mg, crude). LC-MS m/z 311 (M+H⁺).

### Step 6: Synthesis of 4-(3-hydroxy-2,6-dimethylphenyl)-1-methyl-pyrazolo[3,4-b]pyridine-6-carboxamide (compound 17)

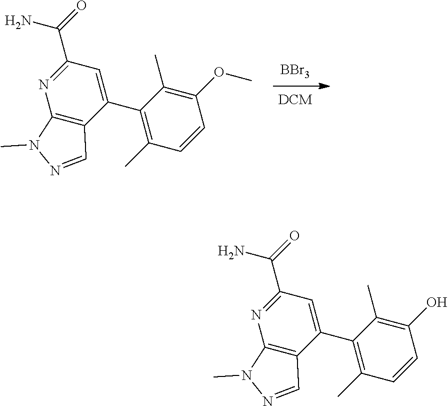

To a solution of 4-(3-methoxy-2,6-dimethylphenyl)-1-methyl-pyrazolo[3,4-b]pyridine-6-carboxamide (65 mg, 209 μmol, 1 eq) in DCM (1 mL) was added dropwise BBr₃ (157.4 mg, 628 μmol, 60.54 μL, 3 eq) at 0° C. for 5 min. The mixture was stirred at 25° C. for 4 hr. The reaction mixture was poured into ice water (5 mL) and then extracted with dichloromethane (30 mL), and the organic phase was washed with saturated brine (3 mL), dried and evaporated in vacuum to give a residue. The mixture was purification by RP-HPLC (column: Phenomenex C18 80×40 mm×3 um; mobile phase: [water (NH₃H₂O+NH₄HCO₃)-ACN]; B %: 34%-64%, 8 min) to give 4-(3-hydroxy-2,6-dimethylphenyl)-1-methyl-pyrazolo[3, 4-b]pyridine-6-carboxamide (0.05 g, 168.7 μmol, 80% yield). LC-MS m/z 297 (M+H⁺). ¹H NMR (400 MHz, DMSO-d₆) δ: 9.38 (br s, 1H), 8.40 (br s, 1H), 7.79 (s, 2H), 7.57 (s, 1H), 7.00 (d, 1H), 6.84 (d, 1H), 4.19 (s, 3H), 1.78 (s, 3H), 1.71 (s, 3H).

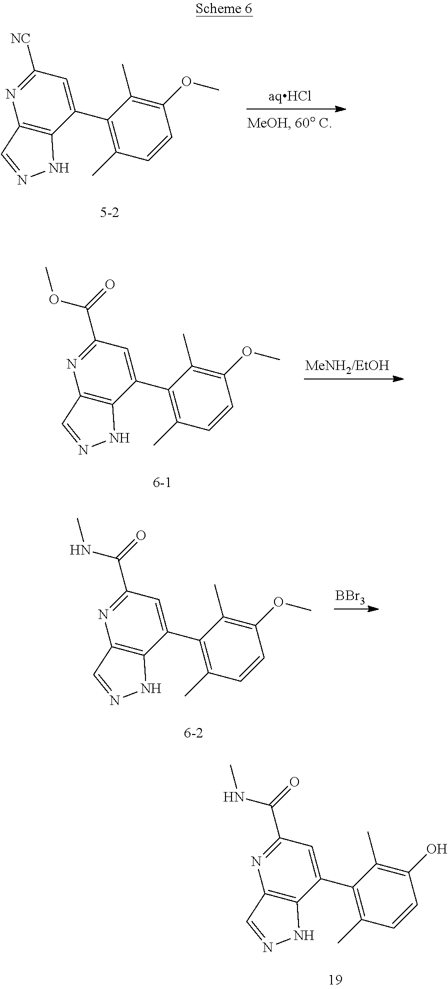

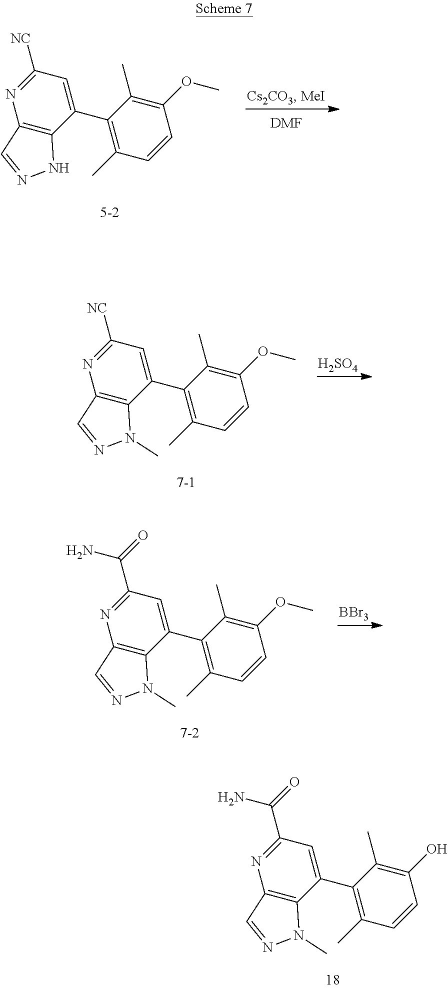

### Example 15. 7-(3-Hydroxy-2,6-dimethylphenyl)-1-methyl-1H-pyrazolo[4,3-b]pyridine-5-carboxamide (18)

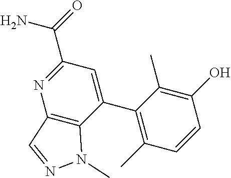 [SMILES: Cc1ccc(O)c(C)c1-c1cc(C(N)=O)nc2cnn(C)c12]

### Step 1: Synthesis of 7-(3-methoxy-2,6-dimethylphenyl)-1-methyl-pyrazolo[4,3-b]pyridine-5-carbonitrile

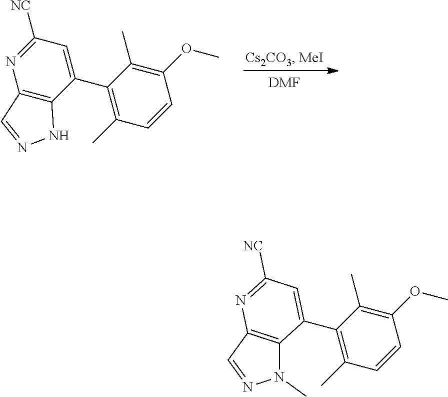

To a solution of 7-(3-methoxy-2,6-dimethylphenyl)-1H-pyrazolo[4,3-b]pyridine-5-carbonitrile (150 mg, 0.54 mmol, 1 eq) and Cs₂CO₃ (351 mg, 1.08 mmol, 2 eq) in DMF (1.5 mL) was added MeI (0.1 mL, 0.70 mmol, 1.3 eq) at 0° C. The mixture was stirred at 25° C. for 12 hours. The reaction mixture was quenched by the addition of water (10 mL), extracted with EtOAc (10 mL×3). The combined organic layers were washed with aq. saturated NaCl (10 mL×3), dried over anhyd. Na₂SO₄, filtered and concentrated. The residue was purified by column chromatography on silica gel (0%-8% Ethyl acetate in Petroleum ether) to give 7-(3-methoxy-2,6-dimethylphenyl)-1-methyl-pyrazolo[4,3-b]pyridine-5-carbonitrile (116 mg, 0.38 mmol, 71% yield, 96% purity). LC-MS m/z 293 (M+H⁺).

### Step 2: Synthesis of 7-(3-methoxy-2,6-dimethylphenyl)-1-methyl-pyrazolo[4,3-b]pyridine-5-carboxamide

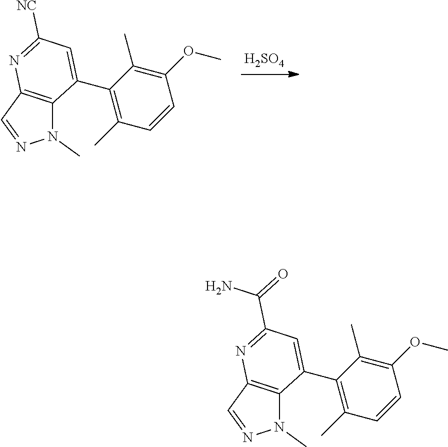

A mixture of 7-(3-methoxy-2,6-dimethylphenyl)-1-methyl-pyrazolo[4,3-b]pyridine-5-carbonitrile (106 mg, 0.36 mmol, 1 eq) in H₂SO₄ (1 mL) was stirred at 25° C. for 1 hour. The reaction mixture was adjusted pH=7 by aq. NH₃·H₂O, filtered to give the filter cake, which was concentrated to give 7-(3-methoxy-2,6-dimethylphenyl)-1-methyl-pyrazolo[4,3-b]pyridine-5-carboxamide (80 mg, 0.24 mmol, 67% yield, 94% purity). LC-MS m/z 311 (M+H⁺).

### Step 3: Synthesis of 7-(3-hydroxy-2,6-dimethylphenyl)-1-methyl-pyrazolo[4,3-b]pyridine-5-carboxamide (compound 18)

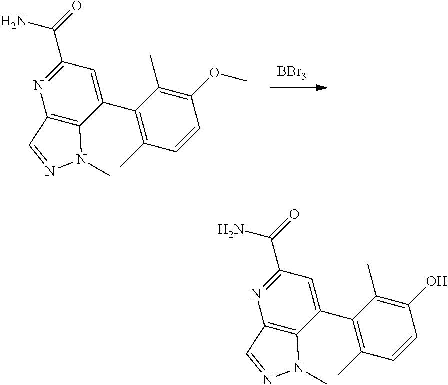

To a solution of 7-(3-methoxy-2,6-dimethylphenyl)-1-methyl-pyrazolo[4,3-b]pyridine-5-carboxamide (70 mg, 0.23 mmol, 1 eq) in DCM (2 mL) was added BBr₃ (0.1 mL, 1.13 mmol, 5 eq) at 0° C. The mixture was stirred at 25° C. for 1 hour. The reaction mixture was added dropwise into water (10 mL) at 25° C. The reaction mixture was filtered to give the filter cake, which was dried to give the crude product. The material was purified by RP-HPLC (column: Welch Xtimate C18 150×30 mm×5 um; mobile phase: [water(FA)-ACN]; B %: 4%-44%, 30 min) to afford 7-(3-hydroxy-2,6-dimethylphenyl)-1-methyl-pyrazolo[4,3-b]pyridine-5-carboxamide (29.4 mg, 0.10 mmol, 43% yield, 97% purity). LC-MS m/z 297 (M+H⁺). ¹H NMR (400 MHz, DMSO-d₆) δ=9.48 (s, 1H), 8.46 (s, 1H), 8.21 (s, 1H), 7.73-7.63 (m, 2H), 7.05 (d, 1H), 6.90 (d, 1H), 3.47 (s, 3H), 1.78 (s, 3H), 1.72 (s, 3H).

### Example 16. 7-(3-Hydroxy-2,6-dimethylphenyl)-N-methyl-1H-pyrazolo[4,3-b]pyridine-5-carboxamide (19)

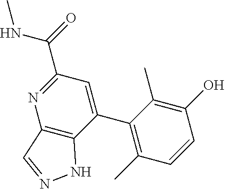 [SMILES: CNC(=O)c1cc(-c2c(C)ccc(O)c2C)c2[nH]ncc2n1]

### Step 1: Synthesis of methyl 7-(3-methoxy-2,6-dimethylphenyl)-1H-pyrazolo[4,3-b]pyridine-5-carboxylate

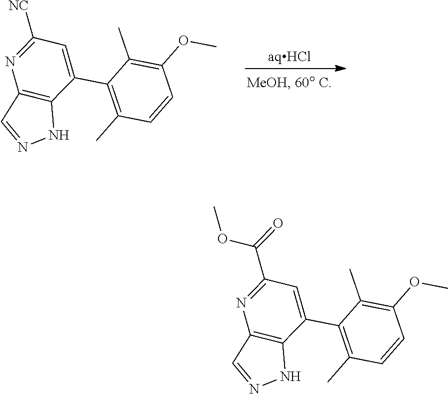

To a solution of 7-(3-methoxy-2,6-dimethylphenyl)-1H-pyrazolo[4,3-b]pyridine-5-carbonitrile (200 mg, 0.72 mmol, 1 eq) in MeOH (3 mL) was added aq. HCl (3 mL, 12 M). The mixture was stirred at 65° C. for 12 hours. The reaction was diluted with H₂O (20 mL), filtered to give the filter cake, which was concentrated to give methyl 7-(3-methoxy-2,6-dimethylphenyl)-1H-pyrazolo[4,3-b]pyridine-5-carboxylate (156 mg, 0.30 mmol, 60% purity). LC-MS m/z 312 (M+H⁺).

### Step 2: Synthesis of 7-(3-methoxy-2,6-dimethylphenyl)-N-methyl-1H-pyrazolo[4,3-b]pyridine-5-carboxamide

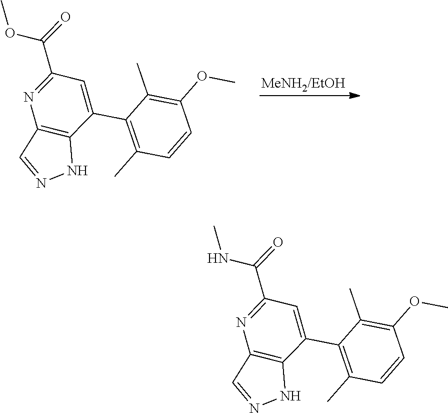

To a solution of methyl 7-(3-methoxy-2,6-dimethylphenyl)-1H-pyrazolo[4,3-b]pyridine-5-carboxylate (106 mg, 0.20 mmol, 60% purity, 1 eq) in EtOH (2 mL) was added MeNH₂ (2.21 g, 21.35 mmol, 30% in EtOH). The mixture was stirred at 30° C. for 2.5 hours. The reaction mixture was filtered to give the filter cake, which was concentrated to give 7-(3-methoxy-2,6-dimethylphenyl)-N-methyl-1H-pyrazolo[4,3-b]pyridine-5-carboxamide (115 mg, 0.30 mmol, 80% purity). LC-MS m/z 311 (M+H⁺).

### Step 3: Synthesis of 7-(3-hydroxy-2,6-dimethylphenyl)-N-methyl-1H-pyrazolo[4,3-b]pyridine-5-carboxamide (compound 19)

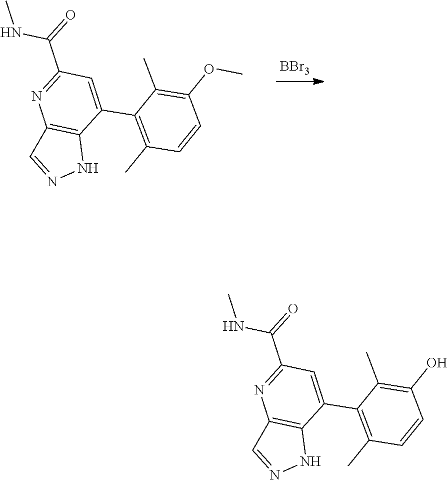

To a solution of 7-(3-methoxy-2,6-dimethylphenyl)-N-methyl-1H-pyrazolo[4,3-b]pyridine-5-carboxamide (95 mg, 0.31 mmol, 1 eq) in DCM (2 mL) was added BBr₃ (0.15 mL, 1.53 mmol, 5 eq) at 0° C. The mixture was stirred at 25° C. for 1 hour. The reaction mixture was added dropwise into water (10 mL) at 25° C., then filtered directly to give the crude product. The crude product was purified by RP-HPLC (column: Welch Xtimate C18 150×30 mm×5 um; mobile phase: [water (FA)-ACN]; B %: 2%-42%, 25 min) to give 7-(3-hydroxy-2,6-dimethylphenyl)-N-methyl-1H-pyrazolo[4,3-b]pyridine-5-carboxamide (47.5 mg, 0.16 mmol, 51% yield, 97% purity). LC-MS m/z 297 (M+H⁺). ¹H NMR (400 MHz, DMSO-d₆) δ=13.45 (s, 1H), 9.38 (s, 1H), 8.88-8.81 (m, 1H), 8.45 (s, 1H), 7.73 (s, 1H), 7.04 (d, 1H), 6.88 (d, 1H), 2.86 (d, 3H), 1.80 (s, 3H), 1.73 (s, 3H).

### Example 17. 7-(3-Hydroxy-2,6-dimethylphenyl)-N,N-dimethyl-1H-pyrazolo[4,3-b]pyridine-5-carboxamide (20)

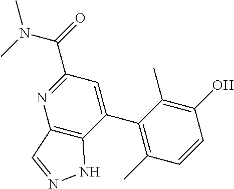 [SMILES: Cc1ccc(O)c(C)c1-c1cc(C(=O)N(C)C)nc2cn[nH]c12]

Compound 20 was synthesized according to Example 16, step 2 using dimethylamine instead of methylamine. LC-MS m/z 311 (M+H⁺). ¹H NMR (400 MHz, DMSO-d₆) δ=13.34 (m, 1H), 9.37 (s, 1H), 8.38 (s, 1H), 7.23 (s, 1H), 7.02 (d, 1H), 6.86 (d, 1H), 3.04 (d, 6H), 1.82 (s, 3H), 1.75 (s, 3H).

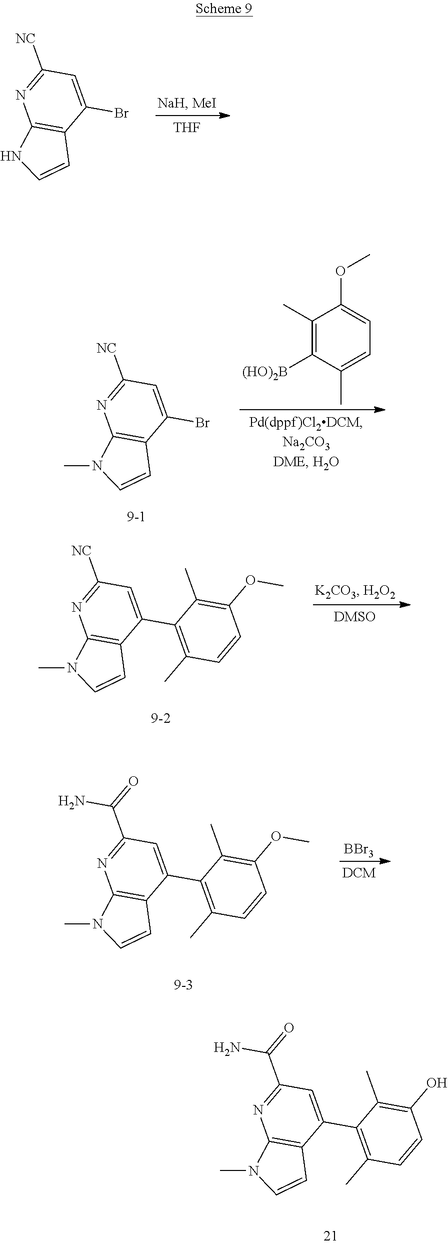

### Example 18. 4-(3-Hydroxy-2,6-dimethylphenyl)-1-methyl-1H-pyrrolo[2,3-b]pyridine-6-carboxamide (21)

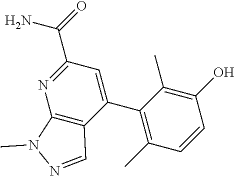 [SMILES: Cc1ccc(O)c(C)c1-c1cc(C(N)=O)nc2c1cnn2C]

### Step 1: Synthesis of 4-bromo-1-methyl-pyrrolo[2,3-b]pyridine-6-carbonitrile

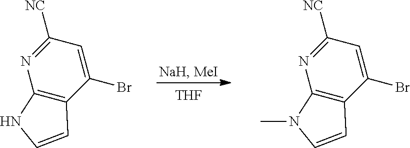

To a solution of 4-bromo-1H-pyrrolo[2,3-b]pyridine-6-carbonitrile (500 mg, 2.25 mmol, 1 eq) in THF (6 mL) was added NaH (180 mg, 4.50 mmol, 60% purity, 2 eq) at 0° C. After stirring at 0° C. for 0.5 hour, MeI (0.2 mL) was added. The mixture was stirred at 25° C. for another 16 hours. The reaction mixture was quenched by water (50 mL) and extracted with ethyl acetate (40 mL×3). The combined organic layers were washed with aq. saturated NaCl (30 mL×3), dried over anhyd. Na₂SO₄, filtered, and concentrated. The residue was purified by column chromatography on silica gel (0%-10% ethyl acetate in petroleum ether) to afford 4-bromo-1-methyl-pyrrolo[2,3-b]pyridine-6-carbonitrile (300 mg, 53% yield, 94% purity). LC-MS m/z 238 (M+H⁺).

### Step 2: Synthesis of 4-(3-methoxy-2,6-dimethylphenyl)-1-methyl-pyrrolo[2,3-b]pyridine-6-carbonitrile

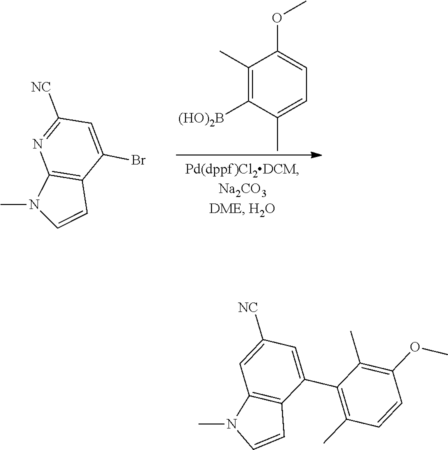

To a solution of 4-bromo-1-methyl-pyrrolo[2,3-b]pyridine-6-carbonitrile (300 mg, 1.27 mmol, 1 eq) and (3-methoxy-2,6-dimethylphenyl)boronic acid (343 mg, 1.91 mmol, 1.5 eq) in DME (6 mL) and H₂O (2 mL) was added Pd(dppf)Cl₂·CH₂Cl₂ (104 mg, 0.127 mmol, 0.1 eq) and Na₂CO₃ (1.35 g, 12.71 mmol, 10 eq). The mixture was irradiated at 120° C. for 3 hours in the microwave. The reaction mixture was diluted with water (50 mL) and extracted with ethyl acetate (40 mL×3). The combined organic layers were washed with aq. saturated NaCl (30 mL×3), dried over anhyd. Na₂SO₄, filtered and concentrated. The residue was purified by column chromatography on silica gel (0%-10% ethyl acetate in petroleum ether) to give 4-(3-methoxy-2,6-dimethylphenyl)-1-methyl-pyrrolo[2,3-b]pyridine-6-carbonitrile (290 mg, 71% yield, 91% purity). LC-MS m/z 292 (M+H⁺).

### Step 3: Synthesis of 4-(3-methoxy-2,6-dimethylphenyl)-1-methyl-pyrrolo[2,3-b]pyridine-6-carboxamide

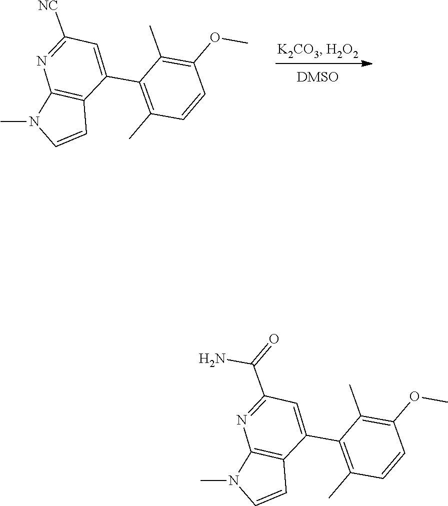

To a solution of 4-(3-methoxy-2,6-dimethylphenyl)-1-methyl-pyrrolo[2,3-b]pyridine-6-carbonitrile (290 mg, 0.995 mmol, 1 eq) and K₂CO₃ (206 mg, 1.49 mmol, 1.5 eq) in DMSO (2 mL) was added H₂O₂ (4.2 mL, 30% purity) at 0° C. The mixture was stirred at 20° C. for 1 hour. The reaction mixture was diluted with water (40 mL), extracted with DCM (30 mL×3). The combined organic layers were washed with aq. saturated NaCl (20 mL×3), dried over anhyd. Na₂SO₄, filtered and concentrated. The residue was purified by column chromatography on silica gel (0%-50% ethyl acetate in petroleum ether) to afford 4-(3-methoxy-2,6-dimethylphenyl)-1-methyl-pyrrolo[2,3-b]pyridine-6-carboxamide (260 mg, 76% yield, 90% purity). LC-MS m/z 310 (M+H⁺).

### Step 4: Synthesis of 4-(3-hydroxy-2,6-dimethylphenyl)-1-methyl-pyrrolo[2,3-b]pyridine-6-carboxamide (compound 21)

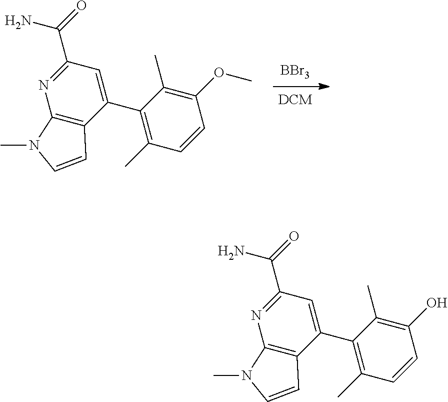

To a solution of 4-(3-methoxy-2,6-dimethylphenyl)-1-methyl-pyrrolo[2,3-b]pyridine-6-carboxamide (200 mg, 0.646 mmol, 1 eq) in DCM (2 mL) was added BBr₃ (0.2 mL, 3 eq). The mixture was stirred at 20° C. for 1 hour. The mixture was quenched with water (15 mL) and adjusted to pH 7 with aq. saturated NaHCO₃, then extracted with DCM (40 mL×3). The combined organic layers were washed with aq. saturated NaCl (20 mL×3), dried over anhyd. Na₂SO₄, filtered and concentrated. The residue was purified by column chromatography on silica gel (0%-50% ethyl acetate in petroleum ether), further purified by RP-HPLC ([water (NH₃H₂O+NH₄HCO₃)-ACN]; B %: 14%-54%, 30 min) to afford 4-(3-hydroxy-2,6-dimethylphenyl)-1-methyl-pyrrolo[2,3-b]pyridine-6-carboxamide (47.5 mg, 25% yield, 100% purity). LC-MS m/z 296 (M+H⁺). ¹H NMR (400 MHz, DMSO-d₆) δ=9.27 (s, 1H), 8.21 (s, 1H), 7.71-7.62 (d, 1H), 7.57 (s, 1H), 7.49 (s, 1H), 7.02-6.91 (d, 1H), 6.84-6.76 (d, 1H), 6.02 (d, 1H), 3.95 (s, 3H), 1.76 (s, 3H), 1.69 (s, 3H).

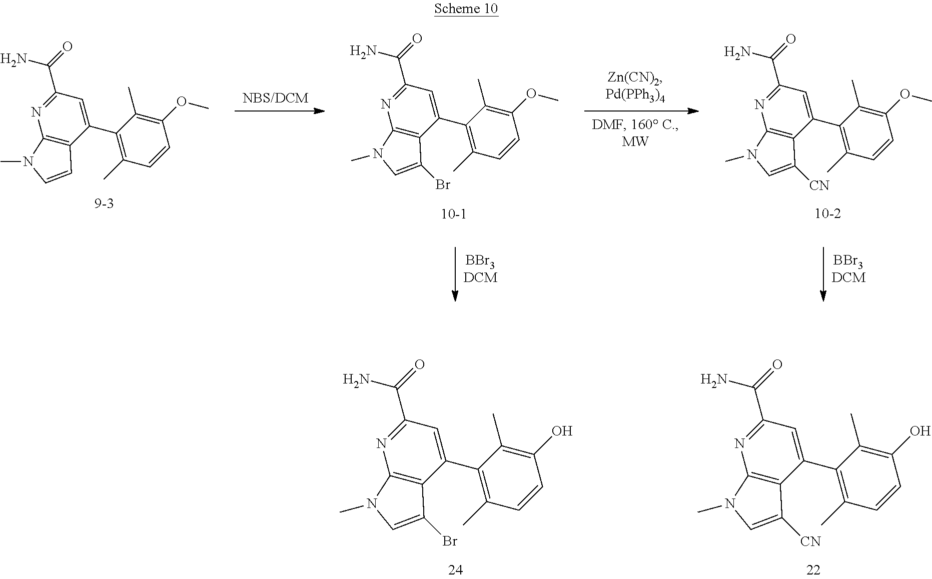

### Example 19. 3-Cyano-4-(3-hydroxy-2,6-dimethylphenyl)-1-methyl-1H-pyrrolo[2,3-b]pyridine-6-carboxamide (22)

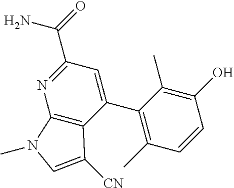 [SMILES: Cc1ccc(O)c(C)c1-c1cc(C(N)=O)nc2c1c(C#N)cn2C]

### Step 1: Synthesis of 3-bromo-4-(3-methoxy-2,6-dimethylphenyl)-1-methyl-pyrrolo[2,3-b]pyridine-6-carboxamide

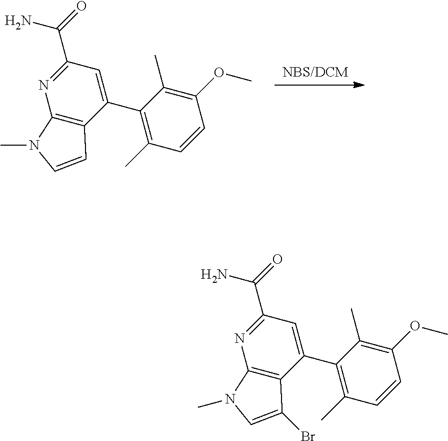

To a solution of 4-(3-methoxy-2,6-dimethylphenyl)-1-methyl-pyrrolo[2,3-b]pyridine-6-carboxamide (130 mg, 0.420 mmol, 1 eq) in CH₂Cl₂ (1 mL) was added NBS (52 mg, 0.294 mmol, 0.7 eq). The mixture was stirred at 25° C. for 3 hours. The mixture was diluted with water (40 mL) and extracted with DCM (30 mL×3). The combined organic layers were washed with aq. saturated NaCl (20 mL×3), dried over anhyd. Na₂SO₄, filtered and concentrated. The residue was purified by column chromatography on silica gel (30%-50% ethyl acetate in petroleum ether) to give 3-bromo-4-(3-methoxy-2,6-dimethylphenyl)-1-methyl-pyrrolo[2,3-b]pyridine-6-carboxamide (250 mg, crude). LC-MS m/z 390 (M+H⁺).

### Step 2: Synthesis of 3-cyano-4-(3-methoxy-2,6-dimethylphenyl)-1-methyl-pyrrolo[2,3-b]pyridine-6-carboxamide

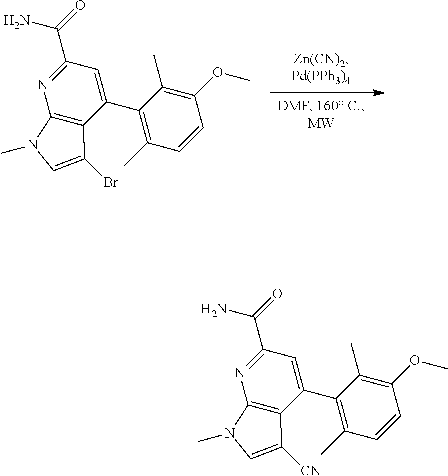

To a solution of 3-bromo-4-(3-methoxy-2,6-dimethylphenyl)-1-methyl-pyrrolo[2,3-b]pyridine-6-carboxamide (200 mg, 0.52 mmol, 1 eq) in DMF (4 mL) was added Zn(CN)₂ (660 mg, 5.62 mmol, 10 eq) and Pd(PPh₃)₄ (119 mg, 0.1 mol, 0.2 eq). The mixture was degassed with N₂ and stirred at 160° C. for 3 hours under microwave irradiation. The reaction mixture was diluted with water (40 mL), extracted with ethyl acetate (30 mL×3). The combined organic layers were dried over anhyd. Na₂SO₄, filtered and concentrated. The residue was purified by column chromatography on silica gel (0%-30% ethyl acetate in petroleum ether) to give 3-cyano-4-(3-methoxy-2,6-dimethylphenyl)-1-methyl-pyrrolo[2,3-b]pyridine-6-carboxamide (170 mg, crude). LC-MS m/z 335 (M+H⁺).

### Step 3: Synthesis of 3-cyano-4-(3-hydroxy-2,6-dimethylphenyl)-1-methyl-pyrrolo[2,3-b]pyridine-6-carboxamide (compound 22)

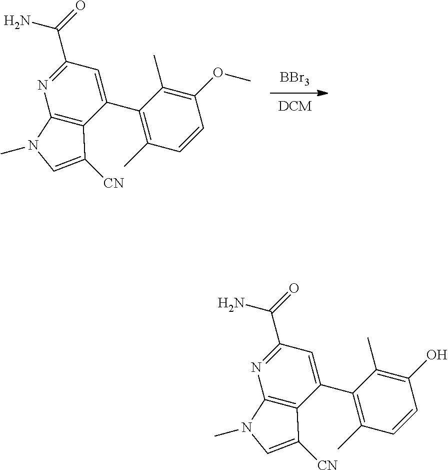

To a solution of 3-cyano-4-(3-methoxy-2,6-dimethylphenyl)-1-methyl-pyrrolo[2,3-b]pyridine-6-carboxamide (150 mg, 0.45 mmol, 1 eq) in DCM (3 mL) was added BBr₃ (0.1 mL, 3 eq) at 0° C. under N₂. The mixture was stirred at 20° C. for 1 hour. The reaction mixture was quenched by water (20 mL) at 25° C., extracted with DCM (30 mL×3). The combined organic layers were washed with aq. Saturated NaCl (20 mL×3), dried over anhyd. Na₂SO₄, filtered and concentrated. The crude product was purified by prep-HPLC([water(NH₃H₂O+NH₄HCO₃)-ACN]; B %: 6%-46%, 36 min) to give 3-cyano-4-(3-hydroxy-2,6-dimethylphenyl)-1-methyl-pyrrolo[2,3-b]pyridine-6-carboxamide (15.8 mg, 0.049 mmol, 11% yield, 99% purity). LC-MS m/z 321 (M+H⁺). ¹H NMR (400 MHz, DMSO-d₆) δ=9.27 (s, 1H), 8.59 (s, 1H), 8.35 (s, 1H), 7.74 (s, 1H), 7.64 (s, 1H), 6.96 (d, 1H), 6.81 (d, 1H), 4.01 (s, 3H), 1.76 (s, 3H), 1.69 (s, 3H).

### Example 20. 3-Cyano-4-(3-hydroxy-2,6-dimethylphenyl)-1-(2,2,2-trifluoroethyl)-1H-pyrrolo[2,3-b]pyridine-6-carboxamide (23)

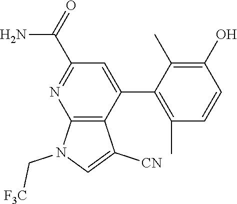 [SMILES: Cc1ccc(O)c(C)c1-c1cc(C(N)=O)nc2c1c(C#N)cn2CC(F)(F)F]

### Step 1: Synthesis of 4-bromo-1-(2, 2, 2-trifluoroethyl) pyrrolo[2,3-b]pyridine-6-carbonitrile

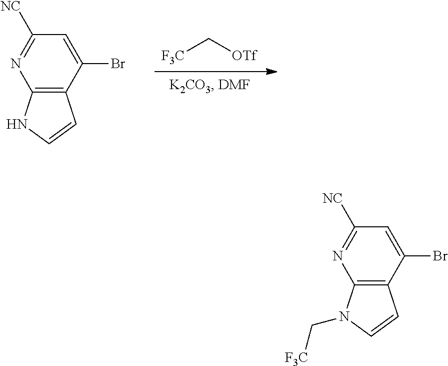

To a solution of 4-bromo-1H-pyrrolo[2,3-b]pyridine-6-carbonitrile (1 g, 4.50 mmol, 1 eq) in DMF (10 mL) was added K₂CO₃ (1.87 g, 13.5 mmol, 3 eq) and 2,2,2-trifluoroethyl trifluoromethane sulfonate (1.57 g, 6.76 mmol, 1.5 eq). The mixture was stirred at 80° C. for 5 hours. The mixture was diluted with water (30 mL) and extracted with DCM (30 mL×3). The combined organic layers were washed with aq. saturated NaCl (20 mL×3), dried over anhyd. Na₂SO₄, filtered and concentrated. The residue was purified by column chromatography on silica gel (0%-20% ethyl acetate in petroleum ether) to give 4-bromo-1-(2, 2, 2-trifluoroethyl) pyrrolo[2,3-b]pyridine-6-carbonitrile (900 mg, crude). LC-MS m/z 304 (M+H⁺).

### Step 2: Synthesis of 4-(3-methoxy-2,6-dimethylphenyl)-1-(2,2,2-trifluoroethyl) pyrrolo[2,3-b]pyridine-6-carbonitrile

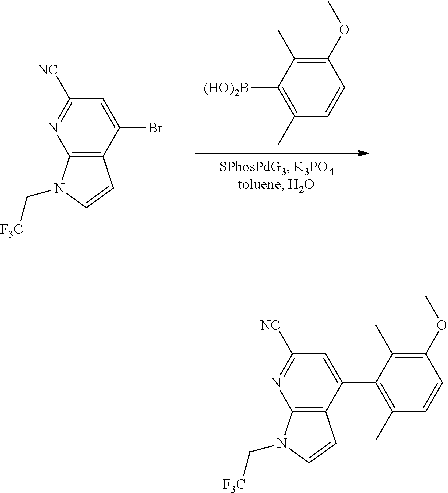

To a solution of 4-bromo-1-(2,2,2-trifluoroethyl) pyrrolo[2,3-b]pyridine-6-carbonitrile (900 mg, crude) in H₂O (2 mL) and toluene (10 mL) were added K₃PO₄ (2.51 g, 11.8 mmol, 4 eq), (3-methoxy-2,6-dimethylphenyl) boronic acid (1.07 g, 5.92 mmol, 2 eq) and SPhos Pd G3 (230 mg, 0.3 mmol, 0.1 eq). The mixture was stirred at 80° C. for 16 hours under N₂. The reaction mixture was diluted with water (40 mL), extracted with DCM (10 mL×3). The combined organic layers were washed with aq. saturated NaCl (10 mL×3), dried over anhyd. Na₂SO₄, filtered and concentrated. The residue was purified by column chromatography on silica gel (0%-20% Ethyl acetate in Petroleum ether) to give 4-(3-methoxy-2,6-dimethylphenyl)-1-(2,2,2-trifluoroethyl) pyrrolo[2,3-b]pyridine-6-carbonitrile (950 mg, crude). LC-MS m/z 360 (M+H⁺).

### Step 3: Synthesis of 4-(3-methoxy-2, 6-dimethylphenyl)-1-(2, 2, 2-trifluoroethyl)pyrrolo[2,3-b]pyridine-6-carboxamide

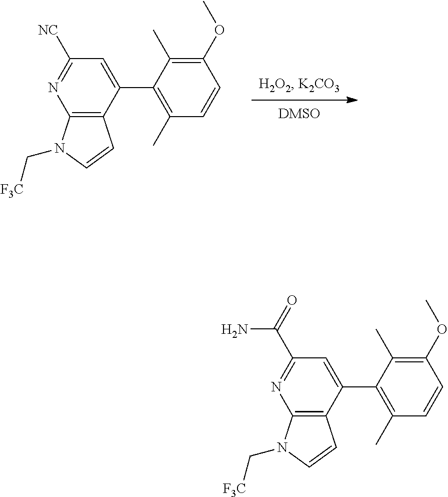

To a solution of 4-(3-methoxy-2,6-dimethylphenyl)-1-(2,2,2-trifluoroethyl)pyrrolo[2,3-b]pyridine-6-carbonitrile (1.96 g, 5.45 mmol, 1 eq) in DMSO (40 mL) was added K₂CO₃ (1.13 g, 8.18 mmol, 1.5 eq), H₂O₂ (12 mL, 30% purity) at 0° C. The mixture was stirred at 25° C. for 0.5 hour. The reaction mixture was diluted with H₂O (40 mL). The mixture was filtered and concentrated directly to give compound 4-(3-methoxy-2, 6-dimethylphenyl)-1-(2, 2, 2-trifluoroethyl)pyrrolo[2,3-b]pyridine-6-carboxamide (1.6 g, 80% yield). LC-MS m/z 378 (M+H⁺).

### Step 4: Synthesis of 3-bromo-4-(3-methoxy-2,6-dimethylphenyl)-1-(2,2,2-trifluoroethyl)pyrrolo[2,3-b]pyridine-6-carboxamide

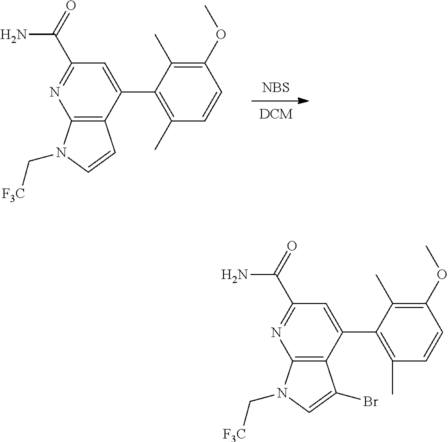

To a solution of 4-(3-methoxy-2,6-dimethylphenyl)-1-(2,2,2-trifluoroethyl)pyrrolo[2,3-b]pyridine-6-carboxamide (1.6 g, 4.24 mmol, 1 eq) in DCM (30 mL) was added NBS (754 mg, 4.24 mmol, 1 eq). The mixture was stirred at 25° C. for 16 hours. The reaction mixture was concentrated to give a residue, which was purified by column chromatography on silica gel (30%-50% ethyl acetate in petroleum ether) to give compound 3-bromo-4-(3-methoxy-2,6-dimethylphenyl)-1-(2,2,2-trifluoroethyl)pyrrolo[2,3-b]pyridine-6-carboxamide (1.2 g, 62% yield). LC-MS m/z 458 (M+H⁺).

### Step 5: Synthesis of 3-cyano-4-(3-methoxy-2,6-dimethylphenyl)-1-(2,2,2-trifluoroethyl)pyrrolo[2,3-b]pyridine-6-carboxamide

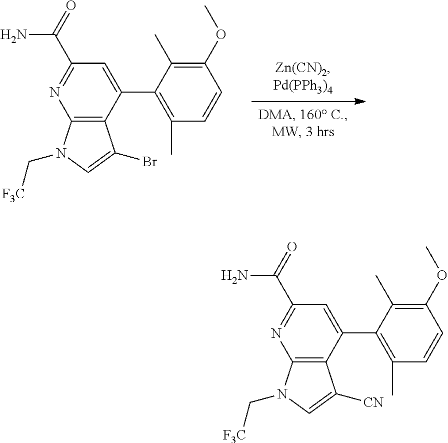

To a solution of 3-bromo-4-(3-methoxy-2,6-dimethylphenyl)-1-(2,2,2-trifluoroethyl)pyrrolo[2,3-b]pyridine-6-carboxamide (1.19 g, 2.61 mmol, 1 eq) in DMF (10 mL) was added Zn(CN)₂ (3.06 g, 26.1 mmol, 1.66 mL, 10 eq) and Pd(PPh₃)₄ (602 mg, 0.52 mmol, 0.2 eq) under N₂ atmosphere. The mixture was stirred at 160° C. for 3 hours using microwave. The reaction mixture was diluted with water (40 mL), extracted with ethyl acetate (30 mL×3). The combined organic layers were washed with aq. NaCl (20 mL×3), dried over anhyd. Na₂SO₄, filtered and concentrated. The residue was purified by column chromatography on silica gel (0%-30% ethyl acetate in petroleum ether) to afford compound 3-cyano-4-(3-methoxy-2,6-dimethylphenyl)-1-(2,2,2-trifluoroethyl)pyrrolo[2,3-b]pyridine-6-carboxamide (650 mg, 57% yield, 92% purity). LC-MS m/z 403 (M+H⁺).

### Step 6: Synthesis of 3-cyano-4-(3-hydroxy-2,6-dimethylphenyl)-1-(2,2,2-trifluoroethyl) pyrrolo[2,3-b]pyridine-6-carboxamide (compound 23)

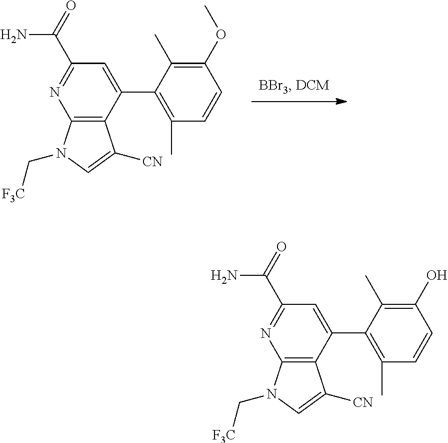

To a solution of 3-cyano-4-(3-methoxy-2,6-dimethylphenyl)-1-(2,2,2-trifluoroethyl)pyrrolo[2,3-b]pyridine-6-carboxamide (170 mg, 0.42 mmol, 1 eq) in DCM (5 mL) was added BBr₃ (0.2 mL) at 0° C. The mixture was stirred at 25° C. for 1 hour. The reaction mixture quenched with water (1 mL), filtered to give the filtration. The mixture was concentrated to give a residue, which was purified by RP-HPLC (column: Welch Xtimate C18 150×30 mm×5 um; mobile phase: [water(NH₃H₂O+NH₄HCO₃)-ACN]; B %: 12%-52%, 28 min) to give compound 3-cyano-4-(3-hydroxy-2,6-dimethylphenyl)-1-(2,2,2-trifluoroethyl)pyrrolo[2,3-b]pyridine-6-carboxamide (97 mg, 0.25 mmol, 59% yield, 100% purity). LC-MS m/z 389 (M+H⁺). ¹H NMR (400 MHz, DMSO-d₆) δ=9.30 (s, 1H), 8.73 (s, 1H), 8.63 (d, 1H), 7.78 (d, 1H), 7.69 (s, 1H), 6.98 (d, 1H), 6.82 (d, 1H), 5.54 (q, 2H), 1.77 (s, 3H), 1.70 (s, 3H). ¹⁹F NMR: −69.975

### Example 21. 3-Bromo-4-(3-hydroxy-2,6-dimethylphenyl)-1-methyl-1H-pyrrolo[2,3-b]pyridine-6-carboxamide (24)

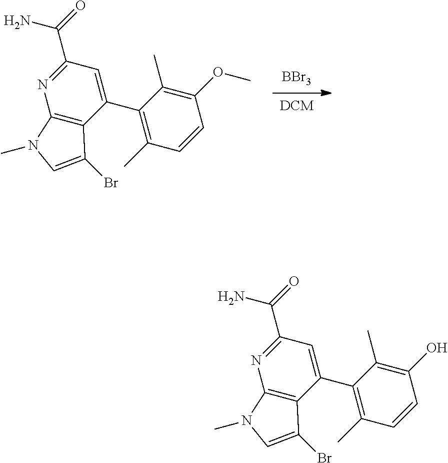

To a solution of 3-bromo-4-(3-methoxy-2,6-dimethylphenyl)-1-methyl-pyrrolo[2,3-b]pyridine-6-carboxamide (380 mg, 0.98 mmol, 1 eq) in DCM (2 mL) was added BBr₃ (0.3 mL, 3 eq) at 0° C. The mixture was stirred at 20° C. for 16 hours. The reaction mixture was quenched by water (5 mL), diluted with water (30 mL), and extracted with DCM (20 mL×3). The combined organic layers were washed with aq. saturated. NaCl (10 mL×2), dried over anhyd. Na₂SO₄, filtered and concentrated. The residue was purified by prep-HPLC (column: Welch Xtimate C18 150×30 mm×5 um; mobile phase: [water(NH₃H₂O+NH₄HCO₃)-ACN]; B %: 14%-54%, 28 min) to give 3-bromo-4-(3-hydroxy-2,6-dimethylphenyl)-1-methyl-pyrrolo[2,3-b]pyridine-6-carboxamide (14.4 mg). LC-MS m/z 376 (M+H⁺). ¹H NMR (400 MHz, DMSO-d6) δ=9.18 (s, 1H), 8.25 (d, 1H), 7.88 (s, 1H), 7.63 (d, 1H), 7.46 (s, 1H), 6.92 (d, 1H), 6.78 (d, 1H), 3.94 (s, 3H), 1.73 (s, 3H), 1.66 (s, 3H).

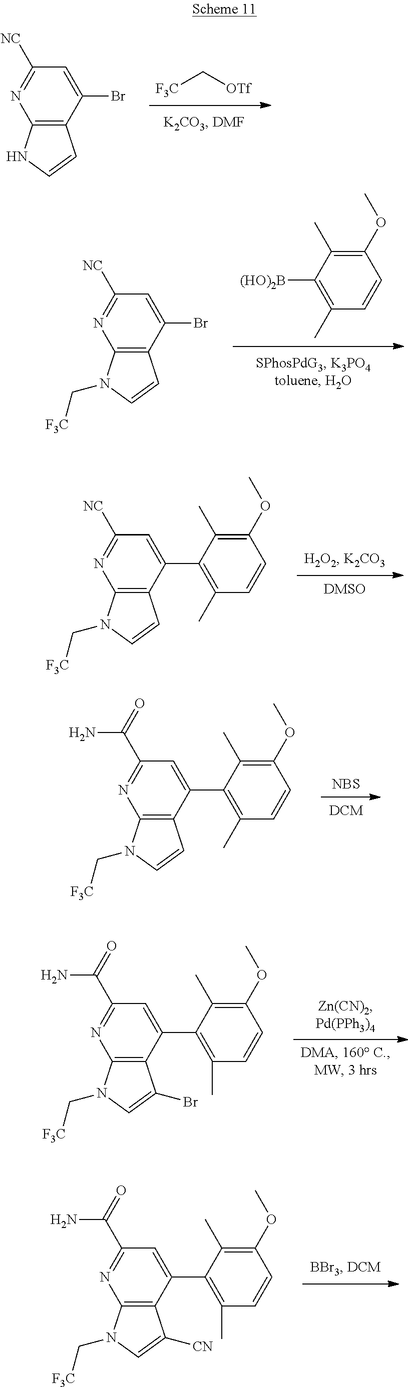

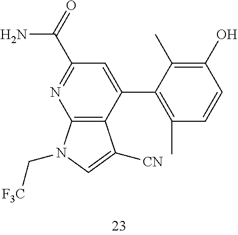 [SMILES: Cc1ccc(O)c(C)c1-c1cc(C(N)=O)nc2c1c(C#N)cn2CC(F)(F)F]

### Example 22. (M)-3-Cyano-4-(3-hydroxy-2,6-dimethylphenyl)-1-(2,2,2-trifluoroethyl)-1H-pyrrolo[2,3-b]pyridine-6-carboxamide (25) and (P)-3-cyano-4-(3-hydroxy-2,6-dimethylphenyl)-1-(2,2,2-trifluoroethyl)-1H-pyrrolo[2,3-b]pyridine-6-carboxamide (27)

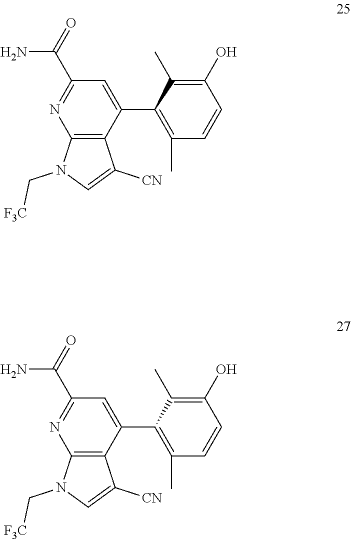

Compound 23 (87 mg, racemate) was resolved by SFC separation (column: DAICEL CHIRALPAK IG (250 mm×30 mm, 10 um); mobile phase: [0.1% NH₃H₂O ETOH]; B %: 30%-30%, min) to give (M)-3-cyano-4-(3-hydroxy-2,6-dimethylphenyl)-1-(2,2,2-trifluoroethyl)pyrrolo[2,3-b]pyridine-6-carboxamide (compound 25, 32 mg, 95.77% purity) and (P)-3-cyano-4-(3-hydroxy-2,6-dimethylphenyl)-1-(2,2,2-trifluoroethyl)pyrrolo[2,3-b]pyridine-6-carboxamide (compound 27, 32 mg, 98.37% purity).

Peak 1. SFC t_{R}=1.318 min in Column: Chiralpak IG 50×4.6 mm I.D., 4.6 um Mobile phase: A: CO₂ B: Ethanol (0.05% DEA) Gradient: from 5% to 40% of B in 2.5 min and hold 40% of 1 min, then 5% of B for 1 min Flow rate:4 mL/min Column temp.:35° C. ABPR: 1500 psi. LC-MS m/z 389 (M+H⁺). ¹H NMR (400 MHz, DMSO-d₆) δ=9.30 (s, 1H), 8.72 (s, 1H), 8.63 (d, 1H), 7.78 (d, 1H), 7.69 (s, 1H), 6.98 (d, 1H), 6.83 (d, 1H), 5.54 (m, 2H), 1.77 (s, 3H), 1.70 (s, 3H), ¹⁹F NMR: −69.23.

Peak 2. SFC t_{R}=1.652 min in Column: Chiralpak IG 50×4.6 mm I.D., 4.6 um Mobile phase: A: CO₂ B: Ethanol (0.05% DEA) Gradient: from 5% to 40% of B in 2.5 min and hold 40% of 1 min, then 5% of B for 1 min Flow rate:4 mL/mm Column temp.:35° C. ABPR: 1500 psi. LC-MS m/z 389 (M+H⁺). ¹H NMR (400 MHz, DMSO-d₆) δ=9.32 (s, 1H), 8.73 (s, 1H), 8.63 (d, 1H), 7.78 (d, 1H), 7.69 (s, 1H), 7.00 (d, 1H), 6.84 (d, 1H), 5.58 (m, 2H), 1.77 (s, 3H), 1.70 (s, 3H). ¹⁹F NMR: −69.22.

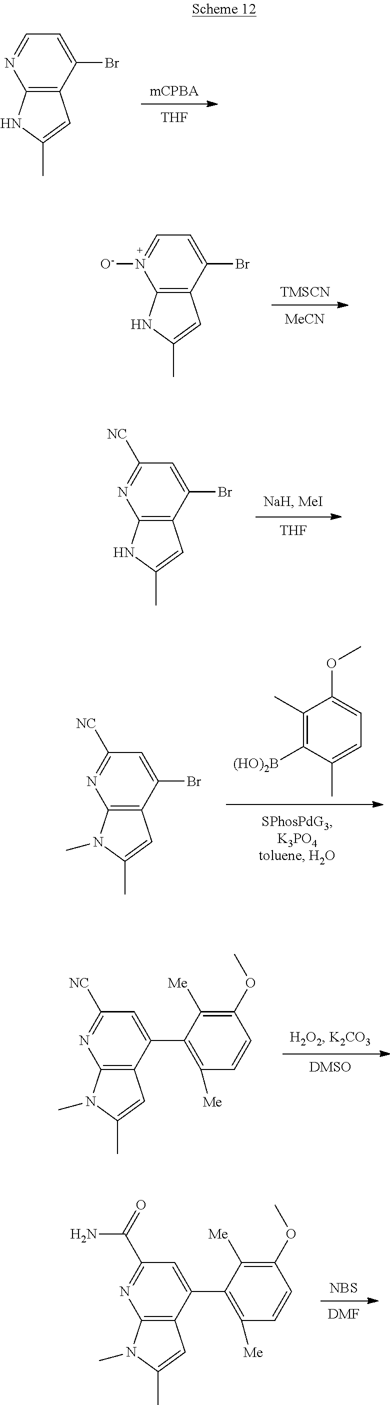

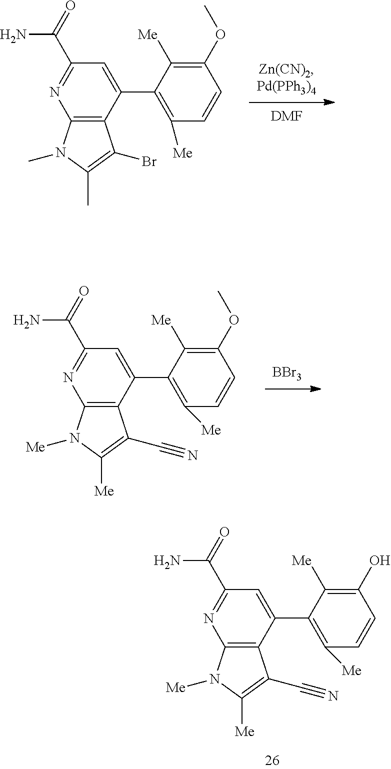

### Example 23. 3-Cyano-4-(3-hydroxy-2,6-dimethylphenyl)-1,2-dimethyl-1H-pyrrolo[2,3-b]pyridine-6-carboxamide (26)

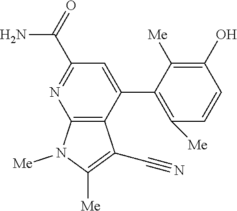 [SMILES: Cc1ccc(O)c(C)c1-c1cc(C(N)=O)nc2c1c(C#N)c(C)n2C]

### Step 1: Synthesis of 4-bromo-2-methyl-7-oxido-1H-pyrrolo[2,3-b]pyridin-7-ium

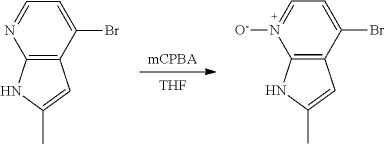

To a solution of 4-bromo-2-methyl-1H-pyrrolo[2,3-b]pyridine (4.5 g, 21.32 mmol, 1 eq) in THF (50 mL) was added m-CPBA (8.66 g, 42.64 mmol, 85% purity, 2 eq). The mixture was stirred at 50° C. for 2 hours. The reaction mixture was quenched by aq. saturated Na₂SO₃ (50 mL) at 0° C. and stirred at 25° C. for 16 hours. The mixture was filtered and collect the filter cake to give 4-bromo-2-methyl-7-oxido-1H-pyrrolo[2,3-b]pyridin-7-ium (1.48 g. crude) and was used directly into the next step. LC-MS m/z 229 (M+H⁺).

### Step 2: Synthesis of 4-bromo-2-methyl-1H-pyrrolo[2,3-b]pyridine-6-carbonitrile

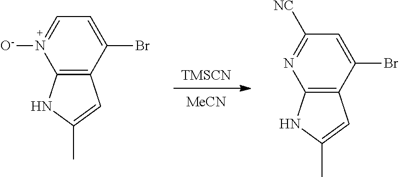

To a solution of 4-bromo-2-methyl-7-oxido-1H-pyrrolo[2,3-b]pyridin-7-ium (1.48 g, 6.52 mmol, 1 eq) in MeCN (20 mL) was added TMSCN (6.47 g, 65.18 mmol, 8.2 mL, 10 eq). After stirring at 80° C. for 70 hours, the mixture was concentrated under vacuum to give 4-bromo-2-methyl-1H-pyrrolo[2,3-b]pyridine-6-carbonitrile (1.52 g, crude). LC-MS m/z 236 (M+H⁺).

### Step 3: Synthesis of 4-bromo-1,2-dimethyl-pyrrolo[2,3-b]pyridine-6-carbonitrile

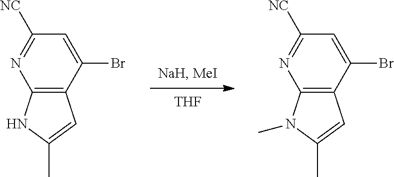

To a solution of 4-bromo-2-methyl-1H-pyrrolo[2,3-b]pyridine-6-carbonitrile (1.42 g, crude) in DMF (15 mL) was added K₂CO₃ (2.49 g, 18.05 mmol, 3 eq) and MeI (0.5 mL, 1.2 eq). The mixture was stirred at 25° C. for 16 hours. The reaction mixture was diluted with water (20 mL). The solids were collected by vacuum filtration to give 4-bromo-1,2-dimethyl-pyrrolo[2,3-b]pyridine-6-carbonitrile (1.23 g, crude). LC-MS m/z 250 (M+H⁺). ¹H NMR: (400 MHz, DMSO-d₆) δ=7.95 (s, 1H), 6.42 (s, 1H), 3.75 (s, 3H), 2.52 (s, 3H).

### Step 4: Synthesis of 4-(3-methoxy-2,6-dimethylphenyl)-1,2-dimethyl-pyrrolo[2,3-b]pyridine-6-carbonitrile

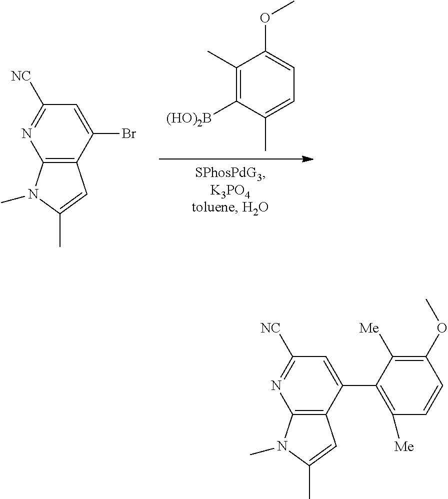

To a solution of 4-bromo-1,2-dimethyl-pyrrolo[2,3-b]pyridine-6-carbonitrile (1.23 g, 4.92 mmol, 1 eq) and (3-methoxy-2,6-dimethylphenyl)boronic acid (1.77 g, 9.84 mmol, 2 eq) in toluene (10 mL) and H₂O (2 mL) was added SPhos Pd G3 (384 mg, 0.49 mmol, 0.1 eq) and K₃PO₄ (3.13 g, 14.75 mmol, 3 eq). The mixture was stirred at 100° C. under N₂ for 16 hours. The reaction mixture was diluted with water (50 mL) and extracted with EtOAc (3×50 mL). The combined organic layers were washed with aq. saturated NaCl (2×50 mL), dried over anhyd. Na₂SO₄, filtered and concentrated to give a residue. The residue was purified by flash silica gel chromatography (ISCOC® 20 g SepaFlash® Silica Flash Column. Eluent of 0~4% Ethyl acetate/Petroleum ether gradient @45 mL/min) to give 4-(3-methoxy-2,6-dimethylphenyl)-1,2-dimethyl-pyrrolo[2,3-b]pyridine-6-carbonitrile (1.28 g, 83% yield, 98% purity). LC-MS m/z 306 (M+H⁺). ¹H NMR (400 MHz, DMSO-d₆) δ=7.42 (s, 1H), 7.15 (d, 1H), 6.97 (d, 1H), 5.93 (s, 1H), 3.80 (d, 6H), 2.46 (s, 3H), 1.82 (s, 3H), 1.73 (s, 3H).

### Step 5: Synthesis of 4-(3-methoxy-2,6-dimethylphenyl)-1,2-dimethyl-pyrrolo[2,3-b]pyridine-6-carboxamide

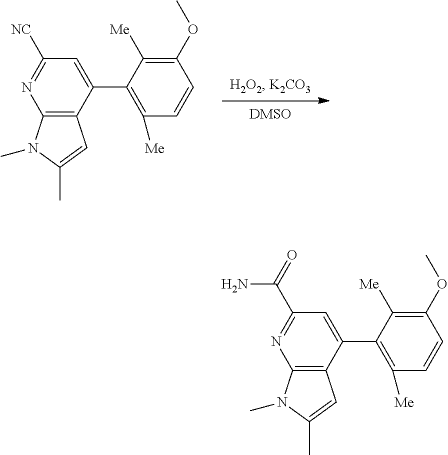

To a solution of 4-(3-methoxy-2,6-dimethylphenyl)-1,2-dimethyl-pyrrolo[2,3-b]pyridine-6-carbonitrile (1.28 g, 4.19 mmol, 1 eq) in DMSO (10 mL) was added K₂CO₃ (869 mg, 6.29 mmol, 1.5 eq) and H₂O₂ (4.6 mL, 30% purity) at 0° C. The mixture was stirred at 25° C. for 2 hours. The reaction mixture was diluted with water (20 mL). The mixture was filtered to give the filter cake, which was concentrated to give 4-(3-methoxy-2,6-dimethylphenyl)-1,2-dimethyl-pyrrolo[2,3-b]pyridine-6-carboxamide (1.1 g, 79% yield, 97% purity). LC-MS m/z 324 (M+H⁺).

### Step 6: Synthesis of 3-bromo-4-(3-methoxy-2,6-dimethylphenyl)-1,2-dimethyl-pyrrolo[2,3-b]pyridine-6-carboxamide

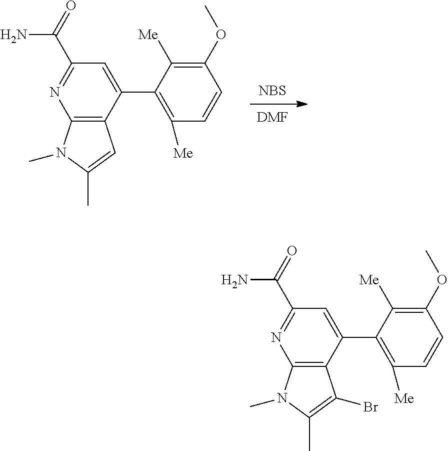

To a solution of 4-(3-methoxy-2,6-dimethylphenyl)-1,2-dimethyl-pyrrolo[2,3-b]pyridine-6-carboxamide (1.1 g, 3.40 mmol, 1 eq) in DMF (10 mL) was added NBS (605 mg, 3.40 mmol, 1 eq). The mixture was stirred at 25° C. for 16 hours. The reaction mixture was diluted with water (20 mL). The mixture was filtered directly to give 3-bromo-4-(3-methoxy-2,6-dimethylphenyl)-1,2-dimethyl-pyrrolo[2,3-b]pyridine-6-carboxamide (1.4 g, 96% yield, 94% purity). LC-MS m/z 404 (M+H⁺). ¹H NMR: (400 MHz, DMSO-d₆) δ=8.31 (s, 1H), 7.59 (s, 1H), 7.43 (s, 1H), 7.09 (d, 1H), 6.93 (d, 1H), 3.92 (s, 3H), 3.80 (s, 3H), 2.44 (s, 3H), 1.76 (s, 3H), 1.69 (s, 3H).

### Step 7: Synthesis of 3-cyano-4-(3-methoxy-2,6-dimethylphenyl)-1,2-dimethyl-pyrrolo[2,3-b]pyridine-6-carboxamide

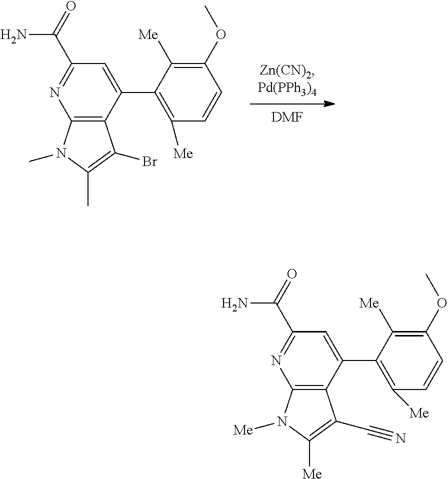

To a solution of 3-bromo-4-(3-methoxy-2,6-dimethylphenyl)-1,2-dimethyl-pyrrolo[2,3-b]pyridine-6-carboxamide (600 mg, 1.49 mmol, 1 eq) in DMF (5 mL) was added Zn(CN)₂ (1.65 g, 14.05 mmol, 9.42 eq) and Pd(PPh₃)₄ (345 mg, 0.30 mmol, 0.2 eq). The mixture was degassed with N₂ and stirred at 160° C. for 3 hours under microwave irradiation. The reaction mixture was diluted with water (100 mL) and extracted with ethyl acetate (100 mL×3). The combined organic layers were washed with aq. saturated NaCl (100 mL×2), dried over anhyd. Na₂SO₄, filtered and concentrated. The residue was purified by flash silica gel chromatography (ISCOR; 12 g SepaFlash® Silica Flash Column, eluent of 0~47% ethyl acetate/petroleum ether gradient at 50 mL/min) to give 3-cyano-4-(3-methoxy-2,6-dimethylphenyl)-1,2-dimethyl-pyrrolo[2,3-b]pyridine-6-carboxamide (350 mg, 65% yield, 97% purity). LC-MS m/z 349 (M+H⁺).

### Step 8: Synthesis of 3-cyano-4-(3-hydroxy-2,6-dimethylphenyl)-1,2-dimethyl-pyrrolo[2,3-b]pyridine-6-carboxamide (compound 26)

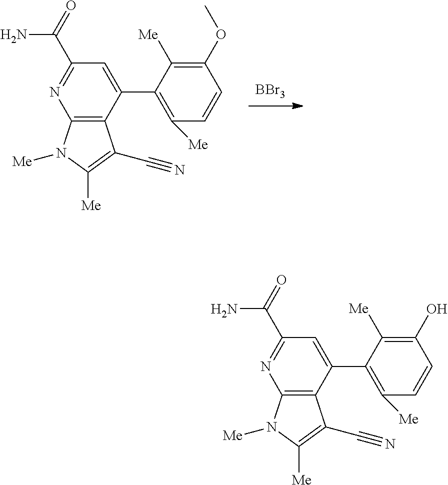

To a solution of 3-cyano-4-(3-methoxy-2,6-dimethylphenyl)-1,2-dimethyl-pyrrolo[2,3-b]pyridine-6-carboxamide (300 mg, 0.86 mmol, 1 eq) in DCM (2 mL) was added BBr₃ (0.1 mL). The mixture was stirred at 0° C. for 2 hours. The reaction mixture was quenched by the addition of water (10 mL) and adjusted to pH 8 with aq. saturated NaHCO₃ at 0° C. The mixture was extracted with DCM (25 mL×2). The combined organic layers were washed with aq. saturated NaCl (50 mL×2), dried over anhyd. Na₂SO₄, filtered and concentrated to give a residue. The residue was purified by RP-HPLC (column: Welch Xtimate C18 150×30 mm×5 um; mobile phase: [water(NH₃H₂O+NH₄HCO₃)-ACN]; B %: 10%-50%, 25 min) to give 3-cyano-4-(3-hydroxy-2,6-dimethylphenyl)-1,2-dimethyl-pyrrolo[2,3-b]pyridine-6-carboxamide (108.1 mg, 37% yield, 99.7% purity). LC-MS m/z 335 (M+H⁺). ¹H NMR (400 MHz, DMSO-d₆) δ=9.24 (s, 1H), 8.36 (d, 1H), 7.68 (d, 1H), 7.59 (s, 1H), 6.95 (d, 1H), 6.80 (d, 1H), 3.94 (s, 3H), 2.60 (s, 3H), 1.76 (s, 3H), 1.69 (s, 3H).

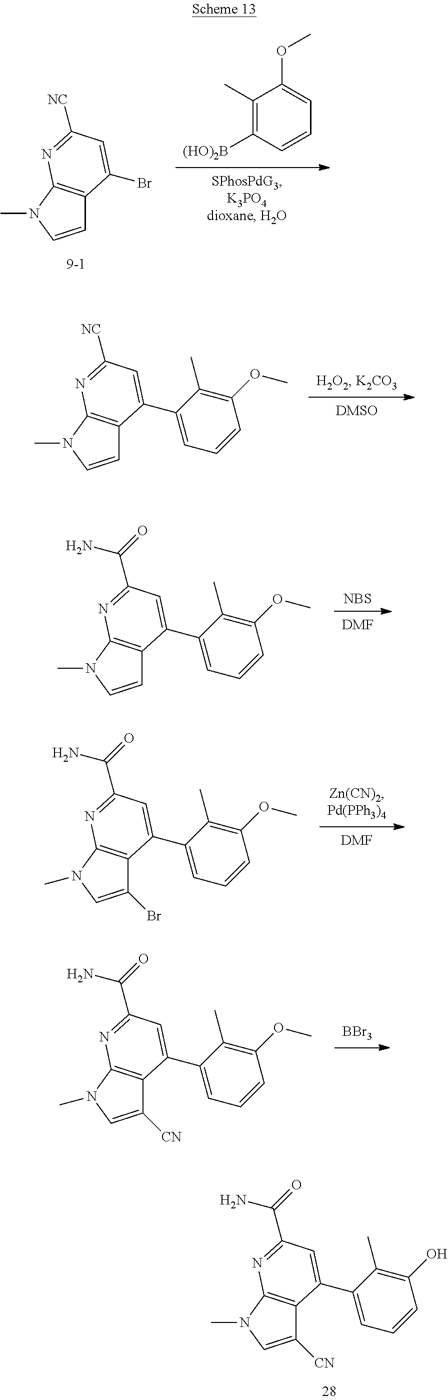

### Example 24. 3-Cyano-4-(3-hydroxy-2-methylphenyl)-1-methyl-1H-pyrrolo[2,3-b]pyridine-6-carboxamide (28)

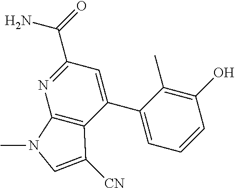 [SMILES: Cc1c(O)cccc1-c1cc(C(N)=O)nc2c1c(C#N)cn2C]

### Step 1: Synthesis of 4-(3-methoxy-2-methylphenyl)-1-methyl-pyrrolo[2,3-b]pyridine-6-carbonitrile

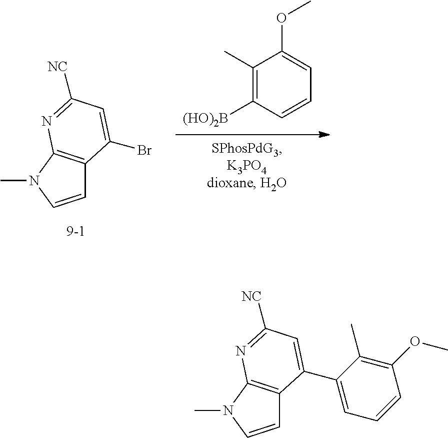

To a solution of 4-bromo-1-methyl-pyrrolo[2,3-b]pyridine-6-carbonitrile (0.5 g, 2.1 mmol, 1 eq), (3-methoxy-2-methylphenyl)boronic acid (422 mg, 2.5 mmol, 1.2 eq) in dioxane (5 mL) and H₂O (1 mL) was added K₃PO₄ (1.35 g, 6.35 mmol, 3 eq) and SPhos Pd G₃ (165 mg, 0.2 mmol, 0.1 eq). The reaction mixture was degassed with N₂ and stirred at 90° C. for 16 hours. The mixture was diluted with water (20 mL), extracted with EtOAc (20 mL×3). The combined organic layers were washed with aq. saturated NaCl (20 mL×2), dried over anhyd. Na₂SO₄, filtered and concentrated. The residue was purified by column chromatography (SiO₂, 0-50% Ethyl acetate in Petroleum ether) to give 4-(3-methoxy-2-methylphenyl)-1-methyl-pyrrolo[2,3-b]pyridine-6-carbonitrile (0.3 g). LC-MS m/z 278 (M+H⁺). ¹H NMR: (400 MHz, DMSO-d₆) δ=7.87 (d, 1H), 7.57 (s, 1H), 7.33-7.24 (m, 1H), 7.10 (d, 1H), 6.91 (d, 1H), 6.28 (d, 1H), 4.03 (s, 3H), 3.96 (m, 3H), 1.96 (s, 3H).

### Step 2: Synthesis of 4-(3-methoxy-2-methylphenyl)-1-methyl-pyrrolo[2,3-b]pyridine-6-carboxamide

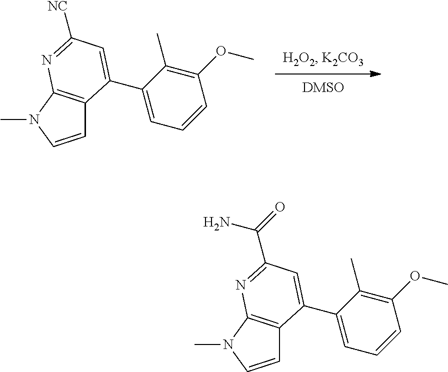

To a solution of 4-(3-methoxy-2-methylphenyl)-1-methyl-pyrrolo[2,3-b]pyridine-6-carbonitrile (0.4 g, 1.4 mmol, 1 eq) in DMSO (2 mL) was added K₂CO₃ (398 mg, 2.9 mmol, 2 eq) and H₂O₂ (0.6 mL, 30% purity) at 0° C. The mixture was stirred at 25° C. for 1 hour. The reaction mixture was diluted with water (40 mL), then adjusted to pH 7 by aq. HCl (2 M). The mixture was extracted with DCM (40 mL×3). The combined organic phase was washed with aq. saturated NaCl (30 mL×3) and anhyd. Na₂SO₃ (30 mL×3), dried over anhyd. Na₂SO₄, filtered and concentrated. The residue was purified by column chromatography (SiO₂, 0-10% MeOH in DCM) to give 4-(3-methoxy-2-methylphenyl)-1-methyl-pyrrolo[2,3-b]pyridine-6-carboxamide (0.2 g). LC-MS m/z 296 (M+H⁺).

### Step 3: Synthesis of 3-bromo-4-(3-methoxy-2-methylphenyl)-1-methyl-pyrrolo[2,3-b]pyridine-6-carboxamide

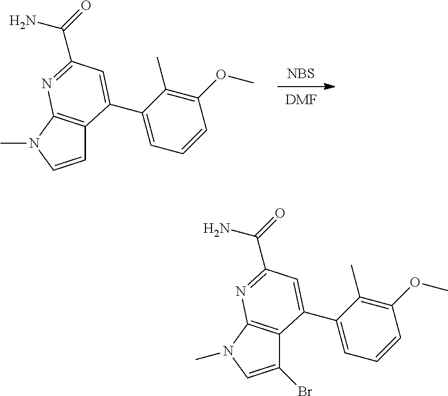

To a solution of 4-(3-methoxy-2-methylphenyl)-1-methyl-pyrrolo[2,3-b]pyridine-6-carboxamide (270 mg, 0.9 mmol, 1 eq) in DMF (2 mL) was added NBS (162 mg, 0.9 mmol, 1 eq). The mixture was stirred at 25° C. for 2 hours. The reaction mixture was diluted with water (40 mL), extracted with DCM (40 mL×3). The combined organic layers were washed with aq. saturated NaCl (30 mL×3) and anhyd. Na₂SO₃ (30 mL×3). The organic phase was dried over anhyd. Na₂SO₄, filtered and concentrated to give the residue. The residue was purified by column chromatography (SiO₂, 0-50% ethyl acetate in petroleum ether) to give 3-bromo-4-(3-methoxy-2-methylphenyl)-1-methyl-pyrrolo[2,3-b]pyridine-6-carboxamide (0.2 g). LC-MS m/z 376 (M+H⁺). ¹H NMR: (400 MHz, DMSO-d₆) δ=8.25 (br s, 1H), 7.96-7.92 (m, 1H), 7.64 (br s, 1H), 7.55 (s, 1H), 7.30-7.22 (m, 1H), 7.05 (d, 1H), 6.79 (d, 1H), 3.96-3.93 (m, 3H), 3.85 (s, 3H), 1.81 (s, 3H).

### Step 4: Synthesis of 3-cyano-4-(3-methoxy-2-methylphenyl)-1-methyl-pyrrolo[2,3-b]pyridine-6-carboxamide

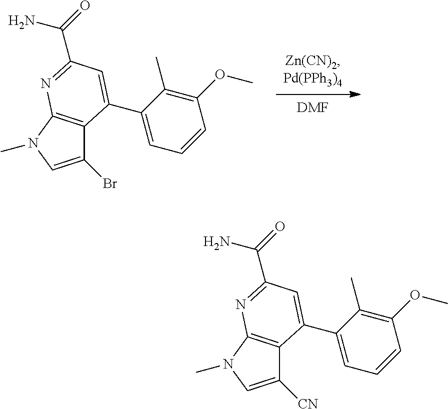

To a solution of 3-bromo-4-(3-methoxy-2-methylphenyl)-1-methyl-pyrrolo[2,3-b]pyridine-6-carboxamide (0.45 g, 1.2 mmol, 1 eq) in DMF (5 mL) was added Pd(PPh₃)₄ (278 mg, 0.25 mmol, 0.2 eq) and Zn(CN)₂ (1.4 g, 12.02 mmol, 10 eq). The mixture was degassed with N₂ and stirred at 160° C. for 3 hours under microwave irradiation. The reaction mixture was diluted with water (30 mL), extracted with ethyl acetate (50 mL×3). The combined organic layers were washed with aq. saturated NaCl (50 mL×2), dried over anhyd. Na₂SO₄, filtered and concentrated. The residue was purified by column chromatography (SiO₂, 0-5% MeOH in DCM) to afford compound 3-cyano-4-(3-methoxy-2-methylphenyl)-1-methyl-pyrrolo[2,3-b]pyridine-6-carboxamide (0.2 g). LC-MS m/z 321 (M+H⁺).

### Step 5: Synthesis of 3-cyano-4-(3-hydroxy-2-methylphenyl)-1-methyl-pyrrolo[2,3-b]pyridine-6-carboxamide (compound 28)

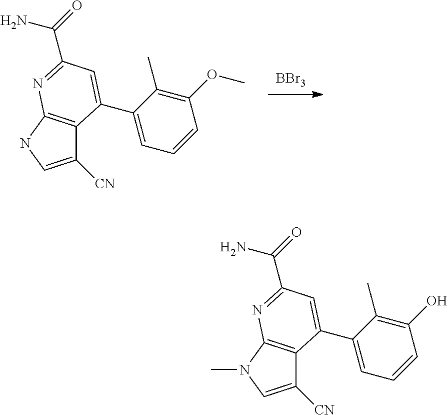

To a solution of 3-cyano-4-(3-methoxy-2-methylphenyl)-1-methyl-pyrrolo[2,3-b]pyridine-6-carboxamide (0.2 g, crude) in DCM (3 mL) was added BBr₃ (1 M, 3 mL, in DCM) at 0° C. and the mixture was stirred at 25° C. for 2 hours. The reaction mixture was quenched with water (50 mL) at 20° C., adjusted pH to 7 by aq. saturated NaHCO₃ at 0° C. The mixture was extracted with DCM (30 mL×3) and the combined organic layers were washed with aq. saturated NaCl (20 mL×2), dried over anhyd. Na₂SO₄, filtered and concentrated. The crude product was purified by RP-HPLC (column: Xtimate C18 150×40 mm×10 um; mobile phase: [water(FA)-ACN]:B %: 4%-44%, 36 min) to give 3-cyano-4-(3-hydroxy-2-methylphenyl)-1-methyl-pyrrolo[2,3-b]pyridine-6-carboxamide (6.3 mg, 96.6% purity). LC-MS m/z 307 (M+H⁺). ¹H NMR: (400 MHz, DMSO-d₆) δ=9.53 (s, 1H), 8.62 (s, 1H), 8.32 (s, 1H) 7.71 (s, 2H), 7.10 (t, 1H), 6.92 (d, 1H), 6.69 (d, 1H), 4.01 (s, 3H), 1.86 (s, 3H).

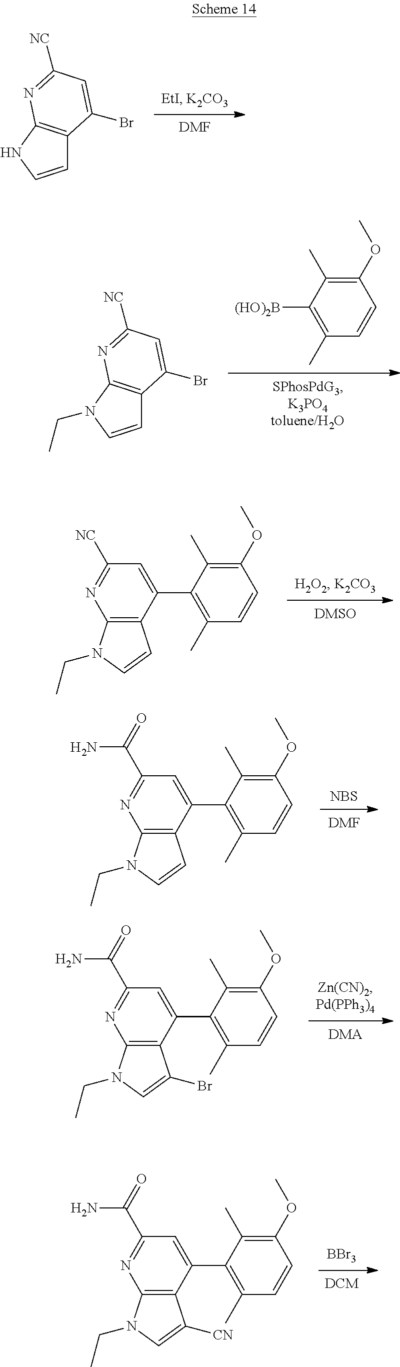

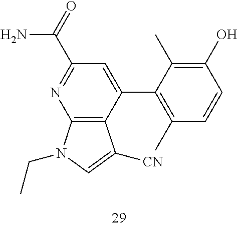 [SMILES: CCn1cc(C#N)c2c(-c3c(C)ccc(O)c3C)cc(C(N)=O)nc21]

### Example 25. 3-Cyano-1-ethyl-4-(3-hydroxy-2,6-dimethylphenyl)-1H-pyrrolo[2,3-b]pyridine-6-carboxamide (29)

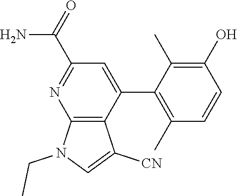 [SMILES: CCn1cc(C#N)c2c(-c3c(C)ccc(O)c3C)cc(C(N)=O)nc21]

### Step 1: Synthesis of 4-bromo-1-ethyl-pyrrolo[2,3-b]pyridine-6-carbonitrile

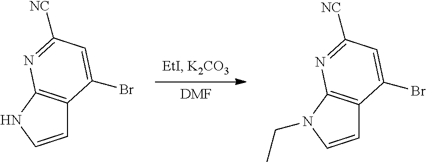

To a solution of 4-bromo-1H-pyrrolo[2,3-b]pyridine-6-carbonitrile (2 g, 9.01 mmol, 1 eq) in DMF (20 mL) was added K₂CO₃ (3.73 g, 27.02 mmol, 3 eq) and EtI (1.68 g, 10.77 mmol, 0.86 mL, 1.2 eq) at 0° C. The mixture was stirred at 25° C. for 17 hours. The reaction mixture was quenched with aq. saturated NH₄Cl (30 mL), diluted with water (150 mL), followed by extraction with EtOAc (100 mL×3). The organic layer was dried over anhyd. Na₂SO₄, filtered and concentrated to give a crude product 4-bromo-1-ethyl-pyrrolo[2,3-b]pyridine-6-carbonitrile (2.02 g, crude). LC-MS m/z 252 (M+H⁺).

### Step 2: Synthesis of 1-ethyl-4-(3-methoxy-2,6-dimethylphenyl)pyrrolo[2,3-b]pyridine-6-carbonitrile

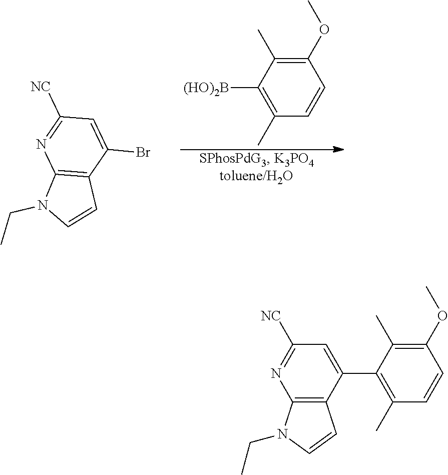

To a solution of 4-bromo-1-ethyl-pyrrolo[2,3-b]pyridine-6-carbonitrile (1.95 g, 7.80 mmol, 1 eq) and (3-methoxy-2,6-dimethylphenyl)boronic acid (2.81 g, 15.60 mmol, 2 eq) in toluene (30 mL) and H₂O (6 mL) was added SPhos Pd G3 (608 mg, 0.78 mmol, 0.1 eq) and K₃PO₄ (6.62 g, 31.19 mmol, 4 eq). The mixture was degassed with N₂ and stirred at 80° C. for 16 hours. The reaction mixture was concentrated, then diluted with water (50 mL), followed by extraction with EtOAc (50 mL×3). The organic phase was washed by aq. saturated NaCl 100 mL (50 mL×2), dried over anhyd. Na₂SO₄, filtered and concentrated. The crude product was purified by flash silica gel chromatography (ISCO®; 40 g SepaFlash® Silica Flash Column, Eluent of 0~64% ethyl acetate/petroleum ether gradient at 30 m/min) to give 1-ethyl-4-(3-methoxy-2,6-dimethylphenyl)pyrrolo[2,3-b]pyridine-6-carbonitrile (2.52 g, 88.4% purity). LC-MS m/z 306 (M+H⁺).

### Step 3: Synthesis of 1-ethyl-4-(3-methoxy-2,6-dimethylphenyl)pyrrolo[2,3-b]pyridine-6-carboxamide

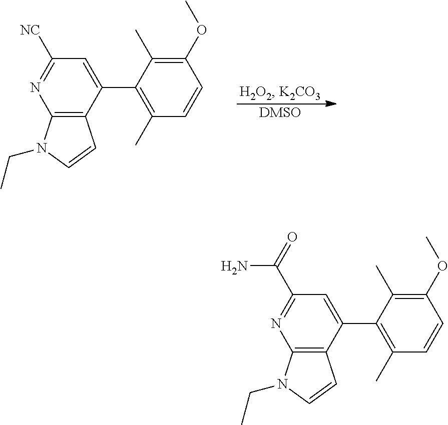

To a solution of 1-ethyl-4-(3-methoxy-2,6-dimethylphenyl)pyrrolo[2,3-b]pyridine-6-carbonitrile (2.3 g, 7.53 mmol, 1 eq) in DMSO (40 mL) was added K₂CO₃ (1.56 g, 11.32 mmol, 1.5 eq) and H₂O₂ (12 mL, 30% purity) at 0° C. The mixture was stirred at 25° C. for 1 hour. The reaction mixture was diluted with water (200 mL) and filtered. The filter cake was triturated with water (20 mL) to give 1-ethyl-4-(3-methoxy-2,6-dimethylphenyl)pyrrolo[2,3-b]pyridine-6-carboxamide (2.13 g, 76% yield, 87.4% purity). LC-MS m/z 325 (M+H⁺).

### Step 4: Synthesis of 3-bromo-1-ethyl-4-(3-methoxy-2,6-dimethylphenyl)pyrrolo[2,3-b]pyridine-6-carboxamide

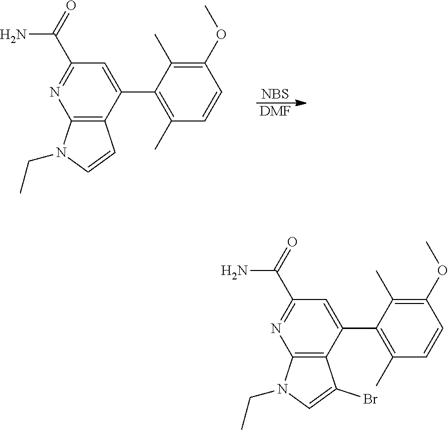

To a solution of 1-ethyl-4-(3-methoxy-2,6-dimethylphenyl)pyrrolo[2,3-b]pyridine-6-carboxamide (1 g, 3.09 mmol, 1 eq) in DMF (10 mL) was added NBS (550 mg, 3.09 mmol, 0.1 eq). The mixture was stirred at 25° C. for 1 hour. The mixture was diluted with H₂O (150 mL), then was filtered to get the filter cake. The filter cake was triturated with methanol (5 mL) to give 3-bromo-1-ethyl-4-(3-methoxy-2,6-dimethylphenyl)pyrrolo[2,3-b]pyridine-6-carboxamide (423 mg, crude). LC-MS m/z 404 (M+H⁺). ¹H NMR: (400 MHz, DMSO-d₆) (3=8.26 (s, 1H), 7.99 (s, 1H), 7.57-7.71 (m, 1H), 7.48 (s, 1H), 7.10 (d, 1H), 6.94 (d, 1H), 4.45 (m, 2H), 3.80 (s, 3H), 1.77 (s, 3H), 1.70 (s, 3H), 1.45 (t, 3H).

### Step 5: Synthesis of 3-cyano-1-ethyl-4-(3-methoxy-2,6-dimethylphenyl)pyrrolo[2,3-b]pyridine-6-carboxamide

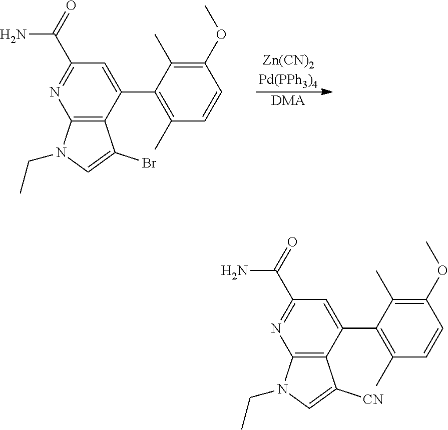

To a solution of 3-bromo-1-ethyl-4-(3-methoxy-2,6-dimethylphenyl)pyrrolo[2,3-b]pyridine-6-carboxamide (373 mg, 0.93 mmol, 1 eq) in DMA (8 mL) was added Pd(PPh₃)₄ (216 mg, 0.19 mmol, 0.2 eq) and Zn(CN)₂ (1.33 g, 11.33 mmol, 12.2 eq). The mixture was degassed with N₂ and stirred at 160° C. for 3 hours under microwave irradiation. The reaction mixture was diluted with water (50 mL) and extracted with EtOAc (30 mL×3). The organic phase was washed by aq. saturated NaCl (20 mL×2), dried over anhyd. Na₂SO₄, filtered and concentrated under vacuum. The crude product was purified by flash silica gel chromatography (ISCO®; 4 g SepaFlash® Silica Flash Column, Eluent of 0~3% methanol/dichloromethane at 30 mL/min) to afford 3-cyano-1-ethyl-4-(3-methoxy-2,6-dimethylphenyl)pyrrolo[2,3-b]pyridine-6-carboxamide (164 mg). LC-MS m/z 349 (M+H⁺).

### Step 6: Synthesis of 3-cyano-1-ethyl-4-(3-hydroxy-2,6-dimethylphenyl)pyrrolo[2,3-b]pyridine-6-carboxamide (compound 29)

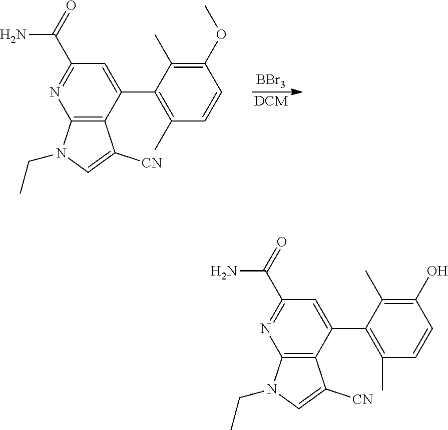

To a solution of 3-cyano-1-ethyl-4-(3-methoxy-2,6-dimethylphenyl)pyrrolo[2,3-b]pyridine-6-carboxamide (100 mg, crude) in DCM (2 mL) was added BBr₃ (0.1 mL). The mixture was stirred at 0° C. for 1 hour. The reaction solution was quenched by the addition of water (20 mL), adjusted to pH=8 with aq. NH₃·H₂O, and extracted with DCM (10 mL×3). The combined organic layers were washed with aq. saturated NaCl 20 mL (10 mL×2), dried over anhyd. Na₂SO₄, filtered and concentrated. The crude product was purified by RP-HPLC (column: Xtimate C18 150×40 mm×10 um; mobile phase: [water(NH₃H₂O+NH₄HCO₃)-ACN]; B %: 12%-52%, 25 min) to give 3-cyano-1-ethyl-4-(3-hydroxy-2,6-dimethylphenyl)pyrrolo[2,3-b]pyridine-6-carboxamide (10.9 mg, 32.05 μmol, 98.32% purity). LC-MS m/z 335 (M+H⁺). ¹H NMR: (400 MHz, DMSO-d₆) δ=9.25 (s, 1H), 8.69 (s, 1H), 8.33 (s, 1H), 7.68-7.87 (m, 1H), 7.64 (s, 1H), 6.89-7.06 (m, 1H), 6.82 (s, 1H), 4.50 (m, 2H), 1.77 (s, 3H), 1.70 (s, 3H), 1.49 (t, 3H).

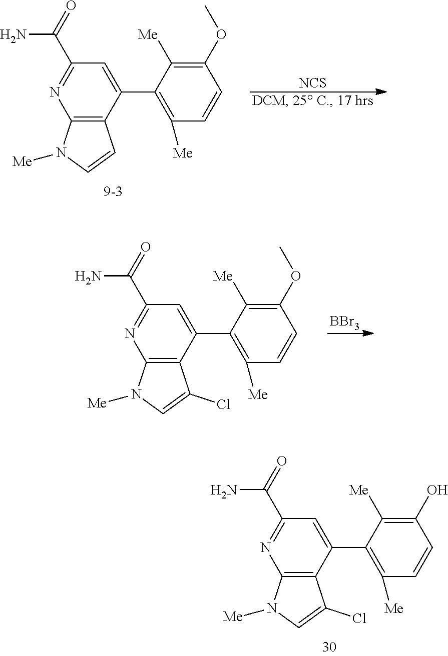

### Example 26. 3-Chloro-4-(3-hydroxy-2,6-dimethylphenyl)-1-methyl-1H-pyrrolo[2,3-b]pyridine-6-carboxamide (30)

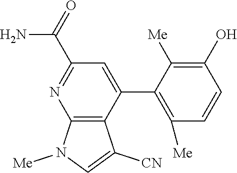 [SMILES: Cc1ccc(O)c(C)c1-c1cc(C(N)=O)nc2c1c(C#N)cn2C]

### Step 1. Synthesis of 3-chloro-4-(3-methoxy-2,6-dimethylphenyl)-1-methyl-pyrrolo[2,3-b]pyridine-6-carboxamide

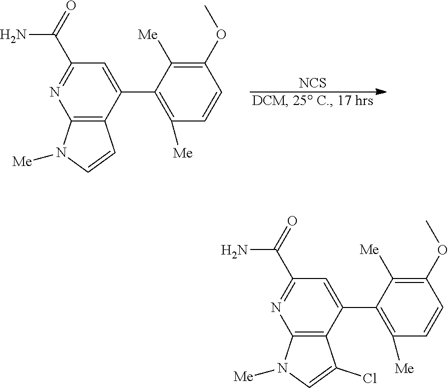

To a solution of 4-(3-methoxy-2,6-dimethylphenyl)-1-methyl-pyrrolo[2,3-b]pyridine-6-carboxamide (9-3, 300 mg, 0.96 mmol, 1 eq) in DCM (4 mL) was added 1-chloropyrrolidine-2,5-dione (129 mg, 0.96 mmol, 1 eq). The mixture was stirred at 25° C. for 17 hours. The reaction mixture was concentrated to give a residue, which was purified by column chromatography (SiO₂, PE/EA=1/1) to give 3-chloro-4-(3-methoxy-2,6-dimethylphenyl)-1-methyl-pyrrolo[2,3-b]pyridine-6-carboxamide (0.3 g, 84% yield, 94% purity). LC-MS m/z 344 (M+H⁺). ¹H NMR: (400 MHz, DMSO-d₆) δ=9.21 (s, 1H), 7.95 (s, 1H), 7.51 (s, 1H), 6.93 (d, 1H), 6.79 (d, 1H), 3.90 (d, 6H), 1.75 (s, 3H), 1.68 (s, 3H).

### Step 2. Synthesis of 3-chloro-4-(3-hydroxy-2,6-dimethylphenyl)-1-methyl-1H-pyrrolo[2,3-b]pyridine-6-carboxamide (compound 30)

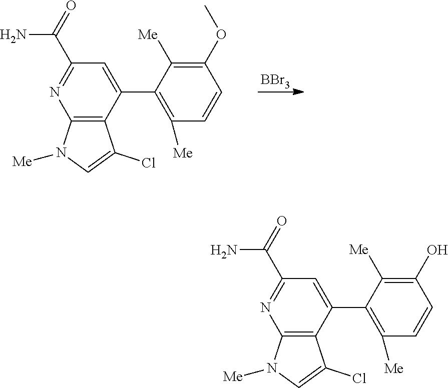

To a solution of 3-chloro-4-(3-methoxy-2,6-dimethylphenyl)-1-methyl-pyrrolo[2,3-b]pyridine-6-carboxamide (100 mg, 0.29 mmol, 1 eq) in DCM (2 mL) was added BBr₃ (0.1 mL) at 0° C. The mixture was stirred at 20° C. for 0.5 hour. The reaction mixture was quenched by H₂O (10 mL) extracted with EtOAc (30 mL×3). The combined organic layers were washed with aq. saturated NaCl (100 mL×2), dried over anhyd. Na₂SO₄, filtered and concentrated. The residue was purified by RP-HPLC (column: Xtimate C18 150×40 mm×10 um; mobile phase: [water(HCl)-ACN]; B %: 16%-56%, 36 min) to give 3-chloro-4-(3-hydroxy-2,6-dimethylphenyl)-1-methyl-pyrrolo[2,3-b]pyridine-6-carboxamide (7.3 mg, 0.02 mmol). LC-MS m/z 330 (M+H⁺).

¹H NMR: (DMSO-d₆) δ=9.20 (s, 1H), 8.26 (d, 1H), 7.86 (s, 1H), 7.65 (d, 1H), 7.46 (s, 1H), 6.93 (d, 1H), 6.78 (d, 1H), 3.94 (s, 3H), 1.81-1.61 (m, 6H).

### Example 27. (M)-3-Chloro-4-(3-hydroxy-2,6-dimethylphenyl)-1-methyl-1H-pyrrolo[2,3-b]pyridine-6-carboxamide (31) and (P)-3-chloro-4-(3-hydroxy-2,6-dimethylphenyl)-1-methyl-1H-pyrrolo[2,3-b]pyridine-6-carboxamide (32)

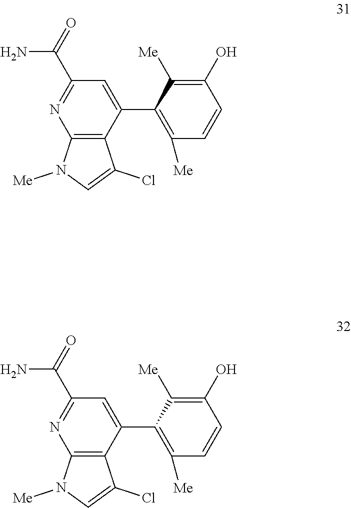

Compound 30 (60 mg, of racemate) was resolved by SFC separation (column: DAICEL CHIRALPAK IG (250 mm×30 mm, 10 um); mobile phase: [0.1% NH₃H₂O MeOH]; B %: 55%-55%, min) to give compound 31 and compound 32: (M)-3-chloro-4-(3-hydroxy-2,6-dimethylphenyl)-1-methyl-1H-pyrrolo[2,3-b]pyridine-6-carboxamide and (P)-3-chloro-4-(3-hydroxy-2,6-dimethylphenyl)-1-methyl-1H-pyrrolo[2,3-b]pyridine-6-carboxamide.

Peak 1: SFC analysis condition: t_{R}=0.378 min in Chiralpak IG-3 50×4.6 mm I.D., 3 um Mobile phase: 40% of Methanol (0.05% DEA) in CO₂ Flow rate: 4 ml/min Column temp.:35° C. ABPR: 1500 psi. LC-MS m/z 330 (M+H⁺). ¹H NMR: (400 MHz, DMSO-d₆) δ=9.20 (s, 1H), 8.26 (d, 1H), 7.86 (s, 1H), 7.65 (d, 1H), 7.46 (s, 1H), 6.93 (d, 1H), 6.78 (d, 1H), 3.94 (s, 3H), 1.78-1.66 (m, 6H).

Peak 2: SFC analysis condition: t_{R}=2.170 min in Chiralpak IG-3 50×4.6 mm I.D., 3 um Mobile phase: 40% of Methanol (0.05% DEA) in CO₂ Flow rate: 4 ml/min Column temp.:35° C. ABPR: 1500 psi. LC-MS m/z 330 (M+H⁺). ¹H NMR: (400 MHz, DMSO-d₆) δ=9.20 (s, 1H), 8.27 (d, 1H), 7.86 (s, 1H), 7.65 (d, 1H), 7.47 (s, 1H), 6.93 (d, 1H), 6.78 (d, 1H), 3.94 (s, 3H), 1.79-1.62 (m, 6H).

### Example 28. (M)-3-Cyano-4-(3-hydroxy-2,6-dimethylphenyl)-1,2-dimethyl-1H-pyrrolo[2,3-b]pyridine-6-carboxamide (33) and (P)-3-cyano-4-(3-hydroxy-2,6-dimethylphenyl)-1,2-dimethyl-1H-pyrrolo[2,3-b]pyridine-6-carboxamide (34)

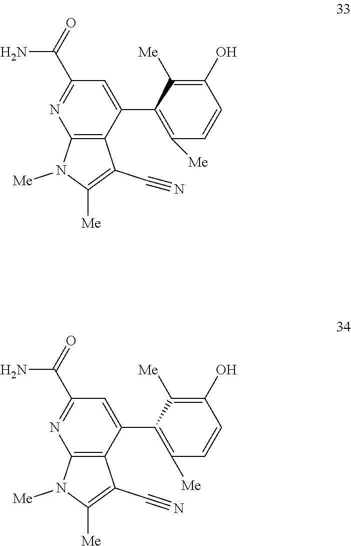

Compound 26 (96 mg, racemate) was resolved by SFC separation (column: DAICEL CHIRALPAK AD(250 mm×30 mm, 10 um); mobile phase: [0.1% NH₃H₂O EtOH]; B %: 55%-55%, min) to give compound 33 and compound 34: (M)-3-cyano-4-(3-hydroxy-2,6-dimethylphenyl)-1,2-dimethyl-1H-pyrrolo[2,3-b]pyridine-6-carboxamide and (P)-3-cyano-4-(3-hydroxy-2,6-dimethylphenyl)-1,2-dimethyl-1H-pyrrolo[2,3-b]pyridine-6-carboxamide.

Peak 1. 33.3 mg. SFC t_{R}=1.473 min in Column: Chiralcel OJ-3 50×4.6 mm I.D., 3 um Mobile phase: A: CO₂ B: Ethanol (0.05% DEA) Gradient: from 5% to 40% of B in 2.5 min and hold 40% of 0.5 min, then 5% of B for 1 min Flow rate: 4 ml/min Column temp.:35° C. ABPR: 1500 psi. LC-MS m/z 335 (M+H⁺). ¹H NMR (400 MHz, DMSO-d₆) δ=9.24 (s, 1H), 8.36 (d, 1H), 7.68 (d, 1H), 7.59 (s, 1H), 6.95 (d, 1H), 6.80 (d, 1H), 3.94 (s, 3H), 2.60 (s, 3H), 1.76 (s, 3H), 1.69 (s, 3H).

Peak 2. 28.9 mg. SFC t_{R}=1.724 min in Column: Chiralcel OJ-3 50×4.6 mm I.D., 3 um Mobile phase: A: CO₂ B: Ethanol (0.05% DEA) Gradient: from 5% to 40% of B in 2.5 min and hold 40% of 0.5 min, then 5% of B for 1 min Flow rate: 4 mL/min Column temp: 35° C. ABPR: 1500 psi. LC-MS m/z 335 (M+H⁺). ¹H NMR (400 MHz, DMSO-d₆) δ=9.23 (s, 1H), 8.36 (d, 1H), 7.68 (s, 1H), 7.59 (s, 1H), 6.95 (d, 1H), 6.80 (d, 1H), 3.94 (s, 3H), 2.60 (s, 3H), 1.76 (s, 3H), 1.69 (s, 3H).

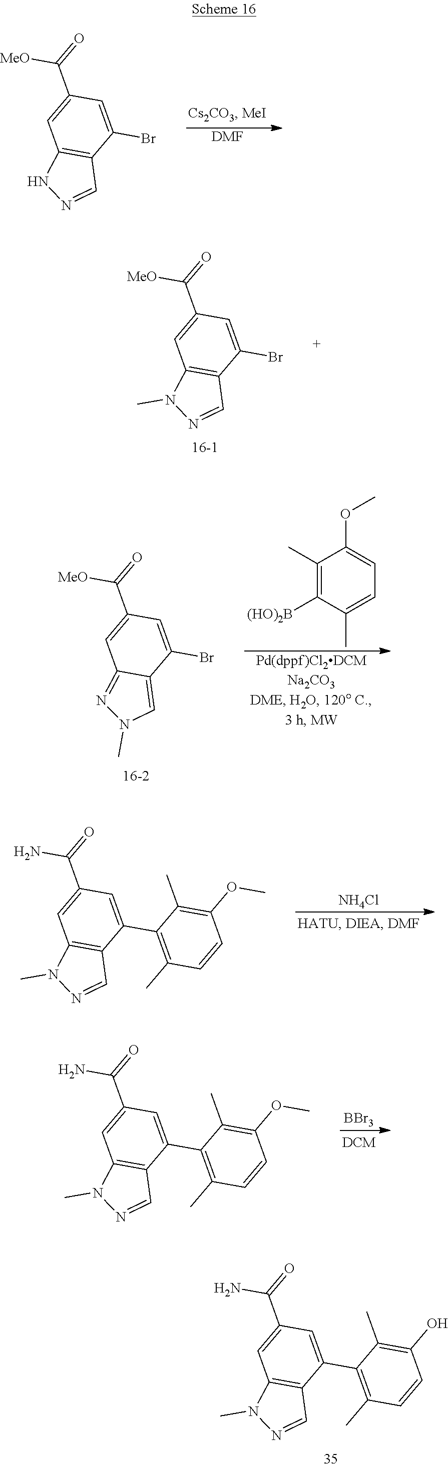

### Example 29. 4-(3-Hydroxy-2,6-dimethylphenyl)-1-methyl-1H-indazole-6-carboxamide (35)

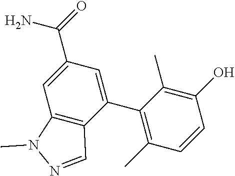 [SMILES: Cc1ccc(O)c(C)c1-c1cc(C(N)=O)cc2c1cnn2C]

### Step 1: Synthesis of methyl 4-bromo-1-methyl-indazole-6-carboxylate and methyl 4-bromo-2-methyl-indazole-6-carboxylate

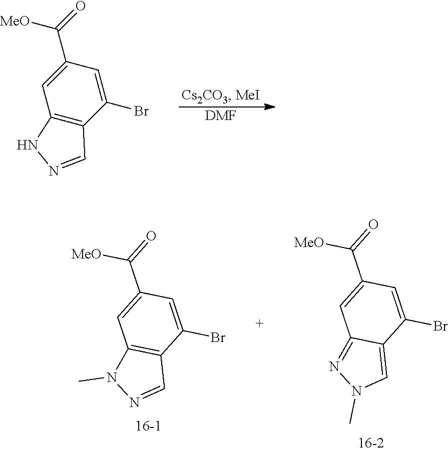

To a solution of methyl 4-bromo-1H-indazole-6-carboxylate (1 g, 3.92 mmol, 1 eq) in DMF (15 mL) was added Cs₂CO₃ (2.55 g, 7.84 mmol, 2 eq) and MeI (0.3 mL, 1.3 eq). The mixture was stirred at 15° C. for 24 hours. The mixture was diluted with water (100 mL) and extracted with EtOAc (60 mL×3). The combined organic layers were washed with aq. saturated. NaCl (60 mL×2), dried over anhyd. Na₂SO₄, filtered and concentrated to give a residue. The residue was purified by column chromatography (SiO₂, 0-20% Ethyl acetate in Petroleum ether) to give methyl 4-bromo-1-methyl-indazole-6-carboxylate (16-1, 435 mg, 41% yield, 99% purity), methyl 4-bromo-2-methyl-indazole-6-carboxylate (16-2, 258 mg, 24% yield, 99% purity). 16-1: LC-MS m/z 269 (M+H⁺). ¹H NMR: (400 MHz, DMSO-d₆) δ=8.32 (s, 1H), 8.12 (s, 1H), 7.79 (s, 1H), 4.15 (s, 3H), 3.91 (s, 3H), 16-2: LC-MS m/z 269 (M+H⁺). ¹H NMR: (400 MHz, DMSO-d₆) δ=8.55 (s, 1H), 8.27 (s, 1H), 7.71 (s, 1H), 4.24 (s, 3H), 3.88 (s, 3H).

### Step 2: Synthesis of 4-(3-methoxy-2,6-dimethylphenyl)-1-methyl-indazole-6-carboxylic acid

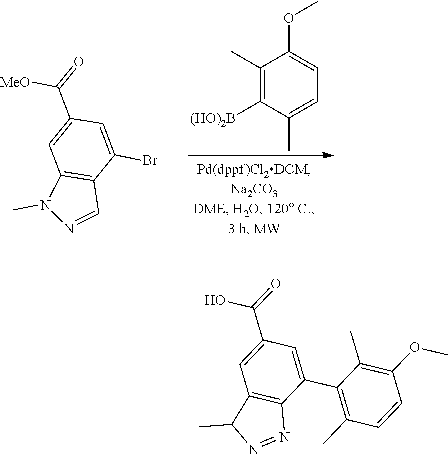

To a solution of methyl 4-bromo-1-methyl-indazole-6-carboxylate (200 mg, 0.74 mmol, 1 eq) and (3-methoxy-2,6-dimethylphenyl) boronic acid (200 mg, 1.11 mmol, 1.5 eq) in DME (3 mL) and H₂O (1 mL) was added Pd(dppf)Cl₂·DCM (60 mg, 0.07 mmol, 0.1 eq) and Na₂CO₃ (787 mg, 7.43 mmol, 10 eq). The mixture was degassed with N₂ and stirred at 120° C. for 3 hours under microwave irradiation. The reaction mixture was adjusted to pH 4 with aq. HCl (1 M). The mixture was filtered to give the filter cake, which was concentrated to give 4-(3-methoxy-2,6-dimethylphenyl)-1-methyl-indazole-6-carboxylic acid (100 mg, crude). LC-MS m/z 311 (M+H⁺).

### Step 3: Synthesis of 4-(3-methoxy-2,6-dimethylphenyl)-1-methyl-indazole-6-carboxamide

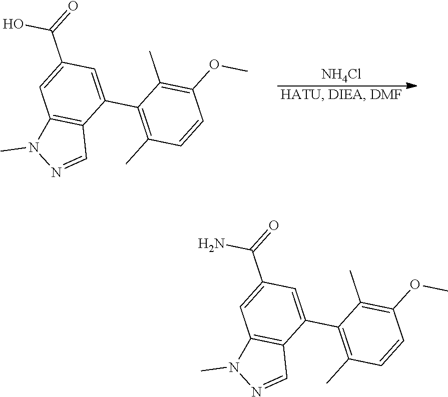

To a solution of 4-(3-methoxy-2,6-dimethylphenyl)-1-methyl-indazole-6-carboxylic acid (80 mg, 0.25 mmol, 1 eq) and NH₄Cl (20 mg, 0.38 mmol, 1.5 eq) in DMF (1 mL) was added HATU (117 mg, 0.31 mmol, 1.2 eq) and DIEA (100 mg, 0.77 mmol, 3 eq). The mixture was stirred at 15° C. for 16 hours. The reaction mixture was diluted with water (50 mL) and extracted with EtOAc (50 mL×3). The combined organic layers were washed with aq. saturated. NaCl (60 mL×2), dried over anhyd. Na₂SO₄, filtered and concentrated to give 4-(3-methoxy-2,6-dimethylphenyl)-1-methyl-indazole-6-carboxamide (100 mg, crude). LC-MS m/z 310 (M+H⁺).

### Step 4: Synthesis of 4-(3-hydroxy-2,6-dimethylphenyl)-1-methyl-indazole-6-carboxamide (compound 35)

To a solution of 4-(3-methoxy-2,6-dimethylphenyl)-1-methyl-indazole-6-carboxamide (90 mg, crude) in DCM (2 mL) was added BBr₃ (0.1 mL) at 0° C. under N₂. The mixture was stirred at 0° C. for 1 hour and 15° C. for another 1 hour. The reaction mixture was adjusted to pH 7 with aq. saturated NaHCO₃, diluted with water (50 mL) and extracted with DCM (50 mL×3). The combined organic layers were dried over anhyd. Na₂SO₄, filtered and concentrated to give a residue. The crude product was purified by RP-HPLC (column: Welch Xtimate C18 150×40 mm×10 um; mobile phase: [water(NH₃H₂O+NH₄HCO₃)-ACN]; B %: 4%-44%. 30 min) to give 4-(3-hydroxy-2,6-dimethylphenyl)-1-methyl-indazole-6-carboxamide (19.1 mg, 99% purity). LC-MS m/z 296 (M+H⁺). ¹H NMR: (400 MHz, DMSO-d₆) δ=9.25 (s, 1H), 8.19 (s, 1H), 8.09 (s, 1H), 7.52 (s, 1H), 7.46 (s, 1H), 7.38 (s, 1H), 6.96 (d, 1H), 6.79 (d, 1H), 4.12 (s, 3H), 1.78 (s, 3H), 1.70 (s, 3H).

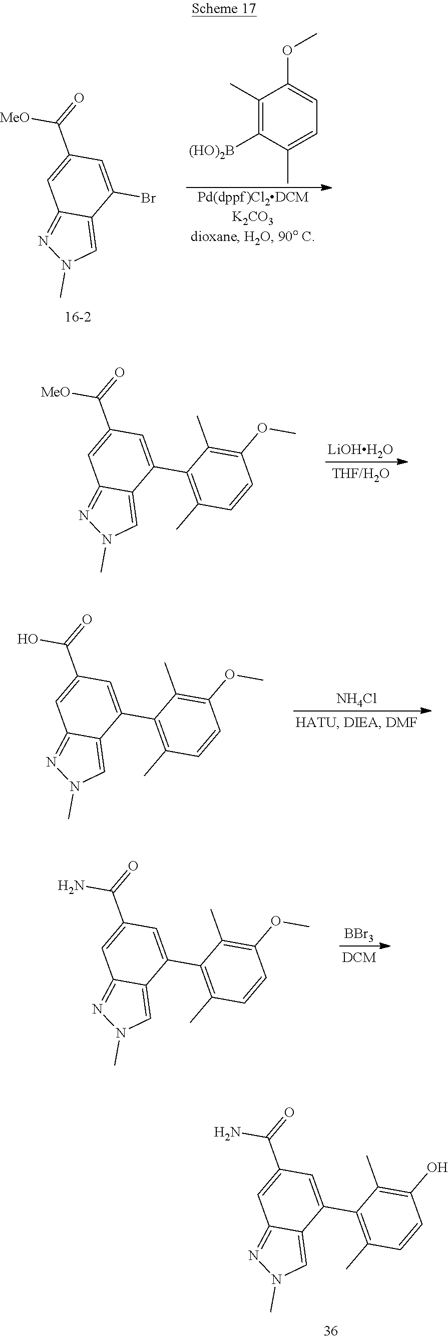

### Example 30. 4-(3-Hydroxy-2,6-dimethylphenyl)-2-methyl-2H-indazole-6-carboxamide (36)

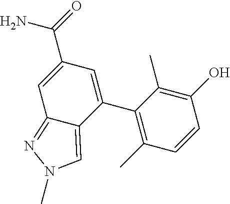 [SMILES: Cc1ccc(O)c(C)c1-c1cc(C(N)=O)cc2nn(C)cc12]

### Step 1: Synthesis of 4-(3-methoxy-2,6-dimethylphenyl)-2-methyl-indazole-6-carboxylate

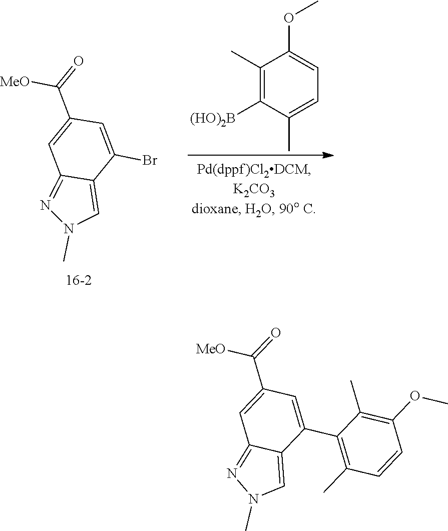

To a solution of methyl 4-bromo-2-methyl-indazole-6-carboxylate (200 mg, 0.7 mmol, 1 eq) and (3-methoxy-2,6-dimethylphenyl)boronic acid (161 mg, 0.9 mmol, 1.2 eq) in dioxane (4 mL) and H₂O (1 mL) was added Pd(dppf)Cl₂·CH₂Cl₂ (61 mg, 0.074 mmol, 0.1 eq) and K₂CO₃ (308 mg, 2.23 mmol, 3 eq). The mixture was stirred at 90° C. under N₂ for 2 hours. The reaction mixture was diluted with water (40 mL) at 25° C., extracted with ethyl acetate (30 mL×3). The combined organic layers were washed with aq. saturated NaCl (20 mL×3), dried over anhyd. Na₂SO₄, filtered and concentrated to give methyl 4-(3-methoxy-2,6-dimethylphenyl)-2-methyl-indazole-6-carboxylate (520 mg, crude). LC-MS m/z 325 (M+H⁺).

### Step 2: Synthesis of 4-(3-methoxy-2,6-dimethylphenyl)-2-methyl-indazole-6-carboxylic acid

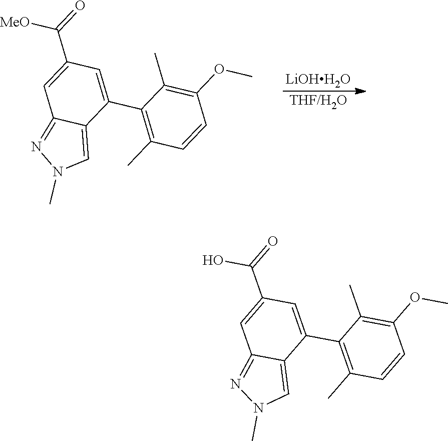 [SMILES: COC(=O)c1cc(-c2c(C)ccc(OC)c2C)c2cn(C)nc2c1 ; "LiOH·H2O" ; "THF/H2O" ; COc1ccc(C)c(-c2cc(C(=O)O)cc3nn(C)cc23)c1C]

To a solution of methyl 4-(3-methoxy-2,6-dimethylphenyl)-2-methyl-indazole-6-carboxylate (500 mg, crude) in THF (1 mL) and H₂O (1 mL) was added LiOH·H₂O (194 mg, 4.62 mmol). The mixture was stirred at 25° C. for 16 hours. The reaction mixture was poured into water (40 mL) and neutralized to pH 6-7 with aq. NaHCO₃. The mixture was extracted with Ethyl acetate (30 mL×3). The combined organic layers were washed with aq. saturated NaCl (20 mL×3), dried over anhyd. Na₂SO₄, filtered and concentrated. The residue was purified by column chromatography on silica gel (0%-30% Ethyl acetate in Petroleum ether) to give 4-(3-methoxy-2,6-dimethylphenyl)-2-methyl-indazole-6-carboxylic acid (70 mg, 95% purity). LC-MS m/z 311 (M+H⁺).

### Step 3: Synthesis of 4-(3-methoxy-2,6-dimethylphenyl)-2-methyl-indazole-6-carboxamide

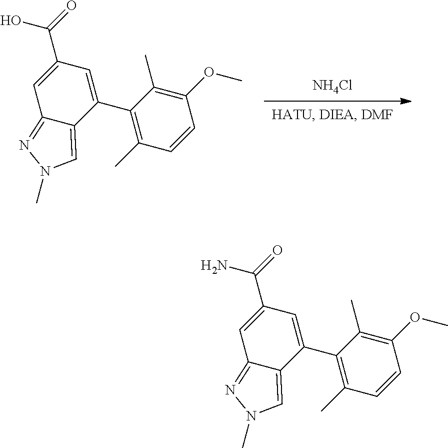 [SMILES: COc1ccc(C)c(-c2cc(C(=O)O)cc3nn(C)cc23)c1C ; "HATU, DIEA, DMF" ; "NH4Cl" ; COc1ccc(C)c(-c2cc(C(N)=O)cc3nn(C)cc23)c1C]

To a solution of 4-(3-methoxy-2,6-dimethylphenyl)-2-methyl-indazole-6-carboxylic acid (50 mg, 0.161 mmol, 1 eq) and NH₄Cl (13 mg, 0.241 mmol, 1.5 eq) in DMF (1 mL) was added DIEA (62 mg, 0.483 mmol, 3 eq) and HATU (92 mg, 0.242 mmol, 1.5 eq). The mixture was stirred at 25° C. for 16 hours. The reaction mixture was diluted with water (40 mL), extracted with Ethyl acetate (30 mL×3). The combined organic layers were washed with aq. saturated NaCl (20 mL×3), dried over anhyd. Na₂SO₄, filtered and concentrate to give 4-(3-methoxy-2,6-dimethylphenyl)-2-methyl-indazole-6-carboxamide (100 mg, crude). LC-MS m/z 310 (M+H⁺).

### Step 4: Synthesis of 4-(3-hydroxy-2,6-dimethylphenyl)-2-methyl-indazole-6-carboxamide (compound 36)

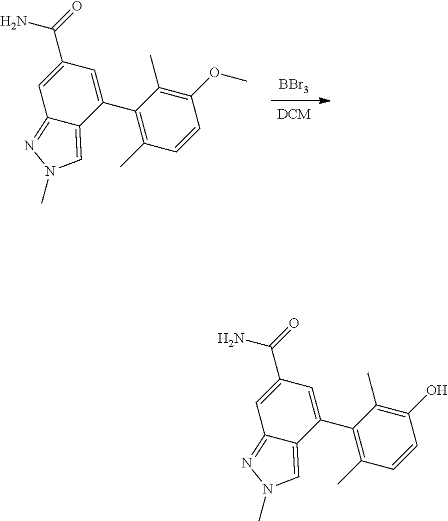

To a solution of 4-(3-methoxy-2,6-dimethylphenyl)-2-methyl-indazole-6-carboxamide (60 mg, 0.194 mmol, 1 eq) in DCM (1 mL) was added BBr₃ (0.1 mL). The mixture was stirred at 20° C. for 1 hour. The mixture was dropwise added into water (50 mL) and adjusted pH to 7 with aq.NaHCO₃. The mixture was extracted with DCM (30 mL×3), washed with aq. saturated NaCl (20 mL×3). The organic phase was dried over anhyd. Na₂SO₄, filtered and concentrated. The crude product was purified by RP-HPLC([water(NH₄HCO₃)-ACN]; B %: 2%-42%, 36 min) to give 4-(3-hydroxy-2,6-dimethylphenyl)-2-methyl-indazole-6-carboxamide (12.2 mg, 99% purity). LC-MS m/z 296 (M+H⁺). ¹H NMR: (400 MHz, DMSO-d₆) δ=9.16 (s, 1H), 8.18 (s, 1H), 8.02 (s, 1H), 7.92 (s, 1H), 7.33 (s, 1H), 7.24 (d, 1H), 6.94 (d, 1H), 6.77 (d, 1H), 4.13 (s, 3H), 1.80 (s, 3H), 1.72 (s, 3H).

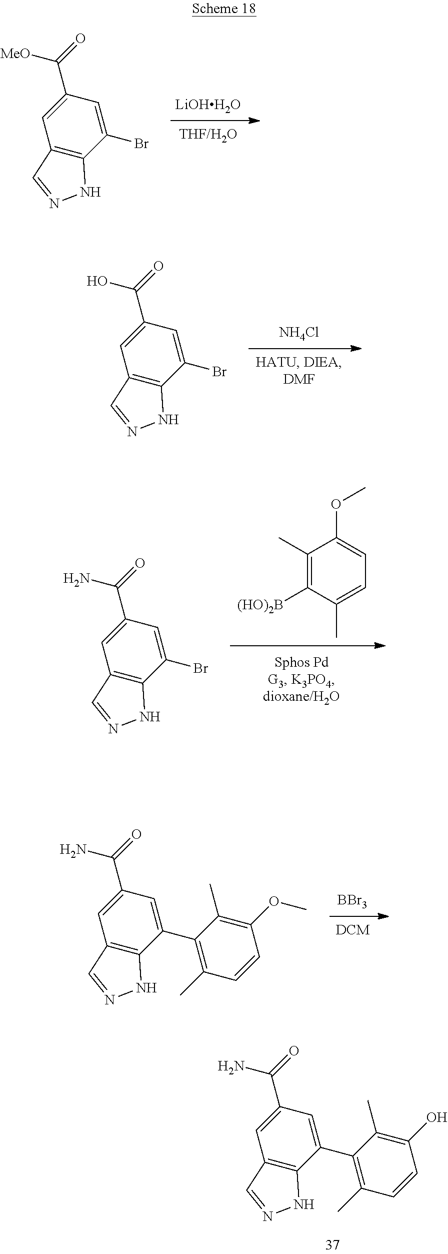

### Example 31. 7-(3-Hydroxy-2,6-dimethylphenyl)-1H-indazole-5-carboxamide (37)

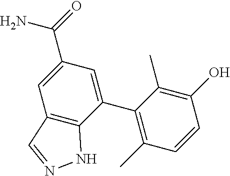 [SMILES: Cc1ccc(O)c(C)c1-c1cc(C(N)=O)cc2cn[nH]c12]

### Step 1: Synthesis of 7-bromo-1H-indazole-5-carboxylic acid

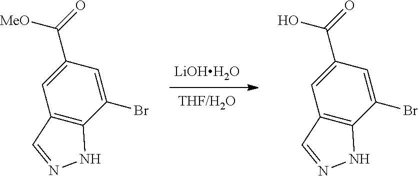 [SMILES: COC(=O)c1cc(Br)c2[nH]ncc2c1 ; "LiOH·H2O" ; "THF/H2O" ; O=C(O)c1cc(Br)c2[nH]ncc2c1]

To a solution of methyl 7-bromo-1H-indazole-5-carboxylate (350 mg, 1.37 mmol, 1 eq) in THF (5 mL) and H₂O (5 mL) was added LiOH·H₂O (173 mg, 4.12 mmol, 3 eq). The mixture was stirred at 25° C. for 16 hours. The reaction was concentrated to remove THF. The residue was adjusted to pH 6 with aq. HCl (2 M). The mixture was filtered to give the filtrate cake, which was concentrated directly to give 7-bromo-1H-indazole-5-carboxylic acid (358 mg, 89% yield, 82% purity). LC-MS m/z 241 (M+H⁺).

### Step 2: Synthesis of 7-bromo-1H-indazole-5-carboxamide

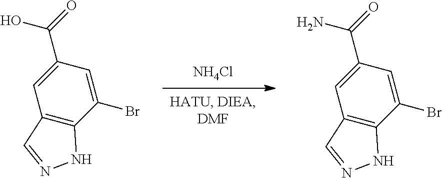 [SMILES: O=C(O)c1cc(Br)c2[nH]ncc2c1 ; "HATU, DIEA,DMF" ; "NH4Cl" ; NC(=O)c1cc(Br)c2[nH]ncc2c1]

To a solution of 7-bromo-1H-indazole-5-carboxylic acid (358 mg, 1.22 mmol, 82% purity, 1 eq) in DMF (4 mL) was added HATU (556 mg, 1.46 mmol, 1.2 eq), DIEA (472 mg, 3.65 mmol, 0.6 mL, 3 eq) and NH₄Cl (98 mg, 1.83 mmol, 1.5 eq). The mixture was stirred at 25° C. for 16 hours. The reaction mixture was diluted with water (30 mL) and extracted with EtOAc (20 mL×3). The combined organic layers were washed with aq. saturated NaCl (20 mL×3), dried over anhyd. Na₂SO₄, filtered and concentrated to give 7-bromo-1H-indazole-5-carboxamide (300 mg, 91% yield, 89% purity), which was used into the next step without further purification. LC-MS m/z 240 (M+H⁺).

### Step 3: Synthesis of 7-(3-methoxy-2,6-dimethylphenyl)-1H-indazole-5-carboxamide

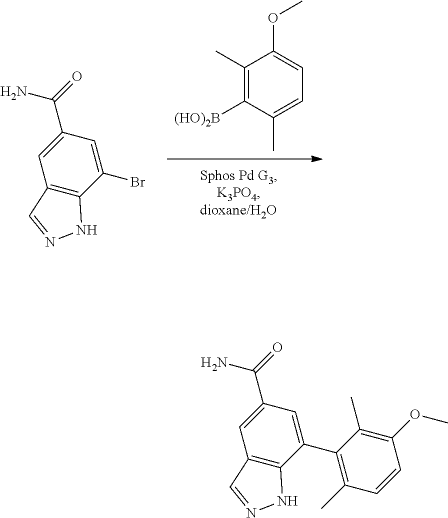

To a solution of 7-bromo-1H-indazole-5-carboxamide (300 mg, 1.25 mmol, 1 eq) and (3-methoxy-2,6-dimethylphenyl)boronic acid (337 mg, 1.87 mmol, 1.5 eq) in dioxane (5 mL) and H₂O (1.5 mL) was added SPhos Pd G3 (97 mg, 0.12 mmol, 0.1 eq) and K₃PO₄ (796 mg, 3.75 mmol, 3 eq). The mixture was stirred at 100° C. under N₂ for 12 hours. The reaction mixture was diluted with water (50 mL) and extracted with DCM (40 mL×3). The combined organic layers were washed with aq. saturated NaCl (30 mL×3), dried over anhyd. Na₂SO₄, filtered and concentrated to give a residue. The residue was purified by column chromatography (SiO₂, 0-7% MeOH in DCM) to give 7-(3-methoxy-2,6-dimethylphenyl)-1H-indazole-5-carboxamide (90 mg, 24% yield). LC-MS m/z 296 (M+H⁻).

### Step 4: Synthesis of 7-(3-hydroxy-2,6-dimethylphenyl)-1H-indazole-5-carboxamide (compound 37)

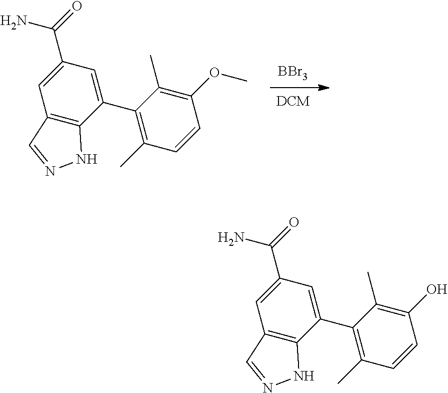

To a solution of 7-(3-methoxy-2,6-dimethylphenyl)-1H-indazole-5-carboxamide (80 mg, 0.27 mmol, 1 eq) in DCM (1 mL) was added BBr₃ (0.05 mL, 2 eq) at 0° C. After stirring at 0° C. for 1 hour, the reaction mixture was quenched with water (20 mL) at 0° C., extracted with DCM (20 mL×3). The combined organic layers were washed with aq. saturated NaCl (15 mL×2), dried over anhyd. Na₂SO₄, filtered and concentrated. The residue was purified by RP-HPLC (column: Welch Xtimate C18 150×30 mm×5 um; mobile phase: [water(FA)-ACN]; B %: 0%-36%, 14 min) to give 7-(3-hydroxy-2,6-dimethylphenyl)-1H-indazole-5-carboxamide (4.4 mg, 99.56% purity). LC-MS m/z 282 (M+H⁺). ¹H NMR: (400 MHz, DMSO-d₆) δ=12.92 (s, 1H), 9.20 (s, 1H), 8.35 (s, 1H), 8.22 (s, 1H), 7.96 (s, 1H), 7.57 (d, 1H), 7.24 (s, 1H), 6.99 (d, 1H), 6.82 (d, 1H), 1.79 (s, 3H), 1.72 (s, 3H).

### Example 32. 7-(3-Hydroxy-2,6-dimethylphenyl)-N-methyl-1H-indazole-5-carboxamide (38)

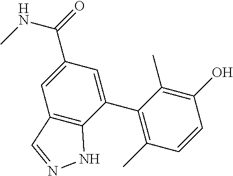 [SMILES: CNC(=O)c1cc(-c2c(C)ccc(O)c2C)c2[nH]ncc2c1]

This was made in the same manner as Example 31 using methylamine in step 2. LC-MS m/z 296 (M+H⁺). ¹H NMR: (400 MHz, DMSO-d₆) 5=12.99 (s, 1H), 9.27 (s, 1H), 8.51 (s, 1H), 8.36 (s, 1H), 8.28 (s, 1H), 7.58 (s, 1H), 7.05 (d, 1H), 6.87 (d, 1H), 2.84 (d, 3H), 1.84 (s, 3H), 1.77 (s, 3H).

### Example 33. 7-(3-Hydroxy-2,6-dimethylphenyl)-N,N-dimethyl-1H-indazole-5-carboxamide (39)

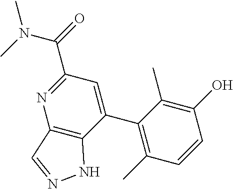 [SMILES: Cc1ccc(O)c(C)c1-c1cc(C(=O)N(C)C)nc2cn[nH]c12]

This was made in the same manner as Example 31 using dimethylamine in step 2. LC-MS m/z 310 (M+H⁺). ¹H NMR: (400 MHz, DMSO-d₆) δ=12.93-12.77 (m, 1H), 9.27-9.15 (m, 1H), 8.17 (s, 1H), 7.83 (s, 1H), 7.02 (s, 1H), 6.98 (d, 1H), 6.81 (d, 1H), 2.99 (s, 6H), 1.80 (s, 3H), 1.73 (s, 3H).

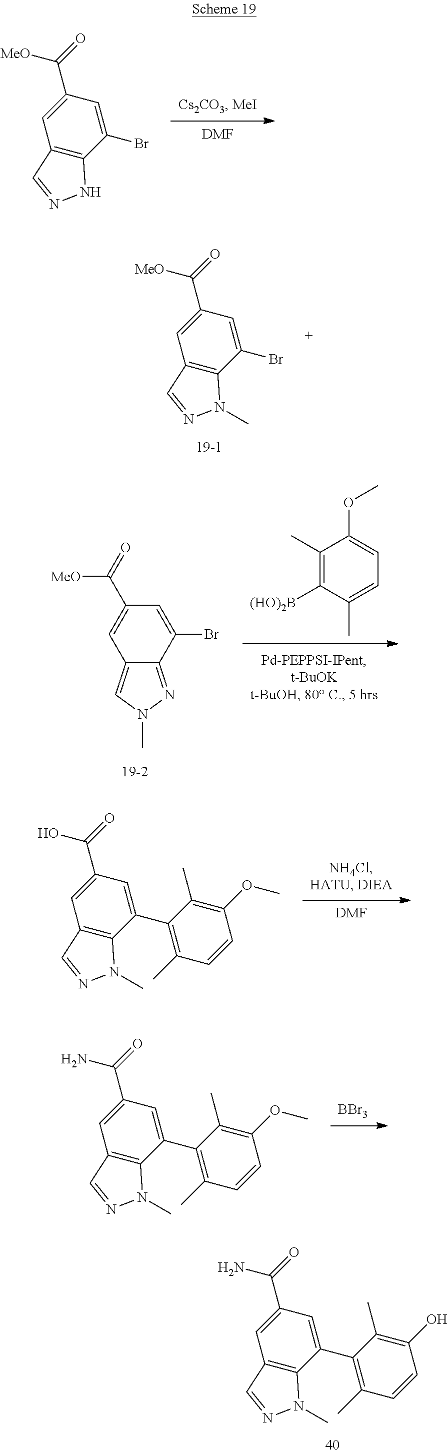

### Example 34. 7-(3-Hydroxy-2,6-dimethylphenyl)-1-methyl-1H-indazole-5-carboxamide (40)

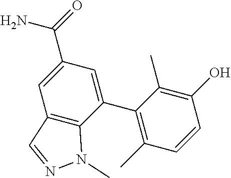 [SMILES: Cc1ccc(O)c(C)c1-c1cc(C(N)=O)cc2cnn(C)c12]

### Step 1: Synthesis of methyl 7-bromo-1-methyl-indazole-5-carboxylate methyl 7-bromo-2-methyl-indazole-5-carboxylate

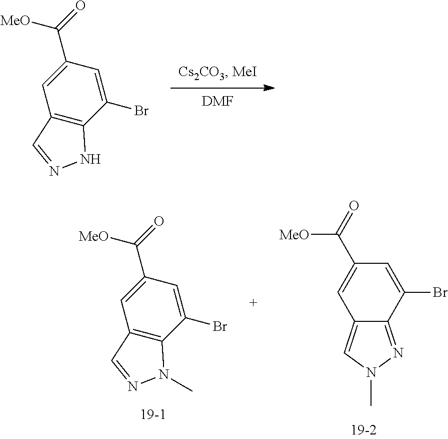

To a solution of methyl 7-bromo-1H-indazole-5-carboxylate (500 mg, 2 mmol, 1 eq) in DMF (5 mL) was added Cs₂CO₃ (2 g, 6 mmol, 3 eq) and MeI (334 mg, 2.4 mmol, 1.2 eq). The mixture was stirred at 25° C. for 16 hours. The reaction mixture was diluted with water (20 mL) and extracted with EtOAc (100 mL×3). The combined organic phase was washed with aq. saturated NaCl (100 mL×2), dried with anhydrous Na₂SO₄, filtered, and concentrated to a residue. The residue was purified by column chromatography (SiO₂, 0-20% ethyl acetate in petroleum ether) to afford methyl 7-bromo-1-methyl-indazole-5-carboxylate (19-1. 300 mg, 56.63% yield) and methyl 7-bromo-2-methyl-indazole-5-carboxylate (19-2, 100 mg, 18.96% yield). 19-1: LC-MS m/z 271 (M+H⁻). ¹H NMR: (400 MHz, DMSO-d₆) δ=8.47 (d, 1H), 8.33 (s, 1H), 8.08 (d, 1H), 4.35 (s, 3H), 3.87 (s, 3H), 19-2: LC-MS m/z 271 (M+H⁺). ¹H NMR: (400 MHz, DMSO-d₆) δ=8.75 (s, 1H), 8.51 (s, 1H), 7.94 (s, 1H), 4.24 (s, 3H), 3.86 (s, 3H).

### Step 2: Synthesis of 7-(3-methoxy-2,6-dimethylphenyl)-1-methyl-indazole-5-carboxylic acid

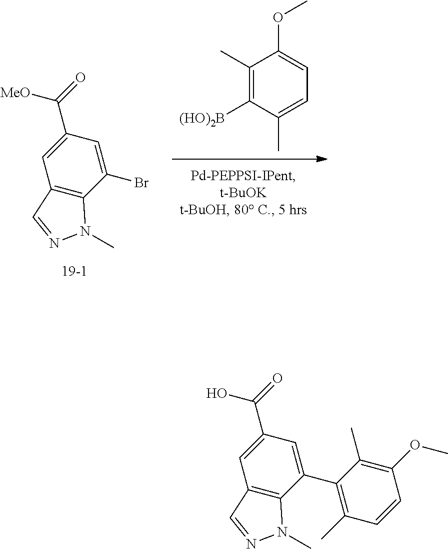

To a solution of methyl 4-bromo-1-methyl-indazole-6-carboxylate (19-1. 200 mg, 0.7 mmol, 1 eq) and (3-methoxy-2,6-dimethylphenyl)boronic acid (200 mg, 1.1 mmol, 1.5 eq) in t-BuOH (5 mL) was added t-BuOK (333 mg, 2.8 mmol, 4 eq) and 1,3-bis[2,6-bis(1-ethylpropyl)phenyl]-2H-imidazole; 3-chloropyridine; dichloropalladium (59 mg, 0.07 mmol, 0.1 eq). The mixture was stirred at 80° C. for 5 hours. The mixture was concentrated to give a residue, which was purified by RP-HPLC column: (C18 75×30 mm×3 um; mobile phase: [water-MeOH]; B %: 0%-100%) to give 7-(3-methoxy-2,6-dimethylphenyl)-1-methyl-indazole-5-carboxylic acid (50 mg). LC-MS m/z 311 (M+H⁺).

### Step 3: Synthesis of 7-(3-methoxy-2,6-dimethylphenyl)-1-methyl-indazole-5-carboxamide

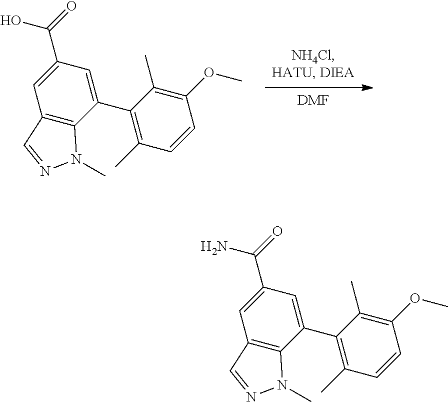

To a solution of 7-(3-methoxy-2,6-dimethylphenyl)-1-methyl-indazole-5-carboxylic acid (60 mg, 0.2 mmol, 1 eq) in DMF (2 mL) was added NH₄Cl (21 mg, 0.4 mmol, 2 eq), HATU (110 mg, 0.3 mmol, 1.5 eq) and DIEA (75 mg, 0.6 mmol, 3 eq). After stirring at 25° C. for 16 hours, the mixture was diluted with water (20 mL) and extracted with DCM (30 mL×3). The combined organic layers were washed with aq. saturated NaCl (20 mL×2), dried over anhydrous Na₂SO₄, filtered, and concentrated to afford 7-(3-methoxy-2,6-dimethylphenyl)-1-methyl-indazole-5-carboxamide (50 mg, crude). LC-MS m/z 310 (M+H⁺).

### Step 4: Synthesis of 7-(3-hydroxy-2,6-dimethylphenyl)-1-methyl-indazole-5-carboxamide (compound 40)

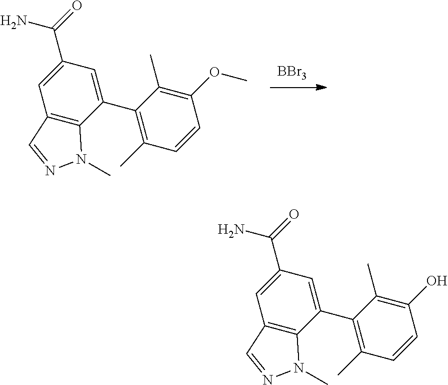

To a solution of 7-(3-methoxy-2,6-dimethylphenyl)-1-methyl-indazole-5-carboxamide (50 mg, crude) in DCM (1 mL) was added BBr₃ (0.1 mL, 5 eq) at 0° C. The mixture was stirred at 25° C. for 2 hours. The reaction mixture was dropwise added to water (20 mL) at 20° C. The reaction mixture was adjusted pH to 7 by aq. saturated NaHCO₃ at 0° C. and extracted with DCM (50 mL×3). The combined organic layers were washed with aq. saturated NaCl (100 mL×2), dried over anhyd. Na₂SO₄, filtered and concentrated. The residue was purified by RP-HPLC column: (C18 75×30 mm×3 um; mobile phase: [water(NH₃H₂O+NH₄HCO₃)-ACN]; B %: 4%-44%, 30 min) to afford 7-(3-hydroxy-2,6-dimethylphenyl)-1-methyl-indazole-5-carboxamide (11.8 mg, 0.04 mmol). LC-MS m/z 296 (M+H⁺). ¹H NMR: (400 MHz, DMSO-d₆) δ=9.36 (s, 1H), 8.35 (d, H), 8.22 (s, 1H), 8.05 (s, 1H), 7.56 (d, 1H), 7.31 (s, 1H), 7.00 (d, 1H), 6.83 (d, 1H), 3.43 (s, 3H), 1.77 (s, 3H), 1.72 (s, 3H).

### Example 35. 7-(3-Hydroxy-2,6-dimethylphenyl)-2-methyl-2H-indazole-5-carboxamide (41)

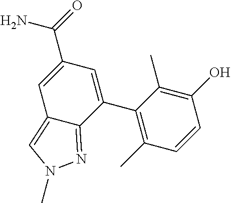 [SMILES: Cc1ccc(O)c(C)c1-c1cc(C(N)=O)cc2cn(C)nc12]

This compound was synthesized in the same manner as Example 34 using 19-2 in step 2. LC-MS m/z 296 (M+H⁺). ¹H NMR: (400 MHz, DMSO) δ=9.07 (s, 1H), 8.53 (s, 1H), 8.32 (d, 1H), 8.00-7.80 (m, 1H), 7.42 (s, 1H), 7.30 (s, 1H), 6.88 (d, 1H), 6.75 (d, 1H), 4.11 (s, 3H), 1.75 (m, 3H), 1.66 (s, 3H).

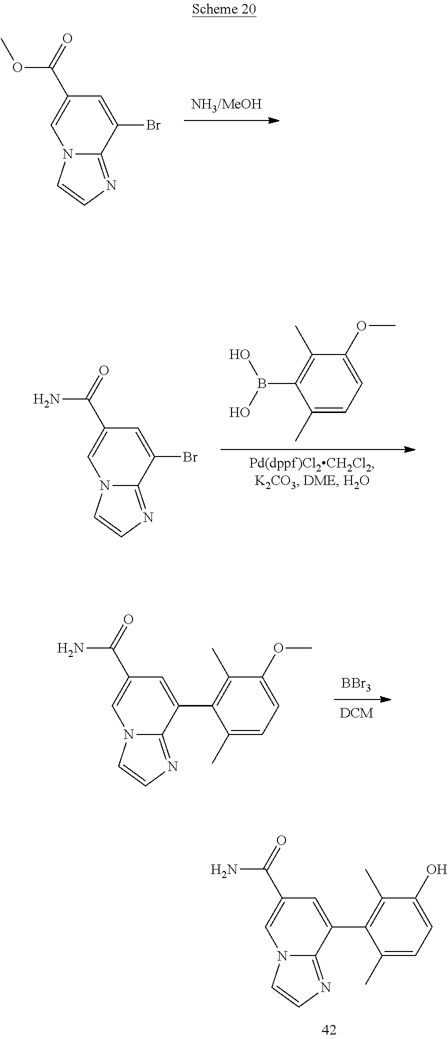

### Example 36. 8-(3-Hydroxy-2,6-dimethylphenyl)imidazo[1,2-a]pyridine-6-carboxamide (42)

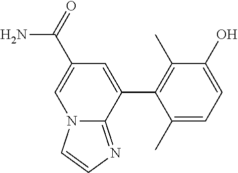 [SMILES: Cc1ccc(O)c(C)c1-c1cc(C(N)=O)cn2ccnc12]

### Step 1: Synthesis of 8-bromoimidazo[1,2-a]pyridine-6-carboxamide

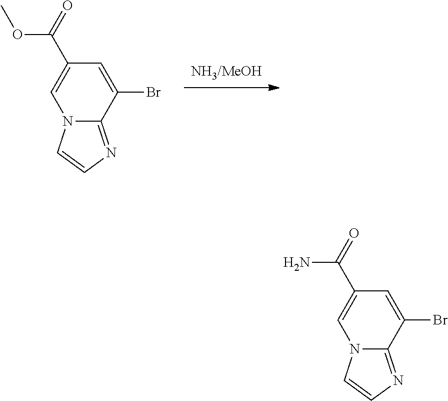

To a solution of methyl 8-bromoimidazo[1,2-a]pyridine-6-carboxylate (500 mg, 1.96 mmol, 1 eq) in NH₃/MeOH (15 mL, 7M). The mixture was stirred at 60° C. for 6 hours. The reaction mixture was concentrated under vacuum. The crude product was triturated with MTBE (15 mL) at 25° C. to give 8-bromoimidazo[1,2-a]pyridine-6-carboxamide (442 mg, 85% yield, 90% purity). LC-MS m/z 240 (M+H⁺).

### Step 2: Synthesis of 8-(3-methoxy-2,6-dimethylphenyl)imidazo[1,2-a]pyridine-6-carboxamide

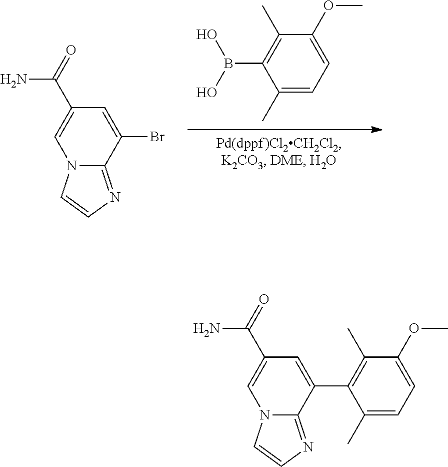

To a solution of 8-bromoimidazo[1,2-a]pyridine-6-carboxamide (300 mg, 1.25 mmol, 1 eq) and (3-methoxy-2,6-dimethylphenyl)boronic acid (289 mg, 1.62 mmol, 1.3 eq) in DME (10.5 mL) and H₂O (3.5 mL) was added K₂CO₃ (1.73 g, 12.50 mmol, 10 eq) and Pd(dppf)Cl₂·CH₂Cl₂ (102 mg, 0.13 mmol, 0.1 eq). The mixture was degassed with N₂ and stirred at 120° C. for 3 hours under microwave irradiation. The mixture was diluted with H₂O (20 mL) and EtOAc (30 mL×3). The combined organic layers were washed with aq. saturated NaCl (30 mL×3), dried over anhyd. Na₂SO₄, filtered and concentrated under vacuum. The crude product was purified by column chromatography (SiO₂, 0~6% MeOH in DCM) to give 8-(3-methoxy-2,6-dimethylphenyl)imidazo[1,2-a]pyridine-6-carboxamide (75 mg, 15% yield, 74% purity). LC-MS m/z 296 (M+H⁺).

### Step 3: Synthesis of 8-(3-hydroxy-2,6-dimethylphenyl)imidazo[1,2-a]pyridine-6-carboxamide (compound 42)

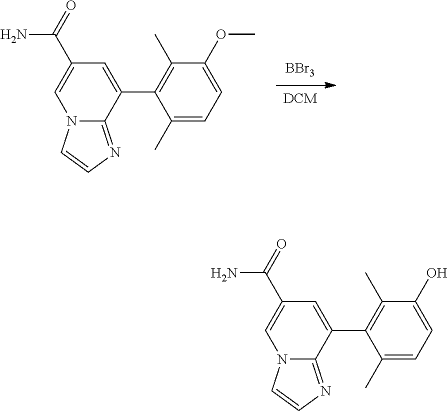

To a solution of 8-(3-methoxy-2,6-dimethylphenyl)imidazo[1,2-a]pyridine-6-carboxamide (55 mg, 0.14 mmol, 74% purity, 1 eq) in DCM (2 mL) was added BBr₃ (0.1 mL, 0.41 mmol, 3 eq) at 0° C. The mixture was stirred at 25° C. for 1 hour, then added dropwise into water (5 mL) at 20° C. The reaction mixture was adjusted to pH 7 by adding aq. saturated NaHCO₃ at 0° C. The mixture was extracted with DCM (10 mL×3). The combined organic layers were washed with aq. saturated NaCl (10 mL×2), dried over anhyd. Na₂SO₄, filtered and concentrated under vacuum. The crude product was purified by RP-HPLC (column: Welch Xtimate C18 150×30 mm×5 um; mobile phase: [water(NH₃H₂O+NH₄HCO₃)-ACN]; B %: 0%-36%, 36 min) to give 8-(3-hydroxy-2,6-dimethylphenyl)imidazo[1,2-a]pyridine-6-carboxamide (1.5 mg, 4% yield, 99% purity). LC-MS m/z 282 (M+H⁺). ¹H NMR: (400 MHz, DMSO-d₆) δ=9.15 (m, 2H), 8.12-8.00 (d, 2H), 7.59-7.47 (m, 2H), 7.44 (s, 1H), 6.93 (d, 1H), 6.78 (d, 1H), 1.82 (s, 3H), 1.74 (s, 3H).

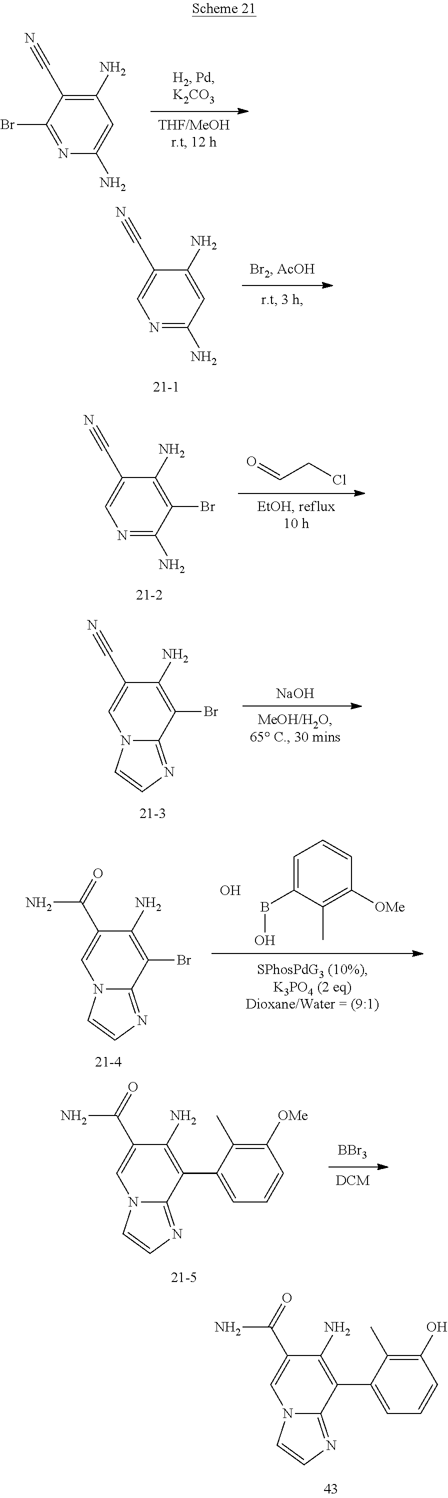

### Example 37. 7-Amino-8-(3-hydroxy-2-methylphenyl)imidazo[1,2-a]pyridine-6-carboxamide (43)

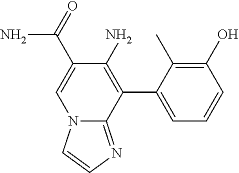 [SMILES: Cc1c(O)cccc1-c1c(N)c(C(N)=O)cn2ccnc12]

### Step 1: Synthesis of 4,6-diaminopyridine-3-carbonitrile

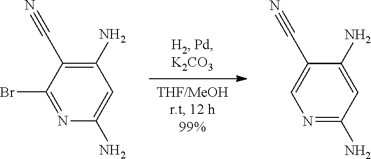

To a solution of 4,6-diamino-2-bromo-pyridine-3-carbonitrile (2 g, 1.0 equiv., 9.4 mmol) in THF was added palladium (20 mg, 0.02 equiv., 0.19 mmol). The mixture was stirred under hydrogen gas. After 2 hours, the reaction was stopped, and potassium acetate (921 mg, 1.0 equiv., 9.4 mmol) and methanol (5 mL) were added. The reaction vessel was recharged with hydrogen and stirred overnight. The solvent was removed under reduced pressure, and the residue was crystallized from isopropyl alcohol to afford the title compound 4,6-diaminopyridine-3-carbonitrile (1.24 g, 99% yield). LC-MS m/z 135 (M+H⁺). ¹H NMR (400 MHz, DMSO) δ 7.90 (d, J=2.6 Hz, 1H), 6.27 (d, J=7.0 Hz, 4H), 5.62 (d, J=2.4 Hz, 1H).

### Step 2: Synthesis of 4,6-diamino-5-bromo-pyridine-3-carbonitrile

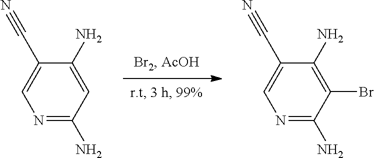

To the solution of 4,6-diaminopyridine-3-carbonitrile (21-1, 1.2 g, 1.0 equiv, 8.9 mmol) in acetic acid (20 mL) was added bromine (0.92 ml, 0.8 equiv. 7.5 mmol) at room temperature. The mixture was stirred for 3 hours at room temperature. The solvent was removed under reduced pressure to afford the title compound 4,6-diamino-5-bromo-pyridine-3-carbonitrile (1.9 g, crude). LC-MS m/z 215 (M+H⁺). ¹H NMR (400 MHz, DMSO) δ 8.40 (s, 1H), 7.71 (s, 4H).

### Step 3: Synthesis of 7-amino-8-bromoimidazo[1,2-a]pyridine-6-carbonitrile

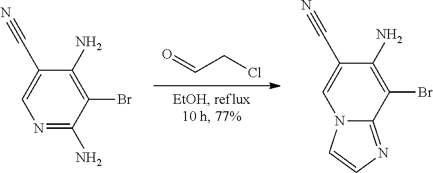

To a solution of 4,6-diamino-5-bromo-pyridine-3-carbonitrile (21-2, 0.77 g, 1.0 equiv., 3.6 mmol) in ethanol (10 mL) was added 2-chloroacetaldehyde (1.9 ml, 4.0 equiv., 14.5 mmol) 50% aqueous solution in one portion. The mixture was heated at reflux for 10 hours, cooled to room temperature, and the solvent was removed under reduced pressure. The resulting residue was dissolved in 100 ml of DCM, washed with NaHCO₃(aq.) and water, dried over anhydrous MgSO₄. The organic extracts were concentrated to give a crude product which was purified by column chromatography (0-20% methanol in DCM) to yield the title compound 7-amino-8-bromoimidazo[1,2-a]pyridine-6-carbonitrile (0.66 g, 77% yield). LC-MS m/z 239 (M+H⁻).

### Step 4: Synthesis of 7-amino-8-bromo-imidazo[1,2-a]pyridine-6-carboxamide

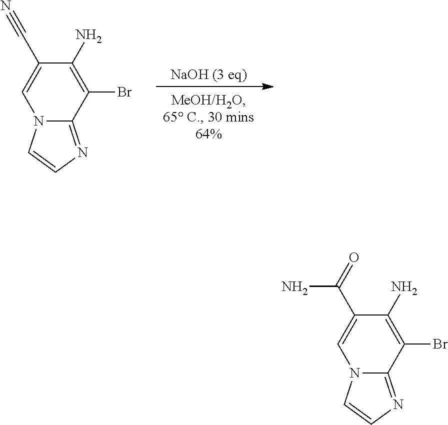 [SMILES: N#Cc1cn2ccnc2c(Br)c1N ; "MeOH/H2O,65° C., 30 mins64%" ; "NaOH (3 eq)" ; NC(=O)c1cn2ccnc2c(Br)c1N]

To a solution of 7-amino-8-bromoimidazo[1,2-a]pyridine-6-carbonitrile (21-3, 350 mg, 1.0 equiv. 1.5 mmol) in MeOH (5 ml) and water (5 mL) was added sodium hydroxide (177 mg, 3.0 equiv., 4.4 mmol). The mixture was heated to 65° C. for 30 mins, quenched by adding 1N HCl and adjusted to pH 7, and extracted with DCM (2×). The combined organic extract was washed with brine, dried with anhydrous MgSO₄ and concentrated. The residue was purified by column chromatography (0-20% methanol in DCM) to give the title compound 7-amino-8-bromo-imidazo[1,2-a]pyridine-6-carboxamide (240 mg, 64% yield). LC-MS m/z 257 (M+H⁺).

### Step 5: Synthesis of 7-amino-8-(3-methoxy-2-methylphenyl)imidazo[1,2-a]pyridine-6-carboxamide

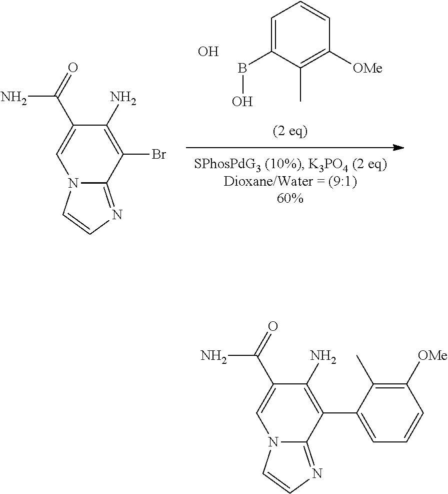

To a solution of 7-amino-8-bromo-imidazo[1,2-a]pyridine-6-carboxamide (21-4, 50 mg, 1.0 equiv., 0.2 mmol), (3-methoxy-2-methylphenyl)boronic acid (65 mg, 2.0 equiv., 0.4 mmol) and potassium phosphate (53 mg, 2.0 equiv., 0.4 mmol) in dioxane (2 ml) and water (0.2 ml) was added SPhos Pd G₃ (17.6 mg, 0.1 equiv., 0.02 mmol) under N₂ atmosphere. The reaction was stirred at 110° C. for 2 hours. The cooled reaction mixture was purified by RP-HPLC to give the title compound 7-amino-8-(3-methoxy-2-methylphenyl)imidazo[1,2-a]pyridine-6-carboxamide (35 mg, 60% yield). LC-MS m/z 297 (M+H⁻). ¹H NMR (400 MHz, DMSO) δ 8.73 (s, 1H), 8.24 (s, 2H), 7.57 (s, 1H), 7.19 (t, J=8.3 Hz, 1H), 7.14 (s, 1H), 6.94 (d, J=8.2 Hz, 1H), 6.70 (d, J=7.6 Hz, 1H), 5.46 (s, 2H), 3.77 (s, 3H), 1.78 (s, 3H).

### Step 6: Synthesis of 7-amino-8-(3-hydroxy-2-methylphenyl)imidazo[1,2-a]pyridine-6-carboxamide (compound 43)

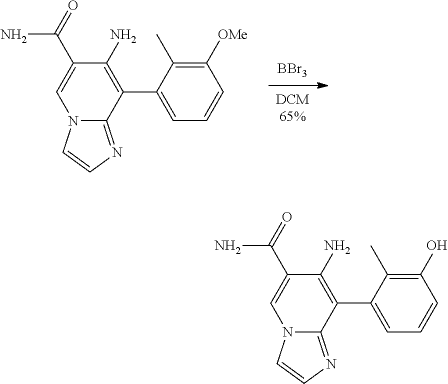

To a solution of 7-amino-8-(3-methoxy-2-methylphenyl)imidazo[1,2-a]pyridine-6-carboxamide (21-5, 100 mg, 0.34 mmol) in DCM (5 ml) was added boron tribromide (1.02 ml 1.0 M in DCM, 3.0 equiv., 1.02 mmol). The mixture was stirred at room temperature for 2 hours. The reaction was quenched by adding 5% NaHCO₃, extracted with DCM, concentrated, and then purified by RP-HPLC to give the title compound 7-amino-8-(3-hydroxy-2-methylphenyl)imidazo[1,2-a]pyridine-6-carboxamide (43 mg, 65% yield). LC-MS m/z 283 (M+H⁺). ¹H NMR (400 MHz, DMSO) δ 12.58 (s, 1H), 9.71 (s, 1H), 9.04 (d, J=2.9 Hz, 1H), 8.37 (s, 1H), 8.04-7.87 (m, 2H), 7.69 (s, 1H), 7.22 (t, J=8.3 Hz, 1H), 7.00 (d, J=8.0 Hz, 1H), 6.84 (s, 1H), 6.70 (d, J=7.5 Hz, 1H), 1.86 (d, J=2.7 Hz, 3H).

### Example 38. (P)-7-Amino-8-(3-hydroxy-2-methylphenyl)imidazo[1,2-a]pyridine-6-carboxamide (44) and (M)-7-amino-8-(3-hydroxy-2-methylphenyl)imidazo[1,2-a]pyridine-6-carboxamide (45)

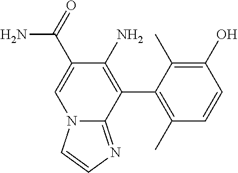 [SMILES: Cc1ccc(O)c(C)c1-c1c(N)c(C(N)=O)cn2ccnc12]

Chiral SFC separation of 7-amino-8-(3-methoxy-2-methylphenyl)imidazo[1,2-a]pyridine-6-carboxamide (compound 43, 50 mg, 0.15 mmol) (Instrument: Waters SFC Prep 150 Mgm; Column: Daicel Chiralpak OJ, 30×150 mm, 5 um; Conditions: Isocratic at 20% methanol with 80% CO₂; Flow Rate: 100 mL/min) provided Peak 1 (15 mg) and Peak 2 (33 mg).

Peak 1: retention time 4.01 min. LC-MS m/z 283 (M+H⁺). ¹H NMR (400 MHz, DMSO) δ 9.29 (s, 1H), 8.72 (s, 1H), 8.02 (s, 1H), 7.57 (s, 1H), 7.51 (s, 1H), 7.15 (s, 1H), 7.01 (t, J=7.6 Hz, 1H), 6.78 (d, J=7.9 Hz, 1H), 6.54 (d, J=7.5 Hz, 1H), 5.43 (s, 2H), 1.74 (d, J=2.6 Hz, 3H).

Peak 2: retention time 4.80 min. LC-MS m/z 283 (M+H⁺). ¹H NMR (400 MHz, DMSO) δ 9.50 (s, 1H), 8.90 (s, 1H), 8.21 (s, 1H), 7.76 (s, 2H), 7.41 (s, 1H), 7.14 (t, J=7.9 Hz, 1H), 6.91 (d, J=8.0 Hz, 1H), 6.65 (d, J=7.8 Hz, 1H), 6.03 (s, 2H), 1.83 (d, J=2.7 Hz, 4H).

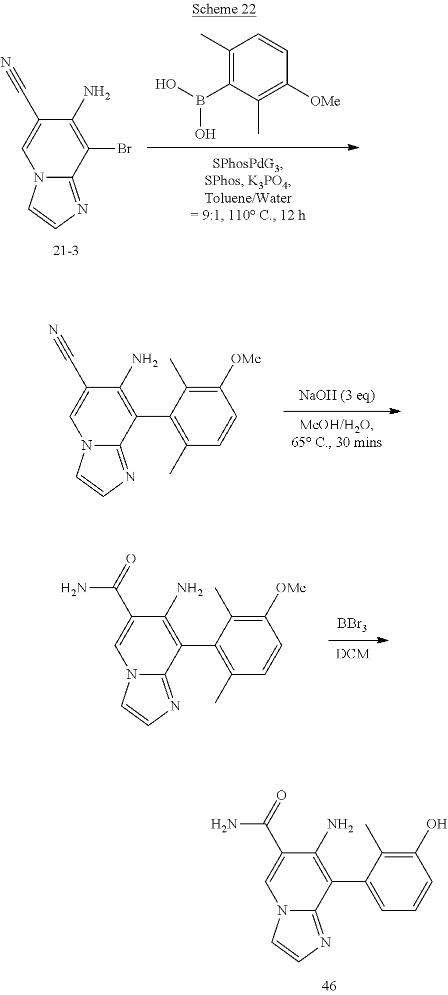

### Example 39. 7-Amino-8-(3-hydroxy-2,6-dimethylphenyl)imidazo[1,2-a]pyridine-6-carboxamide (46)

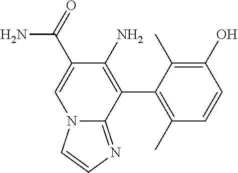 [SMILES: Cc1ccc(O)c(C)c1-c1c(N)c(C(N)=O)cn2ccnc12]

### Step 1: Synthesis of 7-amino-8-(3-methoxy-2,6-dimethylphenyl)imidazo[1,2-a]pyridine-6-carbonitrile

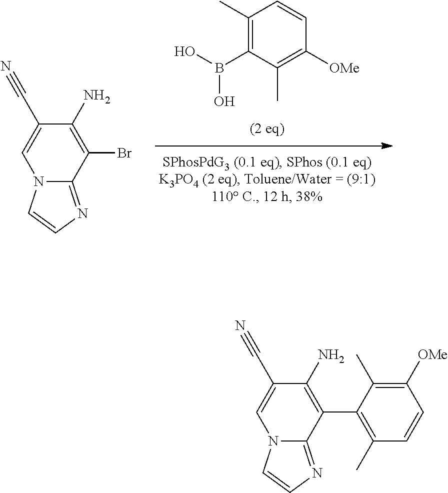 [SMILES: N#Cc1cn2ccnc2c(Br)c1N ; COc1ccc(C)c(B(O)O)c1C ; COc1ccc(C)c(-c2c(N)c(C#N)cn3ccnc23)c1C]

To a solution of 7-amino-8-bromo-imidazo[1,2-a]pyridine-6-carbonitrile (21-3, 150 mg, 1.0 equiv., 0.63 mmol), (3-methoxy-2,6-dimethylphenyl)boronic acid (227 mg, 2.0 equiv., 1.26 mmol) and potassium phosphate (170 mg, 2.0 equiv., 1.26 mmol) in toluene (2 ml) and water (0.2 ml) was added SPhos (26 mg, 0.1 equiv., 0.06 mmol) and SPhos Pd G₃ (57 mg, 0.1 equiv., 0.06 mmol) under N₂. The reaction was stirred at 110° C. for 12 hours. The cooled reaction mixture was quenched with water and extracted with DCM (2×). The combined organic extract was washed with brine, dried with anhydrous MgSO₄ and concentrated. The residue was purified by column chromatography (0-20% methanol in DCM) to give the title compound 7-amino-8-(3-methoxy-2,6-dimethylphenyl)imidazo[1,2-a]pyridine-6-carbonitrile (70 mg, 38% yield). LC-MS m/z 293 (M+H⁺).

### Step 2: Synthesis of 7-amino-8-(3-methoxy-2,6-dimethylphenyl)imidazo[1,2-a]pyridine-6-carboxamide

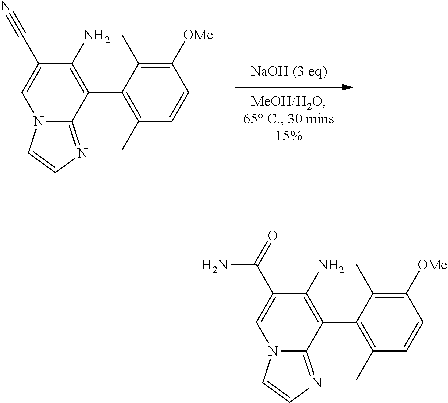 [SMILES: COc1ccc(C)c(-c2c(N)c(C#N)cn3ccnc23)c1C ; "MeOH/H2O,65° C., 30 mins15%" ; "NaOH (3 eq)" ; COc1ccc(C)c(-c2c(N)c(C(N)=O)cn3ccnc23)c1C]

To a solution of 7-amino-8-(3-methoxy-2,6-dimethylphenyl)imidazo[1,2-a]pyridine-6-carbonitrile (70 mg, 0.24 mmol %) in methanol (2 mL) and water (2 mL) was added sodium hydroxide (28 mg, 3.0 equiv., 0.72 mmol). The mixture was heated to 65° C. for 30 mins, cooled to room temperature, quenched by adding 1N HCl aq. to adjust pH ~7 and extracted with DCM (2×). The DCM extract was washed with brine, dried with anhydrous MgSO₄ and concentrated. The residue was purified by column chromatography (0-20% methanol in DCM) to give the title compound 7-amino-8-(3-methoxy-2,6-dimethylphenyl)imidazo[1,2-a]pyridine-6-carboxamide (11 mg, 15% yield). LC-MS m/z 311 (M+H⁻).

### Step 3: Synthesis of 7-amino-8-(3-hydroxy-2,6-dimethylphenyl)imidazo[1,2-a]pyridine-6-carboxamide (compound 46)

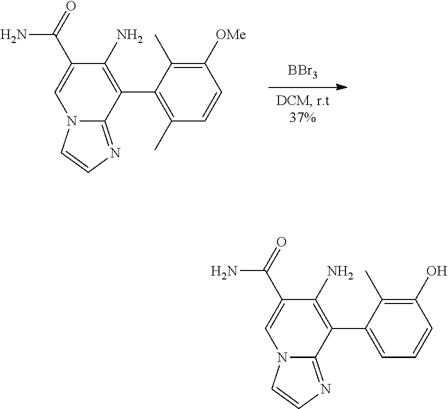 [SMILES: COc1ccc(C)c(-c2c(N)c(C(N)=O)cn3ccnc23)c1C ; "BBr3" ; "DCM, r.t37%" ; Cc1c(O)cccc1-c1c(N)c(C(N)=O)cn2ccnc12]

To a solution of 7-amino-8-(3-methoxy-2,6-dimethylphenyl)imidazo[1,2-a]pyridine-6-carboxamide (12 mg, 0.039 mmol) in DCM (0.5 ml) was added boron tribromide (0.12 ml 1.0 M in DCM, 3.0 equiv., 0.12 mmol). The mixture was stirred at room temperature for 2 hours, quenched by adding 5% NaHCO₃, and extracted with DCM. The combined organic extract was dried with anhydrous MgSO₄ and concentrated to give a crude material which was purified by RP-HPLC to yield the title compound of 7-amino-8-(3-hydroxy-2,6-dimethylphenyl)imidazo[1,2-a]pyridine-6-carboxamide (4 mg, 37% yield). LC-MS m/z 297 (M+H⁺). ¹H NMR (400 MHz, DMSO) δ 9.03 (s, 1H), 8.73 (s, 1H), 8.02 (s, 1H), 7.57 (s, 1H), 7.50 (s, 1H), 7.14 (s, 1H), 6.88 (d, J=8.1 Hz, 1H), 6.70 (d, J=8.1 Hz, 1H), 5.34 (s, 2H), 1.73 (s, 3H), 1.67 (s, 3H).

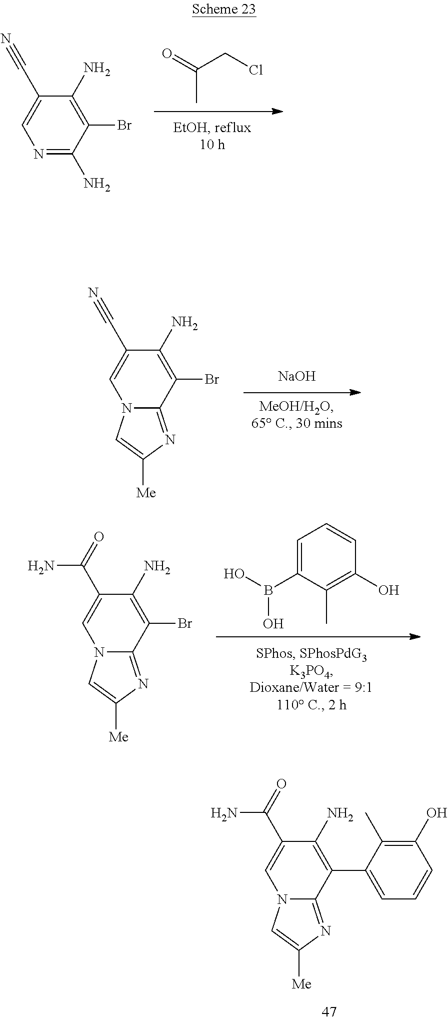

### Example 40. 7-Amino-8-(3-hydroxy-2-methylphenyl)-2-methylimidazo[1,2-a]pyridine-6-carboxamide (47)

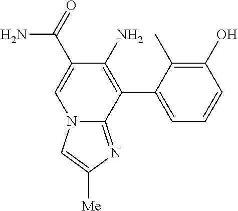 [SMILES: Cc1cn2cc(C(N)=O)c(N)c(-c3cccc(O)c3C)c2n1]

### Step 1: Synthesis of 7-amino-8-bromo-2-methyl-imidazo[1,2-a]pyridine-6-carbonitrile

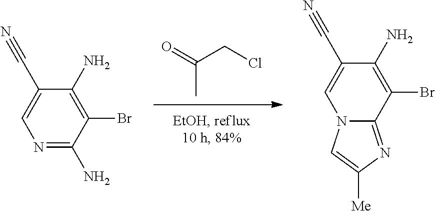

To a solution of 4,6-diamino-5-bromo-pyridine-3-carbonitrile (100 mg, 1.0 equiv., 0.47 mmol) in ethanol (2 mL) was added 1-chloropropan-2-one (0.15 ml, 4.0 equiv., 1.88 mmol) in one portion. The mixture was heated to reflux for 10 hours, cooled to room temperature, then concentrated in vacuo to give a residue. The residue was dissolved in 100 ml of DCM, washed with NaHCO₃(aq.) and water, dried over anhydrous MgSO₄ and concentrated. The resulting crude material was purified by column chromatography (0-20% methanol in DCM) to give the title compound 7-amino-8-bromo-2-methyl-imidazo[1,2-a]pyridine-6-carbonitrile (100 mg, 84% yield). LC-MS m/z 251 (M+H⁺).

### Step 2: Synthesis of 7-amino-8-bromo-2-methyl-imidazo[1,2-a]pyridine-6-carboxamide

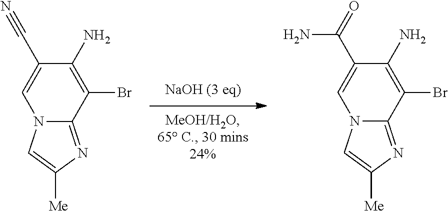 [SMILES: Cc1cn2cc(C#N)c(N)c(Br)c2n1 ; "MeOH/H2O,65° C., 30 mins24%" ; "NaOH (3 eq)" ; Cc1cn2cc(C(N)=O)c(N)c(Br)c2n1]

To a solution of 7-amino-8-bromo-2-methyl-imidazo[1,2-a]pyridine-6-carbonitrile (100 mg, 1.0 equiv. 0.4 mmol) in MeOH (0.5 mL) and water (0.5 mL) was added sodium hydroxide (48 mg, 3.0 equiv., 1.2 mmol). The mixture was heated to 65° C. for 30 mins, cooled to room temperature, quenched by adding 1N HCl to adjust pH ~7 and extracted with DCM (2×). The combined organic extract was washed with brine, dried with anhydrous MgSO₄ and concentrated. The residue was purified by column chromatography (0-20% methanol in DCM) to give the title compound 7-amino-8-bromo-2-methyl-imidazo[1,2-a]pyridine-6-carboxamide (26 mg, 24% yield). LC-MS m/z 271 (M+H⁺).

### Step 3: Synthesis of 7-amino-8-(3-hydroxy-2-methylphenyl)-2-methyl-imidazo[1,2-a]pyridine-6-carboxamide (compound 47)

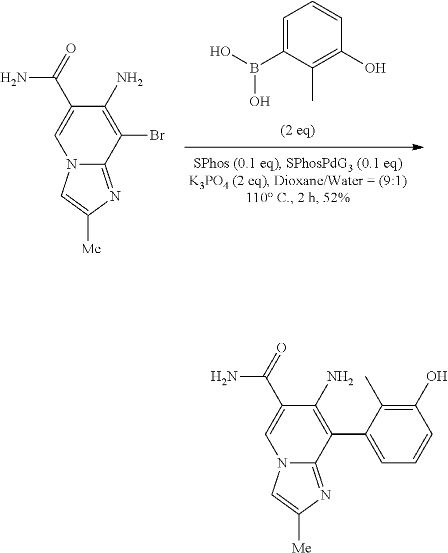 [SMILES: Cc1cn2cc(C(N)=O)c(N)c(Br)c2n1 ; Cc1c(O)cccc1B(O)O ; Cc1cn2cc(C(N)=O)c(N)c(-c3cccc(O)c3C)c2n1]

To a solution of 7-amino-8-bromo-2-methyl-imidazo[1,2-a]pyridine-6-carboxamide (26 mg, 1.0 equiv., 0.1 mmol), (3-hydroxy-2-methylphenyl)boronic acid (29.3 mg, 2.0 equiv., 0.2 mmol) and potassium phosphate (26 mg, 2.0 equiv., 0.2 mmol) in toluene (1 ml) and water (0.1 ml) was added SPhos (4 mg, 0.1 equiv., 0.01 mmol), SPhos Pd G₃ (8.8 mg, 0.1 equiv., 0.01 mmol) under N₂. The reaction was stirred at 110° C. for 2 hours, cooled to room temperature, quenched with water, extracted with DCM, dried with anhydrous MgSO₄ and concentrated. The crude material was purified by RP-HPLC to give the title compound 7-amino-8-(3-hydroxy-2-methylphenyl)-2-methyl-imidazo[1,2-a]pyridine-6-carboxamide (15 mg, 52% yield). LC-MS m/z 297 (M+H⁺). ¹H NMR (400 MHz, DMSO) δ 9.04 (s, 1H), 8.70 (s, 1H), 8.18 (s, 1H), 7.37 (s, 1H), 7.08 (t, J=8.1 Hz, 1H), 6.85 (d, J=7.9 Hz, 1H), 6.73 (t, J=8.1 Hz, 1H), 6.60 (d, J=7.5 Hz, 1H), 6.26 (s, 2H), 2.14 (s, 3H), 1.82 (s, 3H).

### Example 41. 7-Amino-8-(3-hydroxy-2-methylphenyl)-2,3-dimethylimidazo[1,2-a]pyridine-6-carboxamide (48)

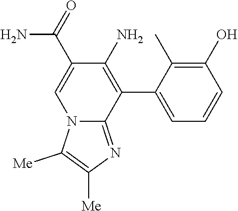 [SMILES: Cc1nc2c(-c3cccc(O)c3C)c(N)c(C(N)=O)cn2c1C]

This compound was synthetized in the same manner as Example 40, using 3-chlorobutan-2-one in step 1. LC-MS m/z 311 (M+H⁺). ¹H NMR (400 MHz, DMSO) δ 9.04 (s, 1H), 8.44 (s, 1H), 8.21 (s, 1H), 7.55 (s, 1H), 7.08 (t, J=7.5 Hz, 1H), 6.85 (d, J=8.0 Hz, 1H), 6.73 (t, J=7.7 Hz, 1H), 6.58 (d, J=7.6 Hz, 1H), 6.25 (d, J=7.7 Hz, 1H), 2.35 (s, 3H), 2.11 (s, 3H), 1.80 (s, 3H).

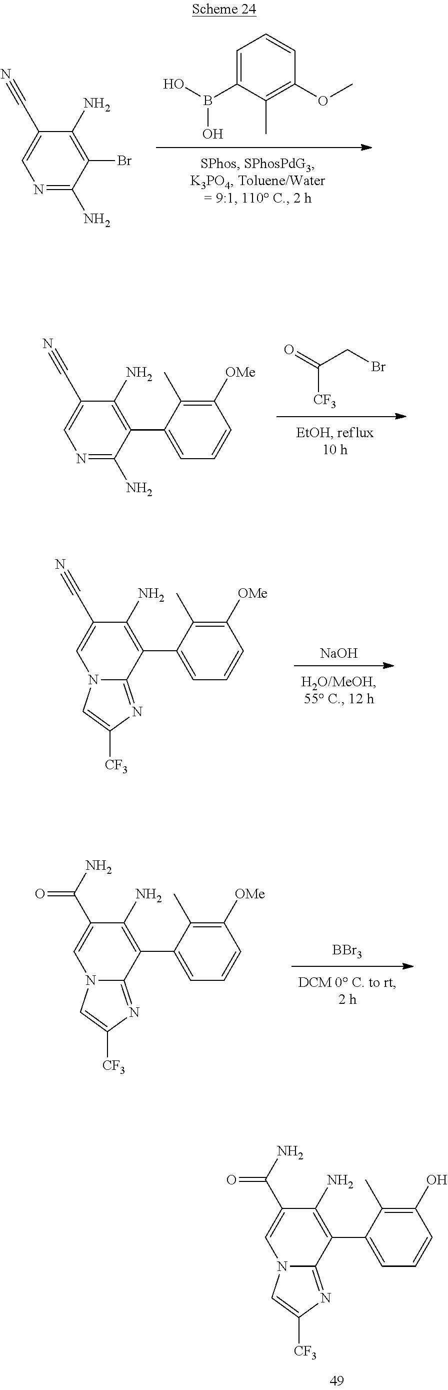

### Example 42. 7-Amino-5-(3-hydroxy-2-methylphenyl)imidazo[1,2-a]pyridine-6-carboxamide (49)

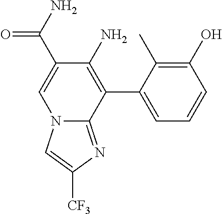 [SMILES: Cc1c(O)cccc1-c1c(N)c(C(N)=O)cn2cc(C(F)(F)F)nc12]

### Step 1: Synthesis of 4,6-diamino-5-(3-methoxy-2-methylphenyl)pyridine-3-carbonitrile

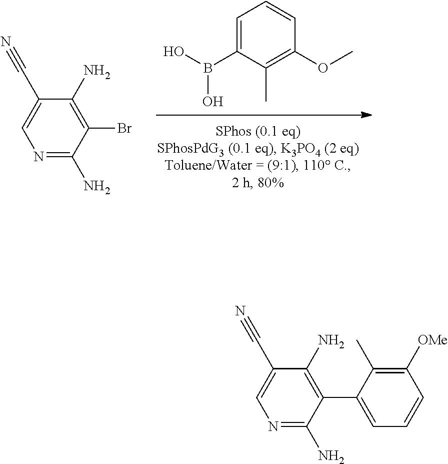 [SMILES: N#Cc1cnc(N)c(Br)c1N ; COc1cccc(B(O)O)c1C ; COc1cccc(-c2c(N)ncc(C#N)c2N)c1C]

To a solution of 4,6-diamino-5-bromo-pyridine-3-carbonitrile (800 mg, 1.0 equiv., 3.8 mmol), (3-methoxy-2-methylphenyl)boronic acid (935 mg, 1.5 equiv., 5.6 mmol) and potassium phosphate (1.0 g, 2.0 equiv., 7.6 mmol) in toluene (15 ml) and water (2 ml) was added SPhos (154 mg, 0.1 equiv., 0.38 mmol), SPhos Pd G3 (340 mg, 0.1 equiv., 0.38 mmol) under N₂. The reaction was stirred at 110° C. for 2 hours. The cooled reaction mixture was quenched with water and extracted with EtOAc(2×). The combined organic extract was washed with brine, dried with anhydrous MgSO₄ and concentrated. The residue was purified by column chromatography (0-100% EtOAc in hexane) to give the title compound 4,6-diamino-5-(3-methoxy-2-methylphenyl)pyridine-3-carbonitrile (760 mg, 80% yield). LC-MS m/z 255 (M+H⁺).

### Step 2: Synthesis of 7-amino-8-(3-methoxy-2-methylphenyl)-2-(trifluoromethyl)imidazo[1,2-a]pyridine-6-carbonitrile

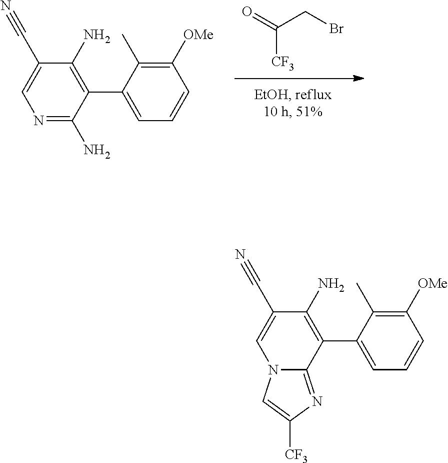 [SMILES: COc1cccc(-c2c(N)ncc(C#N)c2N)c1C ; O=C(CBr)C(F)(F)F ; COc1cccc(-c2c(N)c(C#N)cn3cc(C(F)(F)F)nc23)c1C]

To a solution of 4,6-diamino-5-(3-methoxy-2-methylphenyl)pyridine-3-carbonitrile (200 mg, 1.0 equiv., 0.79 mmol) in ethanol (2 mL) was added 3-bromo-1,1,1-trifluoro-propan-2-one (0.33 ml, 4.0 equiv., 1.87 mmol) in one portion. The mixture was heated to reflux for 10 hours, cooled to room temperature, and concentrated in vacuo. The resulting residue was dissolved in DCM (100 mL), washed with NaHCO₃(aq.) and water, dried with anhydrous MgSO₄ and concentrated to dryness. The crude product was purified by column chromatography (0-20% methanol in DCM) to give the title compound 7-amino-8-(3-methoxy-2-methylphenyl)-2-(trifluoromethyl)imidazo[1,2-a]pyridine-6-carbonitrile (140 mg, 51% yield). LC-MS m/z 347 (M+H⁺).

### Step 3: Synthesis of 7-amino-8-bromo-2,3-dimethyl-imidazo[1,2-a]pyridine-6-carboxamide

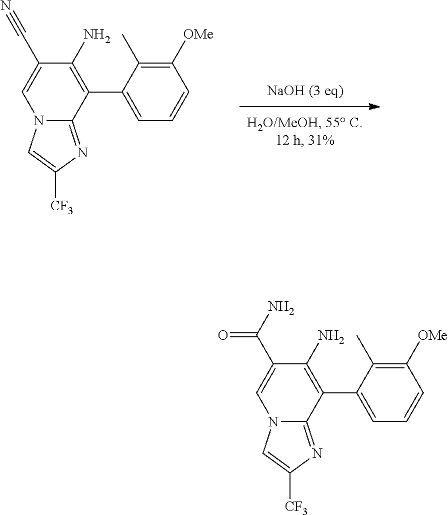 [SMILES: COc1cccc(-c2c(N)c(C#N)cn3cc(C(F)(F)F)nc23)c1C ; "NaOH (3 eq)" ; COc1cccc(-c2c(N)c(C(N)=O)cn3cc(C(F)(F)F)nc23)c1C]

To a solution of 7-amino-8-(3-methoxy-2-methylphenyl)-2-(trifluoromethyl)imidazo[1,2-a]pyridine-6-carbonitrile (140 mg, 1.0 equiv. 0.4 mmol) in MeOH (2 mL) and Water (2 mL) was added sodium hydroxide (48 mg, 3.0 equiv., 1.2 mmol). The mixture was heated at 55° C. for 12 hours, cooled to room temperature, adjusted to pH 7 by adding 1N HCl (aq.) and extracted with DCM (2×). The combined organic extract was washed with brine, dried with anhydrous MgSO₄ and concentrated. The residue was purified by column chromatography (0-20% methanol in DCM) to obtain the title compound 7-amino-8-(3-methoxy-2-methylphenyl)-2-(trifluoromethyl)imidazo[1,2-a]pyridine-6-carboxamide (60 mg, 31% yield). LC-MS m/z 365 (M+H⁺).

### Step 4: Synthesis of 7-amino-5-(3-hydroxy-2-methylphenyl)imidazo[1,2-a]pyridine-6-carboxamide (Example 49)

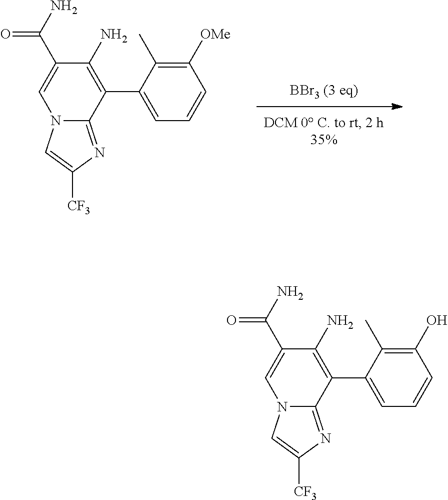 [SMILES: COc1cccc(-c2c(N)c(C(N)=O)cn3cc(C(F)(F)F)nc23)c1C ; "BBr3 (3 eq)" ; Cc1c(O)cccc1-c1c(N)c(C(N)=O)cn2cc(C(F)(F)F)nc12]

To a solution of 7-amino-8-(3-methoxy-2-methylphenyl)-2-(trifluoromethyl)imidazo[1,2-a]pyridine-6-carboxamide (60 mg, 0.16 mmol) in) in DCM (1 ml) was added boron tribromide (0.5 ml 1.0 M in DCM, 3.0 equiv., 0.48 mmol) at 0° C. The mixture was warmed to room temperature and stirred for 2 hours. The reaction was quenched with 5% NaHCO₃, extracted with DCM, and concentrated. The residue was purified by RP-HPLC to obtain the title compound 7-amino-8-(3-hydroxy-2-methylphenyl)-2-(trifluoromethyl)imidazo[1,2-a]pyridine-6-carboxamide (20 mg, 35% yield). LC-MS m/z 351 (M+H⁺). ¹H NMR (400 MHz, DMSO) δ 9.39 (s, 1H), 8.73 (s, 1H), 8.14 (s, 2H), 7.65 (s, 1H), 7.04 (t, J=7.8 Hz, 1H), 6.81 (d, J=8.3 Hz, 1H), 6.56 (d, J=7.4 Hz, 1H), 5.64 (s, 2H), 1.74 (s, 3H). ¹⁹F NMR (376 MHz, DMSO) δ −61.52.

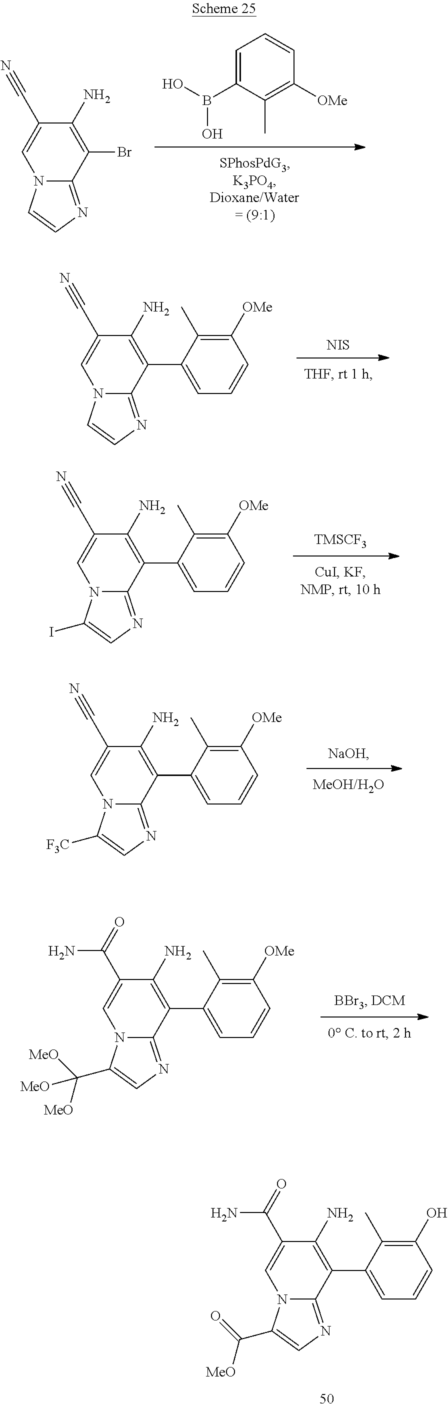

### Example 43. Methyl 7-amino-6-carbamoyl-8-(3-hydroxy-2-methylphenyl)imidazo[1,2-a]pyridine-3-carboxylate (50)

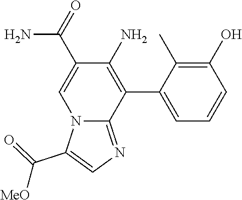 [SMILES: COC(=O)c1cnc2c(-c3cccc(O)c3C)c(N)c(C(N)=O)cn12]

### Step 1: Synthesis of 7-amino-8-(3-methoxy-2-methylphenyl)imidazo[1,2-a]pyridine-6-carbonitrile

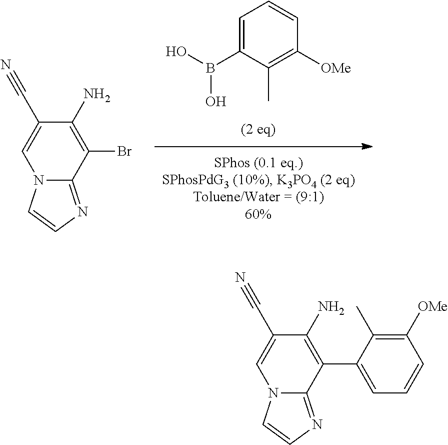 [SMILES: N#Cc1cn2ccnc2c(Br)c1N ; COc1cccc(B(O)O)c1C ; COc1cccc(-c2c(N)c(C#N)cn3ccnc23)c1C]

To a solution of 7-amino-5-bromoimidazo[1,2-a]pyridine-6-carbonitrile (150 mg, 1.0 equiv., 0.63 mmol), (3-methoxy-2-methylphenyl)boronic acid (210 mg, 2.0 equiv., 1.26 mmol) and potassium phosphate (181 mg, 2.0 equiv., 1.34 mmol) in toluene (2 ml) and water (0.2 ml) was added SPhos (26 mg, 0.1 equiv., 0.063 mmol) and SPhos Pd G₃ (57 mg, 0.1 equiv., 0.063 mmol) under N₂. The reaction was stirred at 110° C. for 2 hours. The cooled reaction mixture was quenched with water and extracted with DCM (2×). The combined organic extract was washed with brine, dried with anhydrous MgSO₄ and concentrated. The residue was purified by column chromatography (0-20% methanol in DCM) to give the title compound 7-amino-8-(3-methoxy-2-methylphenyl)imidazo[1,2-a]pyridine-6-carbonitrile (105 mg, 60% yield). LC-MS m/z 279 (M+H⁺).

### Step 2: Synthesis of 7-amino-3-iodo-8-(3-methoxy-2-methylphenyl)imidazo[1,2-a]pyridine-6-carbonitrile

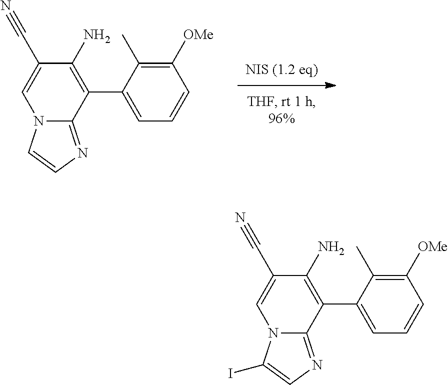 [SMILES: COc1cccc(-c2c(N)c(C#N)cn3ccnc23)c1C ; "NIS (1.2 eq)" ; COc1cccc(-c2c(N)c(C#N)cn3c(I)cnc23)c1C]

To a solution of 7-amino-8-(3-methoxy-2-methylphenyl)imidazo[1,2-a]pyridine-6-carbonitrile (100 mg, 0.36 mmol) in THF (2 mL) was added 1-iodopyrrolidine-2,5-dione (89 mg, 1.1 equiv., 0.4 mmol) portion-wise, under nitrogen at 0° C. The reaction was warmed to room temperature, stirred for 1 hour, and quenched with water (10 mL). The mixture was extracted with DCM (15 mL×3). The combined organic extract was washed with brine, dried with MgSO₄ and concentrated. The residue was purified by column chromatography (0-20% methanol in DCM) to yield the title compound 7-amino-3-iodo-8-(3-methoxy-2-methylphenyl)imidazo[1,2-a]pyridine-6-carbonitrile (140 mg, 96% yield). LC-MS m/z 405 (M+H⁺).

### Step 3: Synthesis of 7-amino-8-(3-methoxy-2-methylphenyl)-3-(trifluoromethyl)imidazo[1,2-a]pyridine-6-carbonitrile

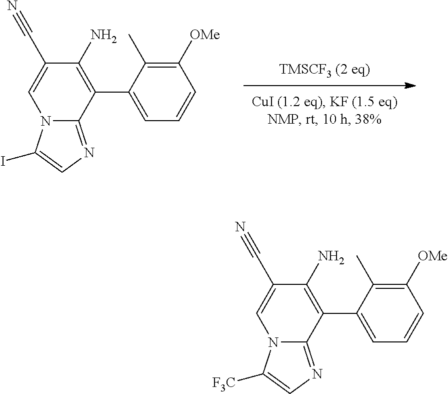 [SMILES: COc1cccc(-c2c(N)c(C#N)cn3c(I)cnc23)c1C ; "TMSCF3 (2 eq)" ; COc1cccc(-c2c(N)c(C#N)cn3c(C(F)(F)F)cnc23)c1C]

To a solution of 7-amino-3-iodo-8-(3-methoxy-2-methylphenyl)imidazo[1,2-a]pyridine-6-carbonitrile (140 mg, 0.35 mmol) in NMP (2 ml) was added copper (I) iodide (79 mg, 1.2 equiv., 0.42 mmol), potassium fluoride (31 mg, 1.5 equiv., 0.53 mmol), and trimethyl(trifluoromethyl)silane (0.1 ml, 2.0 equiv., 0.70 mmol) under nitrogen at room temperature. The mixture was stirred at room temperature for 10 hours, quenched with water and extracted with DCM (2×). The combined organic extract was washed with brine, dried with MgSO₄ and concentrated. The residue was purified by column chromatography (0-20% methanol in DCM) to get the title compound 7-amino-8-(3-methoxy-2-methylphenyl)-3-(trifluoromethyl)imidazo[1,2-a]pyridine-6-carbonitrile (120 mg, 38% yield). LC-MS m/z 347 (M+H⁺).

### Step 4: Synthesis of 7-amino-8-(3-methoxy-2-methylphenyl)-3-(trifluoromethyl)imidazo[1,2-a]pyridine-6-carbonitrile

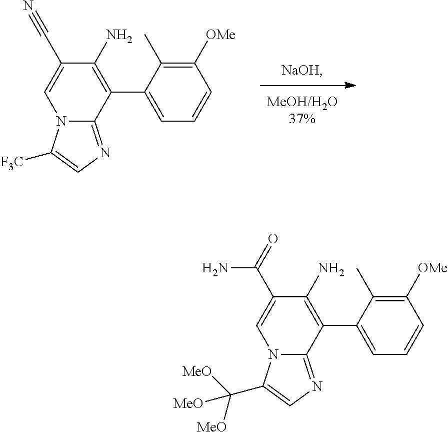 [SMILES: COc1cccc(-c2c(N)c(C#N)cn3c(C(F)(F)F)cnc23)c1C ; "NaOH," ; COc1cccc(-c2c(N)c(C(N)=O)cn3c(C(OC)(OC)OC)cnc23)c1C]

To a solution of 7-amino-8-(3-methoxy-2-methylphenyl)-3-(trifluoromethyl)imidazo[1,2-a]pyridine-6-carbonitrile (46 mg, 0.13 mmol) in MeOH (1 mL) and water (1 mL) was added sodium hydroxide (16 mg, 3.0 equiv., 0.39 mmol). The mixture was stirred at room temperature for 2 hours, quenched with 1N HCl (aq.) to pH 7 and extracted with DCM (2×). The combined DCM extract was washed with brine, dried with MgSO₄ and concentrated. The residue was purified by column chromatography (0-20% methanol in DCM) to obtain the title compound 7-amino-8-(3-methoxy-2-methylphenyl)-3-(trimethoxymethyl)imidazo[1,2-a]pyridine-6-carboxamide (20 mg, 37% yield). LC-MS m/z 401 (M+H⁺).

### Step 5: Synthesis of methyl 7-amino-6-carbamoyl-8-(3-hydroxy-2-methylphenyl)imidazo[1,2-a]pyridine-3-carboxylate (compound 50)

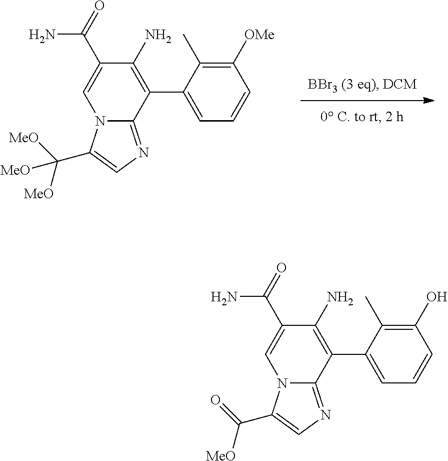 [SMILES: COc1cccc(-c2c(N)c(C(N)=O)cn3c(C(OC)(OC)OC)cnc23)c1C ; "BBr3 (3 eq), DCM" ; COC(=O)c1cnc2c(-c3cccc(O)c3C)c(N)c(C(N)=O)cn12]

To a solution of 7-amino-8-(3-methoxy-2-methylphenyl)-3-(trimethoxymethyl)imidazo[1,2-a]pyridine-6-carboxamide (20 mg, 0.05 mmol) in) in DCM (1 ml) was added boron tribromide (0.15 ml 1.0 M in DCM, 3.0 equiv., 0.15 mmol) at 0° C. The mixture was warmed to room temperature and stirred for 2 hours. The reaction was quenched with 5% NaHCO₃ (aq.), extracted with DCM, dried with MgSO₄ and concentrated. The residue was purified by RP-HPLC to give the title compound methyl 7-amino-6-carbamoyl-8-(3-hydroxy-2-methylphenyl)imidazo[1,2-a]pyridine-3-carboxylate (7 mg, 41% yield). LC-MS m/z 341 (M+H⁺). ¹H NMR (400 MHz, DMSO) δ 9.38 (s, 1H), 9.30 (s, 1H), 8.29 (s, 1H), 7.94 (s, 1H), 7.65 (s, 1H), 7.03 (t, J=7.9 Hz, 1H), 6.81 (d, J=8.2 Hz, 1H), 6.55 (d, J=7.8 Hz, 1H), 5.88 (s, 2H), 3.78 (s, 3H), 1.73 (s, 3H).

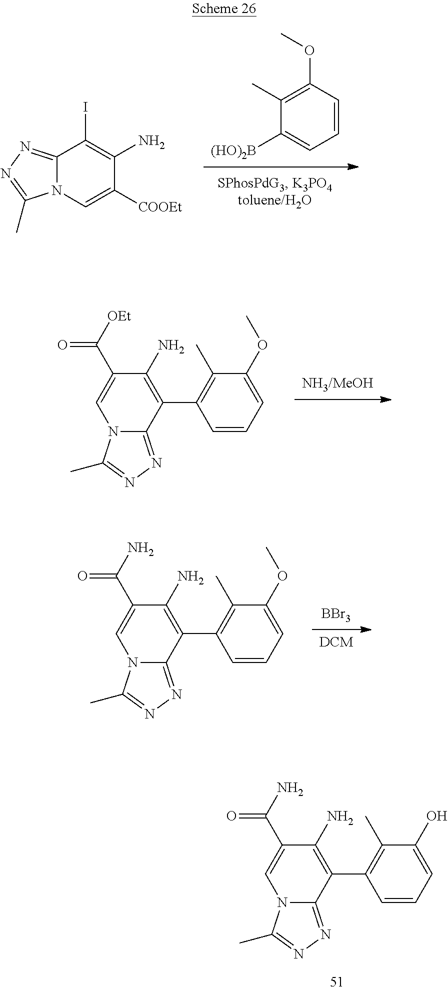

### Example 44. 7-Amino-8-(3-hydroxy-2-methylphenyl)-3-methyl-[1,2,4]triazolo[4,3-a]pyridine-6-carboxamide (51)

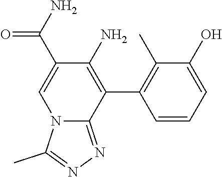 [SMILES: Cc1c(O)cccc1-c1c(N)c(C(N)=O)cn2c(C)nnc12]

### Step 1. Synthesis of 7-amino-8-(3-methoxy-2-methylphenyl)-3-methyl-[1,2,4]triazolo[4,3-a]pyridine-6-carboxylate

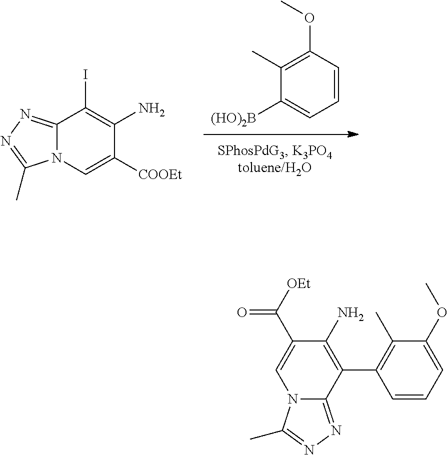 [SMILES: CCOC(=O)c1cn2c(C)nnc2c(I)c1N ; COc1cccc(B(O)O)c1C ; CCOC(=O)c1cn2c(C)nnc2c(-c2cccc(OC)c2C)c1N]

To a solution of ethyl 7-amino-8-iodo-3-methyl-[1,2,4]triazolo[4,3-a]pyridine-6-carboxylate (300 mg, 0.87 mmol, 1 eq) and (3-methoxy-2-methylphenyl)boronic acid (155 mg, 0.93 mmol, 1.1 eq) in toluene (5 mL) and H₂O (1 mL) was added SPhos Pd G₃ (68 mg, 0.1 mmol, 0.1 eq) and K₃PO₄ (370 mg, 1.74 mmol, 2 eq). The mixture was stirred at 100° C. under N₂ for 12 hours. The reaction mixture was concentrated to give a residue, which was diluted with water (30 mL), followed by extraction with EtOAc (50 mL×3). The combined organic phase was dried over anhyd. Na₂SO₄, filtered and concentrated under vacuum. The crude product was purified by flash silica gel chromatography (ISCO®; 4 g SepaFlash® Silica Flash Column, Eluent of 0~19% Ethyl acetate/Petroleum ether gradient a 18 m/min) to give ethyl 7-amino-8-(3-methoxy-2-methylphenyl)-3-methyl-[1.2.4]triazolo[4,3-a]pyridine-6-carboxylate (298 mg, 88% yield, 87.8% purity). LC-MS m/z 341 (M+H⁻).

### Step 2. Synthesis of 7-amino-8-(3-methoxy-2-methylphenyl)-3-methyl-[1,2,4]triazolo[4,3-a]pyridine-6-carboxamide

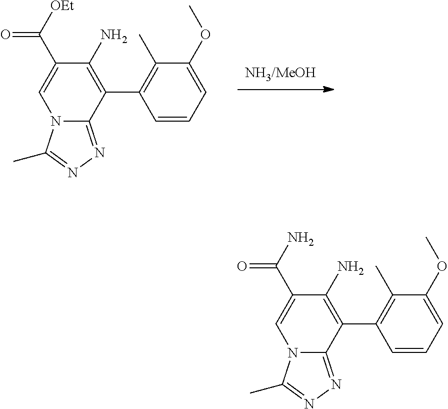 [SMILES: CCOC(=O)c1cn2c(C)nnc2c(-c2cccc(OC)c2C)c1N ; "NH3/MeOH" ; COc1cccc(-c2c(N)c(C(N)=O)cn3c(C)nnc23)c1C]

A solution of ethyl 7-amino-8-(3-methoxy-2-methylphenyl)-3-methyl-[1,2,4]triazolo[4,3-a]pyridine-6-carboxylate (298 mg, 0.88 mmol, 1 eq) in NH₃/MeOH (6 mL, 7M) was stirred at 60° C. for 5 hours. The reaction mixture was concentrated under vacuum to give 7-amino-8-(3-methoxy-2-methylphenyl)-3-methyl-[1,2,4]triazolo[4,3-a]pyridine-6-carboxamide (212 mg, crude). LC-MS m/z 312 (M+H⁺).

### Step 3. Synthesis of 7-amino-8-(3-hydroxy-2-methylphenyl)-3-methyl-[1,2,4]triazolo[4,3-a]pyridine-6-carboxamide (compound 51)

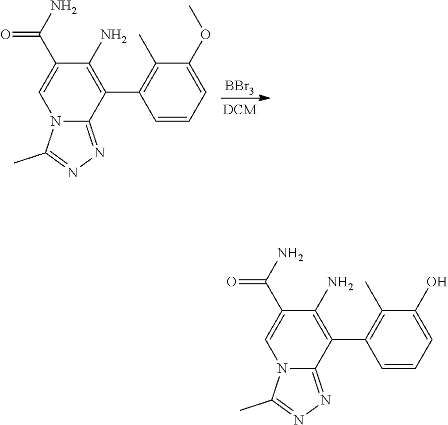

To a solution of 7-amino-8-(3-methoxy-2-methylphenyl)-3-methyl-[1,2,4]triazolo[4,3-a]pyridine-6-carboxamide (210 mg, 0.67 mmol, 1 eq) in DCM (3 mL) was added BBr₃ (0.5 mL, 7.7 eq) at 0° C. The mixture was stirred at 0° C. for 0.5 hour, then quenched by H₂O (20 mL). The mixture was extracted with DCM (10 mL×3). The organic phase was washed with aq. saturated NaCl, dried over anhyd. Na₂SO₄, filtered and concentrated. The crude product was purified by RP-HPLC (column: Phenomenex C18 75×30 mm×3 um; mobile phase: [water(HCl)-ACN]; B %: 0%-24%, 36 min) to give 7-amino-8-(3-hydroxy-2-methylphenyl)-3-methyl-[1,2,4]triazolo[4,3-a]pyridine-6-carboxamide (86 mg, 0.28 mmol, 98.58% purity). LC-MS m/z 298 (M+H⁺). ¹H NMR: (400 MHz, DMSO-d₆) δ=9.79 (s, 1H), 9.20 (s, 1H), 8.79 (s, 1H), 8.08 (s, 1H), 7.21 (t, 1H), 7.02 (d, 1H), 6.68 (d, 1H), 2.74 (s, 3H), 1.88 (s, 3H).

### Example 45. (P)-7-Amino-8-(3-hydroxy-2-methylphenyl)-3-methyl-[1,2,4]triazolo[4,3-a]pyridine-6-carboxamide (52) and (M)-7-amino-8-(3-hydroxy-2-methylphenyl)-3-methyl-[1,2,4]triazolo[4,3-a]pyridine-6-carboxamide (53)

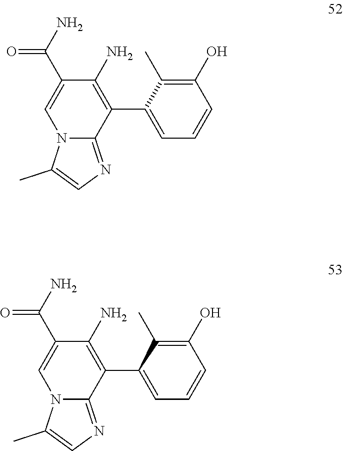

These two compounds were obtained by resolving compound 51 (racemate) using SFC separation (column: DAICEL CHIRALPAK IC (250 mm×30 mm, 10 um); mobile phase: [0.1% NH₃H₂O EtOH]; B %: 35%-35%).

Peak 1: 20.6 mg, SFC retention time=1.551 min. LC-MS m/z 298 (M+H⁺). ¹H NMR: (400 MHz, DMSO-d₆) δ=9.38 (s, 1H), 8.58 (m, 1H), 8.20 (s, 1H), 7.72 (s, 1H), 7.09 (t, 1H), 6.86 (d, 1H), 6.63 (d, 1H), 5.79 (s, 2H), 2.61 (s, 3H), 1.84 (s, 3H).

Peak 2: 27.3 mg, SFC retention time=2.700 min. LC-MS m/z 298 (M+H⁺). ¹H NMR: (400 MHz, DMSO-d₆) δ=9.56 (s, 1H), 8.85 (s, 1H), 8.42 (s, 1H), 7.89 (s, 1H), 7.37 (s, 1H), 7.15 (t, 1H), 7.13-7.00 (m, 1H), 6.94 (d, 1H), 6.67-6.65 (m, 1H), 2.66 (s, 3H), 1.86 (s, 3H).

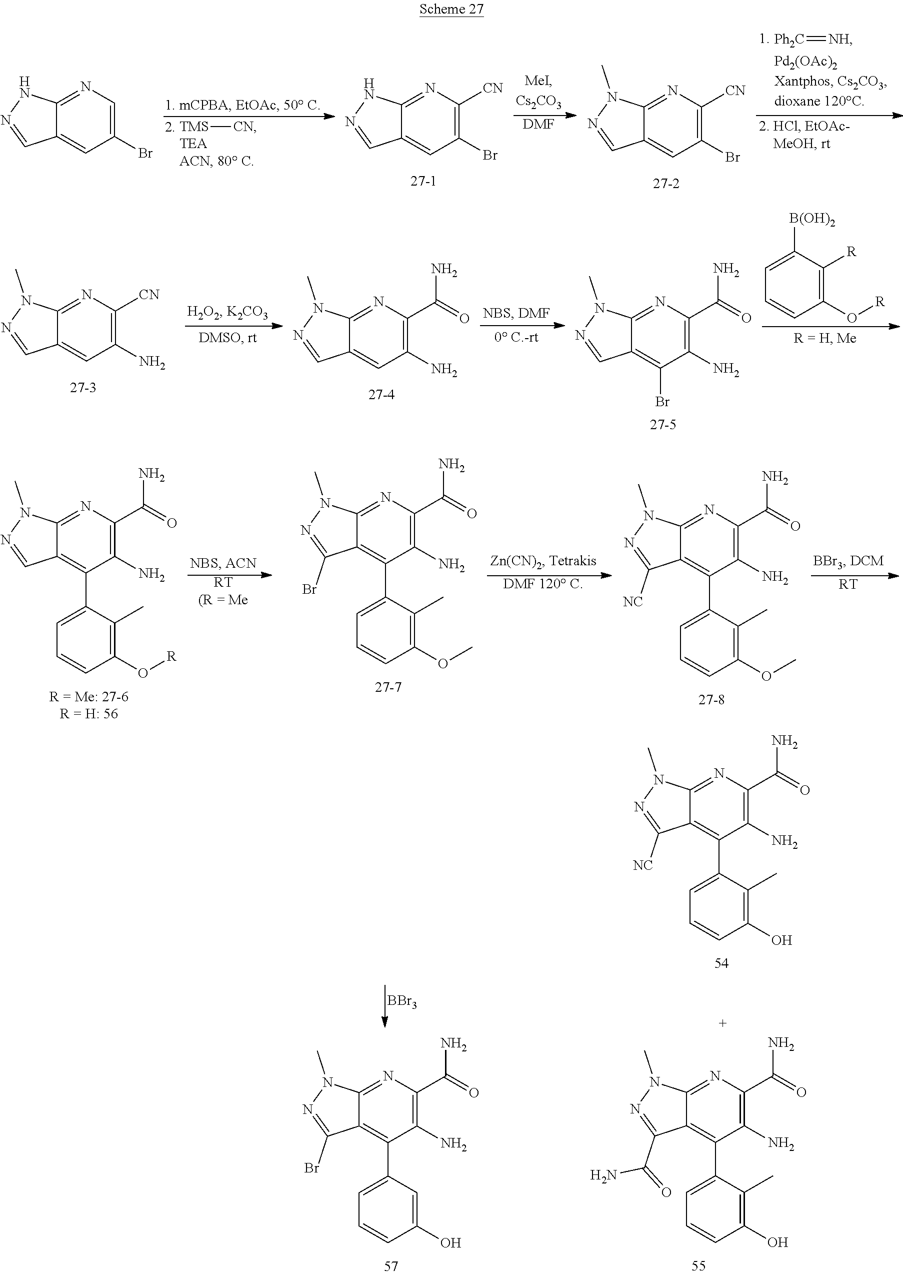

### Example 46. 5-Amino-3-cyano-4-(3-hydroxy-2-methylphenyl)-1-methyl-1H-pyrazolo[3,4-b]pyridine-6-carboxamide (54) and 5-amino-4-(3-hydroxy-2-methylphenyl)-1-methyl-1H-pyrazolo[3,4-b]pyridine-3,6-dicarboxamide (55)

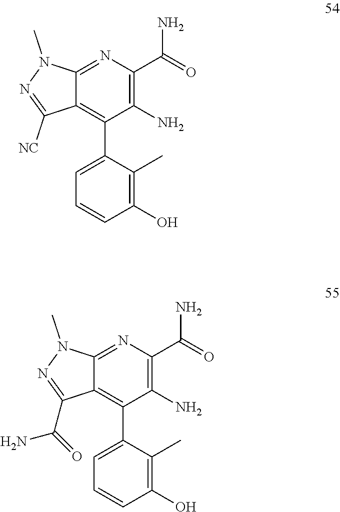 [SMILES: Cc1c(O)cccc1-c1c(N)c(C(N)=O)nc2c1c(C#N)nn2C ; Cc1c(O)cccc1-c1c(N)c(C(N)=O)nc2c1c(C(N)=O)nn2C]

### Step 1: Synthesis of 5-bromo-1H-pyrazolo[3,4-b]pyridine-6-carbonitrile

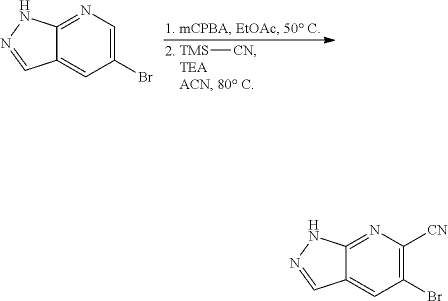 [SMILES: Brc1cnc2[nH]ncc2c1 ; C[Si](C)(C)C#N ; N#Cc1nc2[nH]ncc2cc1Br]

To a suspension of 5-bromo-1H-pyrazolo[3,4-b]pyridine (9.5 g, 48.0 mmol) in EtOAc (200 mL) was added m-CPBA (16.6 g, <75%) at room temperature over 5 min, and the reaction mixture was stirred at 45-50° C. for 15 h and cooled to room temperature. The resulting suspension was filtered. The filtered material was washed with EtOAc (2×10 mL) and dried under vacuum to afford 5-bromo-1H-pyrazolo[3,4-b]pyridine 7-oxide (9.10 g, 88.6% yield). LC-MS m/z 214/216 (M+H⁺).

To a suspension of 5-bromo-1H-pyrazolo[3,4-b]pyridine 7-oxide (9.10 g, 42.5 mmol) obtained above in MeCN (200 mL) was added TEA (8.60 g, 85.0 mmol) and TMSCN (10.6 ml, 85.0 mmol). The reaction mixture was stirred at 80° C. for 16 h. The cooled reaction mixture was concentrated in vacuo and the residue was suspended in MTBE and stirred for 8 h. The suspension was filtered, and the filtered material washed with MTBE (2×30 mL) and dried under vacuum to afford the title compound (27-1; 6.66 g, 70.2% yield). LC-MS m/z 223/225 (M+H⁺).

### Step 2: Synthesis of 5-bromo-1-methyl-1H-pyrazolo[3,4-b]pyridine-6-carbonitrile

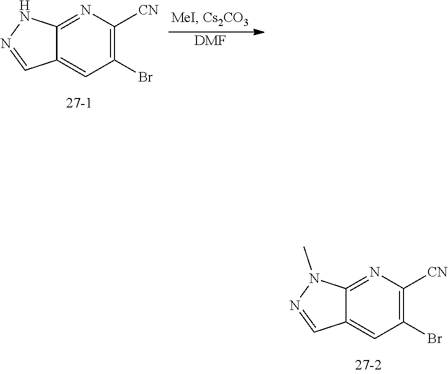

To a mixture of 5-bromo-1H-pyrazolo[3,4-b]pyridine-6-carbonitrile (2.27 g, 10.2 mmol), Cs₂CO₃ (8.15 g, 25 mmol) in anhydrous DMSO (40 mL) was added MeI (2.22 g, 15 mmol). The mixture was stirred at room temperature for 2 h. The reaction was quenched with water and the resulting suspension was filtered. The collected solid was washed with water and dried to give crude product, as a mixture of two regioisomers. The solid was extracted with DCM. The less polar isomer was in DCM extract, and the more polar one mainly remained as the solid. The DCM extract was evaporated, and the residue was subjected to silica gel column chromatography, eluted with DCM to give the pure title compound, 5-bromo-1-methyl-1H-pyrazolo[3,4-b]pyridine-6-carbonitrile (27-2.916 mg, 38% yield). ¹H NMR (400 MHz, DMSO) δ 8.89 (d, 1H), 8.34 (d, 1H), 4.10 (s, 3H). LCMS m/z 237/239 (M+H⁺).

### Step 3: Synthesis of 5-amino-1-methyl-1H-pyrazolo[3,4-b]pyridine-6-carbonitrile

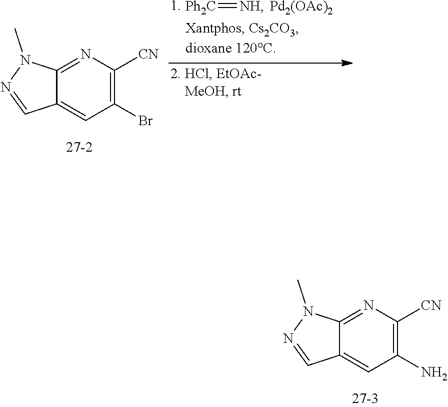

A mixture of 5-bromo-1-methyl-1H-pyrazolo[3,4-b]pyridine-6-carbonitrile (27-2, 807 mg, 3.40 mmol), benzophenone imine (1.54 g, 8.50 mmol), Pd₂(dba)₃ (0.311 mg, 0.34 mmol), XantPhos (0.393 g, 0.68 mmol). Cs₂CO₃ (3.31 g, 10.2 mmol) in dioxane (15 mL) was stirred at 120° C. under nitrogen until complete consumption of the starting material. The mixture was filtered through a Celite® pad and washed with EtOAc (2×30 mL). To the filtrate was added dropwise conc. HCl (3 mL) and the resulting mixture was stirred at room temperature overnight. The resulting suspension was filtered to remove the solid. The filtrate was basified with Na₂CO₃ (2M) to pH 9 and extracted with EtOAc (5×). The combined extracts were concentrated to dryness and the residue was subjected to silica gel column chromatography (10-90% EtOAc in hexane) to give 5-amino-1-methyl-1H-pyrazolo[3,4-b]pyridine-6-carbonitrile (27-3, 460 mg, 78% yield). ¹H NMR (400 MHz, DMSO) δ 8.03 (s, 1H), 7.61 (s, 1H), 5.77 (s, 2H), 3.97 (s, 3H). LC-MS m/z 174 (M+H⁺).

### Step 4: Synthesis of 5-amino-1-methyl-1H-pyrazolo[3,4-b]pyridine-6-carboxamide

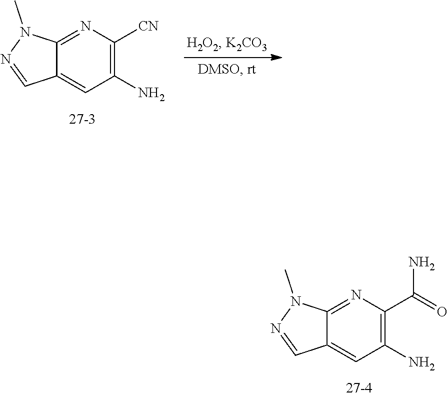

To a solution of 5-amino-1-methyl-pyrazolo[3,4-b]pyridine-6-carbonitrile (27-3, 0.430 g, 2.48 mmol) in DMSO (3 mL) was added potassium carbonate (0.90 g, 6.5 mmol) followed by adding hydrogen peroxide (0.90 g, 7.9 mmol, 30 mass %) dropwise, and resulting mixture was stirred until complete consumption of the starting material. The reaction was quenched with water and extracted with EtOAc (5×). The extract was dried with Na₂SO₄ and concentrated to give crude 5-amino-1-methyl-pyrazolo[3,4-b]pyridine-6-carboxamide (27-4, 0.380 g, 80.0% yield). ¹H NMR (400 MHz, DMSO) δ 8.34 (s, 1H), 7.92 (s, 1H), 7.60 (s, 1H), 7.44 (s, 1H), 6.47 (s, 2H), 4.02 (s, 3H). LC-MS m/z 192 (M+H⁺).

### Step 5: Synthesis of 5-amino-4-bromo-1-methyl-1H-pyrazolo[3,4-b]pyridine-6-carboxamide

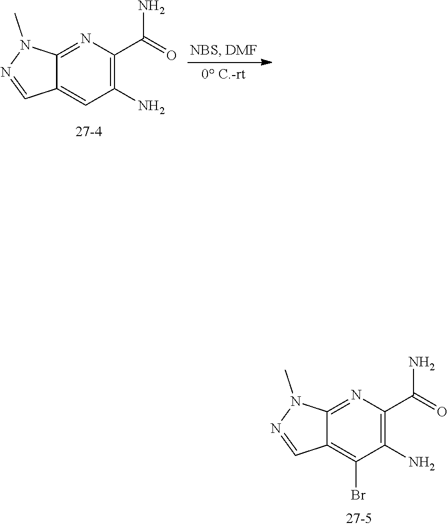

At 0° C., NBS (0.338 g, 1.90 mmol) was added to a solution of 5-amino-1-methyl-pyrazolo[3,4-b]pyridine-6-carboxamide (27-4, 0.36 g, 1.9 mmol) in ACN (8 mL) and the mixture was stirred at RT for 30 min. The reaction was quenched with water (40 mL) and the resulting suspension was filtered, the solid was washed with water and dried to give 5-amino-4-bromo-1-methyl-pyrazolo[3,4-b]pyridine-6-carboxamide (27-5, 0.420 g, 62.6% yield). LC-MS m/z 270/272 (M+H⁺).

### Step 6: Synthesis of 5-amino-4-(3-methoxy-2-methylphenyl)-1-methyl-1H-pyrazolo[3,4-b]pyridine-6-carboxamide

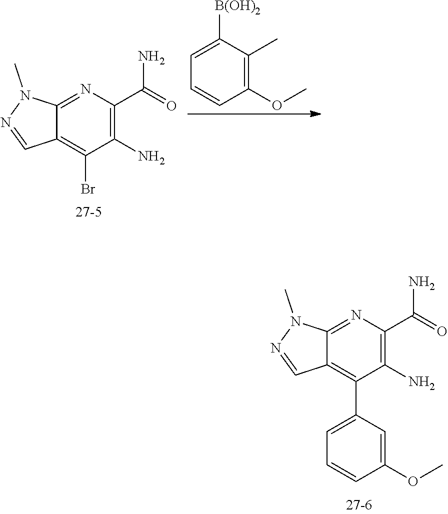

A mixture of 5-amino-4-bromo-1-methyl-pyrazolo[3,4-b]pyridine-6-carboxamide (27-5, 0.11 g, 0.41 mmol), (3-methoxy-2-methylphenyl)boronic acid (0.12 g, 0.72 mmol), dicyclohexyl-[2-(2,6-dimethoxyphenyl)phenyl]phosphine (20.0 mg, 0.0487 mmol), [2-(2-aminophenyl)phenyl]-methylsulfonyloxy-palladium; dicyclohexyl-[2-(2,6-dimethoxyphenyl)phenyl]phosphane (40.0 mg, 0.0513 mmol), and potassium phosphate (0.290 g, 1.37 mmol) in dioxane/water (3 mL, 5/1) was stirred under nitrogen at 115° C. until no starting material remaining (~1 h). The mixture was partitioned between EtOAc and water, the aqueous was extracted with EtOAc (3×). The combined organic extract was concentrated, and the residue purified by silica gel column chromatography (0-50% EtOAc in hexane) to give 5-amino-4-(3-methoxy-2-methylphenyl)-1-methyl-pyrazolo[3,4-b]pyridine-6-carboxamide (27-6, 87.0 mg, 0.279 mmol, 69% yield). ¹H NMR (400 MHz, DMSO) δ 8.48 (d, 1H), 7.72 (d, 1H), 7.42 (s, 1H), 7.36 (t, 1H), 7.10 (dd, 1H), 6.82 (dd, 1H), 5.91 (s, 2H), 4.07 (s, 3H), 3.87 (s, 3H), 1.88 (s, 3H); LC-MS m/z 312 (M+H⁺).

### Step 7: Synthesis of 5-amino-3-bromo-4-(3-methoxy-2-methylphenyl)-1-methyl-1H-pyrazolo[3,4-b]pyridine-6-carboxamide

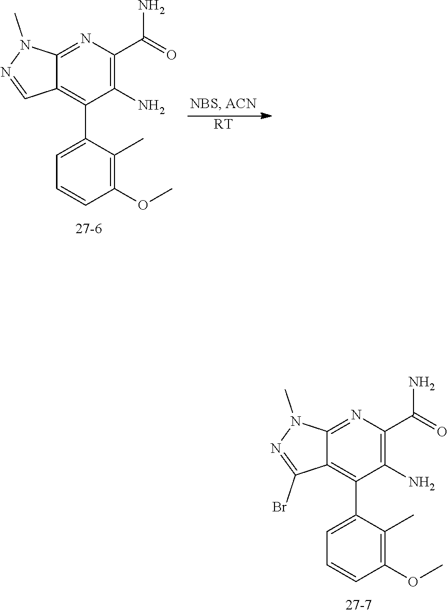 [SMILES: COc1cccc(-c2c(N)c(C(N)=O)nc3c2cnn3C)c1C ; "RT" ; COc1cccc(-c2c(N)c(C(N)=O)nc3c2c(Br)nn3C)c1C]

To a solution of 5-amino-4-(3-methoxy-2-methylphenyl)-1-methyl-pyrazolo[3,4-b]pyridine-6-carboxamide (27-6, 155 mg, 0.498 mmol) in ACN (10 mL) was added NBS solid (88 mg, 0.49 mmol) at RT; the reaction was stirred at RT for 20 min and concentrated to dryness. The residue was purified by silica gel column chromatography (0-40% EtOAc in hexane) to give 5-amino-3-bromo-4-(3-methoxy-2-methylphenyl)-1-methyl-1H-pyrazolo[3,4-b]pyridine-6-carboxamide (27-7, 126 mg, 65% yield). ¹H NMR (400 MHz, DMSO) δ 8.54 (s, 1H), 7.80 (s, 1H), 7.34 (t, 1H), 7.09 (d, 1H), 6.75 (dd, 1H), 5.90 (s, 2H), 4.06 (s, 3H), 3.86 (s, 3H), 1.82 (s, 3H). LC-MS m/z 390/392 (M+H⁺).

### Step 8: Synthesis of 5-amino-3-cyano-4-(3-methoxy-2-methylphenyl)-1-methyl-1H-pyrazolo[3,4-b]pyridine-6-carboxamide

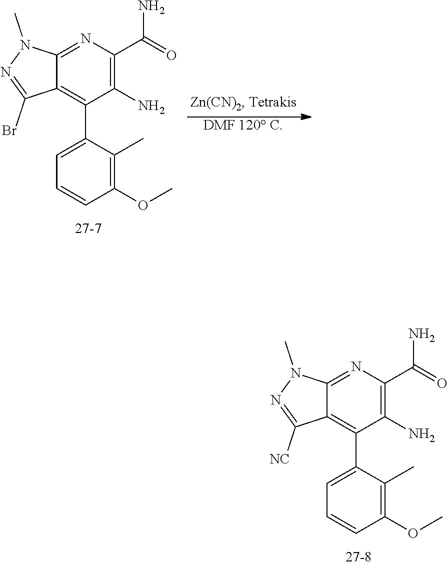 [SMILES: COc1cccc(-c2c(N)c(C(N)=O)nc3c2c(Br)nn3C)c1C ; "DMF 120° C." ; COc1cccc(-c2c(N)c(C(N)=O)nc3c2c(C#N)nn3C)c1C]

A mixture of 5-amino-3-bromo-4-(3-methoxy-2-methylphenyl)-1-methyl-pyrazolo[3,4-b]pyridine-6-carboxamide (27-7, 0.123 g, 0.315 mmol). Zn(CN)₂ (74 mg, 0.63 mmol). Pd(PPh₃)₄ (37 mg, 0.032 mmol) in DMF (3 mL) was stirred at 140° C. for 1 h and cooled to RT. EtOAc (15 mL) was added to the reaction and the resulting mixture was filtered through Celite®. The filtrate was concentrated, and the residue was purified by silica gel column chromatography (0-40% EtOAc in hexane) to give 5-amino-3-cyano-4-(3-methoxy-2-methylphenyl)-1-methyl-pyrazolo[3,4-b]pyridine-6-carboxamide (27-8, 92.0 mg, 0.274 mmol, 86.8% yield). ¹H NMR (400 MHz, DMSO) δ 8.63 (s, 1H), 7.88 (s, 1H), 7.36 (t, 1H), 7.12 (d, 1H), 6.82 (d, 1H), 6.27 (s, 2H), 4.21 (s, 3H), 3.87 (s, 3H), 1.87 (s, 3H). LCMS m/z 337 (M+H⁺).

### Step 9: Synthesis of 5-amino-3-cyano-4-(3-hydroxy-2-methylphenyl)-1-methyl-1H-pyrazolo[3,4-b]pyridine-6-carboxamide and 5-amino-4-(3-hydroxy-2-methylphenyl)-1-methyl-1H-pyrazolo[3,4-b]pyridine-3,6-dicarboxamide (Examples 54 and 55)

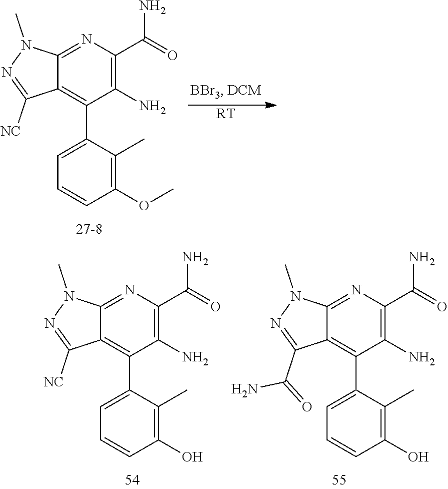 [SMILES: COc1cccc(-c2c(N)c(C(N)=O)nc3c2c(C#N)nn3C)c1C ; "RT" ; Cc1c(O)cccc1-c1c(N)c(C(N)=O)nc2c1c(C#N)nn2C ; Cc1c(O)cccc1-c1c(N)c(C(N)=O)nc2c1c(C(N)=O)nn2C]

To a solution of 5-amino-3-cyano-4-(3-methoxy-2-methylphenyl)-1-methyl-pyrazolo[3,4-b]pyridine-6-carboxamide (27-8, 92.0 mg, 0.274 mmol) in DCM (6 mL) was added BBr₃ (1M in DCM, 2.0 mL). The mixture was stirred at RT for 30 min, and quenched with NaHCO₃, extracted with EtOAc (3×). The combined organic extract was evaporated and the residue was purified by RP-HPLC (10-50% ACN in water, 0.1% FA) to give 5-amino-3-cyano-4-(3-hydroxy-2-methylphenyl)-1-methyl-pyrazolo[3,4-b]pyridine-6-carboxamide (compound 54, 52.0 mg, 59.0% yield), LCMS m/z 323 (M+H⁺), ¹H NMR (400 MHz, DMSO) δ 9.61 (s, 1H), 8.62 (d, 1H), 7.88 (d, 1H), 7.17 (t, 1H), 6.95 (dd, 1H), 6.66 (dd, 1H), 6.23 (s, 2H), 4.21 (s, 3H), 1.83 (s, 3H), and 5-amino-4-(3-hydroxy-2-methylphenyl)-1-methyl-pyrazolo[3,4-b]pyridine-3,6-dicarboxamide (compound 55, 6.0 mg, 6.4% yield), LCMS m/z 341 (M+H⁺), ¹H NMR (400 MHz, DMSO) δ 9.29 (s, 1H), 8.46 (d, 1H), 7.69 (d, 1H), 7.13 (s, 1H), 6.96 (t, 1H), 6.85 (s, 1H), 6.75 (dd, 1H), 6.39 (dd, 1H), 5.73 (s, 2H), 4.05 (s, 3H), 1.69 (s, 3H).

### Example 47. 5-Amino-4-(3-hydroxy-2-methylphenyl)-1-methyl-pyrazolo[3,4-b]pyridine-6-carboxamide (56)

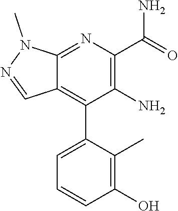 [SMILES: Cc1c(O)cccc1-c1c(N)c(C(N)=O)nc2c1cnn2C]

The title compound was synthesized by the same method depicted in Scheme 27, using (3-hydroxy-2-methylphenyl)boronic acid replacing (3-methoxy-2-methylphenyl)boronic acid in step 6.

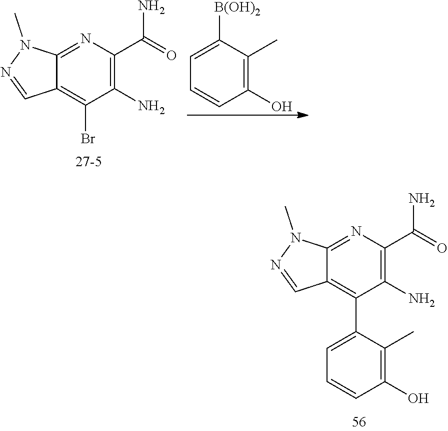

A solution of 5-amino-4-bromo-1-methyl-pyrazolo[3,4-b]pyridine-6-carboxamide (27-5, 0.106 g, 0.392 mmol), (3-hydroxy-2-methylphenyl)boronic acid (0.120 g, 0.790 mmol), dicyclohexyl-[2-(2,6-dimethoxyphenyl)phenyl]phosphine (20.0 mg, 0.0487 mmol), [2-(2-aminophenyl)phenyl]-methylsulfonyloxy-palladium; dicyclohexyl-[2-(2,6-dimethoxyphenyl)phenyl]phosphane (35.0 mg, 0.0449 mmol), and potassium phosphate (0.330 g, 1.55 mmol) in 5:1 dioxane/water (3 mL) was stirred at 115° C. until no starting material remained (~1 h). The mixture was partitioned between EtOAc and water. The aqueous was extracted with EtOAc (3×). The organic extract was concentrated, and the residue was purified by silica gel column chromatography (0-100% EtOAc in hexane) to give 5-amino-4-(3-hydroxy-2-methylphenyl)-1-methyl-pyrazolo[3,4-b]pyridine-6-carboxamide (95.0 mg, 81.4% yield). ¹H NMR (400 MHz, DMSO) δ 9.62 (s, 1H), 8.47 (d, 1H), 7.71 (d, 1H), 7.43 (s, 1H), 7.17 (t, 1H), 6.93 (dd, 1H), 6.66 (dd, 1H), 5.87 (s, 2H), 4.07 (s, 3H), 1.84 (s, 3H). LCMS m/z 298 (M+H⁺).

### Example 48. 5-Amino-3-bromo-4-(3-hydroxy-2-methylphenyl)-1-methyl-1H-pyrazolo[3,4-b]pyridine-6-carboxamide (57)

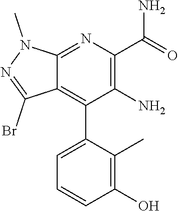 [SMILES: Cc1c(O)cccc1-c1c(N)c(C(N)=O)nc2c1c(Br)nn2C]

Compound 57 was synthesized using compound 27-7 as depicted in Scheme 27. To a mixture of 5-amino-3-bromo-4-(3-methoxy-2-methylphenyl)-1-methyl-pyrazolo[3,4-b]pyridine-6-carboxamide (27-7, 15.0 mg, 0.0384 mmol) dissolved in DCM (6 mL) was added BBr₃ (1M in DCM, 0.4 mL). The mixture was stirred until no starting material remained (~30 min). The reaction was quenched with NaHCO₃ and extracted with EtOAc (3×). The combined organic extract was evaporated, and the residue was purified by RP-HPLC (10-50% ACN in water, 01% FA) to give 5-amino-3-bromo-4-(3-hydroxy-2-methylphenyl)-1-methyl-pyrazolo[3,4-b]pyridine-6-carboxamide (8.7 mg, 60% yield). ¹H NMR (400 MHz, DMSO) δ 9.48 (s, 1H), 8.46 (d, 1H), 7.72 (s, 1H), 7.08 (t, 1H), 6.85 (dd, 1H), 6.51 (dd, 1H), 5.79 (s, 2H), 3.99 (s, 3H), 1.70 (s, 3H). LCMS m/z 376/378 (M+H⁺).

### Example 49. (M)-5-Amino-3-cyano-4-(3-hydroxy-2-methylphenyl)-1-methyl-1H-pyrazolo[3,4-b]pyridine-6-carboxamide (58) and (P)-5-amino-3-cyano-4-(3-hydroxy-2-methylphenyl)-1-methyl-1H-pyrazolo[3,4-b]pyridine-6-carboxamide (59)

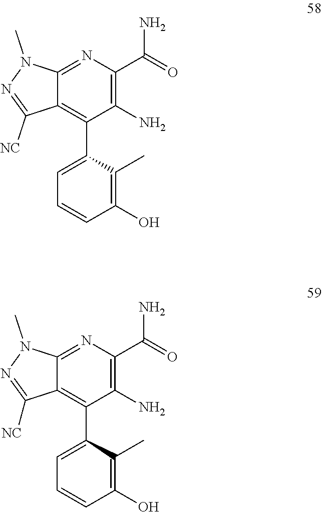

Compounds 58 and 59 were obtained by chiral resolution of compound 54 (50 mg, a racemate). Chiral SFC (Instrument: Waters SFC Prep 150 Mgm; Column: Chiralcel OD, 30×150 mm, 5 um; Conditions: Isocratic at 40% methanol with 60% CO₂; Flow Rate: 100 mL/min) provided peak 1 (22 mg. Rt: 1.92 min) and peak 2 (21 mg, Rt: 2.34 min).

Peak 1: 22 mg, ¹H NMR (400 MHz, DMSO) δ 9.55 (s, 1H), 8.55 (d, 1H), 7.81 (d, 1H), 7.10 (t, 1H), 6.88 (dd, 1H), 6.58 (dd, 1H), 6.16 (s, 2H), 4.13 (s, 3H), 1.76 (s, 3H). LCMS m/z 323 (M+H⁺).

Peak 2: 21 mg. ¹H NMR (400 MHz, DMSO) δ 9.55 (s, 1H), 8.56 (d, 1H), 7.81 (d, 1H), 7.10 (t, 1H), 6.88 (dd, 1H), 6.58 (dd, 1H), 6.16 (s, 2H), 4.13 (s, 3H), 1.76 (s, 3H). LCMS m/z 323 (M+H⁺).

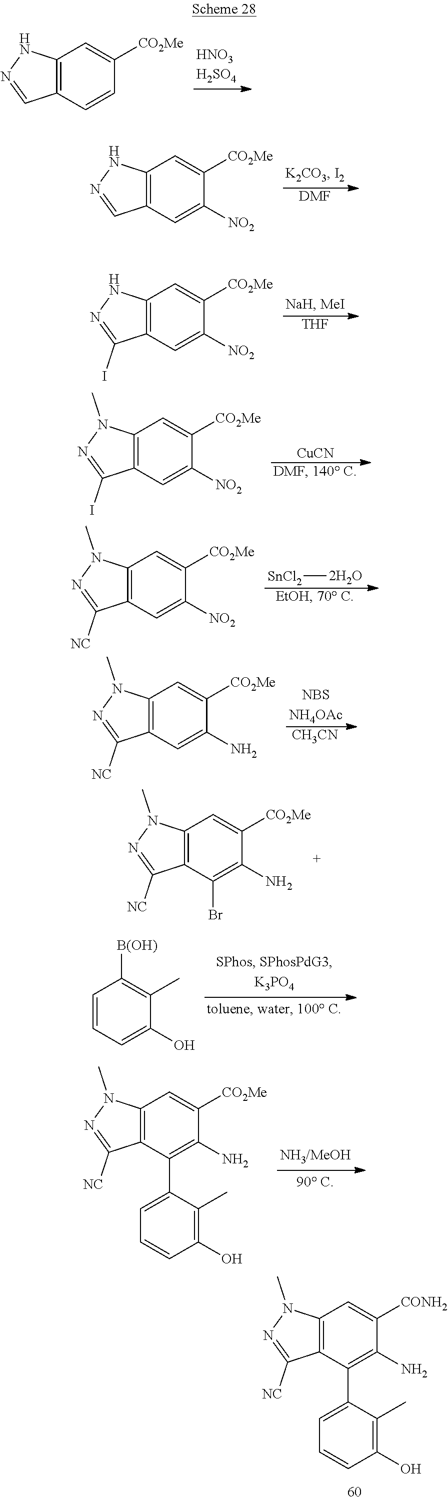

### Example 50. Methyl 5-amino-3-cyano-4-(3-hydroxy-2-methylphenyl)-1-methyl-1H-indazole-6-carboxylate (60)

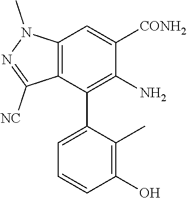 [SMILES: Cc1c(O)cccc1-c1c(N)c(C(N)=O)cc2c1c(C#N)nn2C]

### Step 1: Synthesis of methyl 5-nitro-1H-indazole-6-carboxylate

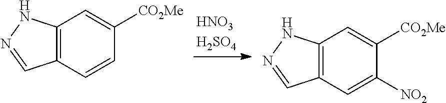 [SMILES: COC(=O)c1ccc2cn[nH]c2c1 ; COC(=O)c1cc2[nH]ncc2cc1[N+](=O)[O-]]

To a mixture of methyl 1H-indazole-6-carboxylate (10.2 g, 57.9 mmol, 1.00 equiv.) and concentrated H₂SO₄ (100 mL) was added a solution of HNO₃ (10 mL, 69% purity) and H₂SO₄ (20 mL) over 30 min at −10° C. to 0° C. The resulting mixture was stirred at the same temperature for an additional 1 h, and then slowly poured into ice water (1000 mL). The suspension was filtered, and the filter cake was washed with water (2×200 mL). The solid was collected and dried to give the title compound (13.0 g, 102% yield). LC-MS m/z 222 (M+H⁺).

### Step 2: Synthesis of methyl 3-iodo-5-nitro-1H-indazole-6-carboxylate

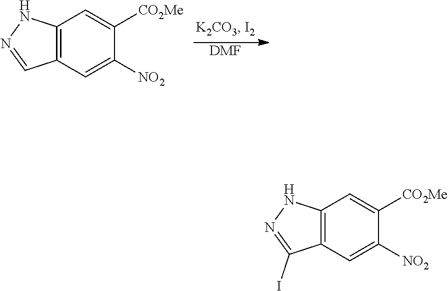

A mixture of methyl 5-nitro-1H-indazole-6-carboxylate (4 g, 18.1 mmol, 1.00 equiv.) and K₂CO₃ (7.5 g, 54.3 mmol, 3.00 equiv.) in anhydrous DMF (50 mL) was cooled in ice bath. Iodine (13.7 g, 54.3 mmol, 3.00 equiv.) was added in portions, and the resulting mixture was stirred overnight. The reaction was poured into 400 mL of 5% NaHSO₃, and the resulting light-yellow precipitate was isolated by filtration, washed with water, and dried under high vacuum to give the title compound (5.33 g, 81% yield). LC-MS m/z 348 (M+H⁺).

### Step 3: Synthesis of methyl 3-iodo-1-methyl-5-nitro-1H-indazole-6-carboxylate

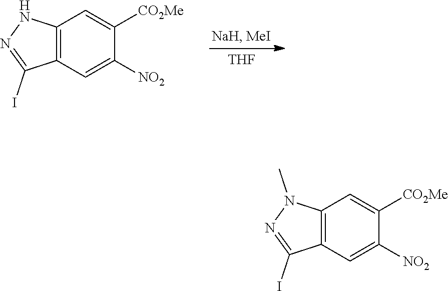 [SMILES: COC(=O)c1cc2[nH]nc(I)c2cc1[N+](=O)[O-] ; COC(=O)c1cc2c(cc1[N+](=O)[O-])c(I)nn2C ; "THF"]

A mixture of methyl 3-iodo-5-nitro-1H-indazole-6-carboxylate (3 g, 8.64 mmol, 1.00 equiv.) and anhydrous THF (28 mL) was placed in ice bath. NaH (415 mg, 10.4 mmol, 1.20 equiv., 60% dispersion in oil) was slowly added. After gas evolution had stopped, iodomethane (0.75 mL, 12.0 mmol, 1.40 equiv.) was added, and the mixture was stirred for 3 days at room temperature. The reaction was quenched with saturated ammonium chloride and extracted with EtOAc (3×). The organic layers were combined, dried over MgSO₄, filtered and concentrated. The residue was adsorbed onto silica and subjected to FCC (DCM:hexane 15:85 to 100:0) to give the title compound (1.20 g, 38% yield). LC-MS m/z 362 (M+H⁺).

### Step 4: Synthesis of methyl 3-cyano-1-methyl-5-nitro-1H-indazole-6-carboxylate

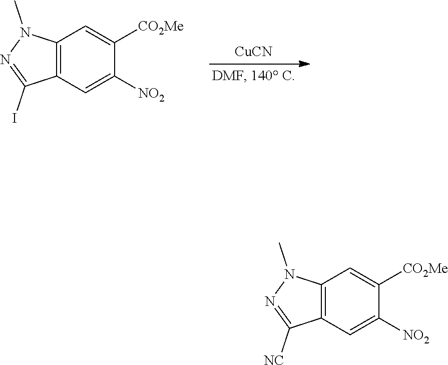 [SMILES: COC(=O)c1cc2c(cc1[N+](=O)[O-])c(I)nn2C ; "CuCN" ; "DMF, 140° C." ; COC(=O)c1cc2c(cc1[N+](=O)[O-])c(C#N)nn2C]

A round bottom flask was charged with methyl 3-iodo-1-methyl-5-nitro-1H-indazole-6-carboxylate (1203 mg, 3.33 mmol, 1.00 equiv.) and CuCN (750 mg, 8.37 mmol, 2.51 equiv.). The flask was evacuated and refilled with nitrogen. Anhydrous DMF (11 mL) was then added, and the mixture was heated at 140° C. for 2 h under nitrogen. The reaction was cooled to room temperature and poured into a vigorously stirring mixture of 1M HCl (200 mL) and EtOAc (150 mL). Stirring was continued until most solid had dissolved. The aqueous layer was extracted with additional EtOAc (150 mL), and the organic layers were combined, dried over MgSO₄, filtered and concentrated. The residue was adsorbed onto silica and subjected to FCC (EtOAc:hexane 5:95 to 60:40) to give the title compound (555 mg, 64% yield). LC-MS m/z 261 (M+H⁺).

### Step 5: Synthesis of methyl 5-amino-3-cyano-1-methyl-1H-indazole-6-carboxylate

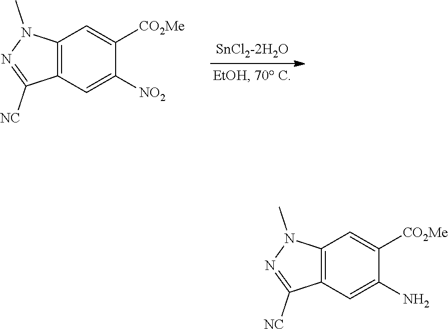 [SMILES: COC(=O)c1cc2c(cc1[N+](=O)[O-])c(C#N)nn2C ; "EtOH, 70° C." ; "SnCl2-2H2O" ; COC(=O)c1cc2c(cc1N)c(C#N)nn2C]

A mixture of methyl 3-cyano-1-methyl-5-nitro-1H-indazole-6-carboxylate (500 mg, 1.92 mmol, 1.00 equiv.) and tin(II) chloride dihydrate (2170 mg, 9.62 mmol, 5.00 equiv.) in absolute EtOH (50 mL) was stirred at room temperature for 30 min, then at 70° C. for 25 min. The reaction was cooled to room temperature, concentrated to approximately half its original volume, and then partitioned between EtOAc and saturated NaHCO₃ to give a suspension. The solid was removed by filtration, and the layers were separated. The aqueous layer was extracted with additional EtOAc, and the organic layers were combined, dried over MgSO₄, filtered and concentrated. The residue was adsorbed onto silica and subjected to FCC (EtOAc:hexane 5:95 to 50:50) to give the title compound (306 mg, 69% yield). LC-MS m/z 231 (M+H⁺).

### Step 6: Synthesis of methyl 5-amino-4-bromo-3-cyano-1-methyl-1H-indazole-6-carboxylate

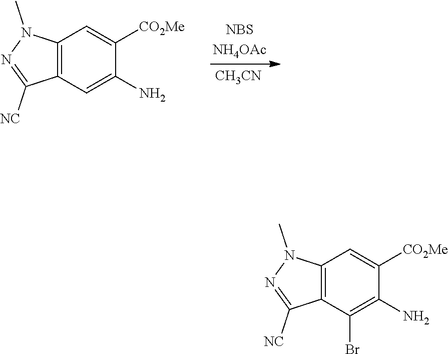

To a mixture of methyl 5-amino-3-cyano-1-methyl-1H-indazole-6-carboxylate (302 mg, 1.31 mmol, 1.00 equiv.), NBS (250 mg, 1.40 mmol, 1.07 equiv.) and ammonium acetate (11 mg, 0.14 mmol, 0.11 equiv.) was added CH₃CN (30 mL), and the mixture was stirred at room temperature for 20 min. The reaction was concentrated, and the residue was adsorbed onto silica and subjected to FCC (EtOAc:DCM 0:100 to 5:95) to give the title compound (293 mg, 72% yield). LC-MS m/z 309 (M+H⁺).

### Step 7: Synthesis of methyl 5-amino-3-cyano-4-(3-hydroxy-2-methylphenyl)-1-methyl-1H-indazole-6-carboxylate

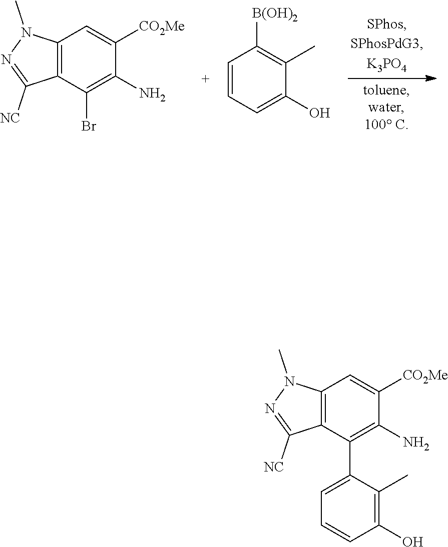 [SMILES: COC(=O)c1cc2c(c(C#N)nn2C)c(Br)c1N ; Cc1c(O)cccc1B(O)O ; COC(=O)c1cc2c(c(C#N)nn2C)c(-c2cccc(O)c2C)c1N]

A microwave vial was charged with methyl 5-amino-4-bromo-3-cyano-1-methyl-1H-indazole-6-carboxylate (100 mg, 0.32 mmol, 1.00 equiv.), (3-hydroxy-2-methylphenyl)boronic acid (100 mg, 0.66 mmol, 2.03 equiv.), dicyclohexyl(2′,6′-dimethoxy[1,1′-biphenyl]-2-yl)phosphane (SPhos, 27 mg, 0.066 mmol, 0.20 equiv.). (2-dicyclohexylphosphino-2′,6′-dimethoxybiphenyl) [2-(2′-amino-1,1′-biphenyl)]palladium(II) methanesulfonate (SPhos Pd G3, 51 mg, 0.065 mmol, 0.20 equiv.) and K₃PO₄ (140 mg, 0.66 mmol, 2.04 equiv.). The vial was evacuated and refilled with nitrogen. Degassed toluene (1.6 mL) and water (0.16 mL) were added, and the mixture was sealed and heated in the microwave to 100° C. for 1 hour. The resulting suspension was partitioned between EtOAc (10 mL) and water (10 mL). The aqueous layer was extracted with more EtOAc, and the organic layers were combined, dried over MgSO₄, filtered and concentrated. The residue was adsorbed onto silica and subjected to FCC (EtOAc:hexane 5:95 to 50:50) to give the title compound (92 mg, 85% yield). LC-MS m/z 337 (M+H⁺).

### Step 8: Synthesis of 5-amino-3-cyano-4-(3-hydroxy-2-methylphenyl)-1-methyl-1H-indazole-6-carboxamide (compound 60)

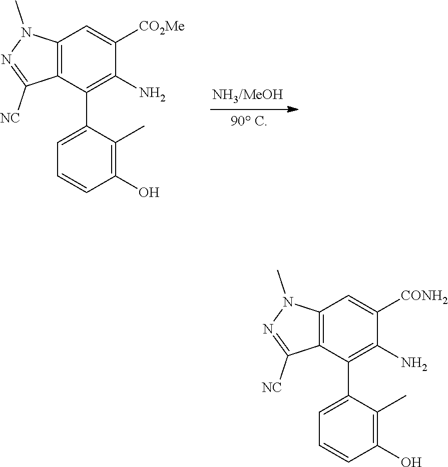 [SMILES: COC(=O)c1cc2c(c(C#N)nn2C)c(-c2cccc(O)c2C)c1N ; Cc1c(O)cccc1-c1c(N)c(C(N)=O)cc2c1c(C#N)nn2C]

Methyl 5-amino-3-cyano-4-(3-hydroxy-2-methylphenyl)-1-methyl-1H-indazole-6-carboxylate (92 mg, 0.27 mmol, 1.00 equiv.) was suspended in a solution of 7 M ammonia in MeOH (10 mL, 70 mmol, 256 equiv.) and heated at 50° C. (oil bath temperature) for 1 h. Additional 7 M ammonia solution (5 mL, 35 mmol, 128 equiv.) was added, and the mixture was heated in the microwave at 90° C. for 12 hours. The solvent was removed, and the residue was dissolved in MeOH, filtered, and subjected to RP-HPLC (0.1% FA, water:MeCN, 90:10 to 40:60) to afford the title compound (47 mg, 53% yield). ¹H NMR (400 MHz, DMSO) δ 9.46 (s, 1H), 8.16 (s, 1H), 8.09 (s, 1H), 7.67 (s, 1H), 7.12 (t. J=7.8 Hz, 1H), 6.89 (dd, J=8.2, 1.3 Hz, 1H), 6.59 (dd, J=7.6, 1.2 Hz, 1H), 5.43 (s, 2H), 4.13 (s, 3H), 1.79 (s, 3H). LCMS m/z 322 (M+H⁺).

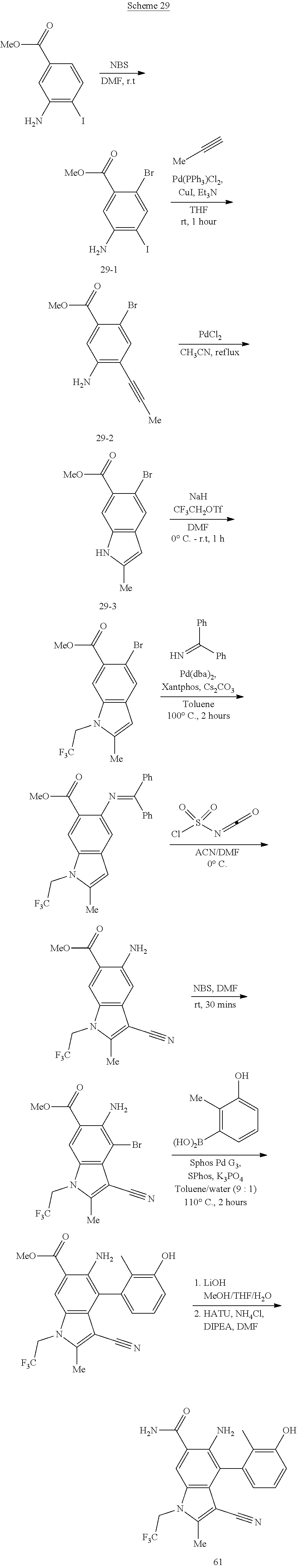

### Example 51. 5-Amino-3-cyano-4-(3-hydroxy-2-methylphenyl)-2-methyl-1-(2,2,2-trifluoroethyl)-1H-indole-6-carboxamide (61)

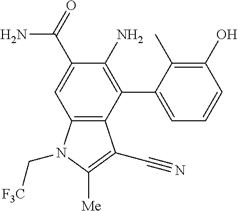 [SMILES: Cc1c(O)cccc1-c1c(N)c(C(N)=O)cc2c1c(C#N)c(C)n2CC(F)(F)F]

### Step 1: Synthesis of methyl 5-amino-2-bromo-4-iodo-benzoate

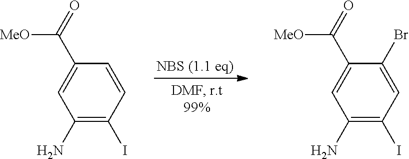 [SMILES: COC(=O)c1ccc(I)c(N)c1 ; COC(=O)c1cc(N)c(I)cc1Br]

To a solution of methyl 3-amino-4-iodo-benzoate (10 g, 36.1 mmol) in DMF (50 mL) was added 1-bromopyrrolidine-2,5-dione (NBS, 7.1 g, 1.1 equiv., 39.7 mmol). The mixture was stirred at room temperature for 2 hours. Upon completion, the mixture was poured into 500 mL water and a solid was obtained. The mixture was filtered then the filter cake was washed with water (3×50 mL) and dried in vacuo to give the title compound methyl 5-amino-2-bromo-4-iodo-benzoate (29-1, 12.8 g, 99% yield). LC-MS m/z [M+H]⁺=355.95.

### Step 2: Synthesis of methyl 5-amino-2-bromo-4-prop-1-ynyl-benzoate

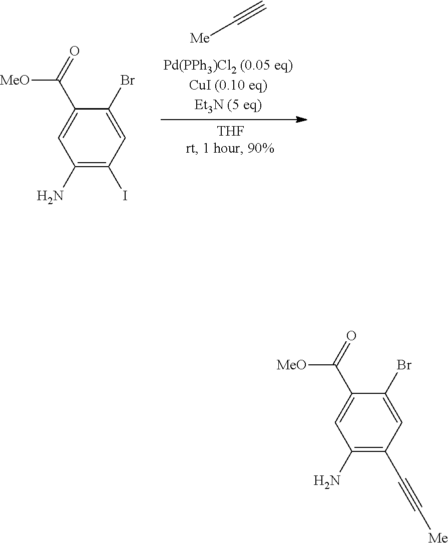

A solution of prop-1-yne (33 ml 1N in DMF, 3.0 equiv., 33.7 mmol), triethylamine (9.4 ml, 6.0 equiv., 67.4 mmol), and copper(I) iodide (214 mg, 0.1 equiv., 1.1 mmol) was added to a yellow solution of methyl 5-amino-2-bromo-4-iodo-benzoate (29-1, 4 g, 11.2 mmol) in THF (50 mL) at ambient temperature to form a yellow suspension. After stirring for 5 minutes, Pd(PPh₃)₂Cl₂ (394 mg, 0.05 equiv., 0.56 mmol) was added to the reaction mixture at ambient temperature to afford a black suspension. The reaction mixture was stirred at ambient temperature for 45 min. quenched with water and extracted with EtOAc (2×). The EtOAc extract was washed with brine, dried over MgSO₄ and concentrated. The residue was purified by column chromatography (0-20% EtOAc in hexane) to give the title compound methyl 5-amino-2-bromo-4-prop-1-ynyl-benzoate (29-2, 2.7 g, 90% yield). LC-MS m/z [M+H]⁺=270.

### Step 3: Synthesis of methyl 5-bromo-2-methyl-1H-indole-6-carboxylate

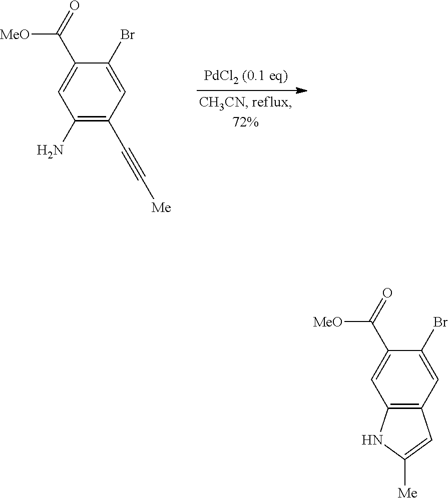 [SMILES: CC#Cc1cc(Br)c(C(=O)OC)cc1N ; COC(=O)c1cc2[nH]c(C)cc2cc1Br]

A solution of methyl 5-amino-2-bromo-4-prop-1-ynyl-benzoate (2.7 g, 10.1 mmol) and dichloropalladium (178 mg, 0.1 equiv., 1.01 mmol) in acetonitrile (50 mL) was heated at 85° C. under nitrogen atmosphere until complete disappearance of the starting material (NMR monitoring). The mixture was cooled to room temperature and the solvent was removed under reduced pressure. The residue was purified by column chromatography (0-20% EtOAc in hexane) to give the title compound methyl 5-bromo-2-methyl-1H-indole-6-carboxylate (29-3, 1.95 g, 72%). ¹H NMR (400 MHz, DMSO) δ 11.46 (s, 1H), 7.81 (d, J=2.8 Hz, 1H), 7.74 (d, J=2.7 Hz, 1H), 6.21 (s, 1H), 3.84 (d, J=2.7 Hz, 3H), 2.43 (d, J=2.8 Hz, 3H). LC-MS m/z [M+H]⁺=270.

### Step 4: Synthesis of methyl 5-bromo-2-methyl-1-(2,2,2-trifluoroethyl)indole-6-carboxylate

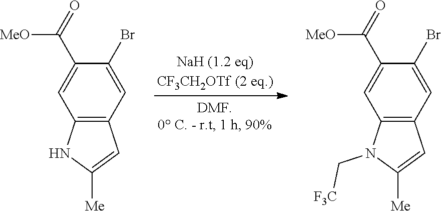 [SMILES: COC(=O)c1cc2[nH]c(C)cc2cc1Br ; COC(=O)c1cc2c(cc1Br)cc(C)n2CC(F)(F)F]

To a solution of methyl 5-bromo-2-methyl-1H-indole-6-carboxylate (29-3, 800 mg, 3.0 mmol) in DMF (5 ml) was added 60% sodium hydride (143 mg, 1.2 equiv., 3.6 mmol) at 0° C. The reaction was warmed slowly to room temperature and stirred for 30 minutes. The reaction was re-cooled to 0° C. followed by addition of 2,2,2-trifluoroethyl trifluoromethanesulfonate (1.39 g, 2.0 equiv., 6.0 mmol) dropwise. The mixture was stirred at room temperature until completion, quenched with water (50 mL) and extracted with ethyl acetate (3×). The combined organic extract was washed with water and brine, dried over anhydrous sodium sulfate, and concentrated under reduced pressure. The residue was purified by column chromatography (0-20% EtOAc in hexane) to give the title compound methyl 5-bromo-2-methyl-1-(2,2,2-trifluoroethyl)indole-6-carboxylate (900 mg, 90% yield). LC-MS m/z [M+H]⁺=350.10.

### Step 5: Synthesis of methyl 5-(benzhydrylideneamino)-2-methyl-1-(2,2,2-trifluoroethyl)indole-6-carboxylate

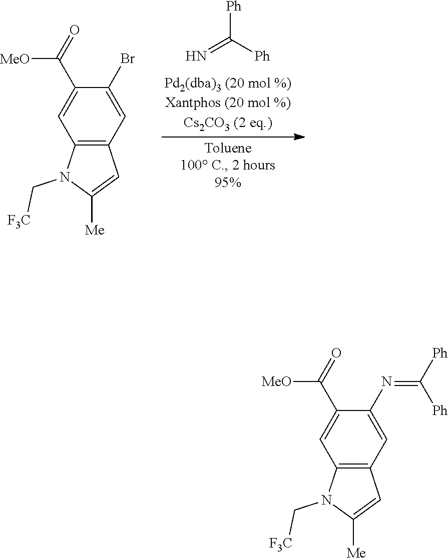 [SMILES: COC(=O)c1cc2c(cc1Br)cc(C)n2CC(F)(F)F ; N=C(c1ccccc1)c1ccccc1 ; COC(=O)c1cc2c(cc1N=C(c1ccccc1)c1ccccc1)cc(C)n2CC(F)(F)F]

To a solution of methyl 5-bromo-2-methyl-1-(2,2,2-trifluoroethyl)indole-6-carboxylate (0.9 g, 2.6 mmol) and diphenylmethanimine (0.9 ml, 2.0 equiv., 5.2 mmol) in toluene (5 mL) was added cesium carbonate (1.7 g, 2.0 equiv., 5.2 mmol), Pd₂(dba)₃ (470 mg, 0.2 equiv., 0.52 mmol). Xantphos (297 mg, 0.2 equiv., 0.52 mmol). The mixture was stirred at 100° C. for 2 hours, cooled to room temperature, quenched with water, and extracted with EtOAc (2×). The EtOAc extract was washed with brine and concentrated. The residue was purified by column chromatography (0-20% EtOAc in hexane) to give the title compound methyl 5-(benzhvdrylideneamino)-2-methyl-1-(2,2,2-trifluoroethyl)indole-6-carboxylate (1.1 g, 95% yield). LC-MS m/z [M+H]⁺=451.20.

### Step 6: Synthesis of methyl 5-amino-3-cyano-2-methyl-1-(2,2,2-trifluoroethyl)indole-6-carboxylate

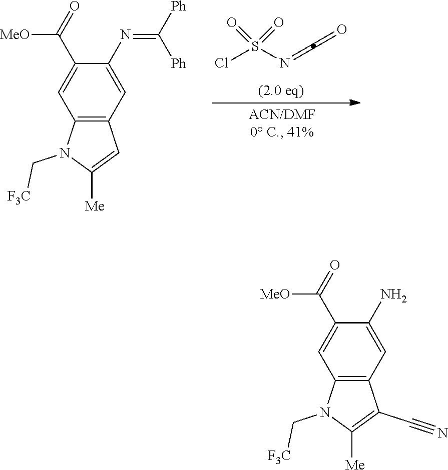

To a solution of methyl 5-(benzhydrylideneamino)-2-methyl-1-(2,2,2-trifluoroethyl)indole-6-carboxylate (1.1 g, 2.4 mmol) solubilized in anhydrous acetonitrile (10 mL) was added dropwise N-(oxomethylene)sulfamoyl chloride (0.45 ml, 2.0 equiv., 4.8 mmol) at 0° C. The resulting mixture was stirred at 0° C. for 2 hours followed by dropwise addition of DMF (5 mL). The mixture was stirred at 0° C. for another 1 hour, quenched with water and extracted with EtOAc (2×). The EtOAc extract was washed with brine and concentrated. The residue was purified by column chromatography (0-50% EtOAc in hexane) to give the title compound methyl 5-amino-3-cyano-2-methyl-1-(2,2,2-trifluoroethyl)indole-6-carboxylate (350 mg, 41% yield). LC-MS m/z [M+H]⁺=312.10.

### Step 7: Synthesis of methyl 5-amino-4-bromo-3-cyano-2-methyl-1-(2,2,2-trifluoroethyl)indole-6-carboxylate

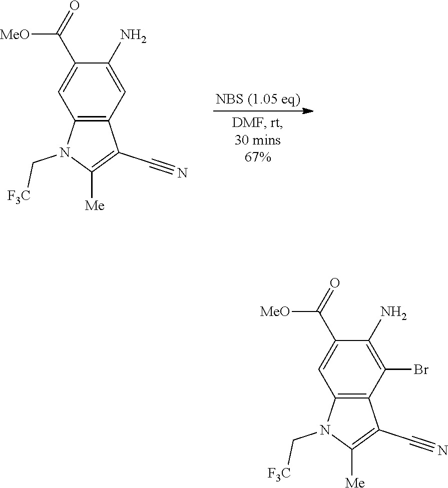 [SMILES: COC(=O)c1cc2c(cc1N)c(C#N)c(C)n2CC(F)(F)F ; COC(=O)c1cc2c(c(Br)c1N)c(C#N)c(C)n2CC(F)(F)F]

To a solution of methyl 5-amino-3-cyano-2-methyl-1-(2,2,2-trifluoroethyl)indole-6-carboxylate (250 mg, 0.8 mmol) in DMF (5 mL) was added 1-bromopyrrolidine-2,5-dione (NBS, 157 mg, 1.05 equiv., 0.85 mmol). The reaction was stirred for 30 min at room temperature, quenched with water and extracted with EtOAc (2×). The EtOAc extract was washed with brine and concentrated. The residue was purified by column chromatography (0-50% EtOAc in hexane) to get the title compound methyl 5-amino-4-bromo-3-cyano-2-methyl-1-(2,2,2-trifluoroethyl)indole-6-carboxylate (210 mg, 67% yield). LC-MS m/z [M+H]⁺=391.90.

### Step 8: Synthesis of methyl 5-amino-3-cyano-4-(3-hydroxy-2-methylphenyl)-2-methyl-1-(2,2,2-trifluoroethyl)indole-6-carboxylate

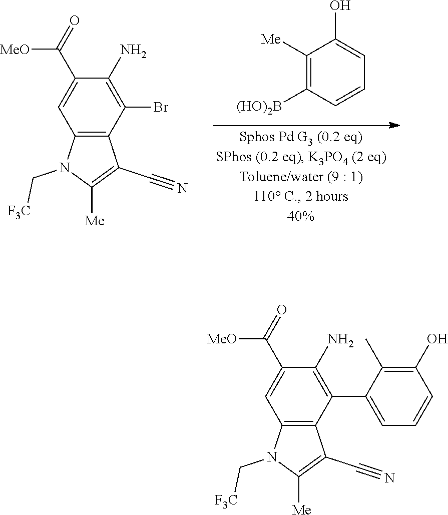 [SMILES: COC(=O)c1cc2c(c(Br)c1N)c(C#N)c(C)n2CC(F)(F)F ; Cc1c(O)cccc1B(O)O ; COC(=O)c1cc2c(c(C#N)c(C)n2CC(F)(F)F)c(-c2cccc(O)c2C)c1N]

To a solution of methyl 5-amino-4-bromo-3-cyano-2-methyl-1-(2,2,2-trifluoroethyl)indole-6-carboxylate (150 mg, 0.38 mmol), potassium phosphate (163 mg, 2.0 equiv., 0.76 mmol) and (3-hydroxy-2-methylphenyl)boronic acid (116 mg, 2.0 equiv., 0.76 mmol) in the toluene (2 mL) and water (0.2 mL) was added the SPhos Pd G₃ (60 mg, 0.2 equiv., 0.076 mmol), SPhos (32 mg, 0.2 equiv., 0.076 mmol) under nitrogen. The mixture was stirred at 110° C. for 2 hours, cooled to room temperature, quenched with water, and extracted with EtOAc (2×). The EtOAc extract was washed with brine and concentrated. The residue was purified by column chromatography (0-50% EtOAc in hexane) to get the title compound methyl 5-amino-3-cyano-4-(3-hydroxy-2-methylphenyl)-2-methyl-1-(2,2,2-trifluoroethyl)indole-6-carboxylate (65 mg, 40% yield). LC-MS m/z [M+H]⁺=418.20.

### Step 9: Synthesis of 5-amino-3-cyano-4-(3-hydroxy-2-methylphenyl)-2-methyl-1-(2,2,2-trifluoroethyl)indole-6-carboxamide (compound 61)

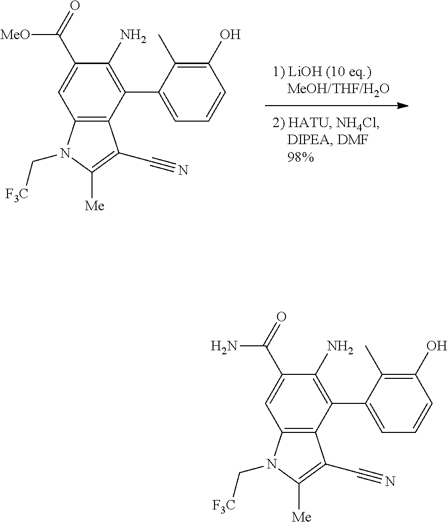 [SMILES: COC(=O)c1cc2c(c(C#N)c(C)n2CC(F)(F)F)c(-c2cccc(O)c2C)c1N ; Cc1c(O)cccc1-c1c(N)c(C(N)=O)cc2c1c(C#N)c(C)n2CC(F)(F)F]

To a solution of methyl 5-amino-3-cyano-4-(3-hydroxy-2-methylphenyl)-2-methyl-1-(2,2,2-trifluoroethyl)indole-6-carboxylate (65 mg, 0.16 mmol) in methanol (2 mL), THF (2 mL), and water (1 mL) was added lithium hydroxide (65 mg, 10 equiv., 1.6 mmol). The mixture was stirred at 60° C. for 3 hours, cooled to room temperature and concentrated. To the residue was added 1N HCl and the mixture was extracted with EtOAc (2×). The EtOAc extract was washed with brine and concentrated. The residue was used directly in the next step without purification. To the solution of crude 5-amino-3-cyano-4-(3-hydroxy-2-methylphenyl)-2-methyl-1-(2,2,2-trifluoroethyl)indole-6-carboxylic acid (62 mg, 0.15 mmol) in DMF (10 ml) was added ammonia hydrochloride (49 mg, 6 equiv., 0.9 mmol) and N-ethyl-N-isopropyl-propan-2-amine (0.16 ml, 6.0 equiv., 0.9 mmol) followed HATU (117 mg, 2.0 equiv., 0.30 mmol, 100 mass %). The reaction mixture was stirred at room temperature for 1 hour, quenched with saturated bicarbonate solution extract with EtOAc (2×). The EtOAc extract was washed with brine and concentrated. The residue was purified by RP-HPLC to get the title compound 5-amino-3-cyano-4-(3-hydroxy-2-methylphenyl)-2-methyl-1-(2,2,2-trifluoroethyl)indole-6-carboxamide (61.6 mg, 98% yield). ¹H NMR (400 MHz, DMSO) δ 9.36 (s, 1H), 8.01 (s, 1H), 7.77 (s, 1H), 7.38 (s, 1H), 7.08 (t, J=7.8 Hz, 1H), 6.86 (dd, J=8.0, 1.2 Hz, 1H), 6.55 (dd, J=7.5, 1.2 Hz, 1H), 5.54 (s, 2H), 5.18 (q, J=9.0 Hz, 2H), 2.48 (s, 3H), 1.78 (s, 3H). LC-MS m/z [M+H]⁺=403.05.

### Example 52. (M)-5-Amino-3-cyano-4-(3-hydroxy-2-methylphenyl)-2-methyl-1-(2,2,2-trifluoroethyl)indole-6-carboxamide (62) and (P)-5-amino-3-cyano-4-(3-hydroxy-2-methylphenyl)-2-methyl-1-(2,2,2-trifluoroethyl)indole-6-carboxamide (63)

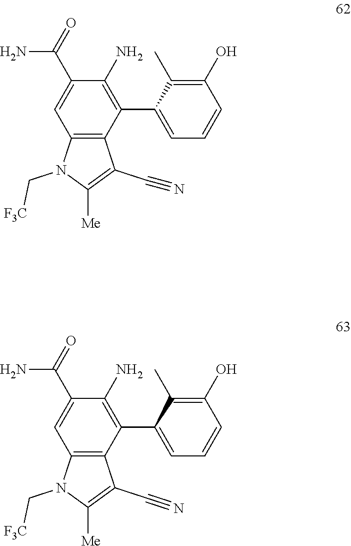

Compounds 62 and 63 were obtained by resolution of compound 61 (40 mg, a racemate). Chiral SFC separation of 5-amino-3-cyano-4-(3-hydroxy-2-methylphenyl)-2-methyl-1-(2,2,2-trifluoroethyl)indole-6-carboxamide (Instrument: Waters SFC Prep 150 Mgm; Column: Daicel Chiralpak OJ, 30×150 mm, 5 um; Conditions: Isocratic at 20% methanol with 80% CO₂; Flow Rate: 100 mL/min) provided Peak 1 (16 mg) and Peak 2 (14 mg).

Peak 1: retention time 2.49 min. ¹H NMR (400 MHz, DMSO) δ 9.36 (s, 1H), 8.01 (s, 1H), 7.77 (s, 1H), 7.38 (s, 1H), 7.08 (t, J=7.8 Hz, 1H), 6.86 (d, J=7.9 Hz, 1H), 6.55 (d, J=7.6 Hz, 1H), 5.54 (s, 2H), 5.18 (q, J=9.1 Hz, 2H), 2.48 (s, 3H), 1.78 (s, 3H). LC-MS m/z [M+H]⁺=403.20.

Peak 2: retention time 2.85 min. ¹H NMR (400 MHz, DMSO) δ 9.31 (s, 1H), 7.94 (s, 1H), 7.70 (s, 1H), 7.31 (s, 1H), 7.01 (t, J=7.8 Hz, 1H), 6.79 (dd, J=8.1, 1.2 Hz, 1H), 6.48 (dd, J=7.6, 1.2 Hz, 1H), 5.47 (s, 2H), 5.11 (q. J=9.0 Hz, 2H), 2.41 (s, 3H), 1.71 (s, 3H). LC-MS m/z [M+H]⁺=403.20.

### Example 53. 5-Amino-3-cyano-4-(3-hydroxy-2-methylphenyl)-1,2-dimethyl-1H-indole-6-carboxamide (64)

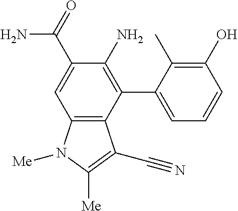 [SMILES: Cc1c(O)cccc1-c1c(N)c(C(N)=O)cc2c1c(C#N)c(C)n2C]

This example was synthesized in the same manner as depicted in Scheme 29, using iodomethane instead of trifluoroethyl triflate as the alkylating agent in step 4. The title compound was obtained. ¹H NMR (400 MHz, DMSO) δ 9.25 (s, 1H), 7.86 (s, 1H), 7.78 (s, 1H), 7.21 (s, 1H), 7.00 (t, J=7.8 Hz, 1H), 6.77 (d, J=8.0 Hz, 1H), 6.52-6.42 (m, 1H), 5.37 (s, 2H), 3.63 (s, 3H), 2.36 (s, 3H), 1.70 (s, 3H). LC-MS m/z [M+H]⁺=335.10.

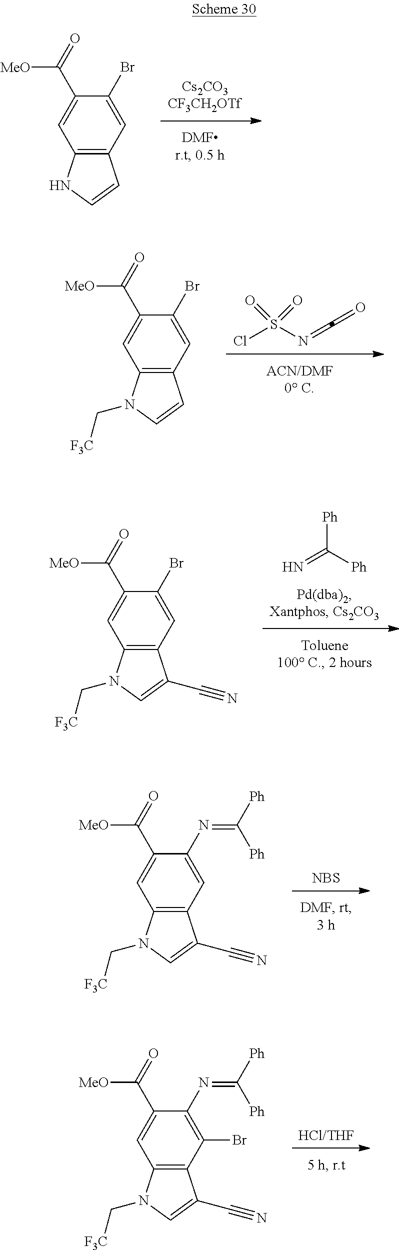

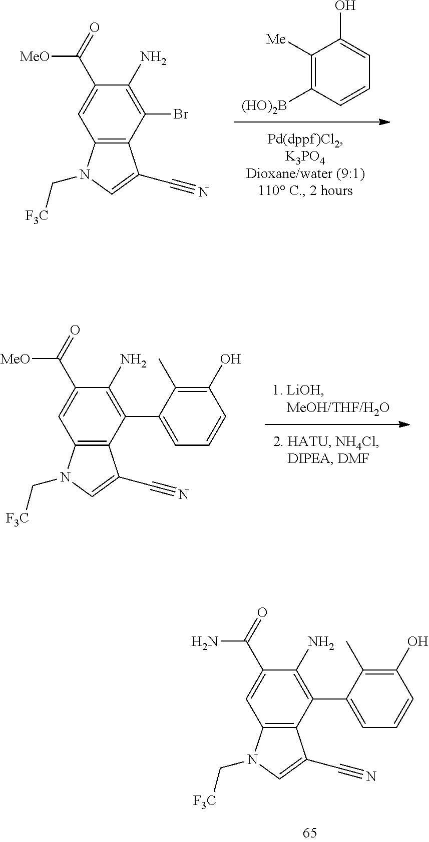 [SMILES: COC(=O)c1cc2c(c(C#N)cn2CC(F)(F)F)c(Br)c1N ; Cc1c(O)cccc1B(O)O ; COC(=O)c1cc2c(c(C#N)cn2CC(F)(F)F)c(-c2cccc(O)c2C)c1N ; Cc1c(O)cccc1-c1c(N)c(C(N)=O)cc2c1c(C#N)cn2CC(F)(F)F]

### Example 54. 5-Amino-3-cyano-4-(3-hydroxy-2-methylphenyl)-1-(2,2,2-trifluoroethyl)-1H-indole-6-carboxamide (65)

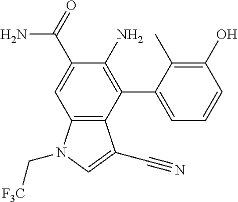 [SMILES: Cc1c(O)cccc1-c1c(N)c(C(N)=O)cc2c1c(C#N)cn2CC(F)(F)F]

### Step 1: Synthesis of methyl 5-bromo-1-(2,2,2-trifluoroethyl)indole-6-carboxylate

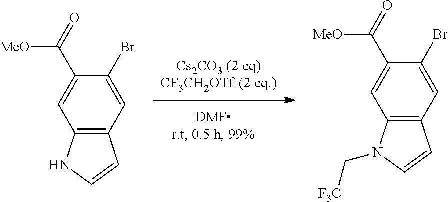 [SMILES: COC(=O)c1cc2[nH]ccc2cc1Br ; COC(=O)c1cc2c(ccn2CC(F)(F)F)cc1Br]

To a solution of methyl 5-bromo-1H-indole-6-carboxylate (500 mg, 2.0 mmol) in DMF (10 ml) was added cesium carbonate (1.3 g, 2.0 equiv., 4.0 mmol) and 2,2,2-trifluoroethyl trifluoromethanesulfonate (0.57 ml, 2.0 equiv., 4.0 mmol). The mixture was stirred at room temperature for 30 min, quenched with water and extracted with EtOAc (2×). The EtOAc extract was washed with brine and concentrated to get the title compound methyl 5-bromo-1-(2,2,2-trifluoroethyl)indole-6-carboxylate which was used in the next step without purification. LC-MS m/z [M+H]⁺=336.00

### Step 2: Synthesis of methyl 5-bromo-3-cyano-1-(2,2,2-trifluoroethyl)indole-6-carboxylate

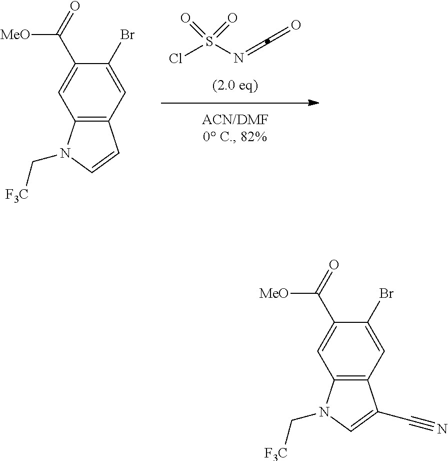

To a solution of methyl 5-bromo-1-(2,2,2-trifluoroethyl)indole-6-carboxylate (660 mg, 1.96 mmol) in anhydrous acetonitrile (10 mL) was added N-(oxomethylene)sulfamoyl chloride (0.34 ml, 2.0 equiv., 3.92 mmol) dropwise at 0° C. The resulting reaction mixture was stirred at 0° C. for 2 hours, after which DMF (5 mL %) was added dropwise. The mixture was stirred at 0° C. for 1 hour, quenched with water and extracted with EtOAc (2×). The EtOAc extract was washed with brine and concentrated. The residue was purified by column chromatography (0-20% EtOAc in hexane) to give the title compound methyl 5-bromo-3-cyano-1-(2,2,2-trifluoroethyl)indole-6-carboxylate (600 mg, 82% yield). LC-MS m/z [M+H]⁺=361.00

### Step 3: Synthesis of methyl 5-(benzhydrylideneamino)-3-cyano-1-(2,2,2-trifluoroethyl)indole-6-carboxylate

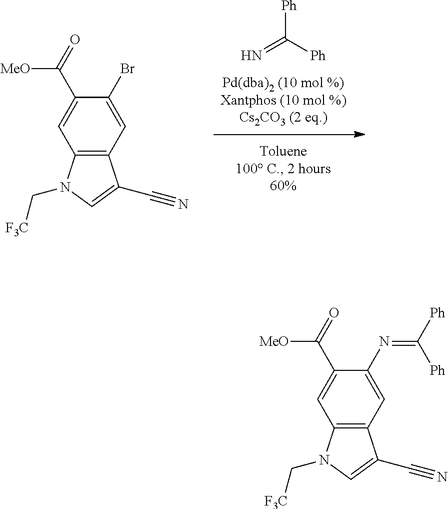 [SMILES: COC(=O)c1cc2c(cc1Br)c(C#N)cn2CC(F)(F)F ; N=C(c1ccccc1)c1ccccc1 ; COC(=O)c1cc2c(cc1N=C(c1ccccc1)c1ccccc1)c(C#N)cn2CC(F)(F)F]

To a solution of methyl 5-bromo-3-cyano-1-(2,2,2-trifluoroethyl)indole-6-carboxylate (700 mg, 1.9 mmol) and diphenylmethanimine (0.65 ml, 2.0 equiv., 3.8 mmol) in toluene (15 mL) was added cesium carbonate (1.27 g, 2.0 equiv., 3.8 mmol), Pd₂(dba)₃ (177 mg, 0.1 equiv., 0.19 mmol), and Xantphos (112 mg, 0.1 equiv., 0.19 mmol). The mixture was stirred at 100° C. for 2 hours, cooled to room temperature, quenched with water, and extracted with EtOAc (2×). The EtOAc extract was washed with brine and concentrated. The residue was purified by column chromatography (0-20% EtOAc in hexane) to give the title compound methyl 5-(benzhydrylideneamino)-3-cyano-1-(2,2,2-trifluoroethyl)indole-6-carboxylate (480 mg, 60% yield). LC-MS m/z [M+H]⁺=462.10.

### Step 4: Synthesis of methyl 5-(benzhydrylideneamino)-4-bromo-3-cyano-1-(2,2,2-trifluoroethyl)indole-6-carboxylate

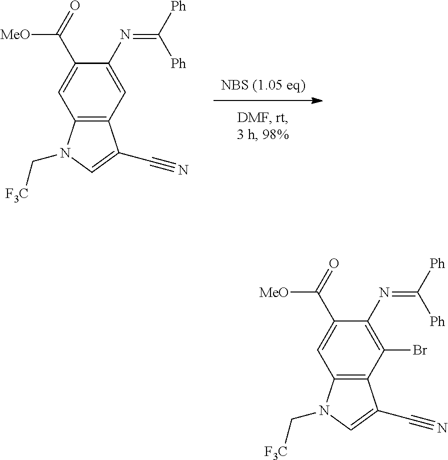 [SMILES: COC(=O)c1cc2c(cc1N=C(c1ccccc1)c1ccccc1)c(C#N)cn2CC(F)(F)F ; "NBS (1.05 eq)" ; COC(=O)c1cc2c(c(C#N)cn2CC(F)(F)F)c(Br)c1N=C(c1ccccc1)c1ccccc1]

To a solution of methyl 5-(benzhydrylideneamino)-3-cyano-1-(2,2,2-trifluoroethyl)indole-6-carboxylate (710 mg, 1.5 mmol) in DMF (5 ml) was added 1-bromopyrrolidine-2,5-dione (NBS, 300 mg, 1.05 equiv., 1.59 mmol). The mixture was stirred at room temperature for 3 hours, quenched with water and extracted with EtOAc (2×). The EtOAc extract was washed with brine and concentrated to give the title compound methyl 5-(benzhydrylideneamino)-4-bromo-3-cyano-1-(2,2,2-trifluoroethyl)indole-6-carboxylate which was used in the next step without purification. LC-MS m/z [M+H]⁺=542.05.

### Step 5: Synthesis of methyl 5-amino-4-bromo-3-cyano-1-(2,2,2-trifluoroethyl)indole-6-carboxylate

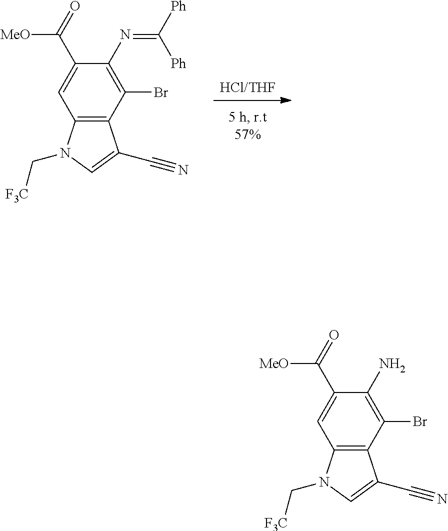 [SMILES: COC(=O)c1cc2c(c(C#N)cn2CC(F)(F)F)c(Br)c1N=C(c1ccccc1)c1ccccc1 ; "HCl/THF" ; COC(=O)c1cc2c(c(C#N)cn2CC(F)(F)F)c(Br)c1N]

To a solution of methyl 5-(benzhydrylideneamino)-4-bromo-3-cyano-1-(2,2,2-trifluoroethyl)indole-6-carboxylate (300 mg, 0.56 mmol) in THF (2 ml) was added hydrochloric acid (2 mL). The mixture was stirred at room temperature for 5 hours, quenched with water and extracted with EtOAc (2×). The EtOAc extract was washed with brine and concentrated. The residue was purified by column chromatography (0-50% EtOAc in hexane) to give the title compound methyl 5-amino-4-bromo-3-cyano-1-(2,2,2-trifluoroethyl)indole-6-carboxylate (120 mg, 57% yield). LC-MS m/z [M+H]⁺=376.

### Step 6: Synthesis of methyl 5-amino-3-cyano-4-(3-hydroxy-2-methylphenyl)-1-(2,2,2-trifluoroethyl)indole-6-carboxylate

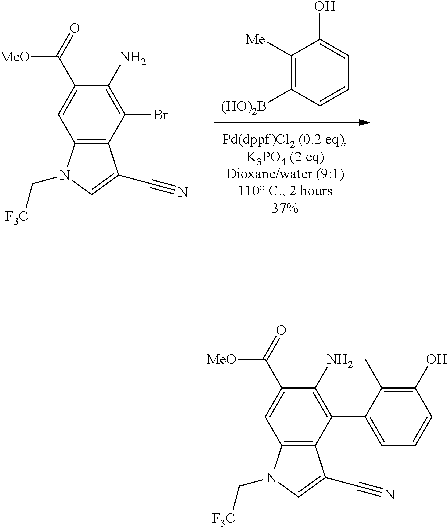 [SMILES: COC(=O)c1cc2c(c(C#N)cn2CC(F)(F)F)c(Br)c1N ; Cc1c(O)cccc1B(O)O ; COC(=O)c1cc2c(c(C#N)cn2CC(F)(F)F)c(-c2cccc(O)c2C)c1N]

To a solution of methyl 5-amino-4-bromo-3-cyano-1-(2,2,2-trifluoroethyl)indole-6-carboxylate (100 mg, 0.27 mmol), potassium phosphate (112 mg, 2.0 equiv., 0.54 mmol) and (3-hydroxy-2-methylphenyl)boronic acid (81 mg, 2.0 equiv., 0.54 mmol) in the 1,4-dioxane (2 mL) and water (0.2 mL) was added the Pd(dppf)Cl₂ (39 mg, 0.2 equiv., 0.054 mmol) under nitrogen. The mixture was stirred at 110° C. for 2 hours, cooled to room temperature, quenched with water, and extracted with EtOAc (2×). The EtOAc extract was washed with brine and concentrated. The residue was purified by column chromatography (0-100% EtOAc in hexane) to give the title compound methyl 5-amino-3-cyano-4-(3-hydroxy-2-methylphenyl)-1-(2,2,2-trifluoroethyl)indole-6-carboxylate (40 mg, 37% yield). LC-MS m/z [M+H]⁺=404.15.

### Step 7: Synthesis of 5-amino-3-cyano-4-(3-hydroxy-2-methylphenyl)-1-(2,2,2-trifluoroethyl)indole-6-carboxamide (compound 65)

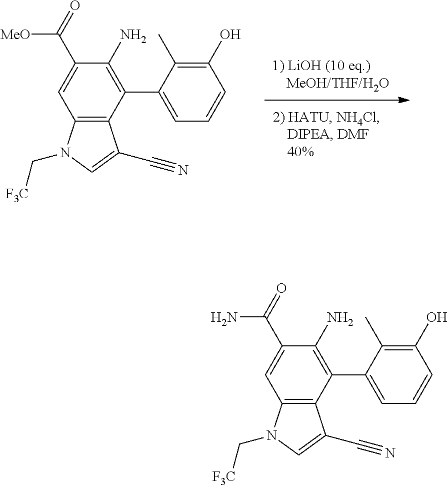 [SMILES: COC(=O)c1cc2c(c(C#N)cn2CC(F)(F)F)c(-c2cccc(O)c2C)c1N ; Cc1c(O)cccc1-c1c(N)c(C(N)=O)cc2c1c(C#N)cn2CC(F)(F)F]

To a solution of methyl 5-amino-3-cyano-4-(3-hydroxy-2-methylphenyl)-1-(2,2,2-trifluoroethyl)indole-6-carboxylate (40 mg, 0.1 mmol) in methanol (2 mL), THF (2 mL), and water (1 mL) was added lithium hydroxide (42 mg, 10 equiv., 1.0 mmol). The mixture was stirred at 60° C. for 3 hours, cooled to room temperature and concentrated. To the resulting residue was added 1N HCl (aq.) and the mixture was extracted with EtOAc (2×). The EtOAc extract was washed with brine and concentrated. The residue was used directly in the next step without further purification. To a solution of crude 5-amino-3-cyano-4-(3-hydroxy-2-methylphenyl)-1-(2,2,2-trifluoroethyl)indole-6-carboxylic acid (38 mg, 0.1 mmol) in DMF (2 ml) was added ammonium chloride (31 mg, 6.0 equiv., 0.6 mmol), N-ethyl-N-isopropyl-propan-2-amine (0.1 ml, 6.0 equiv., 0.6 mmol), and HATU (74 mg, 2.0 equiv., 0.2 mmol). The reaction mixture was stirred at room temperature for 1 hour, quenched with saturated bicarbonate solution and extracted with EtOAc (2×). The EtOAc extract was washed with brine and concentrated. The residue was purified by RP-HPLC to give the title compound 5-amino-3-cyano-4-(3-hydroxy-2-methylphenyl)-1-(2,2,2-trifluoroethyl)indole-6-carboxamide (15 mg, 40% yield). ¹H NMR (400 MHz, DMSO) δ 9.31 (s, 1H), 8.15 (s, 1H), 8.02 (s, 1H), 7.75 (s, 1H), 7.37 (s, 1H), 7.02 (t, J=7.8 Hz, 1H), 6.83-6.76 (m, 1H), 6.53-6.47 (m, 1H), 5.43 (s, 2H), 5.10 (q, J=9.1 Hz, 2H), 1.71 (s, 3H). LC-MS m/z [M+H]⁺=389.10.

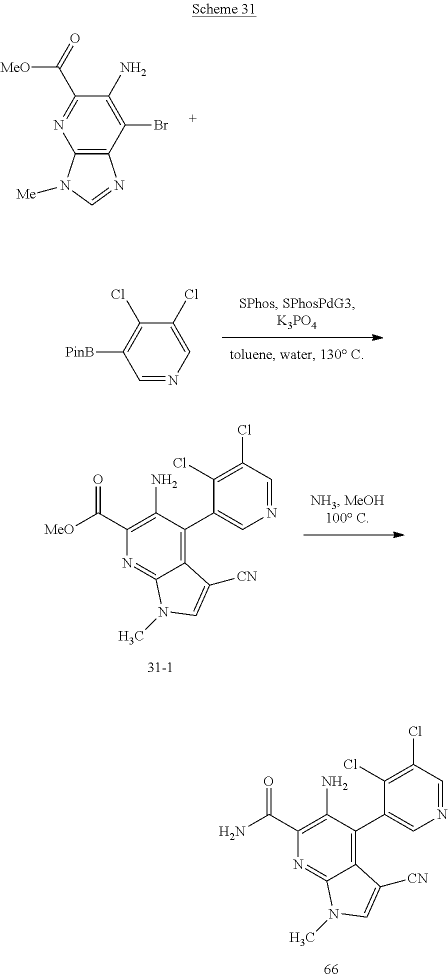

### Example 55. 5-Amino-3-cyano-4-(4,5-dichloropyridin-3-yl)-1-methyl-1H-pyrrolo[2,3-b]pyridine-6-carboxamide (66)

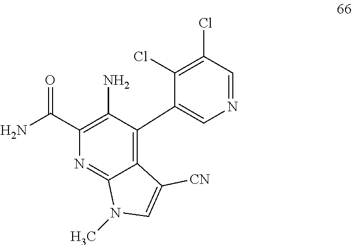 [SMILES: Cn1cc(C#N)c2c(-c3cncc(Cl)c3Cl)c(N)c(C(N)=O)nc21]

### Step 1: Methyl 5-amino-3-cyano-4-(4,5-dichloropyridin-3-yl)-1-methyl-1H-pyrrolo[2,3-b]pyridine-6-carboxylate

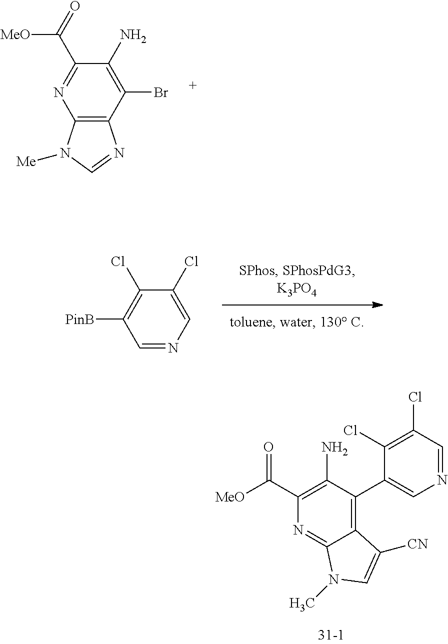

Methyl 5-amino-4-bromo-3-cyano-1-methyl-pyrrolo[2,3-b]pyridine-6-carboxylate (50 mg, 0.12 mmol, 76% purity, 1 equiv.), 3,4-dichloro-5-(4,4,5,5-tetramethyl-1,3,2-dioxaborolan-2-yl)pyridine (130 mg, 0.50 mmol, 4 equiv.), K₃PO₄ (100 mg, 0.50 mmol, 4 equiv.), and SPhos (20 mg, 0.050 mmol, 0.4 equiv.) were added to a microwave vial. Toluene (1.8 mL) and water (0.2 mL) were added, the flask was sparged with nitrogen gas, SPhosPdG₃ (40 mg, 0.050 mmol, 0.4 equiv.) was added, the flask was sparged with nitrogen gas and sealed. The vial was heated to 130° C. for 5 hr. The reaction mixture was poured onto Celite® then subjected to FCC (Hexanes:EtOAc:MeOH, 100:0:0 to 0:100:0 to 0:80:20) to obtain the title compound that was used directly in the next step.

### Step 2: Synthesis of 5-amino-3-cyano-4-(4,5-dichloropyridin-3-yl)-1-methyl-1H-pyrrolo[2,3-b]pyridine-6-carboxamide (Compound 66)

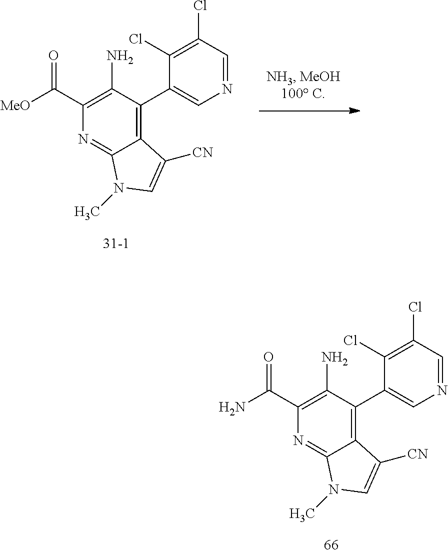 [SMILES: COC(=O)c1nc2c(c(C#N)cn2C)c(-c2cncc(Cl)c2Cl)c1N ; Cn1cc(C#N)c2c(-c3cncc(Cl)c3Cl)c(N)c(C(N)=O)nc21]

Crude methyl 5-amino-3-cyano-4-(4,5-dichloropyridin-3-yl)-1-methyl-1H-pyrrolo[2,3-b]pyridine-6-carboxylate (0.12 mmol theoretical) was suspended in an ammonia solution in MeOH (7 M, 5 mL), and heated in a microwave reactor at 100° C. for 1 hr. The solvent was removed. The residue was dissolved in minimal DMF, filtered, and subjected to RP-HPLC (0.1% FA, Water:MeCN 90:10 to 0:100) to obtain the title compound (11 mg, 24% yield over 2 steps). ¹H NMR (400 MHz, DMSO) δ 8.84 (s, 1H), 8.44 (s, 1H), 8.37 (s, 1H), 8.30 (s, 1H), 7.56 (s, 1H), 6.54 (s, 2H), 3.82 (s, 3H). LC-MS m/z 361 (M+H⁺).

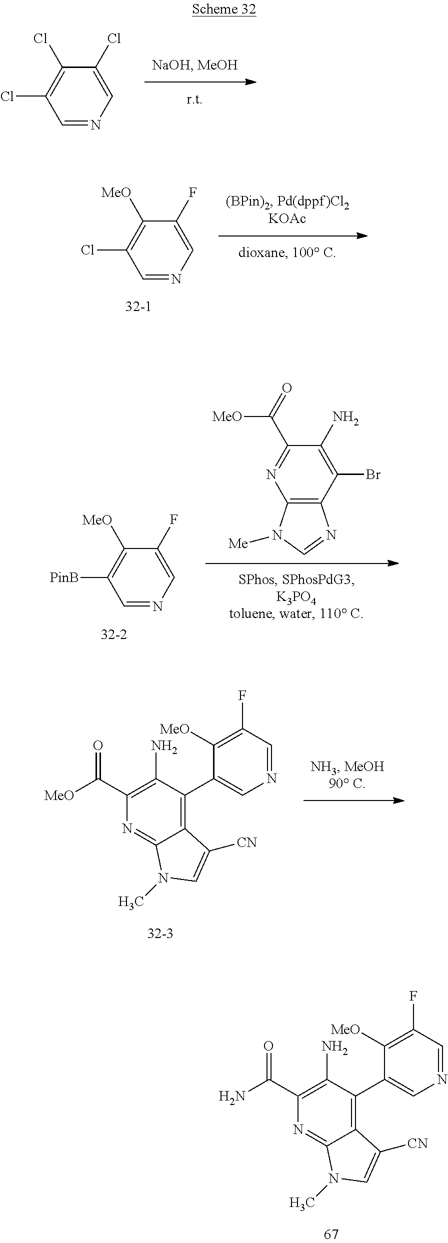

### Example 56. 5-Amino-3-cyano-4-(5-fluoro-4-methoxypyridin-3-yl)-1-methyl-1H-pyrrolo[2,3-b]pyridine-6-carboxamide. (67)

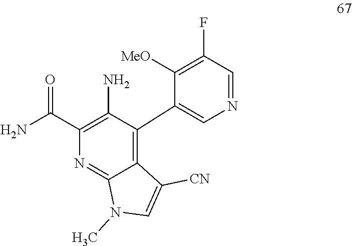 [SMILES: COc1c(F)cncc1-c1c(N)c(C(N)=O)nc2c1c(C#N)cn2C]

### Step 1: 3-Chloro-5-fluoro-4-methoxypyridine

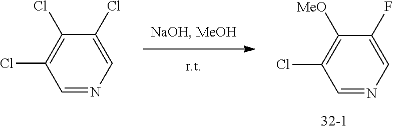 [SMILES: Clc1cncc(Cl)c1Cl ; COc1c(F)cncc1Cl]

NaOMe (2.2 g, 40 mmol, 4 equiv.) was dissolved in MeOH (20 mL). While still warm, 3-chloro-4,5-difluoro-pyridine (1.5 g, 10 mmol, 1 equiv.) was added and the mixture was stirred for 1 hr at room temperature. The reaction mixture was poured into a saturated solution of NaHCO₃ (75 mL), diluted with water (50 mL), and extracted with DCM (2×100 mL). The organic layers were combined, dried over MgSO₄, filtered, and the solvent removed to obtain the title compound (1.62 g, crude) of sufficient purity for the next step.

### Step 2: 3-Fluoro-4-methoxy-5-(4,4,5,5-tetramethyl-1,3,2-dioxaborolan-2-yl)pyridine

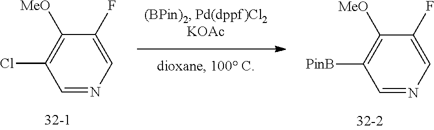

3-Chloro-5-fluoro-4-methoxy-pyridine (1.62 g, 10.0 mmol, 1 equiv.), bis(pinacolato)diboron (3.81 g, 15.0 mmol, 1.5 equiv.), and potassium acetate (1.47 g, 15.0 mmol, 1.5 equiv.) were added to 1,4-dioxane (10 mL). The mixture was sparged with nitrogen gas, Pd(dppf)Cl₂ (732 mg, 1.00 mmol, 0.1 equiv.) was added, the mixture was sealed, then heated in a microwave reactor at 100° C. for 1 hr. The reaction mixture was diluted with DCM (20 mL), filtered through Celite®, rinsed with DCM, and the solvent removed. The residue was adsorbed to Celite® and subjected to FCC (Hexanes:EtOAc, 100:0 to 0:100) to obtain the title compound (432 mg, 17% over 2 steps) of sufficient purity for the next step.

### Step 3: Methyl 5-amino-3-cyano-4-(5-fluoro-4-methoxypyridin-3-yl)-1-methyl-1H-pyrrolo[2,3-b]pyridine-6-carboxylate

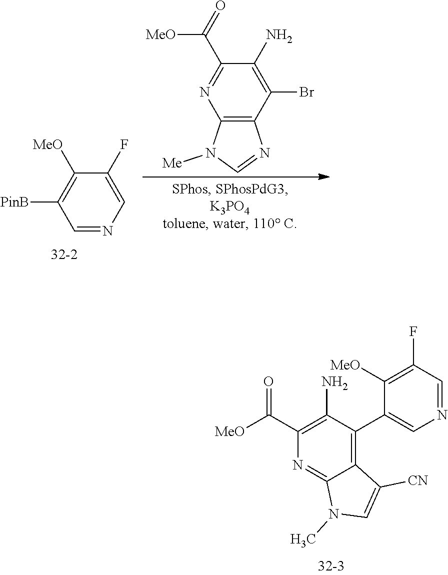

Methyl 5-amino-4-bromo-3-cyano-1-methyl-pyrrolo[2,3-b]pyridine-6-carboxylate (50.0 mg, 0.123 mmol, 76% purity, 1 equiv.), 3-fluoro-4-methoxy-5-(4,4,5,5-tetramethyl-1,3,2-dioxaborolan-2-yl)pyridine (124 mg, 0.492 mmol, 4 equiv.), K₃PO₄ (104 mg, 0.492 mmol, 4 equiv.), and SPhos (20 mg, 0.049 mmol, 0.4 equiv.) were added to a microwave vial. Toluene (0.9 mL) and water (0.1 mL) were added, the flask was sparged with nitrogen gas, and SPhosPdG₃ (38 mg, 0.049 mmol, 0.4 equiv.) was added. The mixture was sealed and heated in a microwave reactor to 110° C. for 2 hr. The reaction mixture was poured onto Celite® then subjected to FCC (Hexanes:EtOAc:MeOH, 100:0:0 to 0:100:0 to 0:80:20) to obtain the title compound (59 mg, crude) of sufficient purity for the next step.

### Step 4: 5-Amino-3-cyano-4-(5-fluoro-4-methoxypyridin-3-yl)-1-methyl-1H-pyrrolo[2,3-b]pyridine-6-carboxamide (Compound 67)

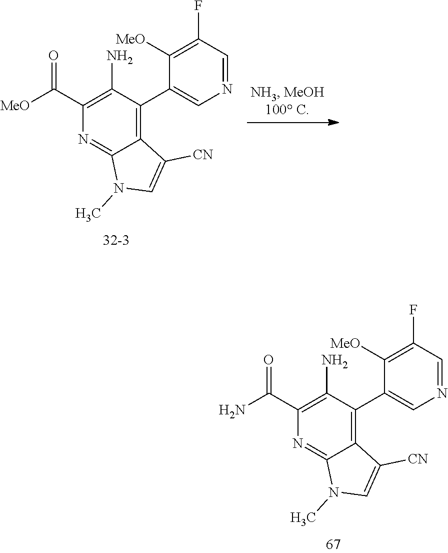 [SMILES: COC(=O)c1nc2c(c(C#N)cn2C)c(-c2cncc(F)c2OC)c1N ; COc1c(F)cncc1-c1c(N)c(C(N)=O)nc2c1c(C#N)cn2C]

Crude methyl 5-amino-3-cyano-4-(5-fluoro-4-methoxypyridin-3-yl)-1-methyl-1H-pyrrolo[2,3-b]pyridine-6-carboxylate (59 mg) was suspended in an ammonia solution in MeOH (7 M, 4 mL), and heated to 90° C. in a microwave reactor for 3 hour. The solvent was removed, the residue was dissolved in DMF, filtered, and subjected to RP-HPLC (0.1% FA, Water:MeCN 90:10 to 0:100) to obtain the title compound (11 mg, 26% over 2 steps). ¹H NMR (400 MHz, DMSO) δ 8.57 (d, J=4.4 Hz, 1H), 8.35 (s, 1H), 8.28 (d, J=2.9 Hz, 1H), 8.09 (s, 1H), 7.53 (d, J=2.9 Hz, 1H), 6.40 (s, 2H), 3.85 (d, J=4.1 Hz, 3H), 3.81 (s, 3H). ¹⁹F NMR (376 MHz, DMSO) δ−146.84 (p, J=4.3 Hz). LC-MS m/z 341 (M+H⁺).

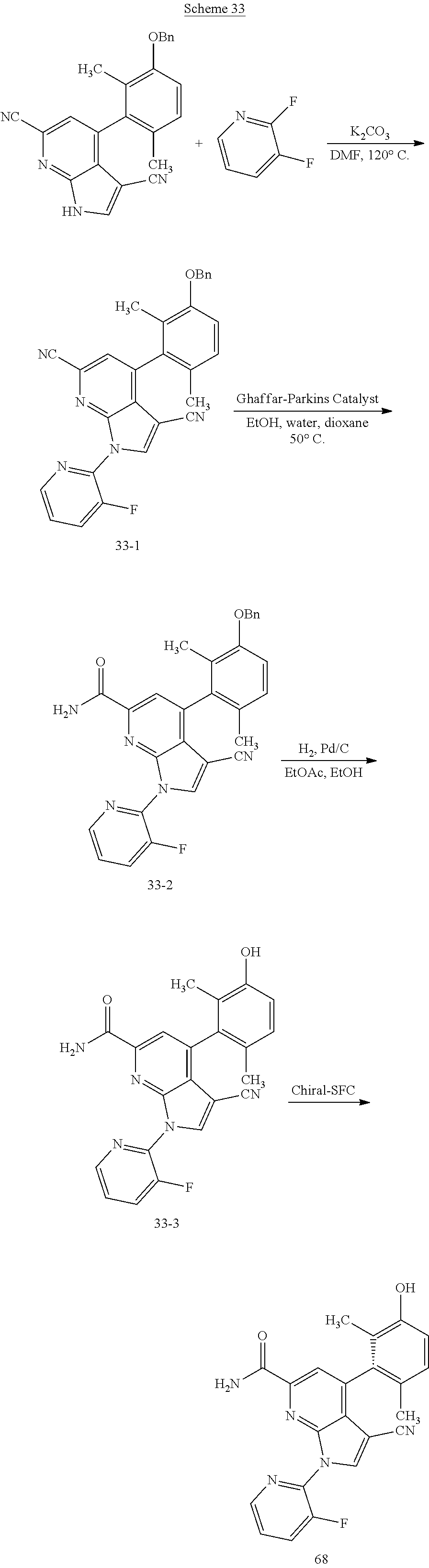

### Example 57. (P)-3-Cyano-1-(3-fluoropyridin-2-yl)-4-(3-hydroxy-2,6-dimethylphenyl)-1H-pyrrolo[2,3-b]pyridine-6-carboxamide. (68)

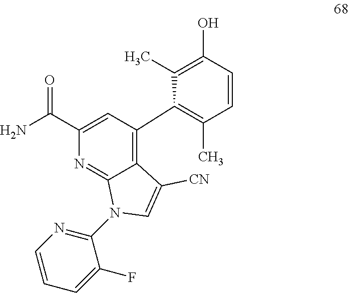

### Step 1: 4-(3-(Benzyloxy)-2,6-dimethylphenyl)-1-(3-fluoropyridin-2-yl)-1H-pyrrolo[2,3-b]pyridine-3,6-dicarbonitrile

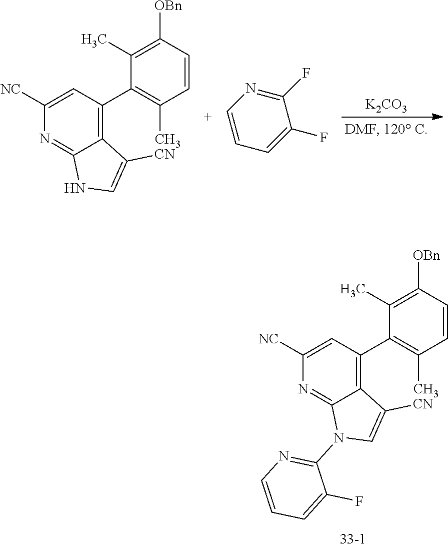

4-(3-Benzyloxy-2,6-dimethyl-phenyl)-1H-pyrrolo[2,3-b]pyridine-3,6-dicarbonitrile (204 mg, 0.540 mmol, 1 equiv.) and potassium carbonate (446 mg, 3.24 mmol, 6 equiv.) were added to DMF (4 mL) in a 4 mL vial, 2,3-difluoropyridine (0.296 mL, 3.24 mmol, 6 equiv.) was added and the mixture was stirred at 120° C. overnight. The mixture was diluted with DCM, filtered through Celite®, and rinsed with DCM. The solvent was removed, the residue adsorbed to Celite®, and subjected to FCC (Hexanes:EtOAc, 100:0 to 0:100) to obtain the title compound (161 mg, 63%).

### Step 2: 4-(3-(Benzyloxy)-2,6-dimethylphenyl)-3-cyano-1-(3-fluoropyridin-2-yl)-1H-pyrrolo[2,3-b]pyridine-6-carboxamide

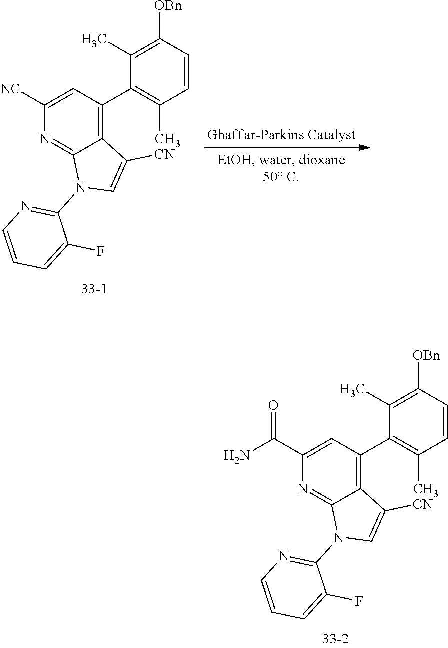 [SMILES: Cc1ccc(OCc2ccccc2)c(C)c1-c1cc(C#N)nc2c1c(C#N)cn2-c1ncccc1F ; Cc1ccc(OCc2ccccc2)c(C)c1-c1cc(C(N)=O)nc2c1c(C#N)cn2-c1ncccc1F]

4-(3-Benzyloxy-2,6-dimethyl-phenyl)-1-(3-fluoro-2-pyridyl)pyrrolo[2,3-b]pyridine-3,6-dicarbonitrile (160 mg, 0.338 mmol, 1 equiv.) was dissolved in ethanol (2 mL), 1,4-dioxane (2 mL), and water (1 mL). Ghaffar-Parkins catalyst (15 mg, 0.034 mmol, 0.1 equiv.) was added and the mixture was heated to 50° C. for 3 hr. The solvent was removed, the solid was adsorbed to Celite®, and subjected to FCC (Hexanes:DCM:MeOH, 100:0:0 to 0:100:0 to 0:95:5) to obtain the title compound (111 mg, 67%).

### Step 3: 3-Cyano-1-(3-fluoropyridin-2-yl)-4-(3-hydroxy-2,6-dimethylphenyl)-1H-pyrrolo[2,3-b]pyridine-6-carboxamide

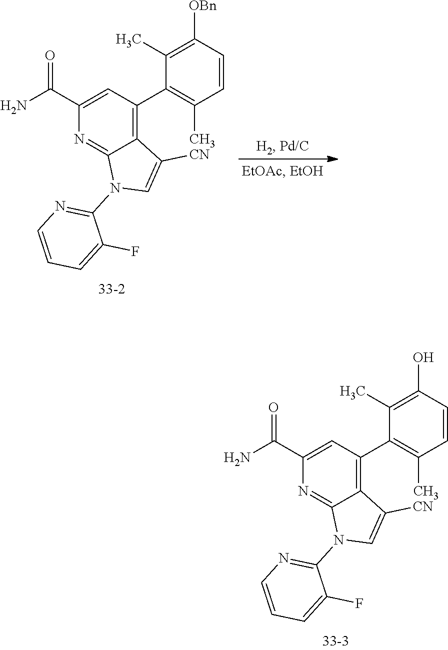 [SMILES: Cc1ccc(OCc2ccccc2)c(C)c1-c1cc(C(N)=O)nc2c1c(C#N)cn2-c1ncccc1F ; Cc1ccc(O)c(C)c1-c1cc(C(N)=O)nc2c1c(C#N)cn2-c1ncccc1F]

4-(3-Benzyloxy-2,6-dimethyl-phenyl)-3-cyano-1-(3-fluoro-2-pyridyl)pyrrolo[2,3-b]pyridine-6-carboxamide (110 mg, 0.224 mmol, 1 equiv.) was dissolved in ethyl acetate (2 mL) and ethanol (2 mL) and sparged with nitrogen gas. Palladium on carbon (10%, 50 mg) was added, the mixture was sparged with hydrogen gas and stirred overnight at room temperature under a balloon of hydrogen gas. The mixture was sparged with nitrogen gas, filtered, rinsed with EtOAc, then the solvent removed to obtain the title compound (112 mg, crude) of sufficient purity for the next step.

### Step 4: (S)-3-Cyano-1-(3-fluoropyridin-2-yl)-4-(3-hydroxy-2,6-dimethylphenyl)-1H-pyrrolo[2,3-b]pyridine-6-carboxamide (Compound 68)

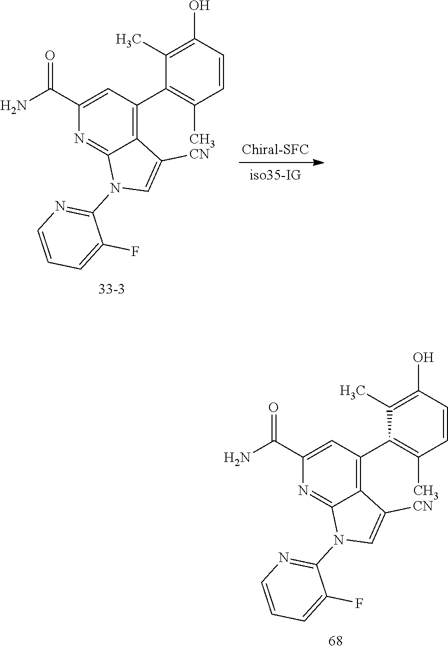

Chiral SFC separation of crude 3-cyano-1-(3-fluoropyridin-2-yl)-4-(3-hydroxy-2,6-dimethylphenyl)-1H-pyrrolo[2,3-b]pyridine-6-carboxamide (112 mg) (Instrument: Waters SFC Prep 150 Mgm; Column: Daicel Chiralpak IG, 30×150 mm, 5 um; Conditions: Isocratic at 35% methanol with 65% CO₂: Flow Rate: 100 mL/min) provided compound 68 (24 mg, 27% yield). LCMS: m/z 402 [M+H]⁺; ¹H NMR (400 MHz, DMSO) δ 9.34 (s, 1H), 8.98 (s, 1H), 8.56 (d, J=4.7 Hz, 1H), 8.18 (t, J=9.2 Hz, 1H), 7.89-7.66 (m, 4H), 7.01 (d, J=8.2 Hz, 1H), 6.85 (d, J=8.2 Hz, 1H), 1.83 (s, 3H), 1.76 (s, 3H). ¹⁹F NMR (376 MHz, DMSO) δ −122.96 (dd, J=9.9, 4.0 Hz)

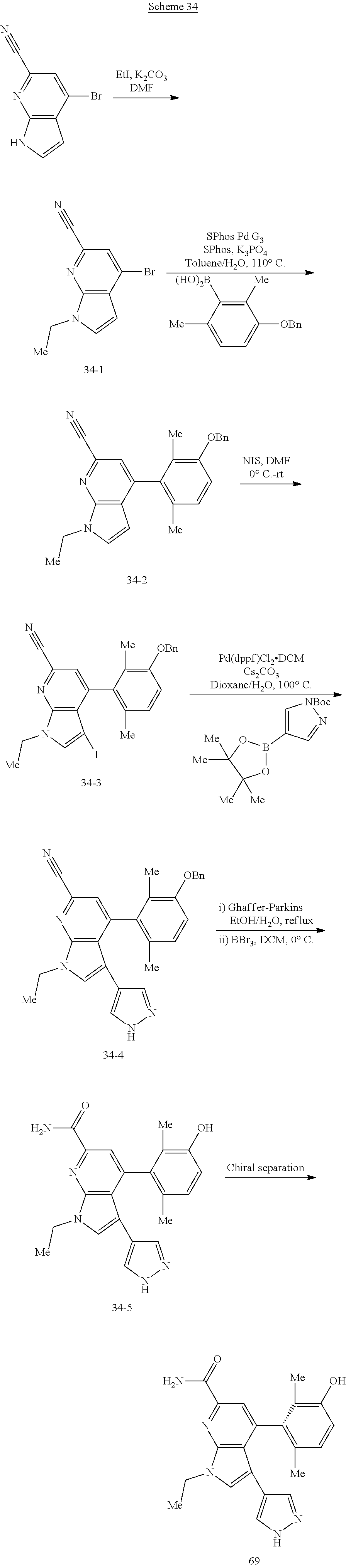

### Example 58. (P)-1-Ethyl-4-(3-hydroxy-2,6-dimethylphenyl)-3-(1H-pyrazol-4-yl)-1H-pyrrolo[2,3-b]pyridine-6-carboxamide. (69)

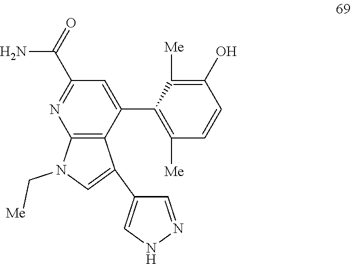

### Step 1: 4-Bromo-1-ethyl-1H-pyrrolo[2,3-b]pyridine-6-carbonitrile

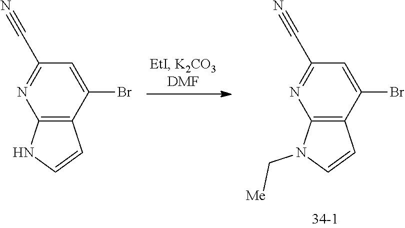 [SMILES: N#Cc1cc(Br)c2cc[nH]c2n1 ; CCn1ccc2c(Br)cc(C#N)nc21]

4-Bromo-1H-pyrrolo[2,3-b]pyridine-6-carbonitrile (750 mg, 3.38 mmol, 1.00 equiv.) was dissolved in DMF (7.5 mL) under nitrogen. Potassium carbonate (820 mg, 5.93 mmol, 1.75 equiv.) followed by iodoethane (0.47 mL, 5.90 mmol, 1.75 equiv.) was added and the reaction was stirred at room temperature for 30 mins. The reaction was diluted with H₂O (40 mL) and a precipitate formed, and this was stirred while cooling in an ice bath for 30 mins. The solid precipitate was collected by vacuum filtration and then washed with H₂O (3×20 mL). The material was dried thoroughly to yield the title compound (0.734 g, 87%). LC-MS m/z 250 (M+H⁺).

### Step 2: 4-(3-(Benzyloxy)-2,6-dimethylphenyl)-1-ethyl-1H-pyrrolo[2,3-b]pyridine-6-carbonitrile

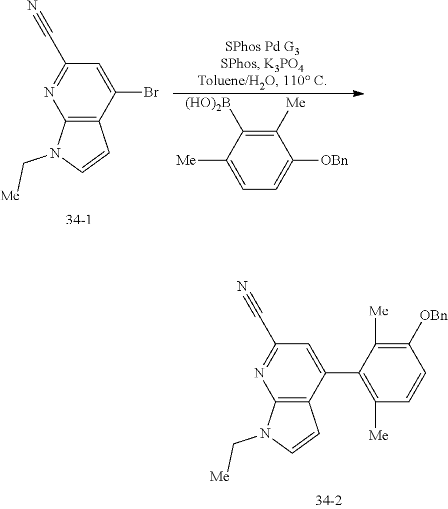 [SMILES: CCn1ccc2c(Br)cc(C#N)nc21 ; Cc1ccc(OCc2ccccc2)c(C)c1B(O)O ; CCn1ccc2c(-c3c(C)ccc(OCc4ccccc4)c3C)cc(C#N)nc21]

4-Bromo-1-ethyl-TH-pyrrolo[2,3-b]pyridine-6-carbonitrile (638 mg, 2.55 mmol, 1.00 equiv.), (3-(benzyloxy)-2,6-dimethylphenyl)boronic acid (1.10 g, 4.30 mmol, 1.70 equiv.), dicyclohexyl(2′,6′-dimethoxy[1,1′-biphenyl]-2-yl)phosphane (SPhos, 210 mg, 0.511 mmol, 0.20 equiv.), and K₃PO₄ (1.35 g, 6.36 mmol, 2.50 equiv.) were suspended in toluene (13 mL) and water (0.3 mL). The solution was sparged with N₂ for 2 mins, then (2-dicyclohexylphosphino-2′,6′-dimethoxybiphenyl) [2-(2′-amino-1,1′-biphenyl)]palladium(II) methanesulfonate (SPhos Pd G3, 400 mg, 0.511 mmol, 0.20 equiv.) was added, and the reaction was sparged for another 5 minutes. The reaction was then heated in the microwave to 110° C. for 1.5 hours. The solvent was removed and the residue was adsorbed onto silica and then subjected to FCC (EtOAc:Hexanes 0:100 to 35:65) to afford the title compound (990 mg, 101% yield). LC-MS m/z 382 (M+H⁺).

### Step 3: 4-(3-(Benzyloxy)-2,6-dimethylphenyl)-1-ethyl-3-iodo-1H-pyrrolo[2,3-b]pyridine-6-carbonitrile

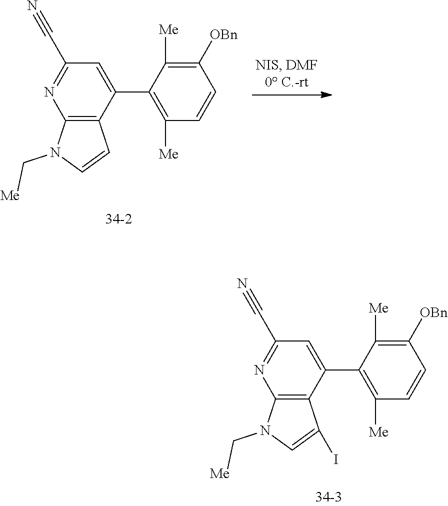 [SMILES: CCn1ccc2c(-c3c(C)ccc(OCc4ccccc4)c3C)cc(C#N)nc21 ; CCn1cc(I)c2c(-c3c(C)ccc(OCc4ccccc4)c3C)cc(C#N)nc21]

4-(3-(Benzyloxy)-2,6-dimethylphenyl)-1-ethyl-1H-pyrrolo[2,3-b]pyridine-6-carbonitrile (943 mg, 2.55 mmol, 1.00 equiv.) was dissolved in N,N-dimethylformamide (12 mL) under nitrogen and then cooled to 0° C. 1-Iodopyrrolidine-2,5-dione (NIS, 574 mg, 2.55 mmol, 1.00 equiv.) was added and the reaction was allowed to warm to room temperature for 2 hours. The reaction mixture was diluted with EtOAc (50 mL) and H₂O (60 mL) and the layers were separated. The organic layer was collected, and the aqueous layer was extracted with EtOAc (3×). The organic layers were combined and dried with Na₂SO₄, filtered, and the solvent was removed. The residue was adsorbed onto silica and then subjected to FCC (EtOAc:Hexanes 0:100 to 40:60) to afford the title compound (940 mg, 72% yield). LC-MS m/z 508 (M+H⁺).

### Step 4: 4-(3-(Benzyloxy)-2,6-dimethylphenyl)-1-ethyl-3-(1H-pyrazol-4-yl)-1H-pyrrolo[2,3-b]pyridine-6-carbonitrile

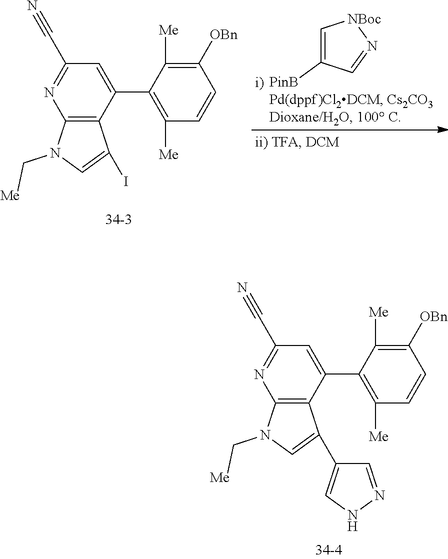

4-(3-(Benzyloxy)-2,6-dimethylphenyl)-1-ethyl-3-iodo-1H-pyrrolo[2,3-b]pyridine-6-carbonitrile (262 mg, 0.516 mmol, 1.00 equiv.), [1,1′-bis(diphenylphosphino)ferrocene]dichloropalladium(II), complex with dichloromethane (36 mg, 0.044 mmol, 0.085 equiv.) cesium carbonate (428 mg, 1.31 mmol, 2.5 equiv.), tert-butyl 4-(4,4,5,5-tetramethyl-1,3,2-dioxaborolan-2-yl)-1H-pyrazole-1-carboxylate (321 mg, 1.09 mmol, 2.11 equiv.) were combined and suspended in 1,4-dioxane (3 mL) and H₂O (0.3 mL). The solution was sparged with N₂ for 5 mins, then heated to 100° C. for 7 hours. The reaction was diluted with EtOAc (50 mL) and H₂O (60 mL) and the layers were separated. The organic layer was collected, and the aqueous layer was extracted EtOAc (3×). The organic layers were combined, washed brine (3×), dried with Na₂SO₄, filtered, and the solvent was removed. The resulting residue was dissolved in 30 v/v % TFA in DCM (5 mL) and the solution stirred at room temperature for 30 minutes. The solvent was removed, the residue was adsorbed onto silica, and subjected to FCC (EtOAc:Hexanes 20:80 to 100:00) to afford the title compound (111 mg, 40% yield). LC-MS m/z 448 (M+H⁺).

### Step 5: 1-Ethyl-4-(3-hydroxy-2,6-dimethylphenyl)-3-(1H-pyrazol-4-yl)-1H-pyrrolo[2,3-b]pyridine-6-carboxamide

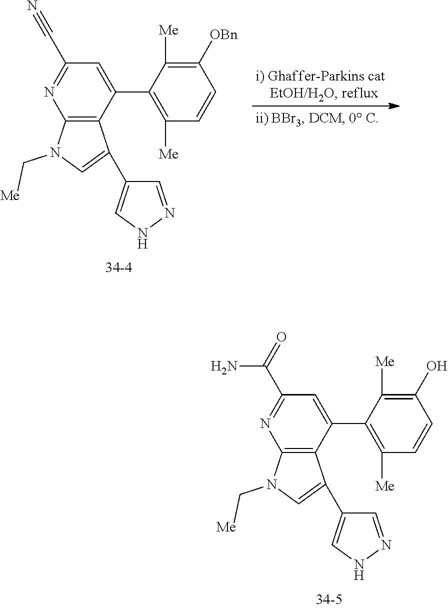 [SMILES: CCn1cc(-c2cn[nH]c2)c2c(-c3c(C)ccc(OCc4ccccc4)c3C)cc(C#N)nc21 ; CCn1cc(-c2cn[nH]c2)c2c(-c3c(C)ccc(O)c3C)cc(C(N)=O)nc21]

4-(3-(Benzyloxy)-2,6-dimethylphenyl)-1-ethyl-3-(1H-pyrazol-4-yl)-1H-pyrrolo[2,3-b]pyridine-6-carbonitrile (111 mg, 0.25 mmol, 1.00 equiv.) and Ghaffar-Parkins catalyst (21 mg, 0.049 mmol, 0.200 equiv.) were suspended in ethanol (7 mL) and H₂O (3 mL) and heated to reflux for 12 hours. The solvent was removed, EtOAc (50 mL) and H₂O (50 mL) were added, and the layers were separated. The organic layer was collected, and the aqueous layer was further extracted with EtOAc (×3). The organic layers were combined, washed brine (3×), and then dried with Na₂SO₄, filtered, and the solvent was removed to give an off-white solid. The solid was dissolved in DCM (1.4 mL) under nitrogen and cooled to ° C. A solution of BBr₃ in DCM (1 M, 1.0 mL, 1.0 mmol, 4.0 equiv.) was added dropwise over 1 min and the mixture was stirred 0° C. for 1 hour. The reaction was diluted with DCM (3 mL) and then quenched with sat NaHCO₃ (5 mL). EtOAc (40 mL) and H₂O (40 mL) were added and the layers were separated. The organic layer was collected, and the aqueous layer was further extracted with EtOAc (3×). The organic layers were combined, washed with brine (3×), dried with Na₂SO₄, filtered, and the solvent was removed. The residue was subjected to RP-HPLC (0.1% FA, Water:MeCN, 95:5 to 0:100) to afford the title compound (14 mg, 15% yield). ¹H NMR (400 MHz, DMSO) δ 9.22 (s, 1H), 8.23 (s, 1H), 7.95 (s, 1H), 7.58 (s, 1H), 7.41 (s, 1H), 6.88 (d, J=8.2 Hz, 1H), 6.78 (d, J=8.2 Hz, 2H), 4.47 (q. J=7.2 Hz, 2H), 1.67 (s, 3H), 1.60 (s, 3H), 1.49 (t, J=7.2 Hz, 3H). LC-MS m/z 376 (M+H⁺).

### Step 6: (P)-1-Ethyl-4-(3-hydroxy-2,6-dimethylphenyl)-3-(1H-pyrazol-4-yl)-1H-pyrrolo[2,3-b]pyridine-6-carboxamide. (69)

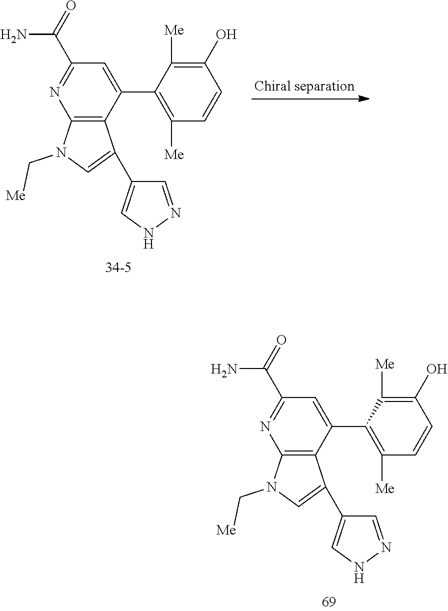

1-Ethyl-4-(3-hydroxy-2,6-dimethylphenyl)-3-(1H-pyrazol-4-yl)-1H-pyrrolo[2,3-b]pyridine-6-carboxamide was subjected to purification by chiral SFC (Waters UPCC, Chiralpak AD-3), peak 2 (rt=1.558 minutes) was collected and dried to the title compound (38.7 mg). ¹H NMR (400 MHz, DMSO) δ 12.38 (s, 1H), 9.19 (s, 1H), 8.21 (s, 1H), 7.94 (s, 1H), 7.55 (s, 1H), 7.41 (s, 1H), 6.87 (d, J=8.2 Hz, 1H), 6.77 (d, J=8.2 Hz, 2H), 4.65 (q, J=7.2 Hz, 2H), 1.68 (s, 3H), 1.61 (s, 3H), 1.49 (t, J=7.2 Hz, 3H). LC-MS m/z 376 (M+H⁺).

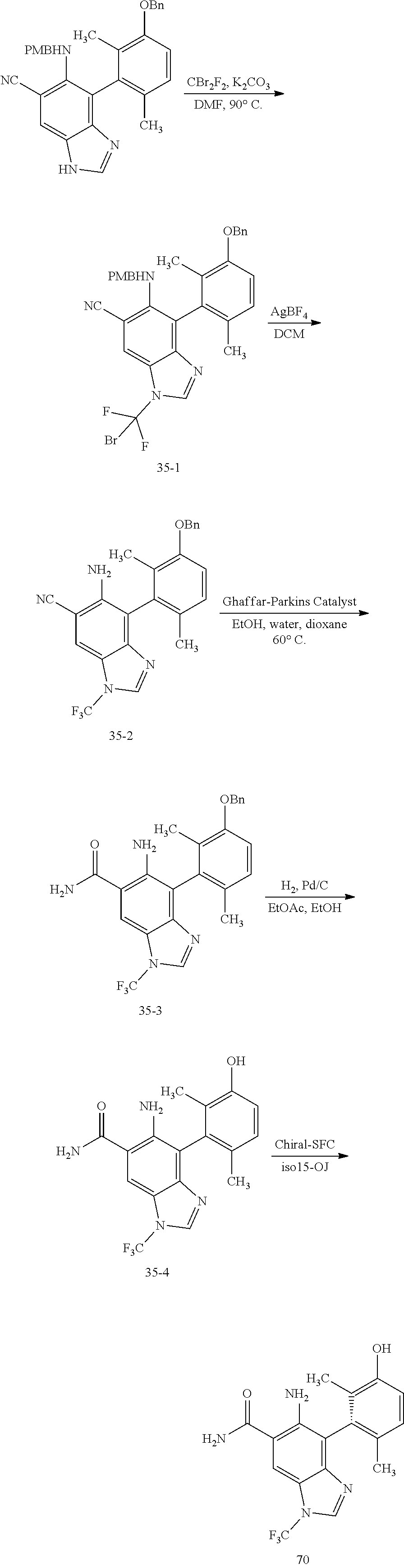

### Example 59. (P)-5-Amino-4-(3-hydroxy-2,6-dimethylphenyl)-1-(trifluoromethyl)-1H-benzo[d]imidazole-6-carboxamide. (70)

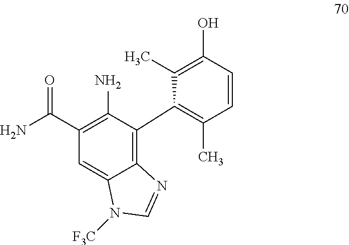

### Step 1: 4-(3-(Benzyloxy)-2,6-dimethylphenyl)-1-(bromodifluoromethyl)-5-((4-methoxybenzyl)amino)-1H-benzo[d]imidazole-6-carbonitrile

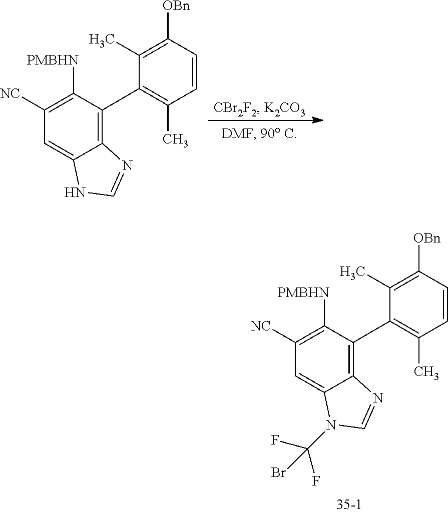

7-(3-Benzyloxy-2,6-dimethyl-phenyl)-6-[(4-methoxyphenyl)methylamino]-3H-benzimidazole-5-carbonitrile (4.47 g, 9.15 mmol, 1 equiv.) was dissolved in DMF (10 mL) in a 20 mL microwave vessel containing potassium carbonate (2.53 g, 18.3 mmol, 2 equiv.). The mixture was sparged with nitrogen gas, dibromodifluoromethane (6.7 mL, 73 mmol, 8 equiv.) was added, and the mixture was heated in a microwave reactor to 90° C. for 30 min. The reaction mixture was filtered, the solvent was removed, the material was adsorbed to Celite®, and subjected to FCC (Hexanes:EtOAc, 100:0 to 40:60) to obtain the title compound (2.48 g, 44%).

### Step 2: 5-Amino-4-(3-(benzyloxy)-2,6-dimethylphenyl)-1-(trifluoromethyl)-1H-benzo[d]imidazole-6-carbonitrile

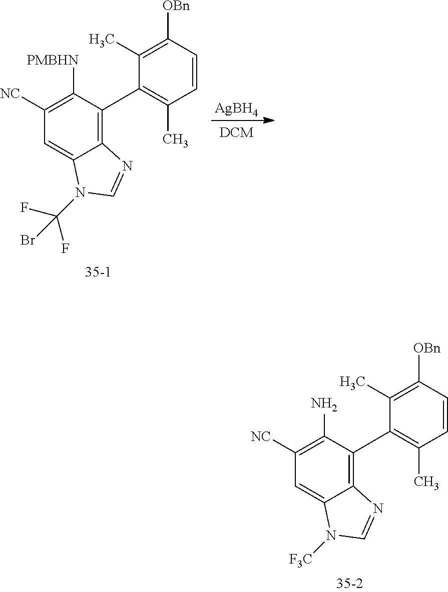

7-(3-Benzyloxy-2,6-dimethyl-phenyl)-3-[bromo(difluoro)methyl]-6-[(4-methoxyphenyl)methylamino]benzimidazole-5-carbonitrile (1.89 g, 3.07 mmol, 1 equiv.) was dissolved in DCM (20 mL), sparged with nitrogen gas, and cooled to 0° C. Anisole (1.0 mL, 9.2 mmol, 3 equiv.) was added, followed by silver tetrafluoroborate (2.98 g, 15.3 mmol, 5 equiv.) and the mixture was stirred in the dark overnight allowing the ice bath to expire. A further 2.5 equiv. of AgBF₄ (1.49 g) was added and the mixture was stirred at room temperature for 3 hr. The mixture was poured into saturated aqueous solution of NaHCO₃ (100 mL) and extracted with DCM (2×100 mL). The organic layers were combined and brine (50 mL) was added. The mixture was filtered through Celite®) and the organic layer was separated, dried over MgSO₄, and the solvent removed. The residue was taken up in minimal DCM and subjected to FCC (Hexanes:EtOAc, 100:0 to 20:80) to obtain the title compound (945 mg, 71%).

### Step 3: 5-Amino-4-(3-(benzyloxy)-2,6-dimethylphenyl)-1-(trifluoromethyl)-1H-benzo[d]imidazole-6-carboxamide

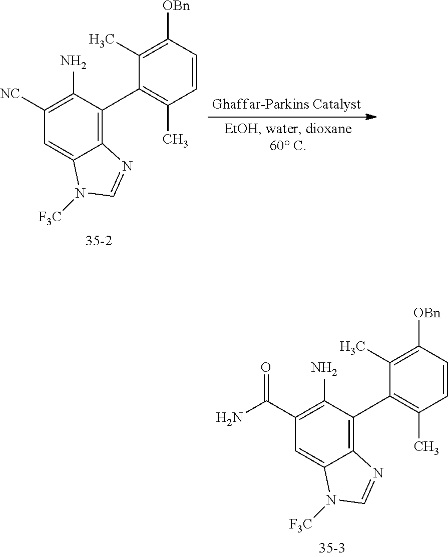 [SMILES: Cc1ccc(OCc2ccccc2)c(C)c1-c1c(N)c(C#N)cc2c1ncn2C(F)(F)F ; Cc1ccc(OCc2ccccc2)c(C)c1-c1c(N)c(C(N)=O)cc2c1ncn2C(F)(F)F]

6-Amino-7-(3-benzyloxy-2,6-dimethyl-phenyl)-3-(trifluoromethyl)benzimidazole-5-carbonitrile (294 mg, 0.674 mmol, 1 equiv.) was dissolved in 1,4-dioxane (5 mL) and ethanol (5 mL). Water (5 mL) then Ghaffar-Parkins catalyst (29 mg, 0.067 mmol, 0.1 equiv.) were added and the mixture was heated to 60° C. for 1 hr. The solvent was removed, the residue was taken up in minimal DCM with trace MeOH, and subjected to FCC (Hexanes:DCM:MeOH, 50:50:0 to 0:100:0 to 0:95:5) to obtain the title compound (303 mg, 99%).

### Step 4: 5-Amino-4-(3-hydroxy-2,6-dimethylphenyl)-1-(trifluoromethyl)-1H-benzo[d]imidazole-6-carboxamide

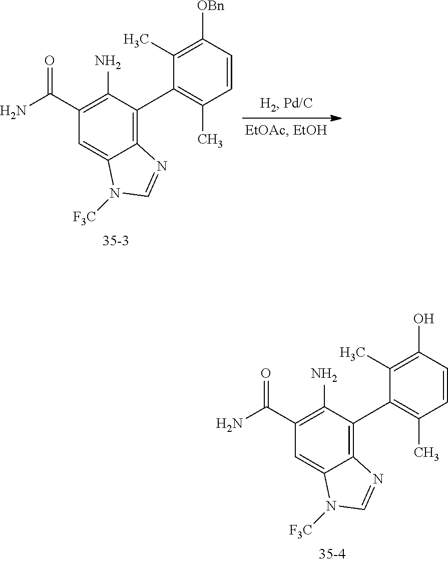 [SMILES: Cc1ccc(OCc2ccccc2)c(C)c1-c1c(N)c(C(N)=O)cc2c1ncn2C(F)(F)F ; Cc1ccc(O)c(C)c1-c1c(N)c(C(N)=O)cc2c1ncn2C(F)(F)F]

6-Amino-7-(3-benzyloxy-2,6-dimethyl-phenyl)-3-(trifluoromethyl)benzimidazole-5-carboxamide (2.22 g, 4.89 mmol, 1 equiv.) was dissolved in ethyl acetate (50 mL) and ethanol (50 mL). The mixture was sparged with nitrogen gas. Palladium on carbon (10%, 1.25 g) was added, the mixture was sparged with hydrogen gas, then stirred under a balloon of hydrogen gas for 75 min. The mixture was sparged with nitrogen gas. Celite® was added, the mixture was filtered through a pad of Celite®) rinsed with EtOAc/EtOH (1:1, 50 mL then 20 mL), and the solvent removed. The residue was dissolved in minimal DCM with trace MeOH and subjected to FCC (DCM:MeOH, 100:0 to 80:20) to obtain the title compound (1.45 g, 82%).

### Step 5: (P)-5-Amino-4-(3-hydroxy-2,6-dimethylphenyl)-1-(trifluoromethyl)-1H-benzo[d]imidazole-6-carboxamide (70)

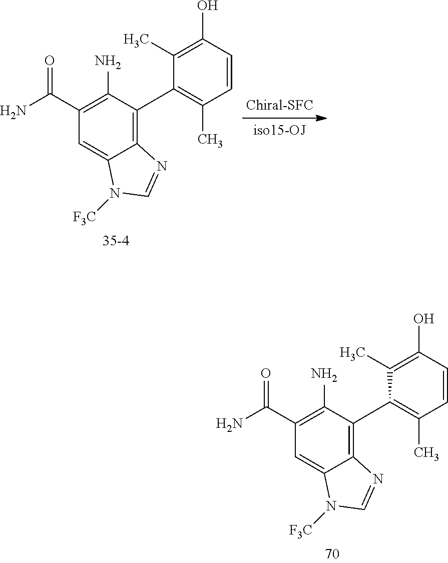

Chiral SFC separation of 5-amino-4-(3-hydroxy-2,6-dimethylphenyl)-1-(trifluoromethyl)-1H-benzo[d]imidazole-6-carboxamide (1.45 g) (Instrument: Waters SFC Prep 150 Mgm; Column: Daicel Chiralpak IG, 30×150 mm, 5 um; Conditions: Isocratic at 15% methanol with 85% CO₂: Flow Rate: 100 mL/min) provided compound 70 (555 mg, 33% yield). LCMS: m/z 365 [M+H]⁺; ¹H NMR (400 MHz, DMSO) δ 9.17 (s, 1H), 8.62 (s, 1H), 8.17 (s, 1H), 7.88 (s, 1H), 7.41 (s, 1H), 6.97 (d, J=8.1 Hz, 1H), 6.79 (d, J=8.1 Hz, 1H), 5.58 (s, 2H), 1.75 (s, 3H), 1.69 (s, 3H). ¹⁹F NMR (376 MHz, DMSO) δ −55.83.

### Example 60. (P)-1-Ethyl-4-(3-hydroxy-2,6-dimethylphenyl)-3-(1,2,4-oxadiazol-3-yl)-1H-pyrrolo[2,3-b]pyridine-6-carboxamide. (71)

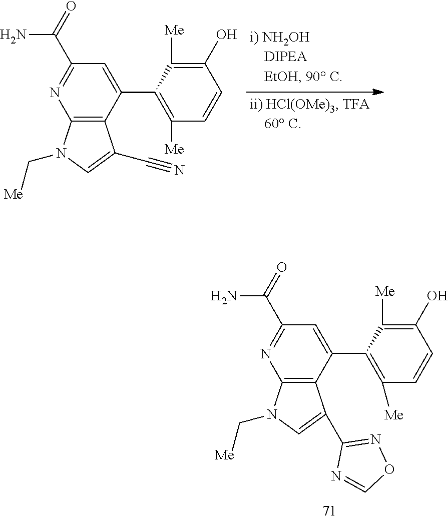

(P)-3-Cyano-1-ethyl-4-(3-hydroxy-2,6-dimethylphenyl)-1H-pyrrolo[2,3-b]pyridine-6-carboxamide (50.0 mg, 0.150 mmol, 1.00 equiv.) was dissolved in ethanol (0.35 mL) and N,N-diisopropylethylamine (DIPEA, 0.16 mL, 0.89 mmol, 6 equiv.). Hydroxylamine (50% solution in H₂O, 0.20 mL, 3.0 mmol, 20 equiv.) was added and the reaction was heated to 90° C. in the microwave for 24 hours. The solvent was removed. The residue was suspended in trimethyl orthoformate (1.5 mL) and TFA (0.05 mL) and the reaction was heated to 60° C. for 30 minutes. The solvent was removed, and the residue was subjected to RP-HPLC (0.1% FA, Water:MeCN, 95:5 to 0:100) to afford the title compound (2.4 mg, 4% yield). ¹H NMR (400 MHz, DMSO) δ 9.25 (s, 1H), 9.01 (s, 1H), 8.40 (s, 1H), 8.31 (s, 1H), 7.68 (d, J=2.8 Hz, 1H), 7.53 (s, 1H), 6.77 (d, J=8.2 Hz, 1H), 6.68 (d, J=8.2 Hz, 1H), 4.55 (q, J=7.2 Hz, 2H), 1.66 (s, 3H), 1.60 (s, 3H), 1.51 (t, J=7.2 Hz, 3H). LC-MS m/z 378 (M+H⁺).

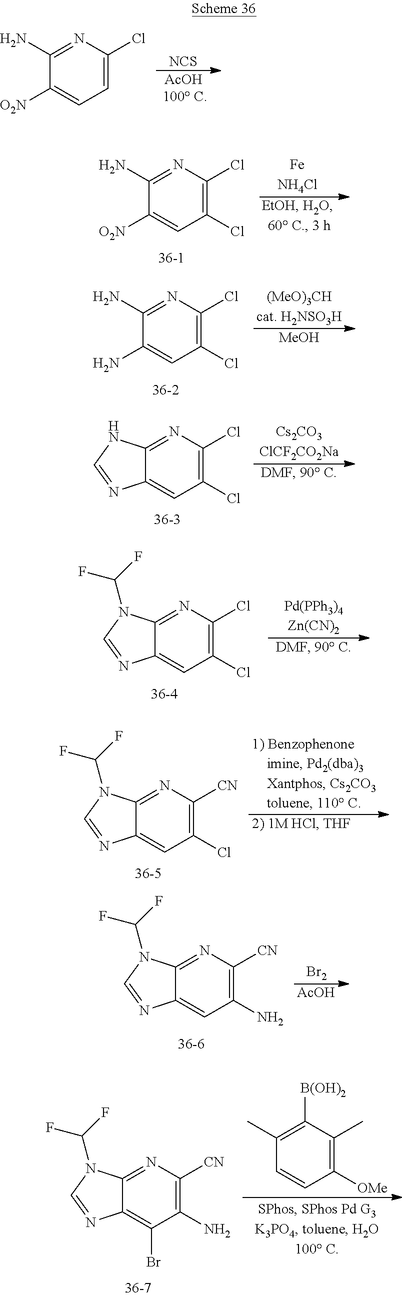

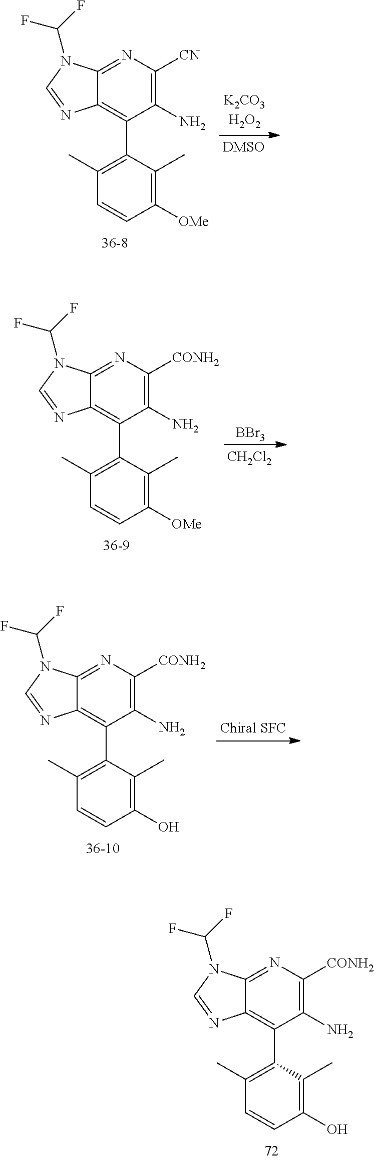

### Example 61. (P)-6-Amino-3-(difluoromethyl)-7-(3-hydroxy-2,6-dimethylphenyl)-3H-imidazo[4,5-b]pyridine-5-carboxamide (72)

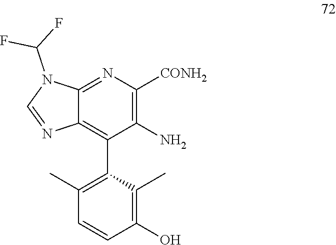

### Step 1: 5,6-Dichloro-3-nitropyridin-2-amine

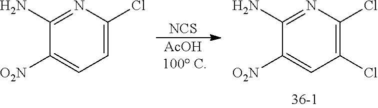 [SMILES: Nc1nc(Cl)ccc1[N+](=O)[O-] ; "AcOH 100° C." ; "NCS" ; Nc1nc(Cl)c(Cl)cc1[N+](=O)[O-]]

6-Chloro-3-nitro-pyridin-2-amine (5 g, 28.8 mmol, 1.0 equiv) and N-chlorosuccinimide (NCS, 1.05 equiv) were suspended in glacial acetic acid (50 mL). The reaction mixture was heated to 100° C. for 90 min. A further 0.1 equiv of NCS was added, the mixture was heated for 1 h, and the solvent was evaporated in vacuo. To the residue was added water (50 mL) and the pH was adjusted to 7-8 with saturated NaHCO₃. The resulting precipitate was isolated by filtration and dried under vacuum to give the title compound (5.54 g, 93% yield). LC-MS m/z 208 (M+H⁺).

### Step 2: 5,6-Dichloropyridine-2,3-diamine

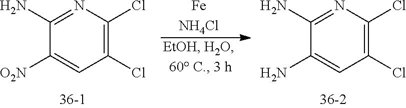 [SMILES: Nc1nc(Cl)c(Cl)cc1[N+](=O)[O-] ; "FeNH4Cl" ; "EtOH, H2O, 60° C., 3 h" ; Nc1cc(Cl)c(Cl)nc1N]

A mixture of 5,6-dichloro-3-nitro-pyridin-2-amine (5.54 g, 26.6 mmol, 1.0 equiv.), iron powder (7.44 g, 133.2 mmol, 5.0 equiv.), ammonium chloride (7.12 g, 133.1 mmol, 5.0 equiv.), ethanol (120 mL) and water (47 mL) heated to 60° C. for 3 hr. The mixture was cooled to room temperature, filtered through Celite®, and washed with EtOH. The combined filtrate was concentrated in vacuo to remove most of the EtOH. The remaining dark purple aqueous solution was extracted with EtOAc. The organic layer was dried over MgSO₄, filtered, and concentrated to give the title compound (4.41 g, 93% yield). LC-MS m/z 178 (M+H⁺).

### Step 3: 5,6-Dichloro-3H-imidazo[4,5-b]pyridine

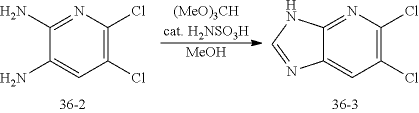 [SMILES: "MeOH" ; Nc1cc(Cl)c(Cl)nc1N ; "(MeO)3CHcat. H2NSO3H" ; Clc1cc2nc[nH]c2nc1Cl]

5,6-Dichloropyridine-2,3-diamine (4.41 g, 24.8 mmol, 1.0 equiv.), methanol (130 mL), trimethoxymethane (4.2 mL, 38 mmol, 1.5 equiv.) and sulfamic acid (120 mg, 1.24 mmol, 0.05 equiv.) were stirred overnight at room temperature. The reaction mixture was concentrated, the residue was triturated with hexane (50 mL), and filtered to isolate the title compound (3.60 g, 77% yield) as a gray powder. LC-MS m/z 188 (M+H⁺).

### Step 4: 5,6-Dichloro-3-(difluoromethyl)-3H-imidazo[4,5-b]pyridine

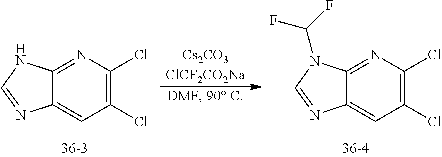 [SMILES: Clc1cc2nc[nH]c2nc1Cl ; "Cs2CO3ClCF2CO2Na" ; "DMF, 90° C." ; FC(F)n1cnc2cc(Cl)c(Cl)nc21]

A round bottom flask was charged with 5,6-dichloro-3H-imidazo[4,5-b]pyridine (100 mg, 0.53 mmol, 1.0 equiv.), sodium 2-chloro-2,2-difluoro-acetate (162 mg, 1.06 mmol, 2.0 equiv.) and Cs₂CO₃ (347 mg, 1.07 mmol, 2.0 equiv.). The reaction mixture was evacuated and refilled with nitrogen gas. Anhydrous DMF (5 mL) was then added, and the mixture was stirred at 90° C. for 3 hr. The reaction mixture was cooled to room temperature, diluted with saturated aqueous NH₄Cl, and extracted with EtOAc. The organic layer was dried over MgSO₄, filtered, and concentrated. The residue was adsorbed onto silica gel and subjected to FCC (2-80% EtOAc in hexanes) to afford the title compound (31 mg, 24% yield). LC-MS m/z 238 (M+H⁻).

### Step 5: 6-chloro-3-(difluoromethyl)-3H-imidazo[4,5-b]pyridine-5-carbonitrile

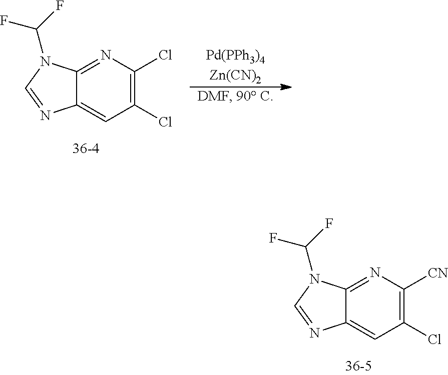 [SMILES: FC(F)n1cnc2cc(Cl)c(Cl)nc21 ; "DMF, 90° C." ; N#Cc1nc2c(cc1Cl)ncn2C(F)F ; "Pd(PPh3)4Zn(CN)2"]

A round bottom flask was charged with 5,6-dichloro-3-(difluoromethyl)imidazo[4,5-b]pyridine (442 mg, 1.86 mmol, 1 equiv.), Pd(PPh₃)₄ (429 mg, 0.37 mmol, 0.2 equiv.) and Zn(CN)₂ (131 mg, 1.12 mmol, 0.6 equiv.), then evacuated and refilled with nitrogen gas. Anhydrous DMF (18 mL) was added and the mixture was stirred at 90° C. for 1 hr. A further 0.05 equiv. of Pd(PPh₃)₄ and 0.2 eq Zn(CN)₂ were added, and the mixture was stirred at 90° C. for 1 hr. Reaction mixture was concentrated, the residue was suspended in water, and filtered. The aqueous filtrate was extracted with EtOAc, the organic layer was combined with the filtered solid, and concentrated in vacuo. The residue was adsorbed onto silica gel and purified by FCC (5-80% EtOAc in hexanes) to give the title compound (300 mg, 71% yield). LC-MS m/z 229 (M+H⁺).

### Step 6: 6-Amino-3-(difluoromethyl)-3H-imidazo[4,5-b]pyridine-5-carbonitrile

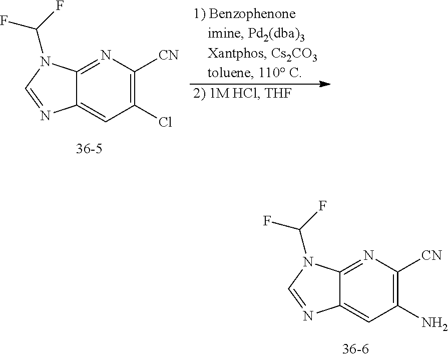

A sealed tube was charged with 6-chloro-3-(difluoromethyl)imidazo[4,5-b]pyridine-5-carbonitrile (125 mg, 0.55 mmol, 1 equiv.), Xantphos (127 mg, 0.22 mmol, 0.4 equiv.), Pd₂(dba)₃ (100 mg, 0.11 mmol, 0.2 equiv.), Cs₂CO₃ (356 mg, 1.09 mmol, 2.0 equiv.) and benzophenone imine (129 mg, 0.71 mmol, 1.3 equiv.). The reaction tube was evacuated and refilled with nitrogen (2×). Toluene sparged with nitrogen (4.6 mL) was added, and the tube was sealed and stirred at 120° C. for 16 hr. After cooling to room temperature, the mixture was diluted with EtOAc and filtered through Celite®. The filter cake was washed with additional EtOAc. and the combined filtrate was concentrated. The residue was re-dissolved in THF (5 mL) and aqueous HCl (1 M, 1.3 mL) was added. The mixture was stirred for 3 hr before basifying with a saturated aqueous solution of NaHCO₃ to pH 9 and extracting with EtOAc. The organic layer was dried over MgSO₄, filtered, and concentrated. The crude residue was purified by FCC (5-100% EtOAc in hexanes) to give the title compound (57 mg, 50% yield). LC-MS m/z 210 (M+H⁺).

### Step 7: 6-Amino-7-bromo-3-(difluoromethyl)-3H-imidazo[4,5-b]pyridine-5-carbonitrile

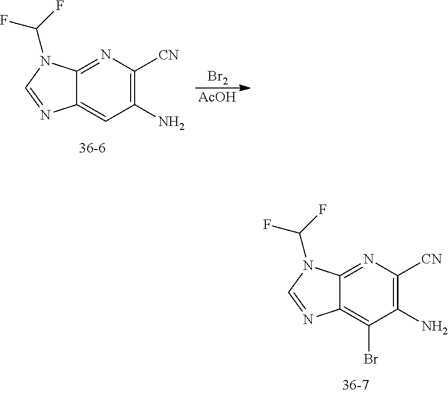

To a suspension of 6-amino-3-(difluoromethyl)imidazo[4,5-b]pyridine-5-carbonitrile (57 mg, 0.27 mmol, 1 equiv.) in glacial acetic acid (2.7 mL) was added bromine (70 μL, 1.37 mmol, 5.0 equiv.), and the mixture was stirred at room temperature for 90 min. The resulting mixture was concentrated to dryness, diluted with sat NaHCO₃ (10 mL) and extracted with EtOAc (2×25 mL). The combined organic layers were dried over MgSO₄, filtered, and concentrated to give the title compound (75 mg, 96% yield), which was sufficiently pure to be used without further purification. LC-MS m/z 288, 290 (M+H⁺).

### Step 8: 6-Amino-3-(difluoromethyl)-7-(3-methoxy-2,6-dimethylphenyl)-3H-imidazo[4,5-b]pyridine-5-carbonitrile

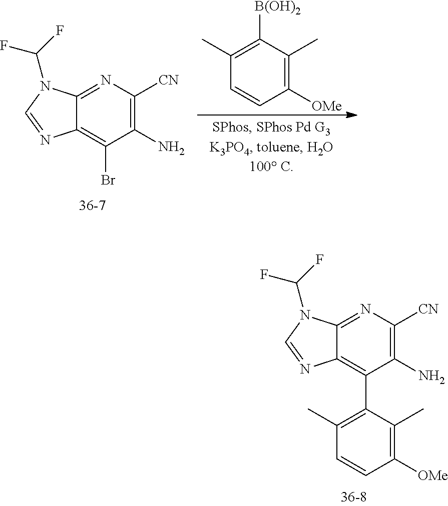 [SMILES: N#Cc1nc2c(ncn2C(F)F)c(Br)c1N ; COc1ccc(C)c(B(O)O)c1C ; COc1ccc(C)c(-c2c(N)c(C#N)nc3c2ncn3C(F)F)c1C]

A screw cap vial was charged with 6-amino-7-bromo-3-(difluoromethyl)imidazo[4,5-b]pyridine-5-carbonitrile (83 mg, 0.288 mmol, 1 equiv.), (3-methoxy-2,6-dimethyl-phenyl)boronic acid (155 mg, 0.86 mmol, 3.0 equiv.). SPhos (35 mg, 0.0853 mmol, 0.3 equiv.), SPhos Pd G3 (67 mg, 0.0859 mmol, 0.3 equiv.) and K₃PO₄ (245 mg, 1.15 mmol, 4.0 equiv.), then evacuated and refilled with nitrogen gas. Toluene sparged with nitrogen gas (3.6 mL) and water (0.6 mL) were added, and the mixture was heated at 100° C. for 2 hr. The resulting mixture was partitioned between EtOAc and water. The aqueous layer was extracted with additional EtOAc, and the combined organic layers were dried over MgSO₄, filtered and concentrated. The residue was adsorbed onto silica gel and subjected to FCC (5-60% EtOAc in hexanes) to give the title compound (50 mg, 51% Yield). LC-MS m/z 344 (M+H⁺).

### Step 9: 6-Amino-3-(difluoromethyl)-7-(3-methoxy-2,6-dimethylphenyl)-3H-imidazo[4,5-b]pyridine-5-carboxamide

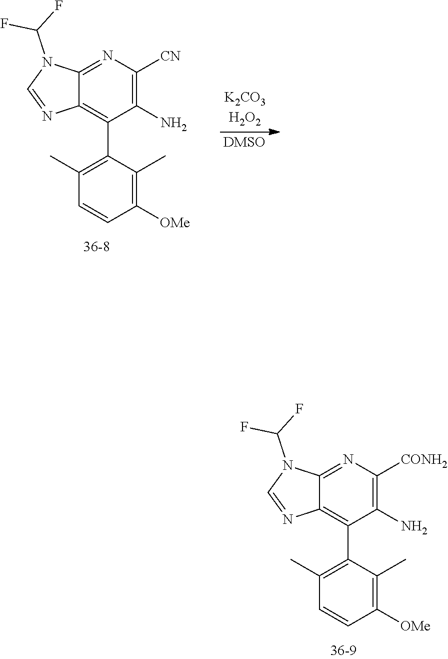 [SMILES: COc1ccc(C)c(-c2c(N)c(C#N)nc3c2ncn3C(F)F)c1C ; COc1ccc(C)c(-c2c(N)c(C(N)=O)nc3c2ncn3C(F)F)c1C]

A mixture of 6-amino-3-(difluoromethyl)-7-(3-methoxy-2,6-dimethyl-phenyl)imidazo[4,5-b]pyridine-5-carbonitrile (50 mg, 0.146 mmol, 1 equiv.), potassium carbonate (100 mg, 0.724 mmol, 5 equiv.), 30% hydrogen peroxide (0.4 mL) and DMSO (2 mL) was stirred at room temperature for 1 hr. The reaction mixture was diluted with water and extracted with EtOAc. The organic layer was washed with water, dried over MgSO₄, filtered, and concentrated to give the title compound (53 mg, 100% yield). LC-MS m/z 362 (M+H⁺).

### Step 10: 6-Amino-3-(difluoromethyl)-7-(3-hydroxy-2,6-dimethylphenyl)-3H-imidazo[4,5-b]pyridine-5-carboxamide

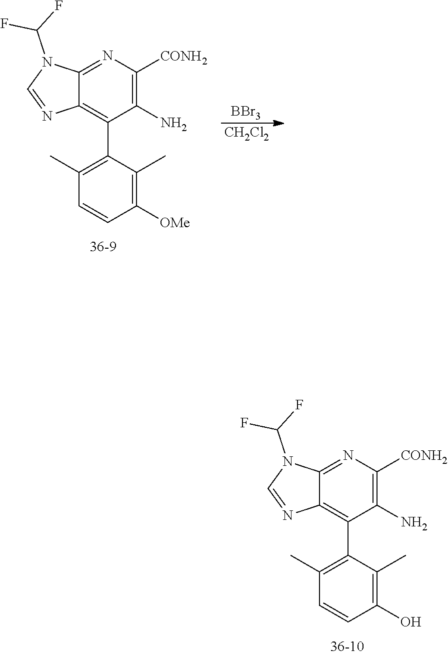

To a solution of 6-amino-3-(difluoromethyl)-7-(3-methoxy-2,6-dimethyl-phenyl)imidazo[4,5-b]pyridine-5-carboxamide (53 mg, 0.147 mmol, 1 equiv.) in anhydrous dichloromethane (7 mL) was added dropwise BBr₃ in CH₂Cl₂ (1 M, 0.6 mL, 0.6 mmol, 4 equiv.). The mixture was stirred at room temperature for 1 hr, then cooled to 0° C., and poured into a pre-cooled, rapidly stirring mixture of sat NaHCO₃ (50 mL) and EtOAc (50 mL) at 0° C. The aqueous layer was extracted with additional EtOAc (25 mL), and the combined organic layers were dried over MgSO₄, filtered, and concentrated to give the title compound (44 mg, 86% yield). LC-MS m/z 348 (M+H⁺).

### Step 11: (P)-6-amino-3-(difluoromethyl)-7-(3-hydroxy-2,6-dimethylphenyl)-3H-imidazo[4,5-b]pyridine-5-carboxamide (72)

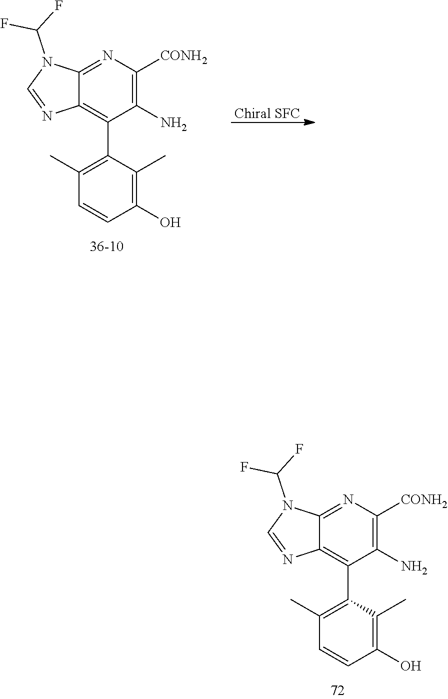

Chiral SFC separation of 6-amino-3-(difluoromethyl)-7-(3-hydroxy-2,6-dimethylphenyl)-3H-imidazo[4,5-b]pyridine-5-carboxamide (44 mg, 0.13 mmol) (Instrument: Waters SFC Prep 150 Mgm; Column: Daicel Chiralpak OD, 30×150 mm, 5 um; Conditions: Isocratic at 30% methanol with 70% CO₂: Flow Rate: 100 mL/min) provided the title compound (72, 14.7 mg). LCMS: m/z 348 [M+H⁺]. ¹H NMR (400 MHz, DMSO) δ 9.27 (s, 1H), 8.79 (s, 1H), 8.25 (s, 1H), 8.15 (t, J=58.9 Hz, 1H), 7.60 (s, 1H), 7.00 (d, J=8.3 Hz, 1H), 6.82 (d, J=8.3 Hz, 1H), 6.05 (s, 2H), 1.77 (s, 3H), 1.70 (s, 3H).

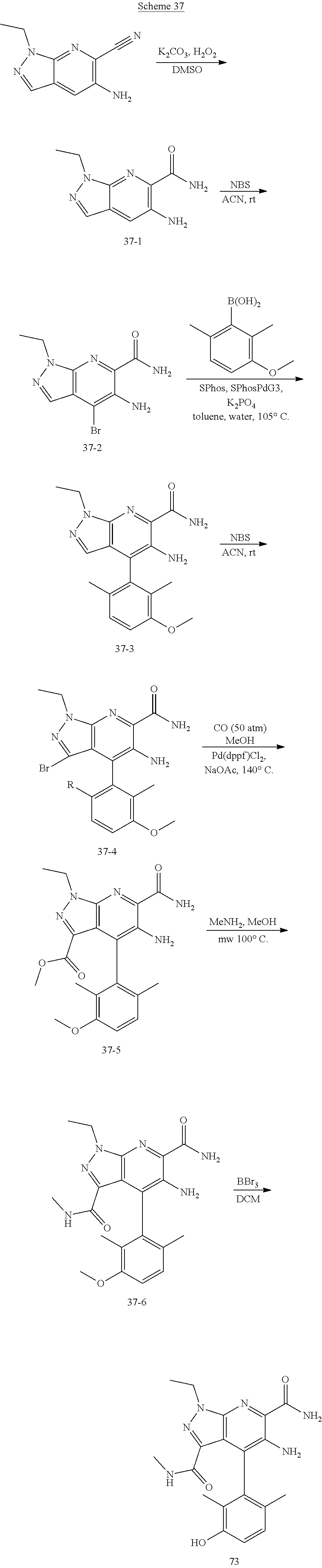

### Example 62. 5-Amino-1-ethyl-4-(3-hydroxy-2,6-dimethylphenyl)-N³-methyl-1H-pyrazolo[3,4-b]pyridine-3,6-dicarboxamide (73)

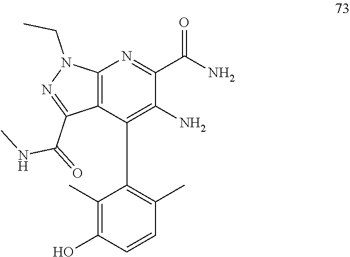 [SMILES: CCn1nc(C(=O)NC)c2c(-c3c(C)ccc(O)c3C)c(N)c(C(N)=O)nc21]

### Step 1: 5-Amino-1-ethyl-1H-pyrazolo[3,4-b]pyridine-6-carboxamide

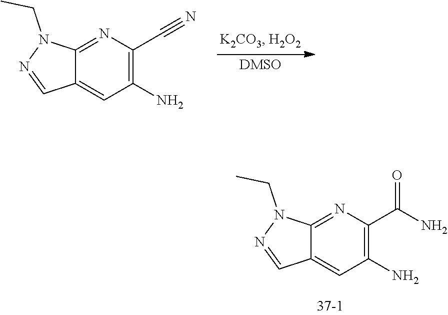

To a solution of 5-amino-1-ethyl-pyrazolo[3,4-b]pyridine-6-carbonitrile (320 mg, 1.71 mmol, 1 equiv.) in DMSO (3 mL) was added potassium carbonate (0.88 g, 6.4 mmol, 3.74 equiv.) followed by dropwise adding hydrogen peroxide in water (30% m/m, 0.80 g, 71 mmol, 42 equiv.), and the reaction was stirred at room temperature until completion. The mixture was quenched water and extracted with EtOAc (3×). The combined organic layers were dried with Na₂SO₄, filtered, and the solvent removed. The residue was purified by silica gel column (0-70% EtOAc in hexanes) to give 5-amino-1-ethyl-pyrazolo[3,4-b]pyridine-6-carboxamide (328 mg, 94%). LCMS m/z 206 (M+H⁺).

### Step 2: 5-Amino-4-bromo-1-ethyl-1H-pyrazolo[3,4-b]pyridine-6-carboxamide

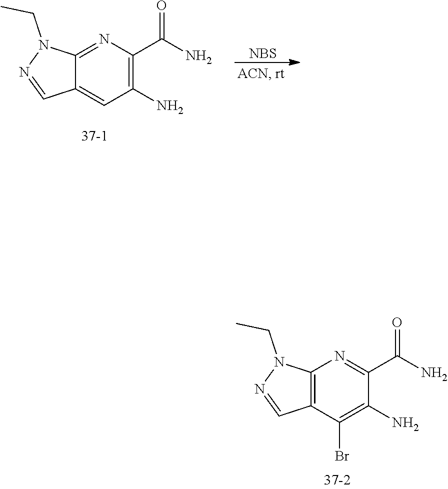

To a solution of 5-amino-1-ethyl-pyrazolo[3,4-b]pyridine-6-carboxamide (0.328 g, 1.60 mmol, 1 equiv.) in MeCN (12 mL) was added solid NBS (285 mg, 1.60 mmol, 1 equiv.) at room temperature. The reaction mixture was stirred for 30 min. The reaction was evaporated to dryness and the residue subjected to silica gel column (0-20% EtOAc in hexanes) to give 5-amino-4-bromo-1-ethyl-pyrazolo[3,4-b]pyridine-6-carboxamide (540 mg, crude) of sufficient purity for the next step. LCMS m/z 284/286 (M+H⁺).

### Step 3: 5-Amino-1-ethyl-4-(3-methoxy-2,6-dimethylphenyl)-1H-pyrazolo[3,4-b]pyridine-6-carboxamide

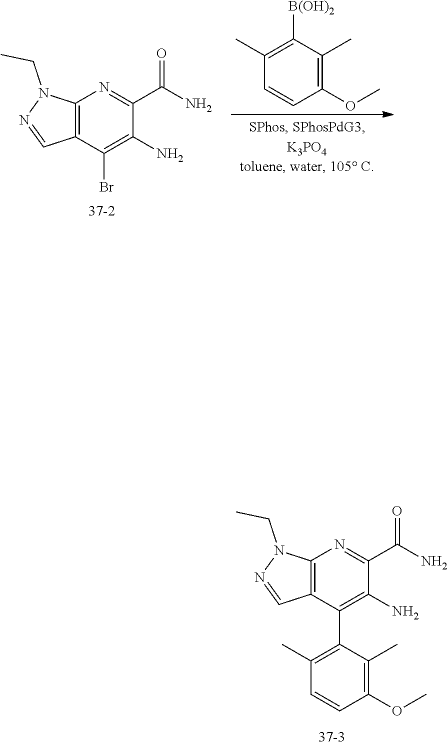

A mixture of 5-amino-4-bromo-1-ethyl-pyrazolo[3,4-b]pyridine-6-carboxamide (540 mg, crude), (3-methoxy-2,6-dimethyl-phenyl)boronic acid (0.600 g, 3.33 mmol), SPhosPdG₃ (0.170 g, 0.218 mmol), SPhos (0.109 g, 0.266 mmol), and potassium phosphate (1.13 g, 5.32 mmol) in toluene (12 mL) and water (3 mL) was stirred at 105° C. in a sealed 40-mL vial for 2 hr. The reaction mixture was diluted with water and extracted with EtOAc (3×). The organic layer was washed with brine, and concentrated. The residue was purified by silica gel column, (0-40% EtOAc in hexanes) to give the product 5-amino-1-ethyl-4-(3-methoxy-2,6-dimethyl-phenyl)pyrazolo[3,4-b]pyridine-6-carboxamide (84 mg, 16%). LCMS m/z 340 (M+H⁺).

### Step 4: 5-Amino-3-bromo-1-ethyl-4-(3-methoxy-2,6-dimethylphenyl)-1H-pyrazolo[3,4-b]pyridine-6-carboxamide

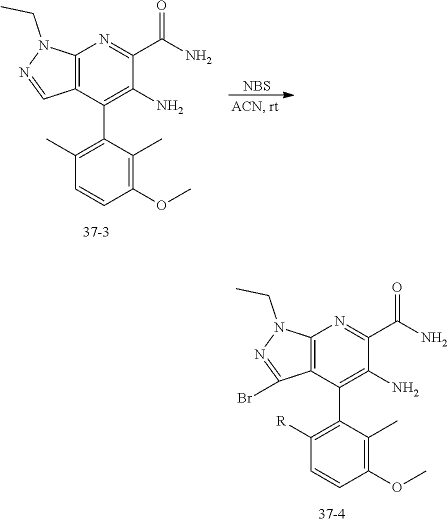

To a solution of 5-amino-1-ethyl-4-(3-methoxy-2,6-dimethyl-phenyl)pyrazolo[3,4-b]pyridine-6-carboxamide (84.0 mg, 0.248 mmol, 1 equiv.) in MeCN (5 mL) at room temperature was added NBS (44.0 mg, 0.247 mmol, 1 equiv.) and the mixture was stirred for 5 min. The reaction mixture was quenched with sat NaHCO₃ (2 mL), concentrated to remove the MeCN, and extracted with DCM. The DCM extract was evaporated and the residue purified via silica gel column (0-40% EtOAc in hexanes) to give 5-amino-3-bromo-1-ethyl-4-(3-methoxy-2,6-dimethyl-phenyl)pyrazolo[3,4-b]pyridine-6-carboxamide (80 mg, 77%). LCMS: 418/420 (M+H⁺).

### Step 5: Methyl 5-amino-6-carbamoyl-1-ethyl-4-(3-methoxy-2,6-dimethylphenyl)-1H-pyrazolo[3,4-b]pyridine-3-carboxylate

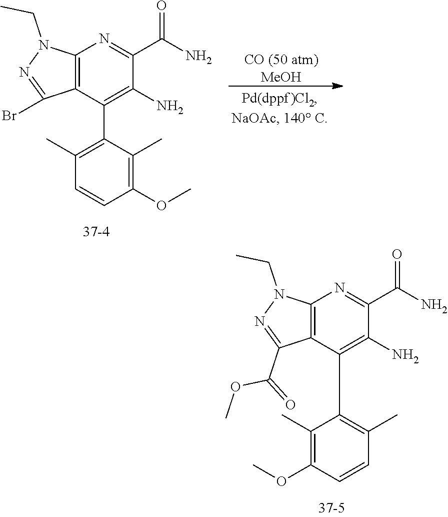 [SMILES: CCn1nc(Br)c2c(-c3c(C)ccc(OC)c3C)c(N)c(C(N)=O)nc21 ; "CO (50 atm)MeOH" ; CCn1nc(C(=O)OC)c2c(-c3c(C)ccc(OC)c3C)c(N)c(C(N)=O)nc21]

A mixture of 5-amino-3-bromo-1-ethyl-4-(3-methoxy-2,6-dimethyl-phenyl)pyrazolo[3,4-b]pyridine-6-carboxamide (80.0 mg, 0.191 mmol, 1 equiv.). Pd(dppf)Cl₂ (16.0 mg, 0.0196 mmol, 0.1 equiv.), sodium acetate (50.0 mg, 0.610 mmol, 3.2 equiv.) in MeOH (15 mL) and DMF (2.0 mL) was stirred under carbon monoxide gas (36 atm at room temperature) and heated at 120° C. for 1 day (pressure at 120° C. was 51 atm). The reaction mixture was recharged CO (32 atm) and heated at 140° C. for 1 day (52 atm at 140° C.). The reaction mixture was concentrated and the residue purified by silica gel column (0-50% EtOAc in hexanes), to give methyl 5-amino-6-carbamoyl-1-ethyl-4-(3-methoxy-2,6-dimethyl-phenyl)pyrazolo[3,4-b]pyridine-3-carboxylate (50 mg, 51%). LCMS m/z 398 (MS+H+).

### Step 6: 5-Amino-1-ethyl-4-(3-methoxy-2,6-dimethylphenyl)-N³-methyl-1H-pyrazolo[3,4-b]pyridine-3,6-dicarboxamide

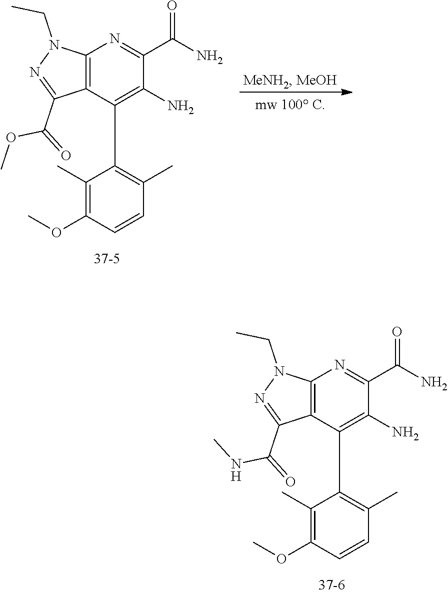

A solution of methyl 5-amino-6-carbamoyl-1-ethyl-4-(3-methoxy-2,6-dimethyl-phenyl)pyrazolo[3,4-b]pyridine-3-carboxylate (50 mg, 0.126 mmol) and 40% MeNH₂ in MeOH (excess) was stirred at 100° C. under microwave condition for 4 hours (pressure 6-8 atm) and concentrated to dryness. The residue was partitioned between EtOAc and NaHCO₃. The aqueous layer was extracted with EtOAc (2×), the organic layers were combined, dried over Na₂SO₄, and evaporated to give 5-amino-1-ethyl-4-(3-methoxy-2,6-dimethyl-phenyl)-N³-methyl-pyrazolo[3,4-b]pyridine-3,6-dicarboxamide (26 mg, 52%) of sufficient purity for the next step. LCMS m/z 397 (M+H+).

### Step 7: 5-Amino-1-ethyl-4-(3-hydroxy-2,6-dimethylphenyl)-N³-methyl-1H-pyrazolo[3,4-b]pyridine-3,6-dicarboxamide. (73)

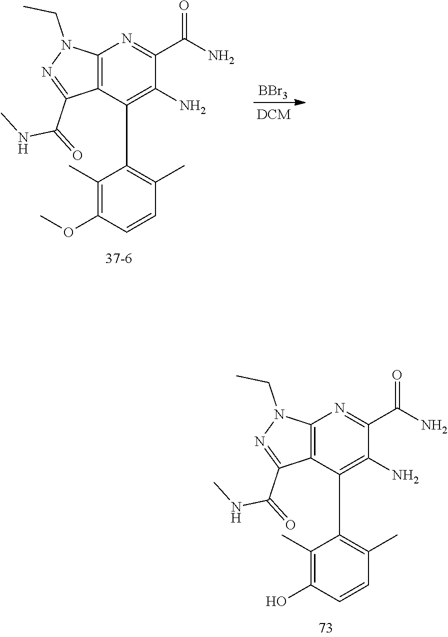

Crude 5-amino-1-ethyl-4-(3-methoxy-2,6-dimethyl-phenyl)-N³-methyl-pyrazolo[3,4-b]pyridine-3,6-dicarboxamide (26 mg) was dissolved in DCM (3 mL). BBr₃ (1 M in DCM, 0.5 mL) was added and the mixture was stirred for 30 min. A second portion of BBr₃ (1M in DCM, 0.5 mL) was added and the mixture was stirred at room temperature for 1 hr. The reaction was quenched with aqueous NaHCO₃, extracted with EtOAc (3×), and the combined organic layers were concentrated to dryness. The residue was subjected to hydrogenation with Pd on carbon (10%, 10 mg) in MeOH (20 mL) under 1 atm of hydrogen gas at room temperature overnight. The reaction mixture was filtered through Celite®, concentrated, and the residue was purified by prep-HPLC (10-50% ACN in water, 0.1% FA), giving 5-amino-1-ethyl-4-(3-hydroxy-2,6-dimethyl-phenyl)-N3-methyl-pyrazolo[3,4-b]pyridine-3,6-dicarboxamide (7.0 mg, 28%). ¹H NMR (400 MHz, DMSO) δ 9.04 (s, 1H), 8.45 (d, J=2.7 Hz, 1H), 7.68 (d, J=2.7 Hz, 1H), 7.63 (q, J=4.6 Hz, 1H), 6.85 (d, J=8.2 Hz, 1H), 6.67 (d, J=8.2 Hz, 1H), 5.65 (s, 2H), 4.50 (q, J=7.2 Hz, 2H), 2.37 (d, J=4.7 Hz, 3H), 1.64 (s, 3H), 1.59 (s, 3H), 1.41 (t, J=7.2 Hz, 3H). LCMS m/z 383 (M+H+).

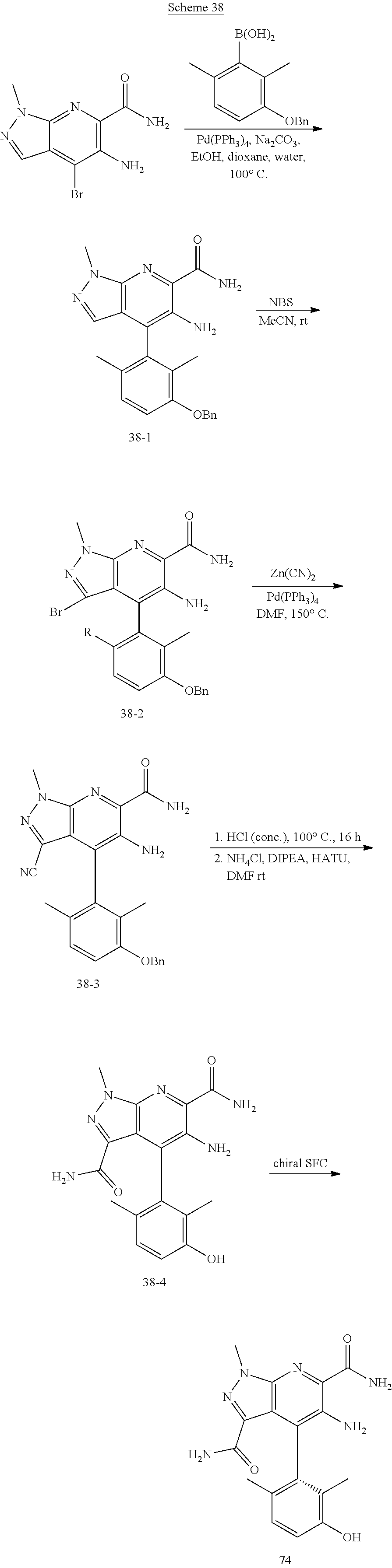

### Example 63. (M)-5-Amino-4-(3-hydroxy-2,6-dimethylphenyl)-1-methyl-1H-pyrazolo[3,4-b]pyridine-3,6-dicarboxamide (74)

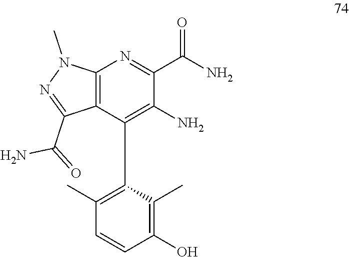

### Step 1: 5-Amino-4-(3-(benzyloxy)-2,6-dimethylphenyl)-1-methyl-1H-pyrazolo[3,4-b]pyridine-6-carboxamide

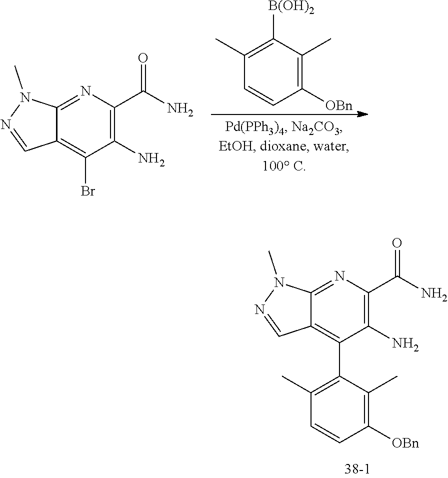 [SMILES: Cn1ncc2c(Br)c(N)c(C(N)=O)nc21 ; Cc1ccc(OCc2ccccc2)c(C)c1B(O)O ; Cc1ccc(OCc2ccccc2)c(C)c1-c1c(N)c(C(N)=O)nc2c1cnn2C]

A solution of 5-amino-4-bromo-1-methyl-pyrazolo[3,4-b]pyridine-6-carboxamide (0.102 g, 0.378 mmol, 1 equiv.), (3-benzyloxy-2,6-dimethyl-phenyl)boronic acid (0.300 g, 1.17 mmol, 3.1 equiv.), tetrakis(triphenylphosphine)palladium(0) (150 mg, 0.130 mmol, 0.34 equiv.), sodium carbonate (250 mg, 2.36 mmol, 6.2 equiv.) in EtOH (2 mL), dioxane (2 mL), and water (0.8 mL) was stirred at 100° C. in a sealed tube for 2 days. The mixture was partitioned between EtOAc and water, the aqueous layer extracted with EtOAc (3×). The combined organic layers were concentrated and the residue was purified by silica gel column (0-40% EtOAc in hexanes) to give 5-amino-4-(3-benzyloxy-2,6-dimethyl-phenyl)-1-methyl-pyrazolo[3,4-b]pyridine-6-carboxamide (92 mg, 61%), LCMS m/z 402 (M+H+).

### Step 2: 5-Amino-4-(3-(benzyloxy)-2,6-dimethylphenyl)-3-bromo-1-methyl-1H-pyrazolo[3,4-b]pyridine-6-carboxamide

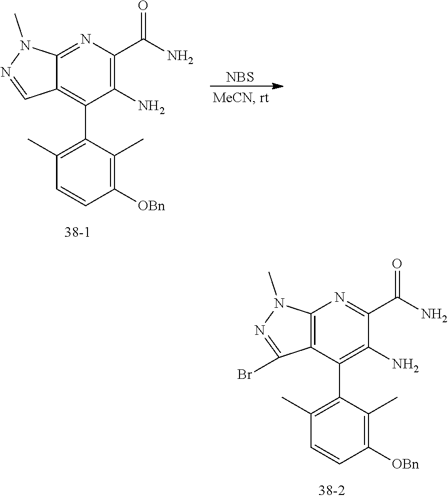

To a mixture of 5-amino-4-(3-benzyloxy-2,6-dimethyl-phenyl)-1-methyl-pyrazolo[3,4-b]pyridine-6-carboxamide (92.0 mg, 0.229 mmol, 1 equiv.) in MeCN (5 mL) was added NBS (40.7 mg, 0.229 mmol, 1 equiv.). The reaction mixture was stirred at room temperature for 1 hr. concentrated to dryness, and purified by silica gel column chromatography (0-50% EtOAc in hexanes) to give 5-amino-4-(3-benzyloxy-2,6-dimethyl-phenyl)-3-bromo-1-methyl-pyrazolo[3,4-b]pyridine-6-carboxamide (82.0 mg, 74% Yield). LCMS m/z 480/482 (M+H+).

### Step 3: 5-Amino-4-(3-(benzyloxy)-2,6-dimethylphenyl)-3-cyano-1-ethyl-1H-pyrazolo[3,4-b]pyridine-6-carboxamide

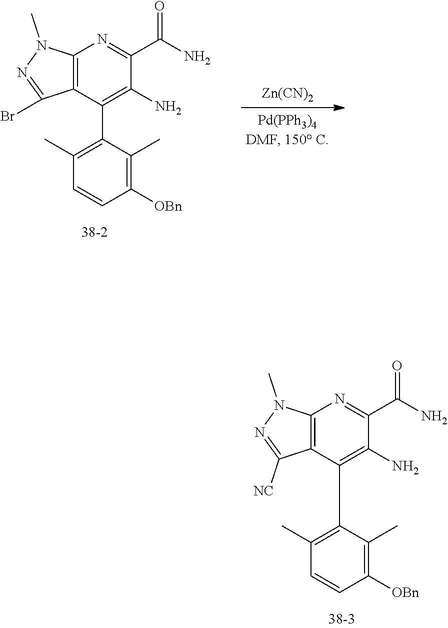 [SMILES: Cc1ccc(OCc2ccccc2)c(C)c1-c1c(N)c(C(N)=O)nc2c1c(Br)nn2C ; "Zn(CN)2" ; Cc1ccc(OCc2ccccc2)c(C)c1-c1c(N)c(C(N)=O)nc2c1c(C#N)nn2C]

A mixture of 5-amino-4-(3-benzyloxy-2,6-dimethyl-phenyl)-3-bromo-1-methyl-pyrazolo[3,4-b]pyridine-6-carboxamide (82.0 mg, 0.171 mmol, 1 equiv.), dicyanozinc (70.0 mg, 0.596 mmol, 3.5 equiv.), Pd(PPh₃)₄ (30.0 mg, 0.0260 mmol, 0.15 equiv.) in DMF (2 mL) was stirred under nitrogen at 150° C. for 16 hr and cooled to room temperature. The reaction mixture was poured into water (20 mL), the resulting suspension was filtered, rinsed with water and dried to give 5-amino-4-(3-benzyloxy-2,6-dimethyl-phenyl)-3-cyano-1-methyl-pyrazolo[3,4-b]pyridine-6-carboxamide (62.0 mg, 85%) of sufficient purity for the next step. LCMS m/z 427 (M+H+).

### Step 4: 5-Amino-4-(3-hydroxy-2,6-dimethylphenyl)-1-methyl-1H-pyrazolo[3,4-b]pyridine-3,6-dicarboxamide. (74)

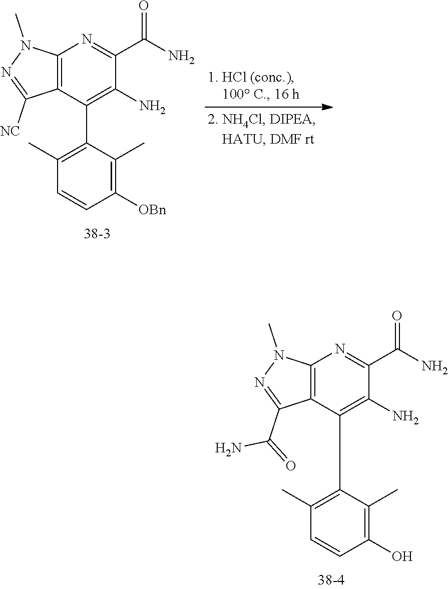 [SMILES: Cc1ccc(OCc2ccccc2)c(C)c1-c1c(N)c(C(N)=O)nc2c1c(C#N)nn2C ; Cc1ccc(O)c(C)c1-c1c(N)c(C(N)=O)nc2c1c(C(N)=O)nn2C]

Crude 5-amino-4-(3-benzyloxy-2,6-dimethyl-phenyl)-3-cyano-1-methyl-pyrazolo[3,4-b]pyridine-6-carboxamide (62.0 mg, 0.145 mmol) in HCl (conc, 6 mL) was stirred at 100° C. overnight and cooled to room temperature. The reaction mixture was adjusted to pH ~4 with aqueous NaOH (4M) and extracted with EtOAc (3×). The combined organic layers were evaporated. The residue was combined with NH₄Cl (100 mg), and DIPEA (0.6 mL) in DMF (3 mL) was added HATU (200 mg), and the reaction mixture was stirred at room temperature for 1 hr. The reaction mixture was partitioned between EtOAc and water and the aqueous layer was extracted with EtOAc (3×). The combined organic layers were washed with brine, dried over Na₂SO₄, and evaporated. The residue was purified by silica gel column (0-8% MeOH in DCM). The solid was washed with MeOH (2 mg) to give 28 mg of the title compound (28.0 mg, 54%). ¹H NMR (400 MHz, DMSO) δ 9.07 (s, 1H), 8.54 (s, 1H), 7.77 (s, 1H), 7.19 (s, 1H), 6.91 (d, J=7.9 Hz, 2H), 6.72 (d, J=8.1 Hz, 1H), 5.72 (s, 2H), 4.13 (s, 3H), 1.72 (s, 3H), 1.66 (s, 3H). LCMS m/z 355 (M+H+).

### Step 5: (M)-5-Amino-4-(3-hydroxy-2,6-dimethylphenyl)-1-methyl-1H-pyrazolo[3,4-b]pyridine-3,6-dicarboxamide. (74)

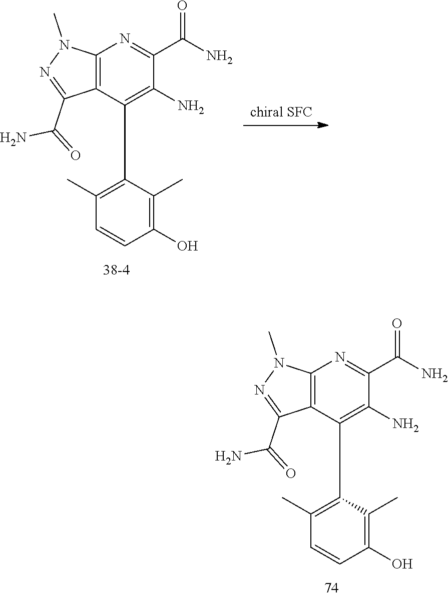

Chiral SFC separation of 5-Amino-4-(3-hydroxy-2,6-dimethylphenyl)-1-methyl-1H-pyrazolo[3,4-b]pyridine-3,6-dicarboxamide (25 mg, 0.070 mmol) (Instrument: Waters SFC Prep 150 Mgm; Column: Daicel Chiralpak OD, 30×150 mm, 5 um; Conditions: Isocratic at 35% methanol with 65% CO₂; Flow Rate: 100 mL/min) provided the title compound (74, 11 mg). LCMS: m/z 355 [M+H⁺]. ¹H NMR (400 MHz, DMSO) δ 9.07 (s, 1H), 8.54 (d, J=2.7 Hz, 1H), 7.77 (d, J=2.8 Hz, 1H), 7.19 (s, 1H), 6.94-6.87 (m, 2H), 6.73 (d, J=8.2 Hz, 1H), 5.72 (s, 2H), 4.13 (s, 3H), 1.72 (s, 3H), 1.66 (s, 3H).

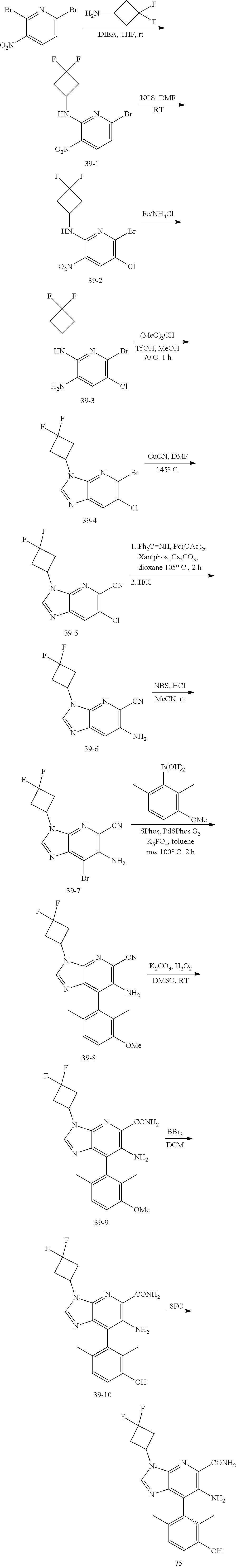

### Example 64. (P)-6-amino-3-(3,3-difluorocyclobutyl)-7-(3-hydroxy-2,6-dimethylphenyl)-3H-imidazo[4,5-b]pyridine-5-carboxamide (75)

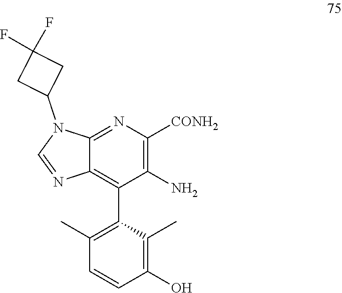

### Step 1: 6-Bromo-N-(3,3-difluorocyclobutyl)-3-nitropyridin-2-amine

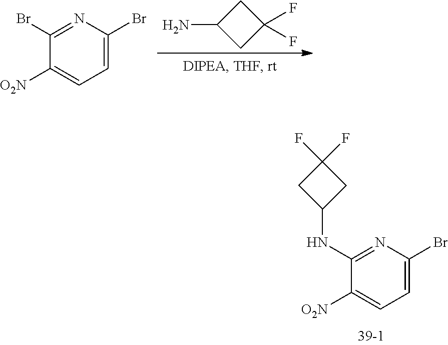 [SMILES: O=[N+]([O-])c1ccc(Br)nc1Br ; NC1CC(F)(F)C1 ; O=[N+]([O-])c1ccc(Br)nc1NC1CC(F)(F)C1]

To a mixture of 2,6-dibromo-3-nitro-pyridine (42.3 g, 150 mmol, 1 equiv.) and DIPEA (1 equiv.) in THF (500 mL) at room temperature was added solid 3,3-difluorocyclobutanamine (16.2 g, 151 mmol, 1 equiv.). After addition, the reaction mixture was stirred at room temperature for 2 h. The resulting suspension was filtered and the solid was washed with MTBE. The filtrate and washing were combined and concentrated to dryness to give the title compound (47.8 g, 100%) of sufficient purity for the next step. LCMS m/z 308/310 (M+H+).

### Step 2: 6-Bromo-5-chloro-N-(3,3-difluorocyclobutyl)-3-nitropyridin-2-amine

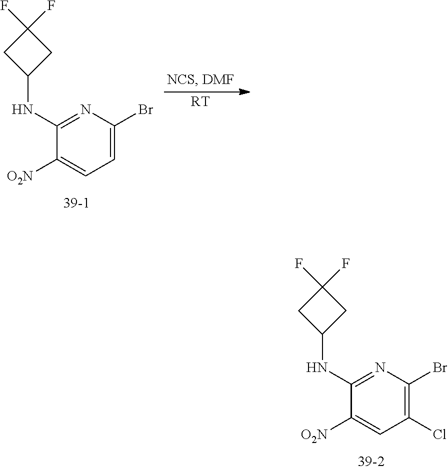

To a solution of crude 6-bromo-N-(3,3-difluorocyclobutyl)-3-nitropyridin-2-amine (47.8 g, 150 mmol, 1 equiv.) in DMF (300 mL) was added NCS (24.0, 180 mmol, 1.2 equiv.) at room temperature and the mixture was stirred overnight. Water was added, the mixture was filtered, washed with water, and dried to give 6-bromo-5-chloro-N-(323-difluorocyclobutyl)-3-nitro-pyridin-2-amine (48.3 g, 94%). LCMS m/z 342/344 (M+H+).

### Step 3: 6-Bromo-5-chloro-N²-(3,3-difluorocyclobutyl)pyridine-2,3-diamine

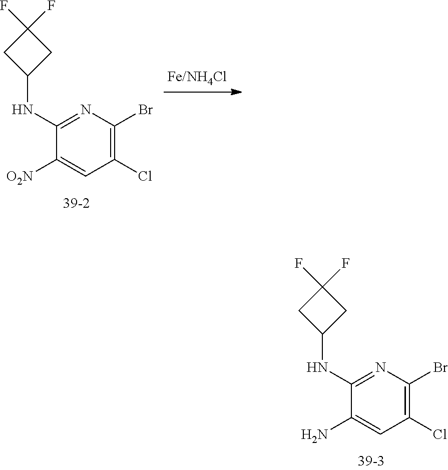 [SMILES: O=[N+]([O-])c1cc(Cl)c(Br)nc1NC1CC(F)(F)C1 ; Nc1cc(Cl)c(Br)nc1NC1CC(F)(F)C1]

A mixture of 6-bromo-5-chloro-N-(3,3-difluorocyclobutyl)-3-nitro-pyridin-2-amine (27.0 g, 78.8 mmol, 1 equiv.), iron powder (5 equiv.), and NH₄Cl (10 equiv.) in EtOH (200 mL) and water (30 mL) was stirred at 70° C. for 1 hr. The mixture was filtered through Celite® and washed with MeOH (3×50 mL). The combined filtrates were evaporated to dryness and the residue was triturated with water. The mixture was filtered and dried to give 6-bromo-5-chloro-N2-(3,3-difluorocyclobutyl)pyridine-2,3-diamine (24.1 g, 98%) of sufficient purity for the next step. LCMS m/z 314 (M+H+).

### Step 4: 5-Bromo-6-chloro-3-(3,3-difluorocyclobutyl)-3H-imidazo[4,5-b]pyridine

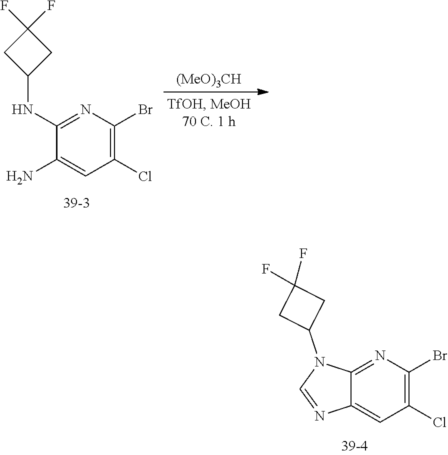 [SMILES: "(MeO)3CH" ; Nc1cc(Cl)c(Br)nc1NC1CC(F)(F)C1 ; FC1(F)CC(n2cnc3cc(Cl)c(Br)nc32)C1]

A mixture of 6-bromo-5-chloro-N²-(3,3-difluorocyclobutyl)pyridine-2,3-diamine (18.6 g, 59.5 mmol, 1 equiv.), trimethoxymethane (18.0 g, 170 mmol, 2.9 equiv.), and sulfamic acid (0.550 g, 5.66 mmol, 0.1 equiv.) in MeOH was stirred at room temperature overnight. The mixture was concentrated, the residue was treated with aqueous NaHCO₃ (2 M, 300 mL), and resulting suspension was stirred at room temperature for 1 hr. The mixture was filtered, washed with water then DCM to give crude 5-bromo-6-chloro-3-(3,3-difluorocyclobutyl)imidazo[4,5-b]pyridine (15.2 g, 79%). LCMS m/z 324 (M+H+).

### Step 5: 6-Chloro-3-(3,3-difluorocyclobutyl)-3H-imidazo[4,5-b]pyridine-5-carbonitrile

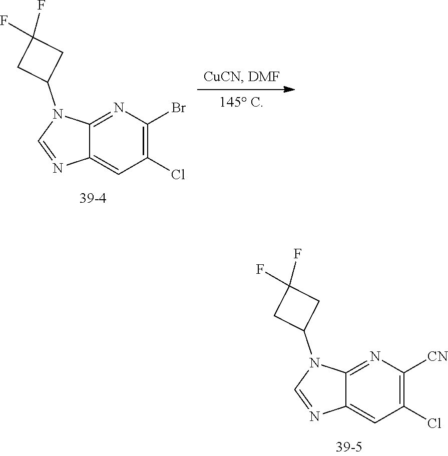 [SMILES: FC1(F)CC(n2cnc3cc(Cl)c(Br)nc32)C1 ; N#Cc1nc2c(cc1Cl)ncn2C1CC(F)(F)C1]

A mixture of 5-bromo-6-chloro-3-(3,3-difluorocyclobutyl)imidazo[4,5-b]pyridine (6.80 g, 21.1 mmol, 1 equiv.) and CuCN (2.20 g, 24.6 mmol, 1.17 equiv.) in DMF (30 mL) was stirred at 145° C. for 16 hr. The mixture was poured onto ice-water (150 mL), filtered, washed with water, and dried to give crude product. The solid was suspended in EtOAc:MeOH (4:1), filtered, and the solvent removed to give 6-chloro-3-(3,3-difluorocyclobutyl)imidazo[4,5-b]pyridine-5-carbonitrile (4.44 g, 78%). LCMS m/z 269 (M+H+).

### Step 6: 6-Amino-3-(3,3-difluorocyclobutyl)-3H-imidazo[4,5-b]pyridine-5-carbonitrile

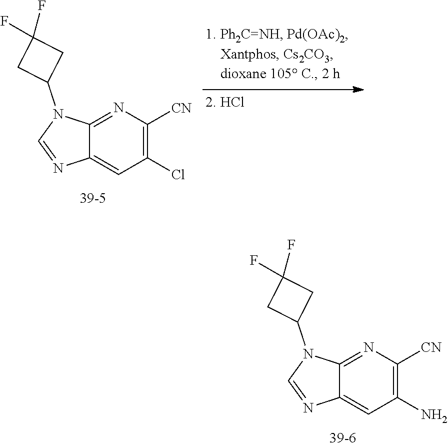 [SMILES: N#Cc1nc2c(cc1Cl)ncn2C1CC(F)(F)C1 ; N#Cc1nc2c(cc1N)ncn2C1CC(F)(F)C1]

6-Chloro-3-(3,3-difluorocyclobutyl)imidazo[4,5-b]pyridine-5-carbonitrile (4.40 g, 16.4 mmol, 1 equiv.), Xantphos (2.00 g, 3.46 mmol, 0.21 equiv.), cesium carbonate (12.0 g, 36.8 mmol, 2.24 equiv.), Pd(OAc)₂ (0.550 g, 2.45 mmol, 0.15 equiv.) in dioxane (60 mL) was stirred at 105° C. for 2 hr. The reaction mixture was diluted with EtOAc (60 mL), filtered through Celite®, and rinsed with EtOAc (3×40 mL). To the filtrate was added concentrated HCl (20 mL) and the resulting mixture was stirred at room temperature for 1 hr. The resulting mixture was neutralized with NaHCO₃ to pH 8-9 and extracted with EtOAc (3×). The combined organic layers were washed with brine, dried over Na₂SO₄, filtered, and concentrated to dryness. The residue was washed with DCM (15 mL) to give crude 6-amino-3-(3,3-difluorocyclobutyl)imidazo[4,5-b]pyridine-5-carbonitrile (3.12 g, 76%). LCMS m/z 250 (M+H+).

### Step 7: 6-Amino-7-bromo-3-(3,3-difluorocyclobutyl)-3H-imidazo[4,5-b]pyridine-5-carbonitrile

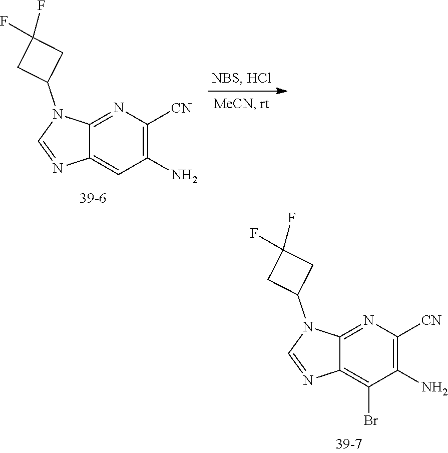 [SMILES: N#Cc1nc2c(cc1N)ncn2C1CC(F)(F)C1 ; N#Cc1nc2c(ncn2C2CC(F)(F)C2)c(Br)c1N]

A suspension of 6-amino-3-(3,3-difluorocyclobutyl)imidazo[4,5-b]pyridine-5-carbonitrile (1.60 g, 6.42 mmol, 1 equiv.) in MeCN (25 mL) was cooled to 0-5° C. HCl (4 M in dioxane, 1.60 mL, 6.40 mmol, 1 equiv.) followed by NBS (1.70 g, 9.55 mmol, 1.49 equiv.). The mixture was stirred at 10° C. for 30 min and the solvent removed. The residue was treated with a sat. aqueous solution of NaHCO₃ and extracted with EtOAc (3×). The combined organic layers were washed with brine, concentrated, and the residue was subjected to silica gel column chromatography to provide 6-amino-7-bromo-3-(3,3-difluorocyclobutyl)imidazo[4,5-b]pyridine-5-carbonitrile (1.51 g, 72%). LCMS m/z 328/330.

### Step 8: 6-Amino-3-(3,3-difluorocyclobutyl)-7-(3-methoxy-2,6-dimethylphenyl)-3H-imidazo[4,5-b]pyridine-5-carbonitrile

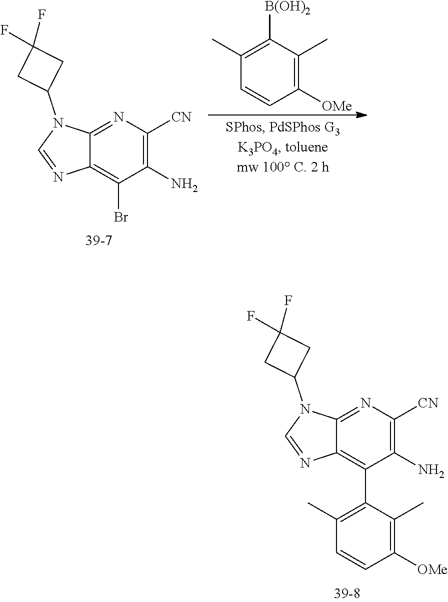 [SMILES: N#Cc1nc2c(ncn2C2CC(F)(F)C2)c(Br)c1N ; COc1ccc(C)c(B(O)O)c1C ; COc1ccc(C)c(-c2c(N)c(C#N)nc3c2ncn3C2CC(F)(F)C2)c1C]

A solution of 6-amino-3-(3,3-difluorocyclobutyl)imidazo[4,5-b]pyridine-5-carbonitrile (1.10 g, 3.35 mmol, 1 equiv.), (3-methoxy-2,6-dimethyl-phenyl)boronic acid (1.44 g, 8.00 mmol, 2.4 equiv.), PdSPhosG3 (350 mg, 0.449 mmol, 0.134 equiv.), SPhos (300 mg, 0.731 mmol, 0.218 equiv.), and K₃PO₄ (2.50 g, 11.8 mmol, 3.52 equiv.) in toluene (25 mL) was stirred at 100° C. for 2 hr. The reaction mixture was cooled to room temperature, diluted with EtOAc. and filtered through Celite®. The filtrate was concentrated and the residue was purified by silica gel chromatography (0-40% EtOAc in hexanes) to afford 6-amino-3-(3,3-difluorocyclobutyl)-7-(3-methoxy-2,6-dimethyl-phenyl)imidazo[4,5-b]pyridine-5-carbonitrile (440 mg, 34%). LCMS m/z 384 (M+H+).

### Step 9: 6-Amino-3-(3,3-difluorocyclobutyl)-7-(3-methoxy-2,6-dimethylphenyl)-3H-imidazo[4,5-b]pyridine-5-carboxamide

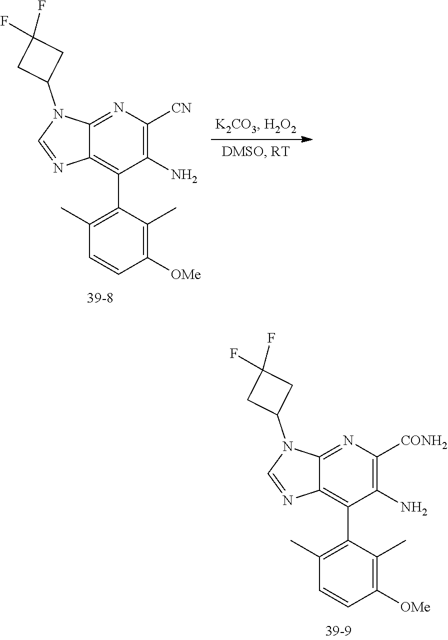 [SMILES: COc1ccc(C)c(-c2c(N)c(C#N)nc3c2ncn3C2CC(F)(F)C2)c1C ; COc1ccc(C)c(-c2c(N)c(C(N)=O)nc3c2ncn3C2CC(F)(F)C2)c1C]

To a solution of 6-amino-3-(3,3-difluorocyclobutyl)-7-(3-methoxy-2,6-dimethyl-phenyl)imidazo[4,5-b]pyridine-5-carbonitrile (440 mg, 1.15 mmol, 1 equiv.) and K₂CO₃ (1.38 g, 10 mmol, 8.7 equiv.) in DMSO (10 mL) was added H₂O₂ (30% in water, 3.0 mL) and the mixture was stirred at room temperature for 30 min. The reaction mixture was diluted with water and filtered. The solid was washed with water and dried to give 6-amino-3-(3,3-difluorocyclobutyl)-7-(3-methoxy-2,6-dimethyl-phenyl)imidazo[4,5-b]pyridine-5-carboxamide (372 mg, 81%) of sufficient purity for the next step. LCMS m/z 402 (M+H+).

### Step 10: 6-Amino-3-(3,3-difluorocyclobutyl)-7-(3-hydroxy-2,6-dimethylphenyl)-3H-imidazo[4,5-b]pyridine-5-carboxamide

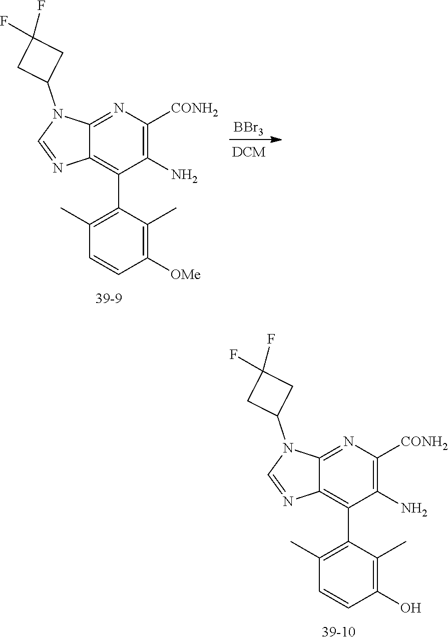

A suspension of crude 6-amino-3-(3,3-difluorocyclobutyl)-7-(3-methoxy-2,6-dimethyl-phenyl)imidazo[4,5-b]pyridine-5-carboxamide (372 mg, 0.927 mmol) in DCM (40 mL) was added BBr₃ (1 M in DCM, 8.0 mL) and the mixture was stirred at room temperature for 1 hr. The reaction was quenched with cold aqueous sat. solution of NaHCO₃, adjusted to pH 9, and the DCM was removed in vacuo. The resulting suspension was filtered, the solid washed with water, and dried to give crude product (330 mg). This material was purified by HPLC (10 to 45% ACN in water with 0.1% FA) to give 6-amino-3-(3,3-difluorocyclobutyl)-7-(3-hydroxy-2,6-dimethyl-phenyl)imidazo[4,5-b]pyridine-5-carboxamide (240 mg, 67%). ¹H NMR (400 MHz, DMSO) δ 9.25 (s, 1H), 8.52 (s, 1H), 8.13 (d, J=3.2 Hz, 1H), 7.55 (d, J=3.3 Hz, 1H), 6.99 (d, J=8.2 Hz, 1H), 6.81 (d, J=8.1 Hz, 1H), 5.87 (s, 2H), 5.16 (td, J=8.2, 4.5 Hz, 1H), 3.49 (dd, J=20.3, 12.1 Hz, 2H), 3.30-3.23 (m, 2H), 1.76 (s, 3H), 1.70 (s, 3H). LCMS m/z 388 (M+H+).

### Step 11: (P)-6-amino-3-(3,3-difluorocyclobutyl)-7-(3-hydroxy-2,6-dimethylphenyl)-3H-imidazo[4,5-b]pyridine-5-carboxamide. (75)

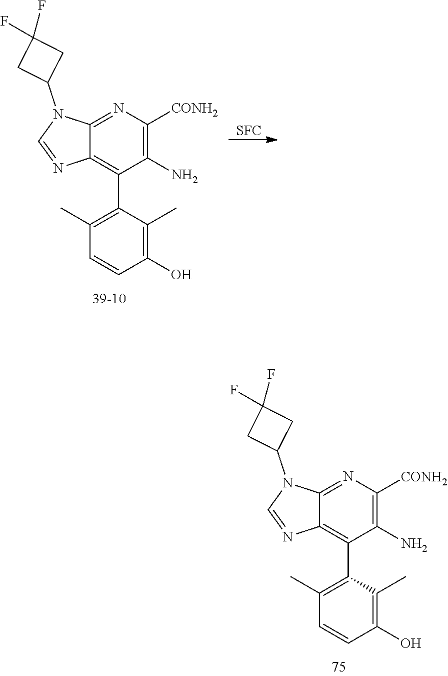

6-Amino-3-(3,3-difluorocyclobutyl)-7-(3-hydroxy-2,6-dimethyl-phenyl)imidazo[4,5-b]pyridine-5-carboxamide (240 mg) was submitted for SFC chiral separation: (Instrument: Waters SFC Prep 150 Mgm; Column: Chiralcel OJ, 30×150 mm, 5 um; Conditions: Isocratic at 25% methanol with 75% CO₂; Flow Rate: 100 mL/min) provided the title compound (75, LCMS: m/z 388 [M+H⁺]. ¹H NMR (400 MHz, DMSO) δ 9.24 (s, 1H), 8.52 (s, 1H), 8.13 (d, J=3.1 Hz, 1H), 7.55 (d, J=3.2 Hz, 1H), 6.99 (d, J=8.2 Hz, 1H), 6.81 (d, J=8.1 Hz, 1H), 5.88 (s, 2H), 5.16 (td, J=8.3, 4.6 Hz, 1H), 3.48 (s, 2H), 3.29 (s, 2H), 1.76 (s, 3H), 1.70 (s, 3H). ¹⁹F NMR (376 MHz, DMSO) δ −82.42 (d, J=196.3 Hz). −98.12 (d, J=196.3 Hz).

### Example 65. (M)-5-Amino-1-ethyl-4-(3-hydroxy-2,6-dimethylphenyl)-1H-pyrrolo[2,3-b]pyridine-3,6-dicarboxamide (76)

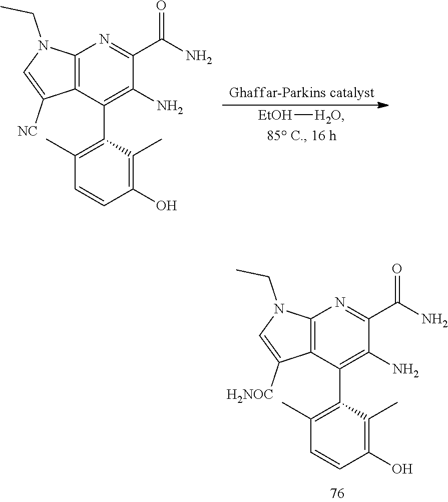

To a solution of (R)-5-amino-3-cyano-1-ethyl-4-(3-hydroxy-2,6-dimethylphenyl)-1H-pyrrolo[2,3-b]pyridine-6-carboxamide (0.177 g, 0.507 mmol, 1 equiv.) in EtOH (10 mL) and water (5 mL), was added Ghaffar-Parkins catalyst (48.0 mg, 0.112 mmol, 0.22 equiv.). The mixture was stirred at 85° C. in a sealed vial overnight. The solution was concentrated to dryness and the solid was purified by silica gel column (0-10% MeOH in DCM) to give the title compound (141 mg, 76%). ¹H NMR (400 MHz, DMSO) δ 9.40 (s, 1H), 8.26 (d, J=3.1 Hz, 1H), 8.21 (s, 1H), 7.49 (d, J=3.1 Hz, 1H), 7.04 (d, J=8.2 Hz, 1H), 6.84 (d, J=8.2 Hz, 1H), 6.59 (s, 1H), 5.68 (s, 2H), 5.08 (s, 1H), 4.36 (q, J=7.2 Hz, 2H), 1.79 (s, 3H), 1.72 (s, 3H), 1.43 (t, J=7.2 Hz, 3H). LCMS: m/z 368 (M+H)⁺.

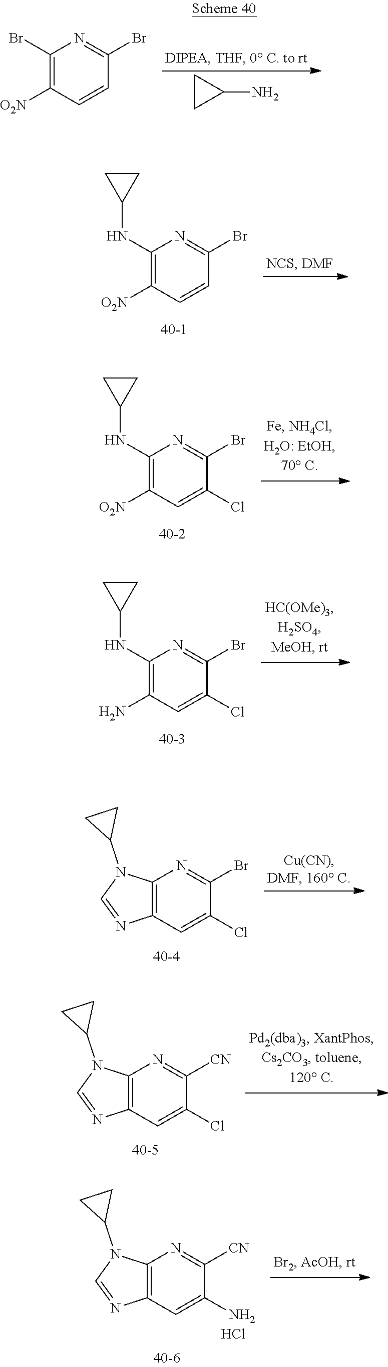

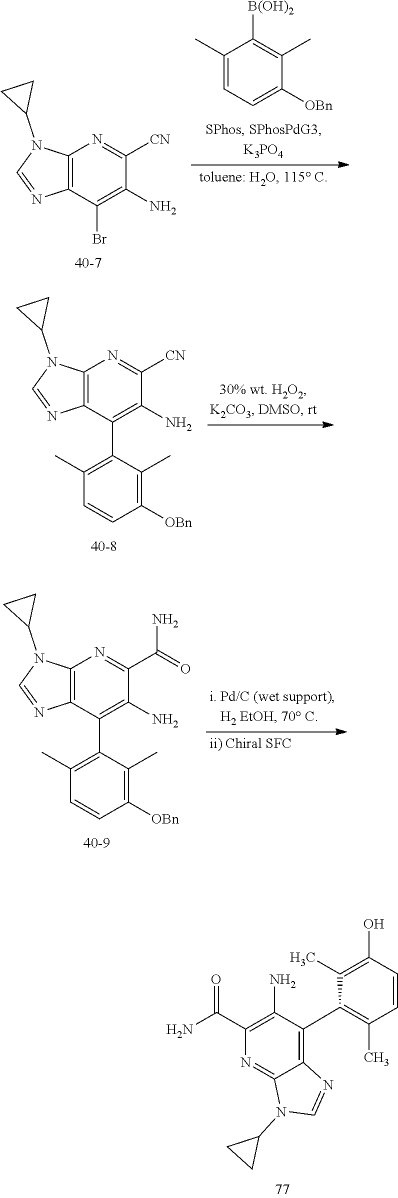

### Example 66: (P)-6-amino-3-cyclopropyl-7-(3-hydroxy-2,6-dimethylphenyl)-3H-imidazo[4,5-b]pyridine-5-carboxamide (77)

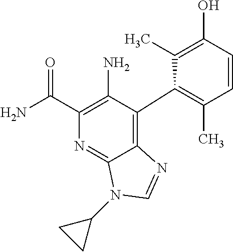

### Step 1: 6-Bromo-N-cyclopropyl-3-nitropyridin-2-amine

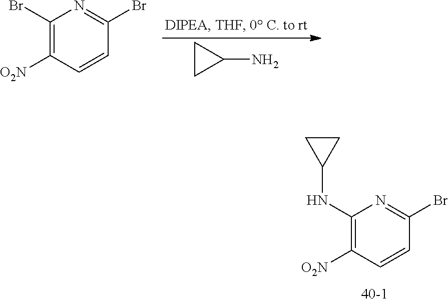

Cyclopropylamine (4.1 mL, 60 mmol, 1 equiv.) was added to a stirring mixture of 2,6-dibromo-3-nitropyridine (17 g, 60 mmol, 1 equiv.) and DIPEA (10 mL, 60 mmol, 1 equiv.) in THF (200 mL) at 0° C. and the reaction mixture was stirred at room temperature overnight. The reaction mixture was filtered and the cake was washed with methyl tert-butyl ether. The filtrate was concentrated to afford the title compound (16 g. crude), which was used without further purification. LC-MS m/z 258/260 (M+H⁺).

### Step 2: 6-Bromo-5-chloro-N-cyclopropyl-3-nitropyridin-2-amine

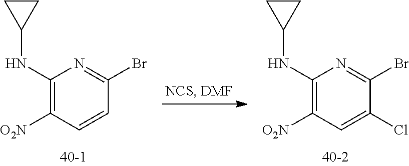 [SMILES: O=[N+]([O-])c1ccc(Br)nc1NC1CC1 ; O=[N+]([O-])c1cc(Cl)c(Br)nc1NC1CC1]

N-Chlorosuccinimide (9.1 g, 68 mmol, 1.15 equiv.) was added to a stirring solution of 6-bromo-N-cyclopropyl-3-nitropyridin-2-amine (15 g, 59 mmol, 1 equiv.) in DMF (80 mL) and the mixture was stirred at room temperature overnight. Water was added to the reaction mixture and the slurry was filtered. The filter cake was washed with water and dried to afford the title compound (18 g) of sufficient purity for the next step. LCMS m/z 292/294 (M+H⁺).

### Step 3: 6-Bromo-5-chloro-N²-cyclopropylpyridine-2,3-diamine

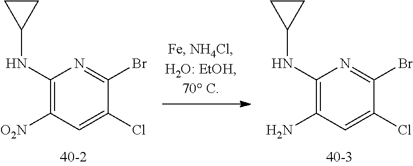 [SMILES: O=[N+]([O-])c1cc(Cl)c(Br)nc1NC1CC1 ; Nc1cc(Cl)c(Br)nc1NC1CC1]

A mixture of crude 6-bromo-5-chloro-N-cyclopropyl-3-nitropyridin-2-amine (14.0 g, 48 mmol, 1 equiv.), iron powder (12.0 g, 215 mmol, 4.5 equiv.) and ammonium chloride (24.0 g, 449 mmol, 9.4 equiv.) in EtOH (500 mL) and water (100 mL) was stirred at 70° C. for 1 hour. The mixture was filtered through Celite®, rinsed with MeOH, and the solvent removed. The residue was triturated with water, filtered, washed with water, and dried to afford the title compound (7.40 g, 59% yield). LC-MS m/z 262/264 (M+H+).

### Step 4: 5-Bromo-6-chloro-3-cyclopropyl-3H-imidazo[4,5-b]pyridine

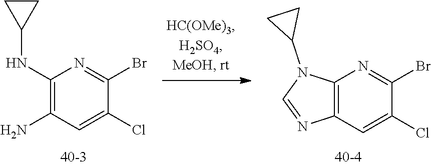 [SMILES: Nc1cc(Cl)c(Br)nc1NC1CC1 ; Clc1cc2ncn(C3CC3)c2nc1Br]

A mixture of 6-bromo-5-chloro-N2-cyclopropylpyridine-2,3-diamine 3 (7.40 g, 28.2 mmol, 1 equiv.), trimethyl orthoformate (7.63 mL, 69.7 mmol, 2.47 equiv.), and sulfuric acid (0.22 mL, 4.08 mmol, 0.14 equiv.) in MeOH (200 mL) was stirred at room temperature overnight. The reaction mixture was concentrated and subjected to flash column chromatography (0-60% EtOAc in hexanes) to afford the title compound (5.90 g, 77% yield). LC-MS m/z 272/274 (M+H⁺).

### Step 5: 6-Chloro-3-cyclopropyl-3H-imidazo[4,5-b]pyridine-5-carbonitrile

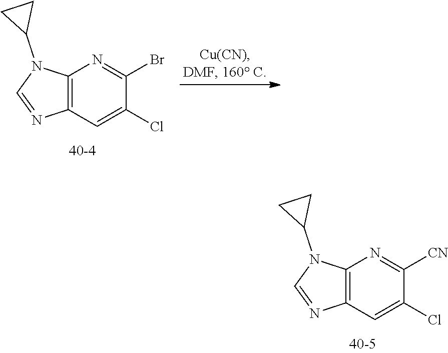 [SMILES: Clc1cc2ncn(C3CC3)c2nc1Br ; N#Cc1nc2c(cc1Cl)ncn2C1CC1]

A mixture of 5-bromo-6-chloro-3-cyclopropyl-3H-imidazo[4,5-b]pyridine (14.0 g, 51.4 mmol, 1 equiv.) and copper cyanide (5.60 g, 62.5 mmol, 1.22 equiv.) in DMF (170 mL) was sparged with N₂ and stirred at 160° C. overnight. The reaction mixture was cooled to 0° C., water (300 mL) was added, and the mixture was stirred vigorously. The slurry was filtered, the cake was washed with water, then the solid was added to a stirring mixture of ammonium hydroxide (300 mL) and EtOAc (300 mL). The mixture was filtered, the organic layer was collected, and the aqueous layer was extracted with EtOAc (250 mL). The organic layers were combined, washed with water (2×100 mL), brine (100 mL), dried over sodium sulfate, concentrated, and subjected to flash column chromatography (0-100% MeOH in DCM) to afforded the title compound (9.00 g, 80% yield). LC-MS m/z 219/221 (M+H⁺).

### Step 6: 6-Amino-3-cyclopropyl-3H-imidazo[4,5-b]pyridine-5-carbonitrile hydrochloride

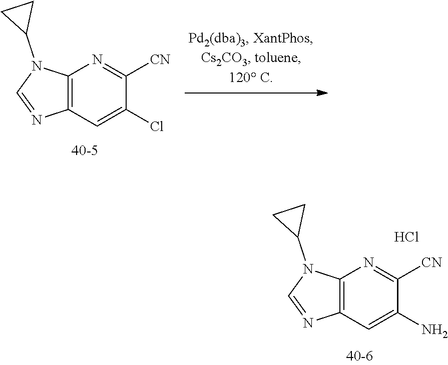 [SMILES: N#Cc1nc2c(cc1Cl)ncn2C1CC1 ; N#Cc1nc2c(cc1N)ncn2C1CC1 ; Cl]

A mixture of 6-chloro-3-cyclopropyl-3H-imidazo[4,5-b]pyridine-5-carbonitrile 5 (9.00 g, 41.2 mmol, 1 equiv.), cesium carbonate (30.0 g, 92.1 mmol, 2.24 equiv.), diphenylmethanimine (14.0 mL, 83.4 mmol, 2.02 equiv.), Pd₂(dba)₃ (6.00 g, 6.55 mmol, 0.16 equiv.) and XantPhos (7.60 g, 13.1 mmol, 0.38) in toluene (100 mL) was sparged with N₂ and stirred at 120° C. overnight. The reaction mixture was diluted in EtOAc, filtered, and the cake was washed with EtOAc until the filtrate was colorless. The combined filtrate was concentrated, dissolved in THF (200 mL), treated with aqueous HCl (1 M, 200 mL) and stirred at room temperature for 1 hour. The slurry was filtered, the cake was washed with EtOAc and dried overnight to afford the title compound (6.70 g, 69% yield). LC-MS m/z 200 (M+H⁺).

### Step 7: 6-Amino-7-bromo-3-cyclopropyl-3H-imidazo[4,5-b]pyridine-5-carbonitrile

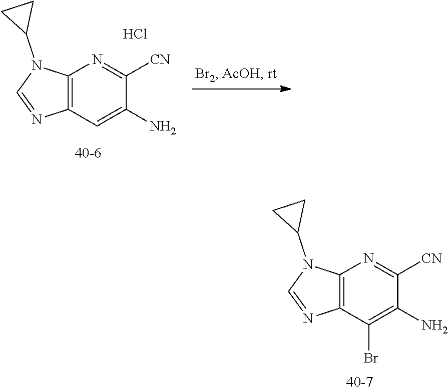 [SMILES: N#Cc1nc2c(cc1N)ncn2C1CC1 ; Cl ; N#Cc1nc2c(ncn2C2CC2)c(Br)c1N]

Bromine (10.0 mL, 195 mmol, 4.18 equiv.) was added to a stirring slurry of 6-amino-3-cyclopropyl-imidazo[4,5-b]pyridine-5-carbonitrile hydrochloride (11.0 g, 46.7 mmol, 1 equiv.) in glacial acetic acid (200 mL) at room temperature. The reaction mixture was stirred vigorously for 2 hours, concentrated, diluted with EtOAc (500 mL), and slowly added to a stirring solution of saturated sodium bicarbonate (1 L) at 0° C. The mixture was stirred for 30 minutes, quenched with aqueous NaOH (3 M) at 0° C. to pH 7-8, and the organic layer collected. The aqueous layer was further extracted with EtOAc (2×300 mL). The organic layers were combined, washed with saturated sodium bicarbonate (300 mL), brine (300 mL), dried over sodium sulfate, concentrated onto Celite®, and subjected to flash column chromatography (0-100% EtOAc in DCM) afforded the title compound (7.50 g, 58% yield). LC-MS m/z 278/280 (M+H⁺).

### Step 8: 6-Amino-7-(3-(benzyloxy)-2,6-dimethylphenyl)-3-cyclopropyl-3H-imidazo[4,5-b]pyridine-5-carbonitrile

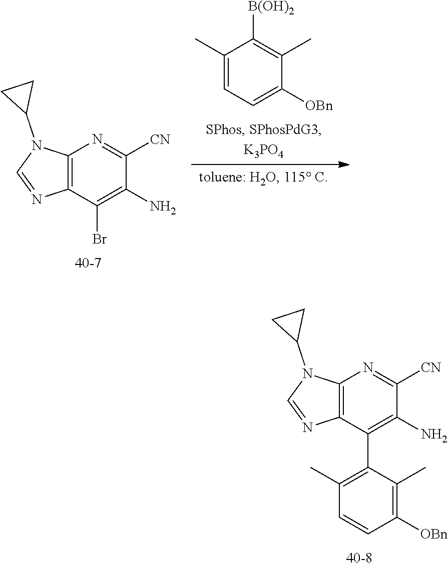 [SMILES: N#Cc1nc2c(ncn2C2CC2)c(Br)c1N ; Cc1ccc(OCc2ccccc2)c(C)c1B(O)O ; Cc1ccc(OCc2ccccc2)c(C)c1-c1c(N)c(C#N)nc2c1ncn2C1CC1]

A mixture of 6-amino-7-bromo-3-cyclopropyl-3H-imidazo[4,5-b]pyridine-5-carbonitrile (5.00 g, 18.0 mmol, 1 equiv.), (3-benzyloxy-2,6-dimethyl-phenyl)boronic acid (6.50 g, 25.4 mmol, 1.41 equiv.), potassium triphosphate (11.5 g, 54.2 mmol, 3.01 equiv.), SPhos (1.61 g, 3.57 mmol, 0.20 equiv.), and SPhosPdG₃ (1.41 g, 1.81 mmol, 0.10 equiv.) in toluene (100 mL) and water (10.0 mL) was stirred at 115° C. for 3 hours. The reaction mixture was filtered and washed with EtOAc, and the organic layer was separated. The aqueous layer was extracted with EtOAc (2×100 mL). The combined organic layers were washed with water (100 mL), brine (100 mL), dried over magnesium sulfate, concentrated and subjected to flash column chromatography (0-100% EtOAc in DCM) to afford the title compound (4.00 g, 54% yield). LC-MS m/z 410 (M+H⁺).

### Step 9: 6-Amino-7-(3-(benzyloxy)-2,6-dimethylphenyl)-3-cyclopropyl-3H-imidazo[4,5-b]pyridine-5-carboxamide

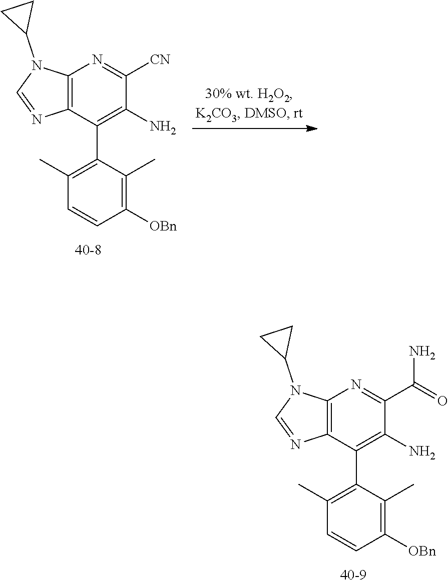 [SMILES: Cc1ccc(OCc2ccccc2)c(C)c1-c1c(N)c(C#N)nc2c1ncn2C1CC1 ; Cc1ccc(OCc2ccccc2)c(C)c1-c1c(N)c(C(N)=O)nc2c1ncn2C1CC1]

Hydrogen peroxide (30% wt., 22.0 mL, 194 mmol, 20 equiv.) was added to a mixture of 6-amino-7-(3-benzyloxy-2,6-dimethyl-phenyl)-3-cyclopropyl-imidazo[4,5-b]pyridine-5-carbonitrile (4.00 g, 9.77 mmol, 1 equiv.) and potassium carbonate (27.0 g, 195 mmol, 20 equiv.) in DMSO (150 mL) at room temperature and stirred for 2 hours. The reaction mixture was diluted water (200 mL) and extracted with EtOAc (300 mL). The organic layer was washed with water (200 mL), brine (200 mL), dried over sodium sulfate, concentrated onto Celite®), and subjected to flash column chromatography (0-20% MeOH in DCM) to afford the title (3.00 g, 72% yield). LC-MS m/z 428 (M+H⁺).

### Step 10: (P)-6-amino-3-cyclopropyl-7-(3-hydroxy-2,6-dimethylphenyl)-3H-imidazo[4,5-b]pyridine-5-carboxamide. (77)

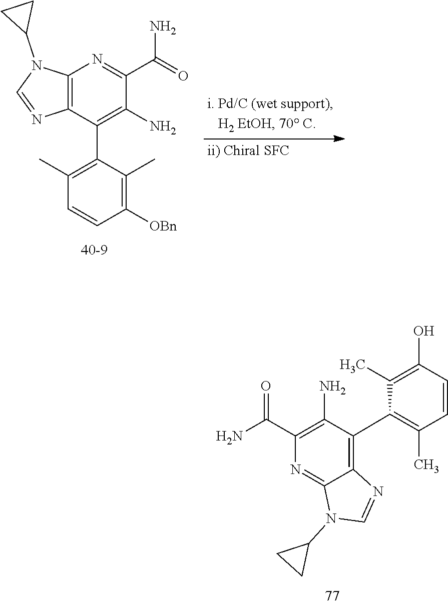

A mixture of 6-amino-7-(3-benzyloxy-2,6-dimethyl-phenyl)-3-cyclopropyl-imidazo[4,5-b]pyridine-5-carboxamide (3.00 g, 7.02 mmol) and palladium on carbon (10% wt. wet support, 67% water, 5.60 g) in ethanol (150 mL) was stirred under hydrogen gas (1 atm) at 70° C. for 1 hour. The reaction mixture was filtered through Celite®, rinsed with MeOH, the filtrate was concentrated onto Celite®, and subjected to flash column chromatography (0-15% MeOH in DCM) to afford the crude 6-amino-3-cyclopropyl-7-(3-hydroxy-2,6-dimethylphenyl)-3H-imidazo[4,5-b]pyridine-5-carboxamide (2.00 g). The crude material was submitted for SFC chiral separation: (Instrument: Waters SFC Prep 150 Mgm; Column: Chiralcel OJ, 30×150 mm, 5 μm; Conditions: Isocratic at 20% methanol with 80% CO₂; Flow Rate: 100 mL/min) to provide the title compound (peak 1, 1.41 minutes, 860 mg, 36% yield). LCMS: m/z 338 (M+H⁺). ¹H NMR (400 MHz, DMSO) δ 9.23 (s, 1H), 8.27 (s, 1H), 8.01 (d, J=3.4 Hz, 1H), 7.56 (d, J=3.4 Hz, 1H), 6.98 (d, J=8.2 Hz, 1H), 6.80 (d, J=8.2 Hz, 1H), 5.85-5.80 (m, 2H), 3.61 (tt, J=7.4, 4.0 Hz, 1H), 1.75 (s, 3H), 1.68 (s, 3H), 1.19-1.02 (m, 4H).

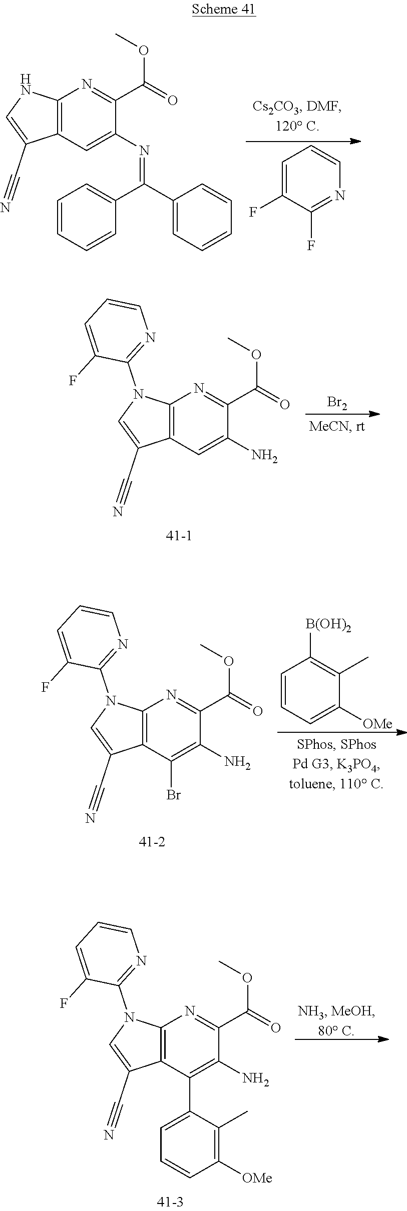

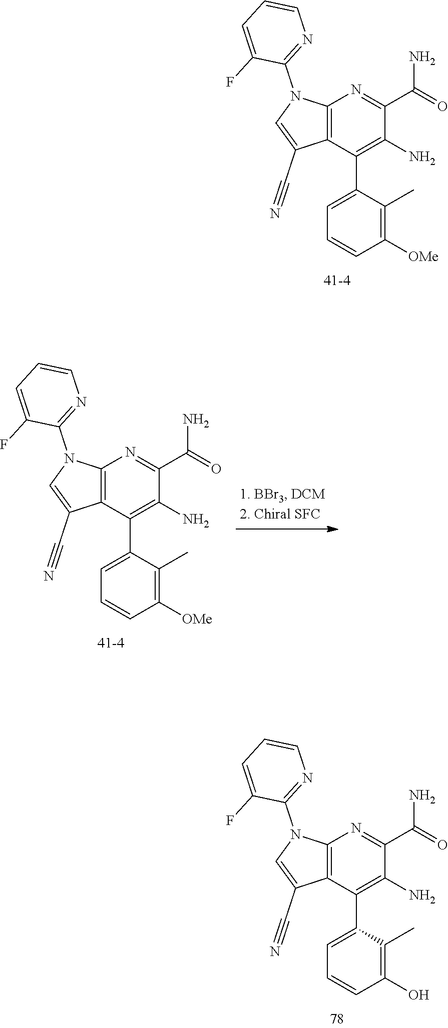

### Example 67. (M)-5-Amino-3-cyano-1-(3-fluoropyridin-2-yl)-4-(3-hydroxy-2-methylphenyl)-1H-pyrrolo[2,3-b]pyridine-6-carboxamide (78)

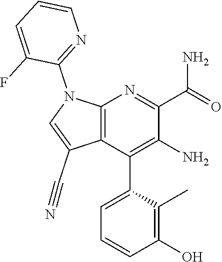

### Step 1: Methyl 5-amino-3-cyano-1-(3-fluoropyridin-2-yl)-1H-pyrrolo[2,3-b]pyridine-6-carboxylate

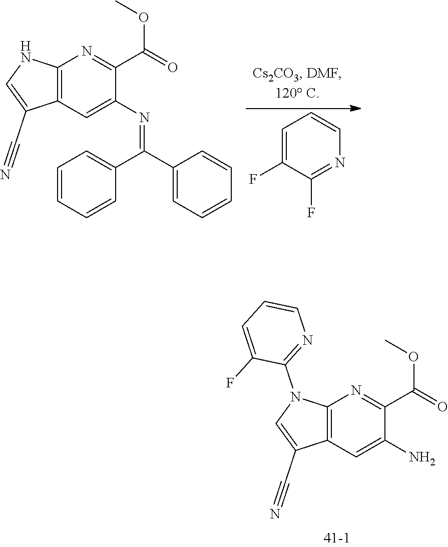

2,3-Difluoropyridine (0.50 mL, 5.50 mmol, 3.5 equiv.) was added to a solution of methyl 5-(benzhydrylideneamino)-3-cyano-1H-pyrrolo[2,3-b]pyridine-6-carboxylate (600 mg, 1.58 mmol, 1 equiv.) and cesium carbonate (1.60 g, 4.91 mmol, 3.1 equiv.) in DMF (10 mL). The mixture was sparged with nitrogen and stirred vigorously at 120° C. overnight. The reaction mixture was diluted with water and EtOAc, acidified to pH ~1 with conc. HCl, and filtered. The organic layer was collected and the aqueous layer was extracted with EtOAc. The combined organic layers were washed with brine, dried over sodium sulfate, and concentrated. The residue was suspended in MeOH (10 mL), treated with concentrated sulfuric acid (2 mL), stirred at 80° C. for 1 hour. The reaction mixture was concentrated, redissolved in EtOAc, cooled on ice, and aqueous NaOH (3 M) was added to pH ~7. The mixture was extracted with EtOAc (2×30 mL). The organic layers were combined, washed with brine (10 mL), dried over magnesium sulfate and concentrated to afford the title compound (300 mg, 61% yield). LC-MS m/z 312 (M+H⁺).

### Step 2: Methyl 5-amino-4-bromo-3-cyano-1-(3-fluoropyridin-2-yl)-1H-pyrrolo[2,3-b]pyridine-6-carboxylate

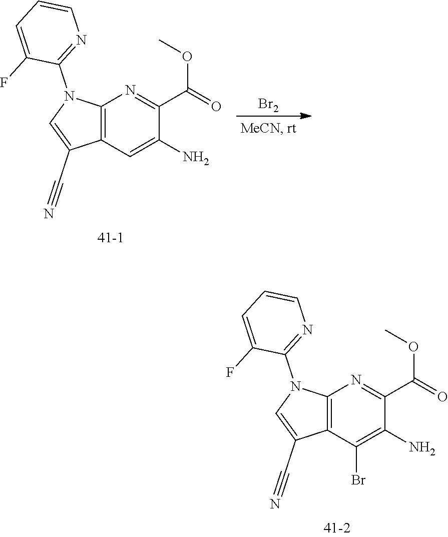

Bromine (0.30 mL, 5.90 mmol, 6.15 equiv.) in acetonitrile (20 mL) was added to a stirring solution of methyl 5-amino-3-cyano-1-(3-fluoro-2-pyridyl)pyrrolo[2,3-b]pyridine-6-carboxylate (300 mg, 0.96 mmol, 1 equiv.) in acetonitrile (20 mL) at room temperature and the reaction mixture was stirred for 30 minutes. The reaction mixture was diluted with EtOAc and slowly added to a stirring aqueous solution of sodium thiosulfate (10% w/v) at 0° C. and allowed to stir at room temperature for 15 minutes. The organic layer was collected, the aqueous layer was extracted with EtOAc. The combined organic layers were washed with brine, dried over sodium sulfate, concentrated, and subjected to flash column chromatography (0-100% EtOAc in DCM) to afford the title compound (270 mg, 72% yield). LC-MS m/z 390/392 (M+H⁺).

### Step 3: Methyl 5-amino-3-cyano-1-(3-fluoropyridin-2-yl)-4-(3-methoxy-2-methylphenyl)-1H-pyrrolo[2,3-b]pyridine-6-carboxylate

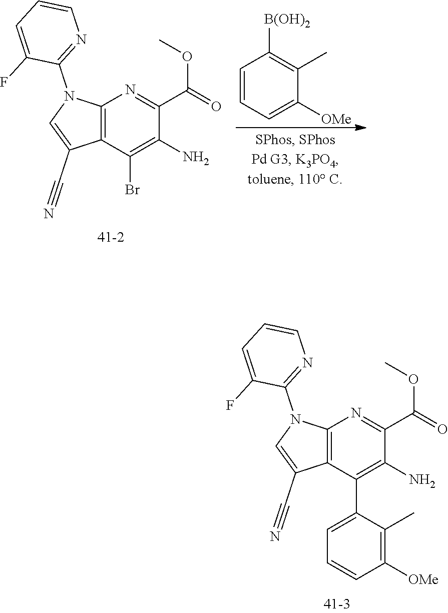

A mixture of methyl 5-amino-4-bromo-3-cyano-1-(3-fluoro-2-pyridyl)pyrrolo[2,3-b]pyridine-6-carboxylate (190 mg, 0.49 mmol, 1 equiv.) potassium phosphate (420 mg, 1.98 mmol, 4 equiv.), SPhosPdG3 (45 mg, 0.06 mmol, 0.1 equiv.). (3-methoxy-2-methyl-phenyl)boronic acid (170 mg, 1.02 mmol, 2.1 equiv.), and SPhos (45 mg, 0.11 mmol, 0.22 equiv.) in toluene (10.0 mL) was stirred at 100° C. overnight. The reaction mixture was diluted with aqueous HCl (1 M) and EtOAc, the organic layer was collected, the aqueous layer was further extracted with EtOAc. The combined organic layers were dried over sodium sulfate, concentrated, and subjected to flash column chromatography (0-100% EtOAc in hexanes) to afford the title compound (180 mg, 86% yield). LC-MS m/z 432 (M+H⁺).

### Step 4: 5-Amino-3-cyano-1-(3-fluoropyridin-2-yl)-4-(3-methoxy-2-methylphenyl)-1H-pyrrolo[2,3-b]pyridine-6-carboxamide

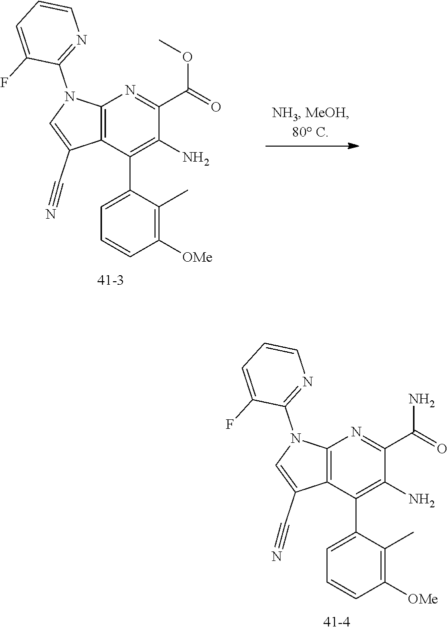 [SMILES: COC(=O)c1nc2c(c(C#N)cn2-c2ncccc2F)c(-c2cccc(OC)c2C)c1N ; COc1cccc(-c2c(N)c(C(N)=O)nc3c2c(C#N)cn3-c2ncccc2F)c1C]

A solution of methyl 5-amino-3-cyano-1-(3-fluoro-2-pyridyl)-4-(3-methoxy-2-methyl-phenyl)pyrrolo[2,3-b]pyridine-6-carboxylate (180 mg, 0.42 mmol, 1 equiv.) in an ammonia solution in MeOH (7 M, 12.0 mL, 84.0 mmol, 200 equiv.) was stirred at 80° C. overnight. The solution was concentrated to afford the title compound (170 mg, 98% yield). LC-MS m/z 417 (M+H⁺).

### Step 5: (R)-5-Amino-3-cyano-1-(3-fluoropyridin-2-yl)-4-(3-hydroxy-2-methylphenyl)-1H-pyrrolo[2,3-b]pyridine-6-carboxamide. (78)

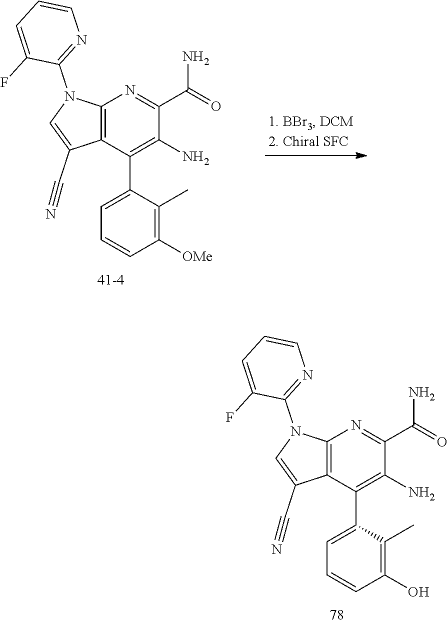

A boron tribromide solution in DCM (1 M, 4.00 mL, 4.00 mmol, 9.8 equiv.) was added slowly to a solution of 5-amino-3-cyano-1-(3-fluoro-2-pyridyl)-4-(3-methoxy-2-methyl-phenyl)pyrrolo[2,3-b]pyridine-6-carboxamide (170 mg, 0.41 mmol, 1 equiv.) in DCM (10 mL) and the reaction mixture was stirred at room temperature for 3 hours. The reaction mixture was slowly added to a stirring mixture of saturated aqueous sodium bicarbonate and EtOAc at 0° C. and the resulting mixture was allowed to warm to room temperature. The organic layer was washed with brine, dried over magnesium sulfate, and concentrated. The residue was triturated with minimal acetonitrile, filtered, rinsed with acetonitrile, and dried. The solid was in DMF, filtered, and submitted for SFC chiral separation: (Instrument: Waters SFC Prep 150 Mgm; Column: Chiralcel OJ, 30×150 mm, 5 um; Conditions: Isocratic at 20% methanol with 80% CO₂; Flow Rate: 100 mL/min) provided the title compound (peak 1, 2.74 min, 58 mg, 35% yield). LCMS: m/z 403 (M+H⁺). ¹H NMR (400 MHz, DMSO) δ 9.55 (s, 1H), 8.76 (s, 1H), 8.54-8.48 (m, 1H), 8.13 (t, J=9.1 Hz, 1H), 7.76-7.65 (m, 2H), 7.57 (s, 1H), 7.16 (t, J=7.8 Hz, 1H), 6.93 (d, J=8.0 Hz, 1H), 6.68 (d, J=7.5 Hz, 1H), 6.11 (s, 2H), 1.86 (s, 3H). ¹⁹F NMR (400 MHz, DMSO) δ 123.19.

### Example 68. (P)- and (M)-3-cyano-1-ethyl-4-(3-hydroxy-2,6-dimethyl-phenyl)pyrrolo[2,3-b]pyridine-6-carboxamide (79 and 80)

### Step 1: 4-Bromo-7-oxido-1H-pyrrolo[2,3-b]pyridin-7-ium

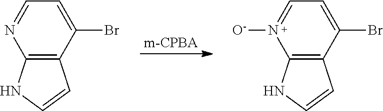

To a solution of 4-bromo-1H-pyrrolo[2,3-b]pyridine (20 g×2, 101 mmol×2, 1 eq) in DCM (500 mL) was added m-CPBA (30.9 g×2, 152 mmol×2, 85% purity, 1.5 eq). The mixture was stirred at 20° C. for 2 hr. The reaction mixture was quenched by addition of aqueous saturated Na₂SO₃ (500 mL) at 25° C. for 1 hour, and then the residue was extracted with DCM (1000 mL×3). The combined organic layers were washed with aqueous saturated Na₂SO₃ (2000 mL×2), dried over anhydrous Na₂SO₄, filtered, and concentrated under reduced pressure to provide a crude residue. The residue was triturated with MeCN (500 mL) at 25° C. for 12 hr to give 4-bromo-7-oxido-1H-pyrrolo[2,3-b]pyridin-7-ium (40 g, crude). LCMS: m/z 213.0 [M+H]⁺.

### Step 2: 4-Bromo-1H-pyrrolo[2,3-b]pyridine-6-carbonitrile

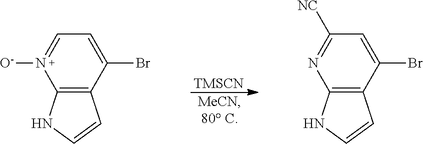

To a solution of 4-bromo-7-oxido-1H-pyrrolo[2,3-b]pyridin-7-ium (40 g, 187 mmol, 1 eq) in MeCN (500 mL) was added TMSCN (95.1 g, 959 mmol, 120 mL, 5.11 eq). After stirring at 80° C. for 72 hours, the reaction mixture was concentrated under reduced pressure to afford a residue which was diluted with H₂O (500 mL) and extracted with EtOAc (500 mL×3). The combined organic layers were washed with aqueous saturated NaCl (1000 mL×2), dried over anhydrous Na₂SO₄, filtered, and concentrated. The crude product was triturated with MeCN (500 mL) at 20° C. for 2 hours to give 4-bromo-1H-pyrrolo[2,3-b]pyridine-6-carbonitrile (40 g, crude). LCMS: m/z 222/224. [M+H]⁺.

### Step 3: 4-Bromo-1-ethyl-pyrrolo[2,3-b]pyridine-6-carbonitrile

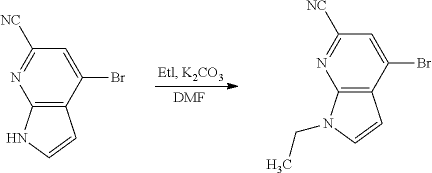

To a solution of 4-bromo-1H-pyrrolo[2,3-b]pyridine-6-carbonitrile (30 g, 135 mmol, 1 eq) in DMF (200 mL) were added K₂CO₃ (56.0 g, 405 mmol, 3 eq) and EtI (25.3 g, 162 mmol, 12.9 mL, 1.2 eq). The mixture was stirred at 25° C. for 16 hours. The reaction mixture was diluted with H₂O (200 mL) and extracted with EtOAc (300 mL×3). The combined organic layers were washed with brine (200 mL×3), dried over Na₂SO₄, filtered, and concentrated under reduced pressure to afford a residue. The residue was purified by column chromatography on silica gel (0%-30% ethyl acetate in petroleum ether) to afford 4-bromo-1-ethyl-pyrrolo[2,3-b]pyridine-6-carbonitrile (26 g, 103 mmol, 77% yield). ¹H NMR: (400 MHz, DMSO-d₆) δ=8.09 (d, 1H), 8.03 (s, 1H), 6.63 (d, 1H), 4.33 (q, 2H), 1.40 (t, 3H).

### Step 4. 4-Bromo-1-ethyl-pyrrolo[2,3-b]pyridine-6-carboxylic acid

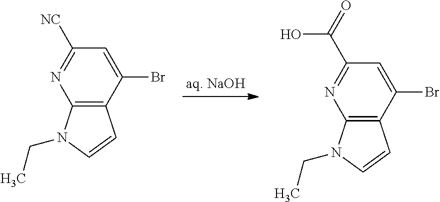

A solution of 4-bromo-1-ethyl-pyrrolo[2,3-b]pyridine-6-carbonitrile (26 g, 103 mmol, 1 eq) in aqueous NaOH (200 mL, 2 M) was stirred at 80° C. for 16 hours. The reaction mixture was filtered and concentrated to give 4-bromo-1-ethyl-pyrrolo[2,3-b]pyridine-6-carboxylic acid (26 g, crude). LCMS: m/z 269/271 [M+H]⁺.

### Step 5: Methyl 4-bromo-1-ethyl-pyrrolo[2,3-b]pyridine-6-carboxylate

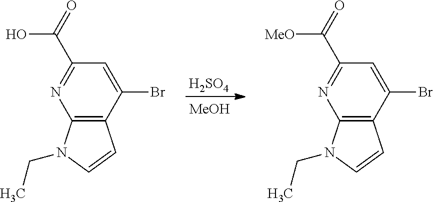

To a solution of 4-bromo-1-ethyl-pyrrolo[2,3-b]pyridine-6-carboxylic acid (26 g, 96.6 mmol, 1 eq) in MeOH (200 mL) was added H₂SO₄ (25 mL, 5 eq). The mixture was stirred at 80° C. for 2 hr. The reaction mixture was adjusted pH to 7 with aqueous NaHCO₃ (2 M), and then the residue was extracted with EtOAc (300 mL×2). The organic phase was dried over anhydrous Na₂SO₄, filtered and concentrated to give methyl 4-bromo-1-ethyl-pyrrolo[2,3-b]pyridine-6-carboxylate (24 g, crude). LCMS: m/z 283/285 [M+H]⁺. ¹H NMR: (400 MHz, DMSO-d₆) δ=8.00 (d, 1H), 7.98 (s, 1H), 6.56 (d, 1H), 4.34 (q, 2H), 3.90 (s, 3H), 1.40 (t, 3H).

### Step 6: Methyl 4-bromo-3-cyano-1-ethyl-pyrrolo[2,3-b]pyridine-6-carboxylate

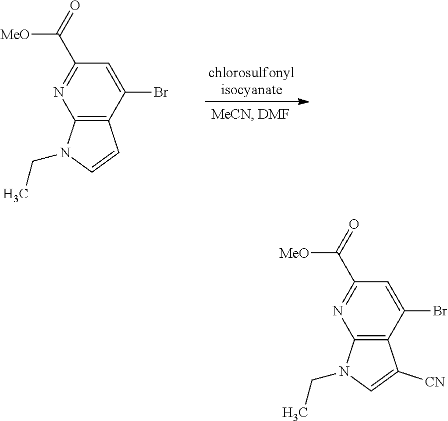 [SMILES: CCn1ccc2c(Br)cc(C(=O)OC)nc21 ; CCn1cc(C#N)c2c(Br)cc(C(=O)OC)nc21]

To a solution of methyl 4-bromo-1-ethyl-pyrrolo[2,3-b]pyridine-6-carboxylate (24 g, crude) in DMF (50 mL) was added a solution N-(oxomethylene)sulfamoyl chloride (60 g, 424 mmol, 37 mL, 5 eq) in MeCN (257 mL) at 0° C. The mixture was stirred at 0° C. for 0.5 hour. The reaction mixture was quenched with H₂O (50 mL) and extracted with EtOAc (50 mL×3). The combined organic layers were washed with brine (50 mL×3), dried over Na₂SO₄, filtered, and concentrated to give a residue. The crude product was triturated with MeCN (50 mL) to give methyl 4-bromo-3-cyano-1-ethyl-pyrrolo[2,3-b]pyridine-6-carboxylate (16 g, 46.7 mmol, 55% yield, 90% purity). LCMS: m/z 308/310 [M+H]⁺. ¹H NMR: (400 MHz, CD₃OD) δ=8.57 (s, 1H), 8.23 (s, 1H), 4.50 (q, 2H), 4.02 (s, 3H), 1.52 (t, 3H).

### Step 7: Methyl 4-(3-benzyloxy-2,6-dimethyl-phenyl)-3-cyano-1-ethyl-pyrrolo[2,3-b]pyridine-6-carboxylate

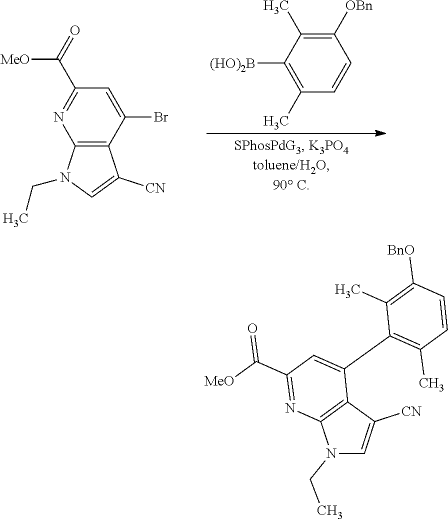 [SMILES: CCn1cc(C#N)c2c(Br)cc(C(=O)OC)nc21 ; Cc1ccc(OCc2ccccc2)c(C)c1B(O)O ; CCn1cc(C#N)c2c(-c3c(C)ccc(OCc4ccccc4)c3C)cc(C(=O)OC)nc21]

To a solution of methyl 4-bromo-3-cyano-1-ethyl-pyrrolo[2,3-b]pyridine-6-carboxylate (10 g, 32.45 mmol, 1 eq) and 3-benzyloxy-2,6-dimethyl-phenyl)boronic acid (9.97 g, 38.94 mmol, 1.2 eq) in a mixture of toluene (150 mL) and H₂O (17 mL), were added SPhos Pd G₃ (2.53 g, 3.25 mmol, 0.1 eq), SPhos (2.66 g, 6.48 mmol, 0.2 eq) and K₃PO₄ (20.67 g, 97.36 mmol, 3 eq). The mixture was stirred at 90° C. under N₂ for 17 hours. The reaction mixture was concentrated to give a residue, which was diluted with water (150 mL), followed by extraction with EtOAc (150 mL×3). The organic phase was washed with aqueous saturated NaCl (50 mL×2), dried over anhydrous Na₂SO₄, filtered, and concentrated under vacuum. The crude product was purified by flash silica gel chromatography (ISCOR; 40 g SepaFlash® Silica Flash Column, eluent of 0~54% ethyl acetate/petroleum ether gradient at 30 mL/min) to give methyl 4-(3-benzyloxy-2,6-dimethyl-phenyl)-3-cyano-1-ethyl-pyrrolo[2,3-b]pyridine-6-carboxylate (9.13 g, 19.11 mmol, 59% yield, 92% purity). LCMS: m/z 440.2 [M+H]⁺

### Step 8: 4-(3-Benzyloxy-2,6-dimethyl-phenyl)-3-cyano-1-ethyl-pyrrolo[2,3-b]pyridine-6-carboxylic acid

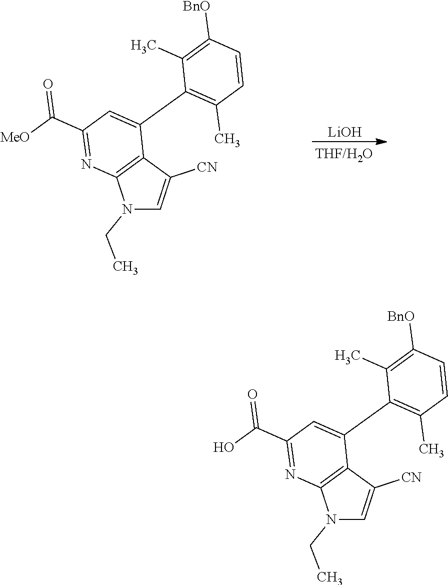 [SMILES: CCn1cc(C#N)c2c(-c3c(C)ccc(OCc4ccccc4)c3C)cc(C(=O)OC)nc21 ; "LiOH" ; CCn1cc(C#N)c2c(-c3c(C)ccc(OCc4ccccc4)c3C)cc(C(=O)O)nc21]

To a solution of methyl 4-(3-benzyloxy-2, 6-dimethyl-phenyl)-3-cyano-1-ethyl-pyrrolo[2,3-b]pyridine-6-carboxylate (19 g, 43.23 mmol, 1 eq) in THF (200 mL) was added a solution of LiOH·H₂O (5.4 g, 129.69 mmol, 3 eq) in H₂O (200 mL). The mixture was stirred at 15° C. for 18 hours. The reaction mixture was concentrated to reduce the volume of THF, then the pH was adjusted to around 3 with aqueous HCL (1 M) at 0° C. The mixture was filtered and concentrated to give the crude product. The crude product was triturated with MeCN (200 mL) at 15° C. for 2 hours to give 4-(3-benzyloxy-2,6-dimethyl-phenyl)-3-cyano-1-ethyl-pyrrolo[2,3-b]pyridine-6-carboxylic acid (16.6 g, 39.01 mmol, 90% yield, 100% purity). LCMS: m/z 426.2 [M+H]⁺. ¹H NMR: (400 MHz, DMSO-d₆) δ=8.75 (s, 1H), 7.69 (s, 1H), 7.44-7.55 (m, 2H), 7.39 (t, 2H), 7.33 (d, 1H), 7.11-7.18 (m, 1H), 7.02-7.10 (m, 1H), 5.09-5.22 (m, 2H), 4.45 (m, 2H), 1.83 (s, 3H), 1.80 (s, 3H), 1.50 (t, 3H).

### Step 9: 4-(3-Benzyloxy-2,6-dimethyl-phenyl)-3-cyano-1-ethyl-pyrrolo[2,3-b]pyridine-6-carboxamide

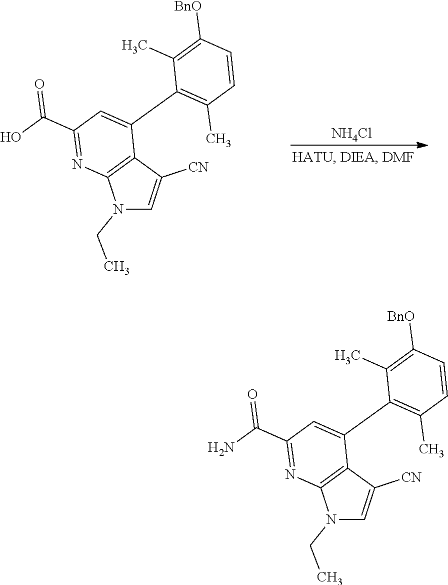 [SMILES: CCn1cc(C#N)c2c(-c3c(C)ccc(OCc4ccccc4)c3C)cc(C(=O)O)nc21 ; "NH4Cl" ; CCn1cc(C#N)c2c(-c3c(C)ccc(OCc4ccccc4)c3C)cc(C(N)=O)nc21]

To a solution of 4-(3-benzyloxy-2,6-dimethyl-phenyl)-3-cyano-1-ethyl-pyrrolo[2,3-b]pyridine-6-carboxylic acid (15 g, 35.25 mmol, 1 eq) and HATU (17.4 g, 45.83 mmol, 1.3 eq) in DMF (120 mL) were added DIEA (13.6 g, 105.76 mmol, 3 eq) and NH₄Cl (5.7 g, 105.76 mmol, 3 eq). The mixture was stirred at 15° C. for 4 hours. The reaction mixture was diluted with H₂O (1.5 L), then filtered to provide a filter cake. The crude product was triturated with MeCN (30 mL) at 15° C. for 2 hours to give 4-(3-benzyloxy-2,6-dimethyl-phenyl)-3-cyano-1-ethyl-pyrrolo[2,3-b]pyridine-6-carboxamide (13 g, 30.62 mmol, 88% yield). LCMS: m/z 425.3 [M+H]⁺. ¹H NMR: (400 MHz, DMSO-d₆) δ=8.71 (s, 1H), 8.35 (d, 1H), 7.57-7.79 (m, 2H), 7.48 (d, 2H), 7.39 (t, 2H), 7.25-7.35 (m, 1H), 7.11-7.18 (m, 1H), 7.04-7.10 (m, 1H), 5.09-5.21 (m, 2H), 4.51 (m, 2H), 1.81 (d, 6H), 1.50 (t, 3H).

### Step 10: 3-Cyano-1-ethyl-4-(3-hydroxy-2,6-dimethyl-phenyl)pyrrolo[2,3-b]pyridine-6-carboxamide

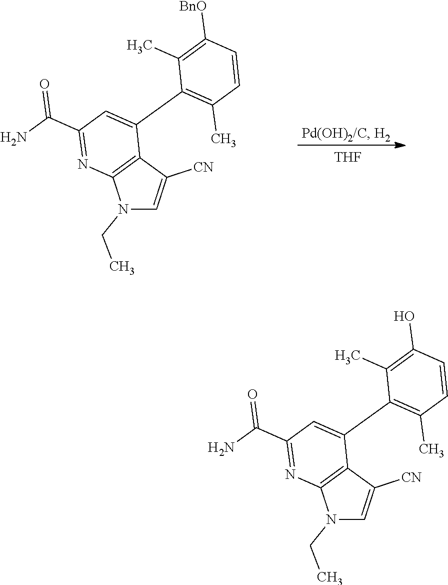 [SMILES: CCn1cc(C#N)c2c(-c3c(C)ccc(OCc4ccccc4)c3C)cc(C(N)=O)nc21 ; "Pd(OH)2/C, H2" ; CCn1cc(C#N)c2c(-c3c(C)ccc(O)c3C)cc(C(N)=O)nc21 ; "THF"]

To a solution of 4-(3-benzyloxy-2, 6-dimethyl-phenyl)-3-cyano-1-ethyl-pyrrolo[2,3-b]pyridine-6-carboxamide (17 g, 40.05 mmol, 1 eq) in THF (300 mL) was added Pd(OH)₂/C (4.2 g, 5.98 mmol, 20% purity, 0.15 eq). The mixture was degassed with H₂ and stirred at 15° C., 15 Psi for 3 hours. The reaction mixture was filtered to give the filtrate, which was concentrated directly to give the residue. The crude product was triturated with MeCN/H₂O (1:2, 60 mL) at 15° C. for 2 hours to give 3-cyano-1-ethyl-4-(3-hydroxy-2,6-dimethyl-phenyl)pyrrolo[2,3-b]pyridine-6-carboxamide (11.2 g, 32.16 mmol, 80% yield, 96% purity). LCMS: m/z 335.0 [M+H]⁺. ¹H NMR: (400 MHz, DMSO-d₆) δ=9.23-9.33 (m, 1H), 8.65-8.72 (m, 1H), 8.29-8.41 (m, 1H), 7.68-7.81 (m, 1H), 7.59-7.66 (m, 1H), 6.92-6.99 (m, 1H), 6.77-6.85 (m, 1H), 4.50 (m, 2H), 1.64-1.81 (d, 6H), 1.44-1.53 (t, 3H).

### Step 11: Chiral Separation

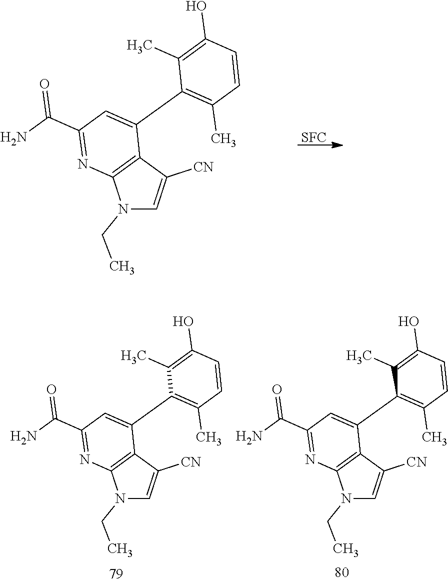

22.1 g of racemate was separated by SFC to provide 79: (S)-3-cyano-1-ethyl-4-(3-hydroxy-2,6-dimethyl-phenyl)pyrrolo[2,3-b]pyridine-6-carboxamide (7.71 g, 22.64 mmol, 34% yield, 98.24% purity) and 80: (R)-3-cyano-1-ethyl-4-(3-hydroxy-2,6-dimethyl-phenyl)pyrrolo[2,3-b]pyridine-6-carboxamide (8.26 g, 24.03 mmol, 36% yield, 97.27% purity) and. Conditions: column: CHIRALPAK AD 50×4.6 mm I.D., 3 μm. Mobile phase A: CO₂; Mobile phase B: ethanol (0.05% DEA); Gradient: from 5% to 40% of B in 2.5 min and hold 40% for 0.5 min. then 5% of B for 1 min; Flow rate: 4 mL/min; Column temp.: 35° C., ABPR: 1500 psi.

79. LCMS: m/z 335.2 [M+H]⁺. ¹H NMR: (400 MHz, DMSO-d₆) δ=9.24 (s, 1H), 8.69 (s, 1H), 8.33 (s, 1H), 7.71 (s, 1H), 7.64 (s, 1H), 6.96 (d, 1H), 6.81 (d, 1H), 4.51 (m, 2H), 1.77 (s, 3H), 1.70 (s, 3H), 1.49 (t, 3H). SFC: t_{R}=2.760

80. LCMS: m/z 335.2 [M+H]⁺. ¹H NMR: (400 MHz, DMSO-d₆) δ=9.25 (s, 1H), 8.69 (s, 1H), 8.24-8.46 (m, 1H), 7.68-7.78 (m, 1H), 7.63 (s, 1H), 6.92-7.00 (d, 1H), 6.77-6.86 (d, 1H), 4.45-4.58 (m, 2H), 1.77 (s, 3H), 1.70 (s, 3H), 1.49 (t, 3H). SFC: t_{R}=1.651.

### Example 69. (P)-6-amino-3-ethyl-7-(3-hydroxy-2,6-dimethylphenyl)-3H-imidazo[4,5-b]pyridine-5-carboxamide (81)

### Step 1: 6-(Ethylamino)-3,5-dinitropyridine-2-carbonitrile

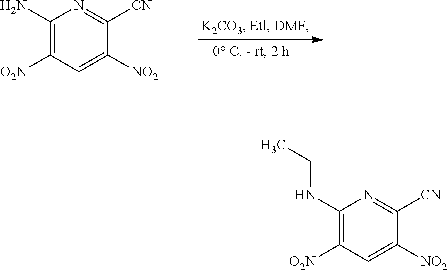 [SMILES: N#Cc1nc(N)c([N+](=O)[O-])cc1[N+](=O)[O-] ; CCNc1nc(C#N)c([N+](=O)[O-])cc1[N+](=O)[O-]]

To a stirred solution of 6-amino-3,5-dinitropyridine-2-carbonitrile (4 g, 19.128 mmol, 1 equiv) in DMF (48 mL) was added K₂CO₃ (2.64 g, 19.128 mmol, 1 equiv). Iodoethane (3.82 mL, 47.820 mmol, 1.2 equiv) was added dropwise to the mixture at 0° C. The resulting mixture was stirred for an additional 2 h at room temperature. The resulting mixture was extracted with EtOAc (3×200 mL). The combined organic layers were washed with water (3×200 mL) and dried over anhydrous Na₂SO₄. After filtration, the filtrate was concentrated under reduced pressure. The residue was purified by silica gel column chromatography (petroleum ether (PE)/ethyl acetate (EA) 9:1) to afford 6-(ethylamino)-3,5-dinitropyridine-2-carbonitrile (1.7 g, 37.47%).

### Step 2: 3,5-Diamino-6-(ethylamino) pyridine-2-carbonitrile

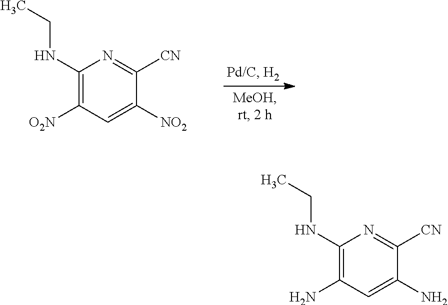 [SMILES: CCNc1nc(C#N)c([N+](=O)[O-])cc1[N+](=O)[O-] ; CCNc1nc(C#N)c(N)cc1N]

To a solution of 6-(ethylamino)-3,5-dinitropyridine-2-carbonitrile (1.7 g, 7.168 mmol, 1 equiv) in MeOH (100 mL) was added Pd/C (1.70 g, 15.985 mmol, 2.23 equiv). The mixture was hydrogenated at room temperature for 2 h using a hydrogen-filled balloon, filtered through a Celite® pad, and concentrated under reduced pressure. The crude product was used in the next step without purification.

### Step 3: 6-Amino-3-ethylimidazo[4,5-b]pyridine-5-carbonitrile

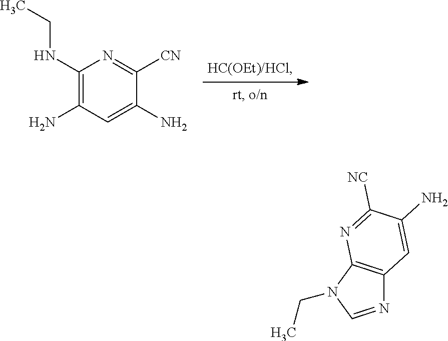

To a solution of 3,5-diamino-6-(ethylamino) pyridine-2-carbonitrile (500 mg, 2.821 mmol, 1 equiv) in triethyl orthoformate (8 mL) was added HCl (0.2 mL). The mixture was stirred overnight and then basified to pH 8 with saturated NaHCO₃ (aqueous). The resulting mixture was extracted with EtOAc (3×30 mL). The combined organic layers were washed with water (3×30 mL) and dried over anhydrous Na₂SO₄. After filtration, the filtrate was concentrated under reduced pressure. The residue was purified by silica gel column chromatography (PE/EA 1:1) to afford 6-amino-3-ethylimidazo[4,5-b]pyridine-5-carbo-nitrile (300 mg, 56.80%).

### Step 4: 6-Amino-7-bromo-3-ethylimidazo[4,5-b]pyridine-5-carbonitrile

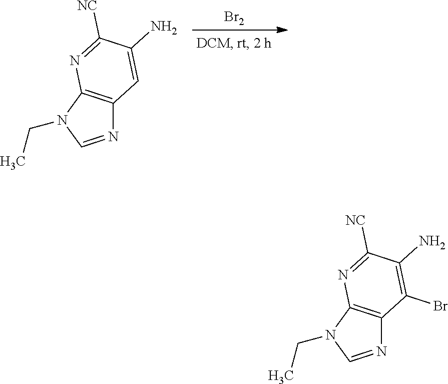

To a solution of 6-amino-3-ethylimidazo[4,5-b]pyridine-5-carbonitrile (105 mg, 0.561 mmol, 1 equiv) in DCM (2 mL) was added Br₂ (0.03 mL, 0.673 mmol, 1.2 equiv) at 0° C. The mixture was stirred for 2 h at room temperature. The reaction was quenched with MeOH at room temperature. The residue was purified by preparative-scale TLC (CH₂Cl₂/MeOH 15:1) to afford 6-amino-7-bromo-3-ethylimidazo[4,5-b]pyridine-5-carbonitrile (90 mg, 60.30%).

### Step 5: 6-Amino-3-ethyl-7-(3-methoxy-2,6-dimethylphenyl)imidazo[4,5-b]pyridine-5-carbonitrile

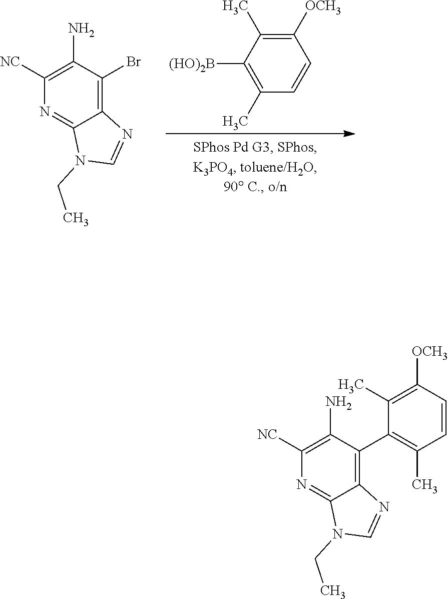 [SMILES: CCn1cnc2c(Br)c(N)c(C#N)nc21 ; COc1ccc(C)c(B(O)O)c1C ; CCn1cnc2c(-c3c(C)ccc(OC)c3C)c(N)c(C#N)nc21]

To a solution of 6-amino-7-bromo-3-ethylimidazo[4,5-b]pyridine-5-carbonitrile (80 mg, 0.301 mmol, 1 equiv) and 3-methoxy-2,6-dimethylphenylboronic acid (64.94 mg, 0.361 mmol, 1.2 equiv) in toluene (3 mL) and H₂O (0.6 mL) were added K₃PO₄ (191.44 mg, 0.903 mmol, 3 equiv) and SPhos Pd Gen.3 (23.46 mg, 0.030 mmol, 0.1 equiv). After stirring overnight at 90° C. under nitrogen, the mixture was extracted with EtOAc (3×30 mL). The combined organic layers were washed with water (3×30 mL) and dried over anhydrous Na₂SO₄. After filtration, the filtrate was concentrated under reduced pressure. The residue was purified by reversed-phase flash chromatography: column, C18 silica gel; mobile phase, MeCN in Water (10 mmol/L NH₄HCO₃), 10% to 1000% gradient in 60 min: detector. The purification yielded in 6-amino-3-ethyl-7-(3-methoxy-2,6-dimethylphenyl)imidazo[4,5-b]pyridine-5-carbonitrile (5 mg, 5.17%).

### Step 6: 6-Amino-3-ethyl-7-(3-methoxy-2,6-dimethylphenyl) imidazo[4,5-b]pyridine-5-carboxamide

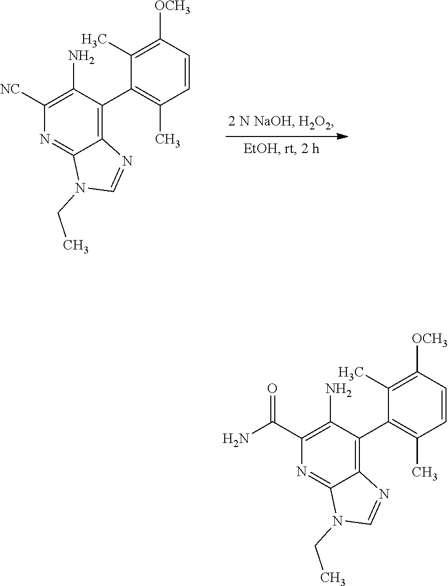 [SMILES: CCn1cnc2c(-c3c(C)ccc(OC)c3C)c(N)c(C#N)nc21 ; CCn1cnc2c(-c3c(C)ccc(OC)c3C)c(N)c(C(N)=O)nc21]

To a solution of 6-amino-3-ethyl-7-(3-methoxy-2,6-dimethylphenyl) imidazo[4,5-b]pyridine-5-carbonitrile (140 mg, 0.436 mmol, 1 equiv) in EtOH (2 mL) were added NaOH (17.42 mg, 0.436 mmol, 1 equiv) and H₂O₂ (128.45 mg, 1.132 mmol, 2 equiv, 30%). The mixture was stirred for 2 h at room temperature, and then extracted with EtOAc (3×30 mL). The combined organic layers were washed with water (3×30 mL) and dried over anhydrous Na₂SO₄. The residue was purified by preparative scale-TLC (PE/EA 1:1) to afford 6-amino-3-ethyl-7-(3-methoxy-2,6-dimethylphenyl) imidazo[4,5-b]pyridine-5-carboxamide (110 mg, 74.40%).

### Step 7: 6-Amino-3-ethyl-7-(3-hydroxy-2,6-dimethylphenyl)-3H-imidazo[4,5-b]pyridine-5-carboxamide

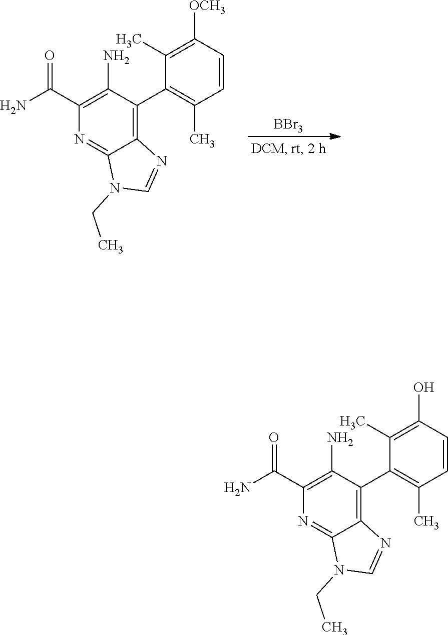

To a solution of 6-amino-3-ethyl-7-(3-methoxy-2,6-dimethylphenyl) imidazo[4,5-b]pyridine-5-carboxamide (20 mg, 0.059 mmol, 1 equiv) in DCM was added BBr₃ (294.64 uL, 0.295 mmol, 5 equiv) at 0° C. The mixture was stirred for 2 h at room temperature. The resulting mixture was diluted with MeOH (30 mL). The mixture was basified to pH 8 with NaHCO₃. The crude product was purified by Prep-HPLC to afford 6-amino-3-ethyl-7-(3-hydroxy-2,6-dimethylphenyl)-3H-imidazo[4,5-b]pyridine-5-carboxamide (4.8 mg, 3.72%). Column: Mobile Phase A: Water (10 mmol/L NH₄HCO₃+0.05% NH₃·H₂O), Mobile Phase B: ACN; Flow rate: 60 mL/min mL/min; Gradient: 17% B to 47% B in 8 min; detector wavelength: 254 nm.

LCMS (ES, m/z): [M+1]=326.10. ¹H NMR (300 MHz, Methanol-d4) δ 8.28 (s, 1H), 7.04 (d, J=8.2 Hz, 1H), 6.81 (d, J=8.2 Hz, 1H), 4.37 (q, J=7.3 Hz, 2H), 1.84 (d, J=9.0 Hz, 6H), 1.58 (t, J=7.3 Hz, 3H).

### Step 8: Chiral Separation

Crude 6-amino-3-ethyl-7-(3-hydroxy-2,6-dimethylphenyl)-3H-imidazo[4,5-b]pyridine-5-carboxamide (80 mg) product was purified by preparative-scale chiral HPLC using the following conditions: Column: Lux 5 μm Cellulose-4, 2.12×25 cm, 5 μm; Mobile Phase A: Hex (0.5% 2 M NH₃-MeOH)-HPLC, Mobile Phase B: EtOH-HPLC to afford (R)-6-amino-3-ethyl-7-(3-hydroxy-2,6-dimethylphenyl)-3H-imidazo[4,5-b]pyridine-5-carboxamide (18.9 mg)

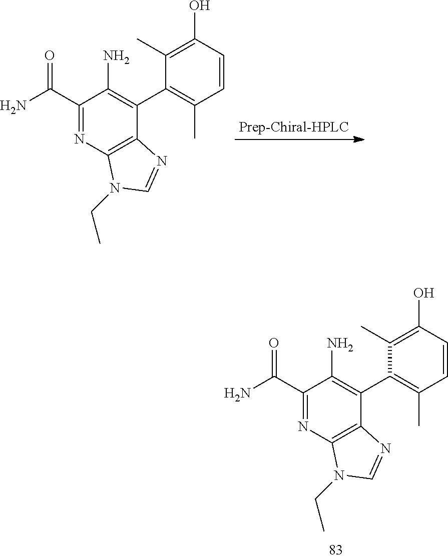

LCMS:(ES, m/z): [M+1]=326.10. ¹H NMR (300 MHz, DMSO-d₆) δ 9.18 (s, 1H), 8.35 (s, 1H), 8.16 (s, 1H), 7.45 (s, 1H), 6.98 (d, J=8.1 Hz, 1H), 6.80 (d, J=8.1 Hz, 1H), 5.80 (s, 2H), 4.27 (p, J=7.0 Hz, 2H), 1.76 (s, 3H), 1.70 (s, 3H), 1.48 (t, J=7.2 Hz, 3H).

### Example 70. (M)-5-amino-3-cyano-1-ethyl-4-(3-hydroxy-2,4-dimethylphenyl)-1H-pyrrolo[2,3-b]pyridine-6-carboxamide (82)

### Step 1: Methyl 5-bromo-1-ethylpyrrolo[2,3-b]pyridine-6-carboxylate

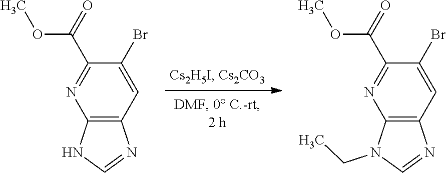 [SMILES: COC(=O)c1nc2[nH]cnc2cc1Br ; CCn1cnc2cc(Br)c(C(=O)OC)nc21]

A mixture of methyl 5-bromo-1H-pyrrolo[2,3-b]pyridine-6-carboxylate (1.50 g, 5.89 mmol, 1.00 equiv), ethyl iodide (1.10 mL, 7.06 mmol, 1.20 equiv) and Cs₂CO₃ (5.75 g, 17.64 mmol, 3.00 equiv) in DMF (30.00 mL) was stirred at room temperature for 2 h under nitrogen. The reaction mixture was quenched with H₂O (100.00 mL) and extracted with ethyl acetate (2×100.00 mL). The combined organic layers were washed with brine (2×100.00 mL), dried over anhydrous sodium sulfate, and concentrated under reduced pressure. The crude product was purified by column chromatography (column, silica gel; mobile phase, PE:EA=1:0 increasing to PE:EA=3:1 within 35 min; Detector. UV 254 nm), providing methyl 5-bromo-1-ethylpyrrolo[2,3-b]pyridine-6-carboxylate (1.66 g, 99.70%).

### Step 2: Methyl 5-bromo-3-cyano-1-ethylpyrrolo[2,3-b]pyridine-6-carboxylate

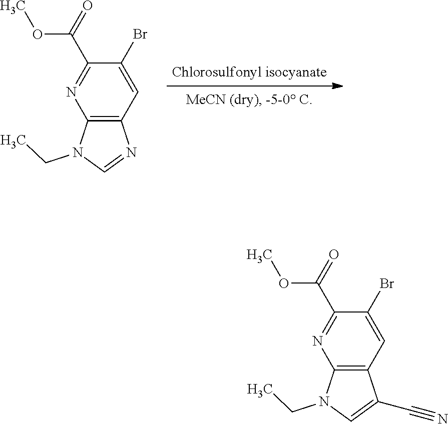 [SMILES: CCn1cnc2cc(Br)c(C(=O)OC)nc21 ; CCn1cc(C#N)c2cc(Br)c(C(=O)OC)nc21]

To a solution of methyl 5-bromo-1-ethylpyrrolo[2,3-b]pyridine-6-carboxylate (1.00 g, 3.53 mmol, 1.00 equiv) in dry acetonitrile (10.00 mL) in an ice bath maintained below −5° C. added was added a solution of chlorosulfonyl isocyanate (2.50 g, 17.66 mmol, 5.00 equiv) in 3.00 mL of dry acetonitrile dropwise over 2 min. Stirring was continued at 0° C. for 10 min. Dry N,N-dimethylformamide (2.00 ml) was then added, and the mixture was stirred at 0° C. for 30 min. The mixture was cooled to −40° C. using dry ice and quenched with ethyl acetate (30.00 mL). Addition of saturated NaHCO₃ resulted in solidification, so the mixture was frequently removed from the dry ice bath for swirling during the addition until the pH was 8-9. The reaction mixture was extracted with ethyl acetate (3×100.00 mL). The combined organic layers were washed with brine (2×100.00 mL), dried over anhydrous sodium sulfate, and concentrated under reduced pressure. The crude product was then purified by column chromatography (Column, silica gel: mobile phase, PE:EA=1:0 increasing to PE:EA=4:1 within 35 min; Detector, UV 254 nm), yielding methyl 5-bromo-3-cyano-1-ethylpyrrolo[2,3-b]pyridine-6-carboxylate (850.00 mg, 78.10%).

### Step 3: Methyl 3-cyano-5-[(diphenylmethylidene)amino]-1-ethylpyrrolo[2,3-b]pyridine-6-carboxylate

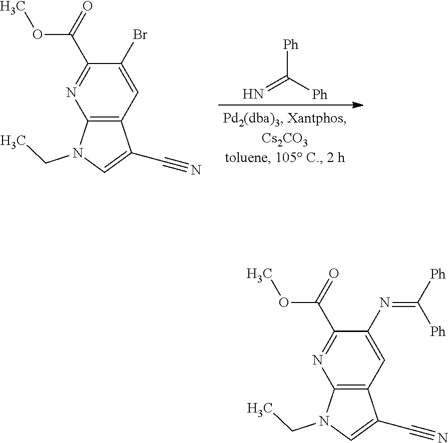 [SMILES: CCn1cc(C#N)c2cc(Br)c(C(=O)OC)nc21 ; N=C(c1ccccc1)c1ccccc1 ; CCn1cc(C#N)c2cc(N=C(c3ccccc3)c3ccccc3)c(C(=O)OC)nc21]

A mixture of methyl 5-bromo-3-cyano-1-ethylpyrrolo[2,3-b]pyridine-6-carboxylate (850.00 mg, 2.76 mmol, 1.00 equiv), α-phenyl-benzenemethanimine, (999.90 mg, 5.52 mmol, 2.00 equiv), Cs₂CO₃ (1797.56 mg, 5.52 mmol, 2.00 equiv), Pd₂(dba)₃ (252.61 mg, 0.28 mmol, 0.10 equiv) and xantphos (319.23 mg, 0.55 mmol, 0.20 equiv) in toluene (25.00 mL) was stirred at 105° C. for 2 h under nitrogen. The reaction mixture was cooled to room temperature, concentrated under reduced pressure, and purified by preparative scale TLC (PE:EA=2:1), affording methyl 3-cyano-5-[(diphenyl-methylidene)amino]-1-ethylpyrrolo[2,3-b]pyridine-6-carboxylate (743.00 mg, 65.94%).

### Step 4: Methyl 5-amino-3-cyano-1-ethylpyrrolo[2,3-b]pyridine-6-carboxylate

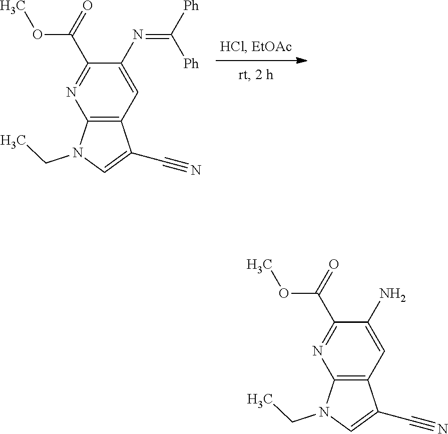 [SMILES: CCn1cc(C#N)c2cc(N=C(c3ccccc3)c3ccccc3)c(C(=O)OC)nc21 ; CCn1cc(C#N)c2cc(N)c(C(=O)OC)nc21]

To a solution of methyl 3-cyano-5-[(diphenylmethylidene)amino]-1-ethylpyrrolo[2,3-b]pyridine-6-carboxylate (740.00 mg, 1.81 mmol, 1.00 equiv) in EtOAc (80.00 mL) was added HCl (1.51 mL, 18.12 mmol, 10.00 equiv, 12M) at room temperature. The resulting solution was stirred for 2 h at room temperature. The reaction mixture was added with water (30.00 mL), was basified to pH 8-9 with saturated NaHCO₃ (aqueous) and extracted with ethyl acetate (3×50.00 mL). The combined organic layers were washed with brine (2×50.00 mL), dried over anhydrous sodium sulfate and concentrated under reduced pressure. Finally, the crude product was purified by preparative scale (PE:EA=2:1) to afford methyl 5-amino-3-cyano-1-ethylpyrrolo[2,3-b]pyridine-6-carboxylate (400.00 mg, 90.39%).

### Step 5: Methyl 5-amino-4-bromo-3-cyano-1-ethylpyrrolo[2,3-b]pyridine-6-carboxylate

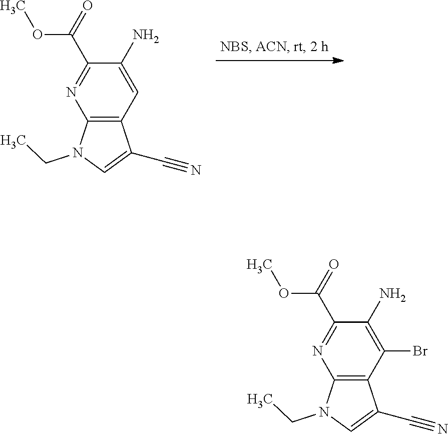 [SMILES: CCn1cc(C#N)c2cc(N)c(C(=O)OC)nc21 ; CCn1cc(C#N)c2c(Br)c(N)c(C(=O)OC)nc21]

A mixture of methyl 5-amino-3-cyano-1-ethylpyrrolo[2,3-b]pyridine-6-carboxylate (400.00 mg, 1.64 mmol, 1.00 equiv) and NBS (320.62 mg, 1.80 mmol, 1.10 equiv) in CH₃CN (20.00 mL) was stirred at room temperature for 2 h under nitrogen. H₂O (30.00 mL) was added, and the mixture was extracted with ethyl acetate (3×30.00 mL). The combined organic layers were washed with brine (2×30.00 mL), dried over anhydrous sodium sulfate, and concentrated under reduced pressure. Finally, the crude product was purified by preparative scale TLC (PE:EA=1:1) to afford methyl 5-amino-4-bromo-3-cyano-1-ethylpyrrolo[2,3-b]pyridine-6-carboxylate (516.00 mg, 97.51%).

### Step 6: Methyl 5-amino-3-cyano-1-ethyl-4-(3-methoxy-2,4-dimethylphenyl)pyrrolo[2,3-b]pyridine-6-carboxylate

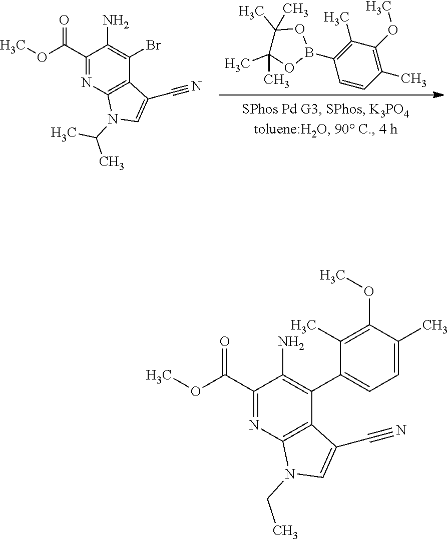

A mixture of methyl 5-amino-4-bromo-3-cyano-1-ethylpyrrolo[2,3-b]pyridine-6-carboxylate (500.00 mg, 1.55 mmol, 1.00 equiv), 2-(3-methoxy-2,4-dimethylphenyl)-4,4,5,5-tetramethyl-1,3,2-dioxaborolane (811.26 mg, 3.09 mmol, 2.00 equiv), K₃PO₄ (985.29 mg, 4.64 mmol, 3.00 equiv), SPhos (127.04 mg, 0.31 mmol, 0.20 equiv), SPhos Pd Gen.3 (120.73 mg, 0.16 mmol, 0.10 equiv) and H₂O (4.00 mL) in toluene (20.00 mL) was stirred at 90° C. for 4 h under nitrogen. The reaction mixture was cooled to room temperature, concentrated under reduced pressure, and purified by preparative scale TLC (PE:EA=2:1), providing methyl 5-amino-3-cyano-1-ethyl-4-(3-methoxy-2,4-dimethylphenyl)pyrrolo[2,3-b]pyridine-6-carboxylate (225.00 mg, 38.43%).

### Step 7: 5-Amino-3-cyano-1-ethyl-4-(3-methoxy-2,4-dimethylphenyl) pyrrolo[2,3-b]pyridine-6-carboxamide

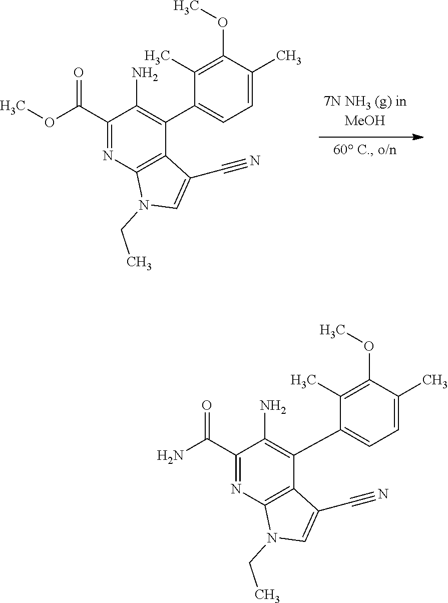

A mixture of methyl 5-amino-3-cyano-1-ethyl-4-(3-methoxy-2,4-dimethyl-phenyl)pyrrolo[2,3-b]pyri dine-6-carboxylate (100.00 mg, 0.26 mmol, 1.00 equiv) in NH₃(g) in MeOH (15.00 mL) was stirred overnight at 60° C. under nitrogen. The reaction mixture was cooled to room temperature and concentrated under reduced pressure. The crude product 5-amino-3-cyano-1-ethyl-4-(3-methoxy-2,4-dimethylphenyl) pyrrolo[2,3-b]pyridine-6-carboxamide (90.00 mg, 93.72%) was used in the next step directly without further purification.

### Step 8: 5-Amino-3-cyano-1-ethyl-4-(3-hydroxy-2,4-dimethylphenyl)pyrrolo[2,3-b]pyridine-6-carboxamide

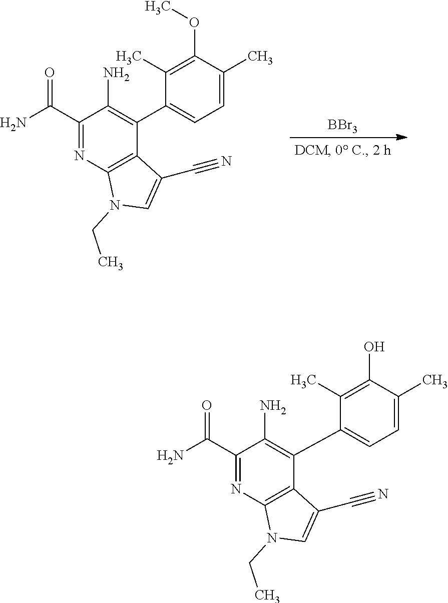 [SMILES: CCn1cc(C#N)c2c(-c3ccc(C)c(OC)c3C)c(N)c(C(N)=O)nc21 ; "BBr3" ; CCn1cc(C#N)c2c(-c3ccc(C)c(O)c3C)c(N)c(C(N)=O)nc21]

To a solution of 5-amino-3-cyano-1-ethyl-4-(3-methoxy-2,4-dimethyl-phenyl)pyrrolo[2,3-b]pyridine-6-carboxamide (90.00 mg, 0.25 mmol, 1.00 equiv) in methylene chloride (10.00 mL) was added BBr₃ (0.12 mL, 1.24 mmol, 5.00 equiv) at 0° C. The resulting solution was stirred for 2 h at 0° C. under nitrogen. The reaction mixture was quenched with ice water (30.00 mL), basified to pH 8-9 with saturated NaHCO₃ (aqueous) and extracted with methylene chloride (3×30.00 mL). The combined organic layers were washed with brine (2×30.00 mL), dried over anhydrous sodium sulfate, concentrated under reduced pressure, and purified by preparative scale TLC (DCM:MeOH=10:1). The product was further purified by preparative scale HPLC (Column: YMC-Actus Triart C18 ExRS 30×150 mm, 5 μm; Mobile Phase A: 10 mM NH₄HCO₃ (aqueous), Mobile Phase B: MeCN; flow rate: 60 mL/min; gradient: 30% B to 60% B in 9 min; detection wavelength: 254 nm/220 nm), resulting in 5-amino-3-cyano-1-ethyl-4-(3-hydroxy-2,4-dimethylphenyl)pyrrolo[2,3-b]pyridine-6-carboxamide (14.60 mg, 16.82%). LC-MS (ES, m/z): [M+1]=350.05. ¹H NMR (400 MHz, DMSO-d6) δ 8.45 (s, 1H), 8.38 (s, 1H), 8.27 (d, J=3.0 Hz, 1H), 7.51 (d, J=3.0 Hz, 1H), 7.06 (d, J=7.6 Hz, 1H), 6.57 (d, J=7.6 Hz, 1H), 5.93 (s, 2H), 4.43-4.27 (m, J=7.0 Hz, 2H), 2.25 (s, 3H), 1.86 (s, 3H), 1.43 (t, J=7.2 Hz, 3H).

### Step 9: Chiral Separation

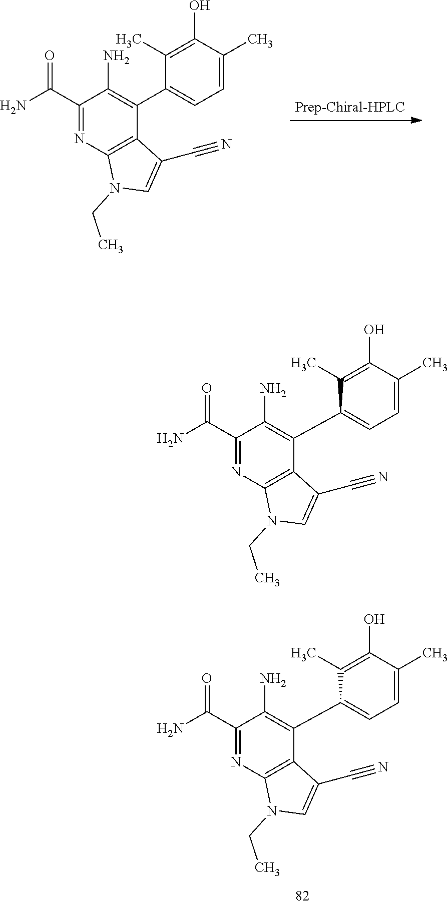

The product 5-amino-3-cyano-1-ethyl-4-(3-hydroxy-2,4-dimethylphenyl)-pyrrolo[2,3-b]pyridine-6-carb oxamide (50.00 mg, 0.14 mmol, 1.00 equiv) was purified by Prep-CHIRAL-HPLC with the following conditions: Column: CHIRALPAK IE, 2×25 cm, 5 μm; Mobile Phase A: hexane (0.5% 2M NH₃-MeOH), Mobile Phase B: EtOH; Flow rate: 20 mL/min; Gradient: isocratic 10; detection wavelength: 220/254 nm: RT1(min): 20.321; RT2(min): 24.52: Sample Solvent: EtOH; Injection Volume: 0.5 mL; Number Of Runs: 6.

The second eluting isomer was concentrated to afford (M)-5-amino-3-cyano-1-ethyl-4-(3-hydroxy-2,4-dimethylphenyl) pyrrolo[2,3-b]pyridine-6-carboxamide (82, 13.80 mg, 27.24%). (ES, m/z): [M+1]=350.05. ¹H NMR (300 MHz, DMSO-d6) δ 8.45 (s, 1H), 8.38 (s, 1H), 8.27 (d, J=3.0 Hz, 1H), 7.51 (d, J=2.9 Hz, 1H), 7.06 (d, J=7.6 Hz, 1H), 6.57 (d, J=7.6 Hz, 1H), 5.93 (s, 2H), 4.42-4.20 (m, 2H), 2.25 (s, 3H), 1.86 (s, 3H), 1.43 (t, J=7.2 Hz, 3H).

### Example 71. (P)-6-amino-7-(3-hydroxy-2,6-dimethylphenyl)-3-isopropyl-3H-imidazo[4,5-b]pyridine-5-carboxamide (83)

6-Amino-7-(3-hydroxy-2,6-dimethylphenyl)-3-isopropylimidazo-[4,5-b]pyridine-5-carboxamide was prepared in racemic form by the procedure described in Example 69, steps 3-7, using 3,5-diamino-6-(isopropylamino)pyridine-2-carbonitrile in place of 3,5-diamino-6-(ethylamino)pyridine-2-carbonitrile. The synthesis provided 6-amino-7-(3-hydroxy-2,6-dimethylphenyl)-3-isopropylimidazo[4,5-b]pyridine-5-carboxamide (9.20 mg, 31.84%), which was separated via chiral chromatography as described below. LCMS (ES, m/z): [M+1]=340.05. ¹H NMR (400 MHz, DMSO-d6) δ 9.21 (s, 1H), 8.42 (s, 1H), 8.15 (d, J=3.2 Hz, 1H), 7.47 (d, J=3.3 Hz, 1H), 6.99 (d, J=8.1 Hz, 1H), 6.81 (d, J=8.1 Hz, 1H), 5.81 (s, 2H), 4.99-4.90 (m, 1H), 1.73 (d, J=24.7 Hz, 6H), 1.56 (d, J=6.7 Hz, 6H).

The racemic material (40.00 mg, 0.11 mmol, 1.00 equiv. 90%) was purified by Prep-CHIRAL-HPLC with the following conditions: Column: CHIRAL ART Cellulose-SB 5×25 cm, 5 μm; Mobile Phase A: Hex(0.1% 2M NH₃-MeOH)-HPLC, Mobile Phase B: EtOH-HPLC; Gradient: isocratic SB 2 cm HEX(0.5% NH3·M): EtOH=90:10; Wave Length: 220/254 nm; RT1(min): 20.645; RT2(min): 28.116; Sample Solvent: EtOH-HPLC: Number Of Runs: 5.

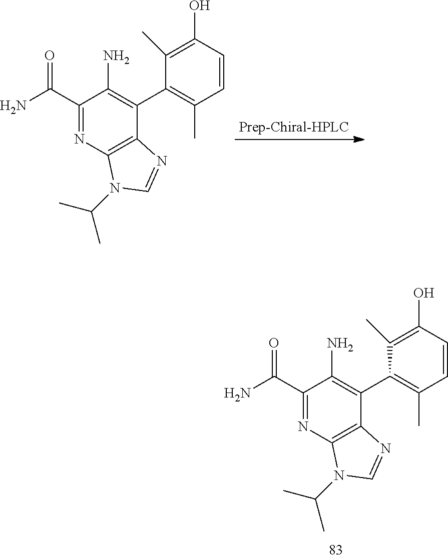

The second eluting isomer was concentrated to afford (6P)-6-amino-7-(3-hydroxy-2,6-dimethylphenyl)-3-isopropylimidazo[4,5-b]pyridine-5-carboxamide (83, 4.50 mg, 13.44%). LC-MS: (ES, m/z): [M+1]=340.05. ¹H NMR (400 MHz, DMSO-d₆) δ 9.21 (s, 1H), 8.42 (s, 1H), 8.15 (d, J=3.1 Hz, 1H), 7.46 (d, J=3.2 Hz, 1H), 6.99 (d, J=8.2 Hz, 1H), 6.81 (d, J=8.2 Hz, 1H), 5.80 (s, 2H), 4.95-4.89 (m, 1H), 1.73 (d, J=24.9 Hz, 6H), 1.57 (d, J=6.7 Hz, 6H).

### Example 72. (P)-6-amino-7-(3-hydroxy-2,6-dimethylphenyl)-3-isopropyl-2-methyl-3H-imidazo[4,5-b]pyridine-5-carboxamide (84)

6-Amino-7-(3-hydroxy-2,6-dimethylphenyl)-3-isopropyl-2-methylimidazo[4,5-b]pyridine-5-carboxamide was prepared in racemic form by the procedure described in Example 69, steps 3-7. In step 3, 3,5-diamino-6-(isopropylamino)pyridine-2-carbonitrile was used in place of 3,5-diamino-6-(ethylamino)pyridine-2-carbonitrile: trimethyl orthoacetate was used in place of trimethyl orthoformate; and acetic acid in place of hydrochloric acid. The synthesis provided 6-amino-7-(3-hydroxy-2,6-dimethylphenyl)-3-isopropyl-2-methylimidazo[4,5-b]pyridine-5-carboxamide (10.10 mg, 11.61%), which was separated via chiral chromatography as described below. LCMS (ES, m/z): [M+1]=354.05. ¹H NMR (400 MHz, DMSO-d6) δ 9.21 (s, 1H), 7.91 (d, J=3.3 Hz, 1H), 7.42 (d, J=3.3 Hz, 1H), 6.97 (d, J=8.1 Hz, 1H), 6.80 (d, J=8.1 Hz, 1H), 5.77 (s, 2H), 4.90-4.75 (m, 1H), 2.49 (s, 3H), 1.76 (s, 3H), 1.70 (s, 3H), 1.64 (d, J=6.8 Hz, 6H).

The product 6-amino-7-(3-hydroxy-2,6-dimethylphenyl)-3-isopropyl-2-methylimidazo[4,5-b]pyridine-5-carboxamide (50.00 mg, 0.14 mmol, 1.00 equiv) was purified by Prep-CHIRAL-HPLC with the following conditions: Column: CHIRALPAK ID, 2*25 cm, 5 μm; Mobile Phase A: MTBE(0.5% 2M NH₃-MeOH)-HPLC, Mobile Phase B: EtOH-HPLC; Flow rate: 20 mL/min: Gradient: isocratic 5; Wave Length: 220/254 nm; RT1(min): 5.89; RT2(min): 7.26; Sample Solvent: EtOH-HPLC: Injection Volume: 0.5 mL; Number Of Runs: 14.

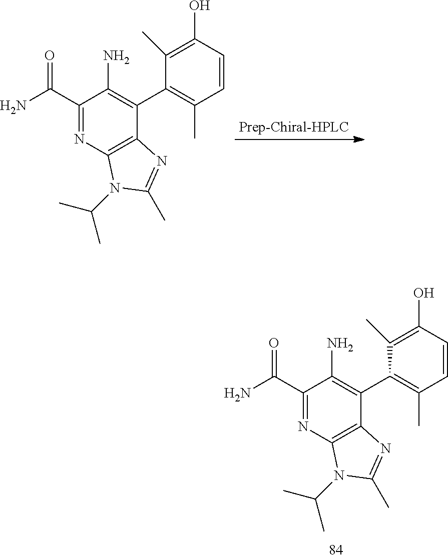

The second eluting isomer was concentrated to afford (P)-6-amino-7-(3-hydroxy-2,6-dimethylphenyl)-3-isopropyl-2-methylimidazo[4,5-b]pyridine-5-carboxamide (84, 10.20 mg, 20.30%). LC-MS (ES, m/z): [M+1]=354.05. ¹H NMR (400 MHz, DMSO-d₆) δ 9.19 (s, 1H), 7.92 (d, J=3.3 Hz, 1H), 7.42 (d, J=3.3 Hz, 1H), 6.97 (d, J=8.2 Hz, 1H), 6.80 (d, J=8.2 Hz, 1H), 5.77 (s, 2H), 4.85-4.74 (m, 1H), 2.49 (s, 3H), 1.73 (d, J=24.7 Hz, 6H) 1.64 (d, J=6.8 Hz, 6H).

### Example 73. (P)-5-amino-1-(difluoromethyl)-4-(3-hydroxy-2,6-dimethylphenyl)-1H-benzo[d]imidazole-6-carboxamide (85)

### Step 1: 4-Amino-2-fluoro-5-nitro-benzonitrile

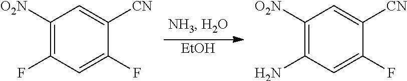 [SMILES: "EtOH" ; N#Cc1cc([N+](=O)[O-])c(F)cc1F ; N#Cc1cc([N+](=O)[O-])c(N)cc1F]

To a solution of 2,4-difluoro-5-nitro-benzonitrile (15 g, 81.48 mmol, 1 eq) in EtOH (100 mL) was added NH₃·H₂O (12.5 mL, 28% purity, 1 eq) at 0° C. After stirring at 25° C. for 16 hours, the reaction mixture was concentrated directly to afford 4-amino-2-fluoro-5-nitro-benzonitrile (72 g, crude). ¹H NMR: (400 MHz, DMSO-d₆) δ=8.60 (s, 1H), 8.14 (bs, 2H), 6.92 (d, 1H).

### Step 2: 4-Amino-3-bromo-2-fluoro-5-nitro-benzonitrile

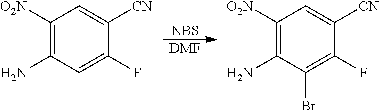

To a solution of 4-amino-2-fluoro-5-nitro-benzonitrile (18 g, 99.38 mmol, 1 eq. 4 batches) in DMF (150 mL) was added NBS (26.5 g, 149.07 mmol, 1.5 eq) at 0° C. The mixture was stirred at 30° C. for 16 hours. The mixture was then diluted with water (200 mL) and filtered directly to give a filter cake. The crude product was triturated with EtOH/120 (1:3, 100 mL), filtered, and concentrated to provide 4-amino-3-bromo-2-fluoro-5-nitro-benzonitrile (84 g, crude). ¹H NMR: (400 MHz, DMSO-d₆) δ=8.73 (d, 1H), 8.12 (s, 2H).

### Step 3: 4-Amino-2-fluoro-3-(3-methoxy-2,6-dimethyl-phenyl)-5-nitro-benzonitrile

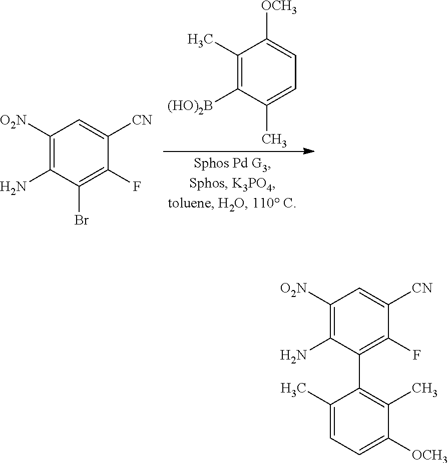 [SMILES: N#Cc1cc([N+](=O)[O-])c(N)c(Br)c1F ; COc1ccc(C)c(B(O)O)c1C ; COc1ccc(C)c(-c2c(N)c([N+](=O)[O-])cc(C#N)c2F)c1C]

To a solution of 4-amino-3-bromo-2-fluoro-5-nitro-benzonitrile (10 g, 38.46 mmol, 1 eq, 3 batches) and (3-methoxy-2,6-dimethyl-phenyl)boronic acid (13.9 g, 76.92 mmol, 2 eq) in toluene (120 mL) and H₂O (40 mL) were added SPhos Pd G₃ (6.0 g, 7.69 mmol, 0.2 eq), SPhos (6.3 g, 15.38 mmol, 0.4 eq), and K₃PO₄ (40.8 g, 192.29 mmol, 5 eq). The mixture was degassed with N₂ and stirred at 110° C. for 36 hours. The mixture was diluted with water (100 mL) and extracted with EtOAc (100 mL×3). The combined organic layers were washed with aqueous saturated NaCl (100 mL×2), dried over anhydrous Na₂SO₄, filtered, and concentrated to provide a residue. The residue was purified by flash silica gel chromatography (ISCO; 330 g SEPAFLASH Silica Flash Column, eluent of 0-10% ethyl acetate/petroleum ether gradient at 100 mL/min) to afford 4-amino-2-fluoro-3-(3-methoxy-2,6-dimethyl-phenyl)-5-nitro-benzonitrile (13.3 g, crude). ¹H NMR: (400 MHz, DMSO-d₆) δ=8.77 (d, 1H), 7.23 (d, 1H), 7.04 (d, 1H), 3.82 (s, 3H), 1.93 (s, 3H), 1.86 (s, 3H).

### Step 4: 4-Amino-3-(3-methoxy-2,6-dimethyl-phenyl)-2-[(4-methoxyphenyl)-methylamino]-5-nitro-benzonitrile

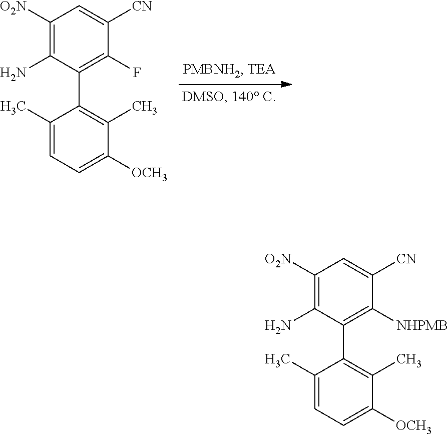

To a solution of 4-amino-2-fluoro-3-(3-methoxy-2,6-dimethyl-phenyl)-5-nitro-benzonitrile (13.3 g, 42.18 mmol, 1 eq) and (4-methoxyphenyl)methanamine (11.6 g, 84.36 mmol, 11 mL, 2 eq) in DMSO (70 mL) was added TEA (8.5 g, 84.36 mmol, 12 mL, 2 eq). After stirring at 140° C. for 3 hours, the mixture was adjusted to pH around 6 with aqueous HCl (2 M), diluted with H₂O (50 mL) and filtered directly to give 4-amino-3-(3-methoxy-2,6-dimethyl-phenyl)-2-[(4-methoxyphenyl)methylamino]-5-nitro-benzonitrile (21 g, crude). ¹H NMR: (400 MHz, DMSO-d₆) δ=8.35 (s, 1H), 7.23 (s, 1H), 7.08-6.99 (m, 3H), 6.84 (d, 2H), 5.70 (t, 1H), 4.54 (d, 2H), 3.81 (s, 3H), 3.70 (s, 3H), 1.83 (s, 3H), 1.78 (s, 3H).

### Step 5: 4,5-Diamino-3-(3-methoxy-2,6-dimethyl-phenyl)-2-[(4-methoxyphenyl)-methylamino]benzonitrile

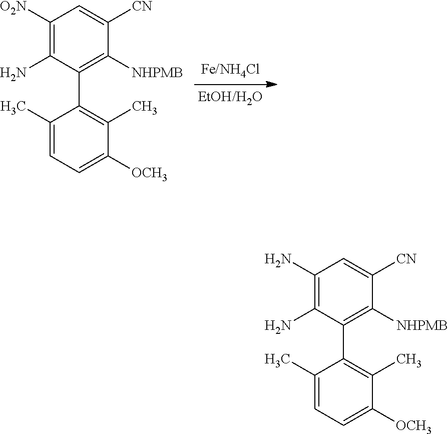

To a solution of 4-amino-3-(3-methoxy-2,6-dimethyl-phenyl)-2-[(4-methoxyphenyl)methylamino]-5-nitro-benzonitrile (10.5 g, 24.28 mmol, 1 eq) in EtOH (80 mL) and H₂O (80 mL) were added Fe (6.8 g, 121.40 mmol, 5 eq) and NH₄C₁ (6.5 g, 121.40 mmol, 5 eq). The mixture was stirred at 85° C. for 2 hours. The mixture was then filtered, and the filtrate was diluted with water (200 mL) and extracted with EtOAc (200 mL×3). The combined organic layers were washed with aqueous saturated NaCl (100 mL×2), dried over anhydrous Na₂SO₄, filtered, and concentrated to give a residue which was purified by flash silica gel chromatography (ISCO; 330 g SEPAFLASH Silica Flash Column, eluent of 0~50% ethyl acetate/petroleum ether, gradient at 100 mL/min), affording 4,5-diamino-3-(3-methoxy-2,6-dimethyl-phenyl)-2-[(4-methoxyphenyl)methylamino]benzonitrile (12.9 g, 28.85 mmol, 90% purity). LCMS: m/z 425.1 [M+Na]⁺

### Step 6: 7-(3-Methoxy-2,6-dimethyl-phenyl)-6-[(4-methoxyphenyl)methylamino]-3H-benzimidazole-5-carbonitrile

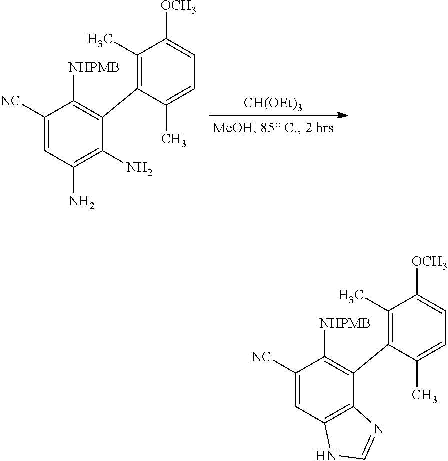

To a solution of 4,5-diamino-3-(3-methoxy-2,6-dimethyl-phenyl)-2-[(4-methoxyphenyl) methylamino]benzonitrile (700 mg, 1.74 mmol, 1 eq) in MeOH (10 mL) was added triethylorthoformate (387 mg, 2.61 mmol, 1.5 eq). The mixture was stirred at 85° C. for 2 hours and concentrated to give a residue, which was purified by flash silica gel chromatography (ISCO; 12 g SEPAFLASH Silica Flash Column, eluent of 0~80% ethyl acetate/petroleum ether gradient at 30 mL/min), affording 7-(3-methoxy-2,6-dimethyl-phenyl)-6-[(4-methoxyphenyl)methylamino]-3H-benzimidazole-5-carbonitrile (597 mg, 1.29 mmol, 74% yield, 89% purity). LCMS: m/z 413.1 [M+H]⁺

### Step 7: 3-(Difluoromethyl)-7-(3-methoxy-2,6-dimethyl-phenyl)-6-[(4-methoxyphenyl)methylamino]benzimidazole-5-carbonitrile

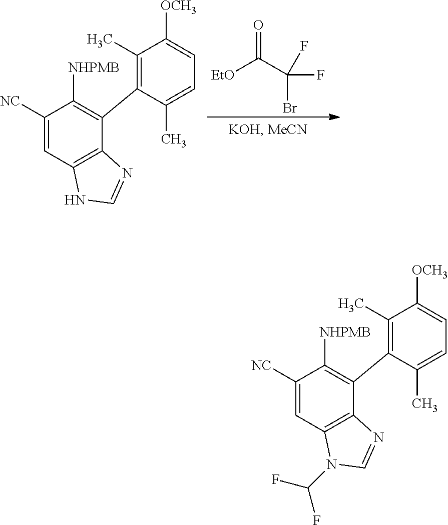

To a solution of 7-(3-methoxy-2,6-dimethyl-phenyl)-6-[(4-methoxyphenyl)methylamino]-3H-benzimidazole-5-carbonitrile (600 mg, 1.45 mmol, 1 eq), ethyl 2-bromo-2,2-difluoro-acetate (443 mg, 2.18 mmol, 0.3 mL, 1.5 eq) in MeCN (7 mL) was added KOH (82 mg, 1.45 mmol, 1 eq). The mixture was stirred at 60° C. for 12 hours under N₂. The reaction mixture was then diluted with H₂O (100 mL) and extracted with EtOAc (50 mL×4). The combined organic layers were washed with aqueous saturated NaCl (50 mL×2), dried over anhydrous Na₂SO₄, filtered, and concentrated under vacuum. The resulting crude product was purified by flash silica gel chromatography (ISCO; 12 g SEPAFLASH Silica Flash Column, eluent of 0~80% EtOAc/PE at 30 mL/min) to give 3-(difluoromethyl)-7-(3-methoxy-2,6-dimethyl-phenyl)-6-[(4-methoxyphenyl)methylamino]-benzimidazole-5-carbonitrile (600 mg, 0.87 mmol, 67.2% purity). LCMS: m/z 463.3 [M+H]⁺.

### Step 8: 3-(Difluoromethyl)-7-(3-methoxy-2,6-dimethyl-phenyl)-6-[(4-methoxyphenyl) methylamino]benzimidazole-5-carbonitrile

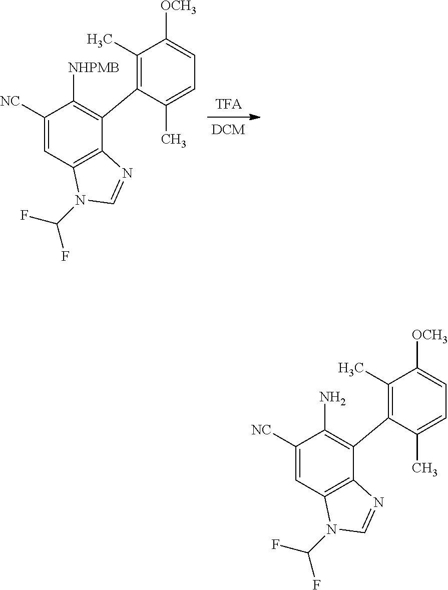

To a solution of 3-(difluoromethyl)-7-(3-methoxy-2,6-dimethyl-phenyl)-6-[(4-methoxyphenyl) methylamino]benzimidazole-5-carbonitrile (600 mg, 1.30 mmol, 1 eq) in DCM (2 mL) was added TFA (1 mL). The mixture was stirred at 15° C. for 2 hours, then was adjusted to a pH around 7 with saturated NaHCO₃ and extracted with DCM 150 mL (50 mL×3). The combined organic layers were washed with aqueous saturated NaCl (50 mL×2), dried over anhydrous Na₂SO₄, filtered, and concentrated to afford 6-amino-3-(difluoromethyl)-7-(3-methoxy-2,6-dimethyl-phenyl)benzimidazole-5-carbonitrile (177 mg, crude). LCMS: m/z 343.1 [M+H]⁺.

### Step 9: 6-Amino-3-(difluoromethyl)-7-(3-methoxy-2,6-dimethyl-phenyl)benzimidazole-5-carboxamide

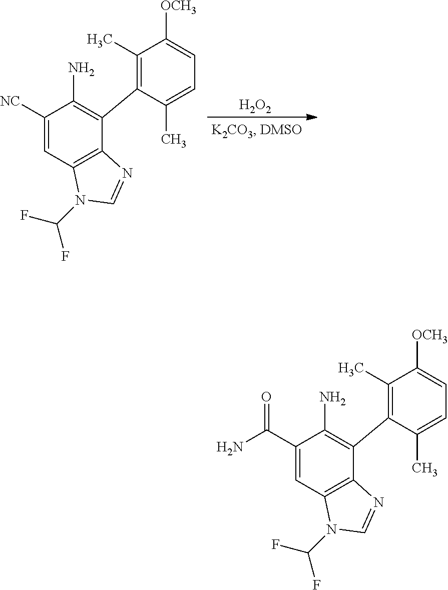

To a solution of 6-amino-3-(difluoromethyl)-7-(3-methoxy-2,6-dimethyl-phenyl)benzimidazole-5-carbonitrile (578 mg, 1.69 mmol, 1 eq) in DMSO (6 mL) was added H₂O₂ (1.91 g, 16.88 mmol, 30% purity, 10 eq) and K₂CO₃ (467 mg, 3.38 mmol, 2 eq). After stirring at 25° C. for 12 hours, the mixture was diluted with H₂O (20 mL) at 0° C. and filtered to give a filter cake. The filter cake was further purified by flash silica gel chromatography (ISCO; 12 g SEPAFLASH Silica Flash Column, Eluent of 0~10% MeOH/DCM qt 30 mL/min) to provide 6-amino-3-(difluoromethyl)-7-(3-methoxy-2,6-dimethyl-phenyl)benzimidazole-5-carboxamide (430 mg, 0.96 mmol, 80% purity). LCMS: m/z 361.0 [M+H]⁺.

### Step 10: 6-Amino-3-(difluoromethyl)-7-(3-hydroxy-2,6-dimethyl-phenyl)benzimidazole-5-carboxamide

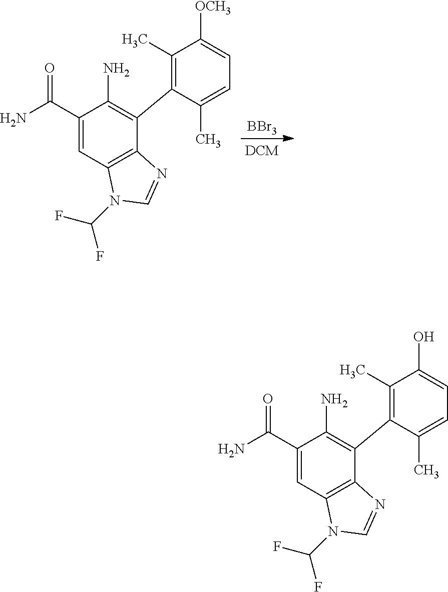

To a solution of 6-amino-3-(difluoromethyl)-7-(3-methoxy-2,6-dimethyl-phenyl)benzimidazole-5-carboxamide (200 mg, 0.55 mmol, 1 eq) in DCM (2 mL) was added BBr₃ (0.3 mL, 2.78 mmol, 5 eq) at 0° C. The mixture was stirred at 15° C. for 2 hours. The reaction mixture was quenched by water (10 mL), then adjusted to pH=8 with aqueous saturated NaHCO₃ at 0° C., followed by extracted with DCM (10 mL×3). The combined organic layers were washed with aqueous saturated NaCl (10 mL×2) dried over anhydrous Na₂SO₄, filtered and concentrated under vacuum. The crude product was purified by prep-HPLC (column: Xtimate C18 150×40 mm×10 um; mobile phase: [water(FA)-ACN]; gradient:0%-36% B over 36 min) to give 6-amino-3-(difluoromethyl)-7-(3-hydroxy-2,6-dimethyl-phenyl)benzimidazole-5-carboxamide (35 mg, 0.10 mmol, 18% yield, 100.00% purity). LCMS: m/z 347.1 [M+H]⁺. ¹H NMR: (400 MHz, DMSO-d6) δ=9.10 (s, 1H) 8.38 (s, 1H) 7.74-8.11 (m, 3H) 7.23-7.40 (m, 1H) 6.96 (d, 1H) 6.77 (d, 1H) 5.29-5.56 (m, 2H) 1.75 (s, 3H) 1.69 (s, 3H).

### Step 11: Chiral Separation

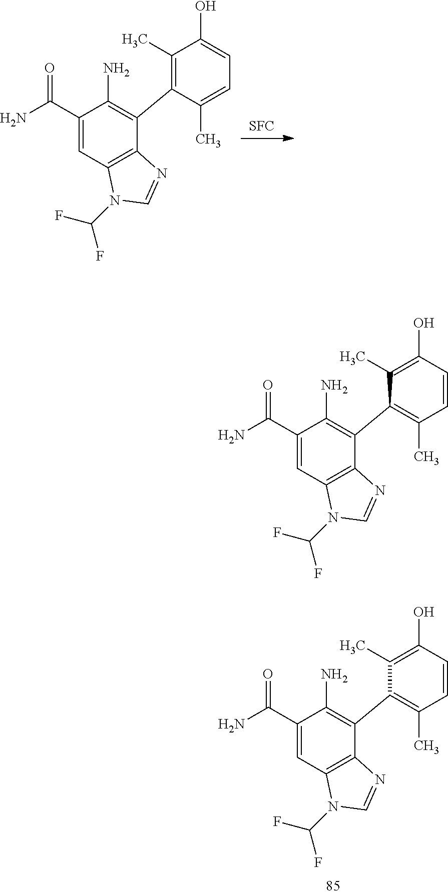

The racemate of 6-amino-3-(difluoromethyl)-7-(3-hydroxy-2,6-dimethyl-phenyl)benzimidazole-5-carboxamide (60 mg, 0.17 mmol, 1 eq) was separated by SFC separation (column: DAICEL CHIRALCEL OD (250 mm×30 mm, 10 um); mobile phase: [CO₂-EtOH]:B %:45%, isocratic elution mode) to give (P)-6-amino-3-(difluoromethyl)-7-(3-hydroxy-2,6-dimethyl-phenyl)benzimidazole-5-carboxamide (85, 8.1 mg, 0.023 mmol, 97.59% purity).

LCMS: m/z 347.1 [M+H]⁺. ¹H NMR: (400 MHz, DMSO-d₆) δ=9.09 (s, 1H) 8.37 (s, 1H) 7.78-8.10 (m, 3H) 7.16-7.44 (m, 1H) 6.96 (d, 1H) 6.77 (d, 1H) 5.41 (s, 2H) 1.75 (s, 3H) 1.69 (s, 3H).

### Example 74. (P)-6-amino-7-(3-hydroxy-2,6-dimethylphenyl)-3-(2,2,2-trifluoroethyl)-3H-imidazo[4,5-b]pyridine-5-carboxamide (86)

### Step 1: 6-Amino-3H-imidazo[4,5-b]pyridine-5-carbonitrile

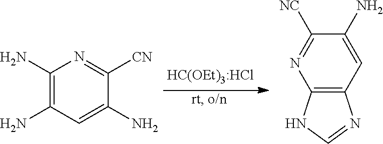 [SMILES: N#Cc1nc(N)c(N)cc1N ; "rt, o/n" ; N#Cc1nc2[nH]cnc2cc1N]

A mixture of 3,5,6-triaminopyridine-2-carbonitrile (3 g, 20.113 mmol, 1 equiv), HC(OEt)₃ (120 mL) and HCl (3 mL) was stirred overnight at room temperature. The mixture was then basified to pH 8 with saturated NaHCO₃(aqueous), and the aqueous layer was extracted with EtOAc. The residue was purified by silica gel column chromatography, eluting with CH₂Cl₂/MeOH (20:1) to afford 6-amino-3H-imidazo[4,5-b]pyridine-5-carbonitrile (1.7 g, 53.11%).

### Step 2: N-(5-Cyano-3H-imidazo[4,5-b]pyridin-6-yl-2,2,2-trifluoroacetamide

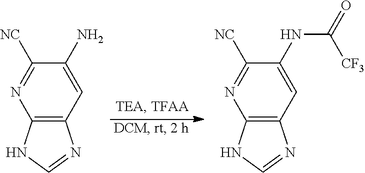 [SMILES: N#Cc1nc2[nH]cnc2cc1N ; "DCM, rt, 2 h" ; N#Cc1nc2[nH]cnc2cc1NC(=O)C(F)(F)F]

To a stirred solution of 6-amino-3H-imidazo[4,5-b]pyridine-5-carbonitrile (1.7 g, 10.682 mmol, 1 equiv) in DCM (30 mL) were added TEA (7.4 mL, 53.237 mmol, 5.00 equiv) and TFAA (6 mL, 43.136 mmol, 4.00 equiv) in portions at 0° C. The mixture was stirred for 2 h at room temperature before concentrating under reduced pressure. The resulting residue was purified by silica gel column chromatography, eluting with PE/EA (1:2) to afford N-(5-cyano-3H-imidazo[4,5-b]pyridin-6-yl-2,2,2-trifluoroacetamide (2.6 g, 9539%).

### Step 3: N-[5-Cyano-3-(2,2,2-trifluoroethyl)imidazo[4,5-b]pyridin-6-yl]-2,2,2-trifluoroacetamide

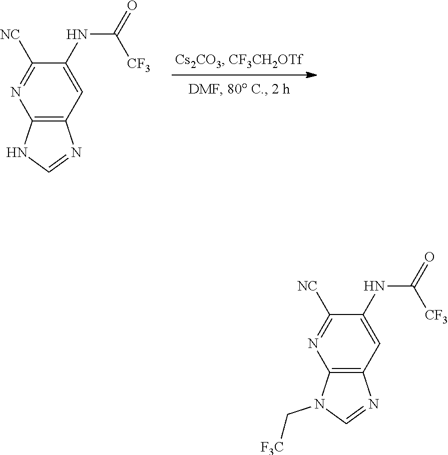 [SMILES: N#Cc1nc2[nH]cnc2cc1NC(=O)C(F)(F)F ; N#Cc1nc2c(cc1NC(=O)C(F)(F)F)ncn2CC(F)(F)F]

To a stirred solution of N-(5-cyano-3H-imidazo[4,5-b]pyridin-6-yl-2,2,2-trifluoroacetamide (2.6 g, 10.190 mmol, 1 equiv) in DMF (30 mL) were added Cs₂CO₃ (10 g, 30.692 mmol, 3.00 equiv) and CF₃CH₂OTf (7.3 mL) in portions at 0° C. The mixture was stirred for 2 h at 80° C. before concentrating under reduced pressure. The resulting residue was purified by silica gel column chromatography (PE/EA 1:2) to afford N-[5-cyano-3-(2,2,2-trifluoroethyl)imidazo[4,5-b]pyridin-6-yl]-2,2,2-trifluoroacetamide (1.3 g, 37.84%).

### Step 4: 6-Amino-3-(2,2,2-trifluoroethyl)imidazo[4,5-b]pyridine-5-carbonitrile

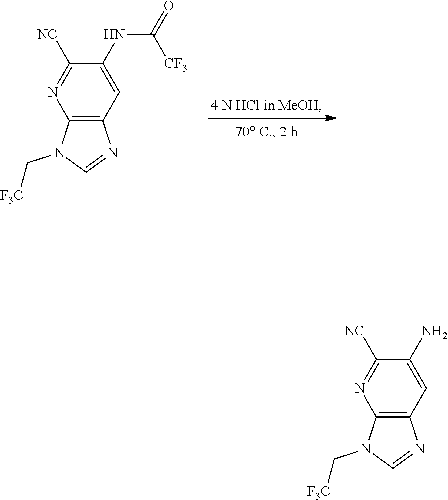 [SMILES: N#Cc1nc2c(cc1NC(=O)C(F)(F)F)ncn2CC(F)(F)F ; N#Cc1nc2c(cc1N)ncn2CC(F)(F)F]

A mixture of N-[5-cyano-3-(2,2,2-trifluoroethyl)imidazo[4,5-b]pyridin-6-yl]-2,2,2-trifluoroacetamide (1.3 g, 3.855 mmol, 1 equiv) and 4 N HCl(g) in MeOH (28 mL) was stirred for 2 h at 70° C. The mixture was concentrated under reduced pressure, and the resulting residue was purified by silica gel column chromatography using PE/EA (1:2) to afford 6-amino-3-(2,2,2-trifluoroethyl)imidazo[4,5-b]pyridine-5-carbonitrile (0.8 g, 88.89%).

### Step 5: 6-Amino-7-bromo-3-(2,2,2-trifluoroethyl)imidazo[4,5-b]pyridine-5-carbonitrile

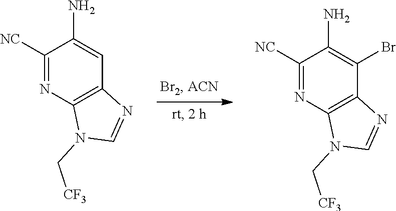 [SMILES: N#Cc1nc2c(cc1N)ncn2CC(F)(F)F ; N#Cc1nc2c(ncn2CC(F)(F)F)c(Br)c1N]

To a stirred solution of 6-amino-3-(2,2,2-trifluoroethyl)imidazo[4,5-b]pyridine-5-carbonitrile (150 mg, 0.6 12 mmol, 1 equiv) in acetonitrile (10 mL) was added Br₂ (150 mg, 0.923 mmol, 1.50 equiv) dropwise at 0° C. The resulting mixture was stirred for 2 h at room temperature. The reaction was quenched with MeOH at 0° C., and the mixture was concentrated under reduced pressure. The resulting residue was purified by silica gel column chromatography, eluting with CH₂Cl₂/MeOH (20:1) to afford 6-amino-7-bromo-3-(2,2,2-trifluoroethyl)imidazo[4,5-b]pyridine-5-carbonitrile (150 mg, 75.75%).

### Step 6: 6-Amino-7-(3-methoxy-2,6-dimethylphenyl)-3-(2,2,2-trifluoroethyl)imidazo[4,5-b]pyridine-5-carbonitrile

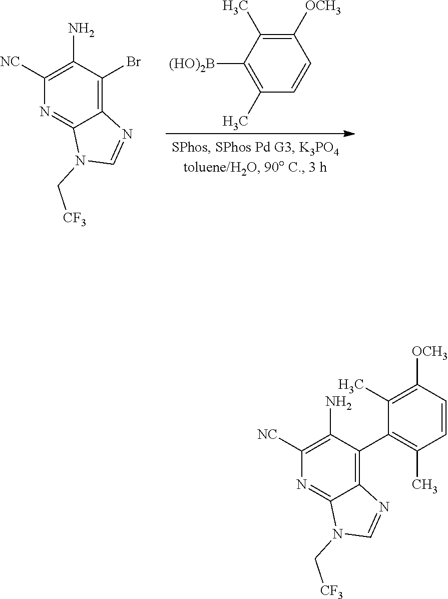 [SMILES: N#Cc1nc2c(ncn2CC(F)(F)F)c(Br)c1N ; COc1ccc(C)c(B(O)O)c1C ; COc1ccc(C)c(-c2c(N)c(C#N)nc3c2ncn3CC(F)(F)F)c1C]

To a stirred solution of 6-amino-7-bromo-3-(2,2,2-trifluoroethyl)imidazo[4,5-b]pyridine-5-carbonitrile (500 mg, 1.562 mmol, 1 equiv) and 3-methoxy-2,6-dimethylphenylboronic acid (337.44 mg, 1.874 mmol, 1.2 equiv) in toluene (60 mL) and H₂O (12 mL) were added K₃PO₄ (663.18 mg, 3.124 mmol, 2 equiv), SPhos (256.53 mg, 0.625 mmol, 0.4 equiv) and SPhos Pd Gen.3 (243.78 mg, 0.312 mmol, 0.2 equiv) in portions at room temperature under nitrogen. The resulting mixture was stirred for 3 h at 90° C. under nitrogen before extraction with EtOAc (3×50 mL). The combined organic layers were dried over anhydrous Na₂SO₄ and concentrated under reduced pressure. The resulting residue was purified by silica gel column chromatography (PE/EA 1:2) to afford 6-amino-7-(3-methoxy-2,6-dimethylphenyl)-3-(2,2,2-trifluoroethyl)imidazo[4,5-b]pyridine-5-carbonitrile (117 mg, 19.95%).

### Step 7: 6-Amino-7-(3-methoxy-2,6-dimethylphenyl)-3-(2,2,2-trifluoroethyl)imidazo[4,5-b]pyridine-5-carboxamide

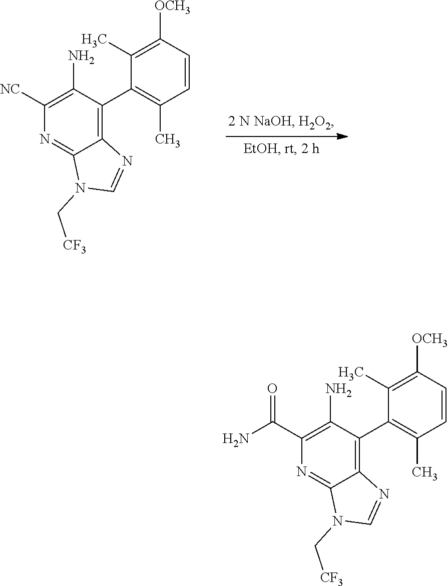 [SMILES: COc1ccc(C)c(-c2c(N)c(C#N)nc3c2ncn3CC(F)(F)F)c1C ; COc1ccc(C)c(-c2c(N)c(C(N)=O)nc3c2ncn3CC(F)(F)F)c1C]

A mixture of 6-amino-7-(3-methoxy-2,6-dimethylphenyl)-3-(2,2,2-trifluoroethyl)imidazo[4,5-b]pyridine-5-carbonitrile (134 mg, 0.357 mmol, 1 equiv), NaOH (358 μL, 1 equiv, 2 N) and H₂O₂ (180 μL) in EtOH (5 mL) was stirred for 2 h at room temperature. The mixture was then concentrated under reduced pressure, and the resulting residue was purified by silica gel column chromatography, eluting with PE/EA (1:1) to afford 6-amino-7-(3-methoxy-2,6-dimethylphenyl)-3-(2,2,2-trifluoroethyl)imidazo[4,5-b]pyridine-5-carboxamide (107 mg, 76.19%).

### Step 8. 6-Amino-7-(3-hydroxy-2,6-dimethylphenyl)-3-(2,2,2-trifluoroethyl)imidazo[4,5-b]pyridine-5-carboxamide

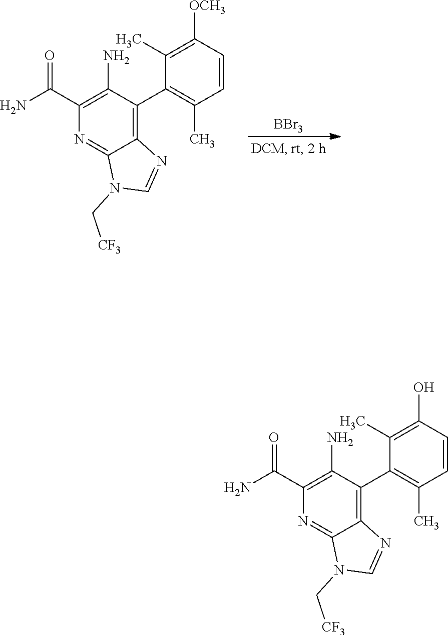

To a stirred solution of 6-amino-7-(3-methoxy-2,6-dimethylphenyl)-3-(2,2,2-trifluoroethyl)imidazo[4,5-b]pyridine-5-carboxamide (105 mg, 0.267 mmol, 1 equiv) in DCM (3 mL) was added BBr₃ (1.87 mL, 5 equiv) dropwise at room temperature. The resulting mixture was stirred for 2 h at room temperature. The reaction was quenched with MeOH at 0° C. The mixture was basified with saturated NaHCO₃ (aqueous) and filtered, and the filter cake was washed with MeOH. The filtrate was concentrated under reduced pressure. The crude product was purified by preparative-scale HPLC to afford 6-amino-7-(3-hydroxy-2,6-dimethylphenyl)-3-(2,2,2-trifluoroethyl)imidazo[4,5-b]pyridine-5-carboxamide (11.2 mg, 11.03%). Mobile phase, water (10 mmol/L NH₄HCO₃+0.1% NH₃·H₂O) and ACN (5% ACN up to 35% in 9 min). LCMS (ES, m/z): [M+1]=380.10. ¹H NMR (300 MHz, DMSO-d6) δ 9.23 (s, 1H), 8.41 (d, J=9.1 Hz, 2H), 7.52 (d, J=3.2 Hz, 1H), 7.00 (d, J=8.2 Hz, 1H), 6.82 (d, J=8.2 Hz, 1H), 5.93 (s, 2H), 5.30 (q, J=9.4 Hz, 2H), 1.70-1.77 (s, 6H).

### Step 9. Chiral Separation

The racemic material was purified by preparative-scale chiral HPLC with the following conditions: mobile phase, Hex (0.1% DEA) and IPA (hold 20% IPA). Column: CHIRALPAK ID, 2*25 cm, 5 μm.

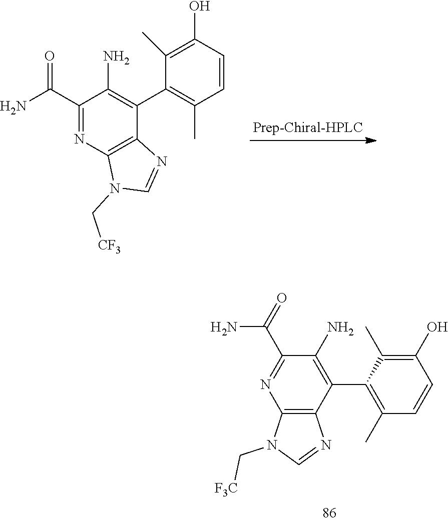

The chiral separation yielded (P)-6-amino-7-[(1R)-5-hydroxy-2,6-dimethylcyclohexa-2,4-dien-1-yl]-3-(2,2,2-trifluoroethyl)imidazo[4,5-b]pyridine-5-carboxamide (3.8 mg, 12.60%). LCMS (ES, m/z): [M+1]=380.10. ¹H NMR (300 MHz, Methanol-d4) δ 8.35 (s, 1H), 7.05 (d, J=8.2 Hz, 1H), 6.83 (d, J=8.2 Hz, 1H), 5.18 (q, J=9.0 Hz, 2H), 1.85 (d, J=8.6 Hz, 6H).

### Example 75. (P)-5-amino-1-(3-fluoropyridin-2-yl)-4-(3-hydroxy-2,6-dimethylphenyl)-1H-benzo[d]imidazole-6-carboxamide (87)

### Step 1: 7-(3-Benzyloxy-2,6-dimethyl-phenyl)-6-[(4-methoxyphenyl)methylamino]-3H-benzimidazole-5-carbonitrile

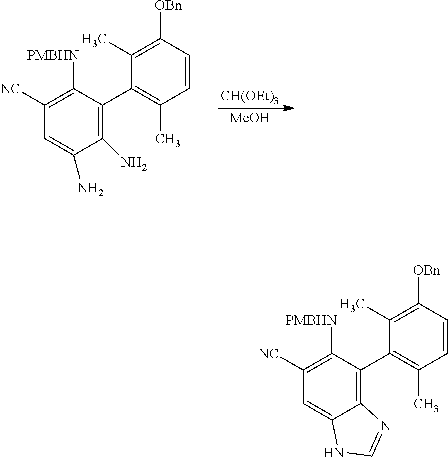

To a solution of 4,5-diamino-3-(3-benzyloxy-2,6-dimethyl-phenyl)-2-[(4-methoxyphenyl)methylamino]benzonitrile (10 g, 20.89 mmol, 1 eq) in MeOH (100 mL) was added triethyl orthoformate (4.64 g, 31.34 mmol, 5.21 mL, 1.5 eq). The mixture was stirred at 85° C. for 16 hours and then concentrated to remove methanol. The resulting residue was purified by column chromatography (SiO₂, 0-45% ethyl acetate in petroleum ether) to afford 7-(3-benzyloxy-2,6-dimethyl-phenyl)-6-[(4-methoxyphenyl)methylamino]-3H-benzimidazole-5-carbonitrile (7.44 g, 13.94 mmol, 67% yield, 91.55% purity). LCMS: m/z 479.3 [M+H]⁺.

### Step 2: 7-(3-Benzyloxy-2,6-dimethyl-phenyl)-3-(2-fluorophenyl)-6-[(4-methoxyphenyl)methylamino]benzimidazole-5-carbonitrile

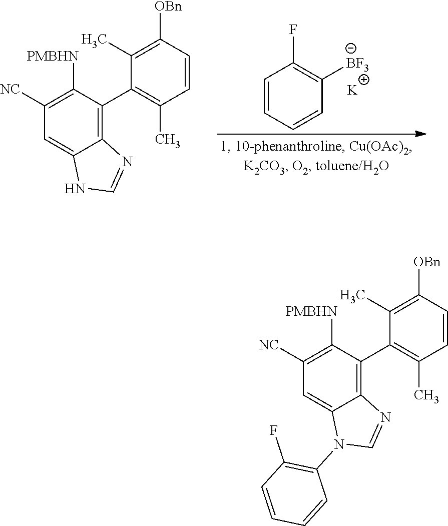

To a solution of 7-(3-benzyloxy-2,6-dimethyl-phenyl)-6-[(4-methoxyphenyl)-methylamino]-3H-benzimidazole-5-carbonitrile (1.7 g, 2.89 mmol, 1 eq, 3 batches) and potassium; trifluoro-(2-fluorophenyl)boranuide (2.92 g, 14.44 mmol, 5 eq) in toluene (20 mL) and H₂O (4 mL) were added Cu(OAc)₂ (157 mg, 0.87 mmol, 0.3 eq), 1,10-phenanthroline hydrate (86 mg, 0.43 mmol, 0.15 eq), and K₂CO₃ (798 mg, 5.78 mmol, 2 eq). The mixture was stirred at 80° C. for 16 hours under O₂ (15 Psi). The reaction mixture was diluted with water (50 mL) and extracted with ethyl acetate (50 mL×3). The combined organic layers were washed with aqueous saturated NaCl (30 mL×2), dried over anhydrous Na₂SO₄, filtered, and concentrated to give a residue. The residue was purified by column chromatography (SiO₂, 0-40% ethyl acetate in petroleum ether) to afford 7-(3-benzyloxy-2,6-dimethyl-phenyl)-3-(2-fluorophenyl)-6-[(4-methoxyphenyl)methylamino]-benzimidazole-5-carbonitrile (3.5 g, 5.34 mmol, 62% yield, 88.97% purity). LCMS: m/z 583.3 [M+H]⁺.

### Step 3: 6-Amino-7-(3-benzyloxy-2,6-dimethyl-phenyl)-3-(2-fluorophenyl)benz-imidazole-5-carbonitrile

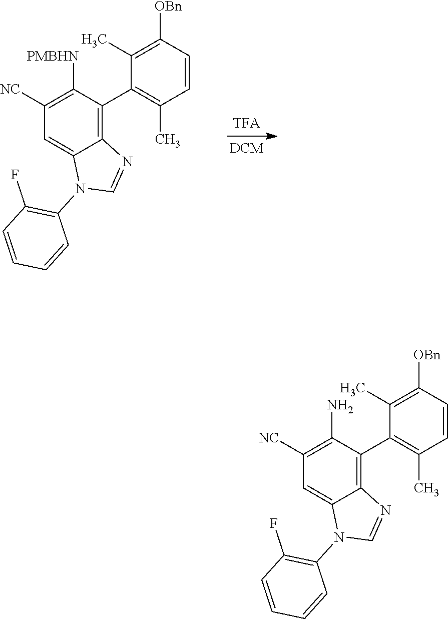

To a solution of 7-(3-benzyloxy-2,6-dimethyl-phenyl)-3-(2-fluorophenyl)-6-[(4-methoxyphenyl)methylamino]benzimidazole-5-carbonitrile (3.5 g, 6.01 mmol, 1 eq) in DCM (40 mL) was added TFA (20 mL) at 0° C. The mixture was stirred at 25° C. for 1 hour. The reaction was diluted with water (50 mL) and adjusted to pH=8 with aqueous saturated NaHCO₃. The residue was extracted with dichloromethane (50 mL×3). The combined organic layers were dried over anhydrous Na₂SO₄, filtered, and concentrated to give 6-amino-7-(3-benzyloxy-2,6-dimethyl-phenyl)-3-(2-fluorophenyl)benzimidazole-5-carbonitrile (2.8 g, 5.60 mmol, 93% yield, 92.50% purity), which was used in the next step without further purification. LCMS: m/z 463.2 [M+H]⁺.

### Step 4: 6-Amino-7-(3-benzyloxy-2,6-dimethyl-phenyl)-3-(2-fluorophenyl)benzimidazole-5-carboxamide

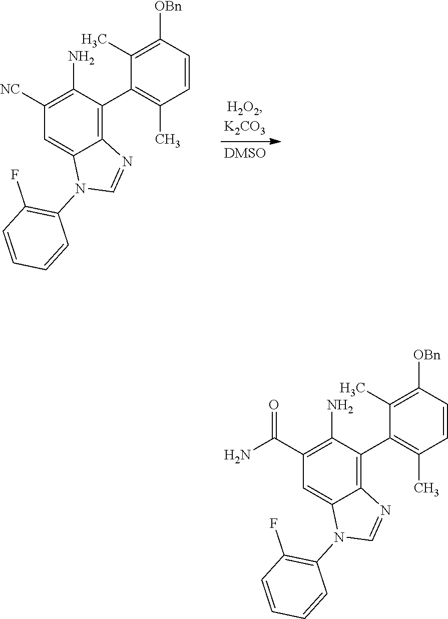 [SMILES: Cc1ccc(OCc2ccccc2)c(C)c1-c1c(N)c(C#N)cc2c1ncn2-c1ccccc1F ; Cc1ccc(OCc2ccccc2)c(C)c1-c1c(N)c(C(N)=O)cc2c1ncn2-c1ccccc1F]

To a solution of 6-amino-7-(3-benzyloxy-2,6-dimethyl-phenyl)-3-(2-fluorophenyl)benzimidazole-5-carbonitrile (3.1 g, 6.70 mmol, 1 eq) and K₂CO₃ (1.39 g, 10.05 mmol, 1.5 eq) in DMSO (40 mL) was added H₂O₂ (7.60 g, 67.02 mmol, 6.44 mL, 30% purity, 10 eq) at 0° C. The mixture was stirred at 20° C. for 16 hours. The reaction was adjusted to pH=7 with 1N HCl and quenched with aqueous saturated Na₂SO₃ (60 mL). The residue was extracted with ethyl acetate (50 mL×3). The combined organic layers were washed with aqueous saturated Na₂SO₃ (30 mL×2), dried over anhydrous Na₂SO₄, filtered, and concentrated to give a residue. The residue was purified by column chromatography (SiO₂, 0-50% ethyl acetate in petroleum ether) to give 6-amino-7-(3-benzyloxy-2,6-dimethyl-phenyl)-3-(2-fluorophenyl)benzimidazole-5-carboxamide (2.5 g, 5.19 mmol, 77% yield, 99.77% purity). LCMS: m/z 481.6 [M+H]⁺.

### Step 5: 6-Amino-3-(2-fluorophenyl)-7-(3-hydroxy-2,6-dimethyl-phenyl)benzimidazole-5-carboxamide

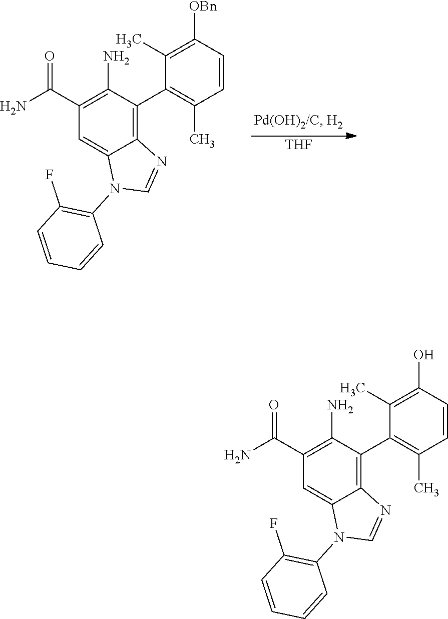 [SMILES: Cc1ccc(OCc2ccccc2)c(C)c1-c1c(N)c(C(N)=O)cc2c1ncn2-c1ccccc1F ; Cc1ccc(O)c(C)c1-c1c(N)c(C(N)=O)cc2c1ncn2-c1ccccc1F ; "THF"]

To a solution of 6-amino-7-(3-benzyloxy-2,6-dimethyl-phenyl)-3-(2-fluorophenyl)benzimidazole-5-carboxamide (4 g, 8.32 mmol, 1 eq) in THF (50 mL) was added Pd(OH)₂/C (4 g, 5.70 mmol, 20% purity, 0.68 eq). The mixture was stirred at 25° C. for 3 hours under H₂ (15 Psi). The reaction was filtered and washed with MeOH (50 mL). The collected material was triturated with MeOH (20 mL) at 25° C. for 30 min to give 6-amino-3-(2-fluorophenyl)-7-(3-hydroxy-2,6-dimethyl-phenyl)benzimidazole-5-carboxamide (2.8 g, 6.81 mmol, 82% yield, 95% purity). LCMS: m/z 391.1 [M+H]⁺.

### Step 6: Chiral Separation

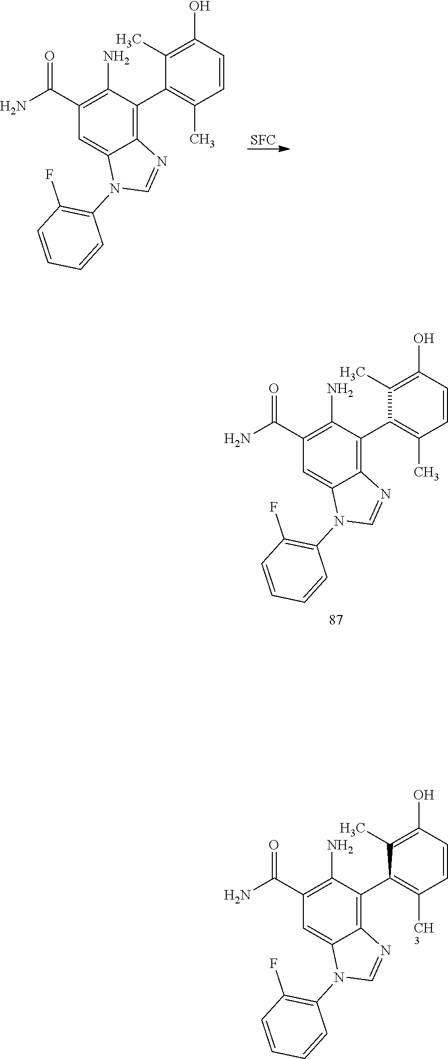

The racemate (2.8 g, 6.81 mmol) was purified by SFC (column: Daicel Chiralcel OX (250 mm×50 mm, 10 μm); mobile phase: [A: 60% CO₂, B: 40% EtOH (+0.1% NH₃H₂O)], isocratic elution mode) to afford (P)-6-amino-3-(2-fluorophenyl)-7-(3-hydroxy-2,6-dimethyl-phenyl)benzimidazole-5-carboxamide (87, 1.35 g, 3.45 mmol, 48% yield, 99.89% purity).

LCMS: m/z 391.2 [M+H]⁺. ¹H NMR: (400 MHz, DMSO-d₆) δ=9.10 (s, 1H), 8.28 (s, 1H), 7.94 (s, 1H), 7.82 (t, 1H), 7.66 (s, 1H), 7.61-7.53 (m, 2H), 7.50-7.40 (m, 1H), 7.22 (s, 1H), 6.98 (d, 1H), 6.79 (d, 1H), 5.36 (s, 2H), 1.81 (s, 3H), 1.74 (s, 3H). ¹⁹F NMR: (400 MHz, DMSO-d₆) δ=−122.84

### Example 76. (P)-3-cyano-4-(3-hydroxy-2,6-dimethylphenyl)-1-isopropyl-1H-pyrrolo[2,3-b]pyridine-6-carboxamide (88)

### Step 1: 4-Bromo-1-isopropyl-pyrrolo[2,3-b]pyridine-6-carbonitrile

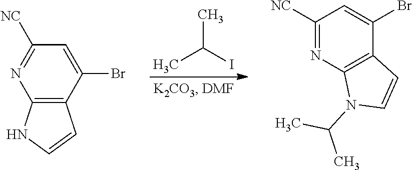

To a solution of 4-bromo-1H-pyrrolo[2,3-b]pyridine-6-carbonitrile (10 g, 45.04 mmol, 1 eq) and K₂CO₃ (18.67 g, 135.11 mmol, 3 eq) in DMF (80 mL) was added 2-iodopropane (11.48 g, 67.55 mmol, 6.74 mL, 1.5 eq). The mixture was stirred at 20° C. for 16 hours. The reaction mixture was quenched by addition H₂O (100 mL) at 20° C., and then diluted with ethyl acetate (300 mL) and extracted with ethyl acetate (300 mL×3). The combined organic layers were washed with aqueous saturated NaCl (1000 mL×2), dried over anhvdrous Na₂SO₄, filtered, and concentrated to give a residue, which was purified by column chromatography (SiO₂, 0-5% ethyl acetate in petroleum ether) to give 4-bromo-1-isopropyl-pyrrolo[2,3-b]pyridine-6-carbonitrile (8.5 g, 31.54 mmol, 70% yield, 98% purity). ¹H NMR: (400 MHz, DMSO-d₆)) δ=8.24 (d, 1H), 8.09 (s, 1H), 6.71 (d, 1H), 5.13 (m, 1H), 1.56 (s, 3H), 1.54 (s, 3H).

### Step 2: 4-(3-Benzyloxy-2,6-dimethyl-phenyl)-1-isopropyl-pyrrolo[2,3-b]pyridine-6-carbonitrile

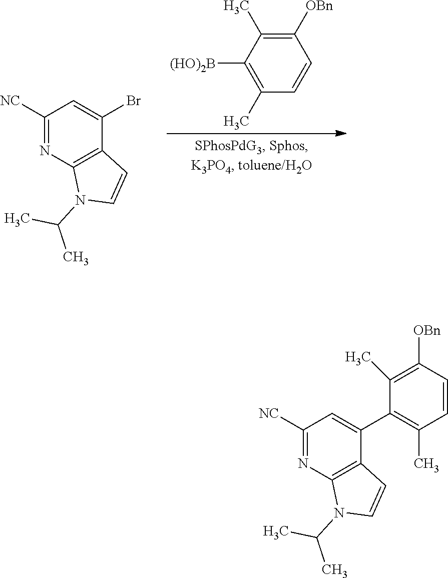 [SMILES: CC(C)n1ccc2c(Br)cc(C#N)nc21 ; Cc1ccc(OCc2ccccc2)c(C)c1B(O)O ; Cc1ccc(OCc2ccccc2)c(C)c1-c1cc(C#N)nc2c1ccn2C(C)C]

To a solution of 4-bromo-1-isopropyl-pyrrolo[2,3-b]pyridine-6-carbonitrile (8.5 g, 32.18 mmol, 1 eq) and (3-benzyloxy-2,6-dimethyl-phenyl) boronic acid (12.36 g, 48.27 mmol, 1.5 eq) in toluene (60 mL) and H₂O (12 mL) were added SPhos Pd G₃ (2.51 g, 3.22 mmol, 0.1 eq), SPhos (2.64 g, 6.44 mmol, 0.2 eq) and K₃PO₄ (20.49 g, 96.55 mmol, 3 eq). The mixture was stirred at 90° C. for 16 hours under nitrogen. The reaction mixture was quenched by addition H₂O (100 mL) at 20° C. and then extracted with ethyl acetate (300 mL×3). The combined organic layers were washed with aqueous saturated NaCl (500 mL×2), dried over anhydrous Na₂SO₄, filtered, and concentrated to afford a residue which was purified by column chromatography (SiO₂, 0-6% ethyl acetate in petroleum ether). The crude product was then triturated with petroleum ether:ethyl acetate (10:1, 20 mL) at 20° C. for 30 min to provide 4-(3-benzyloxy-2,6-dimethyl-phenyl)-1-isopropyl-pyrrolo[2,3-b]pyridine-6-carbonitrile (7 g, 17.70 mmol, 55% yield). ¹HNMR: (400 MHz, DMSO-d₆) δ=8.03 (d, 1H), 7.54-7.44 (m, 3H), 7.41 (m, 2H), 7.37-7.30 (m, 1H), 7.21-7.15 (m, 1H), 7.12-7.04 (m, 1H), 6.15 (d, 1H), 5.16 (m, 3H), 1.82 (d, 6H), 1.53 (d, 6H).

### Step 3: 4-(3-Benzyloxy-2,6-dimethyl-phenyl)-1-isopropyl-pyrrolo[2,3-b]pyridine-3,6-dicarbonitrile

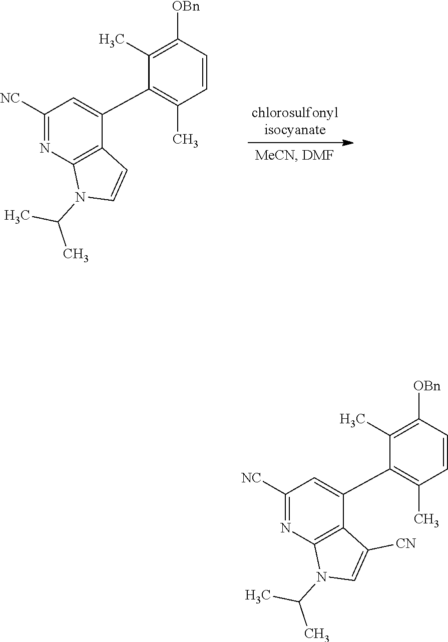 [SMILES: Cc1ccc(OCc2ccccc2)c(C)c1-c1cc(C#N)nc2c1ccn2C(C)C ; Cc1ccc(OCc2ccccc2)c(C)c1-c1cc(C#N)nc2c1c(C#N)cn2C(C)C]

To a solution of 4-(3-benzyloxy-2,6-dimethyl-phenyl)-1-isopropyl-pyrrolo[2,3-b]pyridine-6-carbonitrile (7 g, 17.70 mmol, 1 eq) in DMF (40 mL) was added a solution of N-(oxomethylene)sulfamoyl chloride (12.53 g, 88.50 mmol, 7.70 mL, 5 eq) in MeCN (120 mL) at −20° C. The mixture was stirred at 20° C. for 16 hours. The reaction mixture was quenched with H₂O (300 mL) at 20° C. and concentrated to remove MeCN. The residue was extracted with ethyl acetate (300 mL×3). The combined organic layers were washed with aqueous saturated NaCl (500 mL×2), dried over anhydrous Na₂SO₄, filtered, and concentrated to give a residue which was purified by column chromatography (SiO₂, 0-12% ethyl acetate in petroleum ether) to afford 4-(3-benzyloxy-2,6-dimethyl-phenyl)-1-isopropyl-pyrrolo[2,3-b]pyridine-3,6-dicarbonitrile (6.5 g, 15.46 mmol, 87% yield). LCMS: m/z 443.1 [M+H]⁺. ¹HNMR: (400 MHz, DMSO-d₆) δ=8.98 (s, 1H), 7.77 (s, 1H), 7.53-7.45 (m, 2H), 7.41 (m, 2H), 7.37-7.28 (m, 1H), 7.21-7.13 (m, 1H), 7.13-7.04 (m, 1H), 5.22-5.08 (m, 3H), 1.83 (d, 6H), 1.56 (d, 6H).

### Step 4: 4-(3-benzyloxy-2,6-dimethyl-phenyl)-3-cyano-1-isopropyl-pyrrolo[2,3-b]pyridine-6-carboxamide

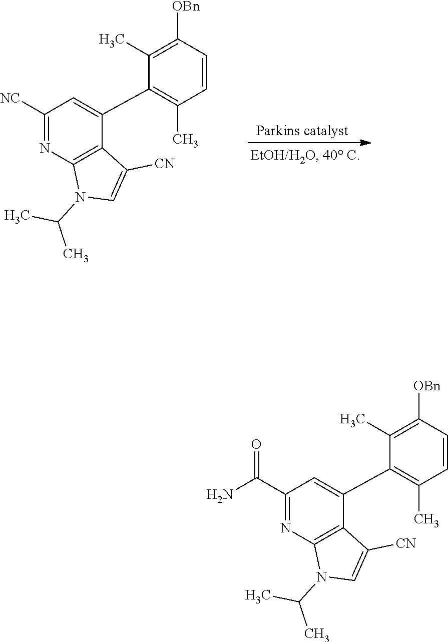 [SMILES: Cc1ccc(OCc2ccccc2)c(C)c1-c1cc(C#N)nc2c1c(C#N)cn2C(C)C ; Cc1ccc(OCc2ccccc2)c(C)c1-c1cc(C(N)=O)nc2c1c(C#N)cn2C(C)C]

To a solution of 4-(3-benzyloxy-2,6-dimethyl-phenyl)-1-isopropyl-pyrrolo[2,3-b]pyridine-3,6-dicarbonitrile (6.5 g, 15.46 mmol, 1 eq) in EtOH (40 mL) and H₂O (10 mL) was added Ghaffar-Parkins catalyst (1.32 g, 3.09 mmol, 0.2 eq). The mixture was stirred at 40° C. for 8 hours. The reaction mixture was quenched with H₂O (100 mL) at 20° C. and extracted with EtOAc (300 mL×3). The combined organic layers were washed with aqueous saturated NaCl (100 mL×2), dried over anhydrous Na₂SO₄, filtered, and concentrated to provide a residue which was purified by column chromatography (SiO₂, 0-40% ethyl acetate in petroleum ether) to afford 4-(3-benzyloxy-2,6-dimethyl-phenyl)-3-cyano-1-isopropyl-pyrrolo[2,3-b]pyridine-6-carboxamide (5.2 g, 11.50 mmol, 74.41% yield, 97% purity). LCMS: m/z 439.2 [M+H]⁻.

### Step 5: 3-Cyano-4-(3-hydroxy-2,6-dimethyl-phenyl)-1-isopropyl-pyrrolo[2,3-b]pyridine-6-carboxamide

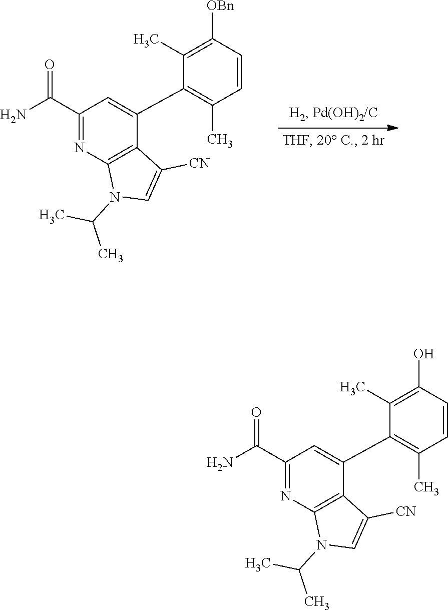 [SMILES: Cc1ccc(OCc2ccccc2)c(C)c1-c1cc(C(N)=O)nc2c1c(C#N)cn2C(C)C ; Cc1ccc(O)c(C)c1-c1cc(C(N)=O)nc2c1c(C#N)cn2C(C)C]

To a solution of 4-(3-benzyloxy-2,6-dimethyl-phenyl)-3-cyano-1-isopropyl-pyrrolo[2,3-b]pyridine-6-carboxamide (5 g, 11.40 mmol, 1 eq) in THF (100 mL) was added Pd(OH)₂/C (2 g, 2.85 mmol, 20% purity, 0.25 eq). The mixture was stirred at 20° C. for 2 hours under hydrogen. The reaction mixture was concentrated to remove solvent, and the resulting residue was purified by column chromatography (SiO₂, 0-60% ethyl acetate in petroleum ether). The crude product was triturated with MeOH (10 mL) at 20° C. for 30 min to afford 3-cyano-4-(3-hydroxy-2,6-dimethyl-phenyl)-1-isopropyl-pyrrolo[2,3-b]pyridine-6-carboxamide (3 g, 8.44 mmol, 74.01% yield, 98% purity). LCMS: m/z 349.1 [M+H]⁺.

### Step 6: Chiral Separation

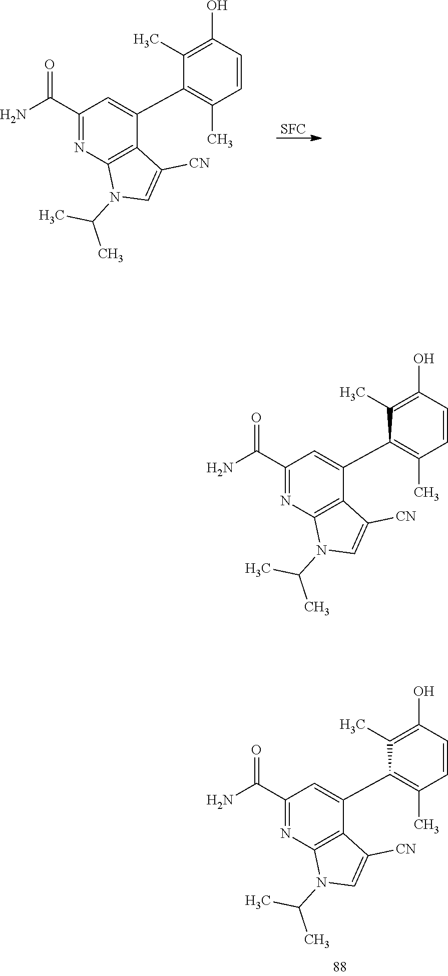

The residue (3.8 g) was purified by SFC (column: Daicel Chiralpak AD (250 mm×50 mm, 10 um); mobile phase[A 55% CO₂, B %: i-PrOH (0.1% NH—H₂O) 45%, isocratic elution mode) to afford (P)-3-cyano-4-(3-hydroxy-2,6-dimethyl-phenyl)-1-isopropyl-pyrrolo[2,3-b]pyridine-6-carboxamide (88, 1147.0 mg, 3.23 mmol, 74% yield, 99.57% purity.

LCMS: m/z 349.1 [M+H]⁺. ¹H NMR (400 MHz, DMSO-d₆) δ=9.27 (s, 1H), 8.81 (s, 1H), 8.36 (d, 1H), 7.83-7.47 (m, 2H), 6.97 (d, 1H), 6.81 (d, 1H), 5.54-5.31 (m, 1H), 1.84-1.69 (m, 6H), 1.55 (d, 6H).

### Example 77. (P)-3-cyano-1-ethyl-4-(3-hydroxy-2,6-dimethylphenyl-4-d)-1H-pyrrolo[2,3-b]pyridine-6-carboxamide (89)

### Step 1: 4-Bromo-1H-pyrrolo[2,3-b]pyridine-6-carboxylic acid

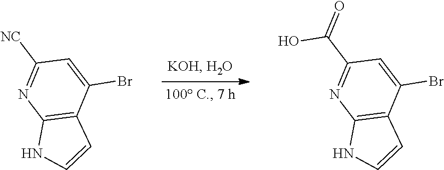 [SMILES: N#Cc1cc(Br)c2cc[nH]c2n1 ; O=C(O)c1cc(Br)c2cc[nH]c2n1]

To a solution of 4-bromo-1H-pyrrolo[2,3-b]pyridine-6-carbonitrile (2 g, 9.007 mmol, 1 equiv) in H₂O (20 mL) was added KOH (10.11 g, 180.140 mmol, 20 equiv). The mixture was stirred for 7 h at 100° C. The mixture was acidified to pH 6 with HCl (aqueous). The resulting mixture was extracted with EtOAc (3×100 mL). The combined organic layers were washed with water (3×10 mL) and dried over anhydrous Na₂SO₄. After filtration, the filtrate was concentrated under reduced pressure. The resulting residue was purified by silica gel column chromatography, eluting with PE/EA (1:1) to afford 4-bromo-1H-pyrrolo[2,3-b]pyridine-6-carboxylic acid (2 g, 92.12%).

### Step 2: Ethyl 4-bromo-1-ethylpyrrolo[2,3-b]pyridine-6-carboxylate

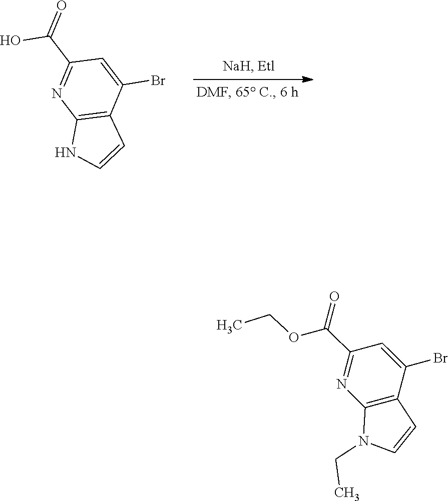 [SMILES: O=C(O)c1cc(Br)c2cc[nH]c2n1 ; CCOC(=O)c1cc(Br)c2ccn(CC)c2n1]

To a solution of 4-bromo-1H-pyrrolo[2,3-b]pyridine-6-carboxylic acid (2 g, 8.297 mmol, 1 equiv) in DMF (30 mL) was added NaH (0.60 g, 24.891 mmol, 3 equiv) at 0° C. The mixture was stirred for 30 min. Ethyl iodide (3.88 g, 24.891 mmol, 3 equiv) was added, and the mixture was allowed to warm to 65° C. and stirred for 6 h. The reaction mixture was quenched by water and extracted with EtOAc (3×25 mL). The crude product was purified by silica gel column chromatography, eluting with PE/EA (1:1) to afford ethyl 4-bromo-1-ethylpyrrolo[2,3-b]pyridine-6-carboxylate (2.1 g, 85.17%).

### Step 3: Ethyl 4-bromo-3-cyano-1-ethylpyrrolo[2,3-b]pyridine-6-carboxylate

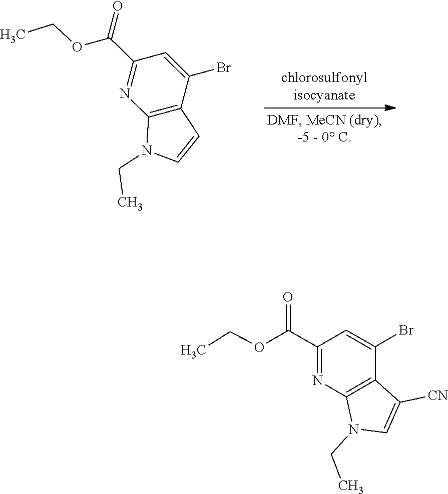 [SMILES: CCOC(=O)c1cc(Br)c2ccn(CC)c2n1 ; CCOC(=O)c1cc(Br)c2c(C#N)cn(CC)c2n1]

To a solution of ethyl 4-bromo-1-ethylpyrrolo[2,3-b]pyridine-6-carboxylate (2.1 g, 7.067 mmol, 1 equiv) in MeCN (20 mL) was added chlorosulfonyl isocyanate (5.00 g, 35.335 mmol, 5 equiv) in MeCN (20 mL) at −5° C. The mixture was stirred for 10 min. DMF (20 mL) was added and the mixture was allowed to warm to 0° C. and stirred for 30 min. The resulting mixture was extracted with EtOAc (3×100 mL). The combined organic layers were washed with water (3×30 mL) and dried over anhydrous Na₂SO₄. After filtration, the filtrate was concentrated under reduced pressure. This resulted in ethyl 4-bromo-3-cyano-1-ethylpyrrolo[2,3-b]pyridine-6-carboxylate (550 mg, 24.16%).

### Step 4: 2-Bromo-5-iodo-4-methoxy-1,3-dimethylbenzene

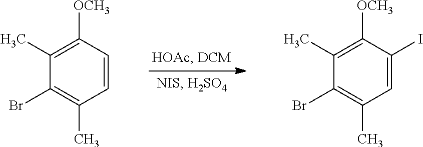

Into a 500 mL round-bottom flask were added 2-bromo-4-methoxy-1,3-dimethylbenzene (10 g, 46.492 mmol, 1 equiv), HOAc (60 mL), DCM (60 mL), H₂SO₄ (45.59 g, 464.920 mmol, 10 equiv), and N-iodosuccinimide (10.46 g, 46.492 mmol, 1 equiv). The resulting mixture was stirred for 12 h at room temperature under nitrogen. The mixture was basified to pH 8 with saturated NaHCO (aqueous). The aqueous layer was extracted with CH₂Cl₂ (50 mL×2). The residue was purified by silica gel column chromatography, eluting with PE (100%) to afford 2-bromo-5-iodo-4-methoxy-1,3-dimethylbenzene (14 g, 88.31%).

### Step 5: 2-Bromo-4-methoxy-1,3-dimethyl(5-2H) benzene

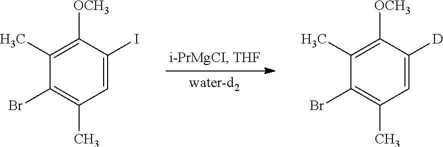

To a stirred solution of 2-bromo-5-iodo-4-methoxy-1,3-dimethylbenzene (3 g, 8.798 mmol, 1 equiv) in THF (25 mL) was added i-PrMgCl (8.80 mL, 17.596 mmol, 2 equiv) dropwise at −40° C. under nitrogen. The resulting mixture was stirred for 15 min at −40° C. under nitrogen. To the above mixture was added D₂O (352.40 mg, 17.596 mmol, 2 equiv) dropwise at 0° C. The resulting mixture was stirred for an additional 12 h at room temperature. The reaction was quenched by the addition of saturated NH₄Cl (aqueous) (2 mL) at room temperature. The aqueous layer was extracted with EtOAc (50 mL×2). The residue was purified by silica gel column chromatography, eluting with PE/EA (99:1) to afford 2-bromo-4-methoxy-1,3-dimethyl(5-2H) benzene (1.35 g, 71.01%).

### Step 6: 3-Methoxy-2,6-dimethyl(4-²H) phenylboronic acid

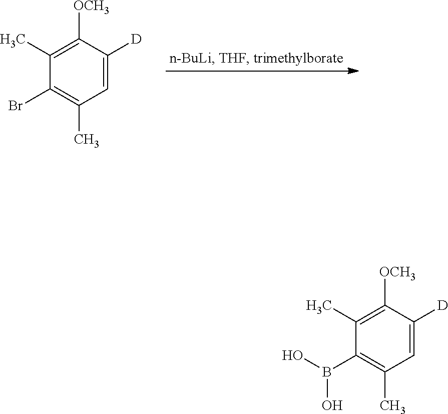

To a stirred solution of 2-bromo-4-methoxy-1,3-dimethyl(5-²H) benzene (1.5 g, 6.941 mmol, 1 equiv) in THF (20 mL) was added n-BuLi (3.61 mL, 9.025 mmol, 1.30 equiv) dropwise at −78° C. under nitrogen. The resulting mixture was stirred for 1 h at −78° C. under nitrogen. To the above mixture was added trimethyl borate (1.01 g, 9.717 mmol, 1.4 equiv) dropwise at −78° C. The resulting mixture was stirred for an additional 12 h at room temperature. The reaction was quenched by the addition of saturated NH₄Cl (aqueous) (20 ML) at room temperature. The aqueous layer was extracted with EtOAc (50 mL×3). The residue was purified by reversed-phase flash chromatography with the following conditions: column, C18 silica gel; mobile phase, MeOH in Water, 30% to 35% gradient in 40 min; detector, UV 220 nm. The procedure resulted in 3-methoxy-2,6-dimethyl(4-²H) phenylboronic acid (800 mg, 63.67%). ¹H NMR (400 MHz, DMSO-d6) δ 8.11 (s, 2H), 6.89 (s, 1H), 3.71 (s, 3H), 2.19 (s, 3H), 2.09 (s, 3H).

### Step 7: Ethyl 3-cyano-1-ethyl-4-[3-methoxy-2,6-dimethyl(4-²H)phenyl]pyrrolo[2,3-b]pyridine-6-carboxylate

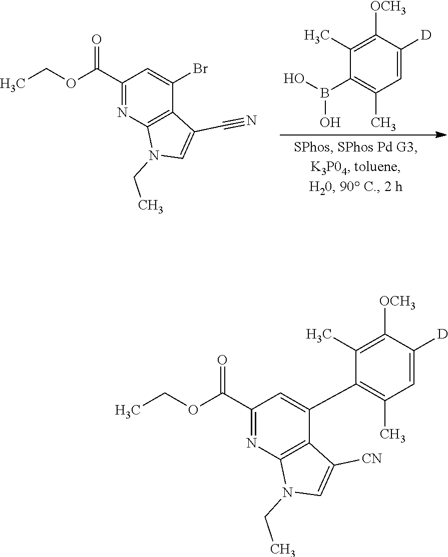 [SMILES: CCOC(=O)c1cc(Br)c2c(C#N)cn(CC)c2n1 ; [2H]c1cc(C)c(B(O)O)c(C)c1OC ; [2H]c1cc(C)c(-c2cc(C(=O)OCC)nc3c2c(C#N)cn3CC)c(C)c1OC]

To a solution of ethyl 4-bromo-3-cyano-1-ethylpyrrolo[2,3-b]pyridine-6-carboxylate (545 mg, 1.692 mmol, 1 equiv) and 3-methoxy-2,6-dimethyl(4-²H) phenylboronic acid (367.48 mg, 2.030 mmol, 1.2 equiv) in toluene (10 mL) and H₂O (2 mL) were added K₃PO₄ (718.18 mg, 3.384 mmol, 2 equiv), SPhos (138.90 mg, 0.338 mmol, 0.2 equiv) and SPhos Pd Gen.3 (132.00 mg, 0.169 mmol, 0.1 equiv). After stirring for 2 h at 90° C. under nitrogen, the mixture was extracted with EtOAc (3×30 mL). The combined organic layers were washed with water (3×10 mL) and dried over anhydrous Na₂SO₄. After filtration, the filtrate was concentrated under reduced pressure. The residue was purified by silica gel column chromatography, eluted with PE/EA (1:1) to afford ethyl 3-cyano-1-ethyl-4-[3-methoxy-2,6-dimethyl(4-²H)phenyl]pyrrolo[2,3-b]pyridine-6-carboxylate (500 mg, 78.10%).

### Step 8: 3-Cyano-1-ethyl-4-[3-methoxy-2,6-dimethyl(4-2H) phenyl]pyrrolo[2,3-b]pyridine-6-carboxamide

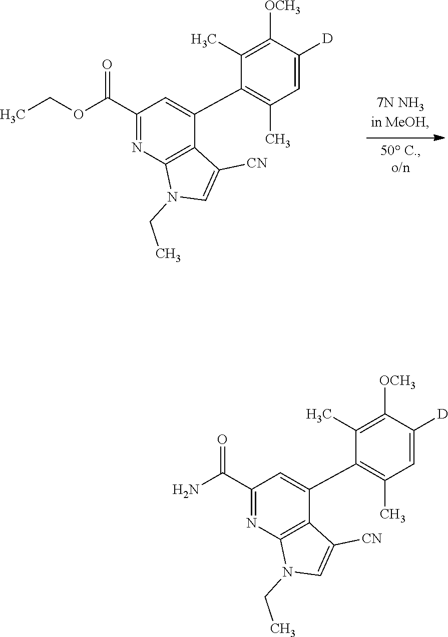 [SMILES: [2H]c1cc(C)c(-c2cc(C(=O)OCC)nc3c2c(C#N)cn3CC)c(C)c1OC ; [2H]c1cc(C)c(-c2cc(C(N)=O)nc3c2c(C#N)cn3CC)c(C)c1OC]

To a solution of ethyl 3-cyano-1-ethyl-4-[3-methoxy-2,6-dimethyl(4-²H) phenyl]pyrrolo[2,3-b]pyridine-6-carboxylate (490 mg, 1.295 mmol, 1 equiv) was added NH₃(g) in MeOH (12 mL). The mixture was stirred overnight at 50° C. and then concentrated under reduced pressure. This resulted in 3-cyano-1-ethyl-4-[3-methoxy-2,6-dimethyl(4-2H) phenyl]pyrrolo[2,3-b]pyridine-6-carboxamide (460 mg, crude).

### Step 9: 3-Cyano-1-ethyl-4-(3-hydroxy-2,6-dimethylphenyl-4-²H)-1H-pyrrolo[2,3-b]pyridine-6-carboxamide

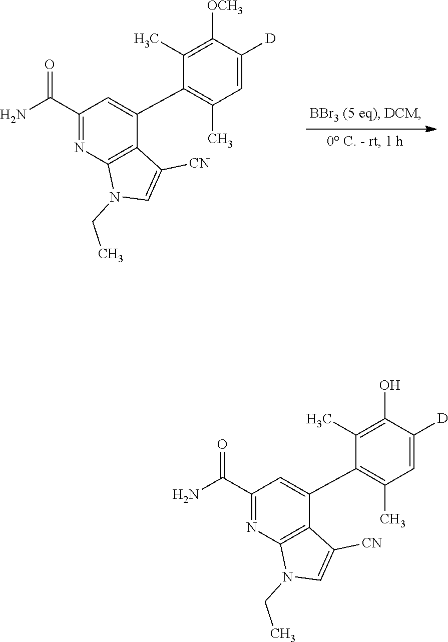 [SMILES: [2H]c1cc(C)c(-c2cc(C(N)=O)nc3c2c(C#N)cn3CC)c(C)c1OC ; [2H]c1cc(C)c(-c2cc(C(N)=O)nc3c2c(C#N)cn3CC)c(C)c1O]

To a solution of 3-cyano-1-ethyl-4-[3-methoxy-2,6-dimethyl(4-²H) phenyl]pyrrolo[2,3-b]pyridine-6-carboxamide (100 mg, 0.286 mmol, 1 equiv) in DCM (2 mL) was added BBr₃ (358.49 mg, 1.430 mmol, 5 equiv) at 0° C. The resulting mixture was diluted with MeOH (30 mL) at 0° C. The mixture was basified to pH 8 with saturated NaHCO₃(aqueous). The resulting mixture was extracted with EtOAc (3×20 mL). The combined organic layers were washed with brine (2×20 mL) and dried over anhydrous Na₂SO₄. After filtration, the filtrate was concentrated under reduced pressure. The mixture was stirred for 1 h at rt. The crude product was purified by preparative scale HPLC (Mobile Phase A: Water (10 mmol/L NH₄HCO₃+0.05% NH₃·H₂O), Mobile Phase B: MeCN; Gradient: 24% B to 35% B in 11 min) to afford 3-cyano-1-ethyl-4-(3-hydroxy-2,6-dimethylphenyl-4-²H)-1H-pyrrolo[2,3-b]pyridine-6-carboxamide (42.9 mg, 44.7%). LCMS (m/z): [M+1]=336.

¹H NMR (300 MHz, DMSO-d6) δ 9.24 (s, 1H), 8.69 (s, 1H), 8.36-8.30 (m, 1H), 7.71 (s, 1H), 7.64 (s, 1H), 6.96 (s, 1H), 4.51 (q, J=7.2 Hz, 2H), 1.80-1.74 (m, 3H), 1.70 (s, 3H), 1.50 (t, J=7.2 Hz, 3H).

### Step 10: Chiral Separation

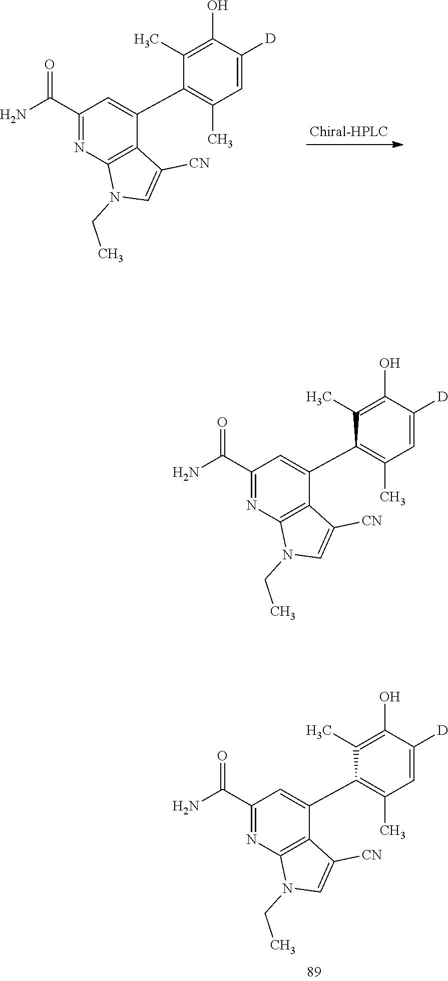

The crude product (170 mg) was purified by Chiral-Prep-HPLC to afford (P)-3-cyano-1-ethyl-4-(3-hydroxy-2,6-dimethylphenyl-4-d)-1H-pyrrolo[2,3-b]pyridine-6-carboxamide (89, 39 mg, rear peak). Conditions: Column: CHIRALPAK ID, 2×25 cm, 5 μm; Mobile Phase A: Hex(0.5% 2M NH₃-MeOH)-HPLC, Mobile Phase B: EtOH-HPLC; Flow rate: 20 mL/min; Gradient: isocratic 20; wavelength: 220/254 nm; RT1(min): 11.343; RT2(min): 14.903: Sample Solvent: EtOH-HPLC; Injection Volume: 0.4 mL; Number of Runs: 25.

LCMS: (ES, m/z): [M+1]=336.15. ¹H NMR (300 MHz, DMSO-d6) δ 9.24 (s, 1H), 8.69 (s, 1H), 8.33 (s, 1H), 7.71 (s, 1H), 7.64 (s, 1H), 6.96 (s, 1H), 4.51 (q, J=7.2 Hz, 2H), 1.77 (s, 3H), 1.70 (s, 3H), 1.50 (t, J=7.2 Hz, 3H).

### Example 78. (M)-5-amino-4-(3-hydroxy-2-methylphenyl)-1-methyl-3-(1H-1,2,3-triazol-4-yl)-1H-pyrrolo[2,3-b]pyridine-6-carboxamide (90)

### Step 1: 5-(Benzhydrylideneamino)-4-(3-benzyloxy-2-methyl-phenyl)-1-methyl-3-(1-tetrahydropyran-2-yltriazol-4-yl)pyrrolo[2,3-b]pyridine-6-carbonitrile

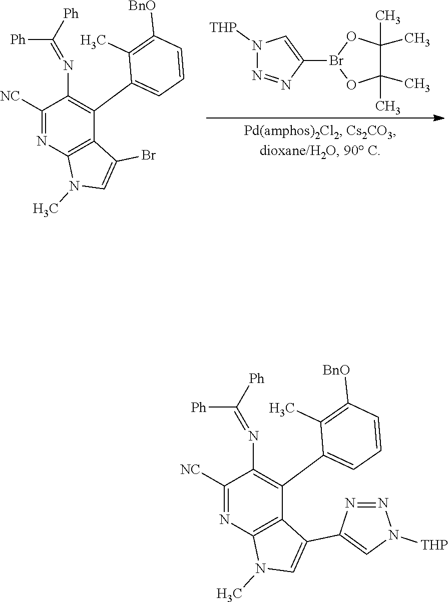

To a solution of 5-(benzhydrylideneamino)-4-(3-benzyloxy-2-methyl-phenyl)-3-bromo-1-methyl-pyrrolo[2,3-b]pyridine-6-carbonitrile (500 mg, 0.87 mmol, 1 eq) and 1-tetrahydropyran-2-yl-4-(4,4,5,5-tetramethyl-1,3,2-dioxaborolan-2-yl)triazole (456 mg, 1.64 mmol, 2 eq) in dioxane (6 mL) and H₂O (1.5 mL) was added 4-ditert-butylphosphanyl-N,N-dimethyl-aniline; dichloropalladium (57 mg, 0.08 mmol, 0.1 eq) and Cs₂CO₃ (799 mg, 2.45 mmol, 3 eq). The mixture was stirred at 90° C. for 4 hour. The reaction mixture was diluted with water (100 mL) and extracted with ethyl acetate (80 mL×3). The combined organic layers were dried over anhydrous Na₂SO₄, filtered and concentrated to give a residue. The residue was purified by column chromatography (SiO₂, 0-30% ethyl acetate in petroleum ether) to give 5-(benzhydrylideneamino)-4-(3-benzyloxy-2-methyl-phenyl)-1-methyl-3-(1-tetrahydropyran-2-yltriazol-4-yl)pyrrolo[2,3-b]pyridine-6-carbonitrile (522 mg, 0.68 mmol, 84.03% yield, 90% purity). LCMS: m/z 684.3 [M+H]⁺

### Step 2: 5-Amino-4-(3-benzyloxy-2-methyl-phenyl)-1-methyl-3-(1H-triazol-4-yl)pyrrolo[2,3-b]pyridine-6-carbonitrile

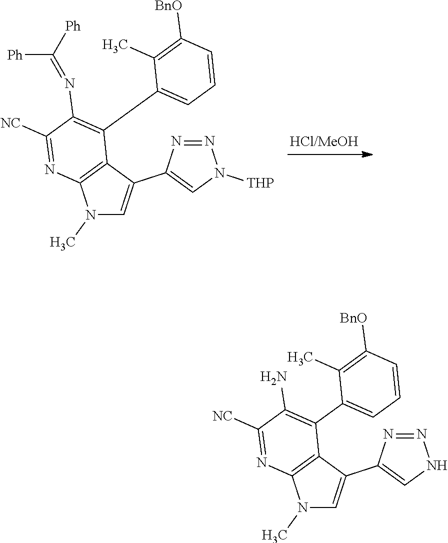

A solution of 5-(benzhydrylideneamino)-4-(3-benzyloxy-2-methyl-phenyl)-1-methyl-3-(1-tetrahydropyran-2-yltriazol-4-yl)pyrrolo[2,3-b]pyridine-6-carbonitrile (820 mg, 1.20 mmol, 1 eq) in HCl/MeOH (8 mL) was stirred at 15° C. for 16 hours. The mixture was concentrated to remove solvent. The residue was purified by column chromatography (SiO₂, 0-80% ethyl acetate in petroleum ether) to give 5-amino-4-(3-benzyloxy-2-methyl-phenyl)-1-methyl-3-(1H-triazol-4-yl)pyrrolo[2,3-b]pyridine-6-carbonitrile (488 mg, 1.08 mmol, 89.71% yield, 96% purity). LCMS: m/z 436.0 [M+3]⁺

### Step 3: 5-Amino-4-(3-benzyloxy-2-methyl-phenyl)-1-methyl-3-(1H-triazol-4-yl)pyrrolo[2,3-b]pyridine-6-carboxamide

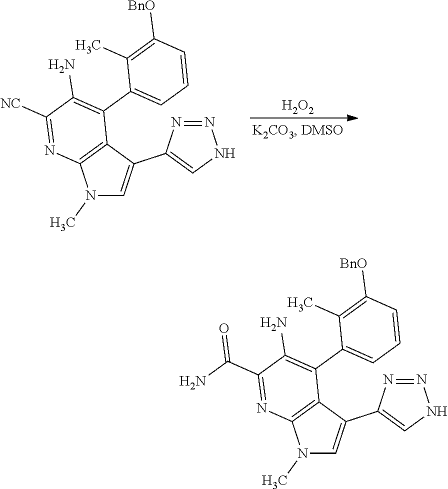 [SMILES: Cc1c(OCc2ccccc2)cccc1-c1c(N)c(C#N)nc2c1c(-c1c[nH]nn1)cn2C ; "H2O2" ; Cc1c(OCc2ccccc2)cccc1-c1c(N)c(C(N)=O)nc2c1c(-c1c[nH]nn1)cn2C]

To a solution of 5-amino-4-(3-benzyloxy-2-methyl-phenyl)-1-methyl-3-(1H-triazol-4-yl)pyrrolo[2,3-b]pyridine-6-carbonitrile (488 mg, 1.12 mmol, 1 eq) in DMSO (5 mL) were added H₂O₂ (1.46 g, 12.88 mmol, 1.24 mL, 30% purity, 11.49 eq) and K₂CO₃ (309 mg, 2.24 mmol, 2 eq) at 0° C. The mixture was stirred at 25° C. for 2 hours. The reaction was diluted with water (50 mL) and quenched with aqueous saturated Na₂SO₃ (50 mL). The residue was filtered to give 5-amino-4-(3-benzyloxy-2-methyl-phenyl)-1-methyl-3-(1H-triazol-4-yl)pyrrolo[2,3-b]pyridine-6-carboxamide (450 mg, 0.97 mmol, 86.22% yield, 97.37% purity). LCMS: m/z 454.2 [M+H]⁺

### Step 4: 5-Amino-4-(3-hydroxy-2-methyl-phenyl)-1-methyl-3-(1H-triazol-4-yl)pyrrolo[2,3-b]pyridine-6-carboxamide

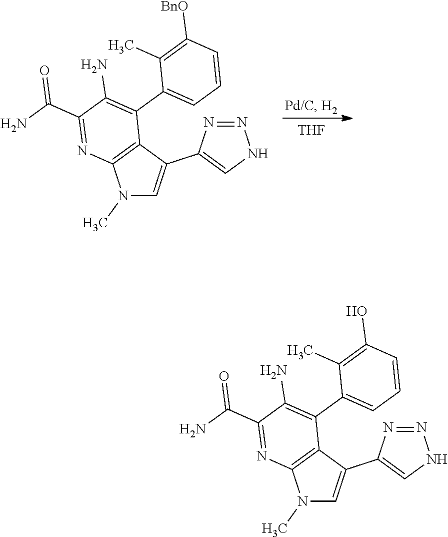 [SMILES: Cc1c(OCc2ccccc2)cccc1-c1c(N)c(C(N)=O)nc2c1c(-c1c[nH]nn1)cn2C ; "Pd/C, H2" ; Cc1c(O)cccc1-c1c(N)c(C(N)=O)nc2c1c(-c1c[nH]nn1)cn2C ; "THF"]

To a solution of 5-amino-4-(3-benzyloxy-2-methyl-phenyl)-1-methyl-3-(1H-triazol-4-yl)pyrrolo[2,3-b]pyridine-6-carboxamide (400 mg, 0.88 mmol, 1 eq) in THF (6 mL) was added Pd/C (500 mg, 10% purity). The mixture was stirred at 25° C. for 2 hours under H₂ (15 psi). The reaction was filtered and concentrated to give the crude product. The crude product was triturated with MeOH (20 mL×2) at 25 for ° C. 30 min to give 5-amino-4-(3-hydroxy-2-methyl-phenyl)-1-methyl-3-(1H-triazol-4-yl)pyrrolo[2,3-b]pyridine-6-carboxamide (229.9 mg, 0.61 mmol, 69.49% yield, 96.87% purity). LCMS: m/z 364.1 [M+H]. ¹H NMR: (400 MHz, DMSO-d₆) δ=14.51 (s, 1H), 9.47 (s, 1H), 8.25 (d, 1H), 7.91 (s, 1H), 7.44 (d, 1H), 7.07 (t, 1H), 6.89 (d, 1H), 6.55 (d, 1H), 5.79 (s, 1H), 5.70 (s, 2H), 3.88 (s, 3H), 1.65 (s, 3H).

### Step 5: Chiral Separation

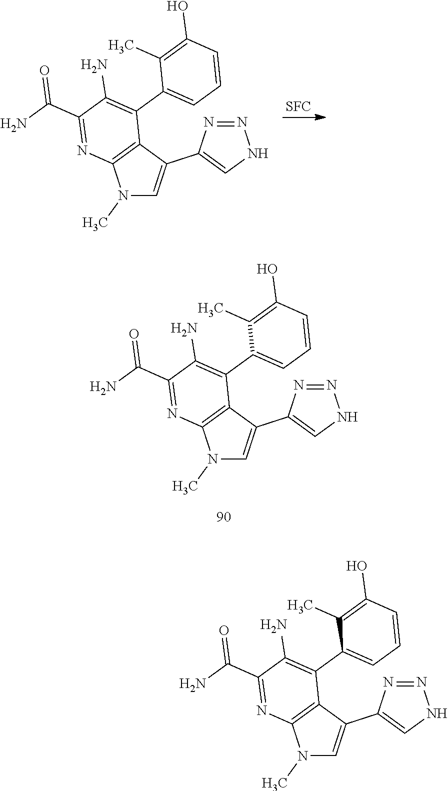

The racemate (200 mg) was purified by SFC to give (M)-5-amino-4-(3-hydroxy-2-methyl-phenyl)-1-methyl-3-(1H-triazol-4-yl)pyrrolo[2,3-b]pyridine-6-carboxamide (90, 43.6 mg, 0.12 mmol, 22.15% yield, 96.54% purity). Conditions: DAICEL CHIRALPAK AS (250 mm×30 mm, 10 μm); mobile phase: [CO₂-MeOH (0.1% NH₃H₂O)]; B %:45%, isocratic elution mode). LCMS: m/z 364.1 [M+H]⁺. ¹H NMR: (400 MHz, DMSO-d6) δ=8.25 (d, 1H), 7.91 (s, 1H), 7.49-7.39 (m, 1H), 7.07 (t, 1H), 6.89 (d, 1H), 6.55 (d, 1H), 5.77 (s, 1H), 5.69 (s, 2H), 3.87 (s, 3H), 1.65 (s, 3H).

### Example 79. (M)-5-amino-1-ethyl-4-(3-hydroxy-2-methylphenyl)-3-(1H-pyrazol-4-yl)-1H-pyrrolo[2,3-b]pyridine-6-carboxamide (91)

### Step 1: 5-Bromo-4-chloro-1-ethyl-pyrrolo[2,3-b]pyridine-6-carbonitrile

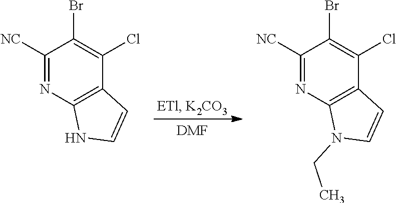

To a solution of 5-bromo-4-chloro-1H-pyrrolo[2,3-b]pyridine-6-carbonitrile (5.5 g, 21.44 mmol, 1 eq) in DMF (50 mL) was added CH₃CH₂I (4.01 g, 25.74 mmol, 2.06 mL, 1.2 eq) and K₂CO₃ (8.89 g, 64.33 mmol, 3 eq). The mixture was stirred at 25° C. for 2 hours. The reaction mixture was diluted with water (100 mL) and filtered to give 5-bromo-4-chloro-1-ethyl-pyrrolo[2,3-b]pyridine-6-carbonitrile (4.19 g, 11.49 mmol, 53% yield, 78% purity). LCMS: m/z 284.1 [M+H]⁺.

### Step 2: 5-(Benzhydrylideneamino)-4-chloro-1-ethyl-pyrrolo[2,3-b]pyridine-6-carbonitrile

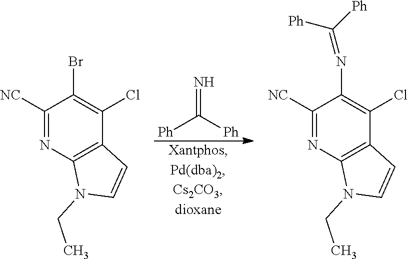 [SMILES: CCn1ccc2c(Cl)c(Br)c(C#N)nc21 ; N=C(c1ccccc1)c1ccccc1 ; "Xantphos,Pd(dba)2,Cs2CO3,dioxane" ; CCn1ccc2c(Cl)c(N=C(c3ccccc3)c3ccccc3)c(C#N)nc21]

To a solution of 5-bromo-4-chloro-1-ethyl-pyrrolo[2,3-b]pyridine-6-carbonitrile (4.1 g, 14.41 mmol, 1 eq) and diphenylmethanimine (2.74 g, 15.13 mmol, 2.54 mL, 1.05 eq) in dioxane (50 mL) were added Xantphos (833 mg, 1.44 mmol, 0.1 eq), Pd(dba)₂ (830 mg, 1.44 mmol, 0.1 eq) and Cs₂CO₃ (14.08 g, 43.23 mmol, 3 eq). The mixture was stirred at 100° C. for 16 hours under nitrogen. The reaction mixture was filtered and concentrated to remove solvent. The residue was purified by column chromatography (SiO₂. 0-10% ethyl acetate in petroleum ether) to give 5-(benzhydrylideneamino)-4-chloro-1-ethyl-pyrrolo[2,3-b]pyridine-6-carbonitrile (1.6 g, 3.78 mmol, 26% yield, 91% purity). LCMS: m/z 384.9 [M+H]⁺.

### Step 3: 5-(benzhydrylideneamino)-4-(3-benzyloxy-2-methyl-phenyl)-1-ethyl-pyrrolo[2,3-b]pyridine-6-carbonitrile

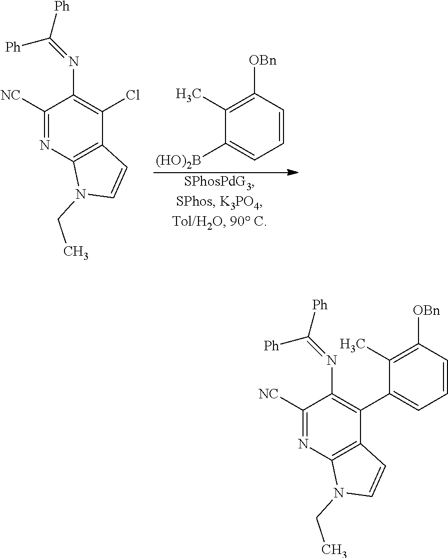 [SMILES: CCn1ccc2c(Cl)c(N=C(c3ccccc3)c3ccccc3)c(C#N)nc21 ; Cc1c(OCc2ccccc2)cccc1B(O)O ; "SPhosPdG3,SPhos, K3PO4,Tol/H2O, 90° C." ; CCn1ccc2c(-c3cccc(OCc4ccccc4)c3C)c(N=C(c3ccccc3)c3ccccc3)c(C#N)nc21]

To a solution of 5-(benzhydrylideneamino)-4-chloro-1-ethyl-pyrrolo[2,3-b]pyridine-6-carbonitrile (1.6 g, 4.16 mmol, 1 eq) and (3-benzyloxy-2-methyl-phenyl)boronic acid (1.51 g, 6.23 mmol, 1.5 eq) in toluene (40 mL) and H₂O (8 mL) were added SPhos Pd G₃ (325 mg, 0.42 mmol, 0.1 eq), SPhos (341 mg, 0.83 mmol, 0.2 eq) and K₃PO₄ (2.65 g, 12.47 mmol, 3 eq). The mixture was stirred at 90° C. for 16 hours under nitrogen. The reaction mixture was concentrated to remove solvent. The residue was diluted with water (50 mL) and extracted with ethyl acetate (50 mL×3). The combined organic layers were washed with aqueous saturated NaCl (40 mL×2), dried over anhydrous Na₂SO₄, filtered and concentrated to give a residue. The residue was purified by column chromatography (SiO₂, 0-60% ethyl acetate in petroleum ether) to give 5-(benzhydrylideneamino)-4-(3-benzyloxy-2-methyl-phenyl)-1-ethyl-pyrrolo[2,3-b]pyridine-6-carbonitrile (2.09 g, 3.75 mmol, 90% yield, 98% purity). LCMS: m/z 547.4 [M+H]⁺.

### Step 4: 5-(Benzhydrylideneamino)-4-(3-benzyloxy-2-methyl-phenyl)-3-bromo-1-ethyl-pyrrolo[2,3-b]pyridine-6-carbonitrile

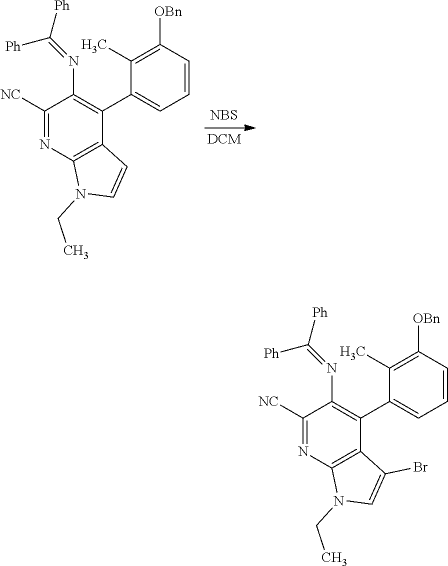

To a solution of 5-(benzhvdrylideneamino)-4-(3-benzyloxy-2-methyl-phenyl)-1-ethyl-pyrrolo[2,3-b]pyridine-6-carbonitrile (1.99 g, 3.64 mmol, 1 eq) in DCM (20 mL) was added NBS (647 mg, 3.64 mmol, 1 eq). The mixture was stirred at 25° C. for 12 hours. The reaction mixture was concentrated to remove solvent. The residue was diluted with water (40 mL) and extracted with ethyl acetate (40 mL×3). The combined organic layers were washed with aqueous saturated NaCl (40 mL×2), dried over anhydrous Na₂SO₄, filtered, and concentrated to give 5-(benzhydrylideneamino)-4-(3-benzyloxy-2-methyl-phenyl)-3-bromo-1-ethyl-pyrrolo[2,3-b]pyridine-6-carbonitrile (2.2 g, crude). LCMS: m/z 627.3 [M+H]⁺.

### Step 5: 5-(Benzhydrylideneamino)-4-(3-benzyloxy-2-methyl-phenyl)-1-ethyl-3-(1-tetrahydropyran-2-ylpyrazol-4-yl)pyrrolo[2,3-b]pyridine-6-carbonitrile

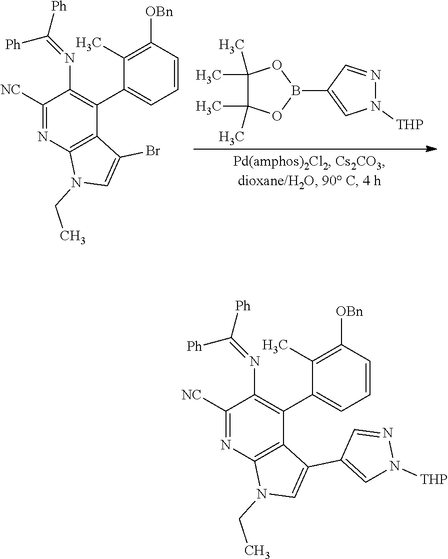

To a solution of 5-(benzhydrylideneamino)-4-(3-benzyloxy-2-methyl-phenyl)-3-bromo-1-ethyl-pyrrolo[2,3-b]pyridine-6-carbonitrile (2 g, 3.20 mmol, 1 eq) and 1-tetrahydropyran-2-yl-4-(4,4,5,5-tetramethyl-1,3,2-dioxaborolan-2-yl)pyrazole (1.07 g, 3.84 mmol, 1.2 eq) in dioxane (36 mL) and H₂O (9 mL) were added 4-ditert-butylphosphanyl-N,N-dimethyl-aniline:dichloropalladium (227 mg, 0.320 mmol, 0.25 mL, 0.1 eq) and Cs₂CO₃ (3.12 g, 9.59 mmol, 3 eq). The mixture was stirred at 90° C. for 4 hours under nitrogen. The reaction mixture was concentrated to remove solvent, and the resulting residue was diluted with water (50 mL) and extracted with ethyl acetate (50 mL×3). The combined organic layers were washed with aqueous saturated NaCl (60 mL×2), dried over anhydrous Na₂SO₄, filtered, and concentrated. The crude material was purified by column chromatography (SiO₂, 0-35% ethyl acetate in petroleum ether) to give 5-(benzhydrylideneamino)-4-(3-benzyloxy-2-methyl-phenyl)-1-ethyl-3-(1-tetrahydropyran-2-ylpyrazol-4-yl)pyrrolo[2,3-b]pyridine-6-carbonitrile (1.6 g, 2.04 mmol, 63.92% yield, 89% purity). LCMS: m/z 697.4 [M+H]⁺.

### Step 6: 5-Amino-4-(3-benzyloxy-2-methyl-phenyl)-1-ethyl-3-(1H-pyrazol-4-yl)pyrrolo[2,3-b]pyridine-6-carbonitrile

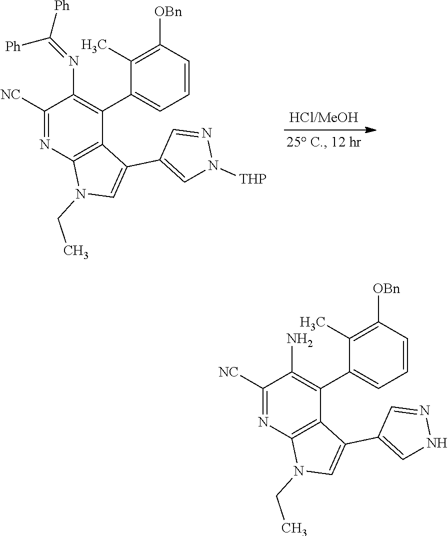

A solution of 5-(benzhydrylideneamino)-4-(3-benzyloxy-2-methyl-phenyl)-1-ethyl-3-(1-tetrahydropyran-2-ylpyrazol-4-yl)pyrrolo[2,3-b]pyridine-6-carbonitrile (1.5 g, 2.15 mmol, 1 eq) in HCl/MeOH (20 mL) was stirred at 25° C. for 12 hours. The reaction mixture was diluted with water (40 mL) and adjusted to pH=7-8 with aqueous NaHCO₃. The mixture was extracted with ethyl acetate (50 mL×3). The combined organic layers were washed with aqueous saturated NaCl (50 mL×2), dried over anhydrous Na₂SO₄, filtered, and concentrated to give a residue. The residue was purified by column chromatography (SiO₂, 0-60% ethyl acetate in petroleum ether) to give 5-amino-4-(3-benzyloxy-2-methyl-phenyl)-1-ethyl-3-(1H-pyrazol-4-yl)pyrrolo[2,3-b]pyridine-6-carbonitrile (700 mg, 1.47 mmol, 68% yield, 94% purity). LCMS: m/z 449.1 [M+H]⁺.

### Step 7: 5-Amino-4-(3-benzyloxy-2-methyl-phenyl)-1-ethyl-3-(1H-pyrazol-4-yl)pyrrolo[2,3-b]pyridine-6-carboxamide

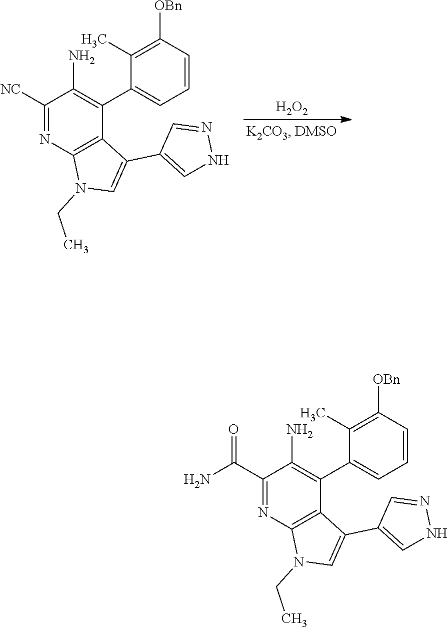 [SMILES: CCn1cc(-c2cn[nH]c2)c2c(-c3cccc(OCc4ccccc4)c3C)c(N)c(C#N)nc21 ; "H2O2" ; CCn1cc(-c2cn[nH]c2)c2c(-c3cccc(OCc4ccccc4)c3C)c(N)c(C(N)=O)nc21]

To a solution of 5-amino-4-(3-benzyloxy-2-methyl-phenyl)-1-ethyl-3-(1H-pyrazol-4-yl)pyrrolo[2,3-b]pyridine-6-carbonitrile (500 mg, 1.11 mmol, 1 eq) and K₂CO₃ (462 mg, 3.34 mmol, 3 eq) in DMSO (8 mL) was added H₂O₂ (3.5 g, 30.87 mmol, 2.97 mL, 30% purity, 27.69 eq) at 0° C. The mixture was stirred at 25° C. for 2 hours. The reaction mixture was dissolved in water (40 mL), then was adjusted to pH=7-8 with HCl (2M). The mixture was extracted with ethyl acetate (50 mL×3). The combined organic layers were washed with aqueous saturated NaCl (60 mL×3) and Na₂SO₃ (60 mL×3), dried over anhydrous Na₂SO₄, filtered, and concentrated to give 5-amino-4-(3-benzyloxy-2-methyl-phenyl)-1-ethyl-3-(1H-pyrazol-4-yl)pyrrolo[2,3-b]pyridine-6-carboxamide (530 mg, 0.75 mmol, 67% yield, 66% purity). LCMS: m/z 467.1[M+H]⁺.

### Step 8: 5-Amino-1-ethyl-4-(3-hydroxy-2-methyl-phenyl)-3-(1H-pyrazol-4-yl)pyrrolo[2,3-b]pyridine-6-carboxamide

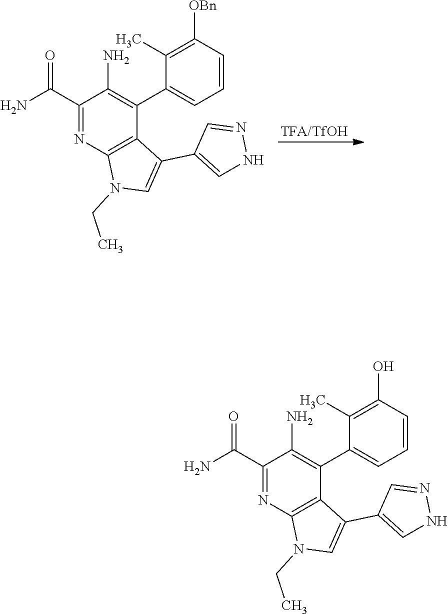 [SMILES: CCn1cc(-c2cn[nH]c2)c2c(-c3cccc(OCc4ccccc4)c3C)c(N)c(C(N)=O)nc21 ; "TFA/TfOH" ; CCn1cc(-c2cn[nH]c2)c2c(-c3cccc(O)c3C)c(N)c(C(N)=O)nc21]

To a solution of 5-amino-4-(3-benzyloxy-2-methyl-phenyl)-1-ethyl-3-(1H-pyrazol-4-yl)pyrrolo[2,3-b]pyridine-6-carboxamide (130 mg, 0.28 mmol, 1 eq) in TFA (1 mL) was added TfOH (0.3 mL) at 0° C. The mixture was stirred at 25° C. for 2 hours. The reaction mixture was diluted with water (40 mL) and adjusted to pH=7-8 with aqueous NaHCO₃. The mixture was extracted with ethyl acetate (40 mL×2). The combined organic layers were washed with aqueous saturated NaCl (100 mL×2), dried over anhydrous Na₂SO₄, filtered, and concentrated to give a residue. The residue was purified by prep-HPLC (column: XTIMATE C18 150×40 mm×10 μm; mobile phase: [water(FA)-ACN]:gradient:2%-42% B over 25 min) to give 5-amino-1-ethyl-4-(3-hydroxy-2-methyl-phenyl)-3-(1H-pyrazol-4-yl)pyrrolo[2,3-b]pyridine-6-carboxamide (101.3 mg, 0.26 mmol, 94% yield, 98% purity). LCMS: m/z 377.2 [M+H]⁺. ¹HNMR: (400 MHz, DMSO-d₆) δ=12.36-12.19 (m, 1H), 9.41-9.36 (m, 1H), 8.23-8.17 (m, 1H), 7.73-7.69 (m, 1H), 7.37 (d, 1H), 7.08-7.03 (m, 1H), 6.85 (d, 1H), 6.55 (d, 1H), 5.59 (s, 2H), 4.31 (q, 2H), 1.64 (s, 3H), 1.42 (t, 3H)

### Step 9: Chiral Separation

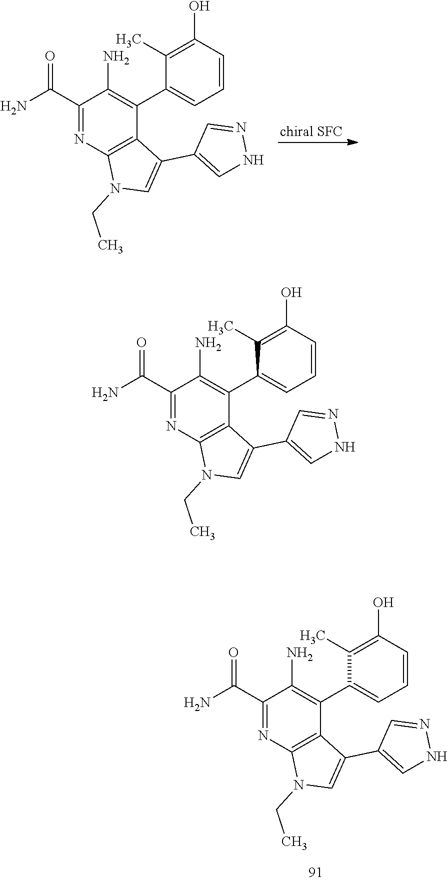

The racemate (380 mg) was purified by chiral SFC to give (M)-5-amino-1-ethyl-4-(3-hydroxy-2-methyl-phenyl)-3-(1H-pyrazol-4-yl)pyrrolo[2,3-b]pyridine-6-carboxamide (91, 180.4 mg, 0.475 mmol, 47.13% yield, 99.28% purity). Conditions: column: DAICEL CHIRALPAK IC (250 mm×30 mm, 10 μm):mobile phase: [CO₂-EtOH (0.1% NH₃H₂O)]; B %:35%, isocratic elution mode. LCMS: m/z 377.1 [M+H]⁺. ¹HNMR: (400 MHz, DMSO-d₆) δ=12.27 (s, 1H), 9.37 (s, 1H), 8.19 (d, 1H), 7.71 (s, 1H), 7.37 (d, 1H), 7.06 (t, 1H), 6.85 (d, 2H), 6.55 (d, 2H), 5.59 (s, 2H), 4.37-4.23 (m, 2H), 1.64 (s, 3H), 1.42 (t, 3H).

### Example 80. (M)-5-amino-3-cyano-1-ethyl-4-(3-hydroxy-2-methylphenyl)-1H-pyrrolo[2,3-b]pyridine-6-carboxamide (92)

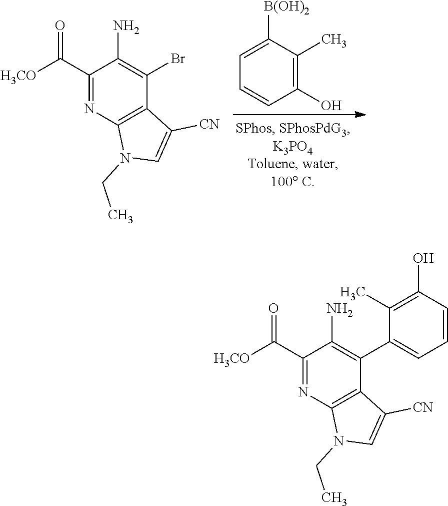 [SMILES: CCn1cc(C#N)c2c(Br)c(N)c(C(=O)OC)nc21 ; Cc1c(O)cccc1B(O)O ; CCn1cc(C#N)c2c(-c3cccc(O)c3C)c(N)c(C(=O)OC)nc21]

### Step 1: Methyl 5-amino-3-cyano-1-ethyl-4-(3-hydroxy-2-methylphenyl)-1H-pyrrolo[2,3-b]pyridine-6-carboxylate

To a mixture of methyl 5-amino-4-bromo-3-cyano-1-ethyl-pyrrolo[2,3-b]pyridine-6-carboxylate (150 mg, 0.46 mmol), (3-hydroxy-2-methyl-phenyl)boronic acid (130 mg, 0.85 mmol), dicyclohexyl-[2-(2,6-dimethoxyphenyl)phenyl]phosphane (40 mg, 0.097 mmol), [2-(2-aminophenyl)phenyl]-methylsulfonyloxy-palladium; dicyclohexyl-[2-(2,6-dimethoxyphenyl)phenyl]phosphane (74 mg, 0.095 mmol) and potassium phosphate (200 mg, 0.94 mmol) under inert atmosphere in a 2.5 mL microwave vial were added degassed toluene (3 mL) and water (0.3 mL). The vial was sealed and heated to 100° C. for 1.5 h. The mixture was cooled and diluted with ethyl acetate (10 mL) and water (3 mL). The organic layer was separated, and the aqueous layer was further extracted with ethyl acetate (3×10 mL). The combined organic layers were adsorbed onto Celite® for silica column chromatography using ethyl acetate/heptane as eluent. The product methyl 5-amino-3-cyano-1-ethyl-4-(3-hydroxy-2-methyl-phenyl)pyrrolo[2,3-b]pyridine-6-carboxylate eluted at 50% ethyl acetate/heptane (105 mg, 65% Yield). LC-MS m/z 351.10 (M+H⁺). ¹H NMR (400 MHz, DMSO) δ 9.54 (s, 1H), 8.55 (s, 1H), 7.14 (t, J=7.8 Hz, 1H), 6.92 (d, J=8.1 Hz, 1H), 6.61 (d, J=7.5 Hz, 1H), 5.68 (s, 2H), 4.26 (qd, J=6.9, 3.0 Hz, 2H), 3.89 (s, 3H), 1.80 (s, 3H), 1.44 (t, J=7.2 Hz, 3H).

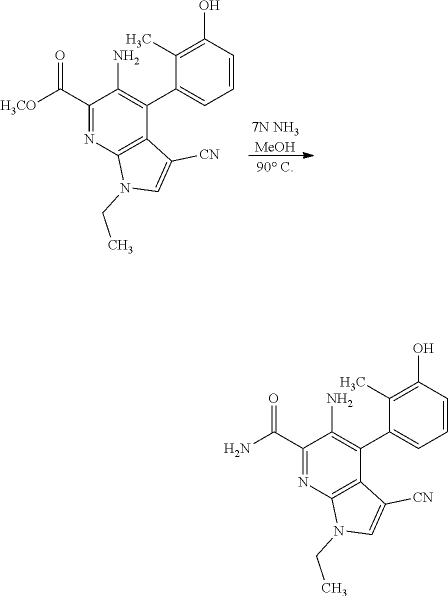

### Step 2: 5-amino-3-cyano-1-ethyl-4-(3-hydroxy-2-methylphenyl)-1H-pyrrolo[2,3-b]pyridine-6-carboxamide

A suspension of methyl 5-amino-3-cyano-1-ethyl-4-(3-hydroxy-2-methyl-phenyl)pyrrolo[2,3-b]pyridine-6-carboxylate (100 mg, 0.28 mmol) in 7N ammonia/methanol (2.5 mL, 18 mmol, 7 mol/L) was heated to 90° C. in a microwave for 12 h. The mixture was cooled and adsorbed onto Celite® and purified by silica column chromatography using MeOH and DCM as eluent. The product 5-amino-3-cyano-1-ethyl-4-(3-hydroxy-2-methylphenyl)-1H-pyrrolo[2,3-b]pyridine-6-carboxamide (65 mg, 68% yield) eluted with 5% MeOH in DCM. LCMS: m/z 336.10 (M+H⁺). ¹H NMR (400 MHz, DMSO) δ 9.50 (s, 1H), 8.47 (s, 1H), 8.31 (d, J=2.9 Hz, 1H), 7.55 (d, J=2.8 Hz, 1H), 7.13 (t, J=7.8 Hz, 1H), 6.90 (d, J=8.1 Hz, 1H), 6.60 (d, J=7.5 Hz, 1H), 5.95 (s, 2H), 4.44-4.28 (m, J=7.2 Hz, 2H), 1.81 (s, 3H), 1.42 (t, J=7.2 Hz, 3H).

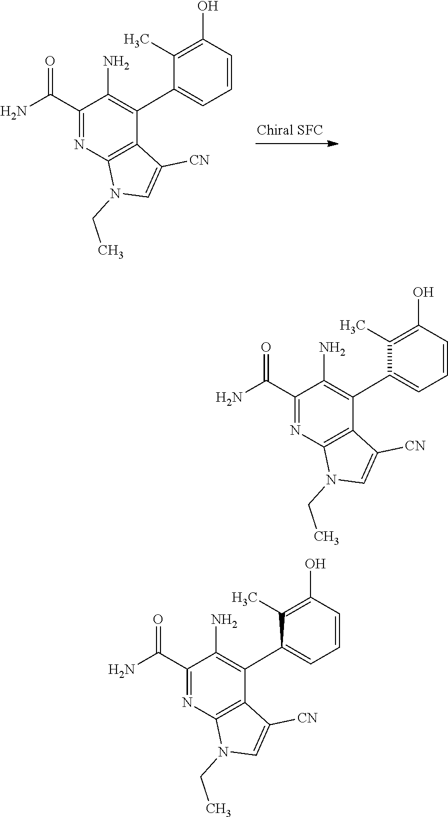

### Step 3: Chiral Separation

Separation of the racemate was conducted using chiral SFC. Column: DAICEL CHIRALPAK OJ, 30×150 mm, 5 μm; Conditions: Isocratic at 25% methanol with 75% CO2; Flow Rate: 100 mL/min) provided (M)-5-amino-3-cyano-1-ethyl-4-(3-hydroxy-2-methylphenyl)-1H-pyrrolo[2,3-b]pyridine-6-carboxamide (92). Retention time (iso25-OJ) 2.85 min. ¹H NMR (400 MHz, DMSO) δ 9.43 (s, 1H), 8.40 (s, 1H), 8.24 (d, J=2.9 Hz, 1H), 7.48 (d, J=2.9 Hz, 1H), 7.06 (t, J=7.8 Hz, 1H), 6.83 (d, J=8.1 Hz, 1H), 6.53 (d, J=7.6 Hz, 1H), 5.88 (s, 2H), 4.37-4.21 (m, J=7.0 Hz, 2H), 1.74 (s, 3H), 1.35 (t, J=7.2 Hz, 3H). LC-MS m/z 336.15 (M+H⁺).

### Example 81. (P)-3-cyano-1-(2,2-difluoroethyl)-4-(3-hydroxy-2,6-dimethylphenyl)-1H-pyrrolo[2,3-b]pyridine-6-carboxamide (93)

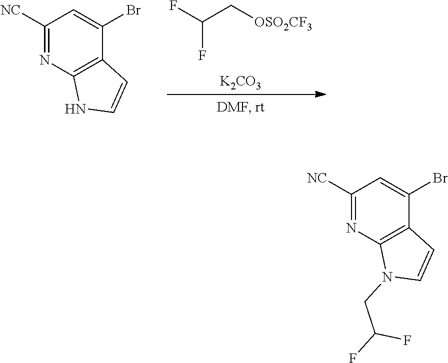

### Step 1: 4-Bromo-1-(2,2-difluoroethyl)-1H-pyrrolo[2,3-b]pyridine-6-carbonitrile

To a solution of 4-bromo-1H-pyrrolo[2,3-b]pyridine-6-carbonitrile (4.0 g, 18.01 mmol) and 2,2-difluoroethyl trifluoromethanesulfonate (5.1 g, 24 mmol) in N,N-dimethylformamide (70 mL) was added potassium carbonate (7.5 g, 54 mmol), and the mixture was stirred at room temperature overnight. The reaction was then quenched with ice cold water (200 mL) which caused formation of a solid precipitate. The precipitate was filtered and dried to the obtain desired product 4-bromo-1-(2,2-difluoroethyl)pyrrolo[2,3-b]pyridine-6-carbonitrile (4.9 g, 95% Yield) LC-MS m/z 286.05, 288.15 (M+H).

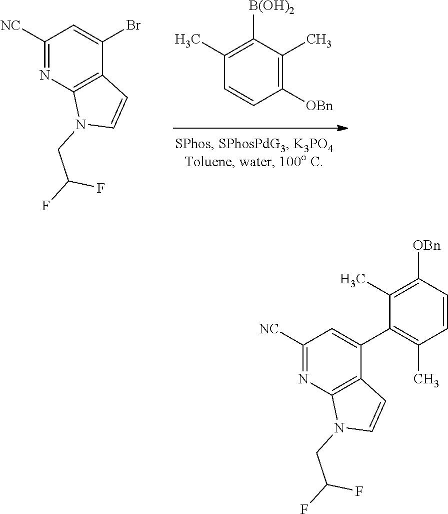 [SMILES: N#Cc1cc(Br)c2ccn(CC(F)F)c2n1 ; Cc1ccc(OCc2ccccc2)c(C)c1B(O)O ; Cc1ccc(OCc2ccccc2)c(C)c1-c1cc(C#N)nc2c1ccn2CC(F)F]

### Step 2: 4-(3-(benzyloxy)-2,6-dimethylphenyl)-1-(2,2-difluoroethyl)-1H-pyrrolo[2,3-b]pyridine-6-carbonitrile

To a 250-mL single neck round bottom flask under nitrogen was added 4-bromo-1-(2,2-difluoroethyl)pyrrolo[2,3-b]pyridine-6-carbonitrile (4.8 g, 17 mmol) dicyclohexyl-[2-(2,6-dimethoxyphenyl)phenyl]phosphane (1.4 g, 3.4 mmol), (3-benzyloxy-2,6-dimethyl-phenyl)boronic acid (6.4 g, 25 mmol), toluene (155 mL), and water (17 mL). The mixture was degassed with nitrogen for 5 mins. and then added [2-(2-aminophenyl) phenyl]-methylsulfonyloxy-palladium:dicyclohexyl-[2-(2,6-dimethoxyphenyl)phenyl]-phosphane (1.3 g, 1.7 mmol) and potassium phosphate (11.1 g, 52.3 mmol) were added. The flask was fitted with a condenser and stirred at 100° C. for 5 hr. The mixture was cooled and diluted with water (25 mL). The organic layer was separated and extracted with ethyl acetate (3×50 mL). The combined organic layer was washed with water and brine, dried over sodium sulphate, filtered through Celite®, and evaporated to dryness. The resulting crude material was dried overnight to obtain 4-(3-benzyloxy-2,6-dimethyl-phenyl)-1-(2,2-difluoroethyl)pyrrolo[2,3-b]pyridine-6-carbonitrile (11.3 g). LCMS: m/z 418.2 (M+H⁺).

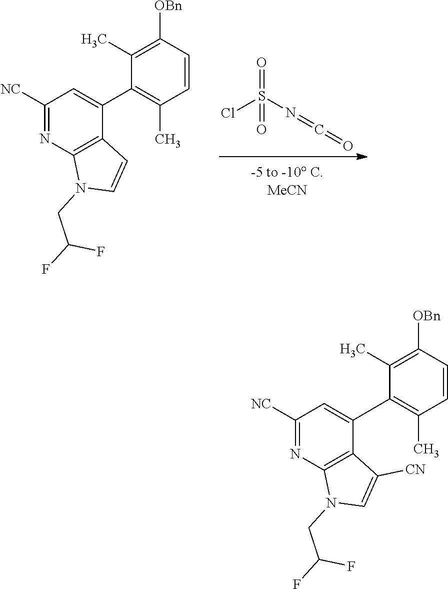 [SMILES: Cc1ccc(OCc2ccccc2)c(C)c1-c1cc(C#N)nc2c1ccn2CC(F)F ; O=C=NS(=O)(=O)Cl ; Cc1ccc(OCc2ccccc2)c(C)c1-c1cc(C#N)nc2c1c(C#N)cn2CC(F)F]

### Step 3: 4-(3-(benzyloxy)-2,6-dimethylphenyl)-1-(2,2-difluoroethyl)-1H-pyrrolo[2,3-b]pyridine-3,6-dicarbonitrile (Intermediate A3)

In a 500-mL single neck round bottomed flask, a solution of 4-(3-benzyloxy-2,6-dimethyl-phenyl)-1-(2,2-difluoroethyl)pyrrolo[2,3-b]pyridine-6-carbonitrile (7 g, 14.2 mmol, 85 mass %) in acetonitrile (180 mL) under nitrogen was cooled to −10° C. Then a solution of N-(oxomethylene)sulfamoyl chloride (6 mL, 69.1 mmol) in 5 mL of acetonitrile was added in two portions over 15 min. The resulting solution was stirred for 1.5 h, maintaining the temperature below −5° C. Then N,N-dimethylformamide (15 mL) was added over 30 sec and the mixture was stirred for 1 h. Workup was then conducted at 0° C. The mixture was diluted with 120 mL ethyl acetate in the open flask, and 8 mL of ammonium hydroxide solution was added dropwise. When the pH became basic, a yellow solid precipitated. Then water (50 mL) was added to dissolve the solids, and the organic layer was separated. The aqueous layer was evaporated and extracted with ethyl acetate and combined with the organic layer. The combined organic layer was washed with water and brine, dried over sodium sulphate, and evaporated to dryness. The crude material was dissolved in ethyl acetate and adsorbed onto silica for purification via silica column chromatography using a 330-g ISCO column, 100% hexane to ramping up to 30% ethyl acetate over 25 min. The desired product eluted around 25% ethyl acetate, and the fractions were dried to obtain 4-(3-benzyloxy-2,6-dimethyl-phenyl)-1-(2,2-difluoroethyl)pyrrolo[2,3-b]pyridine-3,6-dicarbonitrile (5.5 g, 87% yield). LCMS: m/z 465.1 (M+Na). ¹H NMR: (400 MHz, DMSO) δ 8.83 (s, 1H), 7.86 (s, 1H), 7.48 (d, J=7.1 Hz, 2H), 7.41 (t, J=7.7 Hz, 2H), 7.33 (t, J=7.3 Hz, 1H), 7.17 (d, J=8.6 Hz, 1H), 7.11 (d, J=8.5 Hz, 1H), 6.56 (tt, J=54.5, 3.3 Hz, 1H), 5.23-5.10 (m, 2H), 4.92 (td, J=15.8, 3.3 Hz, 2H), 1.85 (s, 3H), 1.82 (s, 3H).

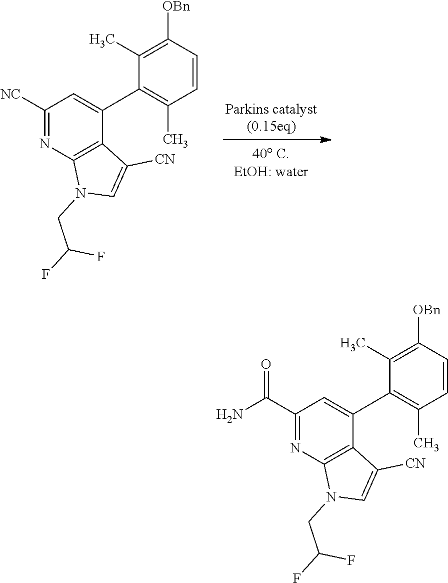 [SMILES: Cc1ccc(OCc2ccccc2)c(C)c1-c1cc(C#N)nc2c1c(C#N)cn2CC(F)F ; Cc1ccc(OCc2ccccc2)c(C)c1-c1cc(C(N)=O)nc2c1c(C#N)cn2CC(F)F]

### Step 4: 4-(3-(benzyloxy)-2,6-dimethylphenyl)-3-cyano-1-(2,2-difluoroethyl)-1H-pyrrolo[2,3-b]pyridine-6-carboxamide (Intermediate A4)

To a solution of 4-(3-benzyloxy-2,6-dimethyl-phenyl)-1-(2,2-difluoroethyl)pyrrolo[2,3-b]pyridine-3,6-dicarbonitrile (5.3 g, 12 mmol) in ethanol (170 mL) and water (15 mL) was added Ghaffar-Parkins catalyst (dimethylphosphinite; dimethylphosphinous acid; platinum(1+) monohydride 720 mg, 1.67 mmol) and stirred at 40° C. for 3 h. The mixture was cooled and solid particles containing mostly catalyst were removed via filtration. The filtrate was evaporated, and the crude material was dissolved in ethyl acetate (300 mL), washed with water and brine, and adsorbed onto silica for column chromatography using DCM/MeOH. The product eluted with 5% MeOH in DCM to obtain 4-(3-benzyloxy-2,6-dimethyl-phenyl)-3-cyano-1-(2,2-difluoroethyl)pyrrolo[2,3-b]pyridine-6-carboxamide (5.3 g 91% yield). LCMS m/z 461.1 (M+H⁺).

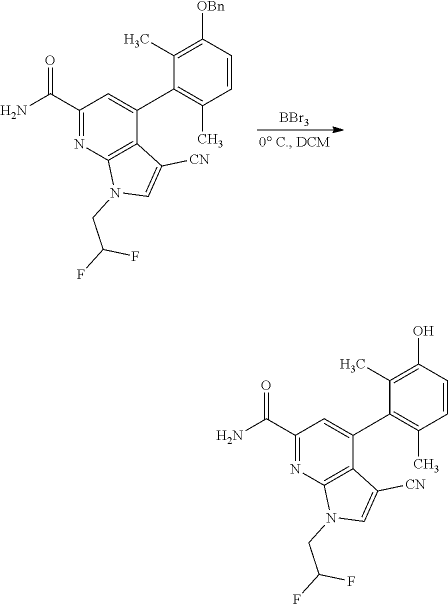

### Step 5: 3-cyano-1-(2,2-difluoroethyl)-4-(3-hydroxy-2,6-dimethylphenyl)-1H-pyrrolo[2,3-b]pyridine-6-carboxamide

Boron tribromide (0.6 mL, 0.6 mmol, 1M) was added dropwise to a solution of 4-(3-benzyloxy-2,6-dimethyl-phenyl)-3-cyano-1-(2,2-difluoroethyl)pyrrolo[2,3-b]pyridine-6-carboxamide (75 mg, 0.16 mmol) in dichloromethane (1 mL) in an ice bath. Immediately, orange solid began to precipitate. LCMS after 3 mins showed complete conversion. The mixture was diluted with additional dichloromethane (10 mL), and methanol was added followed by triethylamine. The mixture was evaporated to dryness, and the process was repeated one more time to obtain a crude product. The crude was dissolved in 1 mL of DMSO and purified via reverse phase column chromatography (0.1% formic acid and acetonitrile mixture) to obtain 3-cyano-1-(2,2-difluoroethyl)-4-(3-hydroxy-2,6-dimethyl-phenyl)pyrrolo[2,3-b]pyridine-6-carboxamide (30 mg, 50% yield). LCMS: m/z=371.1 [M+1]. ¹H NMR (400 MHz, DMSO) δ 9.24 (s, 1H), 8.59 (s, 1H), 8.50 (s, 1H), 7.70 (s, 1H), 7.60 (s, 1H), 6.91 (d, J=8.3 Hz, 1H), 6.75 (d, J=8.1 Hz, 1H), 6.71-6.37 (m, 1H), 4.94 (td, J=15.2, 3.7 Hz, 2H), 1.70 (s, 3H), 1.63 (s, 3H).

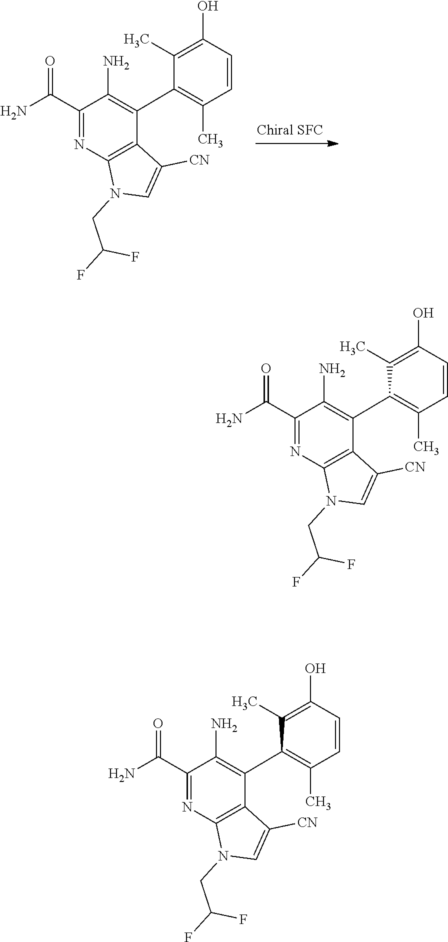

### Step 6: Chiral Separation

The racemate (410 mg, 1.1 mmol) was separated via chiral SFC to afford (P)-3-cyano-1-(2,2-difluoroethyl)-4-(3-hydroxy-2,6-dimethylphenyl)-1H-pyrrolo[2,3-b]pyridine-6-carboxamide (93). Column: DAICEL CHIRALPAK OJ, 30×150 mm, 5 μm; Conditions: Isocratic at 20% methanol with 80% CO₂: Flow Rate: 100 mL/min). LCMS m/z 371.10 (M+H⁺). ¹H NMR (400 MHz, DMSO) δ 9.25 (s, 1H), 8.59 (s, 1H), 8.51 (s, 1H), 7.71 (s, 1H), 7.61 (s, 1H), 6.91 (d, J=8.3 Hz, 1H), 6.75 (d, J=8.1 Hz, 1H), 6.54 (tt, J=54.8, 3.9 Hz, 1H), 4.94 (td, J=15.1, 3.9 Hz, 2H), 1.70 (s, 3H), 1.63 (s, 3H).

### Example 82. (P)-6-amino-2,3-bis(difluoromethyl)-7-(3-hydroxy-2,6-dimethyl-phenyl)benzimidazole-5-carboxamide (94)

### Step 1: 6-Amino-2-(difluoromethyl)-7-(3-methoxy-2,6-dimethyl-phenyl)-3H-benzimidazole-5-carbonitrile

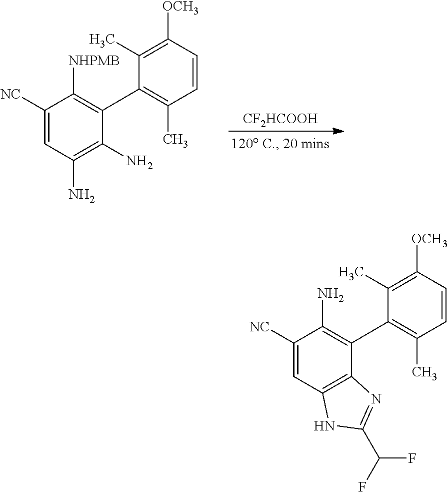

A solution of 4,5-diamino-3-(3-methoxy-2,6-dimethyl-phenyl)-2-[(4-methoxyphenyl)methylamino]benzonitrile (9.0 g, 22.4 mmol) in 2,2-difluoroacetic acid (25 mL) was stirred at 120° C. for 20 minutes. Upon completion of the reaction, the mixture was cooled to room temperature, and most of 2,2-difluoroacetic acid was removed. Then the mixture was poured into water (500 mL) and extracted with ethyl acetate (3×300 mL). The organic layer was washed with water and brine, dried with anhydrous sodium sulfate, and concentrated under reduced pressure. The residue was purified by column chromatography (0-20% EtOAc in hexane) to afford 6-amino-2-(difluoromethyl)-7-(3-methoxy-2,6-dimethyl-phenyl)-3H-benzimidazole-5-carbonitrile (7.1 g, 93% yield). LC-MS m/z: 343.20 [M+H]⁺.

### Step 2: Synthesis of 6-amino-2,3-bis(difluoromethyl)-7-(3-methoxy-2,6-dimethyl-phenyl)benzimidazole-5-carbonitrile

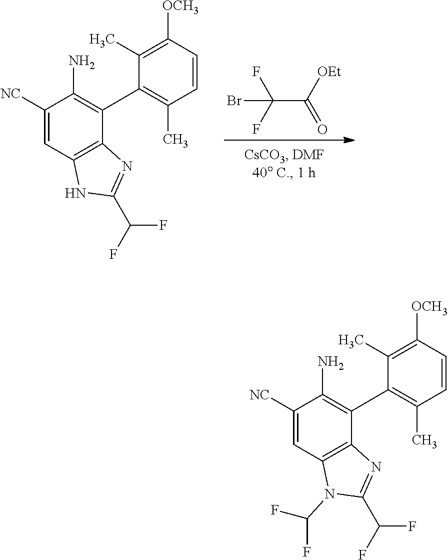

To a solution of 6-amino-2-(difluoromethyl)-7-(3-methoxy-2,6-dimethyl-phenyl)-3H-benzimidazole-5-carbonitrile (3.5 g, 10.2 mmol) in DMF (100 ml) was added cesium carbonate (6.7 g, 2.0 equiv., 20.4 mmol) at room temperature. Then ethyl 2-bromo-2,2-difluoro-acetate (4.2 g, 2.0 equiv., 20.4 mmol, 2.6 ml) was added to the mixture. The reaction was heated to 40° C., stirred for one hour until completion, cooled down to temperature, quenched with water (200 mL), and extracted with ethyl acetate (3×200 mL). The organic layer was washed with brine, dried with anhydrous sodium sulfate, and concentrated under reduced pressure. Afford the title compound 6-amino-2,3-bis(difluoromethyl)-7-(3-methoxy-2,6-dimethyl-phenyl)benzimidazole-5-carbonitrile (2.5 g, 62% yield) without purification and move to next step. LCMS: m/z=393.15. [M+H]⁺

### Step 3: Synthesis of 6-amino-2,3-bis(difluoromethyl)-7-(3-methoxy-2,6-dimethyl-phenyl)benzimidazole-5-carboxamide

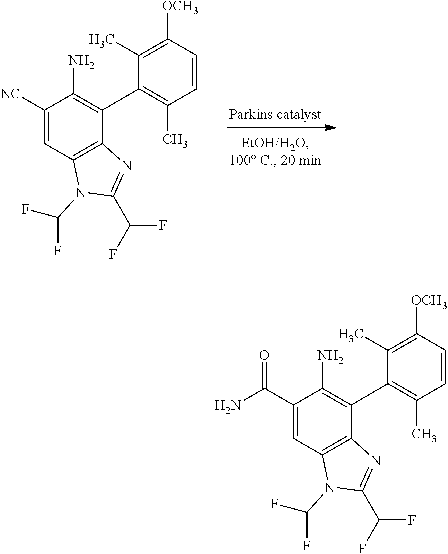 [SMILES: COc1ccc(C)c(-c2c(N)c(C#N)cc3c2nc(C(F)F)n3C(F)F)c1C ; COc1ccc(C)c(-c2c(N)c(C(N)=O)cc3c2nc(C(F)F)n3C(F)F)c1C]

To a solution of 6-amino-2,3-bis(difluoromethyl)-7-(3-methoxy-2,6-dimethyl-phenyl)benzimidazole-5-carbonitrile (2.5 g, 6.4 mmol) in EtOH (50 ml) and water (20 ml) was added Parkins catalyst (350 mg, 0.1 equiv., 0.64 mmol) at room temperature. Then the reaction was heated to 100° C. and stirred for 20 minutes until completion. The mixture was cooled to room temperature, and most of the solvent was removed. The remaining mixture was extracted with ethyl acetate. The organic layer was washed with brine, dried with anhydrous sodium sulfate, and concentrated under reduced pressure to afford the title compound 6-amino-2,3-bis(difluoromethyl)-7-(3-methoxy-2,6-dimethyl-phenyl)benz-imidazole-5-carboxamide (2.6 g, 99% yield) which was used without further purification. LCMS: m/z=411.10 [M+H]⁺.

### Step 4: Synthesis of 6-amino-2,3-bis(difluoromethyl)-7-(3-hydroxy-2,6-dimethyl-phenyl)benzimidazole-5-carboxamide

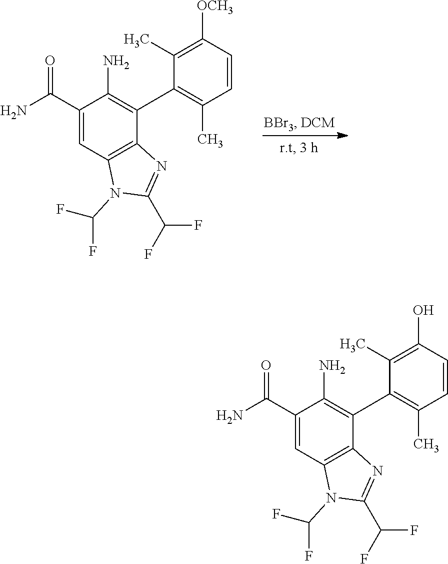 [SMILES: COc1ccc(C)c(-c2c(N)c(C(N)=O)cc3c2nc(C(F)F)n3C(F)F)c1C ; Cc1ccc(O)c(C)c1-c1c(N)c(C(N)=O)cc2c1nc(C(F)F)n2C(F)F]

Boron tribromide (19 mL, 3.0 equiv., 19 mmol) was added dropwise to a solution of 6-amino-2,3-bis(difluoromethyl)-7-(3-methoxy-2,6-dimethyl-phenyl)benzimidazole-5-carboxamide (2.6 g, 6.3 mmol) in dichloromethane (100 mL). The resulting mixture was stirred at room temperature for 3 hours, and then quenched with NaHCO₃ prior to extraction with two portions of ethyl acetate. The organic fraction was washed with brine and concentrated, and the resulting residue was purified by column chromatography (0-100% EtOAc in hexane) to afford the title compound 6-amino-2,3-bis(difluoromethyl)-7-(3-hydroxy-2,6-dimethyl-phenyl)benzimidazole-5-carboxamide (2.05 g, 82% yield). ¹H NMR (400 MHz, DMSO) δ 9.12 (s, 1H), 8.23-7.86 (m, 3H), 7.52-7.15 (m, 2H), 6.91 (d, J=8.2 Hz, 1H), 6.72 (d, J=8.2 Hz, 1H), 5.41 (s, 2H), 1.69 (s, 3H), 1.62 (s, 3H). ¹⁹F NMR (376 MHz, DMSO) δ −94.27, −115.68. LCMS: m/z=397.10 [M+H]⁺.

### Step 5: Synthesis of (P)-6-amino-2,3-bis(difluoromethyl)-7-(3-hydroxy-2,6-dimethyl-phenyl)benzimidazole-5-carboxamide

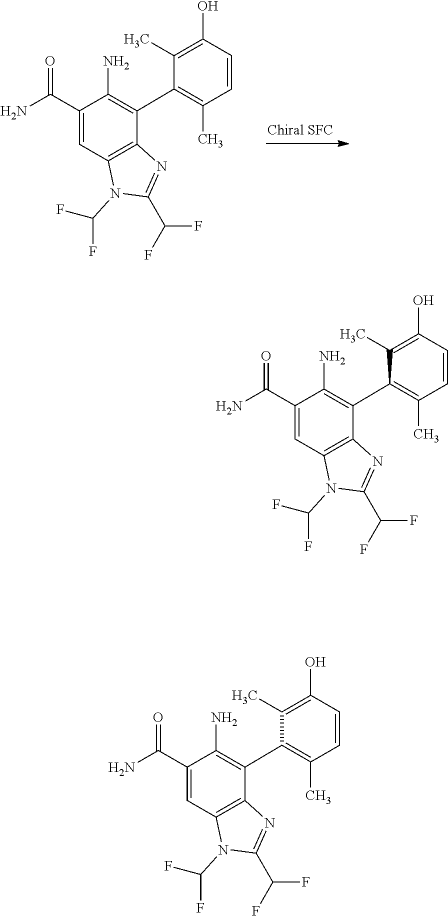

The racemic material (2.05 g, 5.17 mmol) was separated by chiral SFC, yielding (P)-6-amino-2,3-bis(difluoromethyl)-7-(3-hydroxy-2,6-dimethyl-phenyl)benzimidazole-5-carboxamide (94, 588 mg). Column: DAICEL CHIRALPAK OJ, 30×150 mm, 5 μm; Conditions: isocratic at 15% methanol with 85% CO₂; Flow Rate: 100 mL/min). ¹H NMR (400 MHz, DMSO) δ 9.19 (s, 1H), 8.16 (s, 1H), 8.12 (t, J=57.4 Hz, 1H), 8.01 (s, 1H), 7.48 (s, 1H), 7.40 (t, J=51.9 Hz, 1H), 6.98 (d, J=8.2 Hz, 1H), 6.80 (d, J=8.1 Hz, 1H), 5.48 (s, 2H), 1.76 (s, 3H), 1.70 (s, 3H). ¹⁹F NMR (376 MHz, DMSO) δ −94.26, −115.67. LCMS: m/z=397.10 [M+H]f.

### Example 83. (P)-6-amino-2-(difluoromethyl)-3-(3-fluoro-2-pyridyl)-7-(3-hydroxy-2,6-dimethyl-phenyl)benzimidazole-5-carboxamide (95)

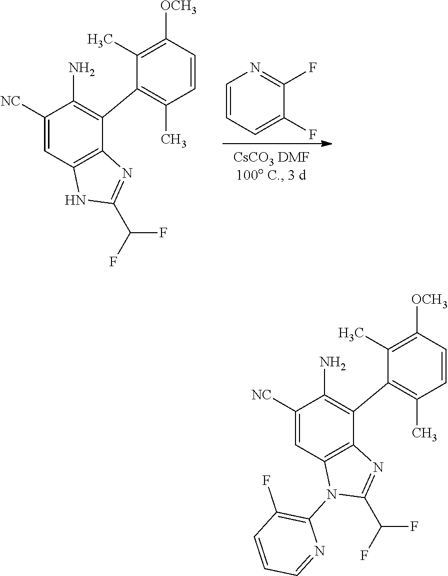

### Step 1: Synthesis of 6-amino-2-(difluoromethyl)-3-(3-fluoro-2-pyridyl)-7-(3-methoxy-2,6-dimethyl-phenyl)benzimidazole-5-carbonitrile

To a solution of 6-amino-2-(difluoromethyl)-7-(3-methoxy-2,6-dimethyl-phenyl)-3H-benzimidazole-5-carbonitrile (4.0 g, 11.7 mmol) in DMF (50 ml) was added cesium carbonate (7.6 g, 2.0 equiv., 23.4 mmol) at room temperature. Then 2,3-difluoropyridine (2.7 g, 2.0 equiv., 23.4 mmol, 2.12 ml) was added to the mixture. The reaction was heated to 100° C., stirred for 3 days until completion, cooled to room temperature, quenched with water (200 mL), and extracted with ethyl acetate (3×200 mL). The organic layer was washed with brine, dried with anhydrous sodium sulfate, and concentrated under reduced pressure. The residue was purified by column chromatography (0-100% EtOAc in hexane) to afford 6-amino-2-(difluoromethyl)-3-(3-fluoro-2-pyridyl)-7-(3-methoxy-2,6-dimethyl-phenyl)benzimidazole-5-carbonitrile (3.8 g, 74% yield) which was used without purification. LCMS: m/z=438.10 [M+H]⁺.

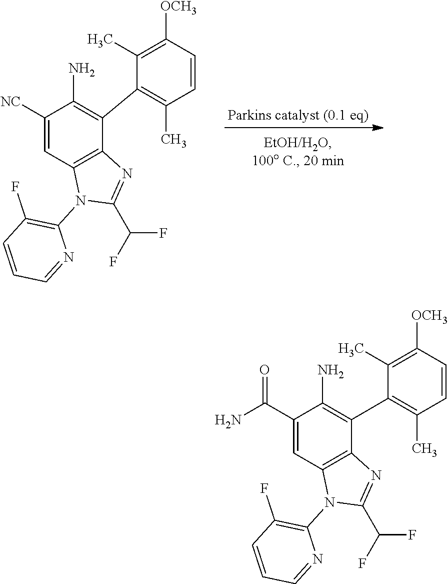 [SMILES: COc1ccc(C)c(-c2c(N)c(C#N)cc3c2nc(C(F)F)n3-c2ncccc2F)c1C ; COc1ccc(C)c(-c2c(N)c(C(N)=O)cc3c2nc(C(F)F)n3-c2ncccc2F)c1C]

### Step 2: Synthesis of 6-amino-2-(difluoromethyl)-3-(3-fluoro-2-pyridyl)-7-(3-methoxy-2,6-dimethyl-phenyl)benzimidazole-5-carboxamide

To a solution of 6-amino-2-(difluoromethyl)-3-(3-fluoro-2-pyridyl)-7-(3-methoxy-2,6-dimethyl-phenyl)benzimidazole-5-carbonitrile (3.8 g, 8.7 mmol) in EtOH (50 mL) and water (20 mL) was added Parkins catalyst (370 mg, 0.1 equiv., 0.84 mmol) at room temperature. The mixture was then heated to 100° C. and stirred for 20 minutes until completion. The mixture was cooled to room temperature, and most of the solvent was removed. The remaining solution was extracted with ethyl acetate. The organic layer was washed with brine, dried with anhydrous sodium sulfate, and concentrated under reduced pressure to afford 6-amino-2-(difluoromethyl)-3-(3-fluoro-2-pyridyl)-7-(3-methoxy-2,6-dimethyl-phenyl)benzimidazole-5-carboxamide (3.0 g, 76% yield) which was used without further purification. LCMS: m/z=456.15 [M+H]⁺

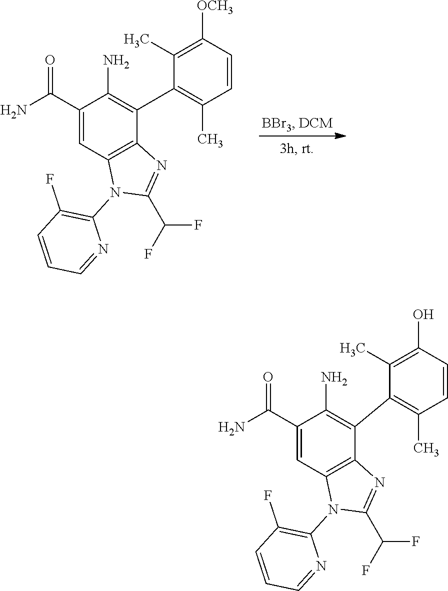 [SMILES: COc1ccc(C)c(-c2c(N)c(C(N)=O)cc3c2nc(C(F)F)n3-c2ncccc2F)c1C ; Cc1ccc(O)c(C)c1-c1c(N)c(C(N)=O)cc2c1nc(C(F)F)n2-c1ncccc1F]

### Step 3: Synthesis of 6-amino-2-(difluoromethyl)-3-(3-fluoro-2-pyridyl)-7-(3-hydroxy-2,6-dimethyl-phenyl)benzimidazole-5-carboxamide

Boron tribromide (20 mL, 3.0 equiv., 20 mmol) was added dropwise to a solution of 6-amino-2-(difluoromethyl)-3-(3-fluoro-2-pyridyl)-7-(3-methoxy-2,6-dimethyl-phenyl)benzimidazole-5-carboxamide (3.0 g, 6.6 mmol) in dichloromethane (100 mL) at room temperature. The resulting mixture was stirred at room temperature for 3 hours, subsequently quenched with NaHCO₃, and extracted with two portions of ethyl acetate. The organic fraction was washed with brine and concentrated. The resulting residue was purified by column chromatography (0-100% EtOAc in hexane) to afford 6-amino-2-(difluoromethyl)-3-(3-fluoro-2-pyridyl)-7-(3-hydroxy-2,6-dimethyl-phenyl)benzimidazole-5-carboxamide (1.7 g, 58% yield).

¹H NMR (400 MHz, DMSO) δ 9.11 (s, 1H), 8.51 (d, J=4.7 Hz, 1H), 8.14 (d, J=9.5 Hz, 1H), 7.80-7.73 (m, 1H), 7.54 (s, 1H), 7.27 (s, 1H), 7.14 (s, 1H), 6.94 (d, J=8.1 Hz, 1H), 6.74 (d, J=8.0 Hz, 1H), 6.49 (s, 1H), 5.47 (s, 2H), 1.74 (s, 3H), 1.68 (s, 3H). ¹⁹F NMR (376 MHz, DMSO) δ −115.21, −124.89. LCMS: m/z=442.05 [M+H]⁺.

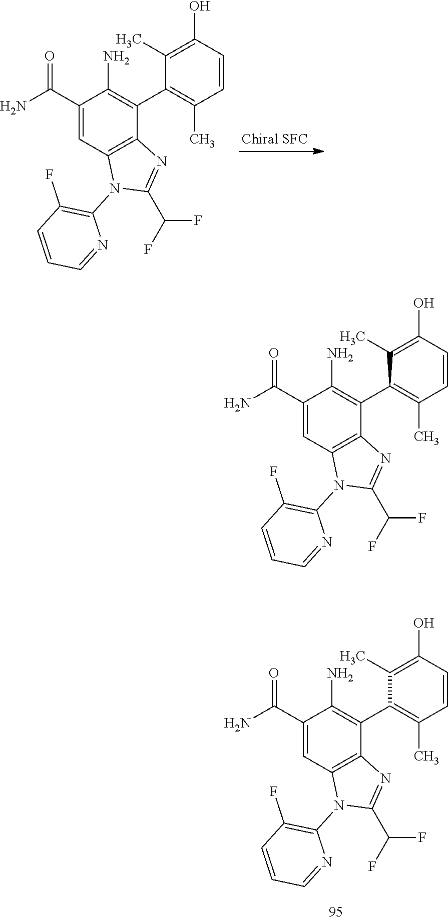

### Step 4: Chiral Separation

The racemic material (1.7 g, 3.9 mmol) was separated via chiral SFC. Column: DAICEL CHIRALPAK OJ, 30×150 mm, 5 μm; Conditions: isocratic at 20% methanol with 80% CO₂; Flow Rate: 100 mL/min) provided (P)-6-amino-2-(difluoromethyl)-3-(3-fluoro-2-pyridyl)-7-(3-hydroxy-2,6-dimethyl-phenyl)benzimidazole-5-carboxamide (95, 446 mg). ¹H NMR (400 MHz, DMSO) δ 9.12 (s, 1H), 8.51 (d, J=4.7 Hz, 1H), 8.15 (ddd, J=9.8, 8.4, 1.4 Hz, 1H), 7.89-7.85 (m, 1H), 7.76 (dt, J=8.4, 4.2 Hz, 1H), 7.54 (d, J=1.8 Hz, 1H), 7.14 (m, 2H), 6.94 (d, J=8.1 Hz, 1H), 6.74 (d, J=8.1 Hz, 1H), 5.47 (s, 2H), 1.74 (s, 3H), 1.68 (s, 3H). ¹⁹F NMR (376 MHz, DMSO) δ −116.15, −124.89. LCMS: m/z=442.20 [M+H]⁺

### Example 84. (M)-5-amino-1-ethyl-4-(3-hydroxy-2-methylphenyl)-1H-pyrrolo[2,3-b]pyridine-3,6-dicarboxamide (96)

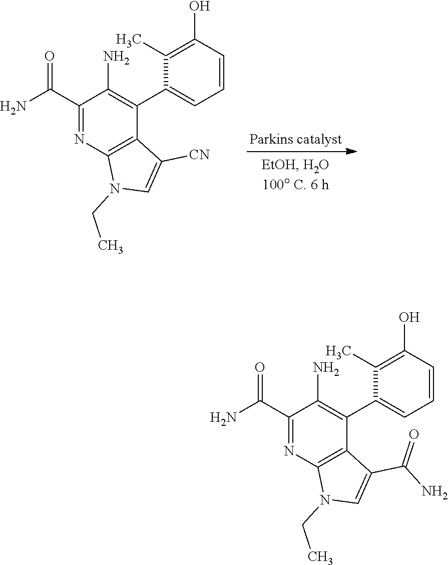

To a solution of (M)-5-amino-3-cyano-1-ethyl-4-(3-hydroxy-2-methyl-phenyl)pyrrolo[2,3-b]pyridine-6-carboxamide (61 mg, 0.18 mmol) in EtOH (5 ml) and water (2.5 ml) was added Parkins catalyst (15.6 mg, 0.2 equiv., 0.036 mmol) at room temperature. Then the reaction was heated to 100° C. and stirred for 6 hours until completion. The mixture was cooled to room temperature, and most of the solvent was removed. The remaining solution was extracted with ethyl acetate (3×20 mL). The organic layer was washed with water and brine, dried with anhydrous sodium sulfate, and concentrated under reduced pressure. The residue was purified by column chromatography (0-100% EtOAc in hexane) to afford (M)-5-amino-1-ethyl-4-(3-hydroxy-2-methyl-phenyl)pyrrolo[2,3-b]pyridine-3,6-dicarboxamide (96, 54 mg, 84% yield). ¹H NMR (400 MHz, DMSO) b 9.53 (s, 1H), 8.17 (d, J=3.1 Hz, 1H), 8.09 (s, 1H), 7.41 (d, J=3.1 Hz, 1H), 7.08 (t, J=7.8 Hz, 1H), 6.90-6.81 (m, 1H), 6.55 (dd, J=7.5, 1.2 Hz, 2H), 5.65 (s, 2H), 5.17 (s, 1H), 4.36-4.16 (m, 2H), 1.71 (s, 3H), 1.34 (t, J=7.2 Hz, 3H). LC-MS m/z [M+H]⁺=354.100.

### Example 85. (P)-1-ethyl-4-(3-hydroxy-2,6-dimethylphenyl)-3-(1H-imidazol-1-yl)-1H-pyrrolo[2,3-b]pyridine-6-carboxamide (97)

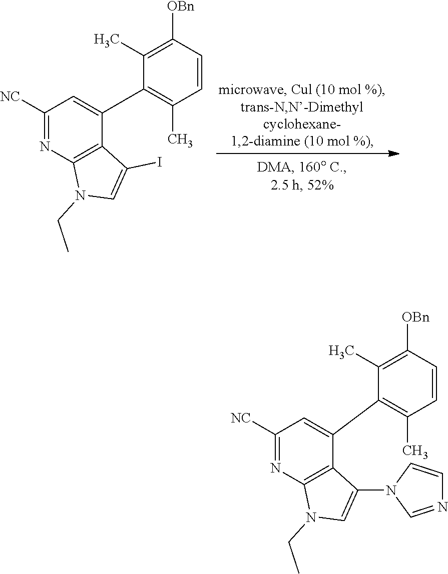 [SMILES: CCn1cc(I)c2c(-c3c(C)ccc(OCc4ccccc4)c3C)cc(C#N)nc21 ; CCn1cc(-n2ccnc2)c2c(-c3c(C)ccc(OCc4ccccc4)c3C)cc(C#N)nc21]

### Step 1: Synthesis of 4-(3-(benzyloxy)-2,6-dimethylphenyl)-1-ethyl-3-(1H-imidazol-1-yl)-1H-pyrrolo[2,3-b]pyridine-6-carbonitrile

A microwave vial containing intermediate 34-3 (500 mg, 0.99 mmol, 1.0 equiv), copper(I) iodide (18.8 mg, 0.10 mmol, 0.10 equiv), (1R,2R)—N1,N2-dimethylcyclohexane-1,2-diamine (15.5 μL, 0.098 mmol, 0.10 equiv). Cs₂CO₃ (674 mg, 2.07 mmol, 2.10 equiv) and imidazole (141 mg, 2.07 mmol, 2.10 equiv) was back-filled with N₂ twice before anhydrous DMA (7 mL) was added. The reaction was heated to 160° C. in a microwave reactor for 2.5 h. Upon completion, the mixture was diluted with EtOAc (100 mL) and washed with DI water (3×50 mL). The aqueous layer was back-extracted with EtOAc (2×50 mL) and the combined organic phases washed with brine (1×80 mL), dried (MgSO₄), filtered and concentrated in vacuo. Purification via automated flash column chromatography (80 g Redisep cartridge, 0-80% EtOAc-Hexanes, product eluted at 63% EtOAc) afforded 4-(3-benzyloxy-2,6-dimethyl-phenyl)-1-ethyl-3-imidazol-1-yl-pyrrolo[2,3-b]pyridine-6-carbonitrile (230 mg, 0.51 mmol, 52%). LCMS (ES, m/z), found [M]+H⁺: 448.2.

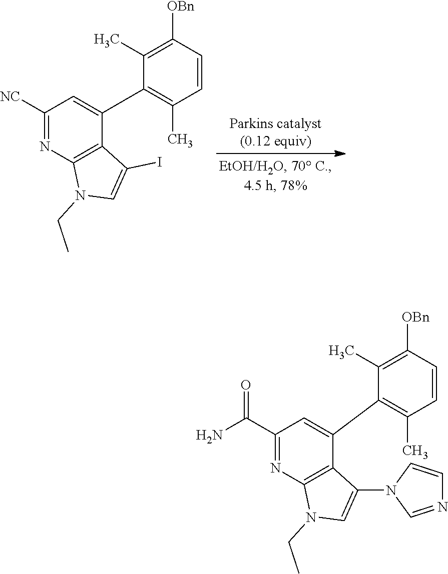 [SMILES: CCn1cc(I)c2c(-c3c(C)ccc(OCc4ccccc4)c3C)cc(C#N)nc21 ; CCn1cc(-n2ccnc2)c2c(-c3c(C)ccc(OCc4ccccc4)c3C)cc(C(N)=O)nc21]

### Step 2: Synthesis of 4-(3-(benzyloxy)-2,6-dimethylphenyl)-1-ethyl-3-(1H-imidazol-1-yl)-1H-pyrrolo[2,3-b]pyridine-6-carboxamide

4-(3-benzyloxy-2,6-dimethyl-phenyl)-1-ethyl-3-imidazol-1-yl-pyrrolo[2,3-b]pyridine-6-carbonitrile (228 mg, 0.51 mmol, 1.0 equiv) and Ghaffar-Parkins catalyst (26.2 mg, 0.06 mmol, 0.12 equiv) were suspended in EtOH (10 mL) and water (3.5 mL). The mixture was heated to 70° C. for 4.5 h. Upon completion, the reaction was concentrated in vacuo and the crude residue dissolved in EtOAc (30 mL) and washed with water (1×30 mL). The aq layer was back-extracted (2×35 mL) and the combined organic phases washed with brine (1×100 mL), dried (MgSO₄), filtered and concentrated in vacuo to afford 4-(3-benzyloxy-2,6-dimethyl-phenyl)-1-ethyl-3-imidazol-1-yl-pyrrolo[2,3-b]pyridine-6-carboxamide (183.7 mg, 0.39 mmol, 78%). LCMS (ES, m/z), found [M]+H⁺, 466.2.

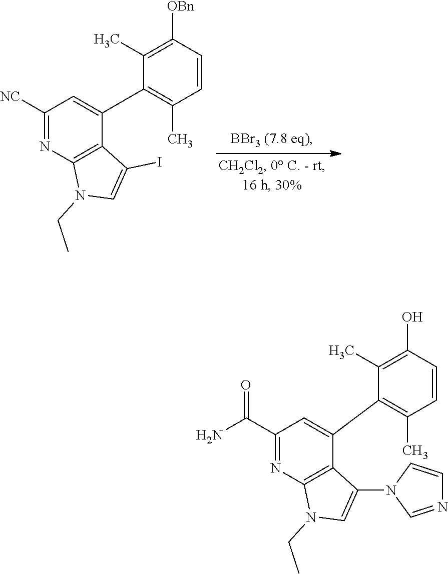 [SMILES: CCn1cc(I)c2c(-c3c(C)ccc(OCc4ccccc4)c3C)cc(C#N)nc21 ; CCn1cc(-n2ccnc2)c2c(-c3c(C)ccc(O)c3C)cc(C(N)=O)nc21]

### Step 3: Synthesis of 1-ethyl-4-(3-hydroxy-2,6-dimethylphenyl)-3-(1H-imidazol-1-yl)-1H-pyrrolo[2,3-b]pyridine-6-carboxamide

4-(3-benzyloxy-2,6-dimethyl-phenyl)-1-ethyl-3-imidazol-1-yl-pyrrolo[2,3-b]pyridine-6-carboxamide (248 mg, 0.53 mmol, 1.0 equiv) was suspended in CH₂Cl₂ (3 mL) under N2 and cooled to 0° C. BBr₃ (2.1 mL, 2.1 mmol, 3.9 equiv) was added dropwise and the reaction gradually warmed to rt. After 3 h, the reaction was cooled to 0° C. and a further portion of BBr₃ (2.1 mL, 2.1 mmol, 3.9 equiv) was added with additional CH₂Cl₂ (2 mL). The reaction was gradually warmed to rt and stirred overnight. Upon completion, the reaction was reverse quenched in ice-cold satd. NaHCO₃ solution and extracted with EtOAc (60 mL). The aq layer was back-extracted with EtOAc (2×30 mL) and the combined organic phases were washed with brine (1×150 mL), dried (MgSO₄), filtered and concentrated in vacuo to a crude residue. Purification via automated flash column chromatography (40 g redisep column, eluting with 0-10% MeOH—CH₂Cl₂, product eluted at 7% MeOH) afforded 1-ethyl-4-(3-hydroxy-2,6-dimethyl-phenyl)-3-imidazol-1-yl-pyrrolo[2,3-b]pyridine-6-carboxamide (59 mg, 0.16 mmol, 30%). LCMS (ES, m/z), found [M]+H⁺: 376.2.

### Step 4: Chiral Separation

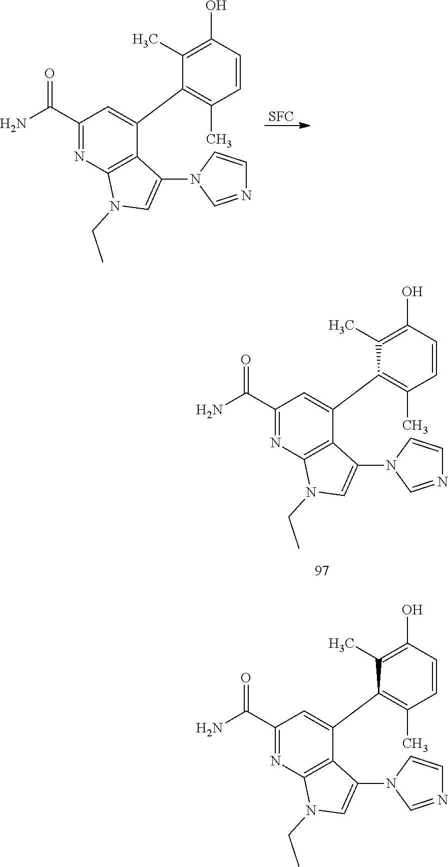

The racemate was purified by SFC to afford (P)-1-ethyl-4-(3-hydroxy-2,6-dimethylphenyl)-3-(1H-imidazol-1-yl)-1H-pyrrolo[2,3-b]pyridine-6-carboxamide (26.7 mg, 0.07 mmol, 8%, >98% purity). 1H NMR (400 MHz, DMSO) δ 9.41 (s, 1H), 8.41 (d, J 3.0 Hz, 1H), 8.18 (s, 1H), 7.78 (s, 1H), 7.74 (d, J=2.9 Hz, 1H), 7.67 (s, 1H), 7.27 (s, 1H), 7.06 (d, J=8.3 Hz, 1H), 6.89 (d, J 8.3 Hz, 1H), 6.28 (s, 1H), 4.47 (q. J 7.9 Hz, 1H), 1.90 (s, 3H), 1.83 (s, 3H), 1.24 (t, J 7.2 Hz, 3H). Exchangeable phenolic proton not observed. LCMS (ES, m/z), found [M]+H⁺: 376.2. Conditions: DAICEL CHIRALPAK AS (250 mm×30 mm, 10 μm); mobile phase: [CO₂-EtOH]; A %: 75%, B %:25%, isocratic elution mode).

### Example 86. (P)-5-amino-1-(difluoromethyl)-4-(3-hydroxy-2,6-dimethylphenyl)-1H-benzo[d][1,2,3]triazole-6-carboxamide (98)

### Step 1: Synthesis of 7-(3-methoxy-2,6-dimethylphenyl)-6-((4-methoxybenzyl)amino)-1H-benzo[d][1,2,3]triazole-5-carbonitrile

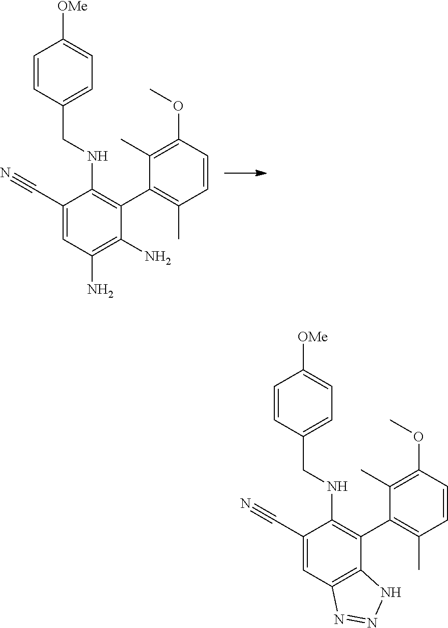 [SMILES: COc1ccc(CNc2c(C#N)cc(N)c(N)c2-c2c(C)ccc(OC)c2C)cc1 ; COc1ccc(CNc2c(C#N)cc3nn[nH]c3c2-c2c(C)ccc(OC)c2C)cc1]

The 5,6-diamino-3′-methoxy-2-((4-methoxybenzyl)amino)-2′,6′-dimethyl-[1,1′-biphenyl]-3-carbonitrile (1.70 g, 4.22 mmol) was dissolved in water (7.2 mL) and acetic acid (2.7 mL). The heterogeneous reaction mixture was cooled on ice. Sodium nitrite (379 mg, 5.49 mmol) in water (6 mL) was added dropwise via addition funnel at 0° C. for 10-30 min. The reaction mixture was warmed up to room temperature and stirred overnight. The resulting precipitate was collected by filtration after washing with water. The material was dried under high vacuum to provide 7-(3-methoxy-2,6-dimethylphenyl)-6-((4-methoxybenzyl)amino)-1H-benzo[d][1,2,3]triazole-5-carbonitrile (970 mg, 55.4%). LCMS (m/z): 414.30 [M+H]

### Step 2: Synthesis of 1-(difluoromethyl)-4-(3-methoxy-2,6-dimethylphenyl)-5-((4-methoxybenzyl)amino)-1H-benzo[d][1,2,3]triazole-6-carbonitrile

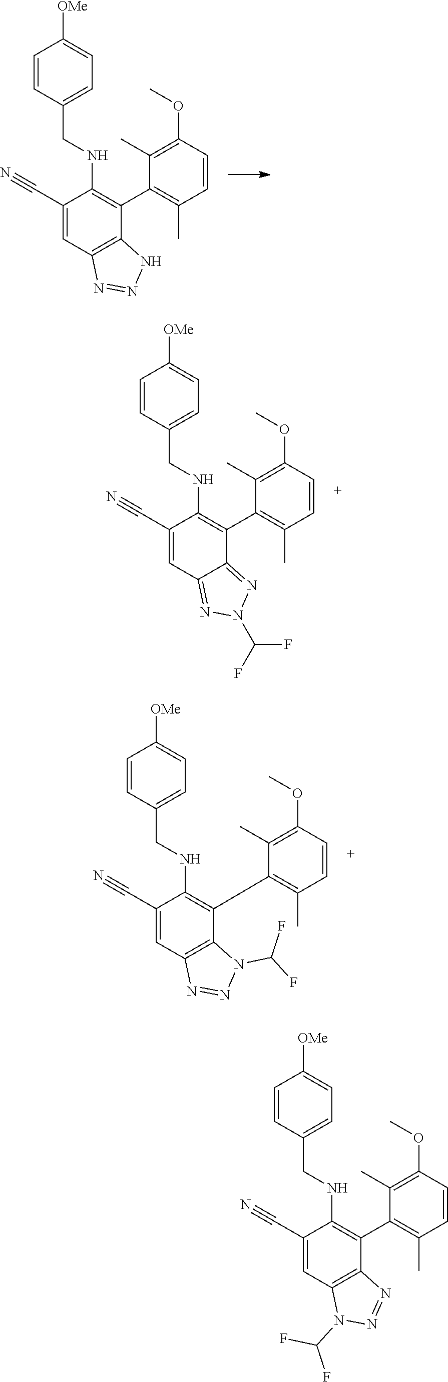 [SMILES: COc1ccc(CNc2c(C#N)cc3nn[nH]c3c2-c2c(C)ccc(OC)c2C)cc1 ; COc1ccc(CNc2c(C#N)cc3nn(C(F)F)nc3c2-c2c(C)ccc(OC)c2C)cc1 ; COc1ccc(CNc2c(C#N)cc3nnn(C(F)F)c3c2-c2c(C)ccc(OC)c2C)cc1 ; COc1ccc(CNc2c(C#N)cc3c(nnn3C(F)F)c2-c2c(C)ccc(OC)c2C)cc1]

To a solution of 7-(3-methoxy-2,6-dimethylphenyl)-6-((4-methoxybenzyl)amino)-1H-benzo[d][1,2,3]triazole-5-carbonitrile (360 mg, 0.870 mmol) in DMF (8.7 mL, 0.1 M) was added cesium carbonate (567 mg, 1.741 mmol), ethyl 2-bromo-2,2-difluoro-acetate (353.4 mg, 1.741 mmol) at room temperature. The reaction mixture was heated to 100° C. for 2 h and quenched by addition of water. The mixture was partitioned with ethyl acetate and the aqueous layer was washed with ethyl acetate (3×30 mL). The combined organic layers were washed with brine and dried over magnesium sulfate. The mixture was filtered, rinsed with EtOAc, and the solvent removed. The residue was purified via flash column chromatography to give three products, 2-(difluoromethyl)-7-(3-methoxy-2,6-dimethylphenyl)-6-((4-methoxybenzyl)amino)-2H-benzo[d][1,2,3]triazole-5-carbonitrile (A, 160.9 mg, 39.8%), 1-(difluoromethyl)-7-(3-methoxy-2,6-dimethylphenyl)-6-((4-methoxybenzyl)amino)-1H-benzo[d][1,2,3]triazole-5-carbonitrile (B, 54.8 mg, 13.6%), 1-(difluoromethyl)-4-(3-methoxy-2,6-dimethylphenyl)-5-((4-methoxybenzyl)amino)-1H-benzo[d][1,2,3]triazole-6-carbonitrile (C, 117.8 mg, 29.1%) LCMS (m/z): 464.30 [M+H]

### Step 3: Synthesis of 1-(difluoromethyl)-4-(3-methoxy-2,6-dimethylphenyl)-5-((4-methoxybenzyl)amino)-1H-benzo[d][1,2,3]triazole-6-carboxamide

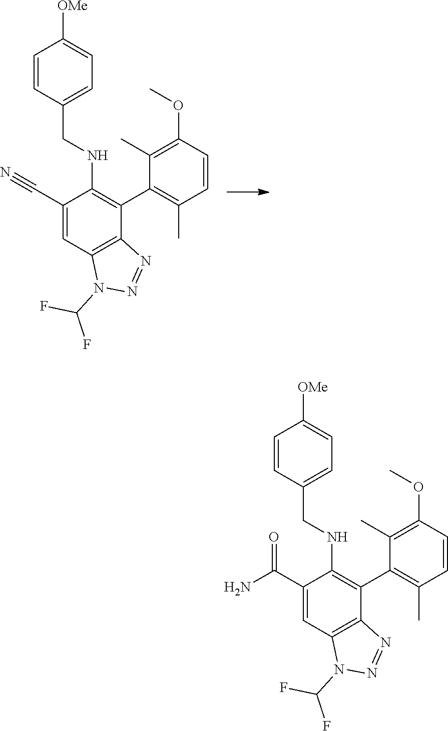 [SMILES: COc1ccc(CNc2c(C#N)cc3c(nnn3C(F)F)c2-c2c(C)ccc(OC)c2C)cc1 ; COc1ccc(CNc2c(C(N)=O)cc3c(nnn3C(F)F)c2-c2c(C)ccc(OC)c2C)cc1]

A solution of 1-(difluoromethyl)-4-(3-methoxy-2,6-dimethylphenyl)-5-((4-methoxybenzyl)amino)-1H-benzo[d][1,2,3]triazole-6-carbonitrile (116 mg, 0.250 mmol) in DMSO (2.5 mL, 0.1 M) was treated with lithium hydroxide (0.31 mL, 2 M) and hydrogen peroxide (30%, 56.8 mg, 0.05 mL). The mixture was stirred at room temperature for 1 h. The mixture was diluted with water (10 mL) and extracted with ethyl acetate (3×10 mL). The organic layers were combined, dried over MgSO₄, filtered, and concentrated in vacuo. The residue was moved to the next reaction without further purification. 1-(difluoromethyl)-4-(3-methoxy-2,6-dimethylphenyl)-5-((4-methoxybenzyl)amino)-1H-benzo[d][1,2,3]triazole-6-carboxamide. LCMS (m/z): 482.10 [M+H]

### Step 4: Synthesis of 5-amino-1-(difluoromethyl)-4-(3-methoxy-2,6-dimethylphenyl)-1H-benzo[d][1,2,3]triazole-6-carboxamide

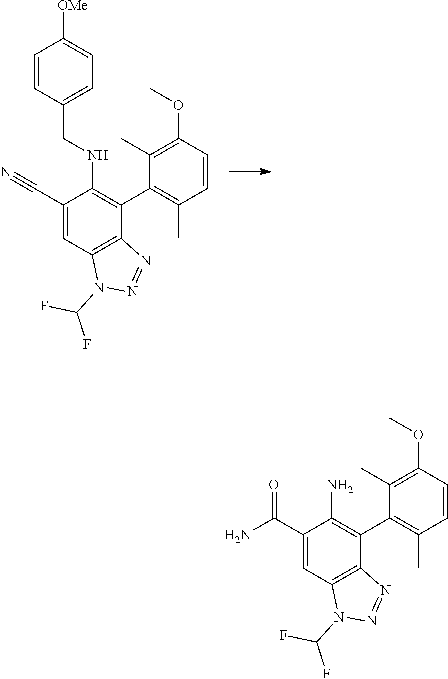 [SMILES: COc1ccc(CNc2c(C#N)cc3c(nnn3C(F)F)c2-c2c(C)ccc(OC)c2C)cc1 ; COc1ccc(C)c(-c2c(N)c(C(N)=O)cc3c2nnn3C(F)F)c1C]

To a solution of 1-(difluoromethyl)-4-(3-methoxy-2,6-dimethylphenyl)-5-((4-methoxybenzyl)amino)-1H-benzo[d][1,2,3]triazole-6-carboxamide (116 mg, 0.240 mmol) in DCM (4 mL) was added 2,2,2-trifluoroacetic acid (1 mL), kept under room temperature for 6 h. The reaction mixture was quenched with water, washed with DCM. The organic layers were combined and dried over MgSO₄. After filtration, the organic layer was concentrated and purified by column chromatography to give 5-amino-1-(difluoromethyl)-4-(3-methoxy-2,6-dimethylphenyl)-1H-benzo[d][1,2,3]triazole-6-carboxamide (66.7 mg, 76.5%). LCMS (m/z): 362.30 [M+H].

### Step 5: Synthesis of 5-amino-1-(difluoromethyl)-4-(3-hydroxy-2,6-dimethylphenyl)-1H-benzo[d][1,2,3]triazole-6-carboxamide

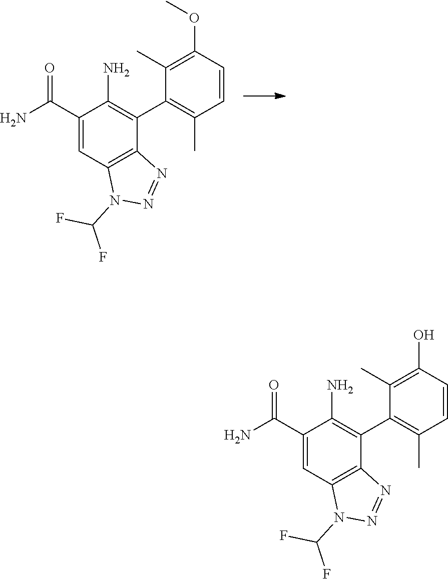 [SMILES: COc1ccc(C)c(-c2c(N)c(C(N)=O)cc3c2nnn3C(F)F)c1C ; Cc1ccc(O)c(C)c1-c1c(N)c(C(N)=O)cc2c1nnn2C(F)F]

To a solution of 5-amino-1-(difluoromethyl)-4-(3-methoxy-2,6-dimethylphenyl)-1H-benzo[d][1,2,3]triazole-6-carboxamide (102 mg, 0.293 mmol) in DCM (5 mL) was added dropwise tribromoborane (5 mL, 1.468 mmol) at 0° C. The reaction mixture was warmed to room temperature for 1 h and quenched by water at 0° C. and basified with aq.NaHCO₃. The mixture was partitioned with DCM (3×10 mL), dried over MgSO₄, filtered, and concentrated in vacuo. The residue was purified by preparative reverse HPLC to give 5-amino-1-(difluoromethyl)-4-(3-hydroxy-2,6-dimethylphenyl)-1H-benzo[d][1,2,3]triazole-6-carboxamide (51.9 mg, 50.8%). LCMS (m/z): 348.10.

### Step 6: Chiral Separation

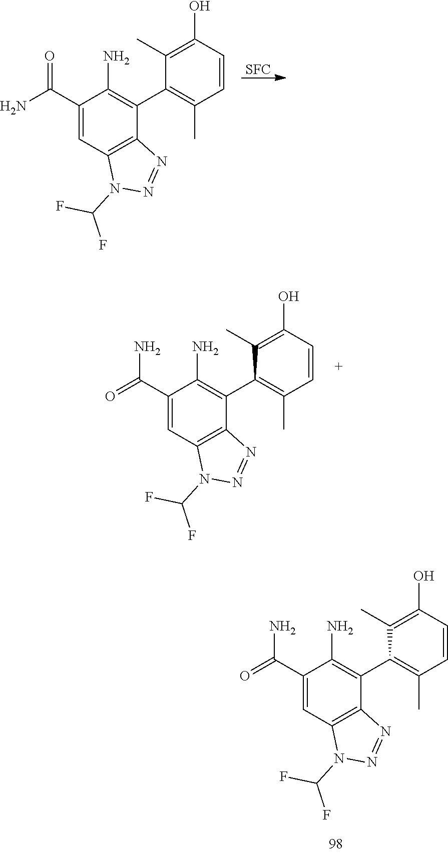

The racemic material was separated via chiral SFC (Column: DAICEL CHIRALPAK OJ, 30×150 mm, 5 μm; Conditions: isocratic at 80% CO₂ with 20% methanol; Flow Rate: 100 mL/min) to provide (P)-5-amino-1-(difluoromethyl)-4-(3-hydroxy-2,6-dimethylphenyl)-1H-benzo[d][1,2,3]triazole-6-carboxamide (16.4 mg). LC-MS m/z 348.15 [M+H]; ¹H NMR (400 MHz, DMSO) δ 9.21 (s, 1H), 8.48 (t, J=57.6 Hz, 1H), 8.27 (s, 1H), 8.03 (s, 1H), 7.65 (s, 1H), 6.96 (d, J=8.2 Hz, 1H), 6.77 (d, J=8.2 Hz, 1H), 5.42 (s, 2H), 1.67 (d, J=25.4 Hz, 6H).

### Biological Examples

### Example 87. PKMYT1 Inhibitory Activity

Biochemical IC₅₀ values were determined via ADP-Glo™ assay. Cellular IC₅₀ values were determined via AlphaLISA assay.

ADP-Glo™ PKMYT1 ATP-depletion assay. The activity of PKMYT1 is measured using a luciferase-luciferin based ATP detection reaction to quantify ATP depletion resulting from kinase-catalyzed phosphoryl transfer to CDK1 or autophosphorylation of PKMYT1. The ADP generated as a byproduct of the reaction is converted to ATP and used to drive a luciferase reaction. Test compounds were dissolved in DMSO and dispensed into Corning 3824 384-well plates prior to the experiment such that the final DMSO concentration was <10%. To the compounds was added 3 μL of PKMYT1 (93 kD, full length, N-terminal GST tag from Signal Chem). The reaction was started by the addition of 2 μL of CDK1 (58 kDa, full length, N-terminal GST tag from Signal Chem) and ATP in assay buffer (50 mM Hepes, 40 mM NaCl, 10 mM MgCl₂, 1 mM DTT, 0.1 mg/mL BSA, 2.5 mM MnCl2). Final assay concentrations were 50 nM PKMYT1, 13.3 nM CDK1, and 88 μM ATP. Plates were centrifuged for 1 minute at 1000 RPM and incubated at RT for 120 minutes. After the incubation period, ADP-Glo™ reagent was added and the plates were incubated for another 40 minutes before finally adding the kinase detection reagent, 30 minutes after the addition, plates were placed on a PHERAstar® plate reader and read using the Luminescence Protocol, integration time at 0.5 seconds. Compounds exemplified above were tested by the PKMYT1 ATP depletion assay and exhibited IC₅₀ values as summarized in Table 2.

Cell lysate AlphaLISA® assay measuring inhibition of phosphorvlation of CDK1 Thr14. Inhibition of phosphorylation of CDK1 at Thr14 was determined using AlphaLISAn SureFire® Ultra™ Kit from Perkin Elmer (ALSU-PCDK1-A10K). HCC1806 cells were seeded at 2K cells per well (50 μl/well) in 384-well plates (Corning 3570) and stored overnight at 37° C. and 5% CO₂. The following day, the compounds dissolved in DMSO were introduced to the cells and incubated for 4 hours at 37° C. and 5% CO₂. The cells were then washed with PBS using Blue® Washer and 10 μl of lysis buffer containing protease inhibitor was added. The plate was placed on an orbital shaker (at 4° C.) for 30 minutes and stored overnight at −80° C. for complete lysing. The next day, the plates were thawed at room temperature for at least 30 min. Reagents were prepared in a dimmed light setting (<100 lumens). The assay was run by first adding 5 μl of the acceptor mix (reaction buffer 1: reaction buffer 2:activation buffer:acceptor beads 47:47:4:2), covering the plate with foil, shaking for 2 minutes, and incubating at RT for 60 minutes. The donor mix (dilution buffer:donor beads 47:3) was then added at 5 μl per well, placed on the shaker for 2 minutes and incubated at RT for 60 minutes before reading the plates on a PHERAstar® plate reader. The results were normalized to DMSO (0% Inhibition) and 3 μM of reference compound. Test compounds exhibited cellular IC₅₀ values as summarized in Table 2.

TABLE 2
Activity data for selected compounds of the invention

| Compound | Biochemical | Cellular |
|---|---|---|
| No. | IC₅₀ ^{a} | IC₅₀ ^{b} |
| 1 | +++++ | †††† |
| 2 | +++++ | ††††† |
| 3 | ++ | † |
| 4 | +++++ | ††††† |
| 5 | +++++ | ††††† |
| 6 | +++ | †††† |
| 7 | +++++ | †††† |
| 8 | +++++ | †††† |
| 9 | +++++ | †††† |
| 10 | ++++ | †† |
| 11 | +++++ | ††††† |
| 12 | +++++ | †††† |
| 13 | +++++ | ††††† |
| 14 | +++++ | ††††† |
| 15 | +++++ | ††† |
| 16 | ++ | † |
| 17 | ++ | † |
| 18 | ++ | † |
| 19 | + | † |
| 20 | + | † |
| 21 | +++ | † |
| 22 | +++++ | †††† |
| 23 | +++++ | ††††† |
| 24 | ++++ | †† |
| 25 | + | † |
| 26 | +++++ | †††† |
| 27 | +++++ | ††††† |
| 28 | ++++ | ††† |
| 29 | +++++ | ††††† |
| 30 | ++++ | †† |
| 31 | + | † |
| 32 | ++++ | †† |
| 33 | ++ | † |
| 34 | +++++ | †††† |
| 35 | ++ | † |
| 36 | ++ | † |
| 37 | ++ | †† |
| 38 | + | †† |
| 39 | + | †† |
| 40 | + | † |
| 41 | +++ | †† |
| 42 | ++ | †† |
| 43 | +++ | † |
| 44 | ++++ | † |
| 45 | + | † |
| 46 | ++++ | †† |
| 47 | ++++ | † |
| 48 | ++++ | † |
| 49 | ++++ | † |
| 50 | ++++ | ††† |
| 51 | +++ | † |
| 52 | ++++ | † |
| 53 | + | † |
| 54 | +++++ | †††† |
| 55 | +++++ | †††† |
| 56 | ++++ | † |
| 57 | ++++ | †††† |
| 58 | +++++ | †††† |
| 59 | ++ | † |
| 66 | +++++ | †††† |
| 67 | +++++ | ††††† |
| 68 | +++++ | ††††† |
| 69 | +++++ | ††††† |
| 70 | +++++ | ††††† |
| 71 | +++++ | ††††† |
| 72 | +++++ | ††††† |
| 73 | +++++ | ††††† |
| 74 | +++++ | ††††† |
| 75 | +++++ | ††††† |
| 76 | +++++ | ††††† |
| 77 | +++++ | ††††† |
| 78 | +++++ | ††††† |
| 79 | +++++ | ††††† |
| 81 | +++++ | ††††† |
| 82 | +++++ | ††††† |
| 83 | +++++ | ††††† |
| 84 | +++++ | ††††† |
| 85 | +++++ | ††††† |
| 86 | +++++ | ††††† |
| 87 | +++++ | ††††† |
| 88 | +++++ | ††††† |
| 89 | +++++ | ††††† |
| 90 | +++++ | ††††† |
| 91 | +++++ | ††††† |
| 92 | +++++ | ††††† |
| 93 | +++++ | ††††† |
| 94 | +++++ | ††††† |
| 95 | +++++ | ††††† |
| 96 | +++++ | ††††† |
| 97 | ++++ | †† |
| 98 | +++++ | ††††† |

^{a} +++++: IC₅₀ < 50 nM
++++: 50 nM ≤ IC₅₀ < 500 nM
+++: 500 nM ≤ IC₅₀ < 1 μM
++: 1 μM ≤ IC₅₀ < 5 μM
+: 5 μM ≤ IC₅₀
^{b} †††††: IC₅₀ < 50 nM
††††: 50 nM ≤ IC₅₀ < 500 nM
†††: 500 nM ≤ IC₅₀ < 1 μM
††: 1 μM ≤ IC₅₀ < 5 μM
†: 5 μM ≤ IC₅₀

### Example 88. Hepatocyte Stability Assay

Metabolic stability of testing compounds can be evaluated using human, rat, mouse, or other animal hepatocytes to assess intrinsic clearance.

Cryopreserved hepatocytes were removed from the liquid nitrogen tank and thawed in a 37° C. water bath. As soon as the cells pulled away from the vial wall, they were decanted into 48 mL of warm HT medium. Cells were centrifuged for four minutes at 420 rpm (50 g). After removing the supernatant, pellet was re-suspended in warm DMEM medium. Cell density was counted by a hemacytometer.

The assay was carried out in 96-well microtiter plates. Compounds were incubated for 0, 60, 120, and 180 minutes at 37° C. with hepatocytes. The reaction mixtures (50 μL) contained a final concentration of 1 μM test compound, 0.5 million cells/mL hepatocytes in the DMEM medium. At each of the time points (for example, 0, 1, 2, and 3 hours), 200 μL of quench solution (100% acetonitrile with 0.1% formic acid) with internal standard was transferred to each well. Midazolam was included as a positive control to verify assay performance. Plates were sealed and centrifuged at 4° C. for 15 minutes at 4000 rpm. The supernatant was transferred to fresh plates for LC/MS/MS analysis.

All samples were analyzed on LC/MS/MS using an AB Sciex API 4000 instrument, coupled to a Shimadzu LC-20AD LC Pump system. Analytical samples were separated using a Waters Atlantis T3 dC18 reverse phase HPLC column (20 mm×2.1 mm) at a flow rate of 0.5 mL/min. The mobile phase was 0.1% formic acid in water (solvent A) and 0.1% formic acid in 100% acetonitrile (solvent B).

The extent of metabolism was calculated as the disappearance of the test compound, compared to the 0-min control reaction incubations. Initial rates were calculated for the compound concentration and used to determine t_{1/2} values and subsequently, the intrinsic clearance, CLint=(0.693)(1/t_{1/2} (min))(mL incubation/million cells).

The testing results showed that certain compounds of the present disclosure have low rates of metabolic clearance in human hepatocytes.

### Example 89. Other Kinase Inhibitory Activity

Kinome profiles for test compounds were determined using a panel of 378 assays (374 kinases) all run in a radiometric format using 33P-labeled ATP (full human WT kinase [Km ATP]KinaseProfiler LeadHunter Panel; Eurofins). Each assay was run using a Mg/ATP mixture such that the concentration of ATP was the Km for ATP in that kinase. Details for example assays are provided below.

c-Src Inhibition Assay. c-Src (h) was incubated with 8 mM MOPS pH 7.0, 0.2 mM EDTA, 250 μM KVEKIGEGTYGVVYK (Cdc2 peptide), 10 mM magnesium acetate and [γ-³³P]-ATP (specific activity and concentration as required). The reaction was initiated by the addition of the Mg/ATP mixture. After incubation for 40 minutes at room temperature, the reaction was stopped by the addition of phosphoric acid to a concentration of 0.5%. An aliquot of the reaction was then spotted onto a filter and washed four times for four minutes in 0.425% phosphoric acid and once in methanol prior to drying and scintillation counting.

Lck Inhibition Assay. Lck (h) was incubated with 50 mM Tris pH 7.5, 0.1 mM EGTA, 0.1 mM Na₃VO₄, 250 μM KVEKIGEGTYGVVYK (Cdc2 peptide), 10 mM magnesium acetate and [γ-³³P]-ATP (specific activity and concentration as required). The reaction was initiated by the addition of the Mg/ATP mixture. After incubation for 40 minutes at room temperature, the reaction was stopped by the addition of phosphoric acid to a concentration of 0.5%. An aliquot of the reaction was then spotted onto a filter and washed four times for four minutes in 0.425% phosphoric acid and once in methanol prior to drying and scintillation counting.

A high selectivity for inhibition of PKMYT1 over other kinases was observed for certain compounds of the present disclosure.

### OTHER EMBODIMENTS

The foregoing disclosure has been described in some detail by way of illustration and example, for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive.

The scope of the invention should, therefore, be determined not with reference to the above description, but should instead be determined with reference to the following appended claims, along with the full scope of equivalents to which such claims are entitled.

1. A compound according to Formula I: 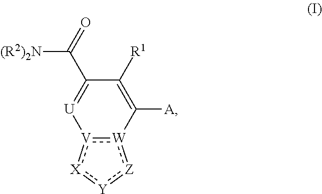
or a pharmaceutically acceptable salt thereof,
wherein:
each [symbol] represents a single bond, a double bond, or a delocalized π bond;
U is selected from the group consisting of N and CH;
V is selected from the group consisting of C and N;
W is selected from the group consisting of C and N;
X is selected from the group consisting of N, NR^{3a}, CR³, S, and O;
Y is selected from the group consisting of CR⁴, N, NR^{4a}, S, and O;
Z is selected from the group consisting of CR⁵, N, NR^{5a}, S, and O;
ring A is
3- to 14-membered carbocyclyl substituted with 1, 2, 3, 4, or 5 R⁶, or
5- to 14-membered heterocyclyl comprising at least one oxygen atom, nitrogen atom, or sulfur atom, wherein the heterocyclyl is substituted with 0, 1, 2, 3, or 4 R⁶;
R¹ is selected from the group consisting of amino, hydroxy, and hydrogen;
each R² is independently selected from the group consisting of hydrogen and C₁₋₆ alkyl;
R³, R⁴, and R⁵ are independently selected from the group consisting of hydrogen, amino, hydroxy, halogen, cyano, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, —NO₂, —CO(C₁₋₆ alkyl), —COOH, —COO(C₁₋₆ alkyl), —CON(R⁷)₂, -L-(5- to 12-membered heterocyclyl), -L-(5- to 14-membered heteroaryl), and -L-(C₆₋₁₄ aryl), wherein the 5- to 12-membered heterocyclyl, 5- to 14-membered heteroaryl, and C₆₋₁₄ aryl are independently substituted with 0, 1, 2, 3, or 4 R⁶;
each L is independently selected from the group consisting of a covalent bond, —O—, C₁₋₆ alkylene, and NR^{a};
any two adjacent R³, R⁴, and R⁵ are optionally taken together with the carbon atoms to which they are attached to form a 5- or 6-membered carbocyle or heterocycle, each of which is optionally substituted with 0, 1, 2, 3, or 4 R⁶;
R^{3a}, R^{4a}, R^{5a}, and R⁷ are independently selected from the group consisting of hydrogen, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, -L-(5- to 12-membered heterocyclyl), -L-(5- to 14-membered heteroaryl, and -L-(C₆₋₁₄ aryl), wherein the C₃₋₈ cycloalkyl, 5- to 12-membered heterocyclyl, 5- to 14-membered heteroaryl, and C₆₋₁₄ aryl are independently substituted with 0, 1, 2, 3, or 4 R⁶;
each R⁶ is independently selected from the group consisting of hydroxy, amino, cyano, halogen, C₁₋₆ alkyl, C₁₋₆ haloalkyl, C₁₋₆ hydroxyalkyl, C₁₋₆ alkoxy, oxo, —NO₂, —CHO, —CO(C₁₋₆ alkyl), —COOR^{a}, —CON(R^{a})₂, —SO₂(C₁₋₆ alkyl), and —SO₂N(R^{a})₂; and
each R^{a} is independently selected from the group consisting of hydrogen, C₁₋₆ alkyl, and C₁₋₆ haloalkyl;
provided that ring A is substituted with at least one R⁶ that is selected from the group consisting of hydroxy, amino, cyano, C₁₋₆ alkyl, C₁₋₆ haloalkyl, C₁₋₆ hydroxyalkyl, oxo, —NO₂, —CHO, —CO(C₁₋₆ alkyl), —COOR^{a}, —CON(R^{a})₂, and —SO₂N(R^{a})₂ when ring A is a carbocycle and R¹ and each R² are hydrogen; and
provided that ring A is substituted with at least one R⁶ that is selected from the group consisting of hydroxy, amino, cyano, halogen, C₁₋₆ haloalkyl, C₁₋₆ hydroxyalkyl, oxo, —NO₂, —CHO, —CO(C₁₋₆ alkyl), —COOR^{a}, —CON(R^{a})₂, —SO₂(C₁₋₆ alkyl), and —SO₂N(R^{a})₂ when ring A is a heterocycle and R¹ and each R² are hydrogen.

2. The compound of claim 1, or a pharmaceutically acceptable salt thereof, wherein R¹ is amino.

3. The compound of claim 1, or a pharmaceutically acceptable salt thereof, wherein R¹ is hydrogen.

4. The compound of any one of claims 1-3, or a pharmaceutically acceptable salt thereof, wherein each R² is hydrogen.

5. The compound of any one of claims 1-4, or a pharmaceutically acceptable salt thereof, wherein U is N, V is C, and W is C.

6. The compound of any one of claims 1-5, having a structure according to Formula Ia: 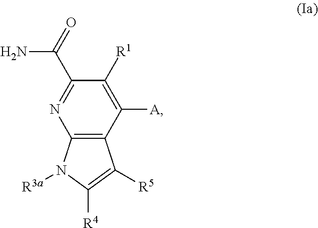
or a pharmaceutically acceptable salt thereof.

7. The compound of any one of claims 1-5, having a structure according to Formula Ib: 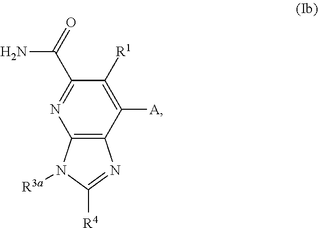
or a pharmaceutically acceptable salt thereof.

8. The compound of any one of claims 1-5, having a structure according to Formula Ic: 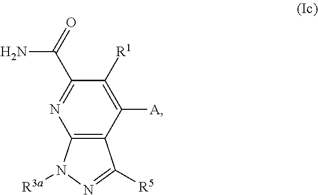
or a pharmaceutically acceptable salt thereof.

9. The compound of any one of claims 1-5, having a structure according to Formula Id: 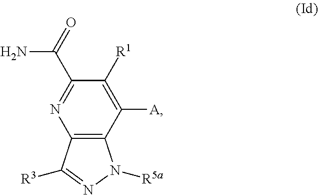
or a pharmaceutically acceptable salt thereof.

10. The compound of any one of claims 1-4, or a pharmaceutically acceptable salt thereof, wherein U is C, V is C, and W is C.

11. The compound of any one of claims 1-4 and 10, having a structure according to Formula IIa 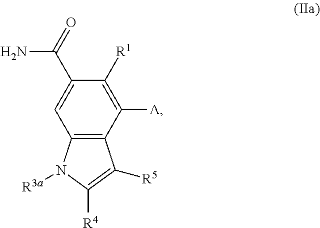
or a pharmaceutically acceptable salt thereof.

12. The compound of any one of claims 1-4 and 10, having a structure according to Formula IIb 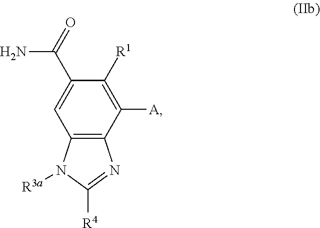
or a pharmaceutically acceptable salt thereof.

13. The compound of any one of claims 1-4 and 10, having a structure according to Formula IIc 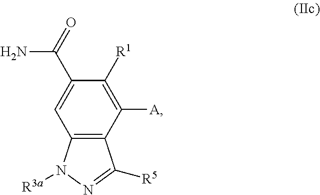
or a pharmaceutically acceptable salt thereof.

14. The compound of any one of claims 1-4 and 10, having a structure according to Formula IId 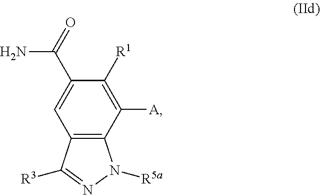
or a pharmaceutically acceptable salt thereof.

15. The compound of any one of claims 1-4, or a pharmaceutically acceptable salt thereof, wherein U is C, V is N, and W is C.

16. The compound of any one of claims 1-4 and 15, having a structure according to Formula IIIa 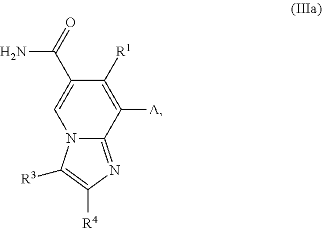
or a pharmaceutically acceptable salt thereof.

17. The compound of any one of claims 1-4 and 15, having a structure according to Formula IIIa 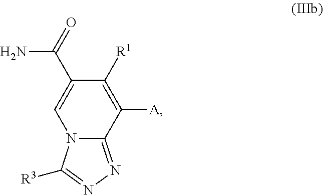
or a pharmaceutically acceptable salt thereof.

18. The compound of any one of claims 1-17, or a pharmaceutically acceptable salt thereof, wherein R³, R^{3a}, R⁴, R^{4a}, and R⁵ are independently selected from the group consisting of hydrogen, halogen, cyano, C₁₋₆ alkyl, C₁₋₆ alkoxy, C₃₋₈ cycloalkyl, C₁₋₆ haloalkyl, —COO(C₁₋₆ alkyl), —CON(R⁷)₂, C₆₋₁₄ aryl, 5- to 14-membered heteroaryl, and -L-(5- to 14-membered heterocyclyl).

19. The compound of any one of claims 1-18, or a pharmaceutically acceptable salt thereof, wherein ring A is C₆₋₁₄ aryl substituted with two or three R⁶, wherein one R⁶ is hydroxy and one or two R⁶ are C₁₋₆ alkyl.

20. The compound of claim 19, or a pharmaceutically acceptable salt thereof, wherein ring A is selected from the group consisting of 3-hydroxy-2,6-dimethylphenyl, 3-hydroxy-2-methylphenyl, 5-hydroxy-2-methylphenyl, and 3-hydroxy-2,4,6-trimethylphenyl.

21. The compound of any one of claims 1-18, or a pharmaceutically acceptable salt thereof, wherein ring A is 5- or 6-membered heteroaryl substituted with one or two R⁶, each of which is independently selected from the group consisting of halogen and C₁₋₆ alkyl.

22. The compound of claim 21, or a pharmaceutically acceptable salt thereof, wherein ring A is 5- or 6-membered heteroaryl substituted with two R⁶.

23. The compound of claim 21, or a pharmaceutically acceptable salt thereof, wherein ring A is selected from the group consisting of 5-chloro-4-methylpyridin-3-yl, 5-fluoro-4-methylpyridin-3-yl, 5-chloro-4-trifluoromethylpyridin-3-yl, 5-hydroxy-4-methylpyridin-3-yl, 5-fluoro-4-methoxypyridin-3-yl, and 4,5-difluoro-pyridin-3-yl.

24. The compound of any one of claims 1-18, or a pharmaceutically acceptable salt thereof, wherein ring A imidazopyridinyl.

25. A compound, or a pharmaceutically acceptable salt thereof, selected from any of the compounds in Table 1.

26. A pharmaceutical composition comprising a compound according to any one of claims 1-25, or a pharmaceutically acceptable salt thereof, and one or more pharmaceutically acceptable excipients.

27. A method for treating cancer comprising administering a therapeutically effective amount of a compound according to any one of claims 1-25, or a pharmaceutically acceptable salt thereof, to a subject in need thereof.

28. A process for making a compound of Formula I, 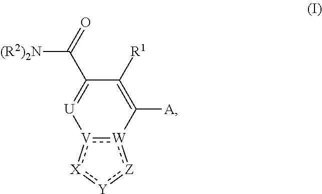
or a pharmaceutically acceptable salt thereof, comprising
(a) combining a compound of Formula (b) and a compound of Formula (c) to form a compound of Formula (d) 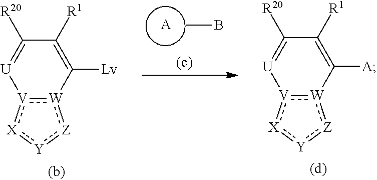
and
(b) converting the compound of Formula (d) to yield the compound of Formula I, 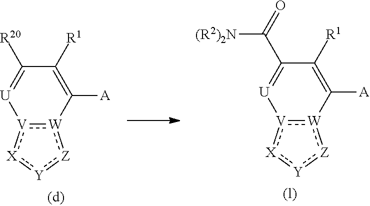
wherein
Lv is a leaving group;
the compound of Formula (c) is a boronic acid or boronic ester of ring A;
each [symbol] represents a single bond, a double bond, or a delocalized π bond;
U is selected from the group consisting of N and CH;
V and W are independently selected from the group consisting of N and C;
X is selected from the group consisting of CR³, N, NR^{3a}, S, and O;
Y is selected from the group consisting of CR⁴, N, NR^{4a}, S, and O;
Z is selected from the group consisting of CR⁵, N, NR^{5a}, S, and O;
ring A is
3- to 14-membered carbocyclyl substituted with 1, 2, 3, 4, or 5 R⁶, or
5- to 14-membered heterocyclyl comprising at least one oxygen atom, nitrogen atom, or sulfur atom, wherein the heterocyclyl is substituted with 0, 1, 2, 3, or 4 R⁶;
R¹ is selected from the group consisting of amino, hydroxy, and hydrogen;
each R² is independently selected from the group consisting of hydrogen and C₁₋₆ alkyl;
R³, R⁴, and R⁵ are independently selected from the group consisting of hydrogen, amino, hydroxy, halogen, cyano, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, —NO₂, —CO(C₁₋₆ alkyl), —COOH, —COO(C₁₋₆ alkyl), —CON(R⁷)₂, -L-(5- to 12-membered heterocyclyl), -L-(5- to 14-membered heteroaryl, and -L-(C₆₋₁₄ aryl), wherein the 5- to 12-membered heterocyclyl, 5- to 14-membered heteroaryl, and C₆₋₁₄ aryl are independently substituted with 0, 1, 2, 3, or 4 R⁶,
each L is independently selected from the group consisting of a covalent bond, —O—, C₁₋₆ alkylene, and NR^{a};
any two adjacent R³, R⁴, and R⁵ are optionally taken together with the carbon atoms to which they are attached to form a 5- or 6-membered carbocyle or heterocycle, each of which is optionally substituted with 0, 1, 2, 3, or 4 R⁶;
R^{3a}, R^{4a}, R^{5a}, and R⁷ are independently selected from the group consisting of hydrogen, C₁₋₆ alkyl, C₁₋₆ haloalkyl, C₁₋₆ hydroxyalkyl, C₁₋₆ alkoxy, C₁₋₆ haloalkoxy, C₃₋₈ cycloalkyl, -L-(5- to 12-membered heterocyclyl), -L-(5- to 14-membered heteroaryl, and -L-(C₆₋₁₄ aryl), wherein the C₃₋₈ cycloalkyl, 5- to 12-membered heterocyclyl, 5- to 14-membered heteroaryl, and C₆₋₁₄ aryl are independently substituted with 0, 1, 2, 3, or 4 R⁶;
each R⁶ is independently selected from the group consisting of hydroxy, amino, cyano, halogen, C₁₋₆ alkyl, C₁₋₆ haloalkyl, C₁₋₆ hydroxyalkyl, C₁₋₆ alkoxy, oxo, —NO₂, —CHO, —CO(C₁₋₆ alkyl), —COOR^{a}, —CON(R^{a})₂, —SO₂(C₁₋₆ alkyl), and —SO₂N(R^{a})₂;
R²⁰ is —COOR^{a}, —COONH₂, or CN; and
each R^{a} is independently selected from the group consisting of hydrogen, C₁₋₆ alkyl, and C₁₋₆ haloalkyl.

29. The process of claim 28, wherein the process further comprises converting a compound of Formula (a) to the compound of Formula (b)

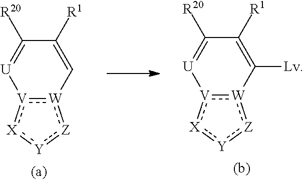

30. The process of claim 28 or claim 29, wherein the compound of Formula (c)

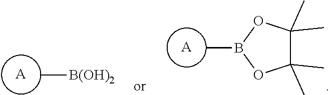

31. The process of any one of claims 28-30, wherein Lv is halogen.
